(19) United States
(12) Patent Application Publication
(10) Pub. No.: US 2013/0158075 A1
(43) Pub. Date: Jun. 20, 2013

(54) HETEROCYCLIC COMPOUNDS AS DGAT1 INHIBITORS

(71) Applicants: Rajiv Sharma, Mumbai (IN); Kishorkumar Shivajirao Kadam, Mumbai (IN); Ravindra Dnyandev Jadhav, Mumbai (IN); Shivaji Sadashiv Kandre, Mumbai (IN); Amol Gupte, Mumbai (IN)

(72) Inventors: Rajiv Sharma, Mumbai (IN); Kishorkumar Shivajirao Kadam, Mumbai (IN); Ravindra Dnyandev Jadhav, Mumbai (IN); Shivaji Sadashiv Kandre, Mumbai (IN); Amol Gupte, Mumbai (IN)

(73) Assignee: PIRAMAL ENTERPRISES LIMITED, Mumbai (IN)

(21) Appl. No.: 13/820,240

(22) PCT Filed: Aug. 31, 2011
(86) PCT No.: PCT/IB11/53810

§ 371 Date: Mar. 1, 2013

Related U.S. Application Data
(60) Provisional application No. 61/379,760, filed on Sep. 3, 2010.

(51) Int. Cl. C07D 417/04; C07D 285/12; C07D 263/32; C07D 231/12; C07D 271/107; C07D 277/587

(52) U.S. Cl. CPC C07D 417/04; C07D 271/107; C07D 277/587; C07D 263/32; C07D 231/12; C07D 285/12
USPC 514/326; 514/363; 514/364; 514/365; 514/374; 514/406; 546/209; 548/131; 548/136; 548/143; 548/204; 548/235; 548/377.1

(57) ABSTRACT

The present invention relates to heterocyclic compounds of formula 1, in all their stereoisomeric and tautomeric forms; and their pharmaceutically acceptable salts, solvates, polymorphs, prodrugs, carboxylic acid isosteres and N-oxides. The invention also relates to processes for the manufacture of the heterocyclic compounds and to pharmaceutical compositions containing them. The said compounds and their pharmaceutical compositions are useful in the prevention and treatment of diseases or disorders mediated by diacylglycerol acyltransferase (DGAT), particularly DGAT1. The present invention further provides a method of treatment of such diseases or disorders by administering a therapeutically effective amount of said compounds or their pharmaceutical compositions, to a mammal in need thereof.

### FIELD OF THE INVENTION

The present invention relates to heterocyclic compounds, to processes for their preparation, pharmaceutical compositions containing them, and their use in the prevention and treatment of diseases or disorders mediated by diacylglycerol acyltransferase (DGAT), particularly DGAT1.

### BACKGROUND OF THE INVENTION

Obesity is a disease of energy imbalance, when energy input is more than output. Excess energy is stored in the form of triglycerides (TGs) in the adipose tissue. Increased adipose cell size causes hypertrophic obesity and increased cell number causes hyperplastic obesity characteristic of a more severe condition. The key causes of obesity are the increased consumption of energy-rich but nutrient-poor diets (like saturated fats and sugars) and reduced physical activity. 65% of the US population is overweight, where body mass index (BMI) is greater than 25 and approximately 25% of them are obese, having BMI >30. The prevalence of obesity has increased dramatically over the last decade. Obesity leads to increased risk of chronic diseases such as type 2 diabetes, insulin resistance, hypertension, stroke, cardiovascular diseases, respiratory problems, gall bladder disease, osteoarthritis, sleep apnea and certain cancers (Expert Opin. Ther. Targets, 2009, 13, 2, 195-207). The increasing evidence that severe obesity has a genetic basis, resulting in maintaining and defending an elevated weight, may explain why long-term weight loss is very difficult to achieve. This has strengthened the argument that severe obesity should be treated with pharmacological agents along with conventional diet and exercise regimes.

Diacylglycerol acyltransferase (DGAT) is an enzyme that catalyses the biosynthesis of triglyceride at the final step of the process, converting diacylglycerol (DAG) and fatty acyl-coenzyme A (CoA) into triglyceride. The enzymatic activity is present in all cell types because of the necessity of producing triglyceride for cellular needs. The amount of triglyceride synthesized varies from cell to cell, with the adipocytes, hepatocytes and intestinal enterocytes producing much more triglyceride, for storage or incorporation into lipoproteins, than other cell types. Because of its critical role in the biosynthesis of triglyceride, a neutral lipid that is the densest form of energy storage in animals, alteration of the expression and/or activity of DGAT in any of the tissues or organs would be expected to perturb the systemic energy metabolism. Diacyl glycerolacyltransferase 1 (DGAT1) is one of two known DGAT enzymes that catalyze the final step in triglyceride synthesis. Although most tissues generate triacylglycerols, DGAT1 is known to be highly expressed in the intestine and adipose with lower levels in the liver and muscle. Inhibition of DGAT1 in each of these tissues (intestine, adipose, liver and muscle) would inhibit triacylglycerol synthesis and may reverse the pathophysiology of excessive lipid accumulation in human metabolic disease.

Inhibitors of varying structural types of DGAT1 have been reported to be potential agents for the treatment for obesity and other disorders. The particular interest in DGAT1 inhibition stems from the reported phenotype of DGAT1 deficient (Dgat1−/−) mice. These animals are viable, resistant to weight gain when fed a high-fat diet, and show increased insulin and leptin sensitivity (Nature Genetics, 2000, 25, 87-90). Resistance to weight gain results from increased energy expenditure rather than decreased food intake (the animals are in fact hyperphagic) and is associated with loss of adipose rather than lean tissue mass. Most aspects of this phenotype can be reproduced in rodents by treatment with a potent and selective small molecule inhibitor of DGAT1. DGAT1 inhibitors may also have utility for the treatment of skin disorders such as acne (The Journal of Biological Chemistry, 2009, 284, 7, 4292-4299).

XP620 (BMS) has been reported to be a selective DGAT1 inhibitor, which is able to block DGAT1 mediated retinyl-ester formation in Caco-2 cells. The potency against DGAT1 was in the order of 100 nM with no activity against DGAT2.

Other small-molecule inhibitors reported are aryl alkyl acids from Bayer, phosphonic acid diesters from Otsuka, substituted ureas from Sankyo, pyrrolo[1,2-b]pyridazine derivatives from Tularik (now Amgen) and oxadiazoles from AstraZeneca (Expert Opin. Ther. Targets, 2006, 10, 5, 749-757).

The PCT publication, WO2007016538 discloses biphenyl amino acid derivatives, and pharmaceutical salts and esters thereof, that have utility in the inhibition of DGAT1 and in the treatment of obesity and related diseases.

The Japanese patent publication, JP2008255024 discloses biarylamine derivatives for the inhibition of DGAT1.

U.S. Pat. No. 7,625,914 discloses substituted propanoic acid derivatives as modulators of PPAR-γ type receptors, useful for treating conditions or disorders such as cardiovascular diseases, immune diseases and/or diseases associated with lipid metabolism.

Despite the recent advances in this field, there still exists a need for effective and safe pharmacotherapy for obesity.

### SUMMARY OF THE INVENTION

The present invention relates to heterocyclic compounds, processes for their preparation and their use in the prevention and treatment of diseases or disorders mediated by diacylglycerol acyltransferase (DGAT), particularly DGAT1.

According to one aspect of the present invention, there are provided heterocyclic compounds of formula 1 (as described herein below), as well as stereoisomers, tautomeric forms, pharmaceutically acceptable salts, solvates, polymorphs, prodrugs, carboxylic acid isosteres and N-oxides thereof.

According to another aspect of the present invention, there are provided processes for producing the heterocyclic compounds of formula 1.

According to a further aspect, there is provided the use of heterocyclic compounds of formula 1 in the prevention or treatment of diseases or disorders mediated by diacylglycerol acyltransferase (DGAT), particularly DGAT1.

According to another aspect of the present invention, there are provided pharmaceutical compositions including heterocyclic compounds of formula 1 as active ingredient.

According to yet another aspect of the present invention, there is provided a method for the prevention or treatment of diseases or disorders mediated by diacylglycerol acyltransferase (DGAT), particularly DGAT1, the method including administering to a mammal in need thereof a therapeutically effective amount of a compound of formula 1.

According to a further aspect of the present invention, there is provided use of compounds of formula 1 for the manufacture of medicaments, which are useful for the prevention or treatment of diseases or disorders mediated by diacylglycerol acyltransferase (DGAT), particularly DGAT1.

### DETAILED DESCRIPTION OF THE INVENTION

The present invention provides compounds of formula 1:

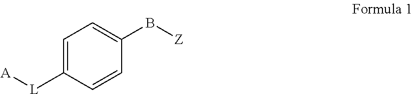

in all their stereoisomeric and tautomeric forms; and their pharmaceutically acceptable salts, solvates, polymorphs, prodrugs, carboxylic acid isosteres and N-oxides;
wherein,
Z is selected from:

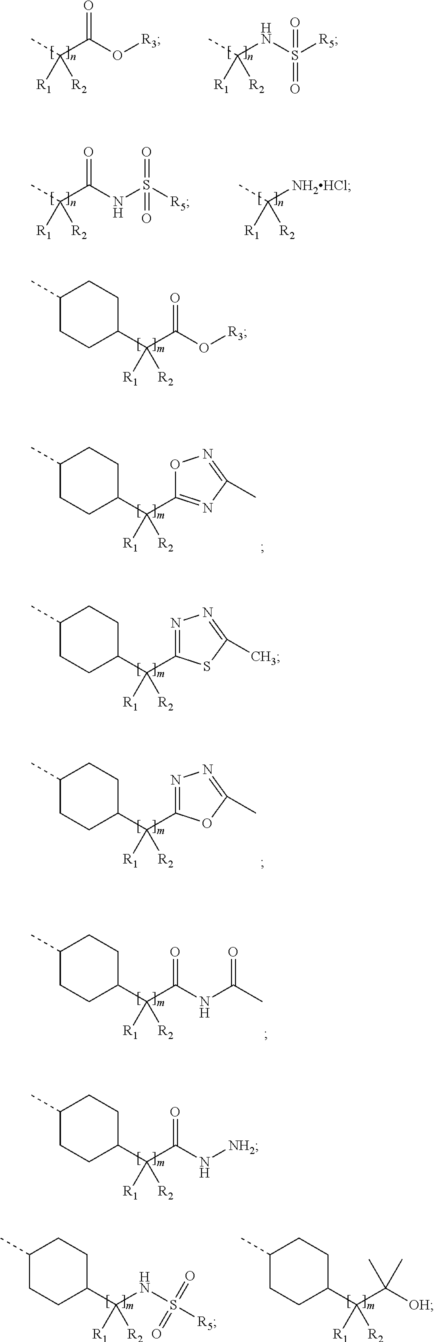

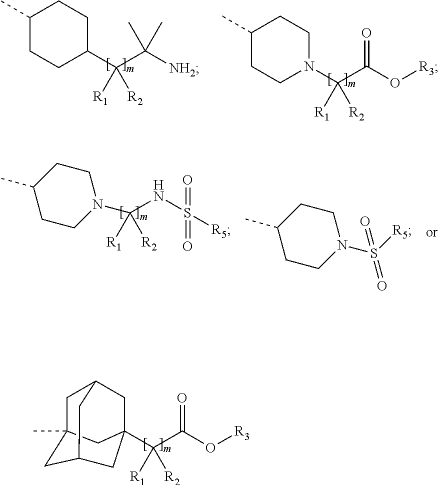

[symbol]

indicates the point of attachment;
n is an integer selected from 1-5;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring;
R₃ is hydrogen or (C₁-C₁₂)-alkyl;
R₅ is selected from hydrogen, (C₁-C₁₂)-alkyl, CF₃, (C₃-C₇)-cycloalkyl, aryl or heterocyclyl;
B is a 5-membered heteroaryl ring represented by any one of the general structures (i) to (x);

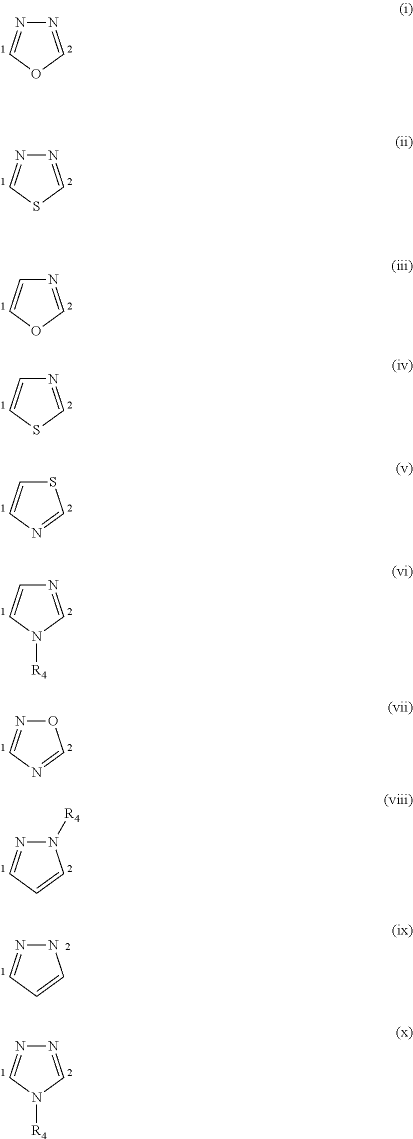

wherein 1 and 2 are the points of attachment of B to phenyl and to Z respectively and R₄ is selected from hydrogen, (C₁-C₁₂)-alkyl or aryl; or B is a 6-membered heteroaryl ring containing 1 or 2 N-atoms, wherein the 6-membered heteroaryl ring may be unsubstituted or substituted with one or more groups selected from halogen, hydroxy, (C₁-C₁₂)-alkoxy, cyano, nitro, (C₁-C₁₂)-alkyl, (C₂-C₁₂)-alkenyl, (C₂-C₁₂)-alkynyl, (C₃-C₁₂)-cycloalkyl, aryl, aryloxy, heterocyclyl or O-heterocyclyl;
L is selected from *NHC(O)NH, *N(CH₃)C(O)NH *NHC(S)NH, *SO₂NH, *CONH or *NH(C═NR₆)NH, wherein * indicates the point of attachment of L to A, and R₆ is selected from hydrogen, methyl, cyano or nitro;
A is selected from (C₁-C₁₂)-alkyl, (C₃-C₁₂)-cycloalkyl, aryl or heterocyclyl;
wherein,
(C₁-C₁₂)-alkyl is unsubstituted or substituted with one or more groups selected from halogen, hydroxy, (C₁-C₁₂)-alkoxy, cyano, nitro, (C₃-C₁₂)-cycloalkyl, aryl, heterocyclyl, C(O)Rₚ, C(O)ORₚ, NRₚR_{q}, C(O)NRₚR_{q}, SRₚ, S(O)Rₚ or SO₂Rₚ;
(C₃-C₁₂)-cycloalkyl is unsubstituted or substituted with one or more groups selected from halogen, hydroxy, (C₁-C₁₂)-alkoxy, cyano, nitro, aryl, heterocyclyl, C(O)Rₚ, C(O)ORₚ, NRₚR_{q}, C(O)NRₚR_{q}, SRₚ, S(O)Rₚ or SO₂Rₚ;
aryl is unsubstituted or substituted with one or more groups selected from halogen, hydroxy, (C₁-C₁₂)-alkoxy, cyano, nitro, (C₁-C₁₂)-alkyl, OCF₃, CF₃, (C₂-C₁₂)-alkenyl, (C₂-C₁₂)-alkynyl, (C₃-C₁₂)-cycloalkyl, aryl, aryloxy, heterocyclyl, O-heterocyclyl, C(O)Rₚ, C(O)ORₚ, NRₚR_{q}, C(O)NRₚR_{q}, SRₚ, S(O)Rₚ or SO₂Rₚ; or aryl may be fused with an unsubstituted or substituted 5 or 6-membered cycloalkyl ring optionally containing one or more heteroatoms selected from O, N or S;
heterocyclyl is unsubstituted or substituted with one or more groups selected from halogen, hydroxy, (C₁-C₁₂)-alkoxy, cyano, nitro, (C₁-C₁₂)-alkyl, (C₂-C₁₂)-alkenyl, (C₂-C₁₂)-alkynyl, (C₃-C₁₂)-cycloalkyl, aryl, aryloxy, heterocyclyl, O-heterocyclyl, C(O)Rₚ, C(O)ORₚ, NRₚR_{q}, C(O)NRₚRₚ, SRₚ, S(O)Rₚ or SO₂Rₚ;
Rₚ and R_{q} are independently selected from hydrogen, (C₁-C₁₂)-alkyl, aryl, aralkyl or heterocyclyl, or Rₚ and R_{q} together with the N to which they are attached optionally form a 3 to 7 membered ring;
with a proviso that A is not a methyl group.

### DEFINITIONS

As used herein, the term “alkyl” whether used alone or as part of a substituent group, refers to the radical of saturated aliphatic groups, including straight or branched-chain alkyl groups. An alkyl group can have a straight chain or branched chain containing 1 to 12 carbon atoms. Alkyl groups include methyl, ethyl, n-propyl, isopropyl, n-butyl, t-butyl, iso-butyl, sec-butyl, neo-pentyl, n-pentyl, n-heptyl, n-octyl, n-nonyl and n-decyl groups.

A substituted alkyl refers to an alkyl group substituted with one or more groups selected from halogen, hydroxy, cyano, nitro, unsubstituted or substituted (C₁-C₁₂)-alkoxy, unsubstituted or substituted cycloalkyl, unsubstituted or substituted aryl, unsubstituted or substituted heterocyclyl, C(O)Rₚ, C(O)ORₚ, SRₚ, S(O)Rₚ, SO₂Rₚ, NRₚR_{q} or C(O)NRₚRₚ; wherein Rₚ and R_{q} are independently selected from hydrogen, unsubstituted or substituted (C₁-C₁₂) alkyl, unsubstituted or substituted aryl, unsubstituted or substituted aralkyl and unsubstituted or substituted heterocyclyl, or Rₚ and R_{q} together with the N to which they are attached optionally form a 3 to 7 membered ring. Examples of substituted alkyls include benzyl, hydroxymethyl, hydroxyethyl, 2-hydroxyethyl, N-morpholinomethyl, N-indolomethyl, piperidinylmethyl, trifluoromethyl and aminoethyl.

As used herein, the term “alkenyl” whether used alone or as part of a substituent group, refers to a straight or branched chain hydrocarbon radical containing the indicated number of carbon atoms and at least one carbon-carbon double bond (two adjacent sp² carbon atoms). For example, (C₂-C₁₂)-alkenyl refers to an alkenyl group having 2 to 12 carbon atoms. Similarly, (C₂-C₆)-alkenyl refers to an alkenyl group having 2 to 6 carbon atoms. Depending on the placement of double bond and substituents if any, the geometry of the double bond may be entgegen (E), or zusammen (Z), cis or trans. Examples of alkenyl include, but are not limited to, vinyl, allyl and 2-propenyl.

A substituted alkenyl refers to an alkenyl group substituted with one or more groups selected from halogen, hydroxy, cyano, nitro, unsubstituted or substituted (C₁-C₁₂)-alkoxy, unsubstituted or substituted aryl, unsubstituted or substituted heterocyclyl, C(O)Rₚ, C(O)ORₚ, SRₚ, S(O)Rₚ, SO₂Rₚ, NRₚR_{q} or C(O)NRₚR_{q}; wherein Rₚ and R_{q} are independently selected from hydrogen, unsubstituted or substituted (C₁-C₁₂) alkyl, unsubstituted or substituted aryl, unsubstituted or substituted aralkyl and unsubstituted or substituted heterocyclyl, or Rₚ and R_{q} together with the N to which they are attached optionally form a 3 to 7 membered ring.

As used herein, the term “alkynyl” whether used alone or as part of a substituent group, refers to a straight or branched chain hydrocarbon radical containing the indicated number of carbon atoms and at least one carbon-carbon triple bond (two adjacent sp carbon atoms). For example, (C₂-C₁₂)-alkynyl refers to an alkynyl group having 2-12 carbon atoms. Examples of alkynyl include, but are not limited to, ethynyl, 1-propynyl, 3-propynyl and 3-butynyl.

A substituted alkynyl refers to an alkynyl group substituted with one or more groups selected from halogen, hydroxy, cyano, nitro, unsubstituted or substituted (C₁-C₁₂)-alkoxy, unsubstituted or substituted aryl, unsubstituted or substituted heterocyclyl, C(O)Rₚ, C(O)ORₚ, SRₚ, S(O)Rₚ, SO₂Rₚ, NRₚR_{q} or C(O)NRₚR_{q}; wherein Rₚ and R_{q} are independently selected from hydrogen, unsubstituted or substituted (C₁-C₁₂) alkyl, unsubstituted or substituted aryl, unsubstituted or substituted aralkyl and unsubstituted or substituted heterocyclyl, or Rₚ and R_{q} together with the N to which they are attached optionally form a 3 to 7 membered ring.

As used herein, the term “alkoxyl” or “alkoxy” refers to a (C₁-C₁₂)-alkyl having an oxygen radical attached thereto. Representative alkoxy groups include methoxy, ethoxy, propoxy, isopropoxy, isobutoxy and tert-butoxy.

A substituted alkoxy refers to an alkoxy group in which the alkyl is substituted with one or more groups selected from halogen, hydroxy, cyano, nitro, unsubstituted or substituted aryl, unsubstituted or substituted heterocyclyl, C(O)Rₚ, C(O)ORₚ, SRₚ, S(O)Rₚ, SO₂Rₚ, NRₚR_{q} and C(O)NRₚR_{q}; wherein Rₚ and R_{q} are independently selected from hydrogen, unsubstituted or substituted (C₁-C₁₂) alkyl, unsubstituted or substituted aryl, unsubstituted or substituted aralkyl and unsubstituted or substituted heterocyclyl, or Rₚ and R_{q} together with the N to which they are attached optionally form a 3 to 7 membered ring. Examples of substituted alkoxy are trifluoromethoxy, 2-cyanoethoxy and benzyloxy group. A benzyloxy group refers to a benzyl having an oxygen radical attached thereto.

The term “(C₃-C₁₂) cycloalkyl” refers to monocyclic, bicyclic or tricyclic hydrocarbon groups of 3-12 carbon atoms, which may be optionally bridged such as adamantyl.

The term “(C₃-C₇) cycloalkyl” refers to monocyclic hydrocarbon groups of 3-7 carbon atoms.

A substituted (C₃-C₁₂) cycloalkyl refers to a “(C₃-C₁₂) cycloalkyl” substituted by one or more substituents such as halogen, hydroxy, unsubstituted or substituted (C₁-C₁₂)-alkyl, (C₁-C₁₂)-alkoxy cyano, nitro, unsubstituted or substituted aryl, unsubstituted or substituted heterocyclyl, C(O)Rₚ, C(O)ORₚ, SRₚ, S(O)Rₚ, SO₂Rₚ, NRₚR_{q} or C(O)NRₚR_{q}; wherein Rₚ and R_{q} are independently selected from hydrogen, unsubstituted or substituted (C₁-C₁₂) alkyl, unsubstituted or substituted aryl, unsubstituted or substituted aralkyl and unsubstituted or substituted heterocyclyl, or Rₚ and R_{q} together with the N to which they are attached optionally form a 3 to 7 membered ring.

The term “aryl” as used herein refers to monocyclic or polycyclic hydrocarbon groups having 6 to 14 ring carbon atoms in which the carbocyclic ring(s) present have a conjugated pi electron system. Examples of (C₆-C₁₀-aryl residues are phenyl, naphthyl, fluorenyl or anthracenyl. Examples of (C₆-C₁₀)-aryl residues are phenyl or naphthyl. Aryl groups can be unsubstituted or substituted by one or more, for example 1, 2, 3, 4 or 5, identical or different substituents selected from halogen, hydroxy, cyano, nitro, unsubstituted or substituted (C₁-C₁₂) alkyl, unsubstituted or substituted (C₂-C₁₂)-alkenyl, unsubstituted or substituted (C₂-C₁₂)-alkynyl, unsubstituted or substituted (C₁-C₁₂)-alkoxy, unsubstituted or substituted cycloalkyl, unsubstituted or substituted aryl, unsubstituted or substituted aryloxy, unsubstituted or substituted heterocyclyl, O-heterocyclyl, OCF₃, CF₃, C(O)Rₚ, C(O)ORₚ, SRₚ, S(O)Rₚ, SO₂Rₚ, NRₚR_{q} or C(O)NRₚR_{q}; wherein Rₚ and R_{q} are independently selected from hydrogen, unsubstituted or substituted (C₁-C₁₂) alkyl, unsubstituted or substituted aryl, unsubstituted or substituted aralkyl and unsubstituted or substituted heterocyclyl, or Rₚ and R_{q} together with the N to which they are attached optionally form a 3 to 7 membered ring. In monosubstituted phenyl residues the substituent can be located in the 2-position, the 3-position or the 4-position. If the phenyl carries two substituents, they can be located in 2,3-position, 2,4-position, 2,5-position, 2,6-position, 3,4-position or 3,5-position. Examples of monosubstituted phenyl groups are biphenyl, 4-methylphenyl, 2-trifluoromethylphenyl, 4-trifluoromethoxyphenyl, 4-cyanophenyl and 3-nitrophenyl. Examples of disubstituted phenyl groups are 3,5-difluorophenyl and 3,4-dimethoxyphenyl.

As used herein, the term “aryloxy” refers to an aryl group having an oxygen radical attached thereto. The aryl of aryloxy group as used herein may also be defined as given herein above. Representative aryloxy groups include phenyloxy, 4-chlorophenoxy, 3,4-dimethoxy phenoxy, etc.

The term “aralkyl” refers to an aryl group bonded directly through an alkyl group, such as benzyl. The aryl of the aralkyl group may be unsubstituted or substituted as explained in the definition of substituted aryl herein above.

The term “heteroatom” as used herein includes nitrogen, oxygen and sulfur. Any heteroatom with unsatisfied valency is assumed to have a hydrogen atom to satisfy the valency. Heterocyclyl includes saturated heterocyclic ring systems, which do not contain any double bonds within the rings, as well as unsaturated heterocyclic ring systems, which contain one or more, for example, 3 double bonds within a ring, provided that the resulting mono, bi or tricyclic ring system is stable. The heterocyclyl group may, for example, have 1 or 2 oxygen atoms and/or 1 or 2 sulfur atoms and/or 1 to 3 nitrogen atoms in the ring. Examples of heterocyclyls include pyrrolyl, pyrrolidinyl, pyrazolyl, imidazolyl, pyrazinyl, piperazinyl, oxazolyl, isoxazolyl, thiazolyl, furyl, thienyl, pyridyl, pyrimidyl, piperidyl, benzothiazolyl, purinyl, benzimidazolyl, benzooxazolyl, indolyl, isoindolyl, isoquinolyl, morpholinyl, quinoxalinyl, and quinolyl. Aromatic heterocyclyl groups may also be referred to by the customary term “heteroaryl” for which all the definitions and explanations relating to heterocyclyl apply. Examples of a 6-membered heteroaryl group containing 1 or 2 N atoms are pyridine, pyrimidine, pyridazine and pyrazine.

A substituted heterocyclyl refers to a heterocyclyl substituted with one or more groups selected from halogen, hydroxy, cyano, nitro, unsubstituted or substituted (C₁-C₁₂)-alkyl, (C₂-C₁₂)-alkenyl, (C₂-C₁₂)-alkynyl, unsubstituted or substituted (C₁-C₁₂)-alkoxy, unsubstituted or substituted cycloalkyl, unsubstituted or substituted aryl, unsubstituted or substituted aryloxy, heterocyclyl, —O-heterocyclyl, C(O)Rₚ, C(O)ORₚ, SRₚ, S(O)Rₚ, SO₂Rₚ, NRₚR_{q} and C(O)NRₚRₚ; wherein Rₚ and R_{q} are independently selected from hydrogen, unsubstituted or substituted (C₁-C₁₂) alkyl, unsubstituted or substituted aryl, unsubstituted or substituted aralkyl and unsubstituted or substituted heterocyclyl or Rₚ and R_{q} together with the N to which they are attached optionally form a 3 to 7 membered ring.

The substituents may be present on either the ring carbon or the ring nitrogen atoms. The substituents can be present at one or more positions provided that a stable molecule results.

The term “halogen” refers to a fluorine, chlorine, bromine, or iodine atom.

The term “solvate” describes a complex wherein the compound is coordinated with a proportional amount of a solvent molecule. Specific solvates, wherein the solvent is water, are referred to as hydrates.

The term “tautomer” refers to the coexistence of two (or more) compounds that differ from each other only in the position of one (or more) mobile atoms and in electron distribution, for example, keto-enol tautomers.

Carboxylic acid isosteres refer to groups or molecules that have physical and chemical similarities to a carboxylic acid group, producing similar biological effects as those produced by a carboxylic acid group. Examples of carboxylic acid isosteres include groups selected from hydroxamic, acylcyanamide, phosphonate, sulfonate, sulfonamide, tetrazole, hydroxylisoxazole and oxadiazolone (The Practice of Medicinal Chemistry, Edited by Camille G. Wermuth, Second Edition, 2003, 189-214).

The term “N-oxide” as used herein refers to the oxide of the nitrogen atom of a nitrogen-containing heteroaryl or heterocycle. N-oxide can be formed in presence of an oxidizing agent for example peroxide such as m-chloro-perbenzoic acid or hydrogen peroxide. N-oxide is also known as amine-N-oxide, and is a chemical compound that contains N→O bond.

It will be understood that “substitution” or “substituted with” includes the implicit proviso that such substitution is in accordance with permitted valence of the substituted atom and the substituent, as well as represents a stable compound, which does not readily undergo undesired transformation such as by rearrangement, cyclization, or elimination.

As used herein, the term “compound of formula 1” includes all the stereoisomeric and tautomeric forms and mixtures thereof in all ratios, and their pharmaceutically acceptable salts, solvates, polymorphs, prodrugs, carboxylic acid isosteres and N-oxides.

### ASPECTS OF THE INVENTION

In an aspect, the present invention provides compounds of formula 1 represented by compounds of formula 1a,

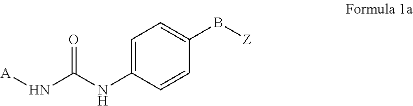

in all their stereoisomeric and tautomeric forms; and their pharmaceutically acceptable salts, solvates, polymorphs, prodrugs, carboxylic acid isosteres and N-oxides;
wherein;
Z is selected from:

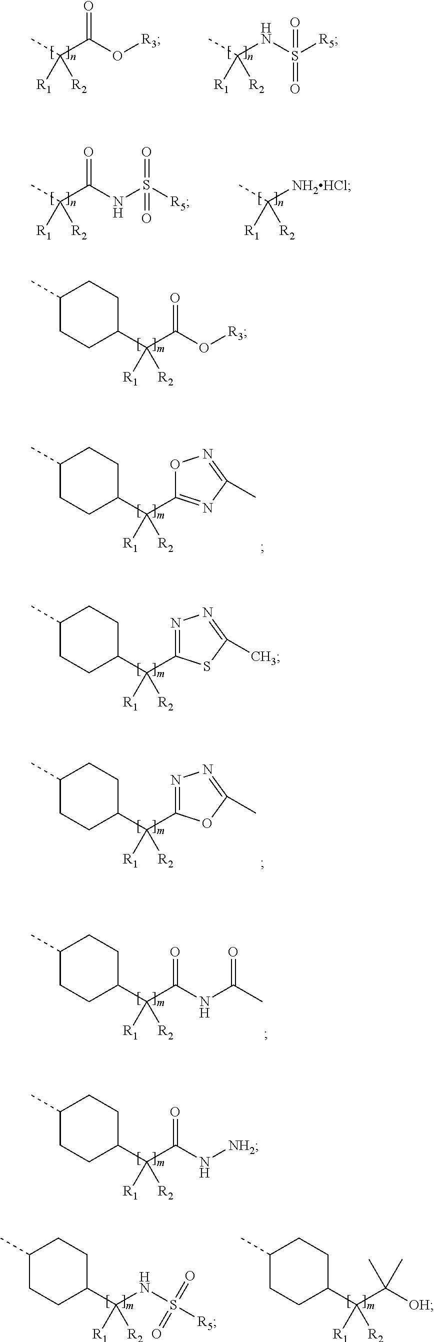

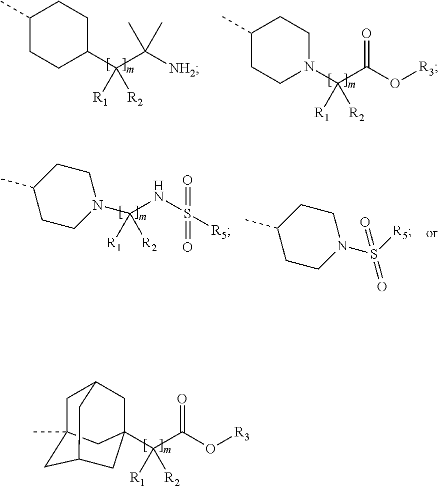

[symbol]

indicates the point of attachment;
n is an integer selected from 1-5;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring;
R₃ is hydrogen or (C₁-C₁₂)-alkyl;
R₅ is selected from hydrogen, (C₁-C₁₂)-alkyl, CF₃, (C₃-C₇)-cycloalkyl, aryl or heterocyclyl;
B is a 5-membered heteroaryl ring represented by any one of the general structures (i) to (x);

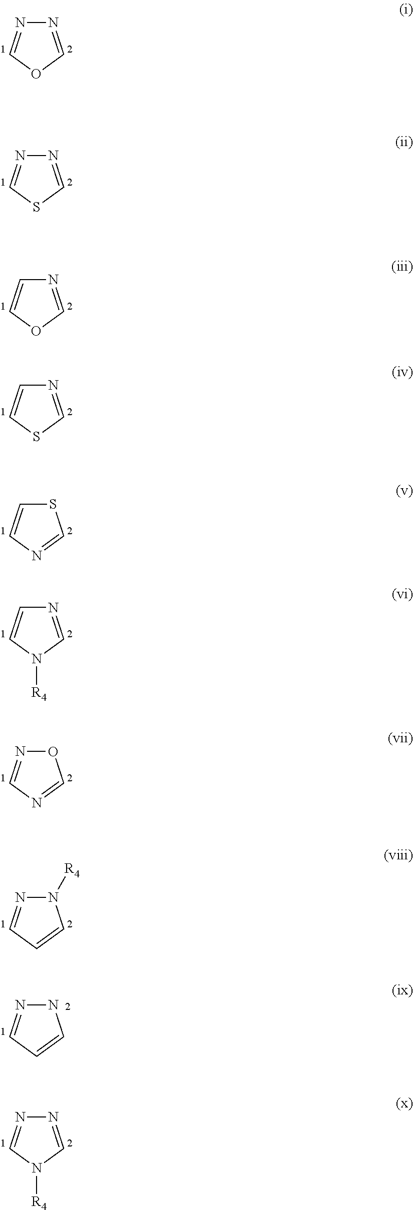

wherein 1 and 2 are the points of attachment of B to phenyl and to Z respectively and R₄ is selected from hydrogen, (C₁-C₁₂)-alkyl or aryl; or B is a 6-membered heteroaryl ring containing 1 or 2 N-atoms, wherein the 6-membered heteroaryl ring may be unsubstituted or substituted with one or more groups selected from halogen, hydroxy, (C₁-C₁₂)-alkoxy, cyano, nitro, (C₁-C₁₂)-alkyl, (C₂-C₁₂)-alkenyl, (C₂-C₁₂)-alkynyl, (C₃-C₁₂)-cycloalkyl, aryl, aryloxy, heterocyclyl or O-heterocyclyl; and
A is selected from (C₁-C₁₂)-alkyl, (C₃-C₁₂)-cycloalkyl, aryl or heterocyclyl;
wherein,
(C₁-C₁₂-alkyl is unsubstituted or substituted with one or more groups selected from halogen, hydroxy, (C₁-C₁₂)-alkoxy, cyano, nitro, (C₃-C₁₂)-cycloalkyl, aryl, heterocyclyl, C(O)Rₚ, C(O)ORₚ, NRₚR_{q}, C(O)NRₚR_{q}, SRₚ, S(O)Rₚ or SO₂Rₚ;
(C₃-C₁₂)-cycloalkyl is unsubstituted or substituted with one or more groups selected from halogen, hydroxy, (C₁-C₁₂)-alkoxy cyano, nitro, aryl, heterocyclyl, C(O)Rₚ, C(O)ORₚ, NRₚR_{q}, C(O)NRₚR_{q}, SRₚ, S(O)Rₚ or SO₂Rₚ;
aryl is unsubstituted or substituted with one or more groups selected from halogen, hydroxy, (C₁-C₁₂)-alkoxy, cyano, nitro, (C₁-C₁₂)-alkyl, OCF₃, CF₃, (C₂-C₁₂)-alkenyl, (C₂-C₁₂)-alkynyl, (C₃-C₁₂)-cycloalkyl, aryl, aryloxy, heterocyclyl, O-heterocyclyl, C(O)Rₚ, C(O)ORₚ, NRₚR_{q}, C(O)NRₚR_{q}, SRₚ, S(O)Rₚ or SO₂Rₚ; or aryl may be fused with an unsubstituted or substituted 5 or 6-membered cycloalkyl ring optionally containing one or more heteroatoms selected from O, N or S;
heterocyclyl is unsubstituted or substituted with one or more groups selected from halogen, hydroxy, (C₁-C₁₂)-alkoxy, cyano, nitro, (C₁-C₁₂)-alkyl, (C₂-C₁₂)-alkenyl, (C₂-C₁₂)-alkynyl, (C₃-C₁₂)-cycloalkyl, aryl, aryloxy, heterocyclyl, O-heterocyclyl, C(O)Rₚ, C(O)ORₚ, NRₚR_{q}, C(O)NRₚR_{q}, SRₚ, S(O)Rₚ or SO₂Rₚ;
Rₚ and R_{q} are independently selected from hydrogen, (C₁-C₁₂)-alkyl, aryl, aralkyl or heterocyclyl, or Rₚ and R_{q} together with the N to which they are attached optionally form a 3 to 7 membered ring;
with the proviso that A is not a methyl group.

In a second aspect, the present invention provides compounds of formula 1 represented by compounds of formula 1a, wherein,

### B is

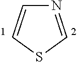

wherein 1 and 2 are the points of attachment of B to phenyl and to Z respectively;

### Z is

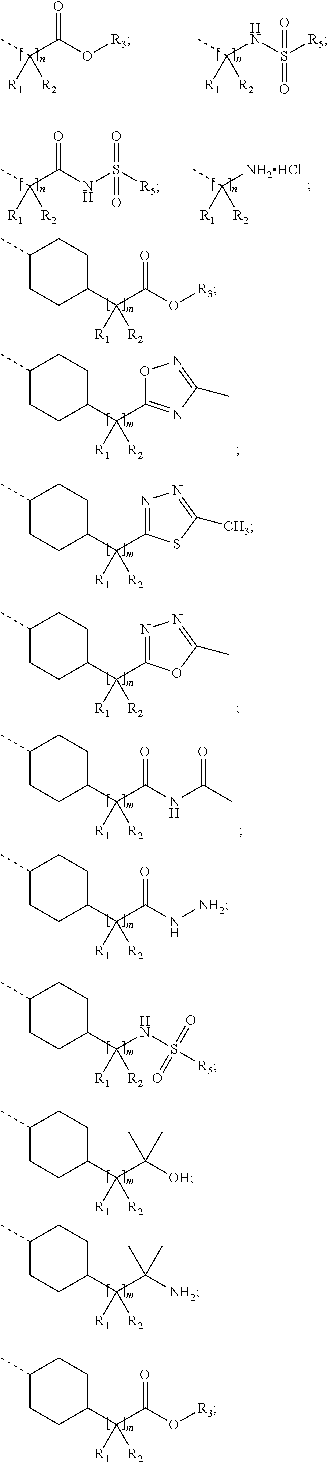

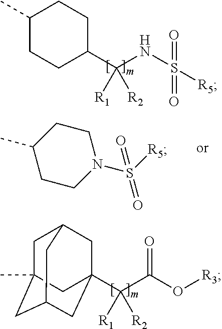

[symbol]

indicates the point of attachment;
n is an integer selected from 1-5;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring;
R₃ is hydrogen or (C₁-C₁₂)-alkyl;
R₅ is selected from hydrogen, (C₁-C₁₂)-alkyl, CF₃, (C₃-C₇)-cycloalkyl, aryl or heterocyclyl; and
A is selected from (C₁-C₁₂)-alkyl, (C₃-C₁₂)-cycloalkyl, aryl or heterocyclyl;
wherein,
(C₁-C₁₂)-alkyl is unsubstituted or substituted with one or more groups selected from halogen, hydroxy, (C₁-C₁₂)-alkoxy, cyano, (C₃-C₁₂)-cycloalkyl, aryl or heterocyclyl;
(C₃-C₁₂)-cycloalkyl is unsubstituted or substituted with one or more groups selected from halogen, hydroxy, (C₁-C₁₂)-alkoxy, cyano, aryl or heterocyclyl;
aryl is unsubstituted or substituted with one or more groups selected from halogen, hydroxy, (C₁-C₁₂)-alkoxy, cyano, (C₁-C₁₂)-alkyl, OCF₃, CF₃, (C₃-C₁₂)-cycloalkyl, aryl, aryloxy, heterocyclyl or O-heterocyclyl; or aryl may be fused with an unsubstituted or substituted 5 or 6-membered cycloalkyl ring optionally containing one or more heteroatoms selected from O, N or S;
heterocyclyl is unsubstituted or substituted with one or more groups selected from halogen, hydroxy, (C₁-C₁₂)-alkoxy, cyano, (C₁-C₁₂)-alkyl, (C₃-C₁₂)-cycloalkyl, aryl, aryloxy, heterocyclyl or O-heterocyclyl;
with the proviso that A is not a methyl group.

In an embodiment of the second aspect, the present invention provides compounds of formula 1 represented by compounds of formula 1a, wherein

B and A are as defined in the second aspect of the invention;

### Z is

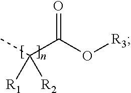

[symbol]

indicates the point of attachment;
n is an integer selected from 1-5;
R₁ and R₂ are independently selected from hydrogen or (C₁-C₁₂)-alkyl, or R₁ and R₂ can optionally form an unsubstituted or substituted (C₃-C₇) cycloalkyl ring; and
R₃ is hydrogen or (C₁-C₁₂)-alkyl;
with the proviso that A is not a methyl group.

In another embodiment of the second aspect, the present invention provides compounds of formula 1 represented by compounds of formula 1a, wherein

B and A are as defined in the second aspect of the invention;

### Z is

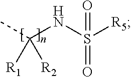

[symbol]

indicates the point of attachment;
n is an integer selected from 1-5;
R₁ and R₂ are independently selected from hydrogen or (C₁-C₁₂)-alkyl, or R₁ and R₂ can optionally form an unsubstituted or substituted (C₃-C₇) cycloalkyl ring; and
R₅ is selected from hydrogen, (C₁-C₁₂)-alkyl, CF₃, (C₃-C₇)-cycloalkyl, aryl or heterocyclyl;
with the proviso that A is not a methyl group.

In another embodiment of the second aspect, the present invention provides compounds of formula 1 represented by compounds of formula 1a, wherein

B and A are as defined in the second aspect of the invention;

### Z is

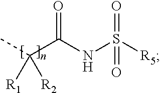

[symbol]

indicates the point of attachment;
n is an integer selected from 1-5;
R₁ and R₂ are independently selected from hydrogen or (C₁-C₁₂)-alkyl, or R₁ and R₂ can optionally form an unsubstituted or substituted (C₃-C₇) cycloalkyl ring; and
R₅ is selected from hydrogen, (C₁-C₁₂)-alkyl, CF₃, (C₃-C₇)-cycloalkyl, aryl or heterocyclyl;
with the proviso that A is not a methyl group.

In another embodiment of the second aspect, the present invention provides compounds of formula 1 represented by compounds of formula 1a, wherein

B and A are as defined in the second aspect of the invention;

### Z is

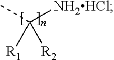

[symbol]

indicates the point of attachment;
n is an integer selected from 1-5;
R₁ and R₂ are independently selected from hydrogen or (C₁-C₁₂)-alkyl, or R₁ and R₂ can optionally form an unsubstituted or substituted (C₃-C₇) cycloalkyl ring; with the proviso that A is not a methyl group.

In another embodiment of the second aspect, the present invention provides compounds of formula 1 represented by compounds of formula 1a, wherein

B and A are as defined in the second aspect of the invention;

### Z is

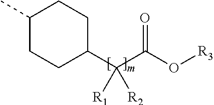

[symbol]

indicates the point of attachment;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring; and
R₃ is hydrogen or (C₁-C₁₂)-alkyl;
with the proviso that A is not a methyl group.

In another embodiment of the second aspect, the present invention provides compounds of formula 1 represented by compounds of formula 1a, wherein

B and A are as defined in the second aspect of the invention;

### Z is

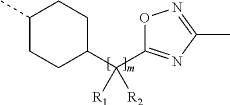

[symbol]

indicates the point of attachment;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring;
with the proviso that A is not a methyl group.

In another embodiment of the second aspect, the present invention provides compounds of formula 1 represented by compounds of formula 1a, wherein

B and A are as defined in the second aspect of the invention;

### Z is

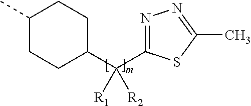

[symbol]

indicates the point of attachment;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring;
with the proviso that A is not a methyl group.

In another embodiment of the second aspect, the present invention provides compounds of formula 1 represented by compounds of formula 1a, wherein

B and A are as defined in the second aspect of the invention;

### Z is

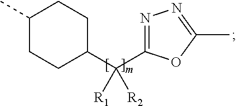

[symbol]

indicates the point of attachment;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring;
with the proviso that A is not a methyl group.

In another embodiment of the second aspect, the present invention provides compounds of formula 1 represented by compounds of formula 1a, wherein

B and A are as defined in the second aspect of the invention;

### Z is

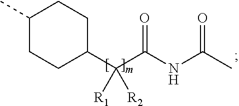

[symbol]

indicates the point of attachment;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring; with the proviso that A is not a methyl group.

In another embodiment of the second aspect, the present invention provides compounds of formula 1 represented by compounds of formula 1a, wherein

B and A are as defined in the second aspect of the invention;

### Z is

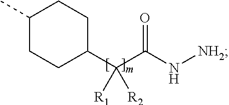

[symbol]

indicates the point of attachment;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring;
with the proviso that A is not a methyl group.

In another embodiment of the second aspect, the present invention provides compounds of formula 1 represented by compounds of formula 1a, wherein

B and A are as defined in the second aspect of the invention;

### Z is

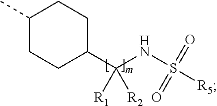

[symbol]

indicates the point of attachment;
m is 0 or 1:
R₁ and R₂ are independently selected from hydrogen or (C₁-C₁₂)-alkyl, or R₁ and R₂ can optionally form an unsubstituted or substituted (C₃-C₇) cycloalkyl ring; and
R₅ is selected from hydrogen, (C₁-C₁₂)-alkyl, CF₃, (C₃-C₇)-cycloalkyl, aryl or heterocyclyl;
with the proviso that A is not a methyl group.

In another embodiment of the second aspect, the present invention provides compounds of formula 1 represented by compounds of formula 1a, wherein

B and A are as defined in the second aspect of the invention;

### Z is

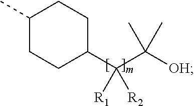

[symbol]

indicates the point of attachment;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring;
with the proviso that A is not a methyl group.

In another embodiment of the second aspect, the present invention provides compounds of formula 1 represented by compounds of formula 1a, wherein

B and A are as defined in the second aspect of the invention;

### Z is

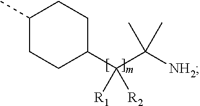

[symbol]

indicates the point of attachment;
m is 0 or 1:
R₁ and R₂ are independently selected from hydrogen or (C₁-C₁₂)-alkyl, or R₁ and R₂ can optionally form an unsubstituted or substituted (C₃-C₇) cycloalkyl ring;
with the proviso that A is not a methyl group.

In another embodiment of the second aspect, the present invention provides compounds of formula 1 represented by compounds of formula 1a, wherein

B and A are as defined in the second aspect of the invention;

### Z is

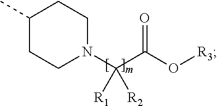

[symbol]

indicates the point of attachment;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring; and
R₃ is hydrogen or (C₁-C₁₂)-alkyl;
with the proviso that A is not a methyl group.

In another embodiment of the second aspect, the present invention provides compounds of formula 1 represented by compounds of formula 1a, wherein

B and A are as defined in the second aspect of the invention;

### Z is

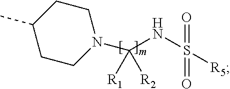

[symbol]

indicates the point of attachment;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring; and
R₅ is selected from hydrogen, (C₁-C₁₂)-alkyl, CF₃, (C₃-C₇)-cycloalkyl, aryl or heterocyclyl;
with the proviso that A is not a methyl group.

In another embodiment of the second aspect, the present invention provides compounds of formula 1 represented by compounds of formula 1a, wherein

B and A are as defined in the second aspect of the invention;

### Z is

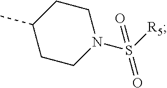

[symbol]

indicates the point of attachment;
m is 0 or 1; and
R₅ is selected from hydrogen, (C₁-C₁₂)-alkyl, CF₃, (C₃-C₇)-cycloalkyl, aryl or heterocyclyl;
with the proviso that A is not a methyl group.

In another embodiment of the second aspect, the present invention provides compounds of formula 1 represented by compounds of formula 1a, wherein

B and A are as defined in the second aspect of the invention;

### Z is

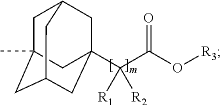

[symbol]

indicates the point of attachment;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring; and
R₃ is hydrogen or (C₁-C₁₂)-alkyl;
with the proviso that A is not a methyl group.

In a third aspect, the present invention provides compounds of formula 1 represented by compounds of formula 1a; wherein,

### B is

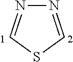

wherein 1 and 2 are the points of attachment of B to phenyl and to Z respectively;
Z is selected from:

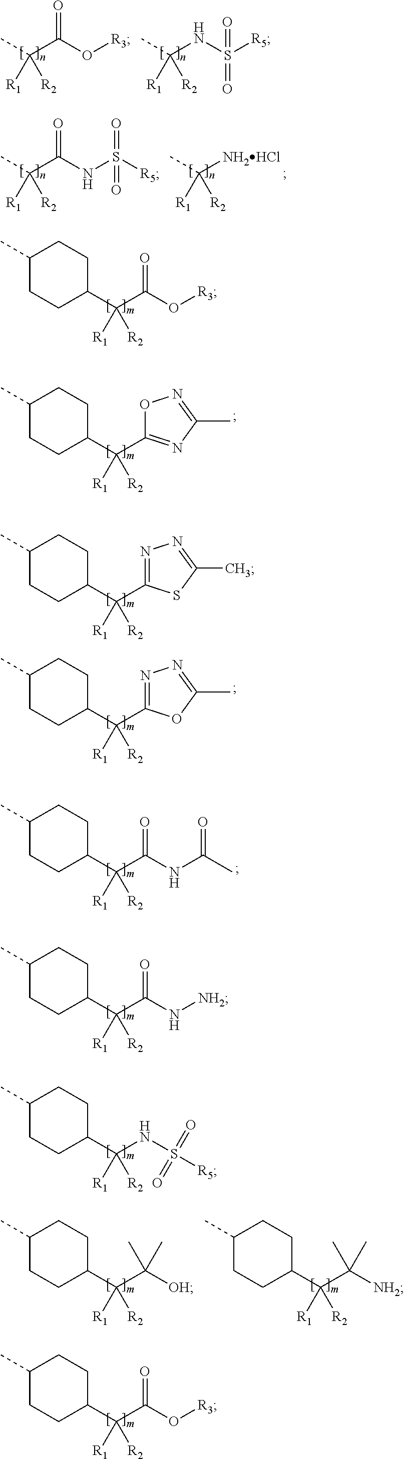

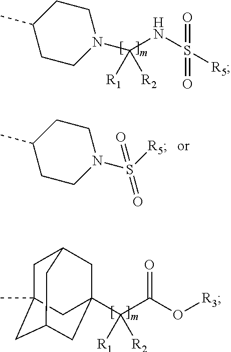

[symbol]

indicates the point of attachment;
n is an integer selected from 1-5;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring;
R₃ is hydrogen or (C₁-C₁₂)-alkyl;
R₅ is selected from hydrogen, (C₁-C₁₂)-alkyl, CF₃, (C₃-C₇)-cycloalkyl, aryl, or heterocyclyl; and
A is selected from (C₁-C₁₂)-alkyl, (C₃-C₁₂)-cycloalkyl, aryl or heterocyclyl;
wherein,
(C₁-C₁₂)-alkyl is unsubstituted or substituted with one or more groups selected from halogen, hydroxy, (C₁-C₁₂)-alkoxy, cyano, (C₃-C₁₂)-cycloalkyl, aryl or heterocyclyl;
(C₃-C₁₂)-cycloalkyl is unsubstituted or substituted with one or more groups selected from halogen, hydroxy, (C₁-C₁₂)-alkoxy, cyano, aryl or heterocyclyl;
aryl is unsubstituted or substituted with one or more groups selected from halogen, hydroxy, (C₁-C₁₂)-alkoxy, cyano, (C₁-C₁₂)-alkyl, OCF₃, CF₃, (C₃-C₁₂)-cycloalkyl, aryl, aryloxy, heterocyclyl or O-heterocyclyl; or aryl may be fused with an unsubstituted or substituted 5 or 6-membered cycloalkyl ring optionally containing one or more heteroatoms selected from O, N or S;
heterocyclyl is unsubstituted or substituted with one or more groups selected from halogen, hydroxy, (C₁-C₁₂)-alkoxy, cyano, (C₁-C₁₂)-alkyl, (C₃-C₁₂)-cycloalkyl, aryl, aryloxy, heterocyclyl or O-heterocyclyl;
with the proviso that A is not a methyl group.

In a fourth aspect, the present invention provides compounds of formula 1 represented by compounds of formula 1a; wherein,

### B is

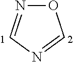

wherein 1 and 2 are the points of attachment of B to phenyl and to Z respectively;
Z is selected from:

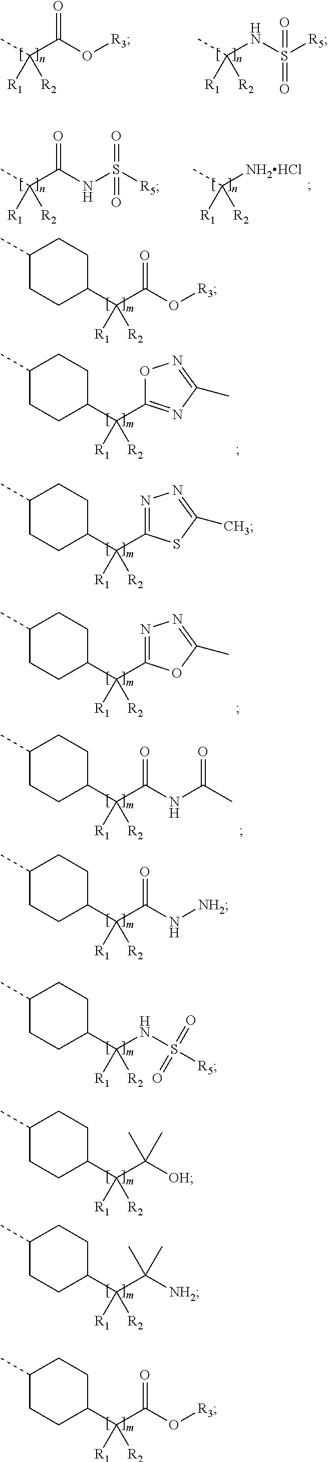

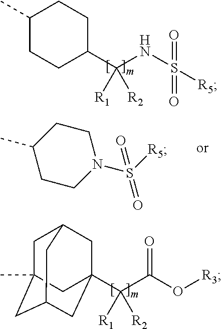

[symbol]

indicates the point of attachment;
n is an integer selected from 1-5;
m is 0 or 1:
R₁ and R₂ are independently selected from hydrogen or (C₁-C₁₂)-alkyl, or R₁ and R₂ can optionally form an unsubstituted or substituted (C₃-C₇) cycloalkyl ring;
R₃ is hydrogen or (C₁-C₁₂)-alkyl;
R₅ is selected from hydrogen, (C₁-C₁₂)-alkyl, CF₃, (C₃-C₇)-cycloalkyl, aryl, or heterocyclyl; and
A is selected from (C₁-C₁₂)-alkyl, (C₃-C₁₂)-cycloalkyl, aryl or heterocyclyl;
wherein,
(C₁-C₁₂)-alkyl is unsubstituted or substituted with one or more groups selected from halogen, hydroxy, (C₁-C₁₂)-alkoxy, cyano, (C₃-C₁₂)-cycloalkyl, aryl or heterocyclyl;
(C₃-C₁₂)-cycloalkyl is unsubstituted or substituted with one or more groups selected from halogen, hydroxy, (C₁-C₁₂)-alkoxy, cyano, aryl or heterocyclyl;
aryl is unsubstituted or substituted with one or more groups selected from halogen, hydroxy, (C₁-C₁₂)-alkoxy, cyano, (C₁-C₁₂)-alkyl, OCF₃, CF₃, (C₃-C₁₂)-cycloalkyl, aryl, aryloxy, heterocyclyl or O-heterocyclyl; or aryl may be fused with an unsubstituted or substituted 5 or 6-membered cycloalkyl ring optionally containing heteroatoms selected from O, N and S;
heterocyclyl is unsubstituted or substituted with one or more groups selected from halogen, hydroxy, (C₁-C₁₂)-alkoxy, cyano, (C₁-C₁₂)-alkyl, (C₃-C₁₂)-cycloalkyl, aryl, aryloxy, heterocyclyl or O-heterocyclyl;
with the proviso that A is not a methyl group.

In a fifth aspect, the present invention provides compounds of formula 1 represented by compounds of formula 1a; wherein,

### B is

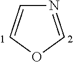

wherein 1 and 2 are the points of attachment of B to phenyl and to Z respectively;
Z is selected from:

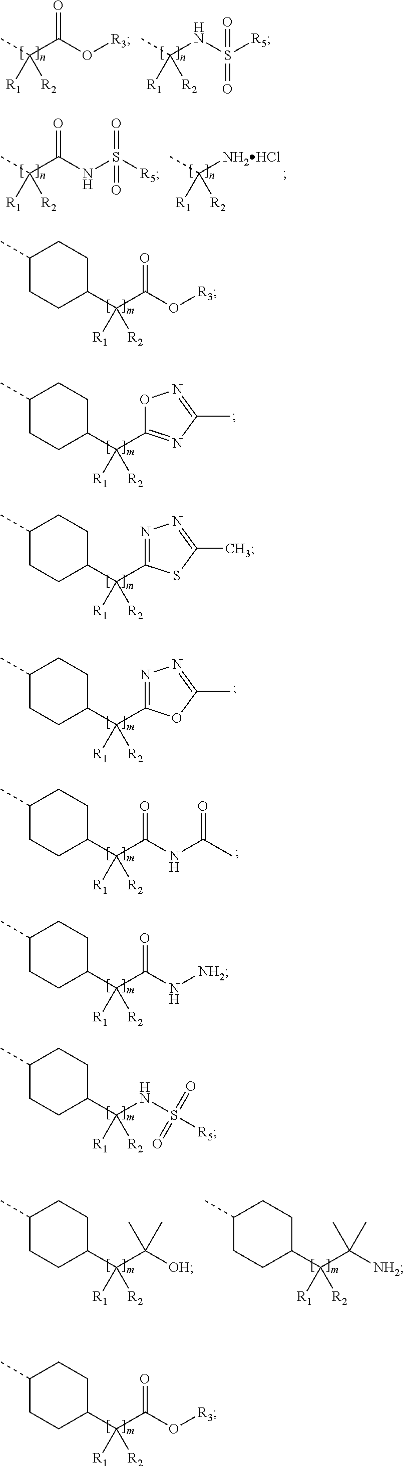

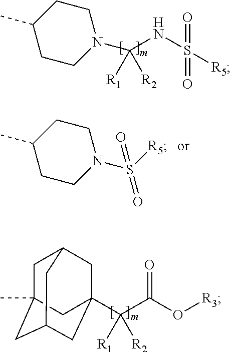

[symbol]

indicates the point of attachment;
n is an integer selected from 1-5;
m is 0 or 1:
R₁ and R₂ are independently selected from hydrogen or (C₁-C₁₂)-alkyl, or R₁ and R₂ can optionally form an unsubstituted or substituted (C₃-C₇) cycloalkyl ring;
R₃ is hydrogen or (C₁-C₁₂)-alkyl;
R₅ is selected from hydrogen, (C₁-C₁₂)-alkyl, CF₃, (C₃-C₇)-cycloalkyl, aryl, or heterocyclyl; and
A is selected from (C₁-C₁₂)-alkyl, (C₃-C₁₂)-cycloalkyl, aryl or heterocyclyl;
wherein,
(C₁-C₁₂)-alkyl is unsubstituted or substituted with one or more groups selected from halogen, hydroxy, (C₁-C₁₂)-alkoxy, cyano, (C₃-C₁₂)-cycloalkyl, aryl or heterocyclyl;
(C₃-C₁₂)-cycloalkyl is unsubstituted or substituted with one or more groups selected from halogen, hydroxy, (C₁-C₁₂)-alkoxy, cyano, aryl or heterocyclyl;
aryl is unsubstituted or substituted with one or more groups selected from halogen, hydroxy, (C₁-C₁₂)-alkoxy, cyano, (C₁-C₁₂)-alkyl, OCF₃, CF₃, (C₃-C₁₂)-cycloalkyl, aryl, aryloxy, heterocyclyl or O-heterocyclyl, or aryl may be fused with an unsubstituted or substituted 5 or 6-membered cycloalkyl ring optionally containing one or more heteroatoms selected from O, N or S;
heterocyclyl is unsubstituted or substituted with one or more groups selected from halogen, hydroxy, (C₁-C₁₂)-alkoxy, cyano, (C₁-C₁₂)-alkyl, (C₃-C₁₂)-cycloalkyl, aryl, aryloxy, heterocyclyl or O-heterocyclyl;
with the proviso that A is not a methyl group.

In a sixth aspect, the present invention provides compounds of formula 1 represented by compounds of formula 1b,

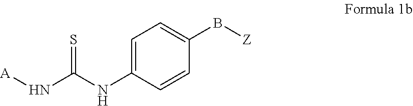

in all their stereoisomeric and tautomeric forms; and their pharmaceutically acceptable salts, solvates, polymorphs, prodrugs, carboxylic acid isosteres and N-oxides;
wherein,
Z is selected from:

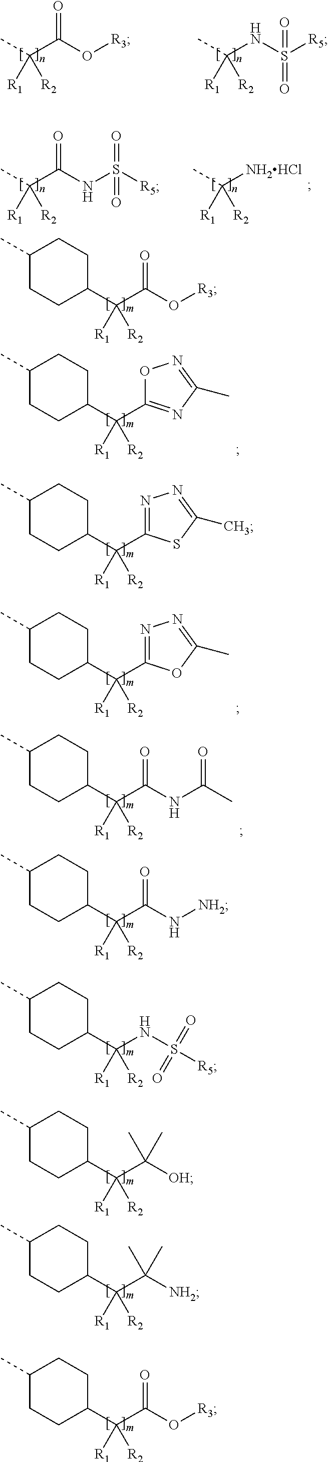

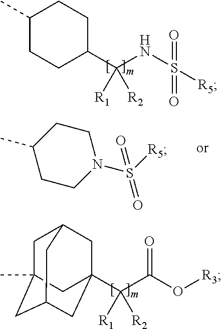

[symbol]

indicates the point of attachment;
n is an integer selected from 1-5;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring;
R₃ is hydrogen or (C₁-C₁₂)-alkyl;
R₅ is selected from hydrogen, (C₁-C₁₂)-alkyl, CF₃, (C₃-C₇)-cycloalkyl, aryl, or heterocyclyl;
B is a 5-membered heteroaryl ring represented by any one of the general structures (i) to (x);

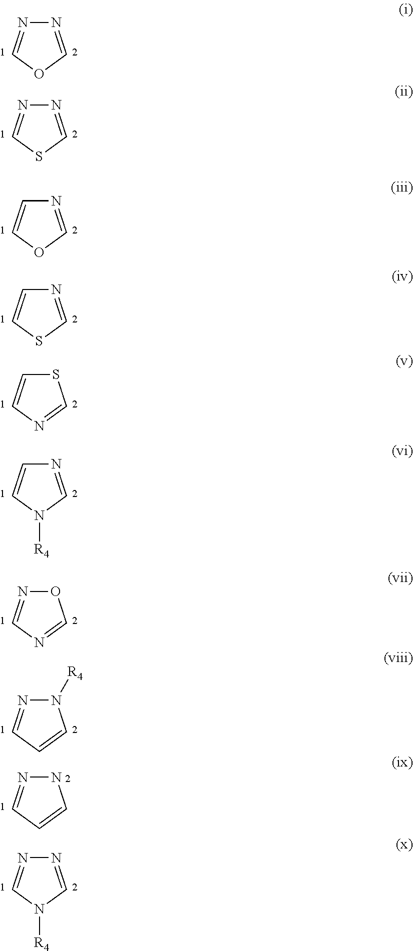

wherein 1 and 2 are the points of attachment of B to phenyl and to Z respectively and R₄ is selected from hydrogen, (C₁-C₁₂)-alkyl or aryl; or B is a 6-membered heteroaryl ring containing 1 or 2 N-atoms, wherein the 6-membered heteroaryl ring may be unsubstituted or substituted with one or more groups selected from halogen, hydroxy, (C₁-C₁₂)-alkoxy, cyano, nitro, (C₁-C₁₂)-alkyl, (C₂-C₁₂)-alkenyl, (C₂-C₁₂)-alkynyl, (C₃-C₁₂)-cycloalkyl, aryl, aryloxy, heterocyclyl or O-heterocyclyl; and
A is selected from (C₁-C₁₂)-alkyl, (C₃-C₁₂)-cycloalkyl, aryl or heterocyclyl;
wherein,
(C₁-C₁₂)-alkyl is unsubstituted or substituted with one or more groups selected from halogen, hydroxy, (C₁-C₁₂)-alkoxy, cyano, nitro, (C₃-C₁₂)-cycloalkyl, aryl, heterocyclyl, C(O)Rₚ, C(O)ORₚ, NRₚR_{q}, C(O)NRₚR_{q}, SRₚ, S(O)Rₚ or SO₂Rₚ;
(C₃-C₁₂)-cycloalkyl is unsubstituted or substituted with one or more groups selected from halogen, hydroxy, (C₁-C₁₂)-alkoxy, cyano, nitro, aryl, heterocyclyl, C(O)Rₚ, C(O)ORₚ, NRₚR_{q}, C(O)NRₚR_{q}, SRₚ, S(O)Rₚ or SO₂Rₚ;
aryl is unsubstituted or substituted with one or more groups selected from halogen, hydroxy, (C₁-C₁₂)-alkoxy, cyano, nitro, (C₁-C₁₂)-alkyl, OCF₃, CF₃, (C₂-C₁₂)-alkenyl, (C₂-C₁₂)-alkynyl, (C₃-C₁₂)-cycloalkyl, aryl, aryloxy, heterocyclyl, O-heterocyclyl, C(O)Rₚ, C(O)ORₚ, NRₚR_{q}, C(O)NRₚR_{q}, SRₚ, S(O)Rₚ or SO₂Rₚ or aryl may be fused with an unsubstituted or substituted 5 or 6-membered cycloalkyl ring optionally containing one or more heteroatoms selected from O, N or S;
heterocyclyl is unsubstituted or substituted with one or more groups selected from halogen, hydroxy, (C₁-C₁₂)-alkoxy, cyano, nitro, (C₁-C₁₂)-alkyl, (C₂-C₁₂)-alkenyl, (C₂-C₁₂)-alkynyl, (C₃-C₁₂)-cycloalkyl, aryl, aryloxy, heterocyclyl, O-heterocyclyl, C(O)Rₚ, C(O)ORₚ, NRₚR_{q}, C(O)NRₚR_{q}, SRₚ, S(O)Rₚ or SO₂Rₚ;
Rₚ and R_{q} are independently selected from hydrogen, (C₁-C₁₂)-alkyl, aryl, aralkyl or heterocyclyl, or Rₚ and R_{q} together with the N to which they are attached optionally form a 3 to 7 membered ring;
with the proviso that A is not a methyl group.

In a seventh aspect, the present invention provides compounds of formula 1 represented by compounds of formula 1b, wherein,

### B is

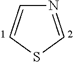

wherein 1 and 2 are the points of attachment of B to phenyl and to Z respectively;

### Z is

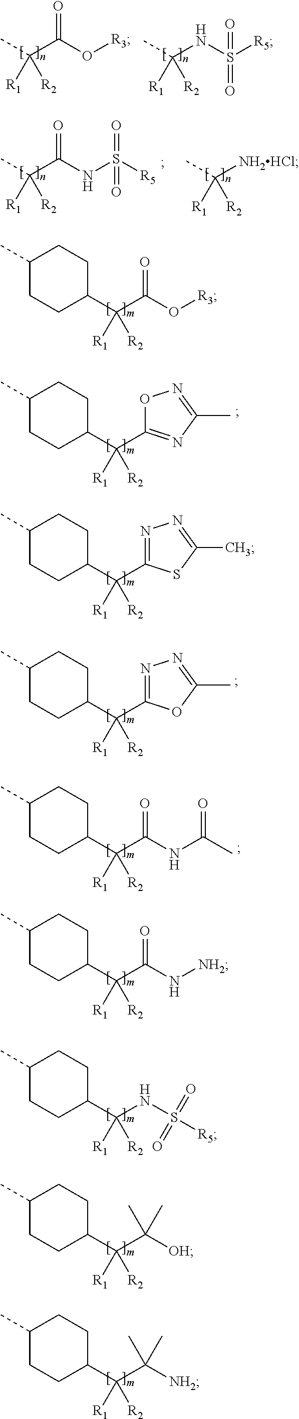

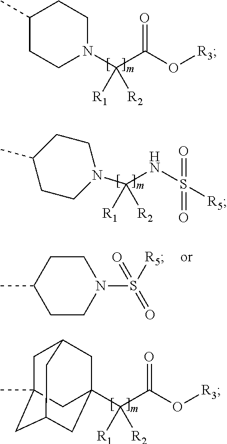

[symbol]

indicates the point of attachment;
n is an integer selected from 1-5;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring;
R₃ is hydrogen or (C₁-C₁₂)-alkyl;
R₅ is selected from hydrogen, (C₁-C₁₂)-alkyl, CF₃, (C₃-C₇)-cycloalkyl, aryl or heterocyclyl; and
A is selected from (C₁-C₁₂)-alkyl, (C₃-C₁₂)-cycloalkyl, aryl or heterocyclyl;
wherein,
(C₁-C₁₂)-alkyl is unsubstituted or substituted with one or more groups selected from halogen, hydroxy, (C₁-C₁₂)-alkoxy, cyano, (C₃-C₁₂)-cycloalkyl, aryl or heterocyclyl;
(C₃-C₁₂)-cycloalkyl is unsubstituted or substituted with one or more groups selected from halogen, hydroxy, (C₁-C₁₂)-alkoxy, cyano, aryl or heterocyclyl;
aryl is unsubstituted or substituted with one or more groups selected from halogen, hydroxy, (C₁-C₁₂)-alkoxy, cyano, (C₁-C₁₂)-alkyl, OCF₃, CF₃, (C₃-C₁₂)-cycloalkyl, aryl, aryloxy, heterocyclyl or O-heterocyclyl; or aryl may be fused with an unsubstituted or substituted 5 or 6-membered cycloalkyl ring optionally containing one or more heteroatoms selected from O, N or S;
heterocyclyl is unsubstituted or substituted with one or more groups selected from halogen, hydroxy, (C₁-C₁₂)-alkoxy, cyano, (C₁-C₁₂)-alkyl, (C₃-C₁₂)-cycloalkyl, aryl, aryloxy, heterocyclyl or O-heterocyclyl;
with the proviso that A is not a methyl group.

In an embodiment of the seventh aspect, Z is

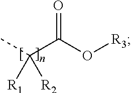

[symbol]

indicates the point of attachment;
n is an integer selected from 1-5;
R₁ and R₂ are independently selected from hydrogen or (C₁-C₁₂)-alkyl, or R₁ and R₂ can optionally form an unsubstituted or substituted (C₃-C₇) cycloalkyl ring;
R₃ is hydrogen or (C₁-C₁₂)-alkyl; and B and A are as defined above with the proviso that A is not a methyl group.

In another embodiment of the seventh aspect, Z is

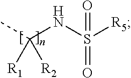

[symbol]

indicates the point of attachment;
n is an integer selected from 1-5;
R₁ and R₂ are independently selected from hydrogen or (C₁-C₁₂)-alkyl, or R₁ and R₂ can optionally form an unsubstituted or substituted (C₃-C₇) cycloalkyl ring;
R₅ is selected from hydrogen, (C₁-C₁₂)-alkyl, CF₃, (C₃-C₇)-cycloalkyl, aryl, or heterocyclyl; and B and A are as defined in the seventh aspect,
with the proviso that A is not a methyl group.

In another embodiment of the seventh aspect, Z is

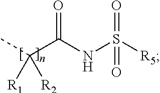

[symbol]

indicates the point of attachment;
n is an integer selected from 1-5;
R₁ and R₂ are independently selected from hydrogen or (C₁-C₁₂)-alkyl, or R₁ and R₂ can optionally form an unsubstituted or substituted (C₃-C₇) cycloalkyl ring;
R₅ is selected from hydrogen, (C₁-C₁₂)-alkyl, CF₃, (C₃-C₇)-cycloalkyl, aryl, or heterocyclyl; and B and A are as defined in the seventh aspect,
with the proviso that A is not a methyl group.

In another embodiment of the seventh aspect, Z is

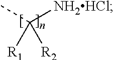

[symbol]

indicates the point of attachment;
n is an integer selected from 1-5;
R₁ and R₂ are independently selected from hydrogen or (C₁-C₁₂)-alkyl, or R₁ and R₂ can optionally form an unsubstituted or substituted (C₃-C₇) cycloalkyl ring; and B and A are as defined in the seventh aspect,
with the proviso that A is not a methyl group.

In another embodiment of the seventh aspect, Z is

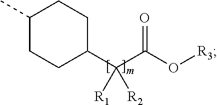

[symbol]

indicates the point of attachment;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring;
R₃ is hydrogen or (C₁-C₁₂)-alkyl; and B and A are as defined in the seventh aspect, with the proviso that A is not a methyl group.

In another embodiment of the seventh aspect, Z is

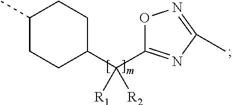

[symbol]

indicates the point of attachment;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring; and B and A are as defined in the seventh aspect,
with the proviso that A is not a methyl group.

In another embodiment of the seventh aspect, Z is

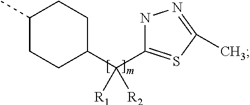

[symbol]

indicates the point of attachment;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring; and B and A are as defined in the seventh aspect,
with the proviso that A is not a methyl group.

In another embodiment of the seventh aspect, Z is

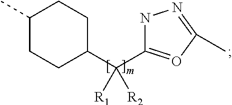

[symbol]

indicates the point of attachment;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring; and B and A are as defined in the seventh aspect,
with the proviso that A is not a methyl group.

In another embodiment of the seventh aspect, Z is

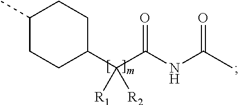

[symbol]

indicates the point of attachment;
m is 0 or 1:
R₁ and R₂ are independently selected from hydrogen or (C₁-C₁₂)-alkyl, or R₁ and R₂ can optionally form an unsubstituted or substituted (C₃-C₇) cycloalkyl ring; and B and A are as defined in the seventh aspect,
with the proviso that A is not a methyl group.

In another embodiment of the seventh aspect, Z is

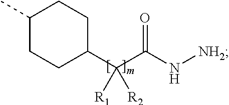

[symbol]

indicates the point of attachment;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring; and B and A are as defined in the seventh aspect,
with the proviso that A is not a methyl group.

In another embodiment of the seventh aspect, Z is

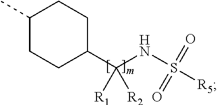

[symbol]

indicates the point of attachment;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring;

R₅ is selected from hydrogen, (C₁-C₁₂)-alkyl, CF₃, (C₃-C₇)-cycloalkyl, aryl, or heterocyclyl; and B and A are as defined in the seventh aspect,

with the proviso that A is not a methyl group.

In another embodiment of the seventh aspect, Z is

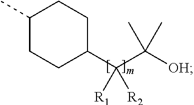

[symbol]

indicates the point of attachment;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring; and B and A are as defined in the seventh aspect,
with the proviso that A is not a methyl group.

In another embodiment of the seventh aspect, Z is

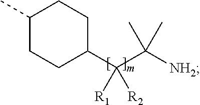

[symbol]

indicates the point of attachment;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring; and B and A are as defined in the seventh aspect,
with the proviso that A is not a methyl group.

In another embodiment of the seventh aspect, Z is

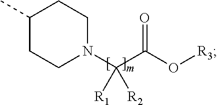

[symbol]

indicates the point of attachment;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring;

R₃ is hydrogen or (C₁-C₁₂)-alkyl; and B and A are as defined in the seventh aspect,

with the proviso that A is not a methyl group.

In another embodiment of the seventh aspect, Z is

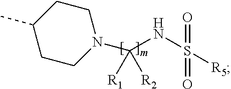

[symbol]

indicates the point of attachment;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring;

R₅ is selected from hydrogen, (C₁-C₁₂)-alkyl, CF₃, (C₃-C₇)-cycloalkyl, aryl, or heterocyclyl; and B and A are as defined in the seventh aspect,

with the proviso that A is not a methyl group.

In another embodiment of the seventh aspect, Z is

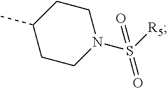

[symbol]

indicates the point of attachment;

R₅ is selected from hydrogen, (C₁-C₁₂)-alkyl, CF₃, (C₃-C₇)-cycloalkyl, aryl, and heterocyclyl; and B and A are as defined in the seventh aspect,

with the proviso that A is not a methyl group.

In another embodiment of the seventh aspect, Z is

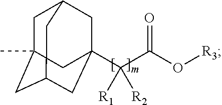

[symbol]

indicates the point of attachment;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring;
R₃ is hydrogen or (C₁-C₁₂)-alkyl; and B and A are as defined in the seventh aspect,
with the proviso that A is not a methyl group.

In an eighth aspect, the present invention provides compounds of formula 1 represented by compounds of formula 1b; wherein,

### B is

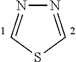

wherein 1 and 2 are the points of attachment of B to phenyl and to Z respectively;
Z is selected from:

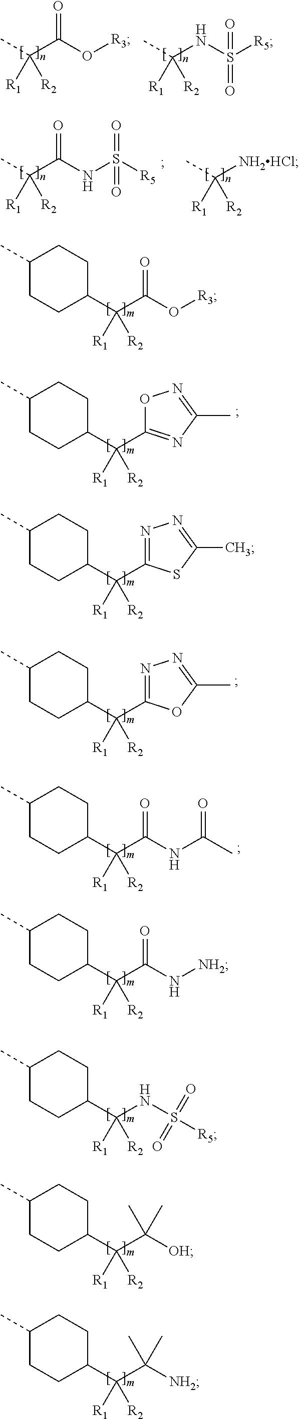

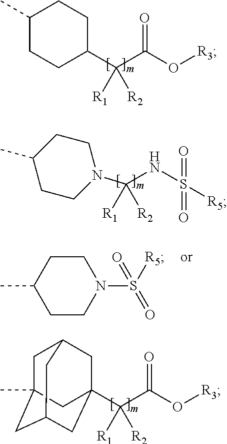

[symbol]

indicates the point of attachment;
n is an integer selected from 1-5;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring;
R₃ is hydrogen or (C₁-C₁₂)-alkyl;
R₅ is selected from hydrogen, (C₁-C₁₂)-alkyl, CF₃, (C₃-C₇)-cycloalkyl, aryl, or heterocyclyl; and
A is selected from (C₁-C₁₂)-alkyl, (C₃-C₁₂)-cycloalkyl, aryl or heterocyclyl;
wherein,
(C₁-C₁₂)-alkyl is unsubstituted or substituted with one or more groups selected from halogen, hydroxy, (C₁-C₁₂)-alkoxy, cyano, (C₃-C₁₂)-cycloalkyl, aryl or heterocyclyl;
(C₃-C₁₂)-cycloalkyl is unsubstituted or substituted with one or more groups selected from halogen, hydroxy, (C₁-C₁₂)-alkoxy, cyano, aryl or heterocyclyl;
aryl is unsubstituted or substituted with one or more groups selected from halogen, hydroxy, (C₁-C₁₂)-alkoxy, cyano, (C₁-C₁₂)-alkyl, OCF₃, CF₃, (C₃-C₁₂)-cycloalkyl, aryl, aryloxy, heterocyclyl or O-heterocyclyl; or aryl may be fused with an unsubstituted or substituted 5 or 6-membered cycloalkyl ring optionally containing one or more heteroatoms selected from O, N or S;
heterocyclyl is unsubstituted or substituted with one or more groups selected from halogen, hydroxy, (C₁-C₁₂)-alkoxy, cyano, (C₁-C₁₂)-alkyl, (C₃-C₁₂)-cycloalkyl, aryl, aryloxy, heterocyclyl or O-heterocyclyl;
with the proviso that A is not a methyl group.

In a ninth aspect, the present invention provides compounds of formula 1 represented by compounds of formula 1b; wherein,

### B is

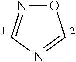

wherein 1 and 2 are the points of attachment of B to phenyl and to Z respectively; Z is selected from:

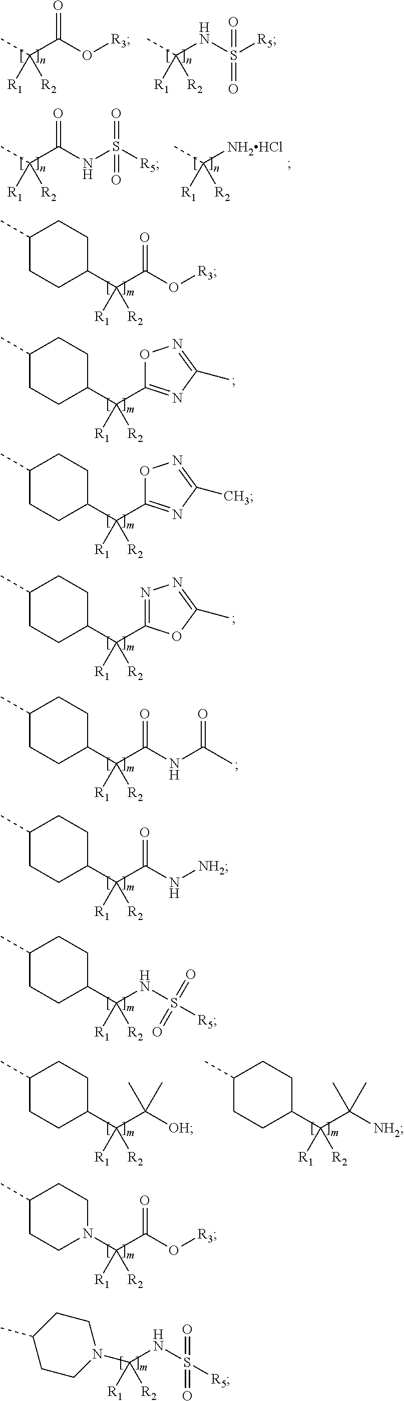

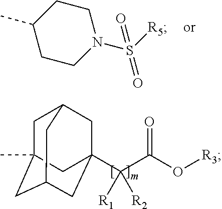

[symbol]

indicates the point of attachment;
n is an integer selected from 1-5;
m is 0 or 1:
R₁ and R₂ are independently selected from hydrogen or (C₁-C₁₂)-alkyl, or R₁ and R₂ can optionally form an unsubstituted or substituted (C₃-C₇) cycloalkyl ring;
R₃ is hydrogen or (C₁-C₁₂)-alkyl;
R₅ is selected from hydrogen, (C₁-C₁₂)-alkyl, CF₃, (C₃-C₇)-cycloalkyl, aryl, or heterocyclyl; and
A is selected from (C₁-C₁₂)-alkyl, (C₃-C₁₂)-cycloalkyl, aryl or heterocyclyl;
wherein,
(C₁-C₁₂)-alkyl is unsubstituted or substituted with one or more groups selected from halogen, hydroxy, (C₁-C₁₂)-alkoxy, cyano, (C₃-C₁₂)-cycloalkyl, aryl or heterocyclyl;
(C₃-C₁₂)-cycloalkyl is unsubstituted or substituted with one or more groups selected from halogen, hydroxy, (C₁-C₁₂)-alkoxy, cyano, aryl or heterocyclyl;
aryl is unsubstituted or substituted with one or more groups selected from halogen, hydroxy, (C₁-C₁₂)-alkoxy, cyano, (C₁-C₁₂)-alkyl, OCF₃, CF₃, (C₃-C₁₂)-cycloalkyl, aryl, aryloxy, heterocyclyl or O-heterocyclyl, or aryl may be fused with an unsubstituted or substituted 5 or 6-membered cycloalkyl ring optionally containing one or more heteroatoms selected from O, N or S;
heterocyclyl is unsubstituted or substituted with one or more groups selected from halogen, hydroxy, (C₁-C₁₂)-alkoxy, cyano, (C₁-C₁₂)-alkyl, (C₃-C₁₂)-cycloalkyl, aryl, aryloxy, heterocyclyl or O-heterocyclyl;
with the proviso that A is not a methyl group.

In a tenth aspect, the present invention provides compounds of formula 1 represented by compounds of formula 1b; wherein,

### B is

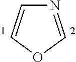

wherein 1 and 2 are the points of attachment of B to phenyl and to Z respectively; Z is selected from:

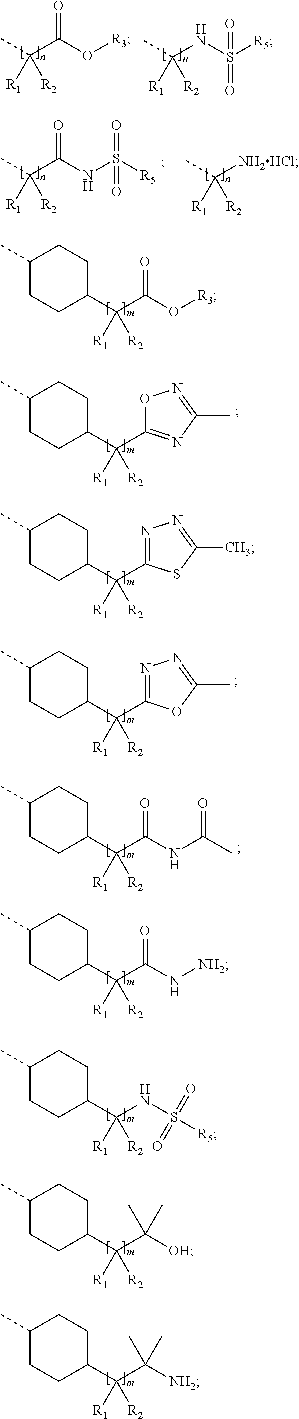

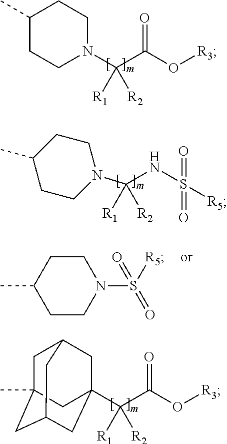

[symbol]

indicates the point of attachment;
n is an integer selected from 1-5;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring;
R₃ is hydrogen or (C₁-C₁₂)-alkyl;
R₅ is selected from hydrogen, (C₁-C₁₂)-alkyl, CF₃, (C₃-C₇)-cycloalkyl, aryl, or heterocyclyl; and
A is selected from (C₁-C₁₂)-alkyl, (C₃-C₁₂)-cycloalkyl, aryl or heterocyclyl;
wherein,
(C₁-C₁₂)-alkyl is unsubstituted or substituted with one or more groups selected from halogen, hydroxy, (C₁-C₁₂)-alkoxy, cyano, (C₃-C₁₂)-cycloalkyl, aryl or heterocyclyl;
(C₃-C₁₂)-cycloalkyl is unsubstituted or substituted with one or more groups selected from halogen, hydroxy, (C₁-C₁₂)-alkoxy, cyano, aryl or heterocyclyl;
aryl is unsubstituted or substituted with one or more groups selected from halogen, hydroxy, (C₁-C₁₂)-alkoxy, cyano, (C₁-C₁₂)-alkyl, OCF₃, CF₃, (C₃-C₁₂)-cycloalkyl, aryl, aryloxy, heterocyclyl or O-heterocyclyl, or aryl may be fused with an unsubstituted or substituted 5 or 6-membered cycloalkyl ring optionally containing one or more heteroatoms selected from O, N and S;
heterocyclyl is unsubstituted or substituted with one or more groups selected from halogen, hydroxy, (C₁-C₁₂)-alkoxy, cyano, (C₁-C₁₂)-alkyl, (C₃-C₁₂)-cycloalkyl, aryl, aryloxy, heterocyclyl or O-heterocyclyl;
with the proviso that A is not a methyl group.

In an eleventh aspect, the present invention provides compounds of formula 1 represented by compounds of formula 1c,

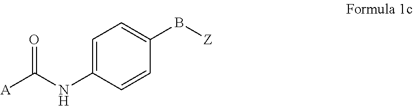

in all their stereoisomeric and tautomeric forms; and their pharmaceutically acceptable salts, solvates, polymorphs, prodrugs, carboxylic acid isosteres and N-oxides;
wherein,
Z is selected from:

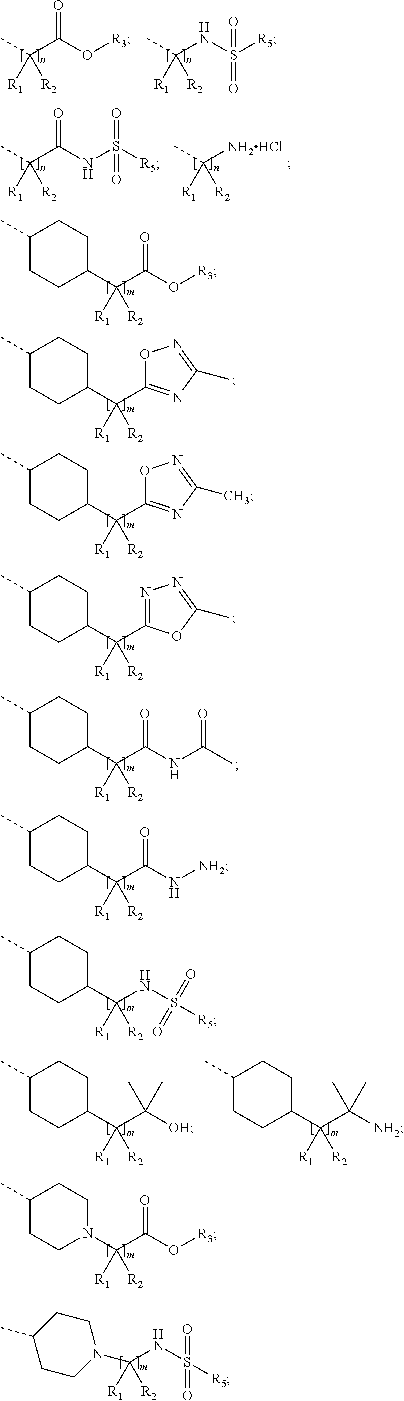

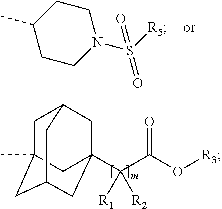

[symbol]

indicates the point of attachment;
n is an integer selected from 1-5;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring;
R₃ is hydrogen or (C₁-C₁₂)-alkyl;
R₅ is selected from hydrogen, (C₁-C₁₂)-alkyl, CF₃, (C₃-C₇)-cycloalkyl, aryl, or heterocyclyl;
B is a 5-membered heteroaryl ring represented by any one of the general structures (i) to (x);

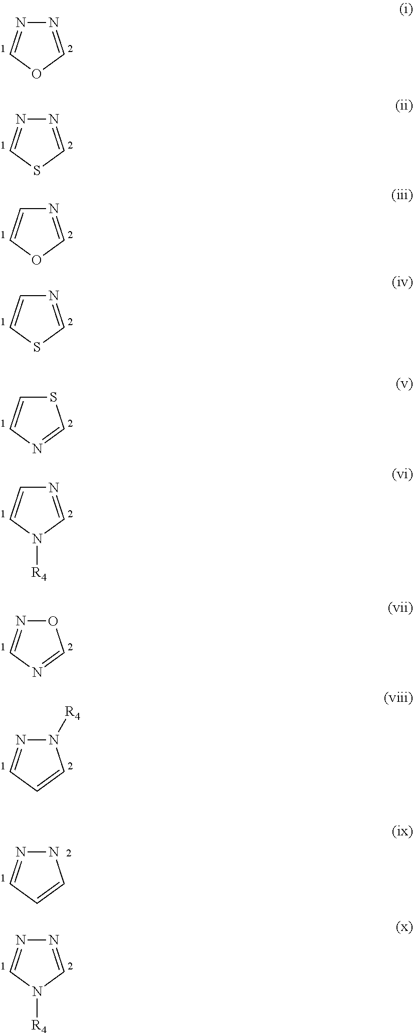

wherein 1 and 2 are the points of attachment of B to phenyl and to Z respectively and R₄ is selected from hydrogen, (C₁-C₁₂)-alkyl or aryl; or B is a 6-membered heteroaryl ring containing 1 or 2 N-atoms, wherein the 6-membered heteroaryl ring may be unsubstituted or substituted with one or more groups selected from halogen, hydroxy, (C₁-C₁₂)-alkoxy, cyano, nitro, (C₁-C₁₂)-alkyl, (C₂-C₁₂)-alkenyl, (C₂-C₁₂)-alkynyl, (C₃-C₁₂)-cycloalkyl, aryl, aryloxy, heterocyclyl or O-heterocyclyl; and
A is selected from (C₁-C₁₂)-alkyl, (C₃-C₁₂)-cycloalkyl, aryl or heterocyclyl;
wherein,
(C₁-C₁₂)-alkyl is unsubstituted or substituted with one or more groups selected from halogen, hydroxy, (C₁-C₁₂)-alkoxy, cyano, nitro, (C₃-C₁₂)-cycloalkyl, aryl, heterocyclyl,
C(O)Rₚ, C(O)ORₚ, NRₚR_{q}, C(O)NRₚR_{q}, SRₚ, S(O)Rₚ or SO₂Rₚ;
(C₃-C₁₂)-cycloalkyl is unsubstituted or substituted with one or more groups selected from halogen, hydroxy, (C₁-C₁₂)-alkoxy cyano, nitro, aryl, heterocyclyl, C(O)Rₚ, C(O)ORₚ, NRₚR_{q}, C(O)NRₚR_{q}, SRₚ, S(O)Rₚ or SO₂Rₚ;
aryl is unsubstituted or substituted with one or more groups selected from halogen, hydroxy, (C₁-C₁₂)-alkoxy, cyano, nitro, (C₁-C₁₂)-alkyl, OCF₃, CF₃, (C₂-C₁₂)-alkenyl, (C₂-C₁₂)-alkynyl, (C₃-C₁₂)-cycloalkyl, aryl, aryloxy, heterocyclyl, O-heterocyclyl, C(O)Rₚ, C(O)ORₚ, NRₚR_{q}, C(O)NRₚR_{q}, SRₚ, S(O)Rₚ or SO₂Rₚ; or aryl may be fused with an unsubstituted or substituted 5 or 6-membered cycloalkyl ring optionally containing one or more heteroatoms selected from O, N or S;
heterocyclyl is unsubstituted or substituted with one or more groups selected from halogen, hydroxy, (C₁-C₁₂)-alkoxy, cyano, nitro, (C₁-C₁₂)-alkyl, (C₂-C₁₂)-alkenyl, (C₂-C₁₂)-alkynyl, (C₃-C₁₂)-cycloalkyl, aryl, aryloxy, heterocyclyl, O-heterocyclyl, C(O)Rₚ, C(O)ORₚ, NRₚR_{q}, C(O)NRₚR_{q}, SRₚ, S(O)Rₚ or SO₂Rₚ;
Rₚ and R_{q} are independently selected from hydrogen, (C₁-C₁₂)-alkyl, aryl, aralkyl or heterocyclyl, or Rₚ and R_{q} together with the N to which they are attached optionally form a 3 to 7 membered ring;
with the proviso that A is not a methyl group.

In a twelfth aspect, the present invention provides compounds of formula 1 represented by compounds of formula 1c, wherein,

### B is

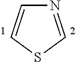

wherein 1 and 2 are the points of attachment of B to phenyl and to Z respectively;

### Z is

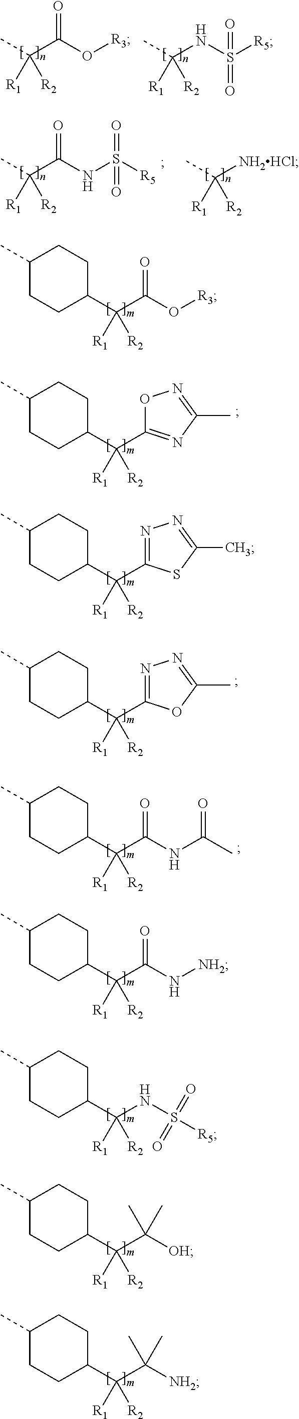

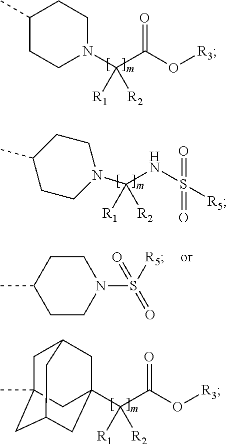

[symbol]

indicates the point of attachment;
n is an integer selected from 1-5;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring;
R₃ is hydrogen or (C₁-C₁₂)-alkyl;
R₅ is selected from hydrogen, (C₁-C₁₂)-alkyl, CF₃, (C₃-C₇)-cycloalkyl, aryl or heterocyclyl; and
A is selected from (C₁-C₁₂)-alkyl, (C₃-C₁₂)-cycloalkyl, aryl or heterocyclyl;
wherein,
(C₁-C₁₂)-alkyl is unsubstituted or substituted with one or more groups selected from halogen, hydroxy, (C₁-C₁₂)-alkoxy, cyano, (C₃-C₁₂)-cycloalkyl, aryl or heterocyclyl;
(C₃-C₁₂)-cycloalkyl is unsubstituted or substituted with one or more groups selected from halogen, hydroxy, (C₁-C₁₂)-alkoxy, cyano, aryl or heterocyclyl;
aryl is unsubstituted or substituted with one or more groups selected from halogen, hydroxy, (C₁-C₁₂)-alkoxy, cyano, (C₁-C₁₂)-alkyl, OCF₃, CF₃, (C₃-C₁₂)-cycloalkyl, aryl, aryloxy, heterocyclyl or O-heterocyclyl; or aryl may be fused with an unsubstituted or substituted 5 or 6-membered cycloalkyl ring optionally containing one or more heteroatoms selected from O, N or S;
heterocyclyl is unsubstituted or substituted with one or more groups selected from halogen, hydroxy, (C₁-C₁₂)-alkoxy, cyano, (C₁-C₁₂)-alkyl, (C₃-C₁₂)-cycloalkyl, aryl, aryloxy, heterocyclyl or O-heterocyclyl;
with the proviso that A is not a methyl group.

In an embodiment of the twelfth aspect, Z is

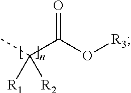

[symbol]

indicates the point of attachment;
n is an integer selected from 1-5;
R₁ and R₂ are independently selected from hydrogen or (C₁-C₁₂)-alkyl, or R₁ and R₂ can optionally form an unsubstituted or substituted (C₃-C₇) cycloalkyl ring;
R₃ is hydrogen or (C₁-C₁₂)-alkyl; and B and A are as defined in the twelfth aspect,
with the proviso that A is not a methyl group.

In another embodiment of the twelfth aspect, Z is

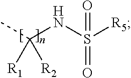

[symbol]

indicates the point of attachment;
n is an integer selected from 1-5;
R₁ and R₂ are independently selected from hydrogen or (C₁-C₁₂)-alkyl, or R₁ and R₂ can optionally form an unsubstituted or substituted (C₃-C₇) cycloalkyl ring;
R₅ is selected from hydrogen, (C₁-C₁₂)-alkyl, CF₃, (C₃-C₇)-cycloalkyl, aryl, or heterocyclyl; and B and A are as defined in the twelfth aspect,
with the proviso that A is not a methyl group.

In another embodiment of the twelfth aspect, Z is

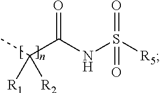

[symbol]

indicates the point of attachment;
n is an integer selected from 1-5;
R₁ and R₂ are independently selected from hydrogen or (C₁-C₁₂)-alkyl, or R₁ and R₂ can optionally form an unsubstituted or substituted (C₃-C₇) cycloalkyl ring;
R₅ is selected from hydrogen, (C₁-C₁₂)-alkyl, CF₃, (C₃-C₇)-cycloalkyl, aryl, or heterocyclyl; and B and A are as defined in the twelfth aspect,
with the proviso that A is not a methyl group.

In another embodiment of the twelfth aspect, Z is

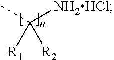

[symbol]

indicates the point of attachment;
n is an integer selected from 1-5;
R₁ and R₂ are independently selected from hydrogen or (C₁-C₁₂)-alkyl, or R₁ and R₂ can optionally form an unsubstituted or substituted (C₃-C₇) cycloalkyl ring; and B and A are as defined in the twelfth aspect,
with the proviso that A is not a methyl group.

In another embodiment of the twelfth aspect, Z is

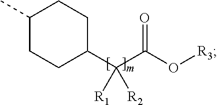

[symbol]

indicates the point of attachment;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring;
R₃ is hydrogen or (C₁-C₁₂)-alkyl; and B and A are as defined in the twelfth aspect,
with the proviso that A is not a methyl group.

In another embodiment of the twelfth aspect, Z is

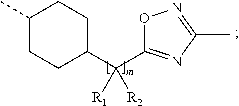

[symbol]

indicates the point of attachment;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring; and B and A are as defined in the twelfth aspect,
with the proviso that A is not a methyl group.

In another embodiment of the twelfth aspect, Z is

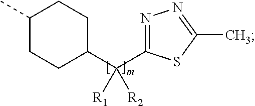

[symbol]

indicates the point of attachment;
m is 0 or 1:
R₁ and R₂ are independently selected from hydrogen or (C₁-C₁₂)-alkyl, or R₁ and R₂ can optionally form an unsubstituted or substituted (C₃-C₇) cycloalkyl ring; and B and A are as defined in the twelfth aspect,
with the proviso that A is not a methyl group.

In another embodiment of the twelfth aspect, Z is

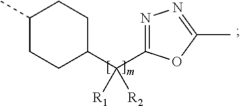

[symbol]

indicates the point of attachment;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring; and B and A are as defined in the twelfth aspect,
with the proviso that A is not a methyl group.

In another embodiment of the twelfth aspect, Z is

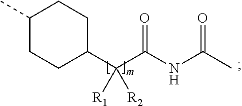

[symbol]

indicates the point of attachment;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring; and B and A are as defined in the twelfth aspect,
with the proviso that A is not a methyl group.

In another embodiment of the twelfth aspect, Z is

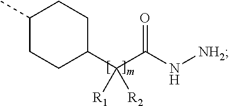

[symbol]

indicates the point of attachment;
m is 0 or 1:
R₁ and R₂ are independently selected from hydrogen or (C₁-C₁₂)-alkyl, or R₁ and R₂ can optionally form an unsubstituted or substituted (C₃-C₇) cycloalkyl ring; and B and A are as defined in the twelfth aspect,
with the proviso that A is not a methyl group.

In another embodiment of the twelfth aspect, Z is

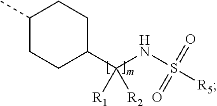

[symbol]

indicates the point of attachment;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring;
R₅ is selected from hydrogen, (C₁-C₁₂)-alkyl, CF₃, (C₃-C₇)-cycloalkyl, aryl, or heterocyclyl; and B and A are as defined in the twelfth aspect,
with the proviso that A is not a methyl group.

In another embodiment of the twelfth aspect, Z is

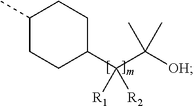

[symbol]

indicates the point of attachment;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring; and B and A are as defined in the twelfth aspect,
with the proviso that A is not a methyl group.

In another embodiment of the twelfth aspect, Z is

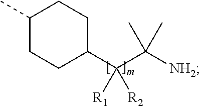

[symbol]

indicates the point of attachment;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring; and B and A are as defined in the twelfth aspect,
with the proviso that A is not a methyl group.

In another embodiment of the twelfth aspect, Z is

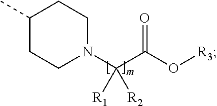

[symbol]

indicates the point of attachment;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring;

R₃ is hydrogen or (C₁-C₁₂)-alkyl; and B and A are as defined in the twelfth aspect,

with the proviso that A is not a methyl group.

In another embodiment of the twelfth aspect, Z is

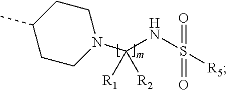

[symbol]

indicates the point of attachment;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring;
R₅ is selected from hydrogen, (C₁-C₁₂)-alkyl, CF₃, (C₃-C₇)-cycloalkyl, aryl, or heterocyclyl; and B and A are as defined in the twelfth aspect,
with the proviso that A is not a methyl group.

In another embodiment of the twelfth aspect, Z is

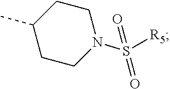

[symbol]

indicates the point of attachment;
R₅ is selected from hydrogen, (C₁-C₁₂)-alkyl, CF₃, (C₃-C₇)-cycloalkyl, aryl, and heterocyclyl; and B and A are as defined in the twelfth aspect,
with the proviso that A is not a methyl group.

In another embodiment of the twelfth aspect, Z is

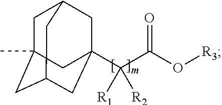

[symbol]

indicates the point of attachment;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring;
R₃ is hydrogen or (C₁-C₁₂)-alkyl; and B and A are as defined in the twelfth aspect,
with the proviso that A is not a methyl group.

In a thirteenth aspect, the present invention provides compounds of formula 1 represented by compounds of formula 1c; wherein,

### B is

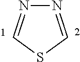

wherein 1 and 2 are the points of attachment of B to phenyl and to Z respectively;
Z is selected from:

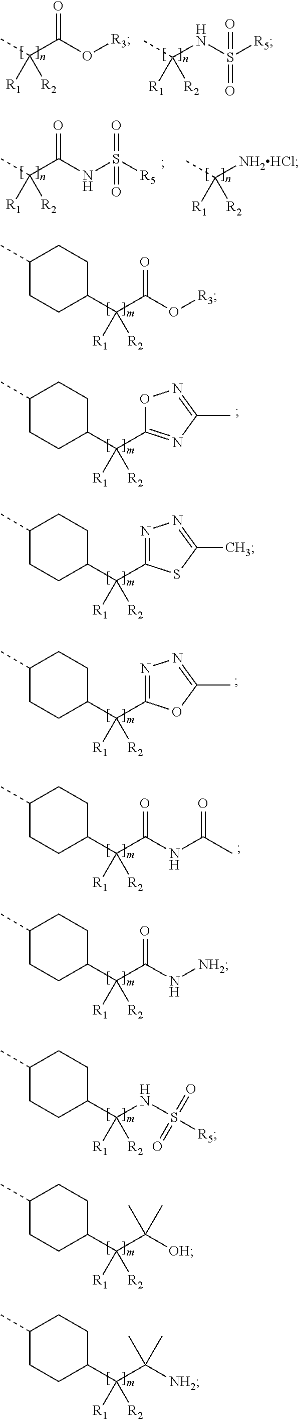

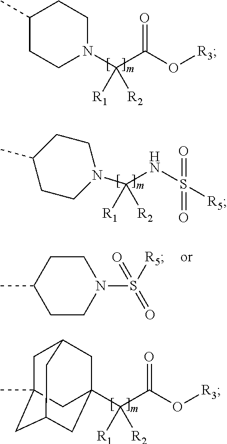

[symbol]

indicates the point of attachment;
n is an integer selected from 1-5;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring;
R₃ is hydrogen or (C₁-C₁₂)-alkyl;
R₅ is selected from hydrogen, (C₁-C₁₂)-alkyl, CF₃, (C₃-C₇)-cycloalkyl, aryl, or heterocyclyl; and
A is selected from (C₁-C₁₂)-alkyl, (C₃-C₁₂)-cycloalkyl, aryl or heterocyclyl;
wherein,
(C₁-C₁₂)-alkyl is unsubstituted or substituted with one or more groups selected from halogen, hydroxy, (C₁-C₁₂)-alkoxy, cyano, (C₃-C₁₂)-cycloalkyl, aryl or heterocyclyl;
(C₃-C₁₂)-cycloalkyl is unsubstituted or substituted with one or more groups selected from halogen, hydroxy, (C₁-C₁₂)-alkoxy, cyano, aryl or heterocyclyl;
aryl is unsubstituted or substituted with one or more groups selected from halogen, hydroxy, (C₁-C₁₂)-alkoxy, cyano, (C₁-C₁₂)-alkyl, OCF₃, CF₃, (C₃-C₁₂)-cycloalkyl, aryl, aryloxy, heterocyclyl or O-heterocyclyl; or aryl may be fused with an unsubstituted or substituted 5 or 6-membered cycloalkyl ring optionally containing one or more heteroatoms selected from O, N or S;
heterocyclyl is unsubstituted or substituted with one or more groups selected from halogen, hydroxy, (C₁-C₁₂)-alkoxy, cyano, (C₁-C₁₂)-alkyl, (C₃-C₁₂)-cycloalkyl, aryl, aryloxy, heterocyclyl or O-heterocyclyl;
with the proviso that A is not a methyl group.

In a fourteenth aspect, the present invention provides compounds of formula 1 represented by compounds of formula 1c; wherein,

### B is

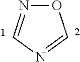

wherein 1 and 2 are the points of attachment of B to phenyl and to Z respectively;
Z is selected from:

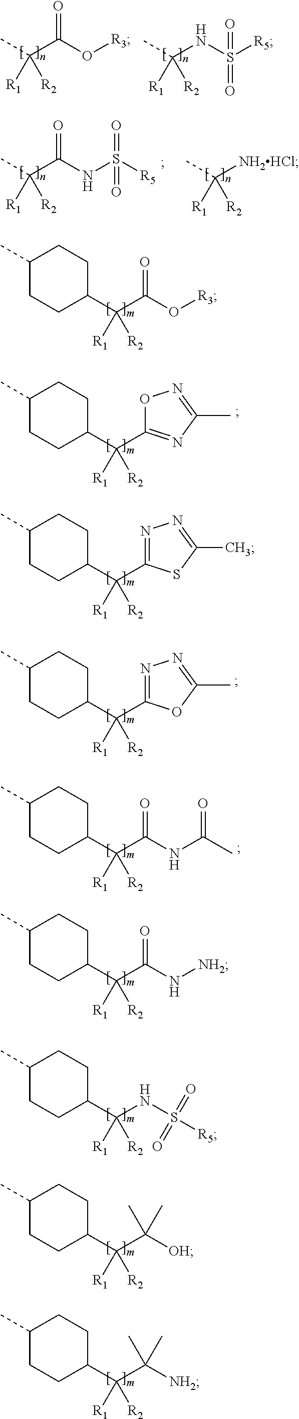

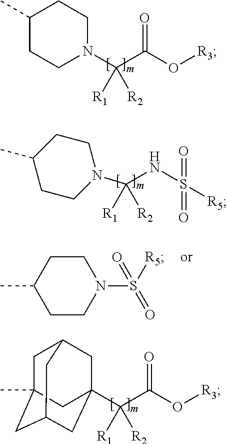

[symbol]

indicates the point of attachment;
n is an integer selected from 1-5;
m is 0 or 1:
R₁ and R₂ are independently selected from hydrogen or (C₁-C₁₂)-alkyl, or R₁ and R₂ can optionally form an unsubstituted or substituted (C₃-C₇) cycloalkyl ring;
R₃ is hydrogen or (C₁-C₁₂)-alkyl;
R₅ is selected from hydrogen, (C₁-C₁₂)-alkyl, CF₃, (C₃-C₇)-cycloalkyl, aryl, and heterocyclyl; and
A is selected from (C₁-C₁₂)-alkyl, (C₃-C₁₂)-cycloalkyl, aryl or heterocyclyl;
wherein,
(C₁-C₁₂)-alkyl is unsubstituted or substituted with one or more groups selected from halogen, hydroxy, (C₁-C₁₂)-alkoxy, cyano, (C₃-C₁₂)-cycloalkyl, aryl or heterocyclyl;
(C₃-C₁₂)-cycloalkyl is unsubstituted or substituted with one or more groups selected from halogen, hydroxy, (C₁-C₁₂)-alkoxy, cyano, aryl or heterocyclyl;
aryl is unsubstituted or substituted with one or more groups selected from halogen, hydroxy, (C₁-C₁₂)-alkoxy, cyano, (C₁-C₁₂)-alkyl, OCF₃, CF₃, (C₃-C₁₂)-cycloalkyl, aryl, aryloxy, heterocyclyl or O-heterocyclyl, or aryl may be fused with an unsubstituted or substituted 5 or 6-membered cycloalkyl ring optionally containing heteroatoms selected from O, N or S;
heterocyclyl is unsubstituted or substituted with one or more groups selected from halogen, hydroxy, (C₁-C₁₂)-alkoxy, cyano, (C₁-C₁₂)-alkyl, (C₃-C₁₂)-cycloalkyl, aryl, aryloxy, heterocyclyl or O-heterocyclyl;
with the proviso that A is not a methyl group.

In a fifteenth aspect, the present invention provides compounds of formula 1c;

wherein,

### B is

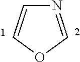

wherein 1 and 2 are the points of attachment of B to phenyl and to Z respectively;
Z is selected from:

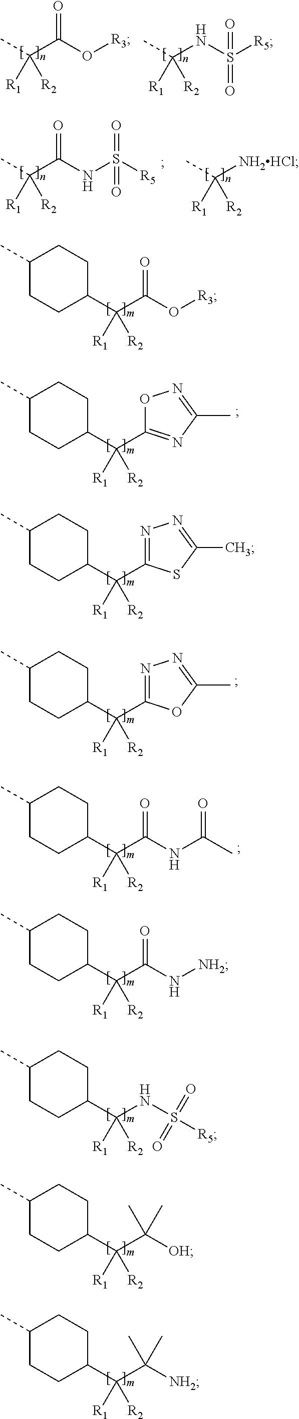

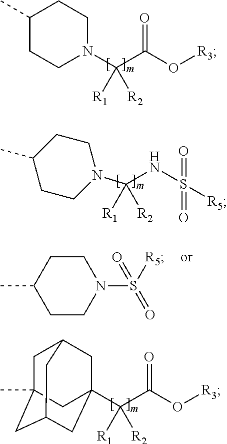

[symbol]

indicates the point of attachment;
n is an integer selected from 1-5;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring;
R₃ is hydrogen or (C₁-C₁₂)-alkyl;
R₅ is selected from hydrogen, (C₁-C₁₂)-alkyl, CF₃, (C₃-C₇)-cycloalkyl, aryl, and heterocyclyl; and
A is selected from (C₁-C₁₂)-alkyl, (C₃-C₁₂)-cycloalkyl, aryl or heterocyclyl;
wherein,
(C₁-C₁₂)-alkyl is unsubstituted or substituted with one or more groups selected from halogen, hydroxy, (C₁-C₁₂)-alkoxy, cyano, (C₃-C₁₂)-cycloalkyl, aryl or heterocyclyl;
(C₃-C₁₂)-cycloalkyl is unsubstituted or substituted with one or more groups selected from halogen, hydroxy, (C₁-C₁₂)-alkoxy, cyano, aryl or heterocyclyl;
aryl is unsubstituted or substituted with one or more groups selected from halogen, hydroxy, (C₁-C₁₂)-alkoxy, cyano, (C₁-C₁₂)-alkyl, OCF₃, CF₃, (C₃-C₁₂)-cycloalkyl, aryl, aryloxy, heterocyclyl or O-heterocyclyl, or aryl may be fused with an unsubstituted or substituted 5 or 6-membered cycloalkyl ring optionally containing heteroatoms selected from O, N or S;
heterocyclyl is unsubstituted or substituted with one or more groups selected from halogen, hydroxy, (C₁-C₁₂)-alkoxy, cyano, (C₁-C₁₂)-alkyl, (C₃-C₁₂)-cycloalkyl, aryl, aryloxy, heterocyclyl or O-heterocyclyl;
with the proviso that A is not a methyl group.

In a sixteenth aspect, the present invention provides compounds of formula 1 represented by compounds of formula 1d,

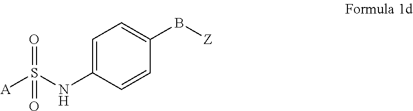

in all their stereoisomeric and tautomeric forms; and their pharmaceutically acceptable salts, solvates, polymorphs, prodrugs, carboxylic acid isosteres and N-oxides;
wherein,
Z is selected from:

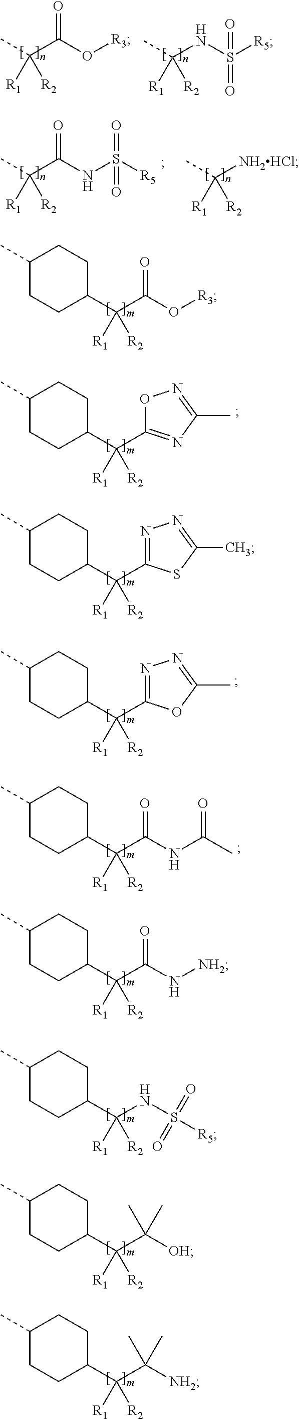

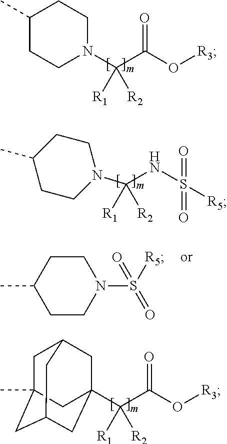

[symbol]

indicates the point of attachment;
n is an integer selected from 1-5;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring;
R₃ is hydrogen or (C₁-C₁₂)-alkyl;
R₅ is selected from hydrogen, (C₁-C₁₂)-alkyl, CF₃, (C₃-C₇)-cycloalkyl, aryl, and heterocyclyl;
B is a 5-membered heteroaryl ring represented by any one of the general structures (i) to (x);

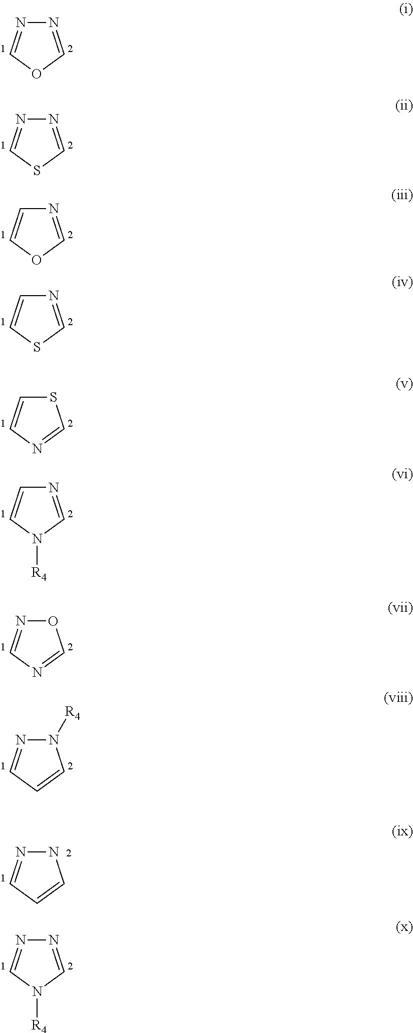

wherein 1 and 2 are the points of attachment of B to phenyl and to Z respectively and
R₅ is selected from hydrogen, (C₁-C₁₂)-alkyl or aryl; or B is a 6-membered heteroaryl ring containing 1 or 2 N-atoms, wherein the 6-membered heteroaryl ring may be unsubstituted or substituted with one or more groups selected from halogen, hydroxy, (C₁-C₁₂)-alkoxy, cyano, nitro, (C₁-C₁₂)-alkyl, (C₂-C₁₂)-alkenyl, (C₂-C₁₂)-alkynyl, (C₃-C₁₂)-cycloalkyl, aryl, aryloxy, heterocyclyl and O-heterocyclyl; and
A is selected from (C₁-C₁₂)-alkyl, (C₃-C₁₂)-cycloalkyl, aryl or heterocyclyl;
wherein,
(C₁-C₁₂)-alkyl is unsubstituted or substituted with one or more groups selected from halogen, hydroxy, (C₁-C₁₂)-alkoxy, cyano, (C₃-C₁₂)-cycloalkyl, aryl or heterocyclyl; (C₃-C₁₂)-cycloalkyl is unsubstituted or substituted with one or more groups selected from halogen, hydroxy, (C₁-C₁₂)-alkoxy, cyano, aryl or heterocyclyl; aryl is unsubstituted or substituted with one or more groups selected from halogen, hydroxy, (C₁-C₁₂)-alkoxy, cyano, (C₁-C₁₂)-alkyl, OCF₃, CF₃, (C₃-C₁₂)-cycloalkyl, aryl, aryloxy, heterocyclyl or O-heterocyclyl, or aryl may be fused with an unsubstituted or substituted 5 or 6-membered cycloalkyl ring optionally containing heteroatoms selected from O, N or S;
heterocyclyl is unsubstituted or substituted with one or more groups selected from halogen, hydroxy, (C₁-C₁₂)-alkoxy, cyano, (C₁-C₁₂)-alkyl, (C₃-C₁₂)-cycloalkyl, aryl, aryloxy, heterocyclyl or O-heterocyclyl;
with the proviso that A is not a methyl group.

In a seventeenth aspect, the present invention provides compounds of formula 1 represented by compounds of formula 1e,

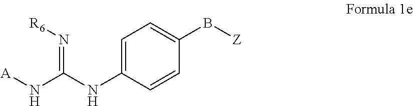

in all their stereoisomeric and tautomeric forms; and their pharmaceutically acceptable salts, solvates, polymorphs, prodrugs, carboxylic acid isosteres and N-oxides;
wherein,
Z is selected from:

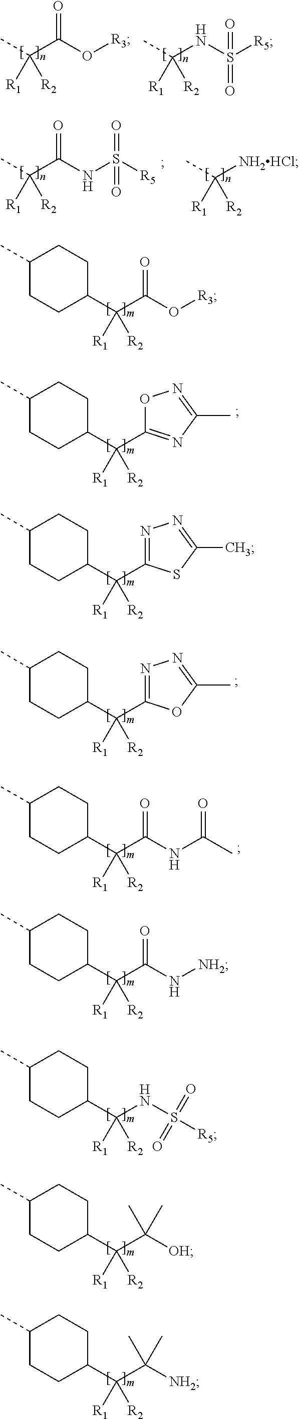

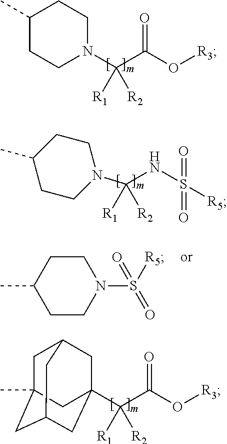

[symbol]

indicates the point of attachment;
n is an integer selected from 1-5;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring;
R₃ is hydrogen or (C₁-C₁₂)-alkyl;
R₅ is selected from hydrogen, (C₁-C₁₂)-alkyl, CF₃, (C₃-C₇)-cycloalkyl, aryl, or heterocyclyl;
B is a 5-membered heteroaryl ring represented by any one of the general structures (i) to (x);

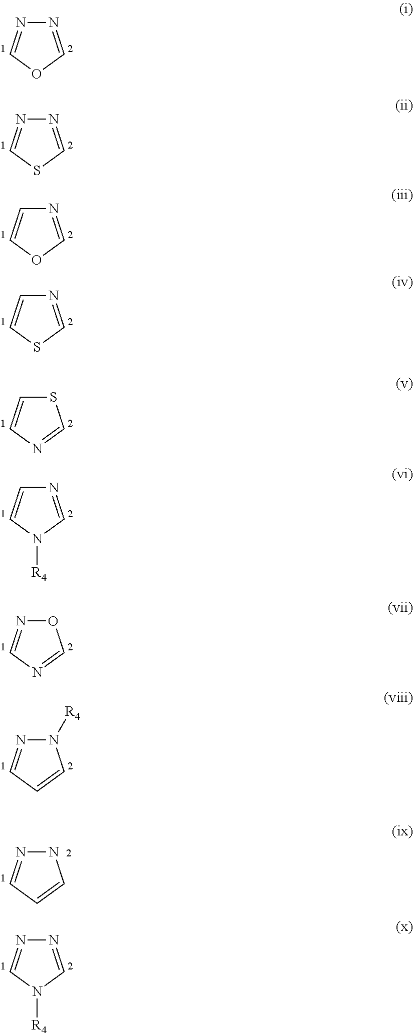

wherein 1 and 2 are the points of attachment of B to phenyl and to Z respectively and R₅ is selected from hydrogen, (C₁-C₁₂)-alkyl or aryl; or B is a 6-membered heteroaryl ring containing 1 or 2 N-atoms, wherein the 6-membered heteroaryl ring may be unsubstituted or substituted with one or more groups selected from halogen, hydroxy, (C₁-C₁₂)-alkoxy, cyano, nitro, (C₁-C₁₂)-alkyl, (C₂-C₁₂)-alkenyl, (C₂-C₁₂)-alkynyl, (C₃-C₁₂)-cycloalkyl, aryl, aryloxy, heterocyclyl or O-heterocyclyl;
R₆ is selected from hydrogen, methyl, cyano or nitro; and
A is selected from (C₁-C₁₂)-alkyl, (C₃-C₁₂)-cycloalkyl, aryl or heterocyclyl;
wherein,
(C₁-C₁₂)-alkyl is unsubstituted or substituted with one or more groups selected from halogen, hydroxy, (C₁-C₁₂)-alkoxy, cyano, (C₃-C₁₂)-cycloalkyl, aryl or heterocyclyl;
(C₃-C₁₂)-cycloalkyl is unsubstituted or substituted with one or more groups selected from halogen, hydroxy, (C₁-C₁₂)-alkoxy, cyano, aryl or heterocyclyl;
aryl is unsubstituted or substituted with one or more groups selected from halogen, hydroxy, (C₁-C₁₂)-alkoxy, cyano, (C₁-C₁₂)-alkyl, OCF₃, CF₃, (C₃-C₁₂)-cycloalkyl, aryl, aryloxy, heterocyclyl or O-heterocyclyl, or aryl may be fused with an unsubstituted or substituted 5 or 6-membered cycloalkyl ring optionally containing heteroatoms selected from O, N or S;
heterocyclyl is unsubstituted or substituted with one or more groups selected from halogen, hydroxy, (C₁-C₁₂)-alkoxy, cyano, (C₁-C₁₂)-alkyl, (C₃-C₁₂)-cycloalkyl, aryl, aryloxy, heterocyclyl or O-heterocyclyl;
with the proviso that A is not a methyl group.

In an eighteenth aspect, the present invention provides compounds of formula 1, wherein in all the above aspects and/or embodiments A is an unsubstituted aryl or an aryl substituted with one or more groups selected from halogen, hydroxy, (C₁-C₁₂)-alkoxy, cyano, unsubstituted or substituted (C₁-C₁₂)-alkyl, OCF₃, CF₃, unsubstituted or substituted (C₃-C₁₂)-cycloalkyl, unsubstituted or substituted aryl, unsubstituted or substituted aryloxy, unsubstituted or substituted heterocyclyl, or O-heterocyclyl.

In a nineteenth aspect, the present invention provides compounds of formula 1, wherein in all the above aspects and/or embodiments A is an aryl group which may be fused with an unsubstituted or substituted 5 or 6-membered cycloalkyl ring optionally containing one or more heteroatoms selected from O, N or S.

In a twentieth aspect, the present invention provides compounds of formula 1, wherein in all the above aspects and/or embodiments A is an unsubstituted heterocyclyl or a heterocyclyl substituted with one or more groups selected from halogen, hydroxy, (C₁-C₁₂)-alkoxy, cyano, unsubstituted or substituted (C₁-C₁₂)-alkyl, unsubstituted or substituted (C₃-C₁₂)-cycloalkyl, unsubstituted or substituted aryl, unsubstituted or substituted aryloxy, heterocyclyl or O-heterocyclyl.

In a twenty first aspect, the present invention provides compounds of formula 1, wherein in all the above aspects and/or embodiments A is an unsubstituted (C₃-C₁₂)-cycloalkyl or (C₃-C₁₂)-cycloalkyl substituted with one or more groups selected from halogen, hydroxy, unsubstituted or substituted (C₁-C₁₂)-alkyl, (C₁-C₁₂)-alkoxy, cyano, nitro, unsubstituted or substituted aryl, or unsubstituted or substituted heterocyclyl.

In a twenty second aspect, the present invention provides compounds of formula 1, wherein in all the above aspects and/or embodiments A is an unsubstituted (C₁-C₁₂)-alkyl or (C₁-C₁₂)-alkyl substituted with one or more groups selected from halogen, hydroxy, (C₁-C₁₂)-alkoxy, cyano, unsubstituted or substituted (C₃-C₁₂)-cycloalkyl, unsubstituted or substituted aryl, or unsubstituted or substituted heterocyclyl; with the proviso that A is not a methyl group.

In an aspect, the present invention provides compounds of formula 1, wherein m is 0.

In another aspect, the present invention provides compounds of formula 1, wherein m is 1.

In an aspect, the present invention provides compounds of formula 1, wherein n is 1.

In another aspect, the present invention provides compounds of formula 1, wherein n is 2.

In yet another aspect, the present invention provides compounds of formula 1, wherein n is 3.

In a further aspect, the present invention provides compounds of formula 1, wherein n is 4.

In a still further aspect, the present invention provides compounds of formula 1, wherein n is 5.

In an aspect, the present invention provides compounds of formula 1, wherein R₁ and R₂ are methyl groups.

In another aspect, the present invention provides compounds of formula 1, wherein R₃ is hydrogen.

In yet another aspect, the present invention provides compounds of formula 1, wherein R₃ is unsubstituted or substituted alkyl.

In another aspect, the present invention provides compounds of formula D:

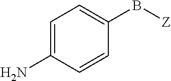

wherein B and Z are as defined in formula 1 of the first aspect of the invention; for use as intermediates in the preparation of the compounds of formula 1.

In one aspect, the present invention provides a process for the preparation of the compound of formula 1 represented by the compound of formula 1a:

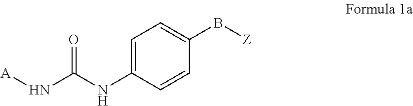

wherein A, B and Z are as defined in formula 1;
the steps comprising:
Step a) treating the compound of formula D:

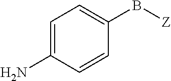

wherein B and Z are as defined in formula 1 of any one of the aspects of the invention; with a compound of formula 8 (i):

A-N═C═O 8 (i)

wherein A is as defined in formula 1 of any one of the aspects of the invention;
in a solvent selected from THF or dichloromethane at room temperature for 2-16 h;
or alternately, treating the compound of formula D:

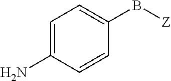

with the compound of formula 8 (ii):

A-NH₂ 8 (ii)

wherein A is as defined in formula 1 of any one of the aspects of the invention;
in presence of a coupling agent, carbonyl diimidazole in a suitable solvent such as THF at room temperature for about 24 h; and
Step b) hydrolysis of compounds of formula 1a;
wherein Z is:

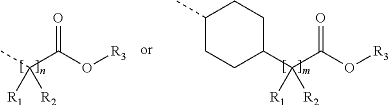

R₃ is (C₁-C₁₂)-alkyl;
by reaction with a suitable reagent such as aqueous LiOH in a suitable solvent such as THF or methanol or a mixture thereof, at room temperature for 2-16 h into the corresponding carboxylic acids of formula 1a (R₃ is H); and conversion of the carboxylic acids obtained into their corresponding pharmaceutically acceptable salts or optionally into their corresponding ester prodrugs.

The compound 8(i) used in step (a) of the above process is a commercially available compound (e.g. phenyl isocyanate).

In another aspect, the present invention provides a process for the preparation of the compound of formula 1 represented by the compound of formula 1b:

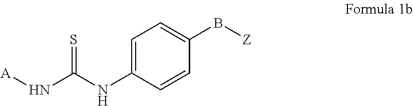

wherein A, B and Z are as defined in formula 1 of any one of the aspects of the invention;
the steps comprising:
Step a) treating the compound of formula D:

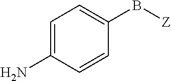

wherein B and Z are as defined in formula 1;with compound of formula 8 (iii):

A-N═C═S 8 (iii)

wherein A is as defined in formula 1 of any one of the aspects of the invention; in a suitable solvent such as THF or dichloromethane at room temperature for 2-16 h; and
Step b) hydrolysis of compounds of formula 1b;
wherein Z is:

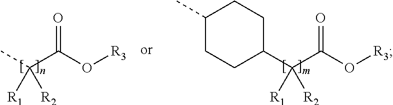

R₃ is (C₁-C₁₂)-alkyl;
by reaction with a suitable reagent such as aqueous LiOH in a suitable solvent such as THF or methanol or a mixture thereof, at room temperature for 2-16 h into the corresponding carboxylic acids of formula 1b (R₃ is H); and conversion of the carboxylic acids obtained into their corresponding pharmaceutically acceptable salts or optionally into their corresponding ester prodrugs.

In a further aspect, the present invention provides a process for the preparation of the compound of formula 1 represented by the compound of formula 1c:

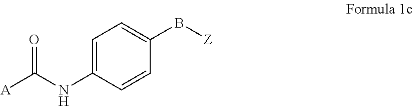

wherein A, B and Z are as defined in formula 1 of any one of the aspects of the invention;
the steps comprising:
Step a) treating the compound of formula D:

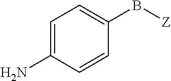

wherein B and Z are as defined in formula 1;with commercially available compound of formula 8 (iv):

A-C(O)—Cl 8 (iv)

wherein A is as defined in formula 1 of any one of the aspects of the invention;
in a suitable solvent such as dichloromethane or chloroform in a suitable base such as pyridine at room temperature for 1-2 h;
or alternately, by reacting compound of formula D:

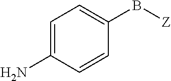

with commercially available compound of formula 8 (v):

A-COOR₃ 8(v)

wherein A and R₃ are as defined in formula 1 of any one of the aspects of the invention;
in a suitable solvent such as toluene and a coupling agent such as trimethylaluminium; and
Step b) hydrolysis of compounds of formula 1c;
wherein Z is:

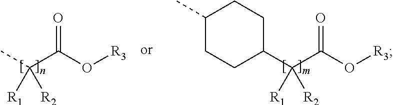

R₃ is (C₁-C₁₂)-alkyl;
by reaction with a suitable reagent such as aqueous LiOH in a suitable solvent such as THF or methanol or a mixture thereof, at room temperature for 2-16 h into the corresponding carboxylic acids of formula 1c (R₃ is H); and conversion of the carboxylic acids obtained into their corresponding pharmaceutically acceptable salts or optionally into their corresponding ester prodrugs.

In a still further aspect, the present invention provides a process for the preparation of the compound of formula 1 represented by the compound of formula 1d:

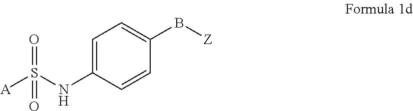

wherein A, B and Z are as defined in formula 1 of any one of the aspects of the invention;
the steps comprising:
Step a) treating the compound of formula D:

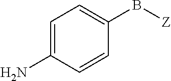

wherein B and Z are as defined in formula 1 of any one of the aspects of the invention; with compound of formula 8 (vi):

A-SO₂—Cl 8 (vi)

wherein A is as defined in formula 1;
in a suitable solvent such as dichloromethane or chloroform in a suitable base such as pyridine at room temperature for 1-2 h; and
Step b) hydrolysis of compounds of formula 1d;
wherein Z is:

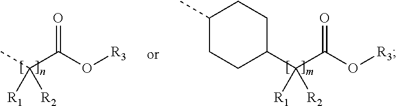

R₃ is (C₁-C₁₂)-alkyl;
by reaction with a suitable reagent such as aqueous LiOH in a suitable solvent such as THF or methanol or a mixture thereof, at room temperature for 2-16 h into the corresponding carboxylic acids of formula 1d (R₃ is H); and conversion of the carboxylic acids obtained into their corresponding pharmaceutically acceptable salts or optionally into their corresponding ester prodrugs.

In a still further aspect, the present invention provides a process for the preparation of the compound of formula 1 represented by the compound of formula 1e:

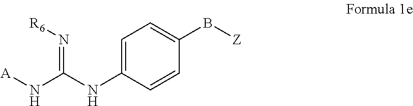

wherein A, B, Z and R₆ are as defined in formula 1 of any one of the aspects of the invention;
the steps comprising:
Step a) reacting the compound of formula 1b:

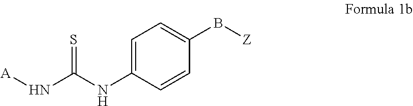

with the compound of formula 8 (vii):

R₆—NH₂ 8 (vii)

wherein R₆ is as defined in formula 1 according to any one of the aspects of the invention;
in presence of HgO in a suitable solvent such as methanol at room temperature for 1-3 h; and
Step b) hydrolysis of compounds of formula 1e;
wherein Z is:

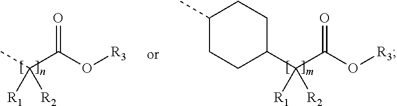

R₃ is (C₁-C₁₂)-alkyl;
by reaction with a suitable reagent such as aqueous LiOH in a suitable solvent such as THF or methanol or a mixture thereof, at room temperature for 2-16 h into the corresponding carboxylic acids of formula 1e (R₃ is H); and conversion of the carboxylic acids obtained into their corresponding pharmaceutically acceptable salts or optionally into their corresponding ester prodrugs.

In an aspect, the present invention provides compounds of formula 1 selected from:
Methyl 3-(5-(4-(3-(3-(trifluoromethyl)phenyl)ureido)phenyl)thiazol-2-yl)propanoate;
3-(5-(4-(3-(3-(Trifluoromethyl)phenyl)ureido)phenyl)thiazol-2-yl)propanoic acid;
Methyl 3-(5-(4-(3-(2-chlorophenyl)ureido)phenyl)thiazol-2-yl)propanoate;
3-(5-(4-(3-(2-Chlorophenyl)ureido)phenyl)thiazol-2-yl)propanoic acid;
Methyl 3-(5-(4-(3-cyclohexylureido)phenyl)thiazol-2-yl)propanoate;
3-(5-(4-(3-Cyclohexylureido)phenyl)thiazol-2-yl)propanoic acid;
Methyl 3-(5-(4-(3-(4-chloro-2-phenoxyphenyl)ureido)phenyl)thiazol-2-yl)propanoate;
3-(5-(4-(3-(4-Chloro-2-phenoxyphenyl)ureido)phenyl)thiazol-2-yl)propanoic acid;
Methyl 3-(5-(4-(4-t-butylbenzamido)phenyl)thiazol-2-yl)propanoate;
3-(5-(4-(4-t-butylbenzamido)phenyl)thiazol-2-yl)propanoic acid;
Methyl 3-(5-(4-(4-pentylbenzamido)phenyl)thiazol-2-yl)propanoate;
3-(5-(4-(4-Pentylbenzamido)phenyl)thiazol-2-yl)propanoic acid;
Methyl 3-(5-(4-(3-ethoxy-5-(methoxymethyl)benzamido)phenyl)thiazol-2-yl)propanoate;
3-(5-(4-(3-Ethoxy-5-(methoxymethyl)benzamido)phenyl)thiazol-2-yl)propanoic acid;
Methyl 3-(5-(4-(4-pentylbenzamido)phenyl)thiazol-2-yl)propanoate;
3-(5-(4-(2-Naphthamido)phenyl)thiazol-2-yl)propanoic acid;
Methyl 3-(5-(4-(4-butoxybenzamido)phenyl)thiazol-2-yl)propanoate;
3-(5-(4-(4-Butoxybenzamido)phenyl)thiazol-2-yl)propanoic acid;
Methyl 3-(5-(4-(2,4-dimethoxyphenylsulfonamido)phenyl)thiazol-2-yl)propanoate;
3-(5-(4-(2,4-Dimethoxyphenylsulfonamido)phenyl)thiazol-2-yl)propanoic acid;
Methyl 3-(5-(4-(3-(2-chlorophenyl)ureido)phenyl)thiazol-2-yl)-2,2-dimethyl propanoate;
3-(5-(4-(3-(2-Chlorophenyl)ureido)phenyl)thiazol-2-yl)-2,2-dimethylpropanoic acid;
Methyl 2,2-dimethyl-3-(5-(4-(3-(4-(trifluoromethyl)phenyl)ureido)phenyl)thiazol-2-yl)propanoate;
2,2-Dimethyl-3-(5-(4-(3-(4-(trifluoromethyl)phenyl)ureido)phenyl)thiazol-2-yl)propanoic acid;
Methyl 2,2-dimethyl-3-(5-(4-(3-(4-(trifluoromethyl)phenyl)ureido)phenyl)thiazol-2-yl)propanoate;
3-(5-(4-(3-(4-Fluorophenyl)ureido)phenyl)thiazol-2-yl)-2,2-dimethylpropanoic acid;
Methyl 3-(5-(4-(3-(4-methoxyphenyl)ureido)phenyl)thiazol-2-yl)-2,2-dimethyl propanoate;
3-(5-(4-(3-(4-Methoxyphenyl)ureido)phenyl)thiazol-2-yl)-2,2-dimethyl propanoic acid;
Methyl 3-(5-(4-(3-cyclohexylureido)phenyl)thiazol-2-yl)-2,2-dimethyl propanoate;
3-(5-(4-(3-Cyclohexylureido)phenyl)thiazol-2-yl)-2,2-dimethylpropanoic acid;
Methyl 3-(5-(4-(3-(4-chloro-2-phenoxyphenyl)ureido)phenyl)thiazol-2-yl)-2,2-dimethylpropanoate;
3-(5-(4-(3-(4-Chloro-2-phenoxyphenyl)ureido)phenyl)thiazol-2-yl)-2,2-dimethylpropanoic acid;
Methyl 3-(5-(4-(4-tert-butylbenzamido)phenyl)thiazol-2-yl)-2,2-dimethyl propanoate;
3-(5-(4-(4-t-Butylbenzamido)phenyl)thiazol-2-yl)-2,2-dimethylpropanoic acid;
Methyl 3-(5-(4-biphenyl-4-ylcarboxamidophenyl)thiazol-2-yl)-2,2-dimethyl propanoate;
3-(5-(4-Biphenyl-4-ylcarboxamidophenyl)thiazol-2-yl)-2,2-dimethylpropanoic acid;
Methyl 4-(5-(4-(3-(3-(trifluoromethyl)phenyl)ureido)phenyl)thiazol-2-yl)butanoate;
4-(5-(4-(3-(3-(Trifluoromethyl)phenyl)ureido)phenyl)thiazol-2-yl)butanoic acid;
Methyl 4-(5-(4-(3-(2-chlorophenyl)ureido)phenyl)thiazol-2-yl)butanoate;
4-(5-(4-(3-(2-Chlorophenyl)ureido)phenyl)thiazol-2-yl)butanoic acid;
Methyl 4-(5-(4-(3-(3,4-dimethylphenyl)ureido)phenyl)thiazol-2-yl)butanoate;
4-(5-(4-(3-(3,4-Dimethylphenyl)ureido)phenyl)thiazol-2-yl)butanoic acid;
Methyl 4-(5-(4-(3-(4-chloro-2-phenoxyphenyl)ureido)phenyl)thiazol-2-yl)butanoate;
4-(5-(4-(3-(4-Chloro-2-phenoxyphenyl)ureido)phenyl)thiazol-2-yl)butanoic acid;
Methyl 4-(5-(4-(4-t-butylbenzamido)phenyl)thiazol-2-yl)butanoate;
4-(5-(4-(4-t-Butylbenzamido)phenyl)thiazol-2-yl)butanoic acid;
Methyl 4-(5-(4-(4-pentylbenzamido)phenyl)thiazol-2-yl)butanoate;
4-(5-(4-(4-Pentylbenzamido)phenyl)thiazol-2-yl)butanoic acid;
Methyl 4-(5-(4-biphenyl-4-ylcarboxamidophenyl)thiazol-2-yl)butanoate;
4-(5-(4-Biphenyl-4-ylcarboxamidophenyl)thiazol-2-yl)butanoic acid;
Methyl 4-(5-(4-(2,4-dimethoxyphenylsulfonamido)phenyl)thiazol-2-yl)butanoate;
4-(5-(4-(2,4-Dimethoxyphenylsulfonamido)phenyl)thiazol-2-yl)butanoic acid;
Methyl 3,3-dimethyl-4-(5-(4-(3-(3-(trifluoromethyl)phenyl)ureido)phenyl)thiazol-2-yl)butanoate;
3,3-Dimethyl-4-(5-(4-(3-(3-(trifluoromethyl)phenyl)ureido)phenyl)thiazol-2-yl)butanoic acid;
Methyl 4-(5-(4-(3-(2-chlorophenyl)ureido)phenyl)thiazol-2-yl)-3,3-dimethyl butanoate;
4-(5-(4-(3-(2-Chlorophenyl)ureido)phenyl)thiazol-2-yl)-3,3-dimethylbutanoic acid;
Methyl 4-(5-(4-(3-(4-chloro-2-phenoxyphenyl)ureido)phenyl)thiazol-2-yl)-3,3-dimethylbutanoate;
4-(5-(4-(3-(4-Chloro-2-phenoxyphenyl)ureido)phenyl)thiazol-2-yl)-3,3-dimethyl butanoic acid;
Methyl 4-(5-(4-(4-tert-butylbenzamido)phenyl)thiazol-2-yl)-3,3-dimethyl butanoate;
4-(5-(4-(4-t-Butylbenzamido)phenyl)thiazol-2-yl)-3,3-dimethylbutanoic acid;
Methyl 4-(5-(4-biphenyl-4-ylcarboxamidophenyl)thiazol-2-yl)-3,3-dimethyl butanoate;
4-(5-(4-Biphenyl-4-ylcarboxamidophenyl)thiazol-2-yl)-3,3-dimethylbutanoic acid;
Methyl 3,3-dimethyl-4-(5-(4-(4-pentylbenzamido)phenyl)thiazol-2-yl)butanoate;
3,3-Dimethyl-4-(5-(4-(4-pentylbenzamido)phenyl)thiazol-2-yl)butanoic acid;
Methyl 4-(5-(4-(2,4-dimethoxyphenylsulfonamido)phenyl)thiazol-2-yl)-3,3-dimethylbutanoate;
4-(5-(4-(2,4-Dimethoxyphenylsulfonamido)phenyl)thiazol-2-yl)-3,3-dimethylbutanoic acid;
Methyl 2,2-dimethyl-4-(5-(4-(3-(3-(trifluoromethyl)phenyl)ureido)phenyl)thiazol-2-yl)butanoate;
2,2-Dimethyl-4-(5-(4-(3-(3-(trifluoromethyl)phenyl)ureido)phenyl)thiazol-2-yl)butanoic acid;
Methyl 4-(5-(4-(3-(2-chlorophenyl)ureido)phenyl)thiazol-2-yl)-2,2-dimethyl butanoate;
4-(5-(4-(3-(2-Chlorophenyl)ureido)phenyl)thiazol-2-yl)-2,2-dimethylbutanoic acid;
Methyl 4-(5-(4-(3-(4-chloro-2-phenoxyphenyl)ureido)phenyl)thiazol-2-yl)-2,2-dimethylbutanoate;
4-(5-(4-(3-(4-Chloro-2-phenoxyphenyl)ureido)phenyl)thiazol-2-yl)-2,2-dimethylbutanoic acid;
Methyl 4-(5-(4-(3-cyclohexylureido)phenyl)thiazol-2-yl)-2,2-dimethyl butanoate;
4-(5-(4-(3-Cyclohexylureido)phenyl)thiazol-2-yl)-2,2-dimethylbutanoic acid;
Methyl 4-(5-(4-(3-(4-fluorophenyl)ureido)phenyl)thiazol-2-yl)y-2,2-dimethyl butanoate;
4-(5-(4-(3-(2-Chlorophenyl)ureido)phenyl)thiazol-2-yl)-2,2-dimethylbutanoic acid;
Methyl 4-(5-(4-(3-(4-methoxyphenyl)ureido)phenyl)thiazol-2-yl)-2,2-dimethyl butanoate;
4-(5-(4-(3-(4-Methoxyphenyl)ureido)phenyl)thiazol-2-yl)-2,2-dimethyl butanoic acid;
Methyl 4-(5-(4-(3-(4-isopropylphenyl)ureido)phenyl)thiazol-2-yl)-2,2-dimethyl butanoate;
4-(5-(4-(3-(4-Isopropylphenyl)ureido)phenyl)thiazol-2-yl)-2,2-dimethyl butanoic acid;
Methyl 4-(5-(4-(3-(2,4-difluorophenyl)ureido)phenyl)thiazol-2-yl)-2,2-dimethylbutanoate;
4-(5-(4-(3-(2,4-Difluorophenyl)ureido)phenyl)thiazol-2-yl)-2,2-dimethyl butanoic acid;
Methyl 4-(5-(4-(3-(2-fluorophenyl)ureido)phenyl)thiazol-2-yl)-2,2-dimethyl butanoate;
4-(4-(4-(3-(2-Fluorophenyl)ureido)phenyl)-3H-pyrrol-2-yl)-2,2-dimethyl butanoic acid;
Methyl 4-(5-(4-(4-t-butylbenzamido)phenyl)thiazol-2-yl)-2,2-dimethyl butanoate;
4-(5-(4-(4-t-butylbenzamido)phenyl)thiazol-2-yl)-2,2-dimethylbutanoic acid;
Methyl 4-(5-(4-biphenyl-4-ylcarboxamidophenyl)thiazol-2-yl)-2,2-dimethyl butanoate;
4-(5-(4-Biphenyl-4-ylcarboxamidophenyl)thiazol-2-yl)-2,2-dimethylbutanoic acid;
Methyl 2,2-dimethyl-4-(5-(4-(4-(oxazol-5-yl)benzamido)phenyl)thiazol-2-yl)butanoate;
2,2-Dimethyl-4-(5-(4-(4-(oxazol-5-yl)benzamido)phenyl)thiazol-2-yl)butanoic acid;
Methyl 2,2-dimethyl-4-(5-(4-(4-phenylthiazole-2-carboxamido)phenyl)thiazol-2-yl)butanoate;
2,2-Dimethyl-4-(5-(4-(4-phenylthiazole-2-carboxamido)phenyl)thiazol-2-yl)butanoic acid;
Methyl 3-(5-(4-(3-(2-chlorophenyl)ureido)phenyl)oxazol-2-yl)-2,2-dimethyl propanoate;
3-(5-(4-(3-(2-Chlorophenyl)ureido)phenyl)oxazol-2-yl)-2,2-dimethylpropanoic acid;
Methyl 2,2-dimethyl-3-(5-(4-(3-(4-(trifluoromethyl)phenyl)ureido)phenyl)oxazol-2-yl)propanoate;
2,2-Dimethyl-3-(5-(4-(3-(4-(trifluoromethyl)phenyl)ureido)phenyl)oxazol-2-yl)propanoic acid;
Methyl 3-(5-(4-(3-(4-fluorophenyl)ureido)phenyl)oxazol-2-yl)-2,2-dimethyl propanoate;
3-(5-(4-(3-(4-Fluorophenyl)ureido)phenyl)oxazol-2-yl)-2,2-dimethylpropanoic acid;
Methyl 3-(5-(4-(3-(4-methoxyphenyl)ureido)phenyl)oxazol-2-yl)-2,2-dimethyl propanoate;
3-(5-(4-(3-(4-Methoxyphenyl)ureido)phenyl)oxazol-2-yl)-2,2-dimethyl propanoic acid;
Methyl 3-(5-(4-(3-(4-chloro-2-phenoxyphenyl)ureido)phenyl)oxazol-2-yl)-2,2-dimethylpropanoate;
3-(5-(4-(3-(4-Chloro-2-phenoxyphenyl)ureido)phenyl)oxazol-2-yl)-2,2-dimethyl propanoic acid;
Methyl 3-(5-(4-(4-t-butylbenzamido)phenyl)oxazol-2-yl)-2,2-dimethyl propanoate;
3-(5-(4-(4-t-Butylbenzamido)phenyl)oxazol-2-yl)-2,2-dimethylpropanoic acid;
Methyl 3-(5-(4-biphenyl-4-ylcarboxamidophenyl)oxazol-2-yl)-2,2-dimethyl propanoate;
3-(5-(4-Biphenyl-4-ylcarboxamidophenyl)oxazol-2-yl)-2,2-dimethylpropanoic acid;
Methyl 4-(5-(4-(3-(3-(trifluoromethyl)phenyl)ureido)phenyl)thiazol-2-yl)cyclohexanecarboxylate;
4-(5-(4-(3-(3-(Trifluoromethyl)phenyl)ureido)phenyl)thiazol-2-yl)cyclohexane carboxylic acid;
Methyl 4-(5-(4-(3-p-tolylureido)phenyl)thiazol-2-yl)cyclohexanecarboxylate;
4-(5-(4-(3-p-Tolylureido)phenyl)thiazol-2-yl)cyclohexanecarboxylic acid;
Methyl 4-(5-(4-(3-(2,4-difluorophenyl)ureido)phenyl)thiazol-2-yl)cyclohexane carboxylate;
4-(5-(4-(3-(2,4-Difluorophenyl)ureido)phenyl)thiazol-2-yl)cyclohexane carboxylic acid;
Methyl 4-(5-(4-(3-(2-fluorophenyl)ureido)phenyl)thiazol-2-yl)cyclohexane carboxylate;
4-(5-(4-(3-(2-Fluorophenyl)ureido)phenyl)thiazol-2-yl)cyclohexanecarboxylic acid;
Methyl 4-(5-(4-(3-cyclohexylureido)phenyl)thiazol-2-yl)cyclohexane carboxylate;
4-(5-(4-(3-Cyclohexylureido)phenyl)thiazol-2-yl)cyclohexanecarboxylic acid;
Methyl 4-(5-(4-(3-(3-chlorophenyl)ureido)phenyl)thiazol-2-yl)cyclohexane carboxylate;
4-(5-(4-(3-(3-Chlorophenyl)ureido)phenyl)thiazol-2-yl)cyclohexanecarboxylic acid;
Methyl 4-(5-(4-(3-(4-chlorophenyl)ureido)phenyl)thiazol-2-yl)cyclohexane carboxylate;
4-(5-(4-(3-(4-Chlorophenyl)ureido)phenyl)thiazol-2-yl)cyclohexanecarboxylic acid;
Methyl 4-(5-(4-(3-(2-chloro-4-(trifluoromethyl)phenyl)ureido)phenyl)thiazol-2-yl)cyclohexanecarboxylate;
4-(5-(4-(3-(2-Chloro-4-(trifluoromethyl)phenyl)ureido)phenyl)thiazol-2-yl)cyclohexanecarboxylic acid;
Methyl 4-(5-(4-(3-(2-chloro-5-methylphenyl)ureido)phenyl)thiazol-2-yl)cyclo hexanecarboxylate;
4-(5-(4-(3-(2-Chloro-5-methylphenyl)ureido)phenyl)thiazol-2-yl)cyclohexane carboxylic acid;
Methyl 4-(5-(4-(3-(3-chloro-2-fluorophenyl)ureido)phenyl)thiazol-2-yl)cyclo hexanecarboxylate;
4-(5-(4-(3-(3-Chloro-2-fluorophenyl)ureido)phenyl)thiazol-2-yl)cyclohexane carboxylic acid;
Methyl 4-(5-(4-(3-(4-methoxy-2-methylphenyl)ureido)phenyl)thiazol-2-yl)cyclohexanecarboxylate;
4-(5-(4-(3-(4-Methoxy-2-methylphenyl)ureido)phenyl)thiazol-2-yl)cyclo hexanecarboxylic acid;
Methyl 4-(5-(4-(3-benzo[d][1,3]dioxol-5-ylureido)phenyl)thiazol-2-yl)cyclohexanecarboxylate;
4-(5-(4-(3-Benzo[d][1,3]dioxol-5-ylureido)phenyl)thiazol-2-yl)cyclohexane carboxylic acid;
Methyl 4-(5-(4-(3-(2-chloro-6-(trifluoromethyl)phenyl)ureido)phenyl)thiazol-2-yl)cyclohexanecarboxylate;
4-(5-(4-(3-(2-Chloro-6-(trifluoromethyl)phenyl)ureido)phenyl)thiazol-2-yl)cyclohexanecarboxylic acid;
Methyl 4-(5-(4-(3-(4-chloro-2-(trifluoromethyl)phenyl)ureido)phenyl)thiazol-2-yl)cyclohexanecarboxylate;
4-(5-(4-(3-(4-Chloro-2-(trifluoromethyl)phenyl)ureido)phenyl)thiazol-2-yl)cyclohexanecarboxylic acid;
Methyl 4-(5-(4-(3-(2-chloro-6-methylphenyl)ureido)phenyl)thiazol-2-yl)cyclo hexanecarboxylate;
4-(5-(4-(3-(2-Chloro-6-methylphenyl)ureido)phenyl)thiazol-2-yl)cyclohexane carboxylic acid;
Methyl 4-(5-(4-(3-(5-chloro-2-methylphenyl)ureido)phenyl)thiazol-2-yl)cyclo hexanecarboxylate;
4-(5-(4-(3-(5-Chloro-2-methylphenyl)ureido)phenyl)thiazol-2-yl)cyclohexane carboxylic acid;
Methyl 4-(5-(4-(3-(2-(trifluoromethyl)phenyl)ureido)phenyl)thiazol-2-yl)cyclo hexanecarboxylate;
4-(5-(4-(3-(2-(Trifluoromethyl)phenyl)ureido)phenyl)thiazol-2-yl)cyclohexane carboxylic acid;
Methyl 4-(5-(4-(3-(2-(trifluoromethoxy)phenyl)ureido)phenyl)thiazol-2-yl)cyclohexanecarboxylate;
4-(5-(4-(3-(2-(Trifluoromethoxy)phenyl)ureido)phenyl)thiazol-2-yl)cyclohexane carboxylic acid;
Methyl 4-(5-(4-(3-(4-phenoxyphenyl)ureido)phenyl)thiazol-2-yl)cyclohexane carboxylate;
4-(5-(4-(3-(4-Phenoxyphenyl)ureido)phenyl)thiazol-2-yl)cyclohexane carboxylic acid;
Methyl 4-(5-(4-(3-(4-chloro-2-fluorophenyl)ureido)phenyl)thiazol-2-yl)cyclo hexanecarboxylate;
4-(5-(4-(3-(4-Chloro-2-fluorophenyl)ureido)phenyl)thiazol-2-yl)cyclohexane carboxylic acid;
Methyl 4-(5-(4-(3-(2-fluoro-5-methylphenyl)ureido)phenyl)thiazol-2-yl)cyclo hexanecarboxylate;
4-(5-(4-(3-(2-Fluoro-5-methylphenyl)ureido)phenyl)thiazol-2-yl)cyclohexane carboxylic acid;
Methyl 4-(5-(4-(3-(2-fluoro-6-(trifluoromethyl)phenyl)ureido)phenyl)thiazol-2-yl)cyclohexanecarboxylate;
4-(5-(4-(3-(2-Fluoro-6-(trifluoromethyl)phenyl)ureido)phenyl)thiazol-2-yl)cyclohexanecarboxylic acid;
Methyl 4-(5-(4-(3-(3-fluorophenyl)ureido)phenyl)thiazol-2-yl)cyclohexane carboxylate;
4-(5-(4-(3-(3-Fluorophenyl)ureido)phenyl)thiazol-2-yl)cyclohexanecarboxylic acid;
Methyl 4-(5-(4-(3-(3,4-difluorophenyl)ureido)phenyl)thiazol-2-yl)cyclohexane carboxylate;
4-(5-(4-(3-(3,4-Difluorophenyl)ureido)phenyl)thiazol-2-yl)cyclohexane carboxylic acid;
Methyl 4-(5-(4-(3-(3,5-difluorophenyl)ureido)phenyl)thiazol-2-yl)cyclohexane carboxylate;
4-(5-(4-(3-(3,5-Difluorophenyl)ureido)phenyl)thiazol-2-yl)cyclohexane carboxylic acid;
Methyl 4-(5-(4-(3-(2,6-difluorophenyl)ureido)phenyl)thiazol-2-yl)cyclohexane carboxylate;
4-(5-(4-(3-(2,6-Difluorophenyl)ureido)phenyl)thiazol-2-yl)cyclohexane carboxylic acid;
Methyl 4-(5-(4-(3-(2,3,4-trifluorophenyl)ureido)phenyl)thiazol-2-yl)cyclo hexanecarboxylate;
4-(5-(4-(3-(2,3,4-Trifluorophenyl)ureido)phenyl)thiazol-2-yl)cyclohexane carboxylic acid;
Methyl 4-(5-(4-(3-(2-chlorophenyl)ureido)phenyl)thiazol-2-yl)cyclohexane carboxylate;
4-(5-(4-(3-(2-Chlorophenyl)ureido)phenyl)thiazol-2-yl)cyclohexanecarboxylic acid;
Methyl 4-(5-(4-(3-(4-chloro-2-phenoxyphenyl)ureido)phenyl)thiazol-2-yl)cyclohexanecarboxylate;
4-(5-(4-(3-(4-Chloro-2-phenoxyphenyl)ureido)phenyl)thiazol-2-yl)cyclohexane carboxylic acid;
Methyl 4-(5-(4-(3-phenylureido)phenyl)thiazol-2-yl)cyclohexanecarboxylate;
4-(5-(4-(3-Phenylureido)phenyl)thiazol-2-yl)cyclohexanecarboxylic acid;
Methyl 4-(5-(4-(4-t-butylbenzamido)phenyl)thiazol-2-yl)cyclohexane carboxylate;
4-(5-(4-(4-t-Butylbenzamido)phenyl)thiazol-2-yl)cyclohexanecarboxylic acid;
Methyl 4-(5-(4-(2-chlorobenzamido)phenyl)thiazol-2-yl)cyclohexane carboxylate;
4-(5-(4-(2-Chlorobenzamido)phenyl)thiazol-2-yl)cyclohexanecarboxylic acid;
Methyl 4-(5-(4-(5-phenyloxazole-2-carboxamido)phenyl)thiazol-2-yl)cyclohexane carboxylate;
4-(5-(4-(5-Phenyloxazole-2-carboxamido)phenyl)thiazol-2-yl)cyclohexane carboxylic acid;
Methyl 4-(5-(4-(3-(4-methoxyphenyl)thioureido)phenyl)thiazol-2-yl)cyclo hexanecarboxylate;
Methyl 4-(5-(4-(3-(4-chlorophenyl)thioureido)phenyl)thiazol-2-yl)cyclo hexanecarboxylate;
Methyl 4-(5-(4-(3-(2-chlorophenyl)ureido)phenyl)oxazol-2-yl)cyclohexane carboxylate;
4-(5-(4-(3-(2-Chlorophenyl)ureido)phenyl)oxazol-2-yl)cyclohexanecarboxylic acid;
Methyl 4-(5-(4-(3-phenylureido)phenyl)oxazol-2-yl)cyclohexanecarboxylate;
4-(5-(4-(3-Phenylureido)phenyl)oxazol-2-yl)cyclohexanecarboxylic acid;
Methyl 4-(5-(4-(3-(3-chlorophenyl)ureido)phenyl)oxazol-2-yl)cyclohexane carboxylate;
4-(5-(4-(3-(3-Chlorophenyl)ureido)phenyl)oxazol-2-yl)cyclohexanecarboxylic acid;
Methyl 4-(5-(4-(3-(2-methoxyphenyl)ureido)phenyl)oxazol-2-yl)cyclohexane carboxylate;
4-(5-(4-(3-(2-Methoxyphenyl)ureido)phenyl)oxazol-2-yl)cyclohexane carboxylic acid;
Methyl 4-(5-(4-(2-chlorobenzamido)phenyl)oxazol-2-yl)cyclohexane carboxylate;
4-(5-(4-(2-Chlorobenzamido)phenyl)oxazol-2-yl)cyclohexanecarboxylic acid;
Methyl 4-(5-(4-(4-t-butylbenzamido)phenyl)oxazol-2-yl)cyclohexane carboxylate;
4-(5-(4-(4-t-Butylbenzamido)phenyl)oxazol-2-yl)cyclohexanecarboxylic acid;
(1 r,4r)-Methyl 4-(3-(4-(3-(2-chlorophenyl)ureido)phenyl)-1,2,4-oxadiazol-5-yl)cyclohexanecarboxylate;
(1r,4r)-4-(3-(4-(3-(2-Chlorophenyl)ureido)phenyl)-1,2,4-oxadiazol-5-yl)cyclo hexanecarboxylic acid;
(1r,4r)-Methyl 4-(3-(4-(3-(2,4-difluorophenyl)ureido)phenyl)-1,2,4-oxadiazol-5-yl)cyclohexanecarboxylate;
(1r,4r)-4-(3-(4-(3-(2,4-Difluorophenyl)ureido)phenyl)-1,2,4-oxadiazol-5-yl)cyclohexanecarboxylic acid;
(1r,4r)-Methyl 4-(3-(4-(3-p-tolylureido)phenyl)-1,2,4-oxadiazol-5-yl)cyclo hexane carboxylate;
(1r,4r)-4-(3-(4-(3-p-Tolylureido)phenyl)-1,2,4-oxadiazol-5-yl)cyclohexane carboxylic acid;
(1r,4r)-Methyl 4-(3-(4-(3-(3-chlorophenyl)ureido)phenyl)-1,2,4-oxadiazol-5-yl)cyclo hexanecarboxylate;
(1r,4r)-4-(3-(4-(3-(3-Chlorophenyl)ureido)phenyl)-1,2,4-oxadiazol-5-yl)cyclo hexanecarboxylic acid;
(1r,4r)-Methyl 4-(3-(4-(3-(4-chloro-2-phenoxyphenyl)ureido)phenyl)-1,2,4-oxadiazol-5-yl)cyclohexanecarboxylate;
(1r,4r)-4-(3-(4-(3-(4-Chloro-2-phenoxyphenyl)ureido)phenyl)-1,2,4-oxadiazol-5-yl)cyclohexanecarboxylic acid;
(1r,4r)-Methyl 4-(3-(4-(4-tert-butylbenzamido)phenyl)-1,2,4-oxadiazol-5-yl)cyclohexanecarboxylate;
(1r,4r)-4-(3-(4-(4-t-Butylbenzamido)phenyl)-1,2,4-oxadiazol-5-yl)cyclohexane carboxylic acid;
(1r,4r)-Methyl 4-(3-(4-biphenyl-4-ylcarboxamidophenyl)-1,2,4-oxadiazol-5-yl)cyclohexanecarboxylate;
(1r,4r)-4-(3-(4-Biphenyl-4-ylcarboxamidophenyl)-1,2,4-oxadiazol-5-yl)cyclo hexanecarboxylic acid;
(1r,4r)-Methyl 4-(3-(4-(4-(trifluoromethoxy)benzamido)phenyl)-1,2,4-oxadiazol-5-yl)cyclohexanecarboxylate;
(1r,4r)-4-(3-(4-(4-(Trifluoromethoxy)benzamido)phenyl)-1,2,4-oxadiazol-5-yl)cyclohexanecarboxylic acid;
Methyl 4-(5-(4-(3-(3,5-difluorophenyl)ureido)phenyl)thiazol-2-yl)-2,2-dimethylbutanoate;
4-(5-(4-(3-(3,5-Difluorophenyl)ureido)phenyl)thiazol-2-yl)-2,2-dimethylbutanoic acid;
Sodium salt of 4-(5-(4-(3-(3,5-difluorophenyl)ureido)phenyl)thiazol-2-yl)-2,2-dimethylbutanoate;
Methyl 2,2-dimethyl-4-(5-(4-(3-(2,4,5-trifluorophenyl)ureido)phenyl)thiazol-2-yl)butanoate;
2,2-Dimethyl-4-(5-(4-(3-(2,4,5-trifluorophenyl)ureido)phenyl)thiazol-2-yl)butanoic acid;
Sodium salt of 2,2-dimethyl-4-(5-(4-(3-(2,4,5-trifluorophenyl)ureido)phenyl)thiazol-2-yl)butanoate;
Methyl 2,2-dimethyl-4-(5-(4-(piperidine-1-carboxamido)phenyl)thiazol-2-yl)butanoate;
2,2-Dimethyl-4-(5-(4-(piperidine-1-carboxamido)phenyl)thiazol-2-yl)butanoic acid;
Methyl 2,2-dimethyl-4-(5-(4-(morpholine-4-carboxamido)phenyl)thiazol-2-yl)butanoate;
2,2-Dimethyl-4-(5-(4-(morpholine-4-carboxamido)phenyl)thiazol-2-yl)butanoic acid;
Methyl 2,2-dimethyl-4-(5-(4-(4-methylpiperazine-1-carboxamido)phenyl)thiazol-2-yl)butanoate;
2,2-Dimethyl-4-(5-(4-(4-methylpiperazine-1-carboxamido)phenyl)thiazol-2-yl)butanoic acid hydrochloride;
Methyl 4-(5-(4-(3-(2,3-dihydrobenzo[b][1,4]dioxin-6-yl)ureido)phenyl)thiazol-2-yl)-2,2-dimethylbutanoate;
4-(5-(4-(3-(2,3-Dihydrobenzo[b][1,4]dioxin-6-yl)ureido)phenyl)thiazol-2-yl)-2,2-dimethylbutanoic acid;
Methyl 4-(5-(4-(3-(1H-tetrazol-5-yl)ureido)phenyl)thiazol-2-yl)-2,2-dimethyl butanoate;
4-(5-(4-(3-(1H-Tetrazol-5-yl)ureido)phenyl)thiazol-2-yl)-2,2-dimethylbutanoic acid;
Methyl 4-(5-(4-(3-(2-methoxyethyl)ureido)phenyl)thiazol-2-yl)-2,2-dimethyl butanoate;
4-(5-(4-(3-(2-Methoxyethyl)ureido)phenyl)thiazol-2-yl)-2,2-dimethylbutanoic acid;
Methyl 4-(5-(4-(3-(2,3-dihydro-1H-inden-2-yl)ureido)phenyl)thiazol-2-yl)-2,2-dimethylbutanoate;
4-(5-(4-(3-(2,3-Dihydro-1H-inden-2-yl)ureido)phenyl)thiazol-2-yl)-2,2-dimethyl butanoic acid;
Methyl 4-(5-(4-(3-cyclohexyl-3-methylureido)phenyl)thiazol-2-yl)-2,2-dimethyl butanoate;
4-(5-(4-(3-Cyclohexyl-3-methylureido)phenyl)thiazol-2-yl)-2,2-dimethylbutanoic acid;
Methyl 2,2-dimethyl-4-(5-(4-(3-(3,4,5-trifluorophenyl)ureido)phenyl)thiazol-2-yl)butanoate;
2,2-Dimethyl-4-(5-(4-(3-(3,4,5-trifluorophenyl)ureido)phenyl)thiazol-2-yl)butanoic acid;
Sodium salt of 2,2-dimethyl-4-(5-(4-(3-(3,4,5-trifluorophenyl)ureido)phenyl)thiazol-2-yl)butanoate;
Methyl 2,2-dimethyl-4-(5-(4-(3-(2-(piperidin-1-yl)ethyl)ureido)phenyl)thiazol-2-yl)butanoate;
2,2-Dimethyl-4-(5-(4-(3-(2-(piperidin-1-yl)ethyl)ureido)phenyl)thiazol-2-yl)butanoic acid;
Methyl 4-(5-(4-(3-benzylureido)phenyl)thiazol-2-yl)-2,2-dimethylbutanoate;
4-(5-(4-(3-Benzylureido)phenyl)thiazol-2-yl)-2,2-dimethylbutanoic acid;
Methyl 4-(5-(4-(4,4-difluoropiperidine-1-carboxamido)phenyl)thiazol-2-yl)-2,2-dimethylbutanoate;
4-(5-(4-(4,4-Difluoropiperidine-1-carboxamido)phenyl)thiazol-2-yl)-2,2-dimethylbutanoic acid;
Methyl 2,2-dimethyl-4-(5-(4-(4-phenylpiperidine-1-carboxamido)phenyl)thiazol-2-yl)butanoate;
2,2-Dimethyl-4-(5-(4-(4-phenylpiperidine-1-carboxamido)phenyl)thiazol-2-yl)butanoic acid;
Methyl 2,2-dimethyl-4-(5-(4-(4-phenylpiperidine-1-carboxamido)phenyl)thiazol-2-yl)butanoate;
4-(5-(4-(3-(4-Cyanobenzyl)ureido)phenyl)thiazol-2-yl)-2,2-dimethylbutanoic acid;
Methyl 4-(5-(4-(3-(2-fluorophenyl)thioureido)phenyl)thiazol-2-yl)-2,2-dimethyl butanoate;
4-(5-(4-(3-(2-Fluorophenyl)thioureido)phenyl)thiazol-2-yl)-2,2-dimethylbutanoic acid;
Methyl 4-(5-(4-(3-(2-fluorophenyl)guanidino)phenyl)thiazol-2-yl)-2,2-dimethyl butanoate;
4-(5-(4-(3-(2-Fluorophenyl)guanidino)phenyl)thiazol-2-yl)-2,2-dimethylbutanoic acid;
Methyl 4-(5-(4-(3-(2-fluorophenyl)-2-methylguanidino)phenyl)thiazol-2-yl)-2,2-dimethylbutanoate;
4-(5-(4-(3-(2-Fluorophenyl)-2-methylguanidino)phenyl)thiazol-2-yl)-2,2-dimethylbutanoic acid;
Methyl 4-(5-(4-(2-cyano-3-(2-fluorophenyl)guanidino)phenyl)thiazol-2-yl)-2,2-dimethylbutanoate;
4-(5-(4-(2-Cyano-3-(2-fluorophenyl)guanidino)phenyl)thiazol-2-yl)-2,2-dimethyl butanoic acid;
Methyl 4-(5-(4-(3-(3-(trifluoromethyl)phenyl)ureido)phenyl)-1,3,4-thiadiazol-2-yl)butanoate;
4-(5-(4-(3-(3-(Trifluoromethyl)phenyl)ureido)phenyl)-1,3,4-thiadiazol-2-yl)butanoic acid;
Methyl 4-(5-(4-(3-(2-chlorophenyl)ureido)phenyl)-1,3,4-thiadiazol-2-yl)butanoate;
4-(5-(4-(3-(2-Chlorophenyl)ureido)phenyl)-1,3,4-thiadiazol-2-yl)butanoic acid;
Methyl 4-(5-(4-(3-(p-tolyl)ureido)phenyl)-1,3,4-thiadiazol-2-yl)butanoate;
4-(5-(4-(3-(p-Tolyl)ureido)phenyl)-1,3,4-thiadiazol-2-yl)butanoic acid;
Methyl 4-(5-(4-(3-(2,4-difluorophenyl)ureido)phenyl)-1,3,4-thiadiazol-2-yl)butanoate;
4-(5-(4-(3-(2,4-Difluorophenyl)ureido)phenyl)-1,3,4-thiadiazol-2-yl)butanoic acid;
Methyl 4-(5-(4-(3-(4-chloro-2-phenoxyphenyl)ureido)phenyl)-1,3,4-thiadiazol-2-yl)butanoate;
4-(5-(4-(3-(4-Chloro-2-phenoxyphenyl)ureido)phenyl)-1,3,4-thiadiazol-2-yl)butanoic acid;
Methyl 4-(5-(4-(4-(tert-butyl)benzamido)phenyl)-1,3,4-thiadiazol-2-yl)butanoate;
4-(5-(4-(4-(t-Butyl)benzamido)phenyl)-1,3,4-thiadiazol-2-yl)butanoic acid;
Methyl 4-(5-(4-([1,1′-biphenyl]-4-ylcarboxamido)phenyl)-1,3,4-thiadiazol-2-yl)butanoate;
4-(5-(4-([1,1′-Biphenyl]-4-ylcarboxamido)phenyl)-1,3,4-thiadiazol-2-yl)butanoic acid;
Methyl 4-(5-(4-(4-(trifluoromethoxy)benzamido)phenyl)-1,3,4-thiadiazol-2-yl)butanoate;
4-(5-(4-(4-(Trifluoromethoxy)benzamido)phenyl)-1,3,4-thiadiazol-2-yl)butanoic acid;
Methyl 4-(5-(4-(3-(2-chlorophenyl)ureido)phenyl)-1,3,4-oxadiazol-2-yl)butanoate;
4-(5-(4-(3-(2-Chlorophenyl)ureido)phenyl)-1,3,4-oxadiazol-2-yl)butanoic acid;
Methyl 4-(5-(4-(3-(m-tolyl)ureido)phenyl)-1,3,4-oxadiazol-2-yl)butanoate;
4-(5-(4-(3-(m-Tolyl)ureido)phenyl)-1,3,4-oxadiazol-2-yl)butanoic acid;
Methyl 4-(5-(4-(3-(2,4-difluorophenyl)ureido)phenyl)-1,3,4-oxadiazol-2-yl)butanoate;
4-(5-(4-(3-(2,4-Difluorophenyl)ureido)phenyl)-1,3,4-oxadiazol-2-yl)butanoic acid;
Methyl 4-(5-(4-(3-(3-(trifluoromethyl)phenyl)ureido)phenyl)-1,3,4-oxadiazol-2-yl) butanoate;
4-(5-(4-(3-(3-(Trifluoromethyl)phenyl)ureido)phenyl)-1,3,4-oxadiazol-2-yl)butanoic acid;
Ethyl 4-(3-(4-(3-(2-chlorophenyl)ureido)phenyl)-1H-pyrazol-1-yl)cyclohexane carboxylate;
4-(3-(4-(3-(2-Chlorophenyl)ureido)phenyl)-1H-pyrazol-1-yl)cyclohexane carboxylic acid;
Ethyl 4-(3-(4-(3-(2-fluorophenyl)ureido)phenyl)-1H-pyrazol-1-yl)cyclohexane carboxylate;
4-(3-(4-(3-(2-Fluorophenyl)ureido)phenyl)-1H-pyrazol-1-yl)cyclohexane carboxylic acid;
Ethyl 4-(3-(4-(3-(2,4-difluorophenyl)ureido)phenyl)-1H-pyrazol-1-yl)cyclohexane carboxylate;
4-(3-(4-(3-(2,4-Difluorophenyl)ureido)phenyl)-1H-pyrazol-1-yl)cyclohexane carboxylic acid;
Ethyl 4-(3-(4-(3-(3-(trifluoromethyl)phenyl)ureido)phenyl)-1H-pyrazol-1-yl)cyclohexanecarboxylate;
4-(3-(4-(3-(3-(Trifluoromethyl)phenyl)ureido)phenyl)-1H-pyrazol-1-yl)cyclohexanecarboxylic acid;
Ethyl 4-(3-(4-(3-(m-tolyl)ureido)phenyl)-1H-pyrazol-1-yl)cyclohexane carboxylate;
4-(3-(4-(3-(m-Tolyl)ureido)phenyl)-1H-pyrazol-1-yl)cyclohexanecarboxylic acid;
Methyl 4-(3-(4-(3-(4-chloro-2-phenoxyphenyl)ureido)phenyl)-1,2,4-oxadiazol-5-yl)butanoate;
4-(3-(4-(3-(4-Chloro-2-phenoxyphenyl)ureido)phenyl)-1,2,4-oxadiazol-5-yl)butanoic acid;
Methyl 4-(3-(4-(3-(2,4-difluorophenyl)ureido)phenyl)-1,2,4-oxadiazol-5-yl)butanoate;
4-(3-(4-(3-(2,4-Difluorophenyl)ureido)phenyl)-1,2,4-oxadiazol-5-yl)butanoic acid;
Methyl 4-(3-(4-(3-(2-chlorophenyl)ureido)phenyl)-1,2,4-oxadiazol-5-yl)butanoate;
4-(3-(4-(3-(2-Chlorophenyl)ureido)phenyl)-1,2,4-oxadiazol-5-yl)butanoic acid;
Methyl 4-(3-(4-(3-(4-chloro-2-phenoxyphenyl)ureido)phenyl)-1,2,4-oxadiazol-5-yl)butanoate;
4-(3-(4-(3-(4-Chloro-2-phenoxyphenyl)ureido)phenyl)-1,2,4-oxadiazol-5-yl)butanoic acid;
Methyl 4-(3-(4-(3-(2,4-difluorophenyl)ureido)phenyl)-1,2,4-oxadiazol-5-yl)butanoate;
4-(3-(4-(3-(2,4-Difluorophenyl)ureido)phenyl)-1,2,4-oxadiazol-5-yl)butanoic acid;
Methyl 4-(3-(4-(3-(2-chlorophenyl)ureido)phenyl)-1,2,4-oxadiazol-5-yl)butanoate;
4-(3-(4-(3-(2-Chlorophenyl)ureido)phenyl)-1,2,4-oxadiazol-5-yl)butanoic acid;
Methyl 4-(3-(4-(4-fluorobenzamido)phenyl)-1,2,4-oxadiazol-5-yl)-2,2-dimethyl butanoate;
4-(3-(4-(4-Fluorobenzamido)phenyl)-1,2,4-oxadiazol-5-yl)-2,2-dimethylbutanoic acid;
Methyl 4-(3-(4-([1,1′-biphenyl]-4-ylcarboxamido)phenyl)-1,2,4-oxadiazol-5-yl)-2,2-dimethylbutanoate;
4-(3-(4-([1,1′-Biphenyl]-4-ylcarboxamido)phenyl)-1,2,4-oxadiazol-5-yl)-2,2-dimethylbutanoic acid;
t-Butyl 2-(4-(5-(4-(3-(2-chlorophenyl)ureido)phenyl)thiazol-2-yl)cyclohexyl)acetate;
2-(4-(5-(4-(3-(2-Chlorophenyl)ureido)phenyl)thiazol-2-yl)cyclohexyl)acetic acid;
t-Butyl 2-(4-(5-(4-(3-(2-fluorophenyl)ureido)phenyl)thiazol-2-yl)cyclohexyl)acetate;
2-(4-(5-(4-(3-(2-Fluorophenyl)ureido)phenyl)thiazol-2-yl)cyclohexyl)acetic acid;
Ethyl 2-(4-(5-(4-(3-(3,5-difluorophenyl)ureido)phenyl)thiazol-2-yl)cyclohexyl)acetate;
2-(4-(5-(4-(3-(3,5-Difluorophenyl)ureido)phenyl)thiazol-2-yl)cyclohexyl)acetic acid;
Ethyl 2-(4-(5-(4-(3-(2,4,5-trifluorophenyl)ureido)phenyl)thiazol-2-yl)cyclohexyl)acetate;
2-(4-(5-(4-(3-(2,4,5-Trifluorophenyl)ureido)phenyl)thiazol-2-yl)cyclohexyl)acetic acid;
Ethyl 2-(4-(5-(4-(3-(2,4,6-trifluorophenyl)ureido)phenyl)thiazol-2-yl)cyclohexyl)acetate;
2-(4-(5-(4-(3-(2,4,6-Trifluorophenyl)ureido)phenyl)thiazol-2-yl)cyclohexyl)acetic acid;
Ethyl 2-(4-(5-(4-(3-(2,4-difluorophenyl)ureido)phenyl)thiazol-2-yl)cyclohexyl)acetate;
2-(4-(5-(4-(3-(2,4-Difluorophenyl)ureido)phenyl)thiazol-2-yl)cyclohexyl)acetic acid;
Ethyl 2-(4-(5-(4-(2,4-dichlorobenzamido)phenyl)thiazol-2-yl)cyclohexyl)acetate;
2-(4-(5-(4-(2,4-Dichlorobenzamido)phenyl)thiazol-2-yl)cyclohexyl)acetic acid;
Ethyl 2-(4-(5-(4-(2-fluoro-6-(trifluoromethyl)benzamido)phenyl)thiazol-2-yl)cyclohexyl)acetate;
2-(4-(5-(4-(2-Fluoro-6-(trifluoromethyl)benzamido)phenyl)thiazol-2-yl)cyclohexyl)acetic acid;
Ethyl 2-(4-(5-(4-(3-(3,4,5-trifluorophenyl)ureido)phenyl)thiazol-2-yl)cyclohexyl)acetate;
2-(4-(5-(4-(3-(3,4,5-Trifluorophenyl)ureido)phenyl)thiazol-2-yl)cyclohexyl)acetic acid;
Ethyl 2-(4-(5-(4-(2-phenyl-5-(trifluoromethyl)oxazole-4-carboxamido)phenyl)thiazol-2-yl)cyclohexyl)acetate;
2-(4-(5-(4-(2-Phenyl-5-(trifluoromethyl)oxazole-4-carboxamido)phenyl)thiazol-2-yl)cyclohexyl)acetic acid;
Ethyl 2-(4-(5-(4-(5-methyl-2-phenyloxazole-4-carboxamido)phenyl)thiazol-2-yl)cyclohexyl)acetate;
2-(4-(5-(4-(5-Methyl-2-phenyloxazole-4-carboxamido)phenyl)thiazol-2-yl)cyclohexyl)acetic acid; Ethyl 2-(4-(5-(4-(3-(2-fluorophenyl)thioureido)phenyl)thiazol-2-yl)cyclohexyl)acetate;
2-(4-(5-(4-(3-(2-Fluorophenyl)thioureido)phenyl)thiazol-2-yl)cyclohexyl)acetic acid;
Ethyl 2-(4-(5-(4-(3-(2-fluorophenyl)guanidino)phenyl)thiazol-2-yl)cyclohexyl)acetate;
4-(2-(4-((5-Methyl-1,3,4-oxadiazol-2-yl-methyl)cyclohexyl)thiazol-5-yl)aniline;
1-(2,4-Difluorophenyl)-3-(4-(2-(4-((5-methyl-1,3,4-oxadiazol-2-yl)methyl)cyclohexyl)thiazol-5-yl)phenyl)urea;
1-(2-Chlorophenyl)-3-(4-(2-(4-((5-methyl-1,3,4-oxadiazol-2-yl)methyl)cyclohexyl)thiazol-5-yl)phenyl)urea;
1-(3,5-Difluorophenyl)-3-(4-(2-(4-((5-methyl-1,3,4-oxadiazol-2-yl)methyl)cyclohexyl)thiazol-5-yl)phenyl)urea;
1-(4-(2-(4-((5-Methyl-1,3,4-oxadiazol-211)methyl)cyclohexyl)thiazol-5-yl)phenyl)-3-(2,4,5-trifluorophenyl)urea;
1-(4-(2-(4-((5-Methyl-1,3,4-oxadiazol-211)methyl)cyclohexyl)thiazol-5-yl)phenyl)-3-(2,4,6-trifluorophenyl)urea;
1-(4-(2-(4-((5-Methyl-1,3,4-oxadiazol-211)methyl)cyclohexyl)thiazol-5-yl)phenyl)-3-phenylurea;
2,6-Difluoro-N-(4-(2-(4-((5-methyl-1,3,4-oxadiazol-2-yl)methyl)cyclohexyl)thiazol-5-yl)phenyl)benzamide;
4-(2-(4-((3-Methyl-1,2,4-oxadiazol-5-yl)methyl)cyclohexyl)thiazol-5-yl)aniline;
1-(2-Chlorophenyl)-3-(4-(2-(4-((3-methyl-1,2,4-oxadiazol-5-yl)methyl)cyclohexyl)thiazol-5-yl)phenyl)urea;
1-(2-Fluorophenyl)-3-(4-(2-(4-((3-methyl-1,2,4-oxadiazol-5-yl)methyl)cyclohexyl)thiazol-5-yl)phenyl)urea;
1-(3,5-Difluorophenyl)-3-(4-(2-(4-((3-methyl-1,2,4-oxadiazol-5-yl)methyl)cyclohexyl)thiazol-5-yl)phenyl)urea;
1-(4-(2-(4-((3-Methyl-1,2,4-oxadiazol-511)methyl)cyclohexyl)thiazol-5-yl)phenyl)-3-(2,4,5-trifluorophenyl)urea;
1-(2,4-Difluorophenyl)-3-(4-(2-(4-((3-methyl-1,2,4-oxadiazol-5-yl)methyl)cyclohexyl)thiazol-5-yl)phenyl)urea;
1-(4-(2-(4-((3-Methyl-1,2,4-oxadiazol-5-yl)methyl)cyclohexyl)thiazol-5-yl)phenyl)-3-phenylurea;
2,6-Difluoro-N-(4-(2-(4-((3-methyl-1,2,4-oxadiazol-5-yl)methyl)cyclohexyl)thiazol-5-yl)phenyl)benzamide;
2-Chloro-N-(4-(2-(4-((3-methyl-1,2,4-oxadiazol-5-yl)methyl)cyclohexyl)thiazol-5-yl)phenyl)benzamide;
3,5-Difluoro-N-(4-(2-(4-((3-methyl-1,2,4-oxadiazol-5-yl)methyl)cyclohexyl)thiazol-5-yl)phenyl)benzamide;
N-Acetyl-2-(4-(5-(4-aminophenyl)thiazol-2-yl)cyclohexyl)acetamide;
N-Acetyl-2-(4-(5-(4-(3-(2-chlorophenyl)ureido)phenyl)thiazol-2-yl)cyclohexyl)acetamide;
N-Acetyl-2-(4-(5-(4-(3-(2,4-difluorophenyl)ureido)phenyl)thiazol-2-yl)cyclohexyl)acetamide;
N-Acetyl-2-(4-(5-(4-(3-(2,4,5-trifluorophenyl)ureido)phenyl)thiazol-2-yl)cyclohexyl)acetamide;
N-(4-(2-(4-(2-Acetamido-2-oxoethyl)cyclohexyl)thiazol-5-yl)phenyl)-2,6-difluoro benzamide;
1-(2-Chlorophenyl)-3-(4-(2-(4-(2-hydroxypropan-2-yl)cyclohexyl)thiazol-5-yl)phenyl)urea;
1-(3,5-Difluorophenyl)-3-(4-(2-(4-(2-hydroxypropan-2-yl)cyclohexyl)thiazol-5-yl)phenyl)urea;
1-(2,4-Difluorophenyl)-3-(4-(2-(4-(2-hydroxypropan-2-yl)cyclohexyl)thiazol-5-yl)phenyl)urea;
1-(2,4-Difluorophenyl)-3-(4-(2-(4-(2-hydroxy-2-methylpropyl)cyclohexyl)thiazol-5-yl)phenyl)urea;
1-(3,5-Difluorophenyl)-3-(4-(2-(4-(2-hydroxy-2-methylpropyl)cyclohexyl)thiazol-5-yl)phenyl)urea;
1-(4-(2-(4-(2-Hydroxy-2-methylpropyl)cyclohexyl)thiazol-5-yl)phenyl)-3-(2,4,5-trifluorophenyl)urea;
1-(3,5-Difluorophenyl)-3-(4-(2-(4-(2-hydrazinyl-2-oxoethyl)cyclohexyl)thiazol-5-yl)phenyl)urea;
4-(2-(4-((5-Methyl-1,3,4-thiadiazol-2-yl)methyl)cyclohexyl)thiazol-5-yl)aniline;
1-(4-(2-(4-((5-Methyl-1,3,4-thiadiazol-2-yl)methyl)cyclohexyl)thiazol-5-yl)phenyl)-3-(2,4,5-trifluorophenyl)urea;
Ethyl 2-(4-(4-(4-(3-(2-fluorophenyl)ureido)phenyl)thiazol-2-yl)piperidin-1-yl)acetate;
2-(4-(4-(4-(3-(2-Fluorophenyl)ureido)phenyl)thiazol-2-yl)piperidin-1-yl)acetic acid;
Ethyl 2-(4-(4-(4-(3-(2-chlorophenyl)ureido)phenyl)thiazol-2-yl)piperidin-1-yl)acetate;
2-(4-(4-(4-(3-(2-Chlorophenyl)ureido)phenyl)thiazol-2-yl)piperidin-1-yl)acetic acid;
Ethyl 2-(4-(5-(4-(3-(2-chlorophenyl)ureido)phenyl)thiazol-2-yl)piperidin-1-yl)acetate;
2-(4-(5-(4-(3-(2-Chlorophenyl)ureido)phenyl)thiazol-2-yl)piperidin-1-yl)acetic acid;
Ethyl 2-(4-(5-(4-(3-(2-fluorophenyl)ureido)phenyl)thiazol-2-yl)piperidin-1-yl)acetate;
2-(4-(5-(4-(3-(2-Fluorophenyl)ureido)phenyl)thiazol-2-yl)piperidin-1-yl)acetic acid;
Ethyl 2-(4-(5-(4-(3-(2,4-difluorophenyl)ureido)phenyl)thiazol-2-yl)piperidin-1-yl)acetate;
2-(4-(5-(4-(3-(2,4-Difluorophenyl)ureido)phenyl)thiazol-2-yl)piperidin-1-yl)acetic acid;
Ethyl 2-(4-(5-(4-(3-(2,4,5-trifluorophenyl)ureido)phenyl)thiazol-2-yl)piperidin-1-yl)acetate;
2-(4-(5-(4-(3-(2,4,5-Trifluorophenyl)ureido)phenyl)thiazol-2-yl)piperidin-1-yl)acetic acid;
Ethyl 2-(4-(5-(4-(3-(2-(trifluoromethyl)phenyl)ureido)phenyl)thiazol-2-yl)piperidin-1-yl)acetate;
2-(4-(5-(4-(3-(2-(Trifluoromethyl)phenyl)ureido)phenyl)thiazol-2-yl)piperidin-1-yl)acetic acid;
Ethyl 2-(4-(5-(4-(3-(2,3,4-trifluorophenyl)ureido)phenyl)thiazol-2-yl)piperidin-1-yl)acetate;
2-(4-(5-(4-(3-(2,3,4-Trifluorophenyl)ureido)phenyl)thiazol-2-yl)piperidin-1-yl)acetic acid;
Ethyl 2-(4-(5-(4-(3-(2,4,6-trifluorophenyl)ureido)phenyl)thiazol-2-yl)piperidin-1-yl)acetate;
2-(4-(5-(4-(3-(2,4,6-Trifluorophenyl)ureido)phenyl)thiazol-2-yl)piperidin-1-yl)acetic acid;
Ethyl 2-methyl-2-(4-(5-(4-(3-(2,4,5-trifluorophenyl)ureido)phenyl)thiazol-2-yl)piperidin-1-yl)propanoate;
Ethyl 2-(4-(5-(4-(3-(2-fluorophenyl)ureido)phenyl)thiazol-2-yl)piperidin-1-yl)-2-methylpropanoate;
Ethyl 2-(4-(5-(4-(3-(2-chlorophenyl)ureido)phenyl)thiazol-2-yl)piperidin-1-yl)-2-methylpropanoate;
Ethyl 2-(4-(5-(4-(3-(2,4-difluorophenyl)ureido)phenyl)thiazol-2-yl)piperidin-1-yl)-2-methylpropanoate;
t-Butyl 2-(4-(5-(4-(3-(2,4,5-trifluorophenyl)ureido)phenyl)thiazol-2-yl)piperidin-1-yl)propanoate;
2-(4-(5-(4-(3-(2,4,5-Trifluorophenyl)ureido)phenyl)thiazol-2-yl)piperidin-1-yl)propanoic acid;
t-Butyl 2-(4-(5-(4-(3-(2-fluorophenyl)ureido)phenyl)thiazol-2-yl)piperidin-1-yl)propanoate;
2-(4-(5-(4-(3-(2-Fluorophenyl)ureido)phenyl)thiazol-2-yl)piperidin-1-yl)propanoic acid;
t-Butyl 2-(4-(5-(4-(3-(2-chlorophenyl)ureido)phenyl)thiazol-2-yl)piperidin-1-yl)propanoate;
2-(4-(5-(4-(3-(2-Chlorophenyl)ureido)phenyl)thiazol-2-yl)piperidin-1-yl)propanoic acid;
t-Butyl 2-(4-(5-(4-(3-(2,4-difluorophenyl)ureido)phenyl)thiazol-2-yl)piperidin-1-yl)propanoate;
2-(4-(5-(4-(3-(2,4-Difluorophenyl)ureido)phenyl)thiazol-2-yl)piperidin-1-yl)propanoic acid;
t-Butyl 2-(4-(5-(4-(3-(2,4,6-trifluorophenyl)ureido)phenyl)thiazol-2-yl)piperidin-1-yl)propanoate;
2-(4-(5-(4-(3-(2,4,6-Trifluorophenyl)ureido)phenyl)thiazol-2-yl)piperidin-1-yl)propanoic acid;
t-Butyl 2-methyl-2-(4-(5-(4-(3-(2,4,5-trifluorophenyl)ureido)phenyl)thiazol-2-yl)piperidin-1-yl)propanoate;
2-Methyl-2-(4-(5-(4-(3-(2,4,5-trifluorophenyl)ureido)phenyl)thiazol-2-yl)piperidin-1-yl)propanoic acid;
t-Butyl 2-(4-(5-(4-(3-(2-fluorophenyl)ureido)phenyl)thiazol-2-yl)piperidin-1-yl)-2-methylpropanoate;
2-(4-(5-(4-(3-(2-Fluorophenyl)ureido)phenyl)thiazol-2-yl)piperidin-1-yl)-2-methylpropanoic acid;
t-Butyl 2-(4-(5-(4-(3-(2-chlorophenyl)ureido)phenyl)thiazol-2-yl)piperidin-1-yl)-2-methylpropanoate;
2-(4-(5-(4-(3-(2-Chlorophenyl)ureido)phenyl)thiazol-2-yl)piperidin-1-yl)-2-methyl propanoic acid;
t-Butyl 2-(4-(5-(4-(3-(2,4-difluorophenyl)ureido)phenyl)thiazol-2-yl)piperidin-1-yl)-2-methylpropanoate;
2-(4-(5-(4-(3-(2,4-Difluorophenyl)ureido)phenyl)thiazol-2-yl)piperidin-1-yl)-2-methylpropanoic acid;
t-Butyl 2-methyl-2-(4-(5-(4-(3-(2,4,6-trifluorophenyl)ureido)phenyl)thiazol-2-yl)piperidin-1-yl)propanoate;
2-Methyl-2-(4-(5-(4-(3-(2,4,6-trifluorophenyl)ureido)phenyl)thiazol-2-yl)piperidin-1-yl)propanoic acid;
t-Butyl 4-(5-(4-(3-(2-chlorophenyl)ureido)phenyl)thiazol-2-yl)piperidine-1-carboxylate;
1-(2-Chlorophenyl)-3-(4-(2-(piperidin-4-yl)thiazol-5-yl)phenyl)urea hydrochloride;
t-Butyl 4-(5-(4-(3-(2-fluorophenyl)ureido)phenyl)thiazol-2-yl)piperidine-1-carboxylate;
1-(2-Fluorophenyl)-3-(4-(2-(piperidin-4-yl)thiazol-5-yl)phenyl)urea hydrochloride;
t-Butyl 4-(5-(4-(3-(2,4-difluorophenyl)ureido)phenyl)thiazol-2-yl)piperidine-1-carboxylate;
1-(2,4-Difluorophenyl)-3-(4-(2-(piperidin-4-yl)thiazol-5-yl)phenyl)urea hydrochloride;
t-Butyl 4-(5-(4-(3-(2,4,6-trifluorophenyl)ureido)phenyl)thiazol-2-yl)piperidine-1-carboxylate;
1-(4-(2-(Piperidin-4-yl)thiazol-5-yl)phenyl)-3-(2,4,5-trifluorophenyl)urea hydrochloride;
1-(2-Fluorophenyl)-3-(4-(2-(1-((trifluoromethyl)sulfonyl)piperidin-4-yl)thiazol-5-yl)phenyl)urea;
1-(2-Chlorophenyl)-3-(4-(2-(1-((trifluoromethyl)sulfonyl)piperidin-4-yl)thiazol-5-yl)phenyl)urea;
1-(2,4-Difluorophenyl)-3-(4-(2-(1-((trifluoromethyl)sulfonyl)piperidin-4-yl)thiazol-5-yl)phenyl)urea;
1-(4-(2-(1-((Trifluoromethyl)sulfonyl)piperidin-4-yl)thiazol-5-yl)phenyl)-3-(2,4,6-trifluorophenyl)urea;
1-(4-(2-(1-((Trifluoromethyl)sulfonyl)piperidin-4-yl)thiazol-5-yl)phenyl)-3-(2,4,5-trifluorophenyl)urea;
1-(2-Chlorophenyl)-3-(4-(2-(1-(methylsulfonyl)piperidin-4-yl)thiazol-5-yl)phenyl)urea;
1-(2-Fluorophenyl)-3-(4-(2-(1-(methylsulfonyl)piperidin-4-yl)thiazol-5-yl)phenyl)urea;
1-(2,4-Difluorophenyl)-3-(4-(2-(1-(methylsulfonyl)piperidin-4-yl)thiazol-5-yl)phenyl)urea;
1-(4-(2-(1-(Methylsulfonyl)piperidin-4-yl)thiazol-5-yl)phenyl)-3-(2,4,6-trifluoro phenyl)urea;
1-(4-(2-(1-(Methylsulfonyl)piperidin-4-yl)thiazol-5-yl)phenyl)-3-(2,4,5-trifluoro phenyl)urea;
Methyl 3-(5-(4-(3-(2-chlorophenyl)ureido)phenyl)thiazol-2-yl)adamantane-1-carboxylate;
3-(5-(4-(3-(2-Chlorophenyl)ureido)phenyl)thiazol-2-yl)adamantane-1-carboxylic acid;
Methyl 3-(5-(4-(3-(2-fluorophenyl)ureido)phenyl)thiazol-2-yl)adamantane-1-carboxylate;
3-(5-(4-(3-(2-Fluorophenyl)ureido)phenyl)thiazol-2-yl)adamantane-1-carboxylic acid;
Methyl 3-(5-(4-(3-(2,4-difluorophenyl)ureido)phenyl)thiazol-2-yl)adamantine-1-carboxylate;
3-(5-(4-(3-(2,4-Difluorophenyl)ureido)phenyl)thiazol-2-yl)adamantane-1-carboxylic acid;
Methyl 3-(5-(4-(3-(2,6-difluorophenyl)ureido)phenyl)thiazol-2-yl)adamantine-1-carboxylate;
3-(5-(4-(3-(2,6-Difluorophenyl)ureido)phenyl)thiazol-2-yl)adamantane-1-carboxylic acid;
Methyl 3-(5-(4-(3-(2,4,5-trifluorophenyl)ureido)phenyl)thiazol-2-yl)adamantane-1-carboxylate;
3-(5-(4-(3-(2,4,5-Trifluorophenyl)ureido)phenyl)thiazol-2-yl)adamantane-1-carboxylic acid;
Methyl 3-(5-(4-(3-(2,3,4-trifluorophenyl)ureido)phenyl)thiazol-2-yl)adamantane-1-carboxylate;
3-(5-(4-(3-(2,3,4-Trifluorophenyl)ureido)phenyl)thiazol-2-yl)adamantane-1-carboxylic acid;
Methyl 3-(5-(4-(3-(3,5-difluorophenyl)ureido)phenyl)thiazol-2-yl)adamantane-1-carboxylate;
3-(5-(4-(3-(3,5-Difluorophenyl)ureido)phenyl)thiazol-2-yl)adamantane-1-carboxylic acid;
Methyl 3-(5-(4-(3-(3-(trifluoromethyl)phenyl)ureido)phenyl)thiazol-2-yl)adamantane-1-carboxylate;
3-(5-(4-(3-(3-(Trifluoromethyl)phenyl)ureido)phenyl)thiazol-2-yl)adamantane-1-carboxylic acid;
N-(2-(5-(4-(3-(2-Chlorophenyl)ureido)phenyl)thiazol-2-yl)ethyl)-1,1,1-trifluoro methanesulfonamide;
1,1,1-Trifluoro-N-(2-(5-(4-(3-(2-fluorophenyl)ureido)phenyl)thiazol-2-yl)ethyl)methanesulfonamide;
N-(2-(5-(4-(3-(3,5-Difluorophenyl)ureido)phenyl)thiazol-2-yl)ethyl)-1,1,1-trifluoromethanesulfonamide;
1,1,1-Trifluoro-N-(2-(5-(4-(3-(2,4,5-trifluorophenyl)ureido)phenyl)thiazol-2-yl)ethyl)methanesulfonamide;
1,1,1-Trifluoro-N-(2-(5-(4-(3-(2,4,6-trifluorophenyl)ureido)phenyl)thiazol-2-yl)ethyl)methanesulfonamide;
1,1,1-Trifluoro-N-(2-(5-(4-(3-(4-(trifluoromethyl)phenyl)ureido)phenyl)thiazol-2-yl)ethyl)methanesulfonamide;
1,1,1-Trifluoro-N-(2-(5-(4-(3-phenylureido)phenyl)thiazol-2-yl)ethyl)methane sulfonamide;
N-(2-(5-(4-(3-Cyclohexylureido)phenyl)thiazol-2-yl)ethyl)-1,1,1-trifluoro methanesulfonamide;
2-Chloro-N-(4-(2-(2-(trifluoromethylsulfonamido)ethyl)thiazol-5-yl)phenyl)benzamide;
N-(4-(2-(2-(Trifluoromethylsulfonamido)ethyl)thiazol-5-yl)phenyl)cyclohexane carboxamide;
4-(Trifluoromethyl)-N-(4-(2-(2-(trifluoromethylsulfonamido)ethyl)thiazol-5-yl)phenyl)benzamide;
N-(4-(2-(2-(Trifluoromethylsulfonamido)ethyl)thiazol-5-yl)phenyl)benzamide;
2-Phenyl-5-(trifluoromethyl)-N-(4-(2-(2-(trifluoromethylsulfonamido)ethyl)thiazol-5-yl)phenyl)oxazole-4-carboxamide;
1,1,1-Trifluoro-N-(2-(5-(4-(3-(2-fluorophenyl)thioureido)phenyl)thiazol-2-yl)ethyl)methanesulfonamide;
1,1,1-Trifluoro-N-(2-(5-(4-(3-(2-fluorophenyl)guanidino)phenyl)thiazol-2-yl)ethyl)methanesulfonamide;
1,1,1-Trifluoro-N-(2-(5-(4-(3-(2-fluorophenyl)-2-methylguanidino)phenyl)thiazol-2-yl)ethyl)methanesulfonamide;
N-(2-(5-(4-(2-Cyano-3-(2-fluorophenyl)guanidino)phenyl)thiazol-2-yl)ethyl)-1,1,1-trifluoromethanesulfonamide;
N-((5-(4-(3-(2-Chlorophenyl)ureido)phenyl)thiazol-2-yl)methyl)-1,1,1-trifluoro methanesulfonamide;
1,1,1-Trifluoro-N-((5-(4-(3-(2-fluorophenyl)ureido)phenyl)thiazol-2-yl)methyl)methanesulfonamide;
N-((5-(4-(3-(3,5-Difluorophenyl)ureido)phenyl)thiazol-2-yl)methyl)-1,1,1-trifluoromethanesulfonamide;
1,1,1-Trifluoro-N-((5-(4-(3-(2,4,5-trifluorophenyl)ureido)phenyl)thiazol-2-yl)methyl)methanesulfonamide;
1,1,1-Trifluoro-N-((5-(4-(3-(2,4,6-trifluorophenyl)ureido)phenyl)thiazol-2-yl)methyl)methanesulfonamide;
N-((5-(4-(3-Cyclohexylureido)phenyl)thiazol-2-yl)methyl)-1,1,1-trifluoro methanesulfonamide;
1,1,1-Trifluoro-N-((5-(4-(3-(4-(trifluoromethyl)phenyl)ureido)phenyl)thiazol-2-yl)methyl)methanesulfonamide;
1,1,1-Trifluoro-N-((5-(4-(3-phenylureido)phenyl)thiazol-2-yl)methyl)methane sulfonamide;
2-Chloro-N-(4-(2-((trifluoromethylsulfonamido)methyl)thiazol-5-yl)phenyl)benzamide;
4-(Trifluoromethyl)-N-(4-(2-((trifluoromethylsulfonamido)methyl)thiazol-5-yl)phenyl)benzamide;
N-(4-(2-((Trifluoromethylsulfonamido)methyl)thiazol-5-yl)phenyl)benzene sulfonamide;
4-(Trifluoromethyl)-N-(4-(2-((trifluoromethylsulfonamido)methyl)thiazol-5-yl)phenyl)benzenesulfonamide;
N-(4-(2-((Trifluoromethylsulfonamido)methyl)thiazol-5-yl)phenyl)cyclohexane sulfonamide;
2,4-Difluoro-N-(4-(2-((trifluoromethylsulfonamido)methyl)thiazol-5-yl)phenyl)benzenesulfonamide;
N-(2-(5-(4-(3-(2-Chlorophenyl)ureido)phenyl)thiazol-2-yl)propan-2-yl)-1,1,1-trifluoromethanesulfonamide;
1,1,1-Trifluoro-N-(2-(5-(4-(3-(2-fluorophenyl)ureido)phenyl)thiazol-2-yl)propan-2-yl)methanesulfonamide;
N-(2-(5-(4-(3-(3,5-Difluorophenyl)ureido)phenyl)thiazol-2-yl)propan-2-yl)-1,1,1-trifluoromethanesulfonamide;
1,1,1-Trifluoro-N-(2-(5-(4-(3-(2,4,5-trifluorophenyl)ureido)phenyl)thiazol-2-yl)propan-2-yl)methanesulfonamide;
1,1,1-Trifluoro-N-(2-(5-(4-(3-(2,4,6-trifluorophenyl)ureido)phenyl)thiazol-2-yl)propan-2-yl)methanesulfonamide;
N-(2-(5-(4-(3-Cyclohexylureido)phenyl)thiazol-2-yl)propan-2-yl)-1,1,1-trifluoromethanesulfonamide;
N-(4-(2-(2-(Trifluoromethylsulfonamido)propan-2-yl)thiazol-5-yl)phenyl)benzenesulfonamide;
t-Butyl(2-(5-(4-(3-(2-chlorophenyl)ureido)phenyl)thiazol-2-yl)ethyl)carbamate;
t-Butyl (2-(5-(4-(3-(3,5-difluorophenyl)ureido)phenyl)thiazol-2-yl)ethyl)carbamate;
t-Butyl (2-(5-(4-(3-(2,4,5-trifluorophenyl)ureido)phenyl)thiazol-2-yl)ethyl)carbamate;
1-(4-(2-(2-Aminoethyl)thiazol-5-yl)phenyl)-3-(2-chlorophenyl)urea hydrochloride;
1-(4-(2-(2-Aminoethyl)thiazol-5-yl)phenyl)-3-(3,5-difluorophenyl)urea hydrochloride;
1-(4-(2-(2-Aminoethyl)thiazol-5-yl)phenyl)-3-(2,4,5-trifluorophenyl)urea hydrochloride;
4-(5-(4-(3-(2-Chlorophenyl)ureido)phenyl)thiazol-2-yl)-2,2-dimethyl-N-((trifluoromethyl)sulfonyl)butanamide;
4-(5-(4-(3-(2-Fluorophenyl)ureido)phenyl)thiazol-2-yl)-2,2-dimethyl-N-((trifluoromethyl)sulfonyl)butanamide;
4-(5-(4-(3-(3,5-Difluorophenyl)ureido)phenyl)thiazol-2-yl)-2,2-dimethyl-N-((trifluoro methyl)sulfonyl)butanamide;
2,2-Dimethyl-N-((trifluoromethyl)sulfonyl)-4-(5-(4-(3-(2,4,5-trifluorophenyl)ureido)phenyl)thiazol-2-yl)butanamide;
Methyl 4-(5-(4-(3-(2,4,5-trifluorophenyl)ureido)phenyl)thiazol-2-yl)cyclohexane carboxylate;
4-(5-(4-(3-(2,4,5-Trifluorophenyl)ureido)phenyl)thiazol-2-yl)cyclohexanecarboxylic acid;
1-(4-(2-(4-(2-Hydroxypropan-2-yl)cyclohexyl)thiazol-5-yl)phenyl)-3-(2,4,5-trifluorophenyl)urea;
1-(4-(2-(4-(2-Aminopropan-2-yl)cyclohexyl)thiazol-5-yl)phenyl)-3-(2,4,5-trifluoro phenyl)urea;
1-(4-(2-(4-(2-Aminopropan-2-yl)cyclohexyl)thiazol-5-yl)phenyl)-3-(2,4-difluoro phenyl)urea; or
1-(4-(2-(4-(2-Amino-2-methylpropyl)cyclohexyl)thiazol-5-yl)phenyl)-3-(2,4,5-trifluoro phenyl)urea;
   in all their stereoisomeric and tautomeric forms; and their pharmaceutically acceptable salts, solvates, polymorphs, prodrugs, carboxylic acid isosteres and N-oxides.

The compounds of the present invention also include all stereoisomeric and tautomeric forms and mixtures thereof in all ratios and their pharmaceutically acceptable salts, solvates, polymorphs, prodrugs, carboxylic acid isosteres and N-oxides.

According to another aspect of present invention, a compound of formula 1 can be prepared in a number of ways including using methods well known to the person skilled in the art. Examples of methods to prepare the present compounds are described below and illustrated in Schemes 1 to 27, but not limited thereto. It will be appreciated by persons skilled in the art that within certain of the processes described herein, the order of the synthetic steps employed may be varied and will depend inter alia on factors such as the nature of functional groups present in a particular substrate and the protecting group strategy (if any) to be adopted. Clearly, such factors will also influence the choice of reagent to be used in the synthetic steps.

The reagents, reactants and intermediates used in the following processes are either commercially available or can be prepared according to standard literature procedures known in the art. The starting compounds and the intermediates used for the synthesis of compounds of the present invention are referred to numerically (Examples 1 to 591).

Throughout the process description, the corresponding substituent groups in the various formulae representing starting compounds and intermediates have the same meanings as that for the compound of formula 1 unless stated otherwise.

The schemes of the present invention are referred to numerically (1A to 1D; 2A to 2D; 3A to 3D; 4A to 4D; 5A to 5D; 6A to 6D; 7A to 7D, 8A to 8D; 9A to 9D; 10A to 10D; 11A to 11D; 12A to 12D; 13A to 13D, 14A to 14D and 15 to 27). The processes used in various schemes of the present invention, are referred to with general symbols such as 1a to 1p, 2a to 2k, 3a to 3m, 4a to 4p, 5a to 5n, 6a to 6k, 7a to 7m, 8a to 8m, 9a to 9k, 10a to 10k, 11a to 11n, 12a to 12m, 13a to 13m, 14a to 14k, 15a to 15e, 16a to 16j, 17a to 17e, 18a to 18d, 19a to 19m, 20a to 20g, 21a to 21f, 22a to 22h, 23a to 23f, 24a to 24e, 25a to 25h, 26a to 26f and 27a to 27b. Processes for the preparation of compounds of the present invention are set forth in the following schemes:

### Scheme 1A:

Scheme 1A depicts a process for the preparation of the compounds of formula 1 (referred in Scheme 1A as compound 9 (R₃═(C₁-C₁₂)-alkyl) and compound 10 (R₃═H), wherein Z is

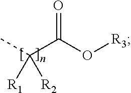

### B is

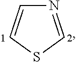

wherein 1 and 2 are the points of attachment of B to phenyl and to Z respectively; L=*NHC(O)NH, wherein * indicates the point of attachment of L to A; A, n, R₁ and R₂ are as defined in formula 1). Said process includes steps 1 to 8 as described below:

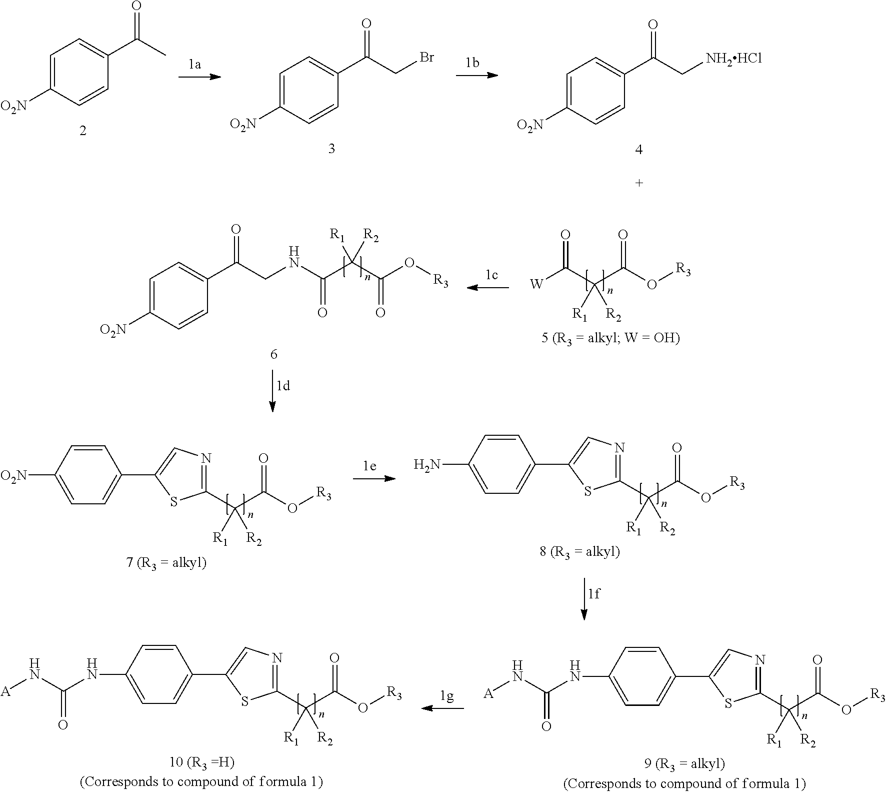

### Step 1

### Preparation of Compound of Formula 3:

Commercially available compound of formula 2 is subjected to bromination in presence of suitable catalyst such as anhydrous AlCl₃ in a suitable solvent such as dry ether at a temperature range of 0° C. to 35° C. for 4-8 h to yield compound of formula 3 (Reaction 1a).

### Step 2

### Preparation of Compound of Formula 4:

The compound of formula 3 is stirred with hexamethylene tetramine in a suitable solvent such as dichloromethane or chloroform at room temperature for 4-16 h, to yield the corresponding hexamine salt, which is hydrolysed by HCl in a suitable solvent such as ethanol or methanol to yield the compound of formula 4 (Reaction 1b).

### Step 3

### Preparation of Compound of Formula 6:

The compound of formula 5 is reacted with a reagent such as isobutylchloroformate in presence of a suitable base such as N-methylmorpholine in a solvent such as THF or DMF at a temperature range of −20° C. to −30° C. to form a carbonate, which is further reacted with the compound of formula 4 in presence of a suitable base such as triethylamine in a solvent such as THF or DMF at room temperature, to yield the compound of formula 6 (Reaction 1c).

The compound of formula 5 is prepared by the partial hydrolysis of the corresponding diester by using a reagent such as methanolic KOH. Alternatively, the compound of formula 5 is prepared by treatment of the corresponding anhydride with an inorganic acid such as concentrated H₂SO₄ in a solvent such as methanol.

### Step 4

### Preparation of Compound of Formula 7:

The compound of formula 6 is refluxed with a reagent such as Lawesson's reagent in a suitable solvent such as 1,4-dioxane or THF, at a temperature range of 60° C. to 110° C., to yield the compound of formula 7 (Reaction 1d).

### Step 5

### Preparation of Compound of Formula 8:

The compound of formula 7 is reduced with a suitable reducing agent such as Fe and NH₄Cl in a suitable solvent mixture of EtOH, THF and water at a temperature range of 70° C. to 80° C. for 2-6 h to yield compound of formula 8 (Reaction 1e).

### Step 6

### Preparation of Compound of Formula 9:

The compound of formula 8 is reacted with commercially available compound of formula 8 (i) in a suitable solvent such as THF or dichloromethane at room temperature for 2-16 h to yield the compound of formula 9 (Reaction 1f);

A-N═C═O 8 (i)

wherein A is as defined in formula 1.

Alternately, the compound of formula 8 is reacted with the compound of formula 8 (ii) in presence of a coupling agent such as carbonyl diimidazole in a suitable solvent such as THF at room temperature for about 24 h;

A-NH₂ 8 (ii)

wherein A is as defined in formula 1 to yield the compound of formula 9.

### Step 7

### Preparation of Compound of Formula 10:

The compound of formula 9 is hydrolysed using suitable reagent such as aqueous

LiOH in a suitable solvent such as THF or methanol or a mixture thereof, at room temperature for 2-16 h, to yield the compound of formula 10 (Reaction 1g).

### Step 8

The carboxylic acid (compound of formula 10) is optionally converted to its corresponding ester prodrugs by any suitable method well known in the art.

### Scheme 1B:

Scheme 1B depicts a process for the preparation of the compounds of formula 1 (referred in Scheme 1B as compound II (R₃═(C₁-C₁₂)-alkyl) and compound 12 (R₃═H), wherein Z is

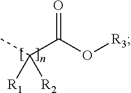

### B is

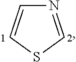

wherein 1 and 2 are the points of attachment of B to phenyl and to Z respectively; L=*NHC(S)NH, wherein * indicates the point of attachment of L to A; A, n, R₁ and R₂ are as defined in formula 1). Said process includes steps 1 to 3 as described below:

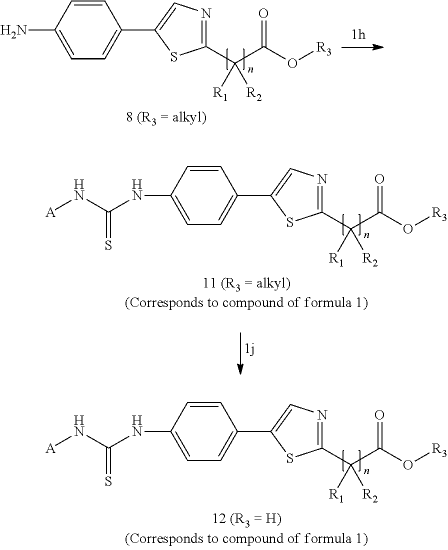

### Step 1

### Preparation of Compound of Formula 11:

The compound of formula 8 is reacted with compound of formula 8 (iii) in a suitable solvent such as THF or dichloromethane at room temperature for 2-16 h, to yield the compound of formula 11 (Reaction 1 h);

A-N═C═S 8 (iii)

wherein A is as defined in formula 1.

### Step 2

### Preparation of Compound of Formula 12:

The compound of formula 11 is hydrolysed using suitable reagent such as aqueous LiOH in a suitable solvent such as THF or methanol or a mixture thereof, at room temperature for 2-16 h at room temperature, to yield the compound of formula 12 (Reaction 1j).

### Step 3

The carboxylic acid (compound of formula 12) is optionally converted to its corresponding ester prodrugs by any suitable method well known in the art.

### Scheme 1C:

Scheme 1C depicts a process for the preparation of the compounds of formula 1 (referred in Scheme 1C as compound 13 (R₃═(C₁-C₁₂)-alkyl) and compound 14 (R₃═H), wherein Z is

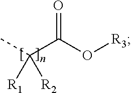

### B is

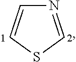

wherein 1 and 2 are the points of attachment of B to phenyl and to Z respectively; L=*C(O)NH, wherein * indicates the point of attachment of L to A; A, n, R₁ and R₂ are as defined in formula 1). Said process includes steps 1 to 3 as described below:

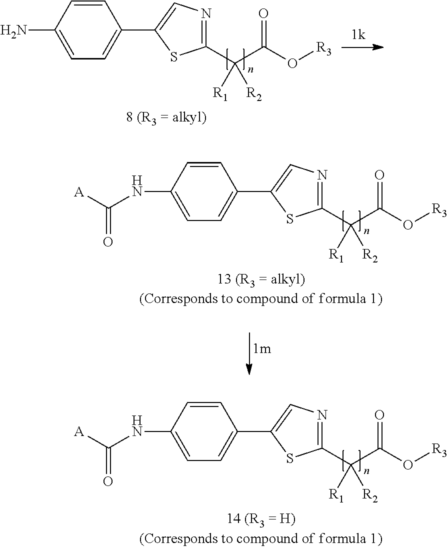

### Step 1

### Preparation of Compound of Formula 13:

The compound of formula 8 is reacted with commercially available compound of formula 8 (iv) in a suitable solvent such as dichloromethane or chloroform in a suitable base such as pyridine at room temperature for 1-2 h, to yield the compound of formula 13 (Reaction 1k);

A-C(O)—Cl 8 (iv)

wherein A is as defined in formula 1.

Alternately, the compound of formula 8 is reacted with commercially available compound of formula 8 (v) in a suitable solvent such as toluene and a coupling agent such as trimethylaluminium;

A-COOR₃ 8(v)

wherein A and R₃ are as defined in formula 1 to yield the compound of formula 13.

### Step 2

### Preparation of Compound of Formula 14:

The compound of formula 13 is hydrolysed using suitable reagent such as aqueous LiOH in a suitable solvent such as THF or methanol or a mixture thereof, at room temperature for 2-16 h at room temperature, to yield the compound of formula 14 (Reaction 1 m).

### Step 3

The carboxylic acid (compound of formula 14) is optionally converted to its corresponding ester prodrugs by any suitable method well known in the art.

### Scheme 10:

Scheme 1D depicts a process for the preparation of the compounds of formula 1 (referred in Scheme 1D as compound 15 (R₃═(C₁-C₁₂)-alkyl) and compound 16 (R₃═H), wherein Z is

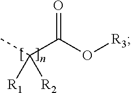

### B is

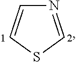

wherein 1 and 2 are the points of attachment of B to phenyl and to Z respectively; L=*SO₂NH, wherein * indicates the point of attachment of L to A; A, n, R₁ and R₂ are as defined in formula 1). Said process includes steps 1 to 3 as described below:

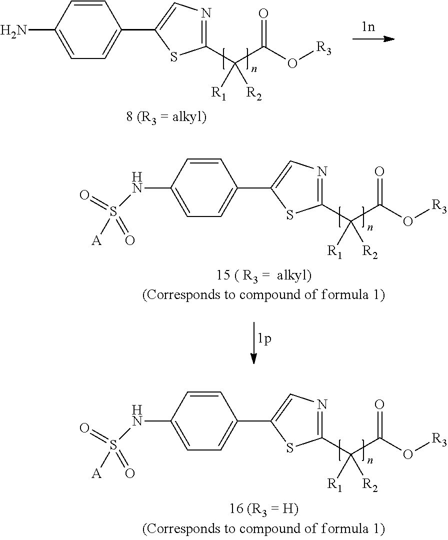

### Step 1

### Preparation of Compound of Formula 15:

The compound of formula 8 is reacted with compound of formula 8 (vi) in a suitable solvent such as dichloromethane or chloroform in a suitable base such as pyridine at room temperature for 1-2 h, to yield the compound of formula 15 (Reaction 1n);

A-SO₂—Cl 8 (vi)

wherein A is as defined in formula 1.

### Step 2

### Preparation of Compound of Formula 16:

The compound of formula 15 is hydrolysed using suitable reagent such as aqueous LiOH in a suitable solvent such as THF or methanol or a mixture thereof, at room temperature for 2-16 h at room temperature, to yield the compound of formula 16 (Reaction 1p).

### Step 3

The carboxylic acid (compound of formula 16) is optionally converted to its corresponding ester prodrugs by any suitable method well known in the art.

### Scheme 2A:

Scheme 2A depicts a process for the preparation of the compounds of formula 1 (referred in Scheme 2A as compound 19 (R₃═(C₁-C₁₂)-alkyl) and compound 20 (R₃═H), wherein Z is

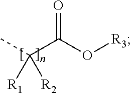

### B is

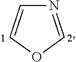

wherein 1 and 2 are the points of attachment of B to phenyl and to Z respectively; L=*NHC(O)NH, wherein * indicates the point of attachment of L to A; A, n, R₁ and R₂ are as defined in formula 1). Said process includes steps 1 to 5 as described below:

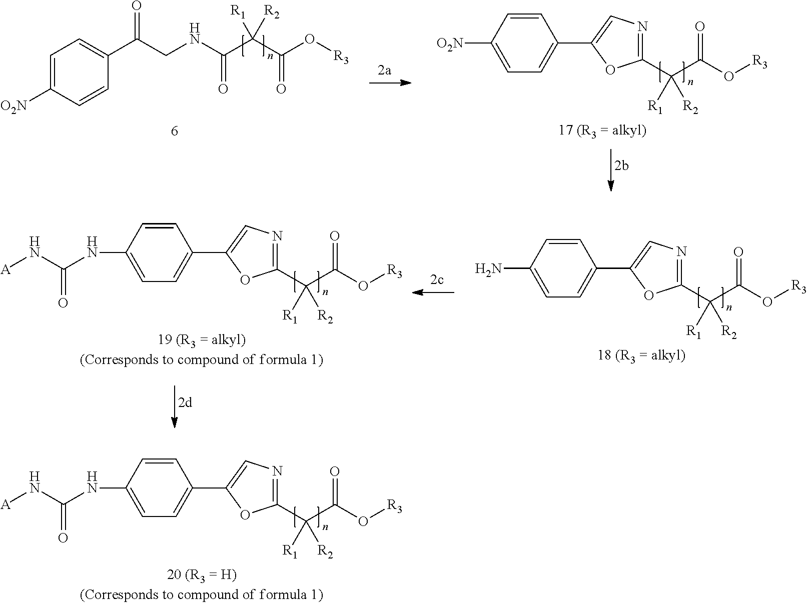

### Step 1

### Preparation of Compound of Formula 17:

The compound of formula 6 is refluxed with POCl₃, optionally in presence of solvent such as acetonitrile, at a temperature range of 80° C. to 110° C. for 2-3 h, to yield compound of formula 17 (Reaction 2a).

### Step 2

### Preparation of Compound of Formula 18:

The compound of formula 17 is reduced with a suitable reducing agent such as Fe and NH₄Cl in a suitable solvent mixture of EtOH, THF and water at a temperature range of 70° C. to 80° C. for 2-6 h, to yield compound of formula 18 (Reaction 2b).

### Step 3

### Preparation of Compound of Formula 19:

The compound of formula 18 is reacted with a compound of formula 8 (i) in a suitable solvent such as THF or dichloromethane at room temperature for 2-16 h, to yield the compound of formula 19 (Reaction 2c).

Alternately, the compound of formula 18 is reacted with the compound of formula 8 (ii) in presence of a coupling agent such as carbonyl diimidazole in a suitable solvent such as THF at room temperature for about 24 h to yield the compound of formula 19.

### Step 4

### Preparation of Compound of Formula 20:

The compound of formula 19 is hydrolysed using suitable reagent such as aqueous LiOH in a suitable solvent such as THF or methanol or a mixture thereof, at room temperature for 2-16 h at room temperature, to yield the compound of formula 20 (Reaction 2d).

### Step 5

The carboxylic acid (compound of formula 20) is optionally converted to its corresponding ester prodrugs by any suitable method well known in the art.

### Scheme 2B:

Scheme 2B depicts a process for the preparation of the compounds of formula 1 (referred in Scheme 2B as compound 21 (R₃=(C₁-C₁₂)-alkyl) and compound 22 (R₃═H), wherein Z is

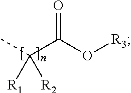

### B is

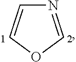

wherein 1 and 2 are the points of attachment of B to phenyl and to Z respectively; L=*NHC(S)NH, wherein * indicates the point of attachment of L to A; A, n, R₁ and R₂ are as defined in formula 1). Said process includes steps 1 to 3 as described below:

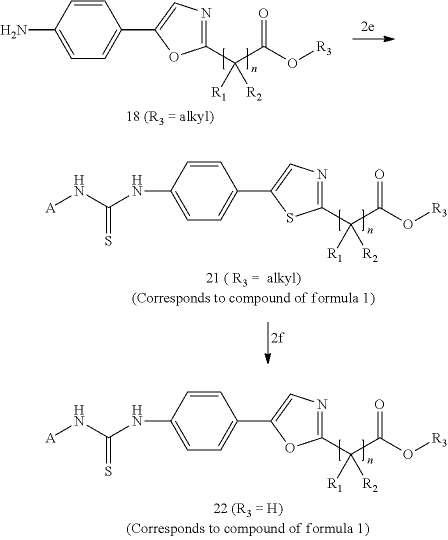

### Step 1

### Preparation of Compound of Formula 21:

The compound of formula 18 is reacted with a compound of formula 8 (iii) in a suitable solvent such as THF or dichloromethane at room temperature for 2-16 h, to yield the compound of formula 21 (Reaction 2e).

### Step 2

### Preparation of Compound of Formula 22:

The compound of formula 21 is hydrolysed using suitable reagent such as aqueous LiOH in a suitable solvent such as THF or methanol or a mixture thereof, at room temperature for 2-16 h at room temperature, to yield the compound of formula 22 (Reaction 2f).

### Step 3

The carboxylic acid (compound of formula 22) is optionally converted to its corresponding ester prodrugs by any suitable method well known in the art.

### Scheme 2C:

Scheme 2C depicts a process for the preparation of the compounds of formula 1 (referred in Scheme 2C as compound 23 (R₃═(C₁-C₁₂)-alkyl) and compound 24 (R₃═H), wherein Z is

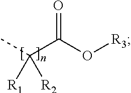

### B is

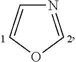

wherein 1 and 2 are the points of attachment of B to phenyl and to Z respectively; L=*CONH, wherein * indicates the point of attachment of L to A; A, n, R₁ and R₂ are as defined in formula 1). Said process includes steps 1 to 3 as described below:

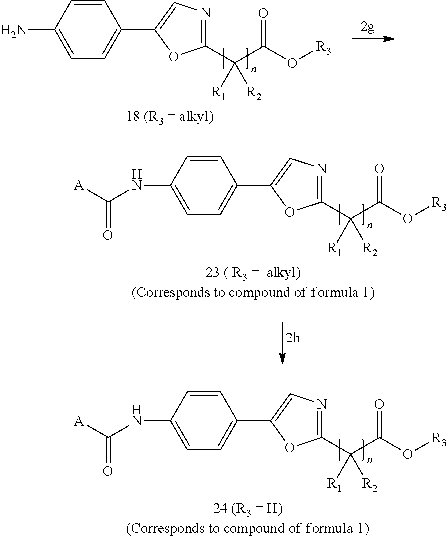

### Step 1

### Preparation of Compound of Formula 23:

The compound of formula 18 is reacted with a compound of formula 8 (iv) in a suitable solvent such as dichloromethane or chloroform in a suitable base such as pyridine at room temperature for 1-2 h, to yield the compound of formula 23 (Reaction 2g).

Alternately, the compound of formula 18 is reacted with the compound of formula 8 (v) in a suitable solvent such as toluene and a coupling agent such as trimethylaluminium to yield the compound of formula 23.

### Step 2

### Preparation of Compound of Formula 24:

The compound of formula 23 is hydrolysed using suitable reagent such as aqueous LiOH in a suitable solvent such as THF or methanol or a mixture thereof, at room temperature for 2-16 h at room temperature, to yield the compound of formula 24 (Reaction 2h).

### Step 3

The carboxylic acid (compound of formula 24) is optionally converted to its corresponding ester prodrugs by any suitable method well known in the art.

### Scheme 2D:

Scheme 2D depicts a process for the preparation of the compounds of formula 1 (referred in Scheme 2D as compound 25 (R₃═(C₁-C₁₂)-alkyl) and compound 26 (R₃═H), wherein Z is

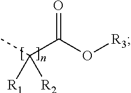

### B is

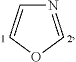

wherein 1 and 2 are the points of attachment of B to phenyl and to Z respectively; L=*SO₂NH, wherein * indicates the point of attachment of L to A; A, n, R₁ and R₂ are as defined in formula 1). Said process includes steps 1 to 3 as described below:

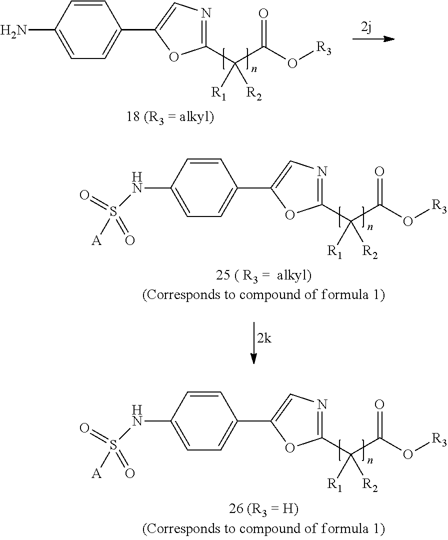

### Step 1

### Preparation of Compound of Formula 25:

The compound of formula 18 is reacted with a compound of formula 8 (vi) in a suitable solvent such as dichloromethane or chloroform in a suitable base such as pyridine at room temperature for 1-2 h, to yield the compound of formula 25 (Reaction 2j).

### Step 2

### Preparation of Compound of Formula 26:

The compound of formula 25 is hydrolysed using suitable reagent such as aqueous LiOH in a suitable solvent such as THF or methanol or a mixture thereof, at room temperature for 2-16 h at room temperature, to yield the compound of formula 26 (Reaction 2k).

### Step 3

The carboxylic acid (compound of formula 26) is optionally converted to its corresponding ester prodrugs by any suitable method well known in the art.

### Scheme 3A:

Scheme 3A depicts a process for the preparation of the compounds of formula 1 (referred in Scheme 3A as compound 30 (R₃═(C₁-C₁₂)-alkyl) and compound 31 (R₃═H), wherein Z is

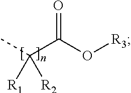

### B is

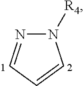

wherein 1 and 2 are the points of attachment of B to phenyl and to Z respectively; L=*NHC(O)NH, wherein * indicates the point of attachment of L to A; A, n, R₁, R₂ and R₄ are as defined in formula 1). Said process includes steps 1 to 6 as described below:

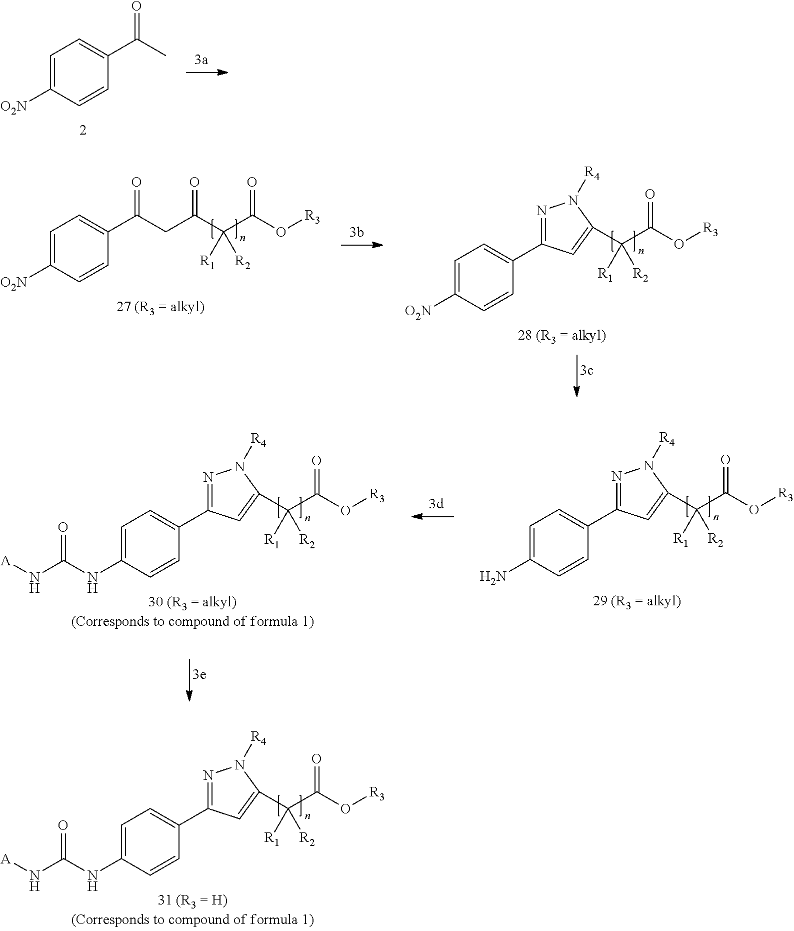

### Step 1

### Preparation of Compound of Formula 27:

The compound of formula 2 is reacted with the compound of formula 5 in a suitable solvent such as toluene, ethanol or THF at a temperature range of 60° C. to 120° C., optionally in presence of a suitable base such as sodium hydride, potassium carbonate or cesium carbonate, to yield the compound of formula 27 (Reaction 3a).

### Step 2

### Preparation of Compound of Formula 28:

The compound of formula 27 is refluxed with commercially available compound of formula 27 (i);

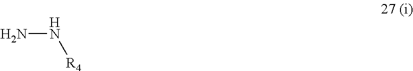

wherein R₄ is as defined in formula 1; in a suitable solvent such as ethanol or methanol at a suitable temperature of 60° C. to 85° C. to yield the compound of formula 28 (Reaction 3b).

### Step 3

### Preparation of Compound of Formula 29:

The compound of formula 28 is reduced with a suitable reducing agent such as Fe and NH₄Cl in a suitable solvent mixture of EtOH, THF and water at a temperature range of 70° C. to 80° C. for 2-6 h, to yield compound of formula 29 (Reaction 3c).

### Step 4

### Preparation of Compound of Formula 30:

The compound of formula 29 is reacted with a compound of formula 8 (i) in a suitable solvent such as THF or dichloromethane at room temperature for 2-16 h, to yield the compound of formula 30 (Reaction 3d).

Alternately, the compound of formula 29 is reacted with the compound of formula 8 (ii) in presence of a coupling agent such as carbonyl diimidazole in a suitable solvent such as THF at room temperature for about 24 h to yield the compound of formula 30.

### Step 5

### Preparation of Compound of Formula 31:

The compound of formula 30 is hydrolysed using suitable reagent such as aqueous LiOH in a suitable solvent such as THF or methanol or a mixture thereof, at room temperature for 2-16 h at room temperature, to yield the compound of formula 31 (Reaction 3e).

### Step 6

The carboxylic acid (compound of formula 31) is optionally converted to its corresponding ester prodrugs by any suitable method well known in the art.

### Scheme 3B:

Scheme 3B depicts a process for the preparation of the compounds of formula 1 (referred in Scheme 3B as compound 32 (R₃═(C₁-C₁₂)-alkyl) and compound 33 (R₃═H), wherein Z is

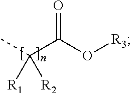

### B is

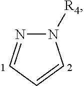

wherein 1 and 2 are the points of attachment of B to phenyl and to Z respectively; L=*NHC(S)NH, wherein * indicates the point of attachment of L to A; A, n, R₁, R₂ and R₄ are as defined in formula 1). Said process includes steps 1 to 3 as described below:

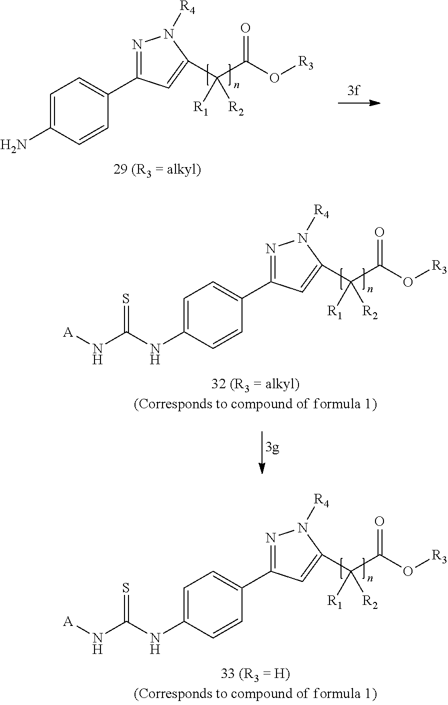

### Step 1

### Preparation of Compound of Formula 32:

The compound of formula 29 is reacted with a compound of formula 8 (iii) in a suitable solvent such as THF or dichloromethane at room temperature for 2-16 h, to yield the compound of formula 32 (Reaction 3f).

### Step 2

### Preparation of Compound of Formula 33:

The compound of formula 32 is hydrolysed using suitable reagent such as aqueous LiOH in a suitable solvent such as THF or methanol or a mixture thereof, at room temperature for 2-16 h at room temperature, to yield the compound of formula 33 (Reaction 3g).

### Step 3

The carboxylic acid (compound of formula 33) is optionally converted to its corresponding ester prodrugs by any suitable method well known in the art.

### Scheme 3C:

Scheme 3C depicts a process for the preparation of the compounds of formula 1 (referred in Scheme 3C as compound 34 (R₃═(C₁-C₁₂)-alkyl) and compound 35 (R₃═H), wherein Z is

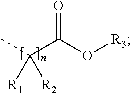

### B is

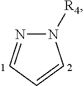

wherein 1 and 2 are the points of attachment of B to phenyl and to Z respectively; L=*CONH, wherein * indicates the point of attachment of L to A; A, n, R₁, R₂ and R₄ are as defined in formula 1). Said process includes steps 1 to 3 as described below:

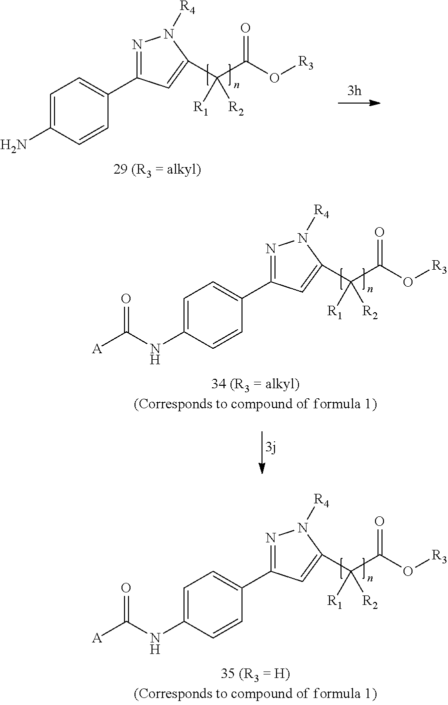

### Step 1

### Preparation of Compound of Formula 34:

The compound of formula 29 is reacted with a compound of formula 8 (iv) in a suitable solvent such as dichloromethane or chloroform in a suitable base such as pyridine at room temperature for 1-2 h, to yield the compound of formula 23 (Reaction 3h). Alternately, the compound of formula 29 is reacted with the compound of formula 8 (v) in a suitable solvent such as toluene and a coupling agent such as trimethylaluminium to yield the compound of formula 23.

### Step 2

### Preparation of Compound of Formula 35:

The compound of formula 34 is hydrolysed using suitable reagent such as aqueous LiOH in a suitable solvent such as THF or methanol or a mixture thereof, at room temperature for 2-16 h at room temperature, to yield the compound of formula 35 (Reaction 3j).

### Step 3

The carboxylic acid (compound of formula 35) is optionally converted to its corresponding ester prodrugs by any suitable method well known in the art.

### Scheme 3D:

Scheme 3D depicts a process for the preparation of the compounds of formula 1 (referred in Scheme 3D as compound 36 (R₃═(C₁-C₁₂)-alkyl) and compound 37 (R₃═H), wherein Z is

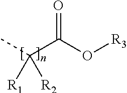

### B is

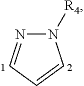

wherein 1 and 2 are the points of attachment of B to phenyl and to Z respectively; L=*SO₂NH, wherein * indicates the point of attachment of L to A; A, n, R₁, R₂ and R₄ are as defined in formula 1). Said process includes steps 1 to 3 as described below:

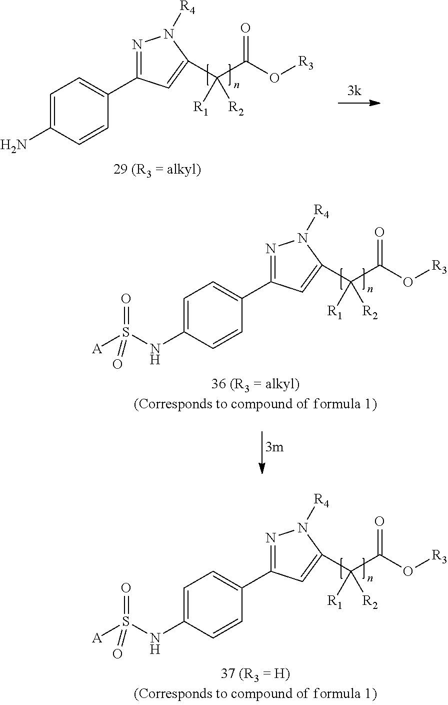

### Step 1

### Preparation of Compound of Formula 36:

The compound of formula 29 is reacted with a compound of formula 8 (vi) in a suitable solvent such as dichloromethane or chloroform in a suitable base such as pyridine at room temperature for 1-2 h, to yield the compound of formula 36 (Reaction 3k).

### Step 2

### Preparation of Compound of Formula 37:

The compound of formula 36 is hydrolysed using suitable reagent such as aqueous LiOH in a suitable solvent such as THF or methanol or a mixture thereof, at room temperature for 2-16 h at room temperature, to yield the compound of formula 37 (Reaction 3m).

### Step 3

The carboxylic acid (compound of formula 37) is optionally converted to its corresponding ester prodrugs by any suitable method well known in the art.

### Scheme 4A:

Scheme 4A depicts a process for the preparation of the compounds of formula 1 (referred in Scheme 4A as compound 44 (R₃═(C₁-C₁₂)-alkyl) and compound 45 (R₃═H), wherein Z is

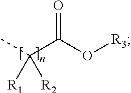

### B is

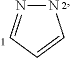

wherein 1 and 2 are the points of attachment of B to phenyl and to Z respectively; L=*NHC(O)NH, wherein * indicates the point of attachment of L to A; A, n, R₁ and R₂ are as defined in formula 1). Said process includes steps 1 to 8 as described below:

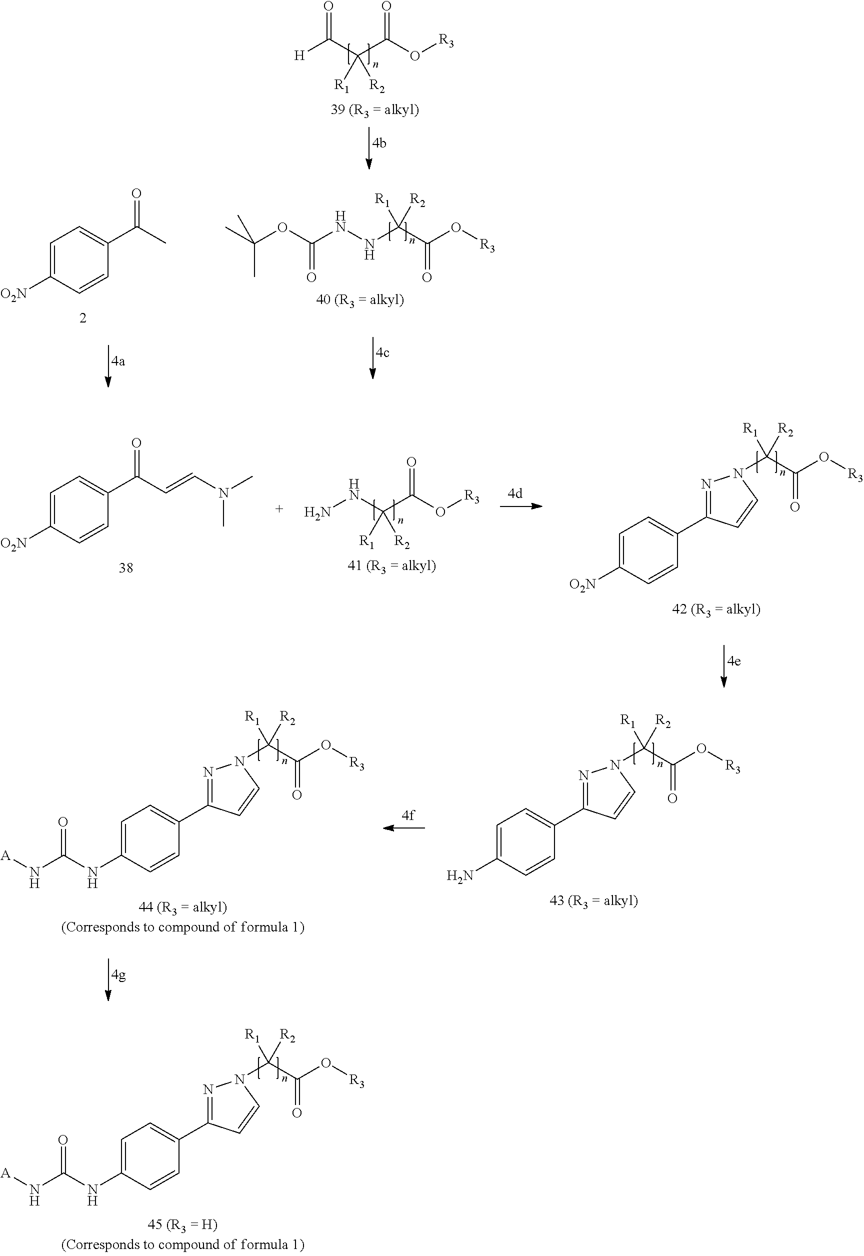

### Step 1

### Preparation of Compound of Formula 38:

The compound of formula 2 is reacted with commercially available compound of formula 2(i);

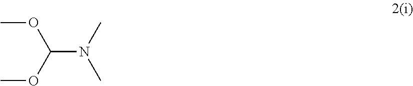

at a temperature range of 100-130° C. for about 17 h to yield the compound of formula 38 (Reaction 4a), according to the procedure disclosed in U.S. Pat. No. 4,699,915.

### Step 2

### Preparation of Compound of Formula 40:

Commercially available compound of formula 39 is treated with tert-butyl carbazate followed by reaction with sodium triacetoxy borohydride or borane-THF complex at a temperature range of 0° C. to 35° C. for about 7 h, to yield the compound of formula 40 (Reaction 4b), according to the procedure disclosed in EP2103603.

### Step 3

### Preparation of Compound of Formula 41:

The compound of formula 40 is treated with 4N HCl in dioxane at a temperature range of 25° C. to 50° C. for about 10 h, to yield the compound of formula 41 (Reaction 4c).

### Step 4

### Preparation of Compound of Formula 42:

The compound of formula 38 is reacted with the compound of formula 41 in a suitable solvent such as EtOH or methanol at a temperature range of 50-80° C. to yield the compound of formula 42 (Reaction 4d), according to the procedure disclosed in U.S. Pat. No. 4,699,915.

### Step 5

### Preparation of Compound of Formula 43:

The compound of formula 42 is reduced with a suitable reducing agent such as Fe and NH₄Cl in a suitable solvent mixture of EtOH, THF and water at a temperature range of 70° C. to 80° C. for 2-6 h, to yield the compound of formula 43 (Reaction 4e).

### Step 6

### Preparation of Compound of Formula 44:

The compound of formula 43 is reacted with a compound of formula 8 (i) in a suitable solvent such as THF or dichloromethane at room temperature for 2-16 h, to yield the compound of formula 44 (Reaction 4f).

Alternately, the compound of formula 43 is reacted with the compound of formula 8 (ii) in presence of a coupling agent such as carbonyl diimidazole in a suitable solvent such as THF at room temperature for about 24 h to yield the compound of formula 44.

### Step 7

### Preparation of Compound of Formula 45:

The compound of formula 44 is hydrolysed using suitable reagent such as aqueous LiOH in a suitable solvent such as THF or methanol or a mixture thereof, at room temperature for 2-16 h at room temperature, to yield the compound of formula 45 (Reaction 4g).

### Step 8

The carboxylic acid (compound of formula 45) is optionally converted to its corresponding ester prodrugs by any suitable method well known in the art.

### Scheme 4B:

Scheme 4B depicts a process for the preparation of the compounds of formula 1 (referred in Scheme 4B as compound 46 (R₃═(C₁-C₁₂)-alkyl) and compound 47 (R₃═H), wherein Z is

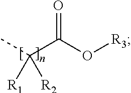

### B is

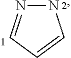

wherein 1 and 2 are the points of attachment of B to phenyl and to Z respectively; L=*NHC(S)NH, wherein * indicates the point of attachment of L to A; A, n, R₁ and R₂ are as defined in formula 1). Said process includes steps 1 to 3 as described below:

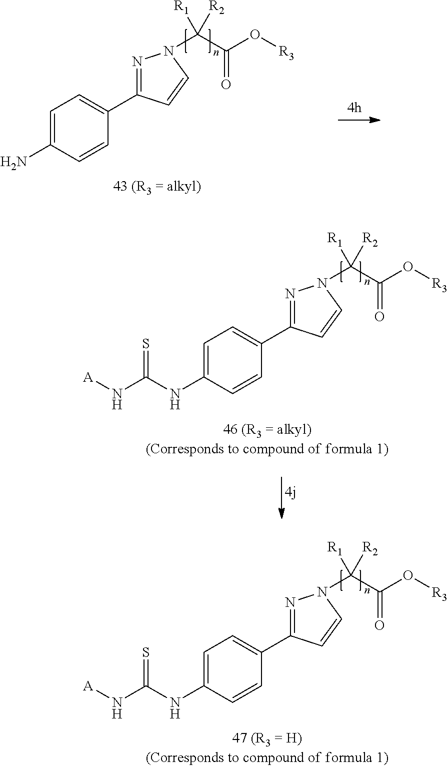

### Step 1

### Preparation of Compound of Formula 46:

The compound of formula 43 is reacted with a compound of formula 8 (iii) in a suitable solvent such as THF or dichloromethane at room temperature for 2-16 h, to yield the compound of formula 46 (Reaction 4h).

### Step 2

### Preparation of Compound of Formula 47:

The compound of formula 46 is hydrolysed using suitable reagent such as aqueous

LiOH in a suitable solvent such as THF or methanol or a mixture thereof, at room temperature for 2-16 h at room temperature, to yield the compound of formula 47 (Reaction 4j).

### Step 3

The carboxylic acid (compound of formula 47) is optionally converted to its corresponding ester prodrugs by any suitable method well known in the art.

### Scheme 4C:

Scheme 4C depicts a process for the preparation of the compounds of formula 1 (referred in Scheme 4C as compound 48 (R₃═(C₁-C₁₂)-alkyl) and compound 49 (R₃═H), wherein Z is

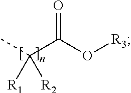

### B is

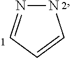

wherein 1 and 2 are the points of attachment of B to phenyl and to Z respectively; L=*CONH, wherein * indicates the point of attachment of L to A; A, n, R₁, R₂ and R₃ are as defined in formula 1). Said process includes steps 1 to 3 as described below:

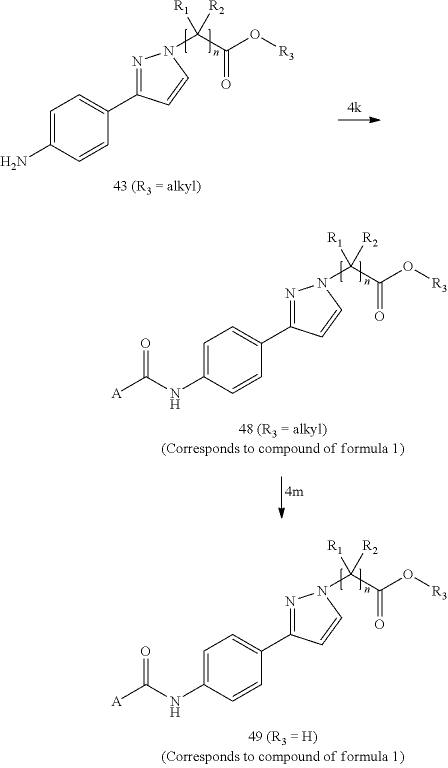

### Step 1

### Preparation of Compound of Formula 48:

The compound of formula 43 is reacted with a compound of formula 8 (iv) in a suitable solvent such as dichloromethane or chloroform in a suitable base such as pyridine at room temperature for 1-2 h, to yield the compound of formula 48 (Reaction 4k).

Alternately, the compound of formula 43 is reacted with the compound of formula 8 (v) in a suitable solvent such as toluene and a coupling agent such as trimethylaluminium to yield the compound of formula 48.

### Step 2

### Preparation of Compound of Formula 49:

The compound of formula 48 is hydrolysed using suitable reagent such as aqueous LiOH in a suitable solvent such as THF or methanol or a mixture thereof, at room temperature for 2-16 h at room temperature, to yield the compound of formula 49 (Reaction 4m).

### Step 3

The carboxylic acid (compound of formula 49) is optionally converted to its corresponding ester prodrugs by any suitable method well known in the art.

### Scheme 4D:

Scheme 4D depicts a process for the preparation of the compounds of formula 1 (referred in Scheme 4D as compound 50 (R₃═(C₁-C₁₂)-alkyl) and compound 51 (R₃═H), wherein Z is

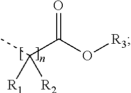

### B is

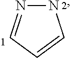

wherein 1 and 2 are the points of attachment of B to phenyl and to Z respectively; L=*SO₂NH, wherein * indicates the point of attachment of L to A; A, n, R₁ and R₂ are as defined in formula 1). Said process includes steps 1 to 3 as described below:

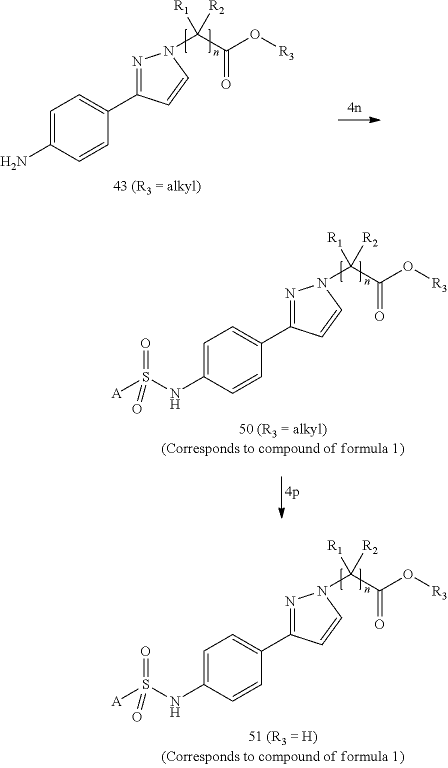

### Step 1

### Preparation of Compound of Formula 50:

The compound of formula 43 is reacted with a compound of formula 8 (vi) in a in a suitable solvent such as dichloromethane or chloroform in a suitable base such as pyridine at room temperature, to yield the compound of formula 50 (Reaction 4n).

### Step 2

### Preparation of Compound of Formula 51:

The compound of formula 50 is hydrolysed using suitable reagent such as aqueous

LiOH in a suitable solvent such as THF or methanol or a mixture thereof, at room temperature for 2-16 h at room temperature, to yield the compound of formula 51 (Reaction 4p).

### Step 3

The carboxylic acid (compound of formula 51) is optionally converted to its corresponding ester prodrugs by any suitable method well known in the art.

### Scheme 5A:

Scheme 5A depicts a process for the preparation of the compounds of formula 1 (referred in Scheme 5A as compound 57 (R₃═(C₁-C₁₂)-alkyl) and compound 58 (R₃═H), wherein Z is

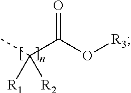

### B is

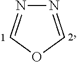

wherein 1 and 2 are the points of attachment of B to phenyl and to Z respectively; L=*NHC(O)NH, wherein * indicates the point of attachment of L to A; A, n, R₁, R₂ and R₃ are as defined in formula 1). Said process includes steps 1 to 7 as described below:

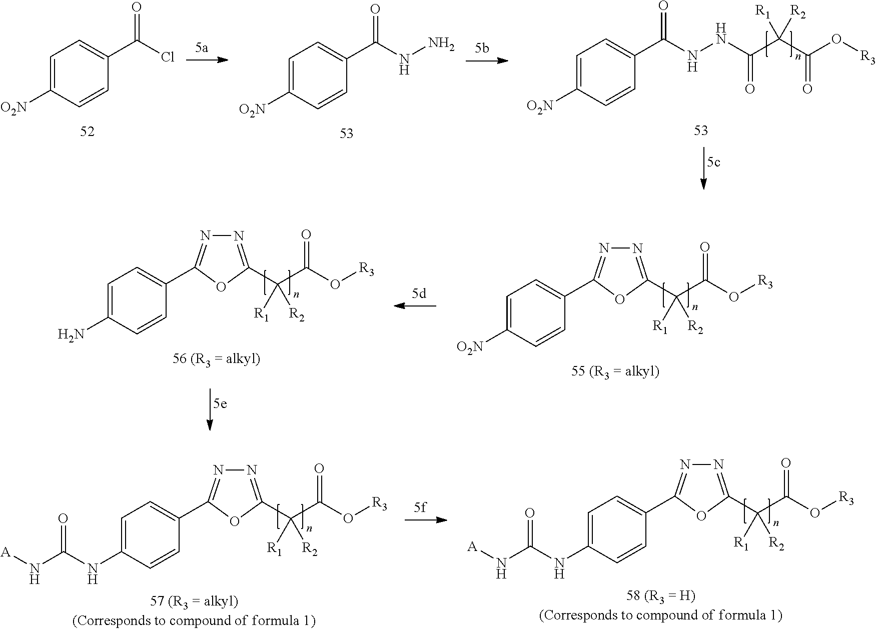

### Step 1

### Preparation of Compound of Formula 53:

Commercially available compound of formula 52 is refluxed with hydrazine in a suitable solvent such as methanol or ethanol for about 6 h, at a temperature range of 60° C. to 80° C., to yield the compound of formula 53 (Reaction 5a), according to the procedure described in Journal of Medicinal Chemistry, 2004, 47, 6764.

### Step 2

### Preparation of Compound of Formula 54:

The compound of formula 53 is reacted with the compound of formula 5 in a suitable solvent such as dichoromethane in presence of a suitable base such as triethylamine at room temperature for 10 to 18 h, to yield the compound of formula 54 (Reaction 5b).

### Step 3

### Preparation of Compound of Formula 55:

The compound of formula 54 is refluxed with POCl₃, optionally in presence of solvent such as acetonitrile, at a temperature range of 80° C. to 110° C. for 2-3 h, to obtain the compound of formula 55 (Reaction 5c).

### Step 4

### Preparation of Compound of Formula 56:

The compound of formula 55 is reduced with a suitable reducing agent such as Fe and NH₄Cl in a suitable solvent mixture of EtOH, THF and water at a temperature range of 70° C. to 80° C. for 2-6 h, to yield compound of formula 56 (Reaction 5d).

### Step 5

### Preparation of Compound of Formula 57:

The compound of formula 56 is reacted with a compound of formula 8 (i) in a suitable solvent such as THF or dichloromethane at room temperature for 2-16 h, to yield the compound of formula 57 (Reaction 5e).

Alternately, the compound of formula 8 is reacted with the compound of formula 8 (ii) in presence of a coupling agent such as carbonyl diimidazole in a suitable solvent such as THF at room temperature for about 24 h.

### Step 6

### Preparation of Compound of Formula 58:

The compound of formula 57 is hydrolysed using suitable reagent such as aqueous LiOH in a suitable solvent such as THF or methanol or a mixture thereof, at room temperature for 2-16 h at room temperature, to yield the compound of formula 58 (Reaction 5f).

### Step 7

The carboxylic acid (compound of formula 58) is optionally converted to its corresponding ester prodrugs by any suitable method well known in the art.

### Scheme 5B:

Scheme 5B depicts a process for the preparation of the compounds of formula 1 (referred in Scheme 5B as compound 59 (R₃=(C₁-C₁₂)-alkyl) and compound 60 (R₃═H), wherein Z is

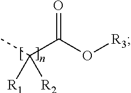

### B is

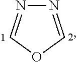

wherein 1 and 2 are the points of attachment of B to phenyl and to Z respectively; L=*NHC(S)NH, wherein * indicates the point of attachment of L to A; A, n, R₁ and R₂ are as defined in formula 1). Said process includes steps 1 to 3 as described below:

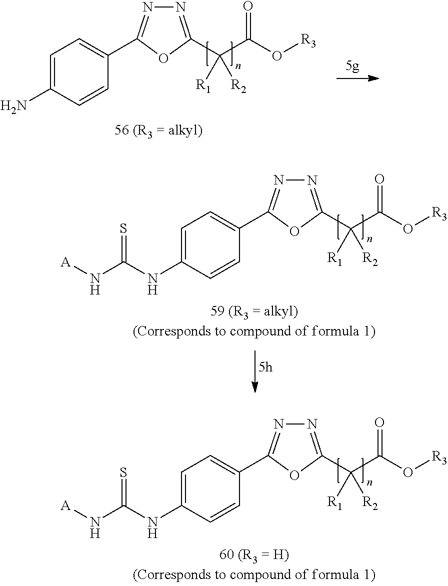

### Step 1

### Preparation of Compound of Formula 59:

The compound of formula 56 is reacted with a compound of formula 8 (iii) in a suitable solvent such as THF or dichloromethane at room temperature for 2-16 h, to yield the compound of formula 59 (Reaction 5g).

### Step 2

### Preparation of Compound of Formula 60:

The compound of formula 59 is hydrolysed using suitable reagent such as aqueous

LiOH in a suitable solvent such as THF or methanol or a mixture thereof, at room temperature for 2-16 h at room temperature, to yield the compound of formula 60 (Reaction 5h).

### Step 3

The carboxylic acid (compound of formula 60) is optionally converted to its corresponding ester prodrugs by any suitable method well known in the art.

### Scheme 5C:

Scheme 5C depicts a process for the preparation of the compounds of formula 1 (referred in Scheme 5C as compound 61 (R₃═(C₁-C₁₂)-alkyl) and compound 62 (R₃═H), wherein Z is

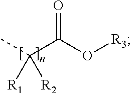

### B is

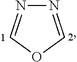

wherein 1 and 2 are the points of attachment of B to phenyl and to Z respectively; L=*CONH, wherein * indicates the point of attachment of L to A; A, n, R₁ and R₂ are as defined in formula 1). Said process includes steps 1 to 3 as described below:

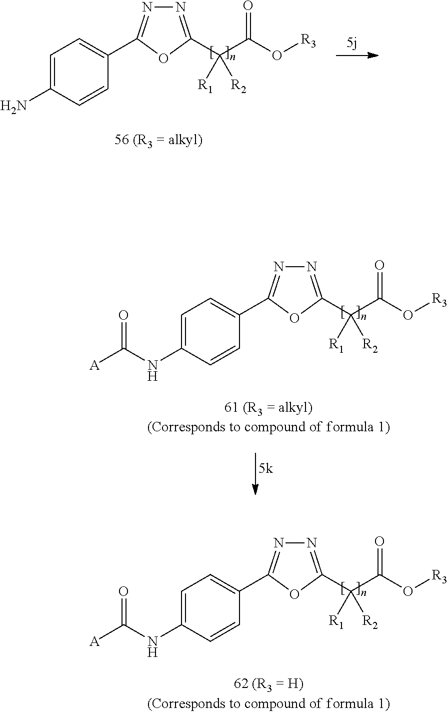

### Step 1

### Preparation of Compound of Formula 61:

The compound of formula 56 is reacted with a compound of formula 8 (iv) in a suitable solvent such as dichloromethane or chloroform in a suitable base such as pyridine at room temperature for 1-2 h, to yield the compound of formula 61 (Reaction 5j).

Alternately, the compound of formula 56 is reacted with the compound of formula 8 (v) in a suitable solvent such as toluene and a coupling agent such as trimethylaluminium to yield the compound of formula 61.

### Step 2

### Preparation of Compound of Formula 62:

The compound of formula 61 is hydrolysed using suitable reagent such as aqueous LiOH in a suitable solvent such as THF or methanol or a mixture thereof, at room temperature for 2-16 h at room temperature, to yield the compound of formula 62 (Reaction 5k).

### Step 3

The carboxylic acid (compound of formula 62) is optionally converted to its corresponding ester prodrugs by any suitable method well known in the art.

### Scheme 5D:

Scheme 5D depicts a process for the preparation of the compounds of formula 1 (referred in Scheme 5D as compound 63 (R₃═(C₁-C₁₂)-alkyl) and compound 64 (R₃═H), wherein Z is

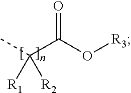

### B is

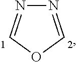

wherein 1 and 2 are the points of attachment of B to phenyl and to Z respectively; L=*SO₂NH, wherein * indicates the point of attachment of L to A; A, n, R₁ and R₂ are as defined in formula 1). Said process includes steps 1 to 3 as described below:

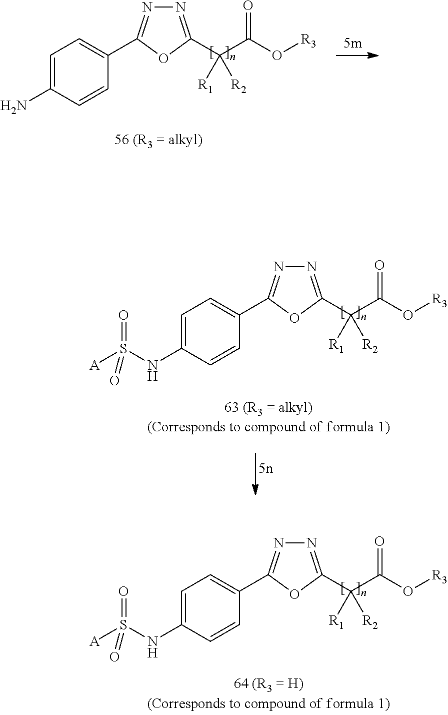

### Step 1

### Preparation of Compound of Formula 63:

The compound of formula 56 is reacted with a compound of formula 8 (vi) in a suitable solvent such as dichloromethane or chloroform in a suitable base such as pyridine at room temperature for 1-2 h, to yield the compound of formula 63 (Reaction 5m).

### Step 2

### Preparation of Compound of Formula 64:

The compound of formula 63 is hydrolysed using suitable reagent such as aqueous LiOH in a suitable solvent such as THF or methanol or a mixture thereof, at room temperature for 2-16 h at room temperature, to yield the compound of formula 64 (Reaction 5n).

### Step 3

The carboxylic acid (compound of formula 64) is optionally converted to its corresponding ester prodrugs by any suitable method well known in the art.

### Scheme 6A:

Scheme 6A depicts a process for the preparation of the compounds of formula 1 (referred in Scheme 6A as compound 67 (R₃═(C₁-C₁₂)-alkyl) and compound 68 (R₃═H), wherein Z is

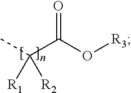

### B is

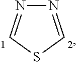

wherein 1 and 2 are the points of attachment of B to phenyl and to Z respectively; L=*NHC(O)NH, wherein * indicates the point of attachment of L to A; A, n, R₁ and R₂ are as defined in formula 1). Said process includes steps 1 to 5 as described below:

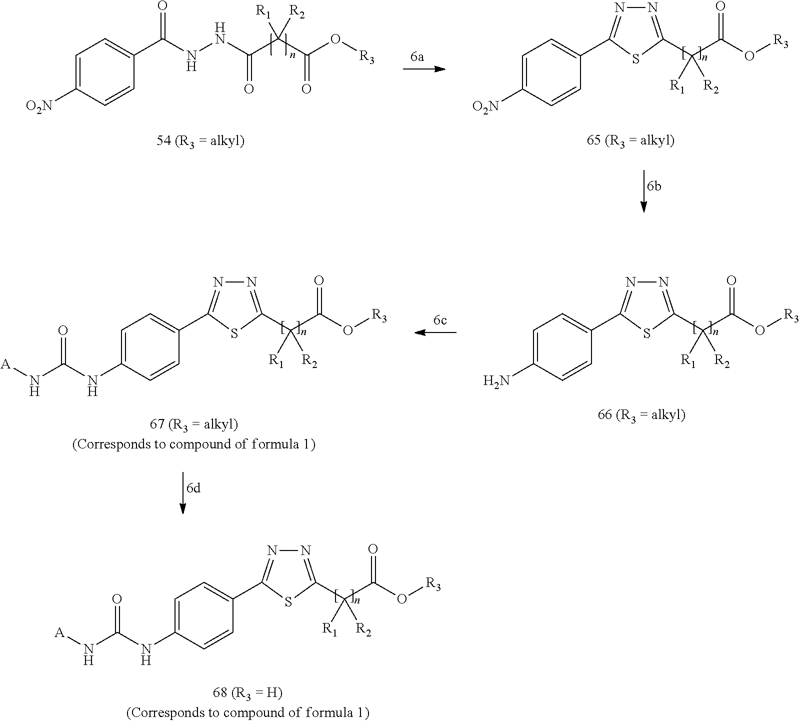

### Step 1

### Preparation of Compound of Formula 65:

The compound of formula 54 is refluxed with Lawesson's reagent in a suitable solvent such as 1,4-dioxane or THF, at a temperature range of 80° C. to 110° C., to yield the compound of formula 65 (Reaction 6a).

### Step 2

### Preparation of Compound of Formula 66:

The compound of formula 65 is reduced with a suitable reducing agent such as Fe and NH₄Cl in a suitable solvent mixture of EtOH, THF and water at a temperature range of 70° C. to 80° C. for 2-6 h, to yield compound of formula 66 (Reaction 6b).

### Step 3

### Preparation of Compound of Formula 67:

The compound of formula 66 is reacted with a compound of formula 8 (i) in a suitable solvent such as THF or dichloromethane at room temperature for 2-16 h, to yield the compound of formula 67 (Reaction 6c).

Alternately, the compound of formula 66 is reacted with commercially available compound of formula 8 (ii) in presence of a coupling agent such as carbonyl diimidazole in a suitable solvent such as THF at room temperature for about 24 h to yield the compound of formula 67.

### Step 4

### Preparation of Compound of Formula 68:

The compound of formula 67 is hydrolysed using suitable reagent such as aqueous LiOH in a suitable solvent such as THF or methanol or a mixture thereof, at room temperature for 2-16 h at room temperature, to yield the compound of formula 68 (Reaction 6d).

### Step 5

The carboxylic acid (compound of formula 68) is optionally converted to its corresponding ester prodrugs by any suitable method well known in the art.

### Scheme 6B:

Scheme 6B depicts a process for the preparation of the compounds of formula 1 (referred in Scheme 6B as compound 69 (R₃=(C₁-C₁₂)-alkyl) and compound 70 (R₃═H), wherein Z is

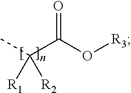

### B is

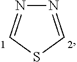

wherein 1 and 2 are the points of attachment of B to phenyl and to Z respectively; L=*NHC(S)NH, wherein * indicates the point of attachment of L to A; A, n, R₁ and R₂ are as defined in formula 1). Said process includes steps 1 to 3 as described below:

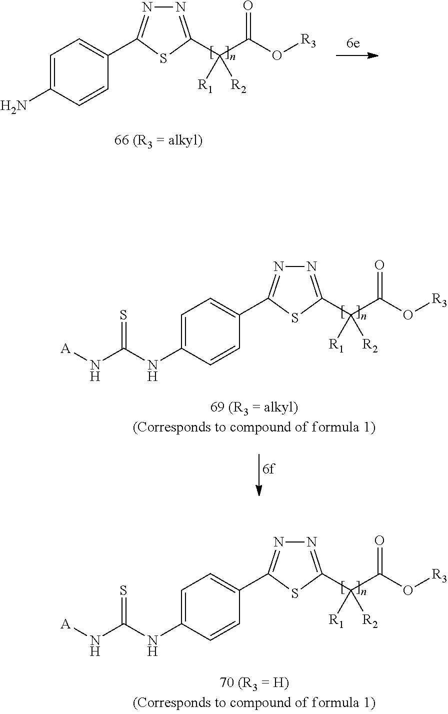

### Step 1

### Preparation of Compound of Formula 69:

The compound of formula 66 is reacted with a compound of formula 8 (iii) in a suitable solvent such as THF or dichloromethane at room temperature for 2-16 h, to yield the compound of formula 69 (Reaction 6e).

### Step 2

### Preparation of Compound of Formula 70:

The compound of formula 69 is hydrolysed using suitable reagent such as aqueous LiOH in a suitable solvent such as THF or methanol or a mixture thereof, at room temperature for 2-16 h at room temperature, to yield the compound of formula 70 (Reaction 6f).

### Step 3

The carboxylic acid (compound of formula 70) is optionally converted to its corresponding ester prodrugs by any suitable method well known in the art.

### Scheme 6C:

Scheme 6C depicts a process for the preparation of the compounds of formula 1 (referred in Scheme 6C as compound 71 (R₃═(C₁-C₁₂)-alkyl) and compound 72 (R₃═H), wherein Z is

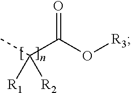

### B is

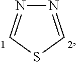

wherein 1 and 2 are the points of attachment of B to phenyl and to Z respectively; L=*CONH, wherein * indicates the point of attachment of L to A; A, n, R₁ and R₂ are as defined in formula 1). Said process includes steps 1 to 3 as described below:

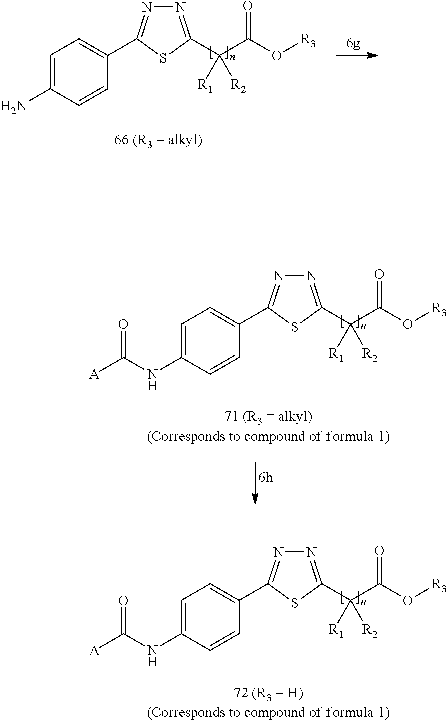

### Step 1

### Preparation of Compound of Formula 71:

The compound of formula 66 is reacted with a compound of formula 8 (iv) in a suitable solvent such as dichloromethane or chloroform in a suitable base such as pyridine at room temperature for 1-2 h, to yield the compound of formula 71 (Reaction 6g). Alternately, the compound of formula 66 is reacted with the compound of formula 8 (v) in a suitable solvent such as toluene and a coupling agent such as trimethylaluminium to yield the compound of formula 71.

### Step 2

### Preparation of Compound of Formula 72:

The compound of formula 71 is hydrolysed using suitable reagent such as aqueous LiOH in a suitable solvent such as THF or methanol or a mixture thereof, at room temperature for 2-16 h at room temperature, to yield the compound of formula 72 (Reaction 6h).

### Step 3

The carboxylic acid (compound of formula 72) is optionally converted to its corresponding ester prodrugs by any suitable method well known in the art.

### Scheme 6D:

Scheme 6D depicts a process for the preparation of the compounds of formula 1 (referred in Scheme 6D as compound 73 (R₃═(C₁-C₁₂)-alkyl) and compound 74 (R₃═H), wherein Z is

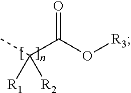

### B is

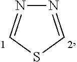

wherein 1 and 2 are the points of attachment of B to phenyl and to Z respectively; L=*SO₂NH, wherein * indicates the point of attachment of L to A; A, n, R₁ and R₂ are as defined in formula 1). Said process includes steps 1 to 3 as described below:

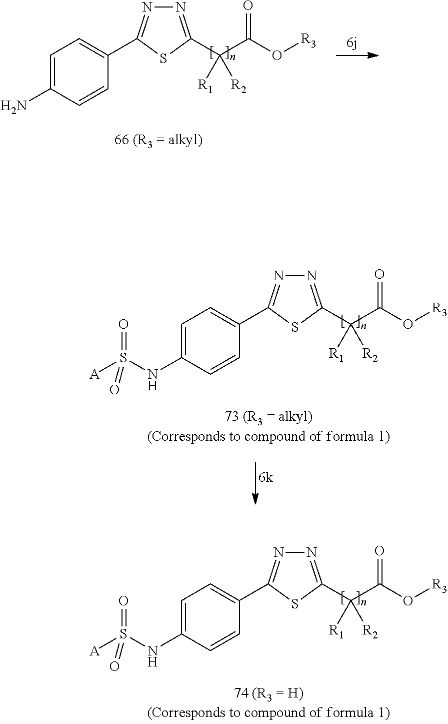

### Step 1

### Preparation of Compound of Formula 73:

The compound of formula 66 is reacted with a compound of formula 8 (vi) in a suitable solvent such as dichloromethane or chloroform in a suitable base such as pyridine at room temperature for 1-2 h, to yield the compound of formula 73 (Reaction 6j).

### Step 2

### Preparation of Compound of Formula 74:

The compound of formula 73 is hydrolysed using suitable reagent such as aqueous LiOH in a suitable solvent such as THF or methanol or a mixture thereof, at room temperature for 2-16 h at room temperature, to yield the compound of formula 74 (Reaction 6k).

### Step 3

The carboxylic acid (compound of formula 74) is optionally converted to its corresponding ester prodrugs by any suitable method well known in the art.

### Scheme 7A:

Scheme 7A depicts a process for the preparation of the compounds of formula 1 (referred in Scheme 7A as compound 79 (R₃═(C₁-C₁₂)-alkyl) and compound 80 (R₃═H), wherein Z is

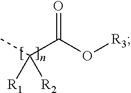

### B is

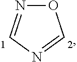

wherein 1 and 2 are the points of attachment of B to phenyl and to Z respectively; L=*NHC(O)NH, wherein * indicates the point of attachment of L to A; A, n, R₁ and R₂ are as defined in formula 1). Said process includes steps 1 to 6 as described below:

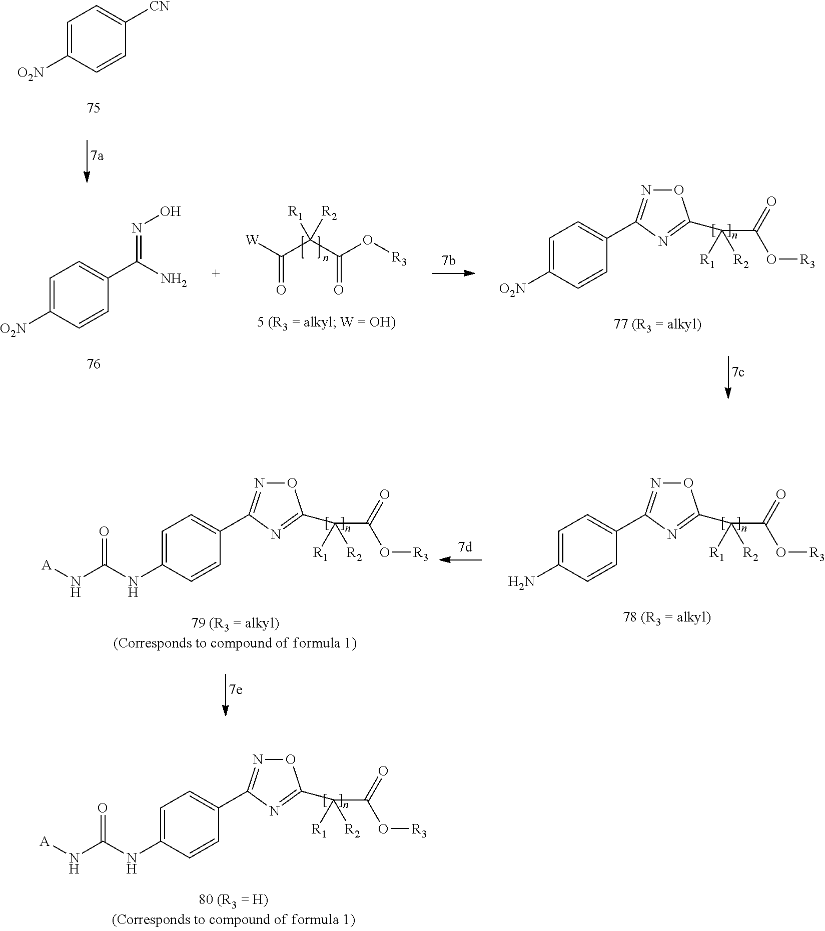

### Step 1

### Preparation of Compound of Formula 76:

Commercially available compound of formula 75 is reacted with hydroxylamine hydrochloride in presence of a suitable base such as K₂CO₃ in a suitable solvent such as MeOH or EtOH at a temperature range of 50° C. to 80° C. for 4-10 h, to yield the compound of formula 76 (Reaction 7a).

### Step 2

### Preparation of Compound of Formula 77:

The compound of formula 76 is reacted with the compound of formula 5 in a suitable solvent such as dichloromethane or chloroform in presence of a coupling reagent such as carbonylimidazole at room temperature for 8-10 h, followed by cyclisation by refluxing in a suitable solvent such as toluene at a temperature range of 100° C. to 130° C. for about 18 h, to yield the compound of formula 77 (Reaction 7b), according to the procedure as described in US2009/93516.

### Step 3

### Preparation of Compound of Formula 78:

The compound of formula 77 is reduced with a suitable reducing agent such as Fe and NH₄Cl in a suitable solvent mixture of EtOH, THF and water at a temperature range of 70° C. to 80° C. for 2-6 h, to yield compound of formula 78 (Reaction 7c).

### Step 4

### Preparation of Compound of Formula 79:

The compound of formula 78 is reacted with a compound of formula 8 (i) in a suitable solvent such as THF or dichloromethane at room temperature for 2-16 h, to yield the compound of formula 79 (Reaction 7d).

Alternately, the compound of formula 78 is reacted with the compound of formula 8 (ii) in presence of a coupling agent such as carbonyl diimidazole in a suitable solvent such as THF at room temperature for about 24 h to yield the compound of formula 79.

### Step 5

### Preparation of Compound of Formula 80:

The compound of formula 79 is hydrolysed using suitable reagent such as aqueous LiOH in a suitable solvent such as THF or methanol or a mixture thereof, at room temperature for 2-16 h at room temperature, to yield the compound of formula 80 (Reaction 7e).

### Step 6

The carboxylic acid (compound of formula 80) is optionally converted to its corresponding ester prodrugs by any suitable method well known in the art.

### Scheme 7B:

Scheme 7B depicts a process for the preparation of the compounds of formula 1 (referred in Scheme 7B as compound 81 (R₃═(C₁-C₁₂)-alkyl) and compound 82 (R₃═H), wherein Z is

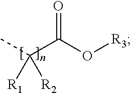

### B is

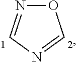

wherein 1 and 2 are the points of attachment of B to phenyl and to Z respectively; L=*NHC(S)NH, wherein * indicates the point of attachment of L to A; A, n, R₁ and R₂ are as defined in formula 1). Said process includes steps 1 to 3 as described below:

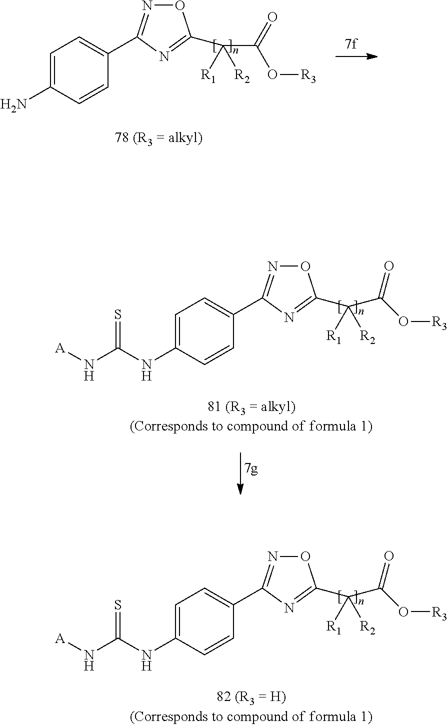

### Step 1

### Preparation of Compound of Formula 81:

The compound of formula 78 is reacted with a compound of formula 8 (iii) in a suitable solvent such as THF or dichloromethane at room temperature for 2-16 h, to yield the compound of formula 81 (Reaction 7f).

### Step 2

### Preparation of Compound of Formula 82:

The compound of formula 81 is hydrolysed using suitable reagent such as aqueous LiOH in a suitable solvent such as THF or methanol or a mixture thereof, at room temperature for 2-16 h at room temperature, to yield the compound of formula 82 (Reaction 7g).

### Step 3

The carboxylic acid (compound of formula 82) is optionally converted to its corresponding ester prodrugs by any suitable method well known in the art.

### Scheme 7C:

Scheme 7C depicts a process for the preparation of the compounds of formula 1 (referred in Scheme 7C as compound 83 (R₃═(C₁-C₁₂)-alkyl) and compound 84 (R₃═H), wherein Z is

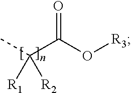

### B is

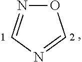

wherein 1 and 2 are the points of attachment of B to phenyl and to Z respectively; L=*CONH, wherein * indicates the point of attachment of L to A; A, n, R₁ and R₂ are as defined in formula 1). Said process includes steps 1 to 3 as described below:

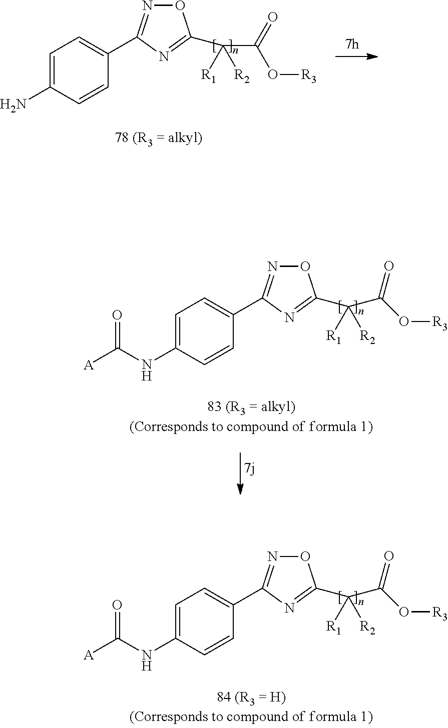

### Step 1

### Preparation of Compound of Formula 83:

The compound of formula 78 is reacted with a compound of formula 8 (iv) in a suitable solvent such as dichloromethane or chloroform in a suitable base such as pyridine at room temperature for 1-2 h, to yield the compound of formula 83 (Reaction 7h).

Alternately, the compound of formula 78 is reacted with the compound of formula 8 (v) in a suitable solvent such as toluene and a coupling agent such as trimethylaluminium to yield the compound of formula 83.

### Step 2

### Preparation of Compound of Formula 84:

The compound of formula 83 is hydrolysed using suitable reagent such as aqueous LiOH in a suitable solvent such as THF or methanol or a mixture thereof, at room temperature for 2-16 h at room temperature, to yield the compound of formula 84 (Reaction 7j).

### Step 3

The carboxylic acid (compound of formula 84) is optionally converted to its corresponding ester prodrugs by any suitable method well known in the art.

### Scheme 7D:

Scheme 7D depicts a process for the preparation of the compounds of formula 1 (referred in Scheme 7D as compound 85 (R₃═(C₁-C₁₂)-alkyl) and compound 86 (R₃═H), wherein Z is

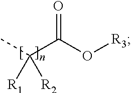

### B is

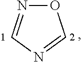

wherein 1 and 2 are the points of attachment of B to phenyl and to Z respectively; L=*SO₂NH, wherein * indicates the point of attachment of L to A; A, n, R₁ and R₂ are as defined in formula 1). Said process includes steps 1 to 3 as described below:

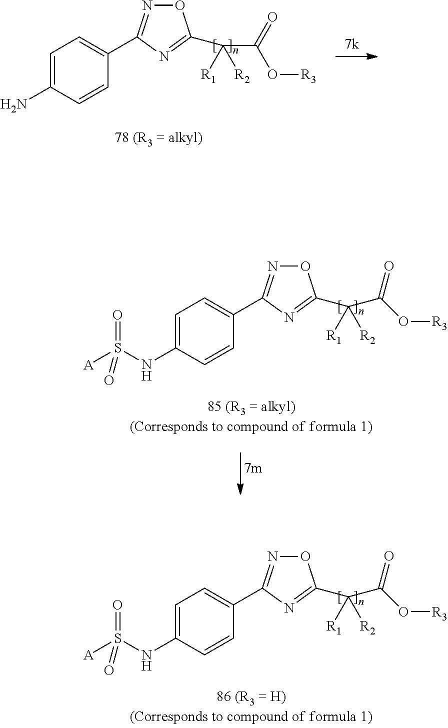

### Step 1

### Preparation of Compound of Formula 85:

The compound of formula 78 is reacted with a compound of formula 8 (vi) in a suitable solvent such as dichloromethane or chloroform in a suitable base such as pyridine at room temperature for 1-2 h, to yield the compound of formula 85 (Reaction 7k).

### Step 2

### Preparation of Compound of Formula 86:

The compound of formula 85 is hydrolysed using suitable reagent such as aqueous LiOH in a suitable solvent such as THF or methanol or a mixture thereof, at room temperature for 2-16 h at room temperature, to yield the compound of formula 86 (Reaction 7m).

### Step 3

The carboxylic acid (compound of formula 86) is optionally converted to its corresponding ester prodrugs by any suitable method well known in the art.

### Scheme 8A:

Scheme 8A depicts a process for the preparation of the compounds of formula 1 (referred in Scheme 8A as compound 91 (R₃═(C₁-C₁₂)-alkyl) and compound 92 (R₃═H), wherein Z is

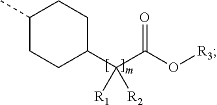

### B is

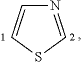

wherein 1 and 2 are the points of attachment of B to phenyl and to Z respectively; L=*NHC(O)NH, wherein * indicates the point of attachment of L to A; A, n, R₁ and R₂ are as defined in formula 1). Said process includes steps 1 to 6 as described below:

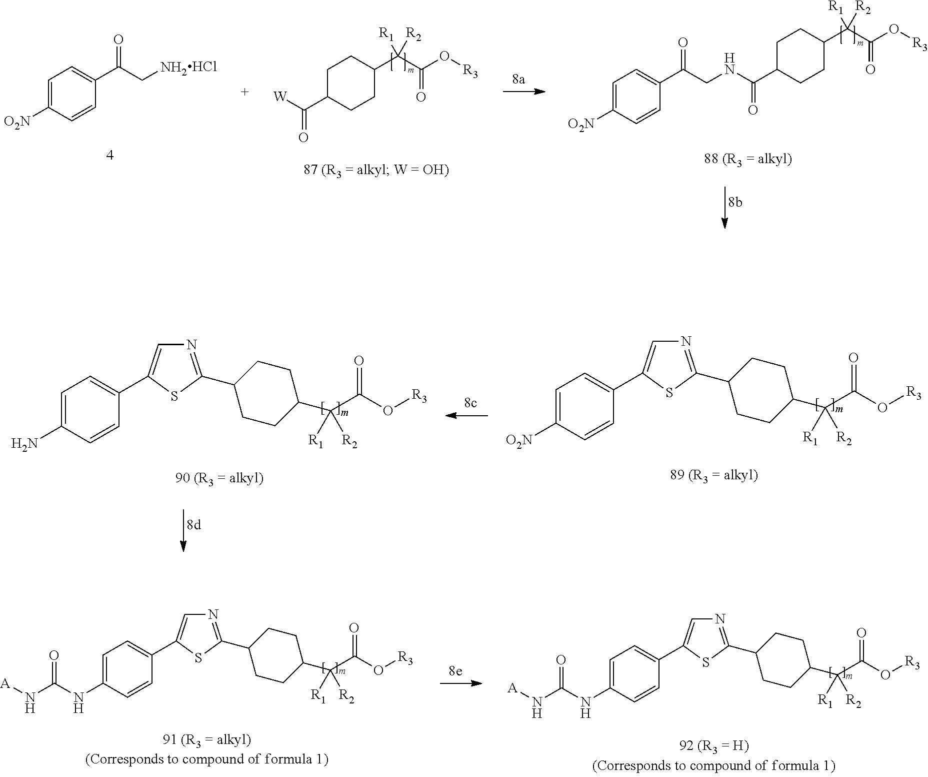

### Step 1

### Preparation of Compound of Formula 88:

The compound of formula 4 is reacted with the compound of formula 87 in presence of a coupling agent such as BOP (benzotriazol-1-yloxy)tris(dimethylamino)phosphonium hexafluorophosphate) and a suitable base such as triethylamine in a suitable solvent such as DMF or THF at a temperature range of 50° C. to 60° C., to yield the compound of formula 88 (Reaction 8a).

The compound of formula 87 is commercially available or is synthetically prepared. For example, the compound of formula 87 wherein R₃ is t-butyl and m=1 is prepared using the following scheme:

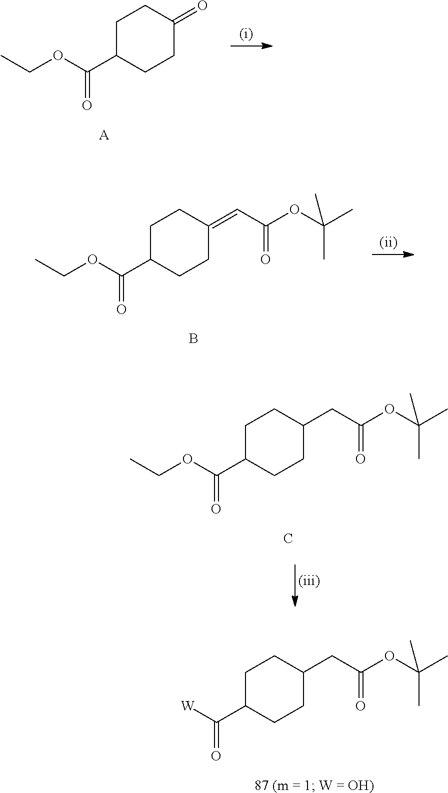

### Reaction (i):

Commercially available compound of formula A is reacted with tert-butyl-2-(diethoxy phosphoryl)acetate in presence of a suitable base such as sodium hydride in a suitable solvent such as THF at 0° C. for about 1 h, followed by at room temperature for about 16 h, to yield the compound of formula B.

### Reaction (ii):

The compound of formula B is hydrogenated in presence of suitable catalyst such as Pd/C in a suitable solvent such as ethyl acetate, ethanol or methanol at room temperature, to yield the compound of formula C.

Reaction (iii):

The compound of formula C is hydrolysed partially in presence of a suitable base such as KOH in a suitable solvent mixture such as methanol and water at room temperature for about 2 h to yield the compound of formula 87 (m=1).

Alternately, the compound of formula 88 is prepared by reaction of the compound of formula 4 with the compound of formula 87 in presence of a coupling agent such as HATU and a base such as DIPEA in suitable solvent such as DMF for 30 min to 2 h at room temperature.

### Step 2

### Preparation of Compound of Formula 89:

The compound of formula 88 is refluxed with Lawesson's reagent in a suitable solvent such as 1,4-dioxane or THF, at a temperature range of 80° C. to 110° C., to yield the compound of formula 89 (Reaction 8b).

### Step 3

### Preparation of Compound of Formula 90:

The compound of formula 89 is reduced with a suitable reducing agent such as Fe and NH₄Cl in a suitable solvent mixture of EtOH, THF and water at a temperature range of 70° C. to 80° C. for 2-6 h, to yield the compound of formula 90 (Reaction 8c).

### Step 4

### Preparation of Compound of Formula 91:

The compound of formula 90 is reacted with a compound of formula 8 (i) in a suitable solvent such as THF or dichloromethane at room temperature for 2-16 h, to yield the compound of formula 91 (Reaction 8d).

Alternately, the compound of formula 90 is reacted with the compound of formula 8 (ii) in presence of a coupling agent such as carbonyl diimidazole in a suitable solvent such as THF at room temperature for about 24 h to yield the compound of formula 91.

### Step 5

### Preparation of Compound of Formula 92:

The compound of formula 91 is hydrolysed using suitable reagent such as aqueous LiOH in a suitable solvent such as THF or methanol or a mixture thereof, at room temperature for 2-16 h at room temperature, to yield the compound of formula 92 (Reaction 8e).

### Step 6

The carboxylic acid (compound of formula 92) is optionally converted to its corresponding ester prodrugs by any suitable method well known in the art.

### Scheme 8B:

Scheme 8B depicts a process for the preparation of the compounds of formula 1 (referred in Scheme 8B as compound 93 (R₃=(C₁-C₁₂)-alkyl) and compound 94 (R₃═H), wherein Z is

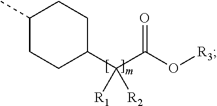

### B is

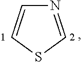

wherein 1 and 2 are the points of attachment of B to phenyl and to Z respectively; L=*NHC(S)NH, wherein * indicates the point of attachment of L to A; A, n, R₁ and R₂ are as defined in formula 1). Said process includes steps 1 to 3 as described below:

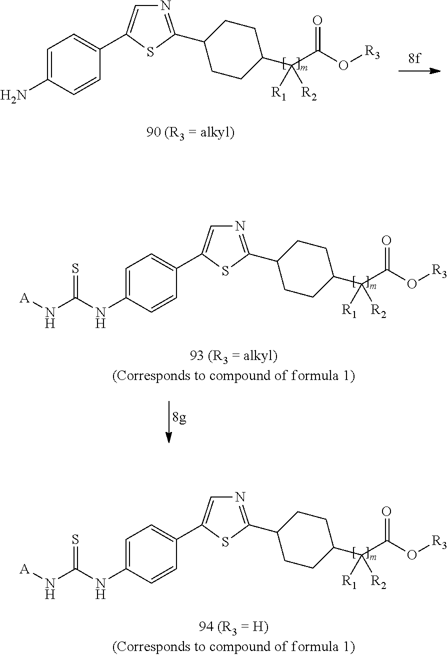

### Step 1

### Preparation of Compound of Formula 93:

The compound of formula 90 is reacted with a compound of formula 8 (iii) in a suitable solvent such as THF or dichloromethane at room temperature for 2-16 h, to yield the compound of formula 93 (Reaction 8f).

### Step 2

### Preparation of Compound of Formula 94:

The compound of formula 93 is hydrolysed using suitable reagent such as aqueous LiOH in a suitable solvent such as THF or methanol or a mixture thereof, at room temperature for 2-16 h at room temperature, to yield the compound of formula 94 (Reaction 8g).

### Step 3

The carboxylic acid (compound of formula 94) is optionally converted to its corresponding ester prodrugs by any suitable method well known in the art.

### Scheme 8C:

Scheme 8C depicts a process for the preparation of the compounds of formula 1 (referred in Scheme 8C as compound 95 (R₃═(C₁-C₁₂)-alkyl) and compound 96 (R₃═H), wherein Z is

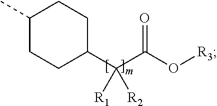

### B is

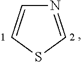

wherein 1 and 2 are the points of attachment of B to phenyl and to Z respectively; L=*CONH, wherein * indicates the point of attachment of L to A; A, n, R₁ and R₂ are as defined in formula 1). Said process includes steps 1 to 3 as described below:

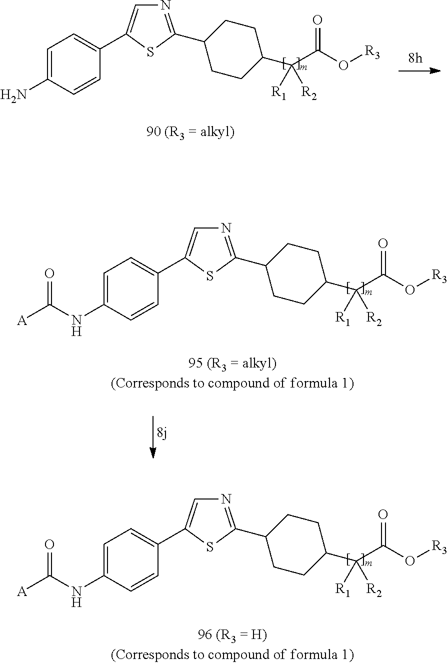

### Step 1

### Preparation of Compound of Formula 95:

The compound of formula 90 is reacted with a compound of formula 8 (iv) in a suitable solvent such as dichloromethane or chloroform in a suitable base such as pyridine at room temperature for 1-2 h, to yield the compound of formula 95 (Reaction 8h).

Alternately, the compound of formula 90 is reacted with the compound of formula 8 (v) in a suitable solvent such as toluene and a coupling agent such as trimethylaluminium to yield the compound of formula 95.

### Step 2

### Preparation of Compound of Formula 96:

The compound of formula 95 is hydrolysed using suitable reagent such as aqueous LiOH in a suitable solvent such as THF or methanol or a mixture thereof, at room temperature for 2-16 h at room temperature, to yield the compound of formula 96 (Reaction 8j).

### Step 3

The carboxylic acid (compound of formula 96) is optionally converted to its corresponding ester prodrugs by any suitable method well known in the art.

### Scheme 8D:

Scheme 8D depicts a process for the preparation of the compounds of formula 1 (referred in Scheme 8D as compound 97 (R₃═(C₁-C₁₂)-alkyl) and compound 98 (R₃═H), wherein Z is

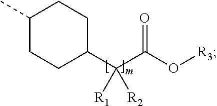

### B is

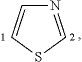

wherein 1 and 2 are the points of attachment of B to phenyl and to Z respectively; L=*SO₂NH, wherein * indicates the point of attachment of L to A; A, n, R₁, R₂ and R₃ are as defined in formula 1). Said process includes steps 1 to 3 as described below:

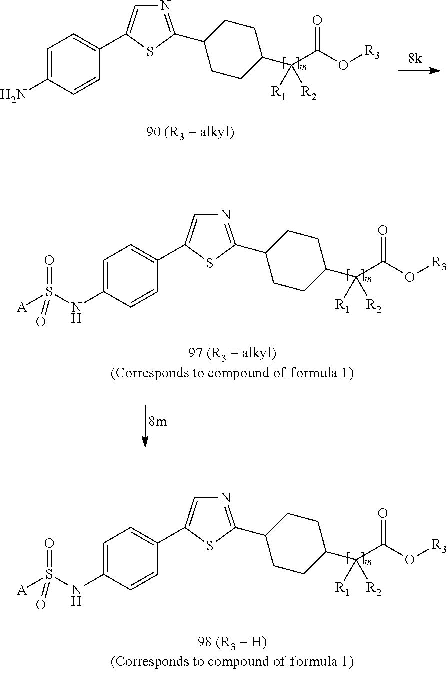

### Step 1

### Preparation of Compound of Formula 95:

The compound of formula 90 is reacted with a compound of formula 8 (vi) in a suitable solvent such as dichloromethane or chloroform in a suitable base such as pyridine at room temperature for 1-2 h, to yield the compound of formula 95 (Reaction 8k).

### Step 2

### Preparation of Compound of Formula 96:

The compound of formula 95 is hydrolysed using suitable reagent such as aqueous LiOH in a suitable solvent such as THF or methanol or a mixture thereof, at room temperature for 2-16 h at room temperature, to yield the compound of formula 96 (Reaction 8m).

### Step 3

The carboxylic acid (compound of formula 96) is optionally converted to its corresponding ester prodrugs by any suitable method well known in the art.

### Scheme 9A:

Scheme 9A depicts a process for the preparation of the compounds of formula 1 (referred in Scheme 9A as compound 101 (R₃═(C₁-C₁₂)-alkyl) and compound 102 (R₃═H), wherein Z is

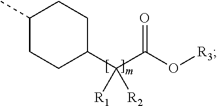

### B is

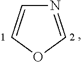

wherein 1 and 2 are the points of attachment of B to phenyl and to Z respectively; L=*NHC(O)NH, wherein * indicates the point of attachment of L to A; A, n, R₁, R₂ and R₃ are as defined in formula 1). Said process includes steps 1 to 5 as described below:

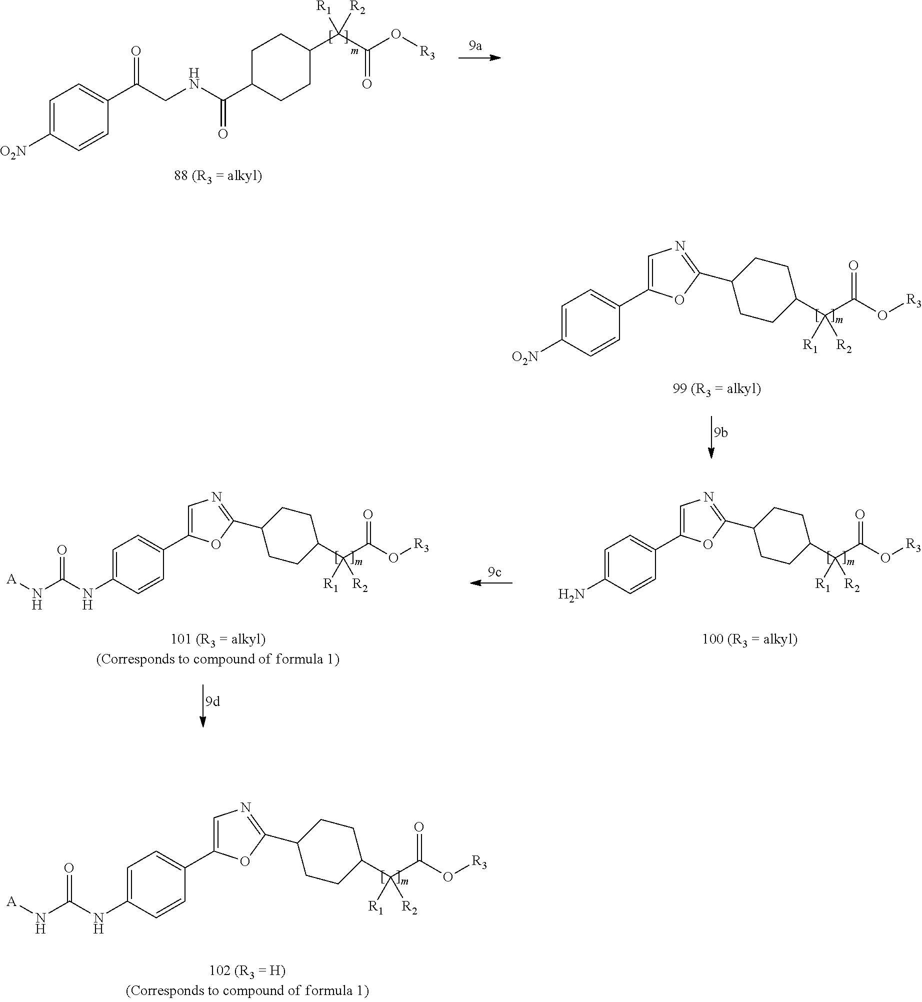

### Step 1

### Preparation of Compound of Formula 99:

The compound of formula 88 is refluxed with POCl₃, optionally in presence of solvent such as acetonitrile, at a temperature range of 80° C. to 110° C. for 2-3 h, to yield the compound of formula 99 (Reaction 9a).

### Step 2

### Preparation of Compound of Formula 100:

The compound of formula 99 is reduced with a suitable reducing agent such as Fe and NH₄Cl in a suitable solvent mixture of EtOH, THF and water at a temperature range of 70° C. to 80° C. for 2-6 h, to yield compound of formula 100 (Reaction 9b).

### Step 3

### Preparation of Compound of Formula 101:

The compound of formula 100 is reacted with a compound of formula 8 (i) in a suitable solvent such as THF or dichloromethane at room temperature for 2-16 h, to yield the compound of formula 101 (Reaction 9c).

Alternately, the compound of formula 100 is reacted with the compound of formula 8 (ii) in presence of a coupling agent such as carbonyl diimidazole in a suitable solvent such as THF at room temperature for about 24 h to yield the compound of formula 101.

### Step 4

### Preparation of Compound of Formula 102:

The compound of formula 101 is hydrolysed using suitable reagent such as aqueous LiOH in a suitable solvent such as THF or methanol or a mixture thereof, at room temperature for 2-16 h at room temperature, to yield the compound of formula 102 (Reaction 9d).

### Step 5

The carboxylic acid (compound of formula 102) is optionally converted to its corresponding ester prodrugs by any suitable method well known in the art.

### Scheme 9B:

Scheme 9B depicts a process for the preparation of the compounds of formula 1 (referred in Scheme 9B as compound 103 (R₃═(C₁-C₁₂)-alkyl) and compound 104 (R₃═H), wherein Z is

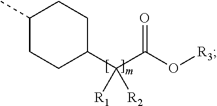

### B is

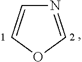

wherein 1 and 2 are the points of attachment of B to phenyl and to Z respectively; L=*NHC(S)NH, wherein * indicates the point of attachment of L to A; A, n, R₁, R₂ and R₃ are as defined in formula 1). Said process includes steps 1 to 3 as described below:

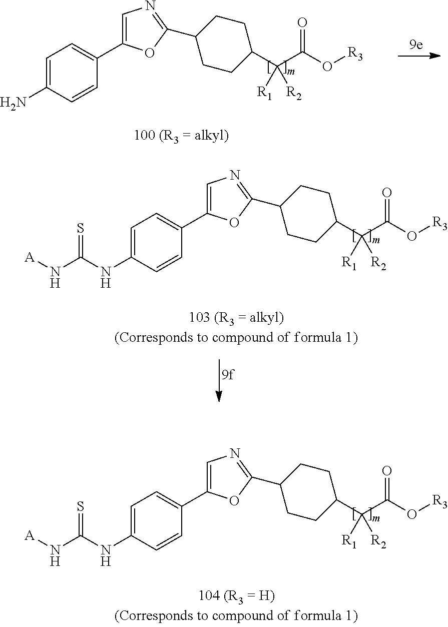

### Step 1

### Preparation of Compound of Formula 103:

The compound of formula 100 is reacted with a compound of formula 8 (iii) in a suitable solvent such as THF or dichloromethane at room temperature for 2-16 h, to yield the compound of formula 103 (Reaction 9e).

### Step 2

### Preparation of Compound of Formula 104:

The compound of formula 103 is hydrolysed using suitable reagent such as aqueous LiOH in a suitable solvent such as THF or methanol or a mixture thereof, at room temperature for 2-16 h at room temperature, to yield the compound of formula 104 (Reaction 9f).

### Step 3

The carboxylic acid (compound of formula 104) is optionally converted to its corresponding ester prodrugs by any suitable method well known in the art.

### Scheme 9C:

Scheme 9C depicts a process for the preparation of the compounds of formula 1 (referred in Scheme 9C as compound 105 (R₃═(C₁-C₁₂)-alkyl) and compound 106 (R₃═H), wherein Z is

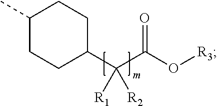

### B is

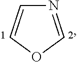

wherein 1 and 2 are the points of attachment of B to phenyl and to Z respectively; L=*NHC(O), wherein * indicates the point of attachment of L to A; A, n, R₁ and R₂ are as defined in formula 1). Said process includes steps 1 to 3 as described below:

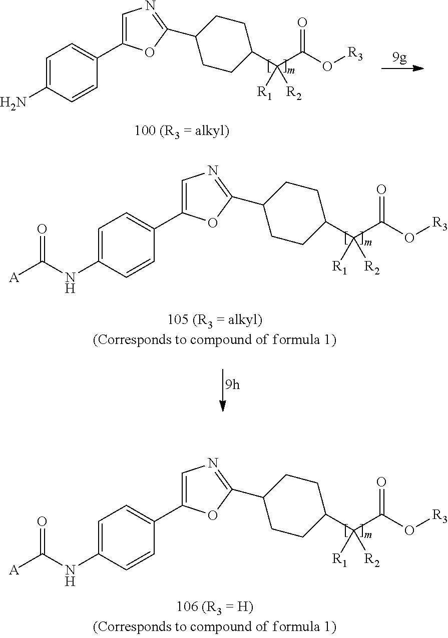

### Step 1

### Preparation of Compound of Formula 105:

The compound of formula 100 is reacted with a compound of formula 8 (iv) in a suitable solvent such as dichloromethane or chloroform in a suitable base such as pyridine at room temperature for 1-2 h, to yield the compound of formula 105 (Reaction 9g).

Alternately, the compound of formula 100 is reacted with the compound of formula 8 (v) in a suitable solvent such as toluene and a coupling agent such as trimethylaluminium to yield the compound of formula 105.

### Step 2

### Preparation of Compound of Formula 106:

The compound of formula 105 is hydrolysed using suitable reagent such as aqueous LiOH in a suitable solvent such as THF or methanol or a mixture thereof, at room temperature for 2-16 h at room temperature, to yield the compound of formula 106 (Reaction 9h).

### Step 3

The carboxylic acid (compound of formula 106) is optionally converted to its corresponding ester prodrugs by any suitable method well known in the art.

### Scheme 9D:

Scheme 9D depicts a process for the preparation of the compounds of formula 1 (referred in Scheme 9D as compound 107 (R₃═(C₁-C₁₂)-alkyl) and compound 108 (R₃═H), wherein Z is

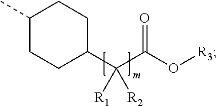

### B is

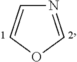

wherein 1 and 2 are the points of attachment of B to phenyl and to Z respectively; L=*SO₂NH, wherein * indicates the point of attachment of L to A; A, n, R₁, R₂ and R₃ are as defined in formula 1). Said process includes steps 1 to 3 as described below:

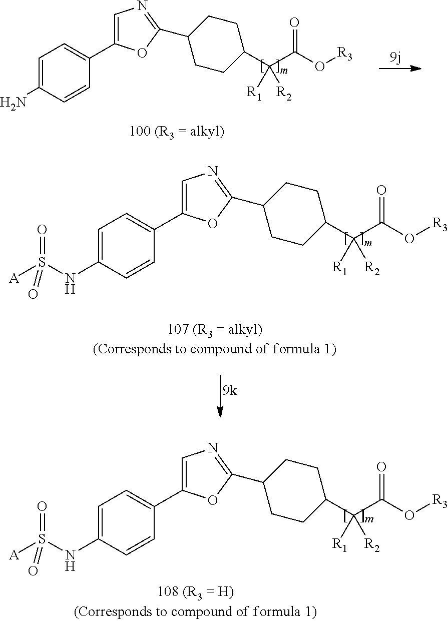

### Step 1

### Preparation of Compound of Formula 107:

The compound of formula 100 is reacted with a compound of formula 8 (vi) in a suitable solvent such as dichloromethane or chloroform in a suitable base such as pyridine at room temperature for 1-2 h, to yield the compound of formula 107 (Reaction 9j).

### Step 2

### Preparation of Compound of Formula 108:

The compound of formula 107 is hydrolysed using suitable reagent such as aqueous LiOH in a suitable solvent such as THF or methanol or a mixture thereof, at room temperature for 2-16 h at room temperature, to yield the compound of formula 108 (Reaction 9k).

### Step 3

The carboxylic acid (compound of formula 108) is optionally converted to its corresponding ester prodrugs by any suitable method well known in the art.

### Scheme 10A:

Scheme 10A depicts a process for the preparation of the compounds of formula 1 (referred in Scheme 10A as compound III (R₃═(C₁-C₁₂)-alkyl) and compound 112 (R₃═H), wherein Z is

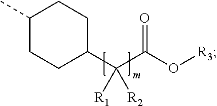

### B is

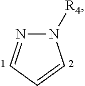

wherein 1 and 2 are the points of attachment of B to phenyl and to Z respectively; L=*NHC(O)NH, wherein * indicates the point of attachment of L to A; A, n, R₁, R₂ and R₄ are as defined in formula 1). Said process includes steps 1 to 5 as described below:

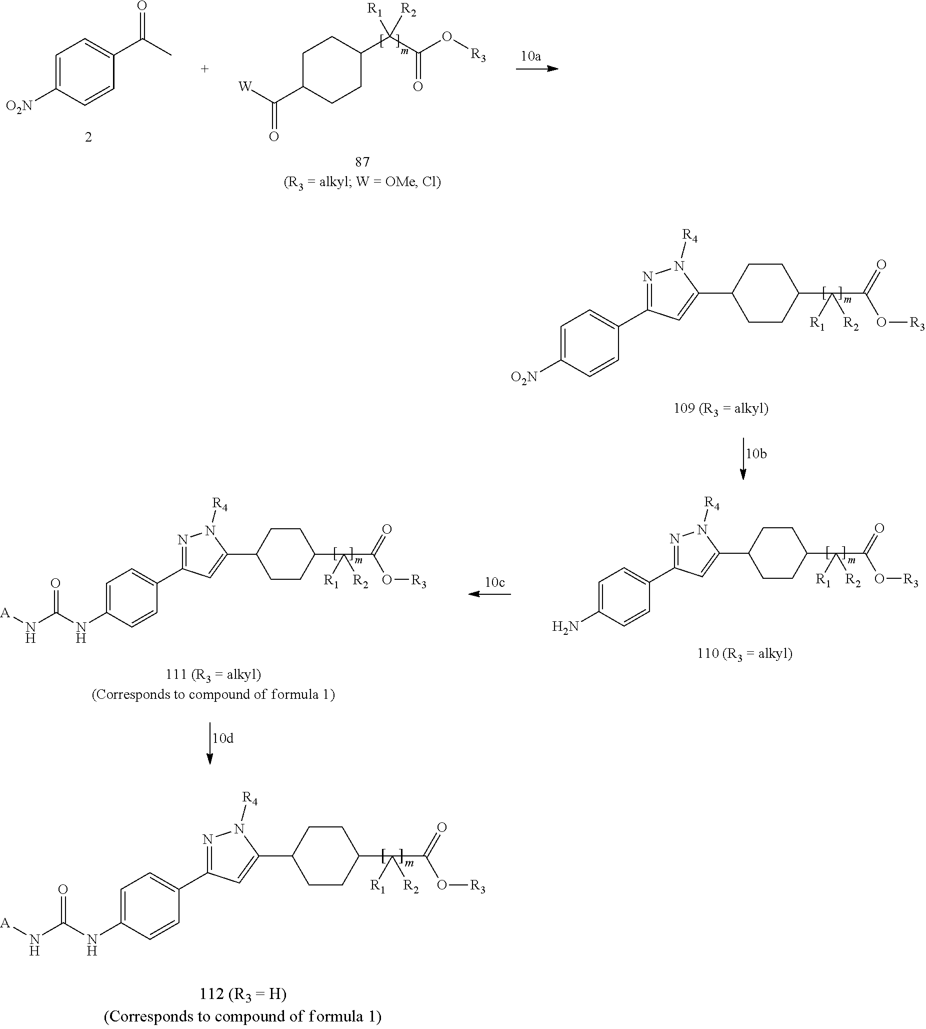

### Step 1

### Preparation of Compound of Formula 109:

Commercially available compound of formula 2 is reacted with compound of formula 87 in a suitable solvent such as toluene, ethanol or THF at a temperature range of 60° C. to 120° C., optionally in presence of a suitable base such as sodium hydride, potassium carbonate or cesium carbonate, to yield the compound of formula 87(i);

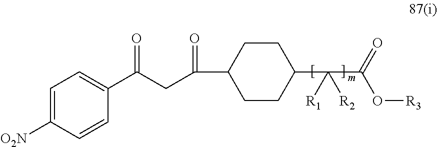

which is refluxed with compound of formula 27 (i);

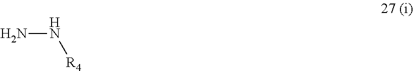

wherein R₄ is as defined in formula 1; in a suitable solvent such as ethanol or methanol at a suitable temperature of 60° C. to 85° C., to yield the compound of formula 109 (Reaction 10a).

### Step 2

### Preparation of Compound of Formula 110:

The compound of formula 109 is reduced with a suitable reducing agent such as Fe and NH₄Cl in a suitable solvent mixture of EtOH, THF and water at a temperature range of 70° C. to 80° C. for 2-6 h, to yield compound of formula 110 (Reaction 10b).

### Step 3

Preparation of compound of formula III:

The compound of formula 110 is reacted with a compound of formula 8 (i) in a suitable solvent such as THF or dichloromethane at room temperature for 2-16 h, to yield the compound of formula 111 (Reaction 10c).

Alternately, the compound of formula 110 is reacted with the compound of formula 8 (ii) in presence of a coupling agent such as carbonyl diimidazole in a suitable solvent such as THF at room temperature for 24 h, to yield the compound of formula III.

### Step 4

### Preparation of Compound of Formula 112:

The compound of formula III is hydrolysed using suitable reagent such as aqueous LiOH in a suitable solvent such as THF or methanol or a mixture thereof, at room temperature for 2-16 h at room temperature, to yield the compound of formula 112 (Reaction 10d).

### Step 5

The carboxylic acid (compound of formula 112) is optionally converted to its corresponding ester prodrugs by any suitable method well known in the art.

### Scheme 10B:

Scheme 10B depicts a process for the preparation of the compounds of formula 1 (referred in Scheme 10B as compound 113 (R₃=(C₁-C₁₂)-alkyl) and compound 114 (R₃═H), wherein Z is

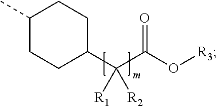

### B is

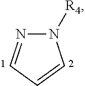

wherein 1 and 2 are the points of attachment of B to phenyl and to Z respectively; L=*NHC(S)NH, wherein * indicates the point of attachment of L to A; A, n, R₁, R₂, R₃ and R₄ are as defined in formula 1). Said process includes steps 1 to 3 as described below:

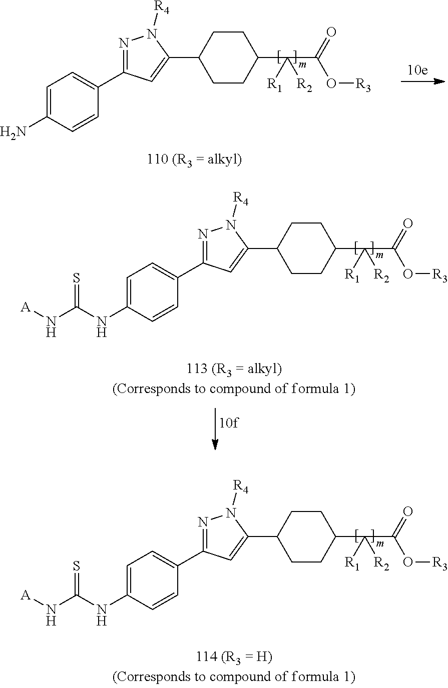

### Step 1

### Preparation of Compound of Formula 113:

The compound of formula 110 is reacted with a compound of formula 8 (iii) in a suitable solvent such as THF or dichloromethane at room temperature for 2-16 h, to yield the compound of formula 113 (Reaction 10e).

### Step 2

### Preparation of Compound of Formula 114:

The compound of formula 113 is hydrolysed using suitable reagent such as aqueous LiOH in a suitable solvent such as THF or methanol or a mixture thereof, at room temperature for 2-16 h at room temperature, to yield the compound of formula 114 (Reaction 10f).

### Step 3

The carboxylic acid (compound of formula 114) is optionally converted to its corresponding ester prodrugs by any suitable method well known in the art.

### Scheme 10C:

Scheme 10C depicts a process for the preparation of the compounds of formula 1 (referred in Scheme 10C as compound 115 (R₃═(C₁-C₁₂)-alkyl) and compound 116 (R₃═H), wherein Z is

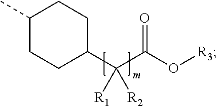

### B is

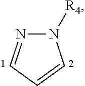

wherein 1 and 2 are the points of attachment of B to phenyl and to Z respectively; L=*CONH, wherein * indicates the point of attachment of L to A; A, n, R₁, R₂ and R₄ are as defined in formula 1). Said process includes steps 1 to 3 as described below:

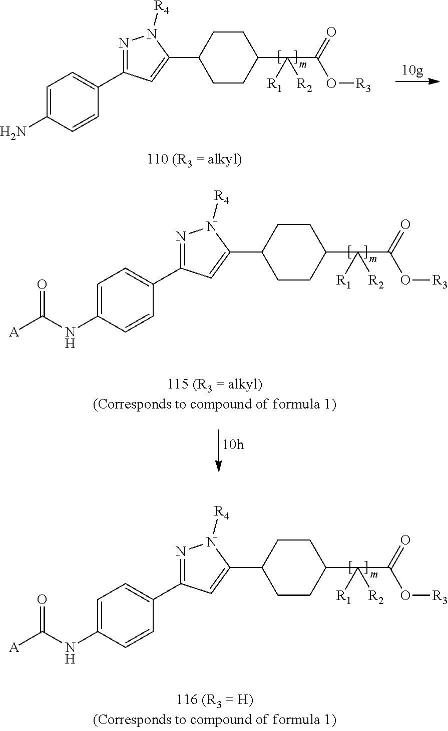

### Step 1

### Preparation of Compound of Formula 115:

The compound of formula 110 is reacted with a compound of formula 8 (iv) in a suitable solvent such as dichloromethane or chloroform in a suitable base such as pyridine at room temperature for 1-2 h, to yield the compound of formula 115 (Reaction 10g).

Alternately, the compound of formula 110 is reacted with the compound of formula 8 (v) in a suitable solvent such as toluene and a coupling agent such as trimethylaluminium to yield the compound of formula 115.

### Step 2

### Preparation of Compound of Formula 116:

The compound of formula 115 is hydrolysed using suitable reagent such as aqueous LiOH in a suitable solvent such as THF or methanol or a mixture thereof, at room temperature for 2-16 h at room temperature, to yield the compound of formula 116 (Reaction 10h).

### Step 3

The carboxylic acid (compound of formula 116) is optionally converted to its corresponding ester prodrugs by any suitable method well known in the art.

### Scheme 10D:

Scheme 10D depicts a process for the preparation of the compounds of formula 1 (referred in Scheme 10D as compound 117 (R₃═(C₁-C₁₂)-alkyl) and compound 118 (R₃═H), wherein Z is

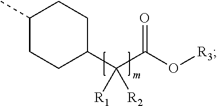

### B is

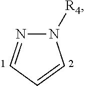

wherein 1 and 2 are the points of attachment of B to phenyl and to Z respectively; L=*SO₂NH, wherein * indicates the point of attachment of L to A; A, n, R₁, R₂ and R₄ are as defined in formula 1). Said process includes steps 1 to 3 as described below:

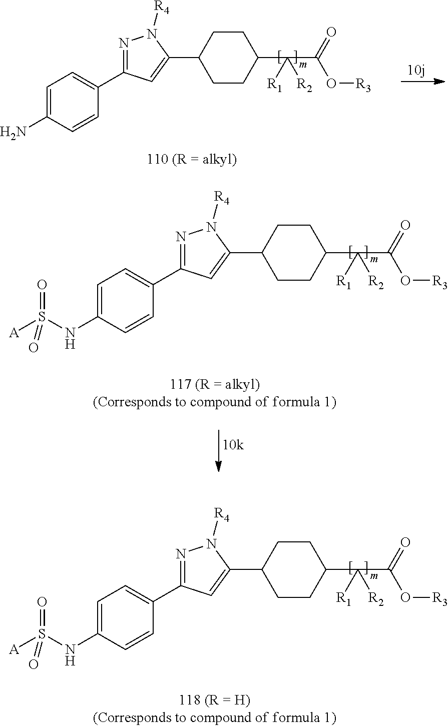

### Step 1

### Preparation of Compound of Formula 117:

The compound of formula 110 is reacted with a compound of formula 8 (vi) in a suitable solvent such as dichloromethane or chloroform in a suitable base such as pyridine at room temperature for 1-2 h, to yield the compound of formula 117 (Reaction 10j).

### Step 2

### Preparation of Compound of Formula 118:

The compound of formula 117 is hydrolysed using suitable reagent such as aqueous LiOH in a suitable solvent such as THF or methanol or a mixture thereof, at room temperature for 2-16 h at room temperature, to yield the compound of formula 118 (Reaction 10k).

### Step 3

The carboxylic acid (compound of formula 118) is optionally converted to its corresponding ester prodrugs by any suitable method well known in the art.

### Scheme 11A:

Scheme 11A depicts a process for the preparation of the compounds of formula 1 (referred in Scheme 11A as compound 124 (R₃═(C₁-C₁₂)-alkyl) and compound 125 (R₃═H), wherein Z is

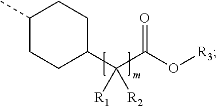

### B is

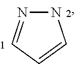

wherein 1 and 2 are the points of attachment of B to phenyl and to Z respectively; L=*NHC(O)NH, wherein * indicates the point of attachment of L to A; A, n, R₁ and R₂ are as defined in formula 1). Said process includes steps 1 to 7 as described below:

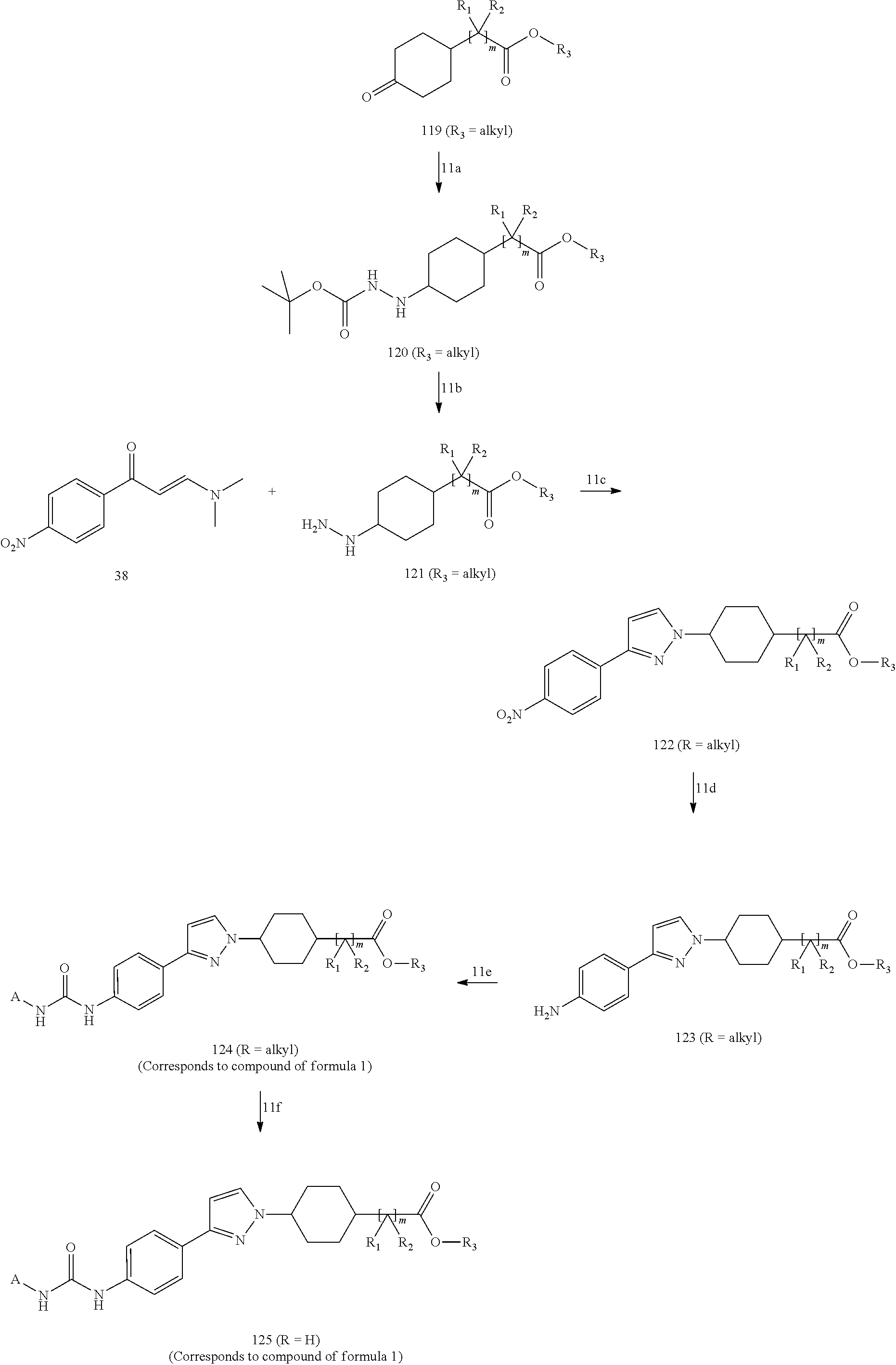

### Step 1

### Preparation of Compound of Formula 120:

Commercially available compound of formula 119 is reacted with tert-butyl carbazate followed by reaction with sodium triacetoxy borohydride or borane-THF complex at a temperature range of 0° C. to 35° C. for about 7 h, to yield the compound of formula 120 (Reaction 11a).

### Step 2

### Preparation of Compound of Formula 121:

The compound of formula 120 is treated with 4N HCl in dioxane at a temperature range of 25° C. to 50° C. for about 10 h, to yield the compound of formula 121 (Reaction 11b).

### Step 3

### Preparation of Compound of Formula 122:

The compound of formula 38 is reacted with the compound of formula 121 in a suitable solvent such as EtOH or methanol at a temperature range of 50° C. to 80° C., to yield the compound of formula 122 (Reaction 11c).

### Step 4

### Preparation of Compound of Formula 123:

The compound of formula 122 is reduced with a suitable reducing agent such as Fe and NH₄Cl in a suitable solvent mixture of EtOH, THF and water at a temperature range of 70° C. to 80° C. for 2-6 h, to yield compound of formula 123 (Reaction 11d).

### Step 5

### Preparation of Compound of Formula 124:

The compound of formula 123 is reacted with a compound of formula 8 (i) in a suitable solvent such as THF or dichloromethane at room temperature for 2-16 h, to yield the compound of formula 124 (Reaction 11e).

Alternately, the compound of formula 123 is reacted with the compound of formula 8 (ii) in presence of a coupling agent such as carbonyl diimidazole in a suitable solvent such as THF at room temperature for about 24 h to yield the compound of formula 124.

### Step 6

### Preparation of Compound of Formula 125:

The compound of formula 124 is hydrolysed using suitable reagent such as aqueous LiOH in a suitable solvent such as THF or methanol or a mixture thereof, at room temperature for 2-16 h at room temperature, to yield the compound of formula 125 (Reaction 11f).

### Step 7

The carboxylic acid (compound of formula 125) is optionally converted to its corresponding ester prodrugs by any suitable method well known in the art.

### Scheme 11B:

Scheme 11B depicts a process for the preparation of the compounds of formula 1 (referred in Scheme 11B as compound 126 (R₃═(C₁-C₁₂)-alkyl) and compound 127 (R₃═H), wherein Z is

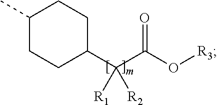

### B is

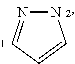

wherein 1 and 2 are the points of attachment of B to phenyl and to Z respectively; L=*NHC(S)NH, wherein * indicates the point of attachment of L to A; A, n, R₁ and R₂ are as defined in formula 1). Said process includes steps 1 to 3 as described below:

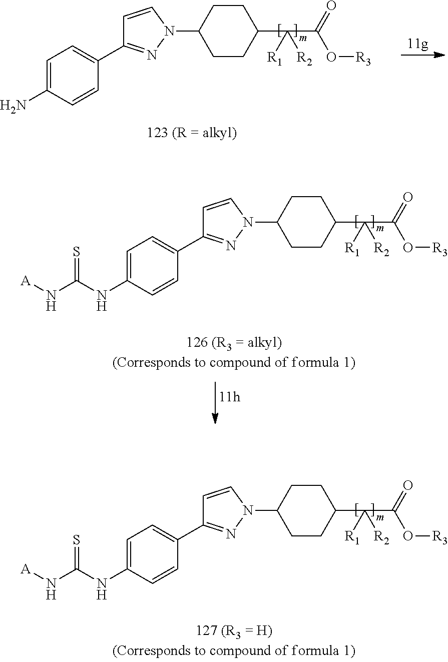

### Step 1

### Preparation of Compound of Formula 126:

The compound of formula 123 is reacted with a compound of formula 8 (iii) in a suitable solvent such as THF or dichloromethane at room temperature for 2-16 h, to yield the compound of formula 126 (Reaction 11g).

### Step 2

### Preparation of Compound of Formula 127:

The compound of formula 126 is hydrolysed using suitable reagent such as aqueous LiOH in a suitable solvent such as THF or methanol or a mixture thereof, at room temperature for 2-16 h at room temperature, to yield the compound of formula 127 (Reaction 11h).

### Step 3

The carboxylic acid (compound of formula 127) is optionally converted to its corresponding ester prodrugs by any suitable method well known in the art.

### Scheme 11C:

Scheme 11C depicts a process for the preparation of the compounds of formula 1 (referred in Scheme 11C as compound 128 (R₃=(C₁-C₁₂)-alkyl) and compound 129 (R₃═H), wherein Z is

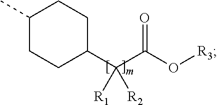

### B is

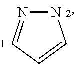

wherein 1 and 2 are the points of attachment of B to phenyl and to Z respectively; L=*CONH, wherein * indicates the point of attachment of L to A; A, n, R₁ and R₂ are as defined in formula 1). Said process includes steps 1 to 3 as described below:

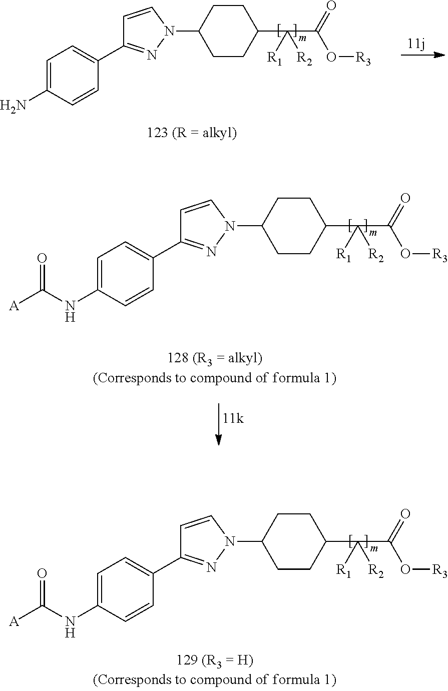

### Step 1

### Preparation of Compound of Formula 128:

The compound of formula 123 is reacted with a compound of formula 8 (iv) in a suitable solvent such as dichloromethane or chloroform in a suitable base such as pyridine at room temperature for 1-2 h, to yield the compound of formula 128 (Reaction 11j).

Alternately, the compound of formula 123 is reacted with the compound of formula 8 (v) in a suitable solvent such as toluene and a coupling agent such as trimethylaluminium to yield the compound of formula 128.

### Step 2

### Preparation of Compound of Formula 129:

The compound of formula 128 is hydrolysed using suitable reagent such as aqueous LiOH in a suitable solvent such as THF or methanol or a mixture thereof, at room temperature for 2-16 h at room temperature, to yield the compound of formula 129 (Reaction 11k).

### Step 3

The carboxylic acid (compound of formula 129) is optionally converted to its corresponding ester prodrugs by any suitable method well known in the art.

### Scheme 110:

Scheme 11 D depicts a process for the preparation of the compounds of formula 1 (referred in Scheme 11D as compound 130 (R₃═(C₁-C₁₂)-alkyl) and compound 131 (R₃═H), wherein Z is

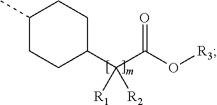

### B is

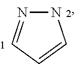

wherein 1 and 2 are the points of attachment of B to phenyl and to Z respectively; L=*SO₂NH, wherein * indicates the point of attachment of L to A; A, n, R₁ and R₂ are as defined in formula 1). Said process includes steps 1 to 3 as described below:

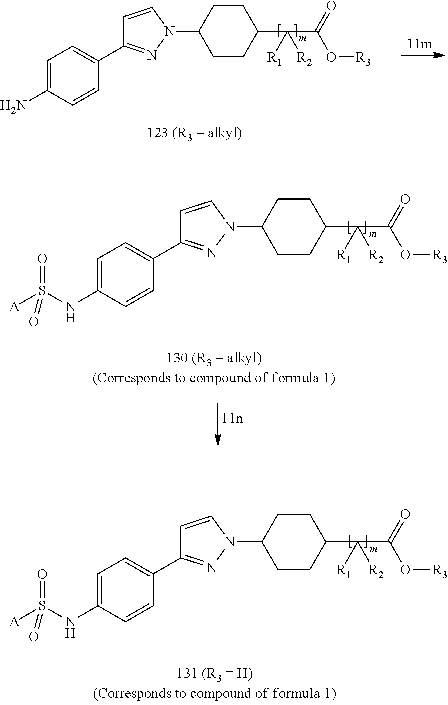

### Step 1

### Preparation of Compound of Formula 130:

The compound of formula 123 is reacted with a compound of formula 8 (vi) in a suitable solvent such as dichloromethane or chloroform in a suitable base such as pyridine at room temperature for 1-2 h, to yield the compound of formula 130 (Reaction 11m).

### Step 2

### Preparation of Compound of Formula 131:

The compound of formula 130 is hydrolysed using suitable reagent such as aqueous LiOH in a suitable solvent such as THF or methanol or a mixture thereof, at room temperature for 2-16 h at room temperature, to yield the compound of formula 131 (Reaction 11n).

### Step 3

The carboxylic acid (compound of formula 131) is optionally converted to its corresponding ester prodrugs by any suitable method well known in the art.

### Scheme 12A:

Scheme 12A depicts a process for the preparation of the compounds of formula 1 (referred in Scheme 12A as compound 135 (R₃═(C₁-C₁₂)-alkyl) and compound 136 (R₃═H), wherein Z is

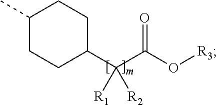

### B is

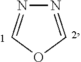

wherein 1 and 2 are the points of attachment of B to phenyl and to Z respectively; L=*NHC(O)NH, wherein * indicates the point of attachment of L to A; A, n, R₁ and R₂ are as defined in formula 1). Said process includes steps 1 to 6 as described below:

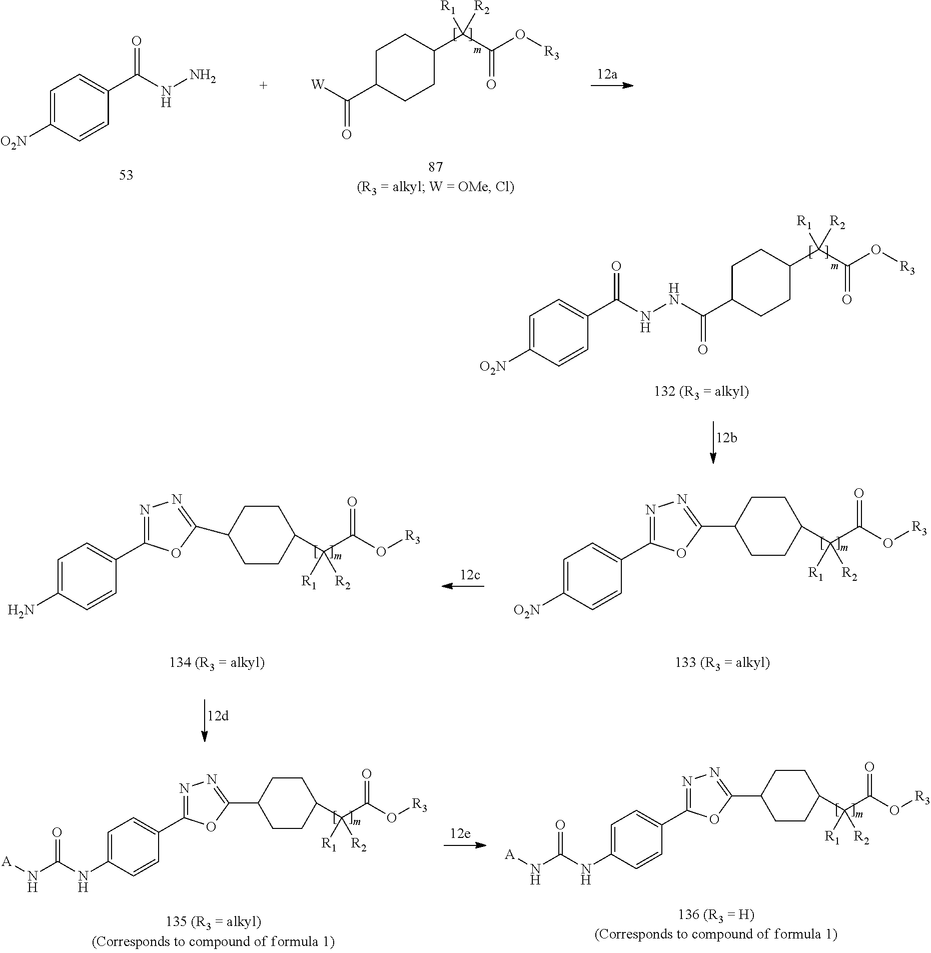

### Step 1

### Preparation of Compound of Formula 132:

The compound of formula 53 is treated with the compound of formula 87 in a suitable solvent such as dichloromethane in presence of a suitable base such as triethylamine at room temperature for 10-18 h, to yield the compound of formula 132 (Reaction 12a).

### Step 2

### Preparation of Compound of Formula 133:

The compound of formula 132 is refluxed with POCl₃, optionally in presence of solvent such as acetonitrile, at a temperature range of 80° C. to 110° C. for 2-3 h, to obtain the compound of formula 133 (Reaction 12b).

### Step 3

### Preparation of Compound of Formula 134:

The compound of formula 133 is reduced with a suitable reducing agent such as Fe and NH₄Cl in a suitable solvent mixture of EtOH, THF and water at a temperature range of 70° C. to 80° C. for 2-6 h, to yield compound of formula 134 (Reaction 12c).

### Step 4

### Preparation of Compound of Formula 135:

The compound of formula 134 is reacted with a compound of formula 8 (i) in a suitable solvent such as THF or dichloromethane at room temperature for 2-16 h, to yield the compound of formula 135 (Reaction 12d).

Alternately, the compound of formula 134 is reacted with the compound of formula 8 (ii) in presence of a coupling agent such as carbonyl diimidazole in a suitable solvent such as THF at room temperature for about 24 h to yield the compound of formula 135.

### Step 5

### Preparation of Compound of Formula 136:

The compound of formula 135 is hydrolysed using suitable reagent such as aqueous LiOH in a suitable solvent such as THF or methanol or a mixture thereof, at room temperature for 2-16 h at room temperature, to yield the compound of formula 136 (Reaction 12e).

### Step 6

The carboxylic acid (compound of formula 136) is optionally converted to its corresponding ester prodrugs by any suitable method well known in the art.

### Scheme 12B:

Scheme 12B depicts a process for the preparation of the compounds of formula 1 (referred in Scheme 12B as compound 137 (R₃=(C₁-C₁₂)-alkyl) and compound 138 (R₃═H), wherein Z is

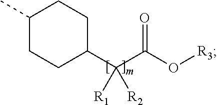

### B is

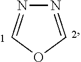

wherein 1 and 2 are the points of attachment of B to phenyl and to Z respectively; L=*NHC(S)NH, wherein * indicates the point of attachment of L to A; A, n, R₁ and R₂ are as defined in formula 1). Said process includes steps 1 to 3 as described below:

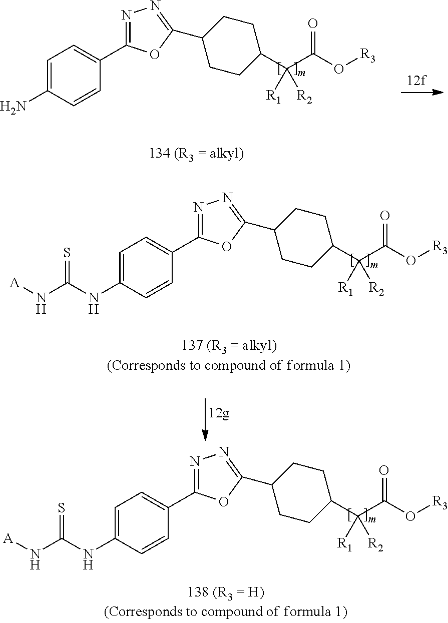

### Step 1

### Preparation of Compound of Formula 137:

The compound of formula 134 is reacted with a compound of formula 8 (iii) in a suitable solvent such as THF or dichloromethane at room temperature for 2-16 h, to yield the compound of formula 137 (Reaction 12f).

### Step 2

### Preparation of Compound of Formula 138:

The compound of formula 137 is hydrolysed using suitable reagent such as aqueous LiOH in a suitable solvent such as THF or methanol or a mixture thereof, at room temperature for 2-16 h at room temperature, to yield the compound of formula 138 (Reaction 12g).

### Step 3

The carboxylic acid (compound of formula 138) is optionally converted to its corresponding ester prodrugs by any suitable method well known in the art.

### Scheme 12C:

Scheme 12C depicts a process for the preparation of the compounds of formula 1 (referred in Scheme 12C as compound 139 (R₃═(C₁-C₁₂)-alkyl) and compound 140 (R₃═H), wherein Z is

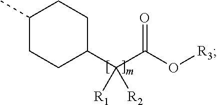

### B is

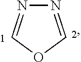

wherein 1 and 2 are the points of attachment of B to phenyl and to Z respectively; L=*CONH, wherein * indicates the point of attachment of L to A; A, n, R₁, R₂ and R₃ are as defined in formula 1). Said process includes steps 1 to 3 as described below:

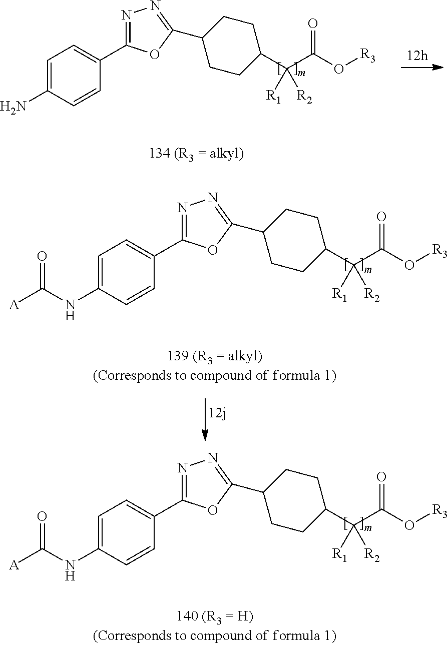

### Step 1

### Preparation of Compound of Formula 139:

The compound of formula 134 is reacted with a compound of formula 8 (iv) in a suitable solvent such as dichloromethane or chloroform in a suitable base such as pyridine at room temperature for 1-2 h, to yield the compound of formula 139 (Reaction 12h).

Alternately, the compound of formula 134 is reacted with the compound of formula 8 (v) in a suitable solvent such as toluene and a coupling agent such as trimethylaluminium to yield the compound of formula 139.

### Step 2

### Preparation of Compound of Formula 140:

The compound of formula 139 is hydrolysed using suitable reagent such as aqueous LiOH in a suitable solvent such as THF or methanol or a mixture thereof, at room temperature for 2-16 h at room temperature, to yield the compound of formula 140 (Reaction 12j).

### Step 3

The carboxylic acid (compound of formula 140) is optionally converted to its corresponding ester prodrugs by any suitable method well known in the art.

### Scheme 12D:

Scheme 12D depicts a process for the preparation of the compounds of formula 1 (referred in Scheme 12D as compound 141 (R₃═(C₁-C₁₂)-alkyl) and compound 142 (R₃═H), wherein Z is

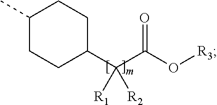

### B is

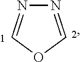

wherein 1 and 2 are the points of attachment of B to phenyl and to Z respectively; L=*SO₂NH, wherein * indicates the point of attachment of L to A; A, n, R₁ and R₃ are as defined in formula 1). Said process includes steps 1 to 3 as described below:

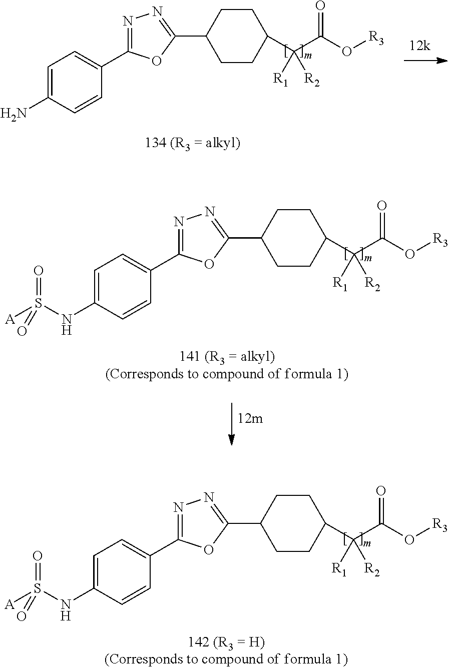

### Step 1

### Preparation of Compound of Formula 141:

The compound of formula 134 is reacted with a compound of formula 8 (vi) in a suitable solvent such as dichloromethane or chloroform in a suitable base such as pyridine at room temperature for 1-2 h, to yield the compound of formula 141 (Reaction 12k).

### Step 2

### Preparation of Compound of Formula 142:

The compound of formula 141 is hydrolysed using suitable reagent such as aqueous LiOH in a suitable solvent such as THF or methanol or a mixture thereof, at room temperature for 2-16 h at room temperature, to yield the compound of formula 142 (Reaction 12m).

### Step 3

The carboxylic acid (compound of formula 142) is optionally converted to its corresponding ester prodrugs by any suitable method well known in the art.

### Scheme 13A:

Scheme 13A depicts a process for the preparation of the compounds of formula 1 (referred in Scheme 13A as compound 146 (R₃═(C₁-C₁₂)-alkyl) and compound 147 (R₃═H), wherein Z is

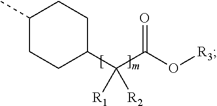

### B is

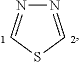

wherein 1 and 2 are the points of attachment of B to phenyl and to Z respectively; L=*NHC(O)NH, wherein * indicates the point of attachment of L to A; A, n, R₁ and R₂ are as defined in formula 1). Said process includes steps 1 to 6 as described below:

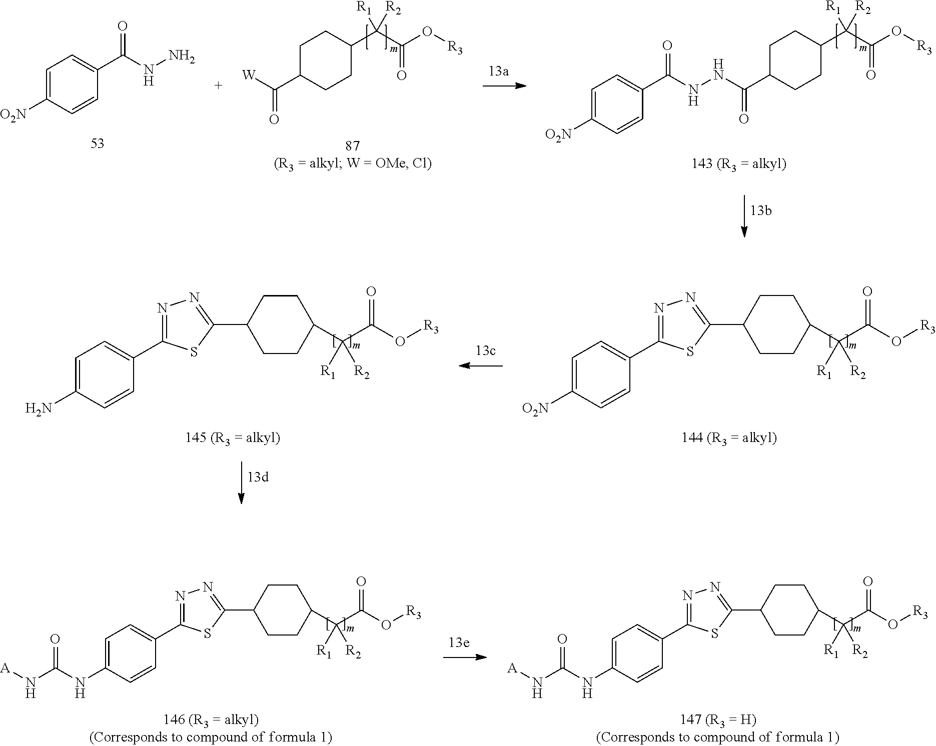

### Step 1

### Preparation of Compound of Formula 143:

The compound of formula 53 is treated with the compound of formula 87 in a suitable solvent such as dichloromethane in presence of a suitable base such as triethylamine at room temperature for 10-18 h, to yield the compound of formula 143 (Reaction 13a).

### Step 2

### Preparation of Compound of Formula 144:

The compound of formula 143 is refluxed with Lawesson's reagent in a suitable solvent such as 1,4-dioxane or THF, at a temperature range of 80° C. to 110° C., to yield the compound of formula 144 (Reaction 13b).

### Step 3

### Preparation of Compound of Formula 145:

The compound of formula 144 is reduced with a suitable reducing agent such as Fe and NH₄Cl in a suitable solvent mixture of EtOH, THF and water at a temperature range of 70° C. to 80° C. for 2-6 h, to yield compound of formula 145 (Reaction 13c).

### Step 4

### Preparation of Compound of Formula 146:

The compound of formula 145 is reacted with a compound of formula 8 (i) in a suitable solvent such as THF or dichloromethane at room temperature for 2-16 h, to yield the compound of formula 146 (Reaction 13d).

Alternately, the compound of formula 145 is reacted with the compound of formula 8 (ii) in presence of a coupling agent such as carbonyl diimidazole in a suitable solvent such as THF at room temperature for about 24 h to yield the compound of formula 146.

### Step 5

### Preparation of Compound of Formula 147:

The compound of formula 146 is hydrolysed using suitable reagent such as aqueous LiOH in a suitable solvent such as THF or methanol or a mixture thereof, at room temperature for 2-16 h at room temperature, to yield the compound of formula 147 (Reaction 13e).

### Step 6

The carboxylic acid (compound of formula 147) is optionally converted to its corresponding ester prodrugs by any suitable method well known in the art.

### Scheme 13B:

Scheme 13B depicts a process for the preparation of the compounds of formula 1 (referred in Scheme 13B as compound 148 (R₃═(C₁-C₁₂)-alkyl) and compound 149 (R₃═H), wherein Z is

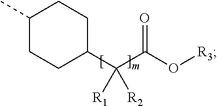

### B is

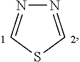

wherein 1 and 2 are the points of attachment of B to phenyl and to Z respectively; L=*NHC(S)NH, wherein * indicates the point of attachment of L to A; A, n, R₁ and R₂ are as defined in formula 1). Said process includes steps 1 to 3 as described below:

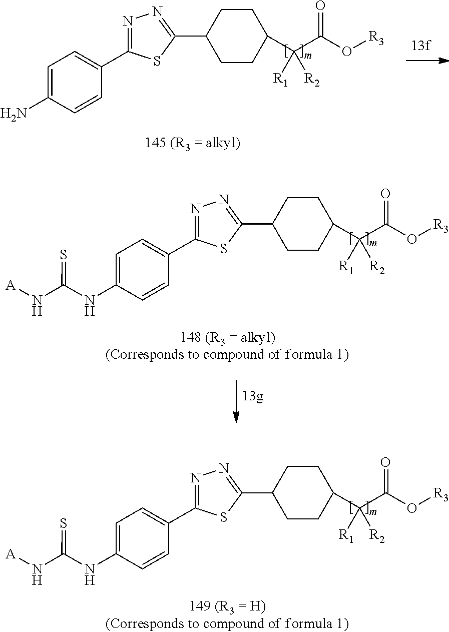

### Step 1

### Preparation of Compound of Formula 148:

The compound of formula 145 is reacted with a compound of formula 8 (iii) in a suitable solvent such as THF or dichloromethane at room temperature for 2-16 h, to yield the compound of formula 148 (Reaction 13f).

### Step 2

### Preparation of Compound of Formula 149:

The compound of formula 148 is hydrolysed using suitable reagent such as aqueous LiOH in a suitable solvent such as THF or methanol or a mixture thereof, at room temperature for 2-16 h at room temperature, to yield the compound of formula 149 (Reaction 13g).

### Step 3

The carboxylic acid (compound of formula 149) is optionally converted to its corresponding ester prodrugs by any suitable method well known in the art.

### Scheme 13C:

Scheme 13C depicts a process for the preparation of the compounds of formula 1 (referred in Scheme 13C as compound 150 (R₃═(C₁-C₁₂)-alkyl) and compound 151 (R₃═H), wherein Z is

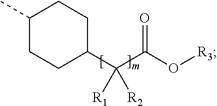

### B is

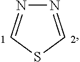

wherein 1 and 2 are the points of attachment of B to phenyl and to Z respectively; L=*NHC(O), wherein * indicates the point of attachment of L to A; A, n, R₁ and R₂ are as defined in formula 1). Said process includes steps 1 to 3 as described below:

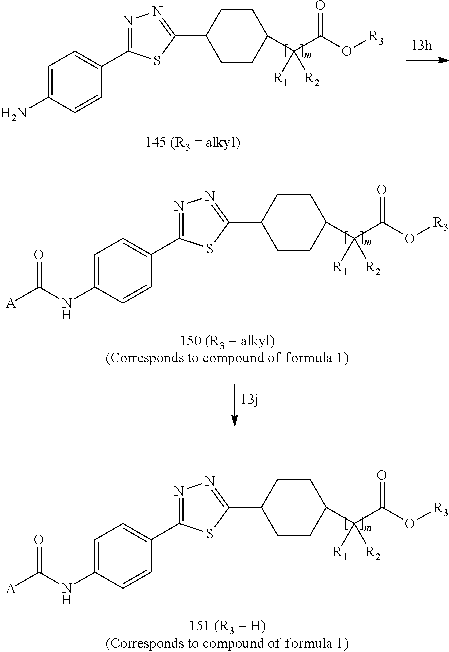

### Step 1

### Preparation of Compound of Formula 150:

The compound of formula 145 is reacted with a compound of formula 8 (iv) in a suitable solvent such as dichloromethane or chloroform in a suitable base such as pyridine at room temperature for 1-2 h, to yield the compound of formula 150 (Reaction 13h).

Alternately, the compound of formula 145 is reacted with the compound of formula 8 (v) in a suitable solvent such as toluene and a coupling agent such as trimethylaluminium to yield the compound of formula 150.

### Step 2

### Preparation of Compound of Formula 151:

The compound of formula 150 is hydrolysed using suitable reagent such as aqueous LiOH in a suitable solvent such as THF or methanol or a mixture thereof, at room temperature for 2-16 h at room temperature, to yield the compound of formula 151 (Reaction 13j).

### Step 3

The carboxylic acid (compound of formula 151) is optionally converted to its corresponding ester prodrugs by any suitable method well known in the art.

### Scheme 13D:

Scheme 13D depicts a process for the preparation of the compounds of formula 1 (referred in Scheme 13D as compound 152 (R₃═(C₁-C₁₂)-alkyl) and compound 153 (R₃═H), wherein Z is wherein Z is

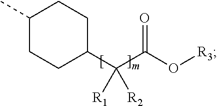

### B is

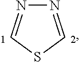

wherein 1 and 2 are the points of attachment of B to phenyl and to Z respectively; L=*NHSO₂, wherein * indicates the point of attachment of L to A; A, n, R₁ and R₂ are as defined in formula 1). Said process includes steps 1 to 3 as described below:

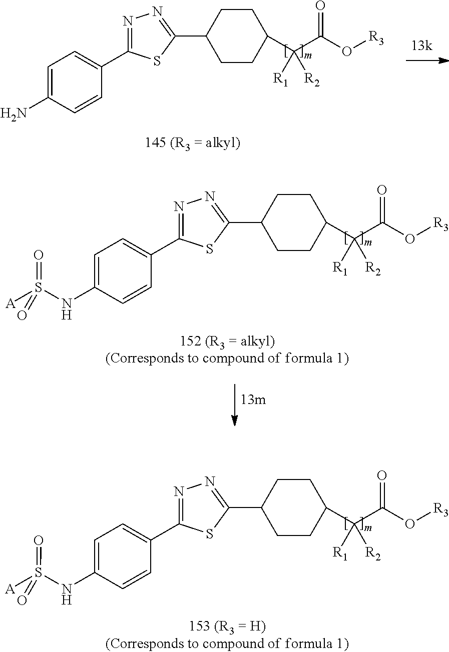

### Step 1

### Preparation of Compound of Formula 152:

The compound of formula 145 is reacted with a compound of formula 8 (vi) in a suitable solvent such as dichloromethane or chloroform in a suitable base such as pyridine at room temperature for 1-2 h, to yield the compound of formula 152 (Reaction 13k).

### Step 2

### Preparation of Compound of Formula 153:

The compound of formula 152 is hydrolysed using suitable reagent such as aqueous LiOH in a suitable solvent such as THF or methanol or a mixture thereof, at room temperature for 2-16 h at room temperature, to yield the compound of formula 153 (Reaction 13m).

### Step 3

The carboxylic acid (compound of formula 153) is optionally converted to its corresponding ester prodrugs by any suitable method well known in the art.

### Scheme 14A:

Scheme 14A depicts a process for the preparation of the compounds of formula 1 (referred in Scheme 14A as compound 156 (R₃═(C₁-C₁₂)-alkyl) and compound 157 (R₃═H), wherein Z is

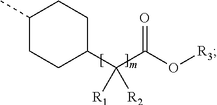

### B is

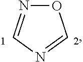

wherein 1 and 2 are the points of attachment of B to phenyl and to Z respectively; L=*NHC(O)NH, wherein * indicates the point of attachment of L to A; A, n, R₁ and R₂ are as defined in formula 1). Said process includes steps 1 to 5 as described below:

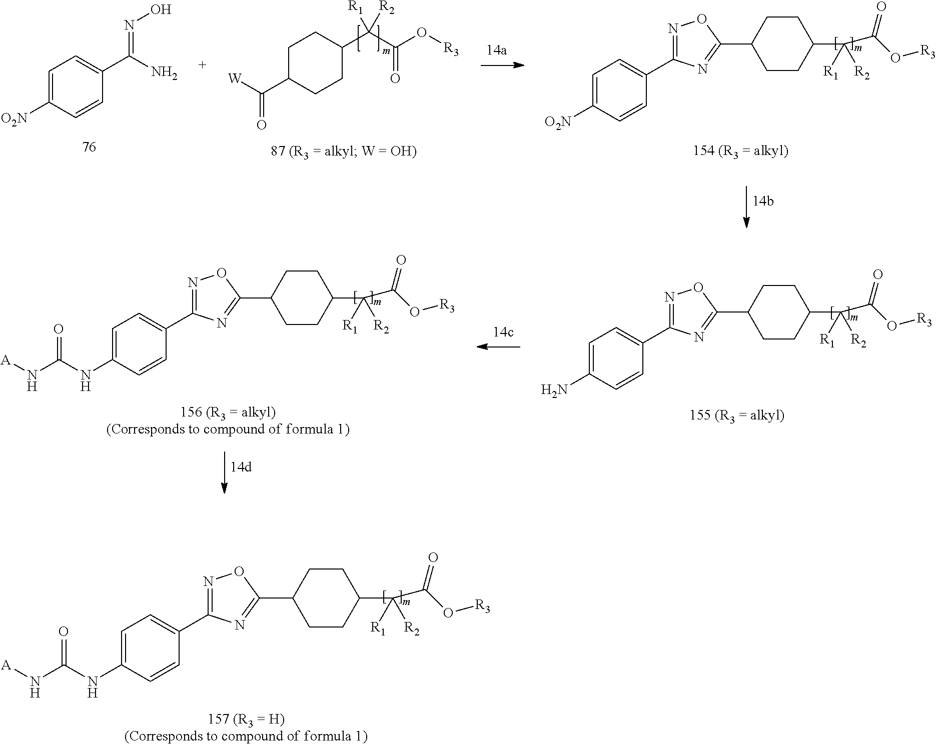

### Step 1

### Preparation of Compound of Formula 154:

The compound of formula 76 is reacted with the compound of formula 87 in a suitable solvent such as dichloromethane or chloroform in presence of a coupling reagent such as carbonylimidazole at room temperature for 8-10 h, followed by cyclisation by refluxing in a suitable solvent such as toluene at a temperature range of 100° C. to 130° C. for about 18 h, to yield the compound of formula 154 (Reaction 14a).

### Step 2

### Preparation of Compound of Formula 155:

The compound of formula 154 is reduced with a suitable reducing agent such as Fe and NH₄Cl in a suitable solvent mixture of EtOH, THF and water at a temperature range of 70° C. to 80° C. for 2-6 h, to yield compound of formula 155 (Reaction 14b).

### Step 3

### Preparation of Compound of Formula 156:

The compound of formula 155 is reacted with a compound of formula 8 (i) in a suitable solvent such as THF or dichloromethane at room temperature for 2-16 h, to yield the compound of formula 156 (Reaction 14c).

Alternately, the compound of formula 155 is reacted with the compound of formula 8 (ii) in presence of a coupling agent such as carbonyl diimidazole in a suitable solvent such as THF at room temperature for about 24 h to yield the compound of formula 156.

### Step 4

### Preparation of Compound of Formula 157:

The compound of formula 156 is hydrolysed using suitable reagent such as aqueous LiOH in a suitable solvent such as THF or methanol or a mixture thereof, at room temperature for 2-16 h at room temperature, to yield the compound of formula 157 (Reaction 14d).

### Step 5

The carboxylic acid (compound of formula 157) is optionally converted to its corresponding ester prodrugs by any suitable method well known in the art.

### Scheme 14B:

Scheme 14B depicts a process for the preparation of the compounds of formula 1 (referred in Scheme 14B as compound 158 (R₃═(C₁-C₁₂)-alkyl) and compound 159 (R₃═H), wherein Z is

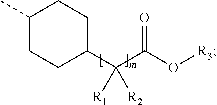

### B is

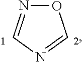

wherein 1 and 2 are the points of attachment of B to phenyl and to Z respectively; L=*NHC(S)NH, wherein * indicates the point of attachment of L to A; A, n, R₁ and R₂ are as defined in formula 1). Said process includes steps 1 to 3 as described below:

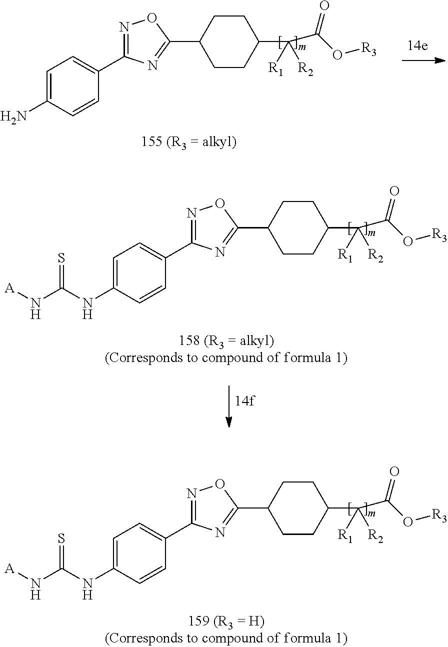

### Step 1

### Preparation of Compound of Formula 158:

The compound of formula 155 is reacted with a compound of formula 8 (iii) in a suitable solvent such as THF or dichloromethane at room temperature for 2-16 h, to yield the compound of formula 158 (Reaction 14e).

### Step 2

### Preparation of Compound of Formula 159:

The compound of formula 158 is hydrolysed using suitable reagent such as aqueous LiOH in a suitable solvent such as THF or methanol or a mixture thereof, at room temperature for 2-16 h at room temperature, to yield the compound of formula 159 (Reaction 14f).

### Step 3

The carboxylic acid (compound of formula 159) is optionally converted to its corresponding ester prodrugs by any suitable method well known in the art.

### Scheme 14C:

Scheme 14C depicts a process for the preparation of the compounds of formula 1 (referred in Scheme 14C as compound 160 (R₃═(C₁-C₁₂)-alkyl) and compound 161 (R₃═H), wherein Z is

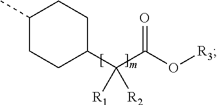

### B is

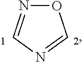

wherein 1 and 2 are the points of attachment of B to phenyl and to Z respectively; L=*CONH, wherein * indicates the point of attachment of L to A; A, n, R₁ and R₂ are as defined in formula 1). Said process includes steps 1 to 3 as described below:

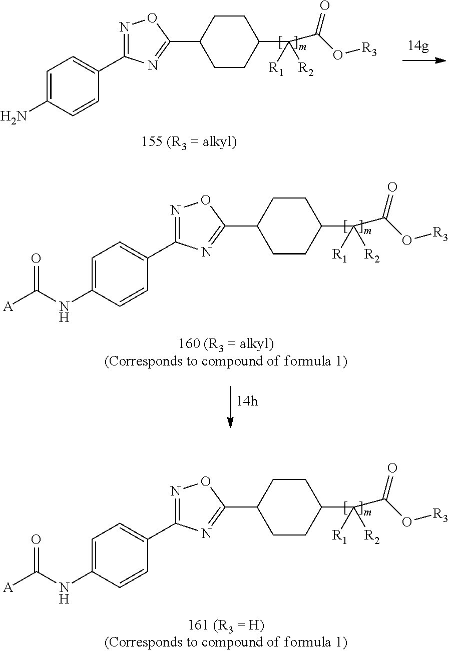

### Step 1

### Preparation of Compound of Formula 160:

The compound of formula 155 is reacted with a compound of formula 8 (iv) in a suitable solvent such as dichloromethane or chloroform in a suitable base such as pyridine at room temperature for 1-2 h, to yield the compound of formula 160 (Reaction 14g). Alternately, the compound of formula 155 is reacted with the compound of formula 8 (v) in a suitable solvent such as toluene and a coupling agent such as trimethylaluminium to yield the compound of formula 160.

### Step 2

### Preparation of Compound of Formula 161:

The compound of formula 160 is hydrolysed using suitable reagent such as aqueous LiOH in a suitable solvent such as THF or methanol or a mixture thereof, at room temperature for 2-16 h at room temperature, to yield the compound of formula 161 (Reaction 14h).

### Step 3

The carboxylic acid (compound of formula 161) is optionally converted to its corresponding ester prodrugs by any suitable method well known in the art.

### Scheme 14D:

Scheme 14D depicts a process for the preparation of the compounds of formula 1 (referred in Scheme 14D as compound 162 (R₃═(C₁-C₁₂)-alkyl) and compound 163 (R₃═H), wherein Z is

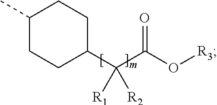

### B is

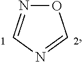

wherein 1 and 2 are the points of attachment of B to phenyl and to Z respectively; L=*SO₂NH, wherein * indicates the point of attachment of L to A; A, n, R₁ and R₂ are as defined in formula 1). Said process includes steps 1 to 3 as described below:

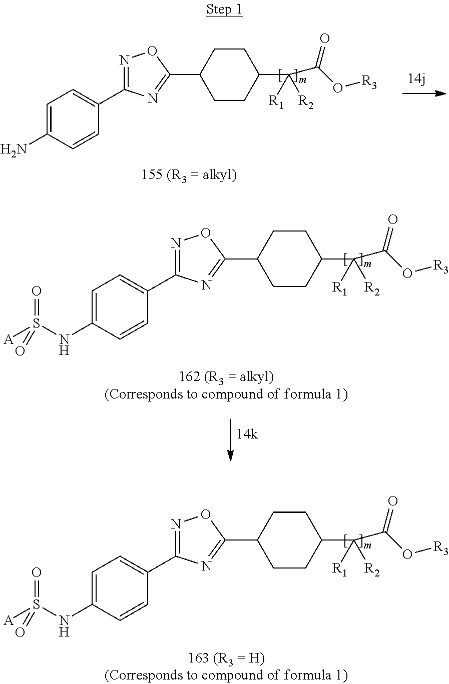

### Preparation of Compound of Formula 162:

The compound of formula 155 is reacted with a compound of formula 8 (vi) in a suitable solvent such as dichloromethane or chloroform in a suitable base such as pyridine at room temperature for 1-2 h, to yield the compound of formula 162 (Reaction 14j).

### Step 2

### Preparation of Compound of Formula 163:

The compound of formula 162 is hydrolysed using suitable reagent such as aqueous LiOH in a suitable solvent such as THF or methanol or a mixture thereof, at room temperature for 2-16 h at room temperature, to yield the compound of formula 163 (Reaction 14k).

### Step 3

The carboxylic acid (compound of formula 163) is optionally converted to its corresponding ester prodrugs by any suitable method well known in the art.

### Scheme 15:

Scheme 15 depicts a process for the preparation of the compounds of formula 1 (referred in Scheme 15 as compound 167 (L=*NHC(O)NH) and compound 168 (L=*C(O)NH), wherein Z is

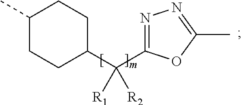

### B is

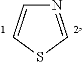

wherein 1 and 2 are the points of attachment of B to phenyl and to Z respectively. A, m, R₁ and R₂ are as defined in formula 1).Said process includes steps 1 to 5 as described below:

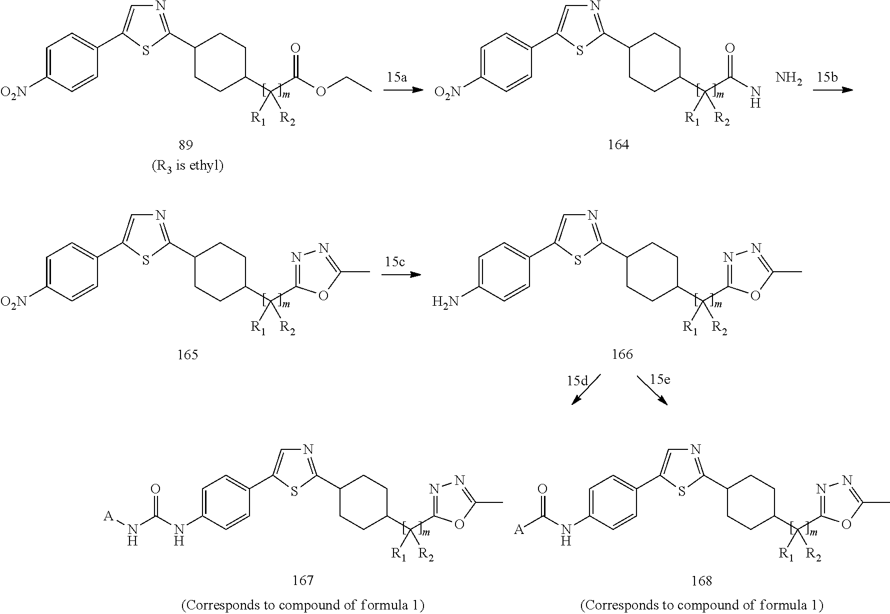

### Step 1

### Preparation of Compound of Formula 164:

The compound of formula 89 is treated with hydrazine hydrate in a suitable solvent such as ethanol at 80° C. for 4-6 h to yield the compound of formula 164 (Reaction 15a).

### Step 2

### Preparation of Compound of Formula 165:

The compound of formula 164 is heated with acetic acid and POCl₃ at 80° C. for 2-4 h to yield the compound of formula 165 (Reaction 15b).

### Step 3

### Preparation of Compound of Formula 166:

The compound of formula 165 is reduced with a suitable reducing agent such as Fe and NH₄Cl in a suitable solvent mixture of EtOH, THF and water at a temperature range of 70° C. to 80° C. for 2-6 h, to yield compound of formula 166 (Reaction 15c).

### Step 4

### Preparation of Compound of Formula 167:

The compound of formula 166 is reacted with a compound of formula 8 (i) in a suitable solvent such as THF or dichloromethane at room temperature for 2-16 h, to yield the compound of formula 167 (Reaction 15d).

Alternately, the compound of formula 166 is reacted with the compound of formula 8 (ii) in presence of a coupling agent such as carbonyl diimidazole in a suitable solvent such as THF at room temperature for 24 h to yield the compound of formula 167.

### Step 5

### Preparation of Compound of Formula 168:

The compound of formula 166 is reacted with a compound of formula 8 (iv) in a suitable solvent such as dichloromethane or chloroform in a suitable base such as pyridine at room temperature for 1-2 h, to yield the compound of formula 168 (Reaction 15e). Alternately, the compound of formula 166 is reacted with the compound of formula 8 (v) in a suitable solvent such as toluene and a coupling agent such as trimethylaluminium to yield the compound of formula 168.

### Scheme 16:

Scheme 16 depicts a process for the preparation of the compounds of formula 1 (referred in Scheme 16 as compounds 173 and 175 (L=*NHC(O)NH) and compounds 174 and 176 (L=*C(O)NH),

wherein Z is:

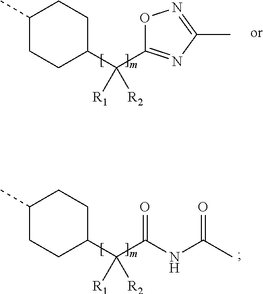

### B is

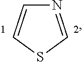

wherein 1 and 2 are the points of attachment of B to phenyl and to Z respectively. A, m, R₁ and R₂ are as defined in formula 1). Said process includes steps 1 to 9 as described below:

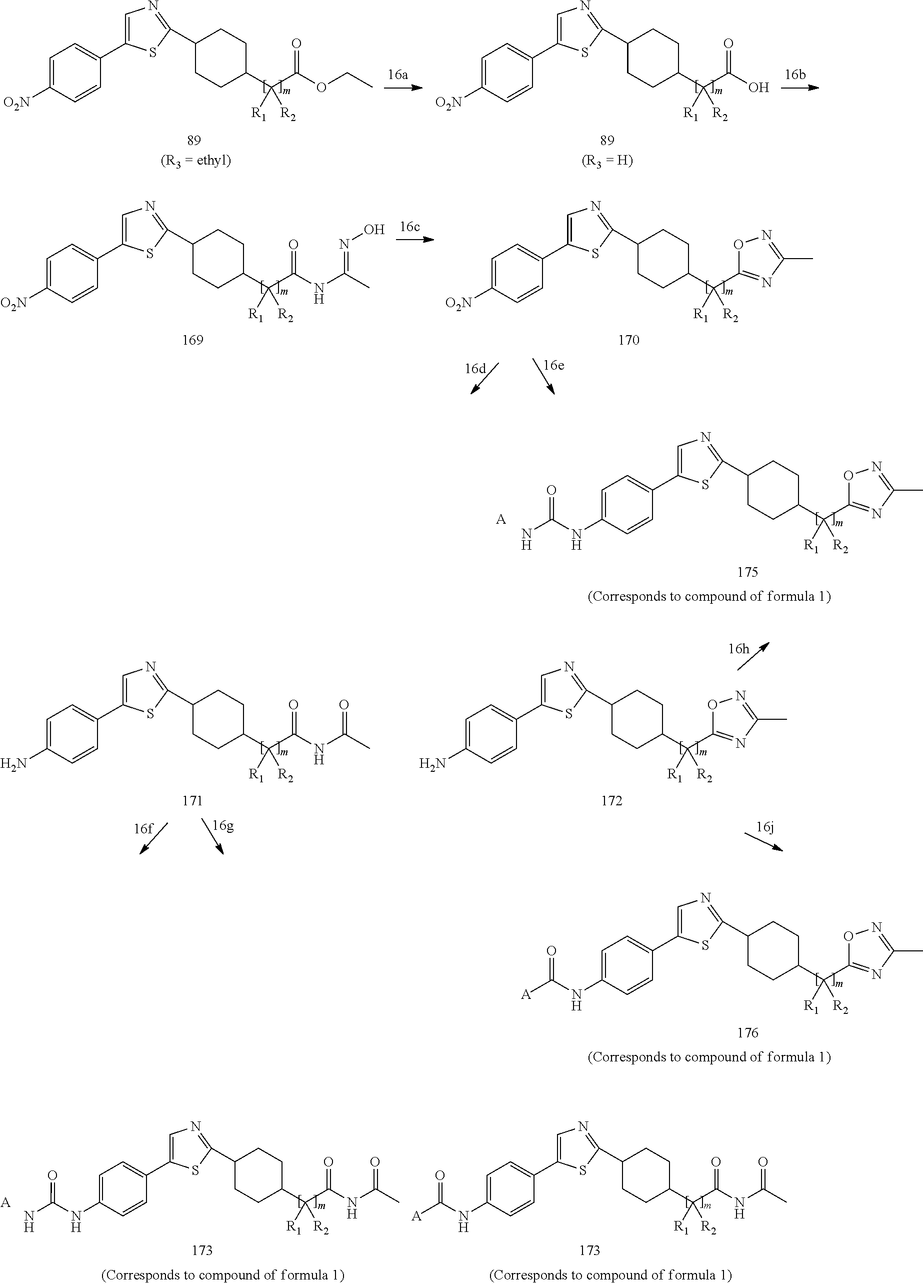

### Step 1

### Preparation of Compound of Formula 89 (R₃═H):

The compound of formula 89 (R₃=ethyl) is hydrolysed by reacting with NaOH in a suitable solvent such as a mixture of THF and methanol at room temperature for 16 h to yield compound of formula 89 (R₃═H) (Reaction 16a).

### Step 2

### Preparation of Compound of Formula 169:

The compound of formula 89 (R₃=ethyl) is reacted with oxalyl chloride and N-hydroxyacetamidine in a suitable solvent such as DCE and dioxane at room temperature for 32 h to yield compound of formula 169 (Reaction 16b).

### Step 3

### Preparation of Compound of Formula 170:

The compound of formula 169 in a suitable solvent such as DMF is heated in a microwave at 120° C. for 2-4 h to yield compound of formula 170 (Reaction 16c).

### Step 4

### Preparation of Compound of Formula 171:

The compound of formula 170 is reduced with a reducing agent such as Fe and NH₄Cl in a suitable solvent mixture of EtOH, THF and water at a temperature range of 70° C. to 80° C. for 2-6 h, to yield compound of formula 171 (Reaction 16d).

### Step 5

### Preparation of Compound of Formula 172:

The compound of formula 170 is reduced with a reducing agent such as sodium sulphide in a suitable solvent such as a mixture of dioxane and water at a temperature range of 70° C. to 90° C. for 1 h, to yield compound of formula 172 (Reaction 16e).

### Step 6

### Preparation of Compound of Formula 173:

The compound of formula 171 is reacted with a compound of formula 8 (i) in a suitable solvent such as THF or dichloromethane at room temperature for 2-16 h, to yield the compound of formula 173 (Reaction 16f).

Alternately, the compound of formula 171 is reacted with the compound of formula 8 (ii) in presence of a coupling agent such as carbonyl diimidazole in a suitable solvent such as THF at room temperature for 24 h to yield the compound of formula 173.

### Step 7

### Preparation of Compound of Formula 174:

The compound of formula 171 is reacted with a compound of formula 8 (iv) in a suitable solvent such as dichloromethane or chloroform in a suitable base such as pyridine at room temperature for 1-2 h, to yield the compound of formula 174 (Reaction 16g).

Alternately, the compound of formula 171 is reacted with the compound of formula 8 (v) in a suitable solvent such as toluene and a coupling agent such as trimethylaluminium to yield the compound of formula 174.

### Step 8

### Preparation of Compound of Formula 175:

The compound of formula 172 is reacted with a compound of formula 8 (i) in a suitable solvent such as THF or dichloromethane at room temperature for 2-16 h, to yield the compound of formula 175 (Reaction 16h).

Alternately, the compound of formula 172 is reacted with the compound of formula 8 (ii) in presence of a coupling agent such as carbonyl diimidazole in a suitable solvent such as THF at room temperature for 24 h to yield the compound of formula 175.

### Step 9

### Preparation of Compound of Formula 176:

The compound of formula 172 is reacted with a compound of formula 8 (iv) in a suitable solvent such as dichloromethane or chloroform in a suitable base such as pyridine at room temperature for 1-2 h, to yield the compound of formula 176 (Reaction 16j).

Alternately, the compound of formula 172 is reacted with the compound of formula 8 (v) in a suitable solvent such as toluene and a coupling agent such as trimethylaluminium to yield the compound of formula 176.

### Scheme 17:

Scheme 17 depicts a process for the preparation of the compounds of formula 1 (referred in Scheme 17 as compound 180 (L=*NHC(O)NH) and compound 181 (L=*C(O)NH),

wherein Z is:

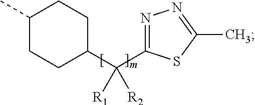

### B is

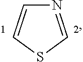

wherein 1 and 2 are the points of attachment of B to phenyl and to Z respectively. A, m, R₁ and R₂ are as defined in formula 1). Said process includes steps 1 to 5 as described below:

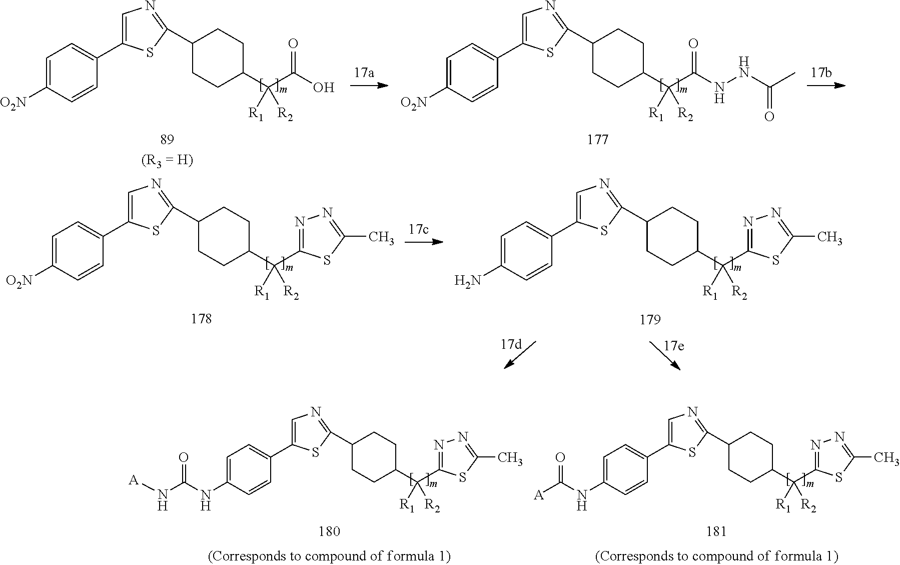

### Step 1

### Preparation of Compound of Formula 177:

The compound of formula 89 (R₃=ethyl) is reacted with oxalyl chloride and acetic hydrazide in a suitable solvent such as DCE and dioxane at room temperature for 32 h to yield compound of formula 177 (Reaction 17a).

### Step 2

### Preparation of Compound of Formula 178:

The compound of formula 177 is reacted with Lawesson's reagent in a suitable solvent such as 1,4-dioxane or xylene at a temperature range of 100° C. to 150° C., to yield compound of formula 178 (Reaction 17b).

### Step 3

### Preparation of Compound of Formula 179:

The compound of formula 178 is reduced with a reducing agent such as Fe and NH₄Cl in a suitable solvent mixture of EtOH, THF and water at a temperature range of 70° C. to 80° C. for 2-6 h, to yield compound of formula 179 (Reaction 17c).

### Step 4

### Preparation of Compound of Formula 180:

The compound of formula 179 is reacted with a compound of formula 8 (i) in a suitable solvent such as THF or dichloromethane at room temperature for 2-16 h, to yield the compound of formula 180 (Reaction 17d).

Alternately, the compound of formula 179 is reacted with the compound of formula 8 (ii) in presence of a coupling agent such as carbonyl diimidazole in a suitable solvent such as THF at room temperature for 24 h to yield the compound of formula 180.

### Step 5

### Preparation of Compound of Formula 181:

The compound of formula 179 is reacted with a compound of formula 8 (iv) in a suitable solvent such as dichloromethane or chloroform in a suitable base such as pyridine at room temperature for 1-2 h, to yield the compound of formula 181 (Reaction 17e).

Alternately, the compound of formula 179 is reacted with the compound of formula 8 (v) in a suitable solvent such as toluene and a coupling agent such as trimethylaluminium to yield the compound of formula 181.

### Scheme 18:

Scheme 18 depicts a process for the preparation of the compounds of formula 1 (referred in Scheme 18 as compound 182, 183 and 185,

wherein Z is:

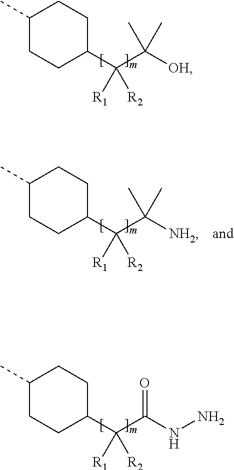

### B is

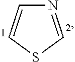

wherein 1 and 2 are the points of attachment of B to phenyl and to Z respectively; L is *NHC(O)NH; A, m, n, R₁ and R₂ are as defined in formula 1). Said process includes steps 1 to 4 as described below:

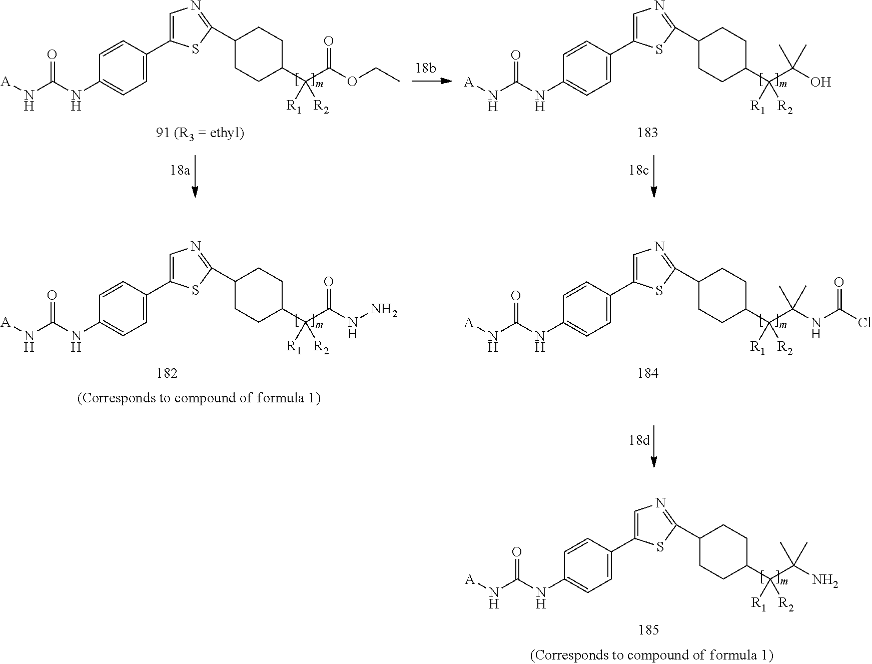

### Step 1

### Preparation of Compound of Formula 182:

The compound of formula 91 (R₃=ethyl) is reacted with hydrazine hydrate in a suitable solvent such as ethanol at a temperature of 80° C. for 5 h to yield the compound of formula 182 (Reaction 18a).

### Step 2

### Preparation of Compound of Formula 183:

The compound of formula 91 (R₃=ethyl) is treated with methyl magnesium bromide in a suitable solvent such as toluene at a temperature range from 5° C. to room temperature for 16 h to yield compound of formula 183 (Reaction 18b).

### Step 3

### Preparation of Compound of Formula 184:

The compound of formula 183 is reacted with 2-chloroacetonitrile in acetic acid as a solvent in presence of sulfuric acid at a temperature range of 10° C. to room temperature for 16 h to yield compound of formula 184 (Reaction 18c).

### Step 4

### Preparation of Compound of Formula 185:

The compound of formula 184 is reacted with thiourea in a suitable solvent such as ethanol in acetic acid at a temperature range of 70° C. to 90° C. for 2-4 h to yield compound of formula 185 (Reaction 18d).

### Scheme 19:

Scheme 19 depicts a process for the preparation of the compounds of formula 1 (referred in Scheme 19 as compound 193 (L is *NHC(O)NH), compound 194 (L is *C(O)NH), compound 195 (L is *SO₂NH), compound 196 (L is *NHC(S)NH), and compound 197 (*NHC(NR₆)NH);

wherein Z is:

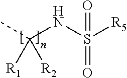

### B is

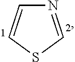

wherein 1 and 2 are the points of attachment of B to phenyl and to Z respectively; A, n, R₁, R₂, R₅ and R₆ are as defined in formula 1). Said process includes steps 1 to 11 as described below:

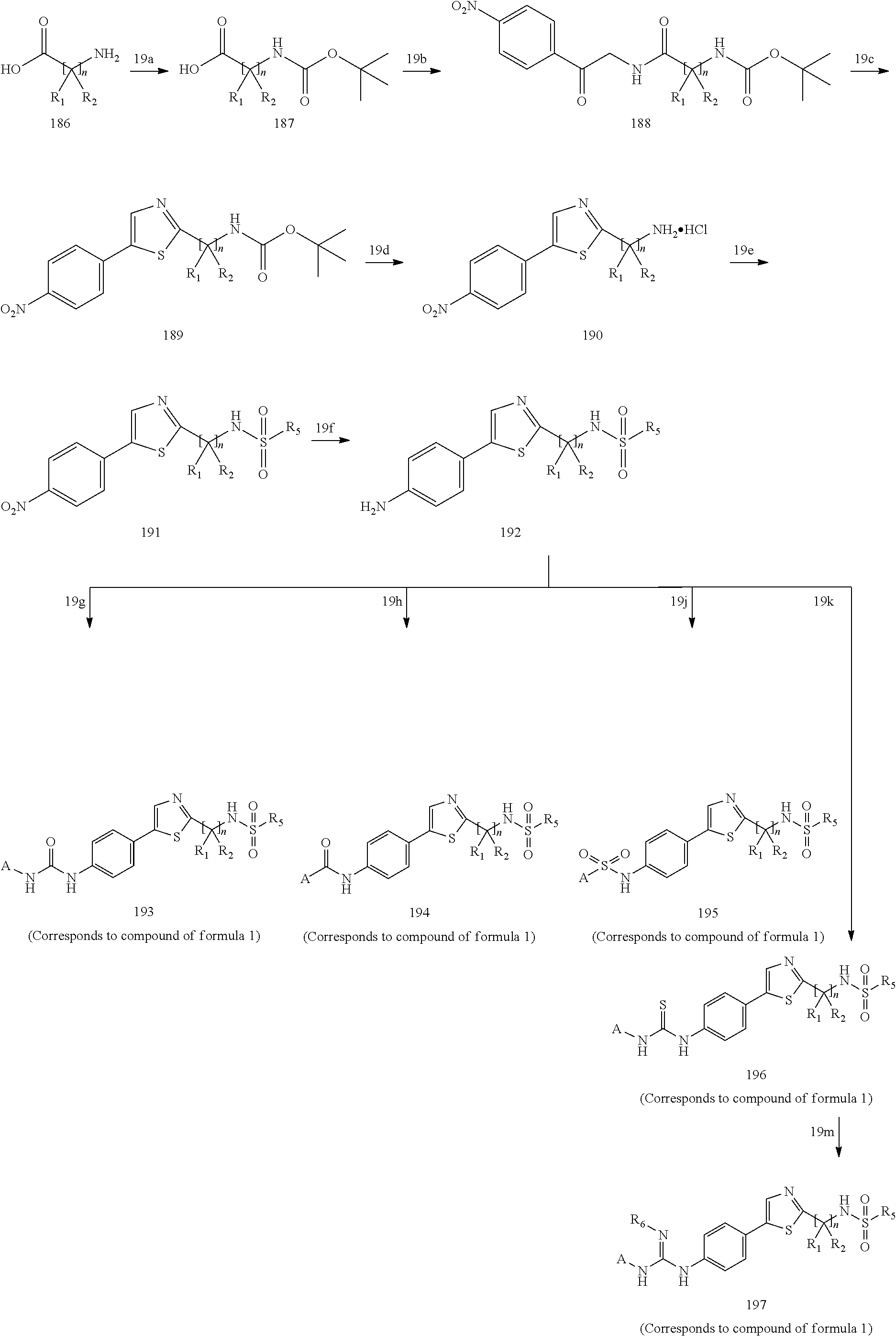

### Step 1

### Preparation of Compound of Formula 187:

Commercially available compound of formula 186 is reacted with BOC-anhydride in presence of a suitable base such as NaHCO₃ in a suitable solvent such as a mixture of acetonitrile and water at a temperature range of 0° C. to room temperature for 16 h to yield compound of formula 187 (Reaction 19a).

### Step 2

### Preparation of Compound of Formula 188:

The compound of formula 187 is reacted with 2-amino-1-(4-nitrophenyl)ethanone hydrochloride in presence of a base such as a mixture of HATU and triethylamine in a suitable solvent such as DMF at room temperature for 3-5 h to yield compound of formula 188 (Reaction 19b).

### Step 3

### Preparation of Compound of Formula 189:

The compound of formula 188 is refluxed with a reagent such as Lawesson's reagent in a suitable solvent such as 1,4-dioxane or THF, at a temperature range of 60° C. to 110° C. for 1-3 h, to yield the compound of formula 189 (Reaction 19c).

### Step 4

### Preparation of Compound of Formula 190:

The compound of formula 189 is reacted with HCl in 1,4-dioxane at room temperature for 20 h to yield compound of formula 190 (Reaction 19d).

### Step 5

### Preparation of Compound of Formula 191:

The compound of formula 190 is reacted with the reagent:

R₅SO₂Cl or (R₅SO₂)₂O,

wherein R₅ is as defined in formula 1;
in presence of a base such as triethylamine in a suitable solvent such as dichloromethane at room temperature for 1-3 h to yield compound of formula 191 (Reaction 19e).

### Step 6

### Preparation of Compound of Formula 192:

The compound of formula 191 is reduced with a reducing agent such as Fe and NH₄Cl in a suitable solvent mixture of EtOH, THF and water at a temperature range of 70° C. to 80° C. for 2-6 h, to yield compound of formula 192 (Reaction 19f).

### Step 7

### Preparation of Compound of Formula 193:

The compound of formula 192 is reacted with a compound of formula 8 (i) in a suitable solvent such as THF or dichloromethane at room temperature for 2-16 h, to yield the compound of formula 193 (Reaction 19g).

Alternately, the compound of formula 192 is reacted with the compound of formula 8 (ii) in presence of a coupling agent such as carbonyl diimidazole in a suitable solvent such as THF at room temperature for 24 h to yield the compound of formula 193.

### Step 8

### Preparation of Compound of Formula 194:

The compound of formula 192 is reacted with a compound of formula 8 (iv) in a suitable solvent such as dichloromethane or chloroform in a suitable base such as pyridine or triethylamine at room temperature for 1-2 h, to yield the compound of formula 194 (Reaction 19h).

Alternately, the compound of formula 192 is reacted with the compound of formula 8 (v) in a suitable solvent such as toluene and a coupling agent such as trimethylaluminium to yield the compound of formula 194.

### Step 9

### Preparation of Compound of Formula 195:

The compound of formula 192 is reacted with commercially available compound of formula 8 (vi) in a suitable solvent such as dichloromethane or chloroform in a suitable base such as pyridine or triethylamine at room temperature for 1-2 h, to yield the compound of formula 15 (Reaction 19j) to yield compound of formula 195 (Reaction 19j).

### Step 10

### Preparation of Compound of Formula 196:

The compound of formula 192 is reacted with commercially available compound of formula 8 (iii) in a suitable solvent such as THF or dichloromethane at room temperature for 2-16 h, to yield the compound of formula 196 (Reaction 19k).

### Step 11

### Preparation of Compound of Formula 197:

The compound of formula 196 is reacted with the reagent:

R₆—NH₂,

wherein R₆ is as defined in formula 1;
in presence of HgO in a suitable solvent such as methanol at room temperature for 1-3 h to yield compound of formula 197.

### Scheme 20:

Scheme 20 depicts a process for the preparation of the compounds of formula 1 (referred in Scheme 20 as compound 200 (L is *NHC(O)NH), compound 202 (L is *C(O)NH) and compound 204 (L is *SO₂NH);

wherein Z is:

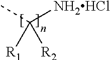

### B is

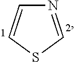

wherein 1 and 2 are the points of attachment of B to phenyl and to Z respectively; A, n, R₁, R₂ and R₅ are as defined in formula 1). Said process includes steps 1 to 7 as described below:

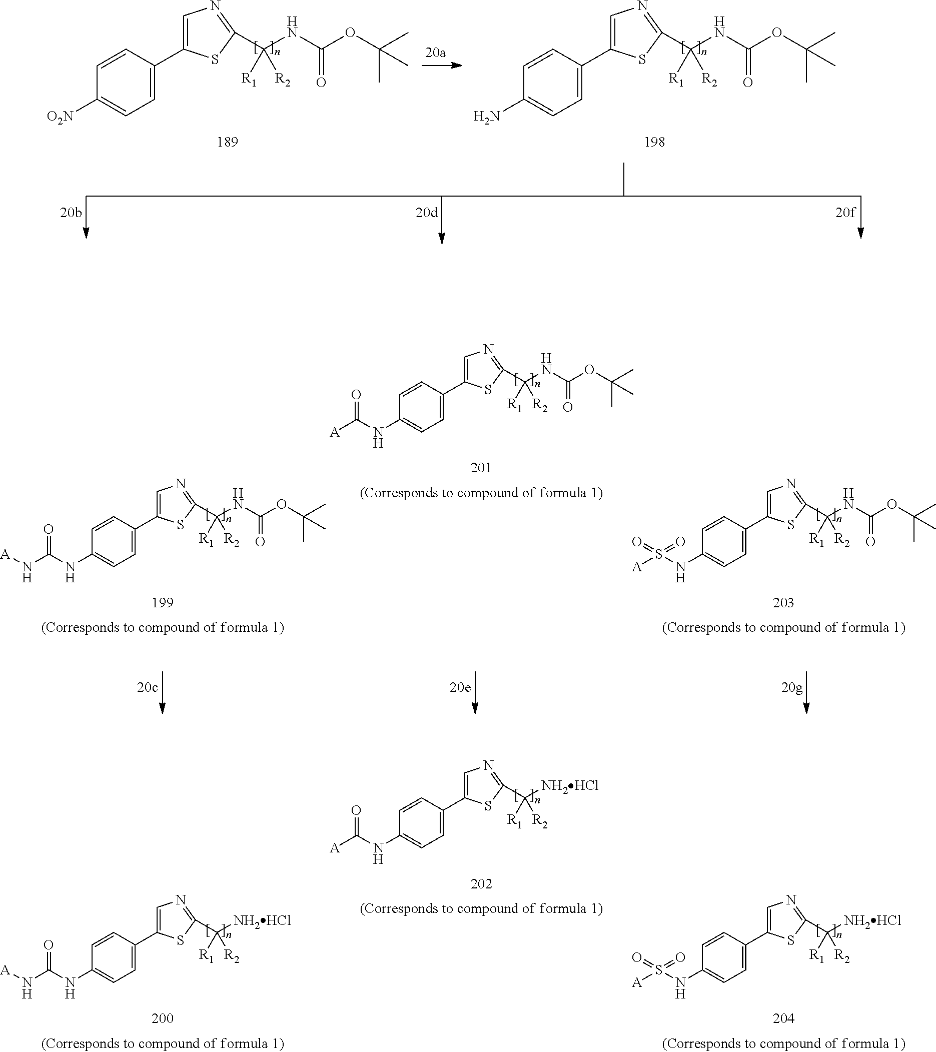

### Step 1

### Preparation of Compound of Formula 198:

The compound of formula 189 is reduced with a reducing agent such as Fe and NH₄Cl in a suitable solvent mixture of EtOH, THF and water at a temperature range of 70° C. to 80° C. for 2-6 h, to yield compound of formula 198 (Reaction 20a).

### Step 2

### Preparation of Compound of Formula 199:

The compound of formula 198 is reacted with a compound of formula 8 (i) in a suitable solvent such as THF or dichloromethane at room temperature for 2-16 h, to yield the compound of formula 199 (Reaction 20b).

Alternately, the compound of formula 198 is reacted with the compound of formula 8 (ii) in presence of a coupling agent such as carbonyl diimidazole in a suitable solvent such as THF at room temperature for 24 h to yield the compound of formula 199.

### Step 3

### Preparation of Compound of Formula 200:

The compound of formula 199 is treated with HCl in a suitable solvent such as 1,4-dioxane at room temperature for 16-24 h to yield compound of formula 200 (Reaction 20c).

### Step 4

### Preparation of Compound of Formula 201:

The compound of formula 198 is reacted with a compound of formula 8 (iv) in a suitable solvent such as dichloromethane or chloroform in a suitable base such as pyridine or triethylamine at room temperature for 1-2 h, to yield the compound of formula 201 (Reaction 20d).

Alternately, the compound of formula 198 is reacted with the compound of formula 8 (v) in a suitable solvent such as toluene and a coupling agent such as trimethylaluminium to yield the compound of formula 201.

### Step 5

### Preparation of Compound of Formula 202:

The compound of formula 201 is treated with HCl in a suitable solvent such as 1,4-dioxane at room temperature for 16-24 h to yield compound of formula 202 (Reaction 20e).

### Step 6

### Preparation of Compound of Formula 203:

The compound of formula 198 is reacted with commercially available compound of formula 8 (vi) in a suitable solvent such as dichloromethane or chloroform in a suitable base such as pyridine or triethylamine at room temperature for 1-2 h, to yield the compound of formula 203 (Reaction 20f).

### Step 7

### Preparation of Compound of Formula 204:

The compound of formula 203 is treated with HCl in a suitable solvent such as 1,4-dioxane at room temperature for 16-24 h to yield compound of formula 204 (Reaction 20g).

### Scheme 21:

Scheme 21 depicts a process for the preparation of the compounds of formula 1 (referred in Scheme 21 as compound 207 (L is *NHC(O)NH), compound 208 (L is *C(O)NH) and compound 209 (L is *SO₂NH);

wherein Z is:

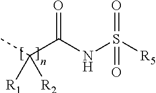

### B is

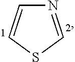

wherein 1 and 2 are the points of attachment of B to phenyl and to Z respectively; A, n, R₁, R₂ and R₅ are as defined in formula 1). Said process includes steps 1 to 6 as described below:

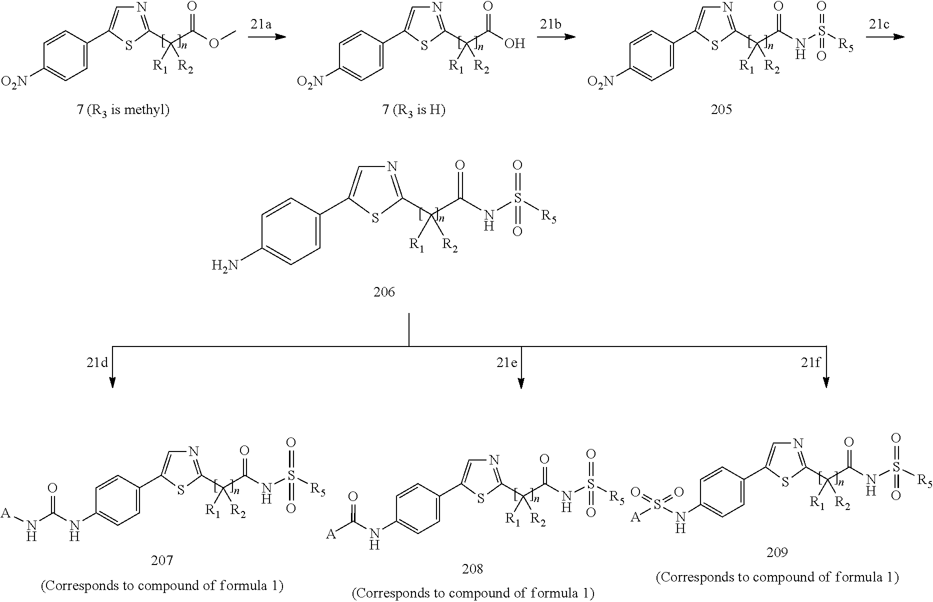

### Step 1

### Preparation of Compound of Formula 7 (R₃ is H):

The compound of formula 7 (R₃ is methyl) is hydrolysed using 1N NaOH in a suitable solvent such as a mixture of THF and methanol at room temperature for 16-24 h to yield compound of formula 7 (R₃ is H) (Reaction 21a).

### Step 2

### Preparation of Compound of Formula 205:

The compound of formula 7 (R₃ is H) is refluxed with the reagent:

R₅SO₂NH₂,

wherein R₅ is defined in formula 1;
in presence of isobutyl chloroformate in presence of bases such as N-Methyl morpholine and DBU in a suitable solvent such as THF for 16 h to yield compound of formula 205 (Reaction 21b).

### Step 3

### Preparation of Compound of Formula 206:

The compound of formula 205 is reduced with a reducing agent such as Fe and NH₄Cl in a suitable solvent mixture of EtOH, THF and water at a temperature range of 70° C. to 80° C. for 2-6 h, to yield compound of formula 206 (Reaction 21c).

### Step 4

### Preparation of Compound of Formula 207:

The compound of formula 206 is reacted with a compound of formula 8 (i) in a suitable solvent such as THF or dichloromethane at room temperature for 2-16 h, to yield the compound of formula 207 (Reaction 21d).

Alternately, the compound of formula 206 is reacted with the compound of formula 8 (ii) in presence of a coupling agent such as carbonyl diimidazole in a suitable solvent such as THF at room temperature for 24 h.

### Step 5

### Preparation of Compound of Formula 208:

The compound of formula 206 is reacted with a compound of formula 8 (iv) in a suitable solvent such as dichloromethane or chloroform in a suitable base such as pyridine or triethylamine at room temperature for 1-2 h, to yield the compound of formula 208 (Reaction 21e).

Alternately, the compound of formula 206 is reacted with the compound of formula 8 (v) in a suitable solvent such as toluene and a coupling agent such as trimethylaluminium to yield the compound of formula 208.

### Step 6

### Preparation of Compound of Formula 209:

The compound of formula 206 is reacted with commercially available compound of formula 8 (vi) in a suitable solvent such as dichloromethane or chloroform in a suitable base such as pyridine or triethylamine at room temperature for 1-2 h, to yield the compound of formula 209 (Reaction 21f).

### Scheme 22:

Scheme 22 depicts a process for the preparation of the compounds of formula 1 (referred in Scheme 22 as compound 216 (L is *NHC(O)NH), compound 217 (L is *C(O)NH) and compound 218 (L is *SO₂NH);

wherein Z is:

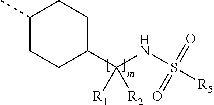

A, n, R₁, R₂ and R₅ are as defined in formula 1). Said process includes steps 1 to 8 as described below:

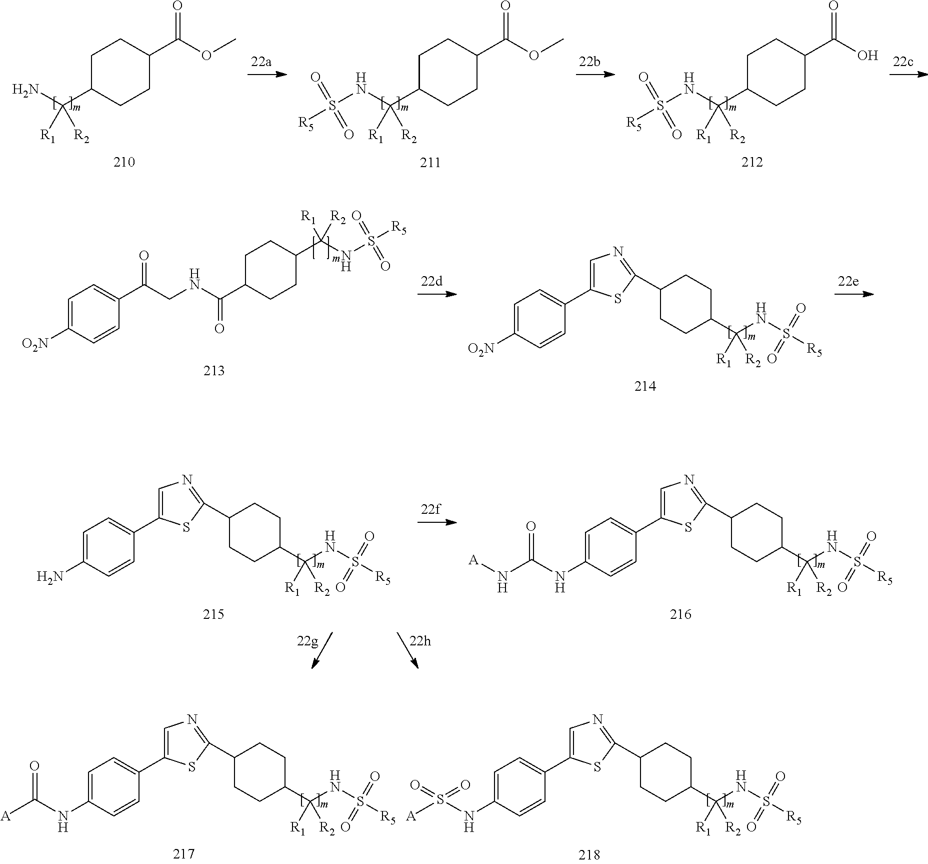

### Step 1

### Preparation of Compound of Formula 211:

Commercially available compound of formula 210:

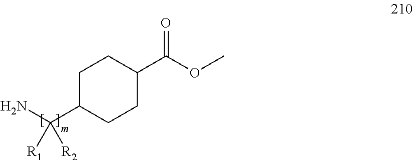

wherein R₁, R₂ and n are as defined in formula 1;
is reacted with a reagent such as triflic anhydride in presence of a base such as DIPEA in a suitable solvent such as dichloromethane at room temperature for 16 h to yield compound of formula 211 (Reaction 22a).

### Step 2

### Preparation of Compound of Formula 212:

The compound of formula 211 is hydrolysed using LiOH in a suitable solvent such as THF at room temperature for 16 h to yield the compound of formula 212 (Reaction 22b).

### Step 3

### Preparation of Compound of Formula 213:

The compound of formula 212 is reacted with 2-amino-(4-nitro)acetophenone hydrochloride and the reagent, HATU in presence of a base such as triethyl amine in a suitable solvent such as DMF at room temperature for 3-5 h to yield the compound of formula 213 (Reaction 22c).

### Step 4

### Preparation of Compound of Formula 214:

The compound of formula 213 is refluxed with a reagent such as Lawesson's reagent in a suitable solvent such as 1,4-dioxane or THF, at a temperature range of 60° C. to 110° C., to yield the compound of formula 214 (Reaction 22d).

### Step 5

### Preparation of Compound of Formula 215:

The compound of formula 214 is reduced with a suitable reducing agent such as Fe and NH₄Cl in a suitable solvent mixture of EtOH, THF and water at a temperature range of 70° C. to 80° C. for 2-6 h to yield compound of formula 215 (Reaction 22e).

### Step 6

### Preparation of Compound of Formula 216:

The compound of formula 215 is reacted with commercially available compound of formula 8 (i) in a suitable solvent such as THF or dichloromethane at room temperature for 2-16 h to yield the compound of formula 216 (Reaction 22f).

Alternately, the compound of formula 215 is reacted with the compound of formula 8 (ii) in presence of a coupling agent such as carbonyl diimidazole in a suitable solvent such as THF at room temperature for about 24 h to yield the compound of formula 216.

### Step 7

### Preparation of Compound of Formula 217:

The compound of formula 215 is reacted with commercially available compound of formula 8 (iv) in a suitable solvent such as dichloromethane or chloroform in a suitable base such as pyridine at room temperature for 1-2 h, to yield the compound of formula 217 (Reaction 22g).

Alternately, the compound of formula 216 is reacted with commercially available compound of formula 8 (v) in a suitable solvent such as toluene and a coupling agent such as trimethylaluminium to yield the compound of formula 217.

### Step 8

### Preparation of Compound of Formula 218:

The compound of formula 215 is reacted with commercially available compound of formula 8 (vi) in a suitable solvent such as dichloromethane or chloroform in a suitable base such as pyridine at room temperature for 1-2 h, to yield the compound of formula 218 (Reaction 22h).

### Scheme 23:

Scheme 23 depicts a process for the preparation of the compounds of formula 1 (referred in Scheme 23 as compound 224 (R₃ is (C₁-C₁₂ alkyl)) and compound 225 (R₃ is H);

wherein Z is:

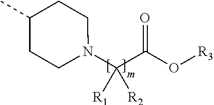

### B is

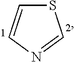

wherein 1 and 2 are the points of attachment of B to phenyl and to Z respectively; L is *NHC(O)NH; A, m, R₁, R₂ and R₅ are as defined in formula 1). Said process includes steps 1 to 7 as described below:

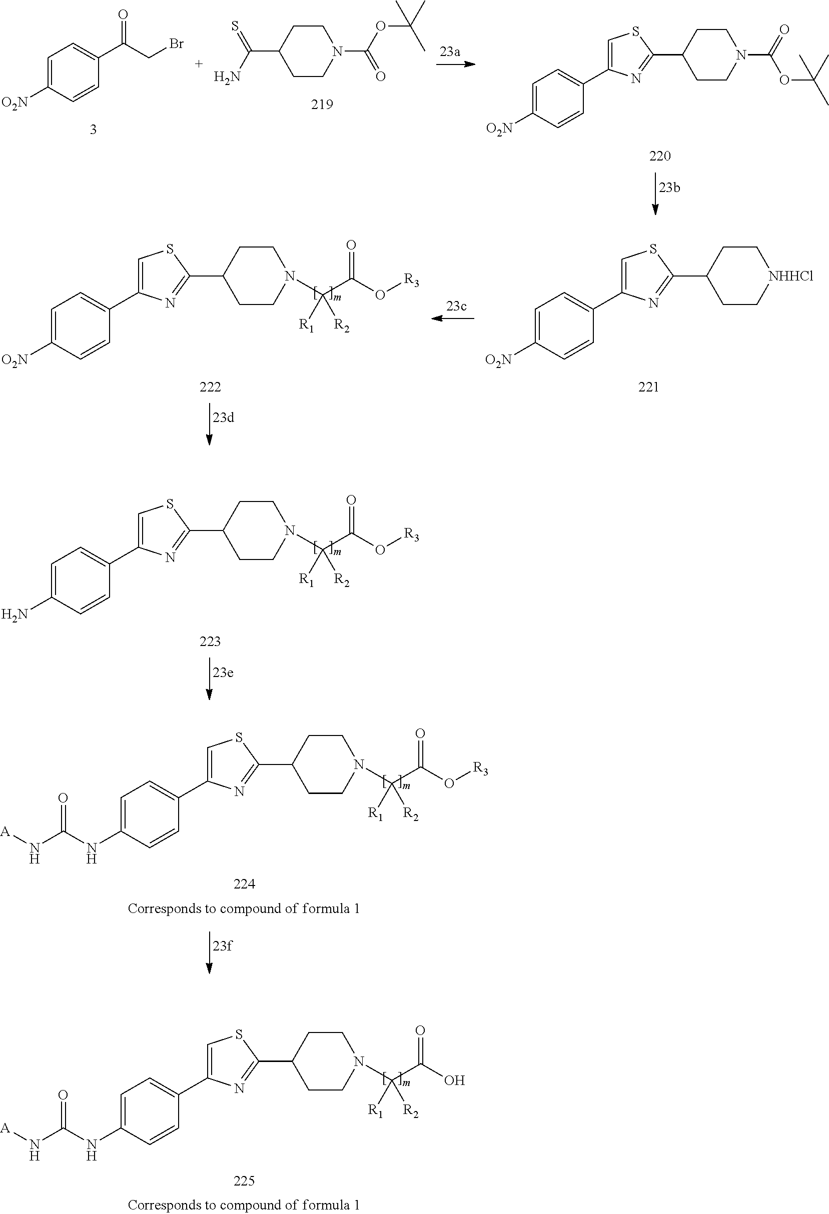

### Step 1

### Preparation of Compound of Formula 220:

The compound of formula 3 is refluxed with compound of formula 219 at a temperature range of 75° C. to 85° C. for 3-5 h to yield the compound of formula 220 (Reaction 23a).

### Step 2

### Preparation of Compound of Formula 221:

The compound of formula 220 is treated with 1N HCl in a suitable solvent such as ethyl acetate at room temperature to yield the compound of formula 221 (Reaction 23b).

### Step 3

### Preparation of Compound of Formula 222:

The compound of formula 221 is reacted with the commercially available reagent:

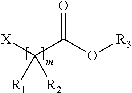

wherein X is halogen; m, R₁, R₂ and R₃ are as defined in formula 1;
in presence of a base such as triethylamine in a suitable solvent such as toluene at a temperature range of 100° C. to 120° C. to yield the compound of formula 222 (Reaction 23c).

### Step 4

### Preparation of Compound of Formula 223:

The compound of formula 222 is reduced with a reducing agent such as Fe and NH₄Cl in a suitable solvent mixture of EtOH, THF and water at a temperature range of 70° C. to 80° C. for 2-6 h, to yield compound of formula 223 (Reaction 23d).

### Step 5

### Preparation of Compound of Formula 224:

The compound of formula 223 is reacted with a compound of formula 8 (i) in a suitable solvent such as THF or dichloromethane at room temperature for 2-16 h, to yield the compound of formula 224 (Reaction 23e).

Alternately, the compound of formula 223 is reacted with the compound of formula 8 (ii) in presence of a coupling agent such as carbonyl diimidazole in a suitable solvent such as THF at room temperature for 24 h to yield the compound of formula 224.

### Step 6

### Preparation of Compound of Formula 225:

The compound of formula 224 is hydrolysed using suitable reagent such as aqueous LiOH in a suitable solvent such as THF or methanol or a mixture thereof, at room temperature for 2-16 h at room temperature, to yield the compound of formula 225 (Reaction 23f).

### Step 7

The carboxylic acid (compound of formula 225) is optionally converted to its corresponding ester prodrugs by any suitable method well known in the art.

### Scheme 24:

Scheme 24 depicts a process for the preparation of the compounds of formula 1 (referred in Scheme 24 as compound 230 (R₃ is t-butyl; m=0), compound 231 (R₃ is H; m=0), compound 235 (R₃ is (C₁-C₁₂ alkyl)) and compound 236 (R₃ is H); wherein Z is:

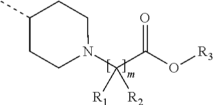

### B is

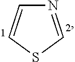

wherein 1 and 2 are the points of attachment of B to phenyl and to Z respectively; L is *NHC(O)NH; A, m, R₁, R₂ and R₃ are as defined in formula 1). Said process includes steps 1 to 11 as described below:

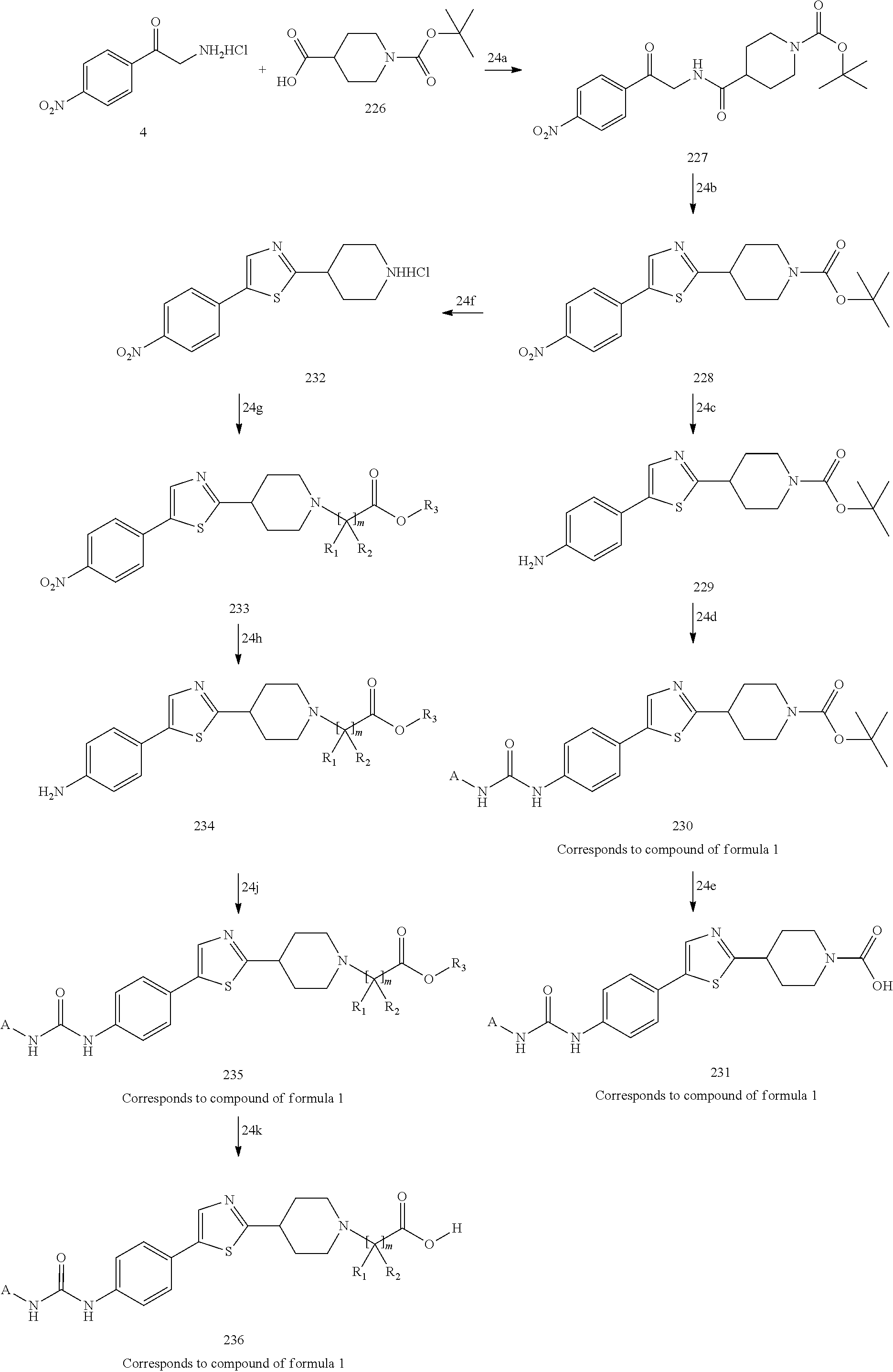

### Step 1

### Preparation of Compound of Formula 227:

The compound of formula 4 is reacted with commercially available compound of formula 226 in presence of a base such as DIPEA in a suitable solvent such as DMF in presence of HATU at room temperature for 30 min to 1 h to yield the compound of formula 227 (Reaction 24a).

### Step 2

### Preparation of Compound of Formula 228:

The compound of formula 227 is reacted with Lawesson's reagent in a suitable solvent such as dioxane at 50° C. to 70° C. for 2-4 h to yield the compound of formula 228 (Reaction 24b).

### Step 3

### Preparation of Compound of Formula 229:

The compound of formula 228 is reduced with a reducing agent such as Fe and NH₄Cl in a suitable solvent mixture of EtOH, THF and water at a temperature range of 70° C. to 80° C. for 2-6 h, to yield compound of formula 229 (Reaction 24c).

### Step 4

### Preparation of Compound of Formula 230:

The compound of formula 229 is reacted with a compound of formula 8 (i) in a suitable solvent such as THF or dichloromethane at room temperature for 2-16 h, to yield the compound of formula 230 (Reaction 24d).

Alternately, the compound of formula 229 is reacted with the compound of formula 8 (ii) in presence of a coupling agent such as carbonyl diimidazole in a suitable solvent such as THF at room temperature for 24 h to yield the compound of formula 230.

### Step 5

### Preparation of Compound of Formula 231:

The compound of formula 230 is hydrolysed using suitable reagent such as aqueous LiOH in a suitable solvent such as THF or methanol or a mixture thereof, at room temperature for 2-16 h at room temperature, to yield the compound of formula 231 (Reaction 24e).

### Step 6

### Preparation of Compound of Formula 232:

The compound of formula 228 is treated with 1N HCl in a suitable solvent such as ethyl acetate at room temperature to yield the compound of formula 232 (Reaction 24f).

### Step 7

### Preparation of Compound of Formula 233:

The compound of formula 232 is reacted with the commercially available reagent:

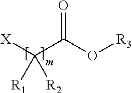

wherein X is halogen; m, R₁, R₂ and R₃ are as defined in formula 1;
in presence of a base such as triethylamine in a suitable solvent such as toluene at a temperature range of 100° C. to 120° C. to yield the compound of formula 233 (Reaction 24g).

### Step 8

### Preparation of Compound of Formula 234:

The compound of formula 233 is reduced with a reducing agent such as Fe and NH₄Cl in a suitable solvent mixture of EtOH, THF and water at a temperature range of 70° C. to 80° C. for 2-6 h, to yield compound of formula 234 (Reaction 24h).

### Step 9

### Preparation of Compound of Formula 235:

The compound of formula 234 is reacted with a compound of formula 8 (i) in a suitable solvent such as THF or dichloromethane at room temperature for 2-16 h, to yield the compound of formula 235 (Reaction 24j).

Alternately, the compound of formula 234 is reacted with the compound of formula 8 (ii) in presence of a coupling agent such as carbonyl diimidazole in a suitable solvent such as THF at room temperature for 24 h to yield the compound of formula 235.

### Step 10

### Preparation of Compound of Formula 236:

The compound of formula 235 is hydrolysed using suitable reagent such as aqueous LiOH in a suitable solvent such as THF or methanol or a mixture thereof, at room temperature for 2-16 h at room temperature, to yield the compound of formula 236 (Reaction 24k).

### Step 11

The carboxylic acid (compound of formula 231 and 236) is optionally converted to its corresponding ester prodrugs by any suitable method well known in the art.

### Scheme 25:

Scheme 25 depicts a process for the preparation of the compounds of formula 1 (referred in Scheme 25 as compound 241 and compound 244;

wherein Z is:

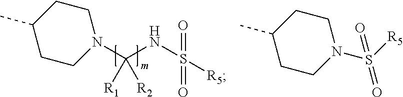

### B is

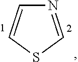

wherein 1 and 2 are the points of attachment of B to phenyl and to Z respectively; L is *NHC(O)NH; A, m, R₁, R₂ and R₅ are as defined in formula 1). Said process includes steps 1 to 8 as described below:

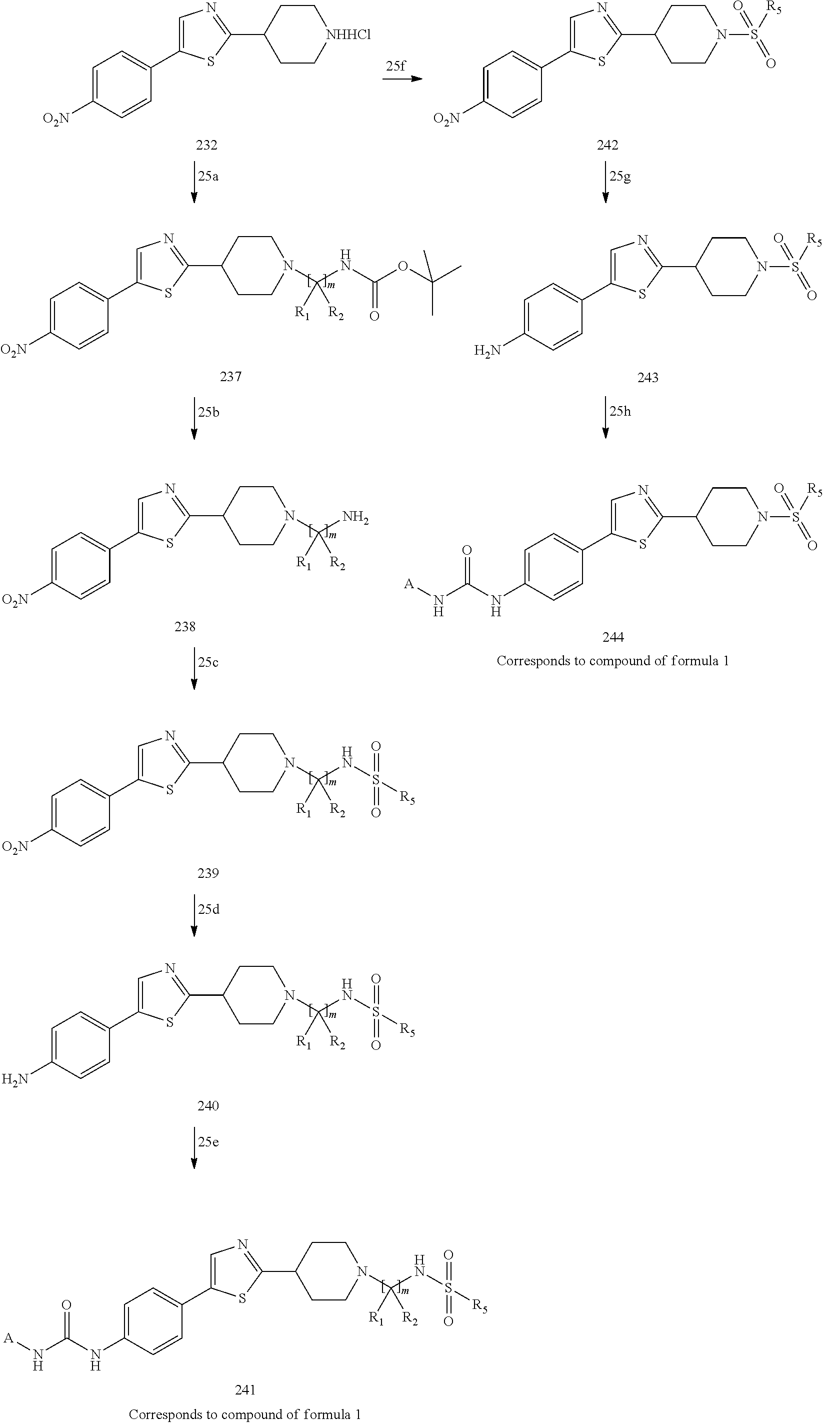

### Step 1

### Preparation of Compound of Formula 237:

The compound of formula 232 is reacted with t-butyl 2-bromoethylcarbamate in presence of a base such as K₂CO₃ in a suitable solvent such as DMF at a temperature range of 50° C. to 80° C. for 2-4 h to yield the compound of formula 237 (Reaction 25a).

### Step 2

### Preparation of Compound of Formula 238:

The compound of formula 237 is reacted with HCl in a suitable solvent such as isopropanol or methanol at room temperature for 12-15 h to yield the compound of formula 238 (Reaction 25b).

### Step 3

### Preparation of Compound of Formula 239:

The compound of formula 238 is reacted with triflic anhydride in a suitable solvent such as dichloromethane and a suitable base such as triethylamine at room temperature for 10-16 h to yield the compound of formula 239 (Reaction 25c).

### Step 4

### Preparation of Compound of Formula 240:

The compound of formula 239 is reduced with a reducing agent such as Fe and NH₄Cl in a suitable solvent mixture of EtOH, THF and water at a temperature range of 70° C. to 80° C. for 2-6 h, to yield compound of formula 240 (Reaction 25d).

### Step 5

### Preparation of Compound of Formula 241:

The compound of formula 240 is reacted with a compound of formula 8 (i) in a suitable solvent such as THF or dichloromethane at room temperature for 2-16 h, to yield the compound of formula 241 (Reaction 25e).

Alternately, the compound of formula 240 is reacted with the compound of formula 8 (ii) in presence of a coupling agent such as carbonyl diimidazole in a suitable solvent such as THF at room temperature for 24 h to yield the compound of formula 241.

### Step 6

### Preparation of Compound of Formula 242:

The compound of formula 232 is reacted with the commercially available reagent:

R₅SO₂Cl or R₅(SO₂)₂O;

wherein R₅ is as defined in formula 1; in presence of a base such as triethylamine in a suitable solvent such as dichloromethane at room temperature for 16 h to yield the compound of formula 242 (Reaction 25f).

### Step 7

### Preparation of Compound of Formula 243:

The compound of formula 242 is reduced with a reducing agent such as Fe and NH₄Cl in a suitable solvent mixture of EtOH, THF and water at a temperature range of 70° C. to 80° C. for 2-6 h, to yield compound of formula 243 (Reaction 25g).

### Step 8

### Preparation of Compound of Formula 244:

The compound of formula 243 is reacted with a compound of formula 8 (i) in a suitable solvent such as THF or dichloromethane at room temperature for 2-16 h, to yield the compound of formula 244 (Reaction 25h).

Alternately, the compound of formula 243 is reacted with the compound of formula 8 (ii) in presence of a coupling agent such as carbonyl diimidazole in a suitable solvent such as THF at room temperature for 24 h to yield the compound of formula 244.

### Scheme 26:

Scheme 26 depicts a process for the preparation of the compounds of formula 1 (referred in Scheme 26 as compound 250 and compound 251;

wherein Z is:

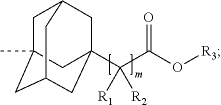

### B is

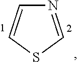

wherein 1 and 2 are the points of attachment of B to phenyl and to Z respectively; L is *NHC(O)NH; A, m, R₁, R₂ and R₃ are as defined in formula 1). Said process includes steps 1 to 7 as described below:

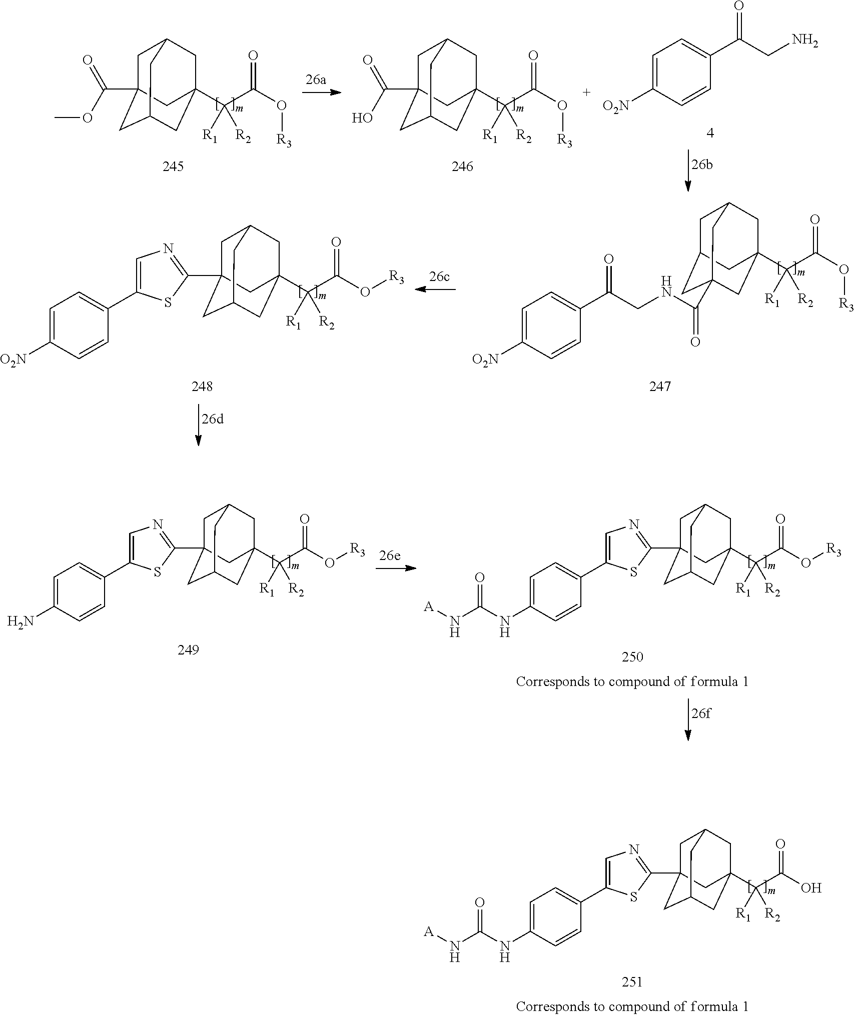

### Step 1

### Preparation of Compound of Formula 246:

Commercially available compound of formula 245 is treated with a base such as KOH in a suitable solvent such as methanol at a temperature range of 60° C. to 80° C. for 16 h followed by acidification with an inorganic acid such as dilute HCl to yield the compound of formula 246 (Reaction 26a).

### Step 2

### Preparation of Compound of Formula 247:

The compound of formula 246 is reacted with the compound of formula 4 in presence of the reagent, HATU and a base such as DIPEA in a suitable solvent such as DMF at room temperature for 30 min to 2 h to yield the compound of formula 247 (Reaction 26b).

### Step 3

### Preparation of Compound of Formula 248:

The compound of formula 247 is reacted with Lawesson's reagent in a suitable solvent such as dioxane at 50° C. to 70° C. for 2-4 h to yield the compound of formula 248 (Reaction 26c).

### Step 4

### Preparation of Compound of Formula 249:

The compound of formula 248 is reduced with a reducing agent such as Fe and NH₄Cl in a suitable solvent mixture of EtOH, THF and water at a temperature range of 70° C. to 80° C. for 2-6 h, to yield compound of formula 249 (Reaction 26d).

### Step 5

### Preparation of Compound of Formula 250:

The compound of formula 249 is reacted with a compound of formula 8 (i) in a suitable solvent such as THF or dichloromethane at room temperature for 2-16 h, to yield the compound of formula 250 (Reaction 26e).

Alternately, the compound of formula 249 is reacted with the compound of formula 8 (ii) in presence of a coupling agent such as carbonyl diimidazole in a suitable solvent such as THF at room temperature for 24 h to yield the compound of formula 250.

### Step 6

### Preparation of Compound of Formula 251:

The compound of formula 250 is hydrolysed using suitable reagent such as aqueous LiOH in a suitable solvent such as THF or methanol or a mixture thereof, at room temperature for 2-16 h at room temperature, to yield the compound of formula 251 (Reaction 26f).

### Step 7

The carboxylic acid (compound of formula 251) is optionally converted to its corresponding ester prodrugs by any suitable method well known in the art.

### Scheme 27:

Scheme 27 depicts a process for the preparation of the compounds of formula 1; (referred in Scheme 27 as compound 13 (R₃ is (C₁-C₁₂)-alkyl) and compound 14 (R₃ is H);

wherein Z is:

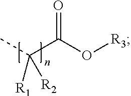

### B is

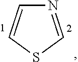

wherein 1 and 2 are the points of attachment of B to phenyl and to Z respectively; L is *NHC(O); A is a (C₃-C₇)-membered cyclic ring containing N and optionally other heteroatoms such as O, N and S; n, R₁, R₂ and R₃ are as defined in formula 1). Said process includes steps 1 and 2 as described below:

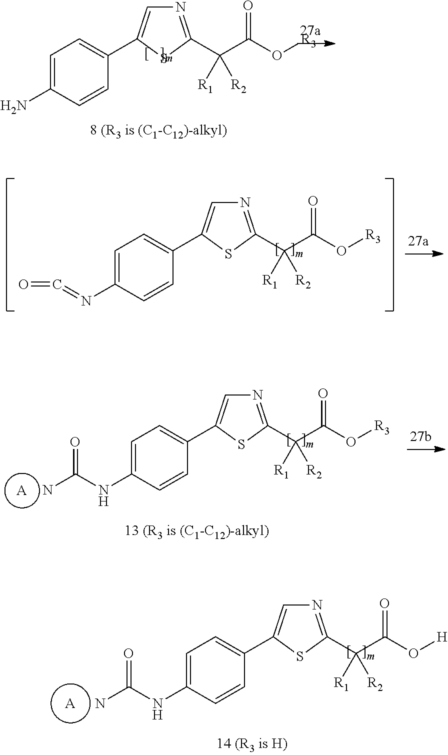

### Step 1a

### Preparation of Compound of Formula 9 (R₃ is (C₁-C₁₂)-Alkyl)

The compound of formula 8 (R₃ is (C₁-C₁₂)-alkyl) is reacted with triphosgene in presence of a suitable base such as triethylamine in a suitable solvent such as dichloromethane at room temperature for 1-2 h, followed by addition of the reagent:

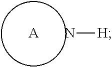

wherein A is a (C₃-C₇)-membered cyclic ring containing N and optionally other heteroatoms such as O, N and S; A-NH₂ or NH for 16-24 h to yield the compound of formula 13 (R₃ is (C₁-C₁₂)-alkyl)(Reaction 27a); and

### Step 1b

### Preparation of Compound of Formula 10 (R₃ is H)

The compound of formula 9 (R₃ is (C₁-C₁₂)-alkyl) is hydrolysed using suitable reagent such as aqueous LiOH in a suitable solvent such as THF or methanol or a mixture thereof, at room temperature for 2-16 h at room temperature, to yield the compound of formula 14 (R₃ is H) (Reaction 27b); and

### Step 2

The carboxylic acid (compound of formula 10) is optionally converted to its corresponding ester prodrugs by any suitable method well known in the art.

In all the above mentioned schemes 1-27, the carboxylic acids formed may be optionally converted to their pharmaceutically acceptable salts. In one aspect, the carboxylic acids of formula 1 of the present invention are converted to their sodium or potassium salts.

The present invention also includes within its scope all isotopically labeled forms of compounds of formula 1, wherein one or more atoms of compounds of formula 1 are replaced by their respective isotopes. Examples of isotopes that may be incorporated into the compounds disclosed herein include, but are not limited to, isotopes of hydrogen such as ²H and ³H, carbon such as ¹¹C, ¹³C, and ¹⁴C, nitrogen such as ¹³N and ¹⁵N, oxygen such as ¹⁵O, ¹⁷O and ¹⁸O, chlorine such as ³⁶O, fluorine such as ¹⁸F and sulphur such as ³⁵S.

Substitution with heavier isotopes, for example, replacing one or more key carbon-hydrogen bonds with carbon-deuterium bond may show certain therapeutic advantages, for example, longer metabolism cycles, improved safety or greater effectiveness.

Isotopically labeled forms of compounds of formula 1 can be prepared by conventional techniques known to those skilled in the art or by processes analogous to those described above and in the subsequent section on examples by using an appropriate isotopically labeled reagent instead of non-labeled reagent.

The compounds of the present invention can also be converted into their corresponding pharmaceutically acceptable salts or solvates. The pharmaceutically acceptable salts of the compounds of the present invention are in particular salts, which can be used physiologically.

The term “pharmaceutically acceptable salts” is meant to include salts of the active compounds which are prepared with acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, magnesium, ammonium or organic base salt, or a similar salt. Examples of pharmaceutically acceptable organic base addition salts include those derived from organic bases like lysine, arginine, guanidine, diethanolamine and the like.

When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, oxalic, maleic, malonic, benzoic, succinic, suberic, fumaric, mandelic, phthalic, benzenesulfonic, p-tolylsulfonic, citric, tartaric, methanesulfonic, glucuronic or galacturonic acids and the like. Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties. An example of physical properties that may differ is solubility in polar solvents.

Certain compounds of the present invention can exist in unsolvated forms as well as solvated forms, including hydrated forms. Certain compounds of the present invention may exist in multiple crystalline or amorphous forms. In general, all physical forms are equivalent for the uses contemplated by the present invention and are intended to be within the scope of the present invention.

Various polymorphs of compounds of formula 1 can be prepared by crystallization of the compounds under different conditions. The different conditions are, for example, using different commonly used solvents or their mixtures for crystallization; crystallization at different temperatures; various modes of cooling, ranging from very fast to very slow cooling during crystallizations. Polymorphs can also be obtained by heating or melting the compound followed by gradual or fast cooling. The presence of polymorphs can be determined by IR (Infra-red) spectroscopy, solid probe NMR (Nuclear Magnetic Resonance) spectroscopy, differential scanning calorimetry, powder X-ray diffraction or such other techniques.

Those skilled in the art will recognize that stereocentres exist in compounds of formula 1. Accordingly, the present invention includes all possible stereoisomers and geometric isomers of formula 1 and includes not only racemic compounds but also the optically active isomers as well. When a compound of formula 1 is desired as a single enantiomer, it may be obtained either by resolution of the final product or by stereospecific synthesis from either isomerically pure starting material or any convenient intermediate. Resolution of the final product, an intermediate or a starting material may be effected by any suitable method known in the art for example Chiral reagents for Asymmetric Synthesis by Leo A. Paquette; John Wiley & Sons Ltd. Additionally, in situations wherein tautomers of the compounds of formula 1 are possible, the present invention is intended to include all tautomeric forms of the compounds.

The present invention also envisages prodrugs of the compound of formula 1. Prodrug derivatives of any compound of the invention are derivatives of said compounds which following administration release the parent compound in vivo via some chemical or physiological process, e.g., a prodrug on being brought to the physiological pH or through enzyme action is converted to the parent compound. Preferred are pharmaceutically acceptable ester derivatives convertible by solvolysis under physiological conditions to the parent carboxylic acid, e.g., lower alkyl esters, cycloalkyl esters, lower alkenyl esters, benzyl esters, mono- or di-substituted lower alkyl esters such as the pivaloyloxymethyl ester and the like conventionally used in the art (An introduction to Medicinal Chemistry, Graham. L. Patrick, Second Edition, Oxford University Press, pg 239-248; Prodrugs: Challenges and Rewards, Part 1 and Part 2, AAPS Press, Edited by Valentino J. Stella, Renald T. Borchardt, Michael J. Hagemon, Reza Oliyai, Hans Maag, Jefferson W. Tilley).

The present invention furthermore relates to pharmaceutical compositions that contain an effective amount of at least one compound of formula 1 or its physiologically tolerable salt in addition to a customary pharmaceutically acceptable carrier, and to a process for the production of a pharmaceutical composition, which includes bringing at least one compound of formula 1, into a suitable administration form using a pharmaceutically suitable and physiologically tolerable excipient and, if appropriate, further suitable active compounds, additives or auxiliaries.

As used herein, the term “pharmaceutically acceptable carrier” refers to a material that is non-toxic, inert, solid, semi-solid or liquid filler, diluent, encapsulating material or formulation auxiliary of any type which is compatible with a subject, preferably a mammal, more preferably a human, and is suitable for delivering an active agent to the target site without terminating the activity of the agent.

The present invention also envisages the use of a compound of formula 1 or a pharmaceutically acceptable salt of the compound in combination with other pharmaceutically active compounds. For instance, a pharmaceutical composition including a compound of formula 1 or a pharmaceutically acceptable salt can be administered to a mammal, in particular a human, with an anti-diabetic agent or an anti-obesity agent, in mixtures with one another or in the form of pharmaceutical preparations.

The term, “therapeutically effective amount” as used herein means an amount of compound or composition comprising compound of formula 1, effective in producing the desired therapeutic response in a particular patient suffering from DGAT1 mediated disorders. The therapeutically effective amount of the compound or composition will vary with the particular condition being treated, the age and physical condition of the end user, the severity of the condition being treated/prevented, the duration of the treatment, the nature of concurrent therapy, the specific compound or composition employed, the particular pharmaceutically acceptable carrier utilized, and like factors.

The term “subject” as used herein refers to an animal, preferably a mammal, and most preferably a human.

The term “mammal” used herein refers to warm-blooded vertebrate animals of the class Mammalia, including humans, characterized by a covering of hair on the skin and, in the female, milk-producing mammary glands for nourishing the young. The term mammal includes animals such as cat, dog, rabbit, bear, fox, wolf, monkey, deer, mouse, pig as well as human.

As used herein, the terms “treatment” “treat” and “therapy” and the like refer to alleviate, slow the progression, prophylaxis, attenuation or cure of existing disease (e.g., diabetes). “Prevent”, as used herein, refers to delaying, slowing, inhibiting, reducing or ameliorating the onset of diseases or disorders mediated by diacylglycerol acyltransferase (DGAT), particularly DGAT1.

In one aspect, the compound used for the manufacture of the medicament for treating DGAT1 mediated disorder is one of those as defined herein, especially the herein specifically described compounds.

Among the preferred DGAT especially DGAT1 mediated disorders, the following can be mentioned: obesity, diabetes mellitus, impaired glucose tolerance, diabetic neuropathy, diabetic nephropathy, diabetic retinopathy, anorexia nervosa, bulimia, cachexia, syndrome X, insulin resistance, hypoglycemia, hyperglycemia, hyperuricemia, hyperinsulinemia, hypercholesterolemia, hyperlipidemia, dyslipidemia, mixed dyslipidemia, hypertriglyceridemia, pancreatitis, metabolic acidosis, ketosis, steatosis, dysmetabolic syndrome and nonalcoholic fatty liver disease, skin disorders, acne, cardiovascular diseases such as atherosclerosis, arteriosclerosis, acute heart failure, congestive heart failure, coronary artery disease, cardiomyopathy, myocardial ischaemia, myocardial infarction, angina pectoris, hypertension, hypotension, stroke, ischemia, ischemic reperfusion injury, aneurysm, restenosis, peripheral vascular disease and vascular stenosis, diseases of skin such as acne, infertility, polycystic ovary syndrome and Hepatitis C infection.

In another aspect, the DGAT1 associated disorder is selected from impaired glucose tolerance, diabetes mellitus, insulin resistance, diabetic neuropathy, diabetic nephropathy, diabetic retinopathy, hypercholesterolemia, hypertriglyceridemia, hyperlipidemia and obesity.

In yet another aspect, the present invention provides a method for the treatment of diseases or disorders mediated by DGAT1, comprising administering to a mammal in need thereof a therapeutically effective amount of a compound of formula 1, or a pharmaceutically acceptable salt or prodrug thereof.

In a further aspect, the present invention provides a method for the treatment of obesity comprising administering to a mammal in need thereof a therapeutically effective amount of a compound of formula 1, or a pharmaceutically acceptable salt or prodrug thereof.

In a still further aspect, the present invention provides the use of a compound of formula 1 in the treatment of diseases or disorders mediated by DGAT1.

In another aspect, the present invention provides the use of a compound of formula 1 in the treatment of obesity.

In an aspect, the present invention provides the use of a compound of formula 1 or a pharmaceutically acceptable salt or a produg thereof, for the manufacture of a medicament for the treatment of diseases or disorders mediated by DGAT1.

According to another aspect of the present invention, there is provided the use of a compound of formula 1 or a pharmaceutically acceptable salt or a prodrug thereof, for the manufacture of a medicament for the treatment of obesity.

In a further aspect, the methods for treating DGAT1 associated disorders described herein use the pharmaceutical compositions described above can be administered by the following administration routes, modes, etc.

### Pharmaceutical Compositions and Methods

The pharmaceuticals can be administered orally, for example in the form of pills, tablets, coated tablets, capsules, granules or elixirs. Administration, however, can also be carried out rectally, for example in the form of suppositories, or parenterally, for example intravenously, intramuscularly or subcutaneously, in the form of injectable sterile solutions or suspensions, or topically, for example in the form of solutions or transdermal patches, or in other ways, for example in the form of aerosols or nasal sprays.

As used herein, the term “pharmaceutically acceptable” means that the carrier, diluent, excipients, and/or salt must be compatible with the other ingredients of the formulation, and not deleterious to the recipient thereof.

The pharmaceutical preparations according to the invention are prepared in a manner known and familiar to one skilled in the art. Pharmaceutically acceptable inert inorganic and/or organic carriers and/or additives can be used in addition to the compound(s) of formula 1, and/or its (their) physiologically tolerable salt(s). For the production of pills, tablets, coated tablets and hard gelatin capsules it is possible to use, for example, lactose, corn starch or derivatives thereof, gum arabica, magnesia or glucose, etc. Carriers for soft gelatin capsules and suppositories are, for example, fats, waxes, natural or hardened oils, etc. Suitable carriers for the production of solutions, for example injection solutions, or of emulsions or syrups are, for example, water, physiological sodium chloride solution or alcohols, for example, ethanol, propanol or glycerol, sugar solutions, such as glucose solutions or mannitol solutions, or a mixture of the various solvents which have been mentioned.

The pharmaceutical preparations normally contain about 1 to 99%, for example, about 5 to 70%, or from about 10 to about 30% by weight of the compound of the formula 1 or its physiologically tolerable salt. The amount of the compound of the formula 1 or its physiologically tolerable salt in the pharmaceutical preparations normally is from about 5 to 500 mg. The dose of the compounds of this invention, which is to be administered, can cover a wide range. The dose to be administered daily is to be selected to suit the desired effect. A suitable dosage is about 0.001 to 100 mg/kg/day of the compound of formula 1 or their physiologically tolerable salt, for example, about 0.01 to 50 mg/kg/day of a compound of formula 1 or a pharmaceutically acceptable salt of the compound. If required, higher or lower daily doses can also be administered.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compounds employed, the age, sex, weight, condition, general health and prior medical history of the patient being treated, and like factors well known in the medical arts.

In addition to the compound of the formula 1 or its physiologically acceptable salt and carrier substances, the pharmaceutical preparations can contain additives such as, for example, fillers, antioxidants, dispersants, emulsifiers, defoamers, flavors, preservatives, solubilizers or colorants. They can also contain two or more compounds of formula 1 or their physiologically tolerable salts. Furthermore, in addition to at least one compound of formula 1 or its physiologically tolerable salt, the pharmaceutical preparations can also contain one or more other therapeutically or prophylactically active ingredients.

It is understood that modifications that do not substantially affect the activity of the various aspects of this invention are included within the invention disclosed herein. Accordingly, the following examples are intended to illustrate but not to limit the present invention.

The following abbreviations or terms are used herein:
AlCl₃: Aluminium chloride
BOC: ter-Butyloxycarbonyl
BOP: Benzotriazol-1-yloxy)tris(dimethylamino)phosphonium hexafluorophosphate
CDCl₃: Deuteriated chloroform
CDI: Carbonyldiimidazole
CHCl₃: Chloroform
DBU: 1,8-Diazabicyclo[5.4.0]undec-7-ene
DCE: Dichloroethane
DCM: Dichloromethane
DIPEA: N,N-Diisopropylethylamine
DMF: N,N-Dimethylformamide
DMF-DMA: N,N-Dimethylformamide dimethyl acetal
DMSO: Dimethylsulfoxide
DMSO-d₆: Deuteriated dimethylsulfoxide
EtOAc: Ethyl acetate
EtOH: Ethanol
g: gram
h: hour(s)
HCl: Hydrochloric acid
HATU: 2-(7-Aza-1H-benzotriazole-1-yl)-1,1,3,3-tetramethyluronium hexafluorophosphate
H₂SO₄: Sulfuric acid
H₂O: Water
HgO: Mercury Oxide
KOH: Potassium hydroxide
K₂CO₃: Potassium carbonate
LiOH: Lithium hydroxide
MeOH: Methanol
mg: milligram(s)
mL: milliliter
min: minute(s)
NaH: Sodium hydride
NaOH: Sodium hydroxide
NaHCO₃: Sodium bicarbonate
Na₂CO₃: Sodium carbonate
Na₂SO₄: Sodium sulfate
NH₄Cl: Ammonium chloride
Pd/C: Palladium over activated carbon
POCl₃: Phosphoryl chloride
Room temperature: 20° C.-35° C.
TEA: Triethylamine
THF: Tetrahydrofuran
° C.: Degree Celsius

### Example 1

### 2-Bromo-1-(4-nitrophenyl)ethanone

4-Nitroacetophenone (25g) in ether (250 mL) was treated with aluminium chloride (catalytic amount) followed by bromine (7.77 mL) over 10 min and the reaction was stirred for 30 min. The reaction was quenched with aqueous sodium bicarbonate, the ether layer was separated, dried over anhydrous Na₂SO₄ and concentrated to yield a residue. The residue obtained was crystallized using ethyl acetate and petroleum ether to afford the title compound (according to the procedure described in U.S. Pat. No. 4,812,470). Yield: 25.5 g (69° A)); ¹H NMR (CDCl₃, 300 MHz): δ 8.19 (d, 2H), 8.36 (d, 2H), 4.47 (s, 2H).

### Example 2

### 2-Amino-1-(4-nitrophenyl)ethanone hydrochloride

The compound of example 1 (25g) was dissolved in dichloromethane (250 mL), hexamethylenetetramine (20.1 g) was added and the mixture was stirred for 1 h. The reaction was filtered to yield a crude residue (30g), which was stirred in a mixture of ethanol (162 mL) and concentrated HCl (40 mL) for about 3 h. On allowing to stand for about 48 h, a solid separated out, which was filtered, washed with water and dried to afford the title compound (according to the procedure described in U.S. Pat. No. 4,812,470). Yield: 11.8 g (72%); ¹H NMR (DMSO-d₆, 300 MHz): δ 8.3 (bs, 3H), 8.38 (d, 2H), 8.27 (d, 2H), 4.68 (s, 2H).

### Example 3

### Methyl 4-(2-(4-nitrophenyl)-2-oxoethylamino)-4-oxobutanoate

The compound of example 2 (17.5 g) was dissolved in ethyl acetate (180 mL), to which triethylamine (12.53 mL) was added. To this reaction mixture, methyl 4-chloro-4-oxobutanoate (11 mL) in ethyl acetate (70 mL) was added dropwise and the reaction mixture was refluxed for 2 h. The reaction mixture was cooled, water was added and the reaction mixture was extracted with ethyl acetate. The organic layer was dried over anhydrous Na₂SO₄ and concentrated to yield a crude residue, which was purified by column chromatography (silicagel, 30% ethyl acetate in petroleum ether to obtain a solid. The solid was crystallized using ethyl acetate in petroleum ether to afford the title compound. Yield: 8.8 g (37%); ¹H NMR (DMSO-d₆, 300 MHz): δ 8.37 (d, 2H), 8.15 (d, 2H), 6.64 (t, 1H), 4.82 (d, 2H), 3.71 (s, 3H), 2.72 (t, 2H), 2.64 (t, 2H); MS: m/z 295 (M+1).

### Example 4

### Methyl 3-(5-(4-nitrophenyl)thiazol-2-yl)propanoate

The compound of example 3 (8.7 g) was dissolved in 1,4-dioxane (174 mL) to which Lawesson's reagent (11.97 g) was added and the reaction mixture was heated to reflux for 2 h. The reaction mixture was cooled, water was added and the reaction mixture was neutralized with a saturated solution of sodium carbonate. Ethyl acetate was added and the organic layer was separated and dried over anhydrous Na₂SO₄. The organic layer was concentrated to yield a crude residue, which was purified by column chromatography (silicagel, ethyl acetate in petroleum ether) to obtain a solid. The solid was crystallized using chloroform in petroleum ether to afford the title compound. Yield: 7.2 g (83%); ¹H NMR (CDCl₃, 300 MHz): δ 8.26 (d, 2H), 7.97 (s, 1H), 7.68 (d, 2H), 3.72 (s, 3H), 3.3 (t, 2H), 2.9 (t, 2H); MS: m/z 293 (M+1).

### Example 5

### Methyl 3-(5-(4-aminophenyl)thiazol-2-yl)propanoate

The compound of example 4 (4g) was dissolved in ethanol (40 mL), tetrahydrofuran (16 mL) and water (16 mL). Ammonium chloride (2.4 g) and iron (1.8 g) was added and refluxed at 80° C. for 3 h. The reaction mixture was cooled and filtered through Celite®. The reaction mixture was concentrated to yield a residue to which water was added followed by extraction with ethyl acetate. The organic layer was dried over anhydrous Na₂SO₄ and concentrated to obtain a crude residue, which was purified by column chromatography (silicagel, EtOAc in petroleum ether) to yield a solid. The solid was crystallized using EtOAc in petroleum ether to afford the title compound.

Yield: 3g (83%); ¹H NMR (DMSO-d₆, 300 MHz): δ 7.7 (s, 1H), 7.24 (d, 2H), 6.57 (d, 2H), 5.36 (bs, 2H), 3.59 (s, 3H), 3.16 (t, 2H), 2.78 (t, 2H); MS: m/z 263 (M+1).

### Example 6

### Methyl 3-(5-(4-(3-(3-(trifluoromethyl)phenyl)ureido)phenyl)thiazol-2-yl)propanoate

The compound of example 5 (150 mg) was dissolved in tetrahydrofuran (3 mL) to which was added 1-isocyanato-3-trifluoromethyl benzene (128 mg). The reaction mixture was stirred at room temperature for about 16 h. The reaction mixture was filtered to afford the title compound. Yield: 207 mg (80%); ¹H NMR (DMSO-d₆, 300 MHz): δ 9.06 (s, 1H), 8.94 (s, 1H), 8.0 (d, 1H), 7.93 (s, 1H), 7.55 (dd, 1H), 7.52 (d, 4H), 7.5 (m, 1H), 7.31 (dd, 1H), 3.59 (s, 3H), 3.21 (t, 2H), 2.81 (t, 2H); MS: m/z 450 (M+1).

### Example 7

### 3-(5-(4-(3-(3-(Trifluoromethyl)phenyl)ureido)phenyl)thiazol-2-yl)propanoic acid

The compound of example 6 (140 mg) was dissolved in tetrahydrofuran (2.8 mL) to which 1 M aqueous solution of lithium hydroxide monohydrate (0.62 mL) was added and stirred at room temperature for 6 h. The reaction mixture was acidified with dilute hydrochloric acid and extracted with ethyl acetate. The organic layer was separated out and dried over anhydrous Na₂SO₄. The organic layer was concentrated to obtain a solid, which was crystallized in ethyl acetate to afford the title compound. Yield: 100 mg (73%); ¹H NMR (DMSO-d₆, 300 MHz): δ 12.31 (bs, 1H), 9.09 (s, 1H), 8.97 (s, 1H), 8.02 (d, 1H), 7.95 (s, 1H), 7.57 (dd, 1H), 7.54 (d, 4H), 7.49 (m, 1H), 7.33 (dd, 1H), 3.19 (t, 2H), 2.74 (t, 2H); MS: m/z 436 (M+1).

### Example 8

### Methyl 3-(5-(4-(3-(2-chlorophenyl)ureido)phenyl)thiazol-2-yl)propanoate

The compound of example 8 was prepared analogous to the compound of example 6 by reaction of the compound of example 5 with 1-chloro-2-isocyanato benzene. Yield: 90%; ¹H NMR (DMSO-d₆, 300 MHz): δ 9.55 (s, 1H), 8.32 (s, 1H), 8.15 (dd, 1H), 7.93 (s, 1H), 7.52 (d, 4H), 7.43 (dd, 1H), 7.29 (m, 1H), 7.05 (m, 1H), 3.6 (s, 3H), 3.22 (t, 2H), 2.81 (t, 2H); MS: m/z 416 (M+1).

### Example 9

### 3-(5-(4-(3-(2-Chlorophenyl)ureido)phenyl)thiazol-2-yl)propanoic acid

The compound of example 9 was prepared analogous to the compound of example 7 by the hydrolysis of the compound of example 8. Yield: 91%; ¹H NMR (DMSO-d₆, 300 MHz): δ 12.26 (bs, 1H), 9.57 (s, 1H), 8.34 (s, 1H), 8.17 (dd, 1H), 7.95 (s, 1H), 7.54 (d, 4H), 7.45 (dd, 1H), 7.31 (m, 1H), 7.04 (m, 1H), 3.19 (t, 2H), 2.74 (t, 2H); MS: m/z 402 (M+1).

### Example 10

### Methyl 3-(5-(4-(3-cyclohexylureido)phenyl)thiazol-2-yl)propanoate

The compound of example 10 was prepared analogous to the compound of example 6 by reaction of the compound of example 5 with isocyanato cyclohexane.

Yield: 63%; ¹H NMR (DMSO-d₆, 300 MHz): δ 8.44 (s, 1H), 7.87 (s, 1H), 7.43 (d, 4H), 6.1 (d, 1H), 3.59 (s, 3H), 3.46 (m, 1H), 3.2 (t, 2H), 2.8 (t, 2H), 1.79 (m, 2H), 1.66-1.48 (m, 3H), 1.31-1.21 (m, 5H); MS: m/z 388 (M+1).

### Example 11

### 3-(5-(4-(3-Cyclohexylureido)phenyl)thiazol-2-yl)propanoic acid

The compound of example 11 was prepared analogous to the compound of example 7 by the hydrolysis of the compound of example 10. Yield: 51%; ¹H NMR (DMSO-d₆, 300 MHz): δ 12.26 (bs, 1H), 8.46 (s, 1H), 7.89 (s, 1H), 7.47-7.4 (d, 4H), 6.12 (d, 1H), 3.45 (m, 1H), 3.17 (t, 2H), 2.72 (t, 2H), 1.81 (m, 2H), 1.67-1.49 (m, 3H), 1.32-1.14 (m, 5H); MS: m/z 374 (M+1).

### Example 12

### Methyl 3-(5-(4-(3-(4-chloro-2-phenoxyphenyl)ureido)phenyl)thiazol-2-yl)propanoate

The compound of example 12 was prepared analogous to the compound of example 6 by reaction of the compound of example 5 with 4-chloro-1-isocyanato-2-phenoxy benzene. Yield: 96%; ¹H NMR (DMSO-d₆, 300 MHz): δ 9.51 (s, 1H), 8.7 (s, 1H), 8.4 (d, 1H), 7.95 (s, 1H), 7.56-7.46 (dd, 4H), 7.44-7.41 (dd, 2H), 7.2 (t, 1H), 7.1-7.08 (dd, 2H), 7.02-6.98 (dd, 1H), 6.85-6.82 (dd, 1H), 3.61 (s, 3H), 3.23 (t, 2H), 2.83 (t, 2H); MS: m/z 508 (M+1).

### Example 13

### 3-(5-(4-(3-(4-Chloro-2-phenoxyphenyl)ureido)phenyl)thiazol-2-yl)propanoic acid

The compound of example 13 was prepared analogous to the compound of example 7 by the hydrolysis of the compound of example 12. Yield: 77%; ¹H NMR (DMSO-d₆, 300 MHz): δ 12.3 (bs, 1H), 9.51 (s, 1H), 8.4 (s, 1H), 7.95 (d, 1H), 7.55 (d, 2H), 7.50 (d, 2H), 7.44 (dd, 2H), 7.2 (t, 1H), 7.1 (dd, 2H), 7.01-6.99 (dd, 1H), 6.85-6.83 (dd, 1H), 3.19 (t, 2H), 2.74 (t, 2H); MS: m/z 494 (M+1).

### Example 14

### Methyl 3-(5-(4-(4-tert-butylbenzamido)phenyl)thiazol-2-yl)propanoate

The compound of example 5 (150 mg) was dissolved in methylene chloride (3 mL), to which pyridine (0.138 mL) was added and the reaction mixture was stirred for 5 min. To this reaction mixture, 4-(t-butyl)benzoyl chloride (0.174 mL) was added and stirred for 3 hours. Water was added into the reaction mixture and the organic layer was separated and dried over anhydrous Na₂SO₄ to obtain a residue. The residue was purified by column chromatography (silicagel, EtOAc in chloroform) to yield a solid, which was crystallized using EtOAc in petroleum ether to afford the title compound. Yield: 168 (67%); ¹H NMR (DMSO-d₆, 300 MHz): δ 10.29 (s, 1H), 7.98 (s, 1H), 7.89 (d, 2H), 7.85 (d, 2H), 7.6 (d, 2H), 7.54 (d, 2H), 3.59 (s, 3H), 3.22 (t, 2H), 2.82 (t, 2H), 1.3 (s, 9H); MS: m/z 423 (M+1).

### Example 15

### 3-(5-(4-(4-Tert-butylbenzamido)phenyl)thiazol-2-yl)propanoic acid

The compound of example 14 (130 mg) was dissolved in tetrahydrofuran (2.6 mL) to which 1 M aqueous solution of Lithium hydroxide monohydrate (0.61 mL) was added and stirred at room temperature for 6 h. The reaction mixture was acidified with dilute hydrochloric acid and extracted with ethyl acetate. The organic layer was separated and dried over anhydrous Na₂SO₄. The organic layer was concentrated to obtain a solid, which was crystallized in ethyl acetate to afford the title compound. Yield: 80 mg (63%); ¹H NMR (DMSO-d₆, 300 MHz): δ 10.3 (s, 1H), 8.0 (s, 1H), 7.91 (d, 2H), 7.87 (d, 2H), 7.62 (d, 2H), 7.57 (d, 2H), 3.2 (t, 2H), 2.74 (t, 2H), 1.32 (s, 9H); MS: m/z 409 (M+1).

### Example 16

### Methyl 3-(5-(4-(4-pentylbenzamido)phenyl)thiazol-2-yl)propanoate

The compound of example 16 was prepared analogous to the compound of example 14 by reaction of the compound of example 5 with 4-pentyl-benzoyl chloride. Yield: 67%; ¹H NMR (DMSO-d₆, 300 MHz): δ 10.29 (s, 1H), 8.07 (s, 1H), 7.88 (d, 2H), 7.82 (d, 2H), 7.6 (d, 2H), 7.34 (d, 2H), 3.69 (s, 3H), 3.2 (t, 2H), 2.82 (t, 2H), 2.63 (t, 2H), 1.58 (m, 2H), 1.27 (m, 4H), 0.87 (t, 3H); MS: m/z 437 (M+1).

### Example 17

### 3-(5-(4-(4-Pentylbenzamido)phenyl)thiazol-2-yl)propanoic acid

The compound of example 17 was prepared analogous to the compound of example 15 by the hydrolysis of the compound of example 16. Yield: 62%; ¹H NMR (DMSO-d₆, 300 MHz): δ 12.3 (bs, 1H), 10.29 (s, 1H), 7.99 (s, 1H), 7.89 (d, 2H), 7.84 (d, 2H), 7.62 (d, 2H), 7.36 (d, 2H), 3.2 (t, 2H), 2.72 (t, 2H), 2.65 (t, 2H), 1.6 (m, 2H), 1.3 (m, 4H), 0.86 (t, 3H); MS: m/z 423 (M+1).

### Example 18

### Methyl 3-(5-(4-(3-ethoxy-5-(methoxymethyl)benzamido)phenyl)thiazol-2-yl)propanoate

The compound of example 18 was prepared analogous to the compound of example 14 by reaction of the compound of example 5 with 3-ethoxy-5-methoxymethyl-benzoyl chloride. Yield: 69%; ¹H NMR (DMSO-d₆, 300 MHz): δ 10.26 (s, 1H), 7.98 (s, 1H), 7.84 (d, 2H), 7.6 (d, 2H), 7.02 (d, 2H), 6.67 (m, 1H), 4.08 (q, 4H), 3.6 (s, 3H), 3.22 (t, 2H), 2.82 (t, 2H), 1.33 (t, 6H); MS: m/z 455 (M+1).

### Example 19

### 3-(5-(4-(3-Ethoxy-5-(methoxymethyl)benzamido)phenyl)thiazol-2-yl)propanoic acid

The compound of example 19 was prepared analogous to the compound of example 15 by the hydrolysis of the compound of example 18. Yield: 95%; ¹H NMR (DMSO-d₆, 300 MHz): δ 12.3 (bs, 1H), 10.26 (s, 1H), 8.0 (s, 1H), 7.86 (d, 2H), 7.62 (d, 2H), 7.09 (d, 2H), 6.69 (m, 1H), 4.08 (q, 4H), 3.2 (t, 2H), 2.74 (t, 2H), 1.35 (t, 6H); MS: m/z 441 (M+1).

### Example 20

### Methyl 3-(5-(4-(2-naphthamido)phenyl)thiazol-2-yl)propanoate

The compound of example 20 was prepared analogous to the compound of example 14 by reaction of the compound of example 5 with 2-naphthoyl chloride.

Yield: 88%; ¹H NMR (DMSO-d₆, 300 MHz): δ 10.57 (s, 1H), 8.59 (d, 1H), 8.1 (m, 2H), 8.04 (d, 2H), 8.01 (s, 1H), 7.9 (d, 2H), 7.66-7.59 (m, 4H), 3.6 (s, 3H), 3.23 (t, 2H), 2.82 (t, 2H); MS: m/z 417 (M+1).

### Example 21

### 3-(5-(4-(2-Naphthamido)phenyl)thiazol-2-yl)propanoic acid

The compound of example 21 was prepared analogous to the compound of example 15 by the hydrolysis of the compound of example 20. Yield: 64%; ¹H NMR (DMSO-d₆, 300 MHz): δ 12.31 (bs, 1H), 10.57 (s, 1H), 8.6 (d, 1H), 8.11 (m, 2H), 8.04 (d, 2H), 8.02 (s, 1H), 7.93 (d, 2H), 7.68-7.61 (m, 4H), 3.21 (t, 2H), 2.75 (t, 2H); MS: m/z 403 (M++1).

### Example 22

### Methyl 3-(5-(4-(4-butoxybenzamido)phenyl)thiazol-2-yl)propanoate

The compound of example 22 was prepared analogous to the compound of example 14 by reaction of the compound of example 5 with 4-butoxy-benzoyl chloride.

Yield: 94%; ¹H NMR (DMSO-d₆, 300 MHz): δ 10.20 (s, 1H), 7.97 (s, 1H), 7.92 (d, 2H), 7.82 (d, 2H), 7.59 (d, 2H), 7.05 (d, 2H), 4.04 (t, 2H), 3.6 (s, 3H), 3.22 (t, 2H), 2.82 (t, 2H), 1.71 (m, 2H), 1.44 (m, 2H), 0.93 (t, 3H); MS: m/z 439 (M+1).

### Example 23

### 3-(5-(4-(4-Butoxybenzamido)phenyl)thiazol-2-yl)propanoic acid

The compound of example 23 was prepared analogous to the compound of example 15 by the hydrolysis of the compound of example 22. Yield: 74%; ¹H NMR (DMSO-d₆, 300 MHz): δ 10.21 (s, 1H), 7.99 (s, 1H), 7.94 (d, 2H), 7.83 (d, 2H), 7.61 (d, 2H), 7.07 (d, 2H), 4.06 (t, 2H), 3.2 (t, 2H), 2.74 (t, 2H), 1.73 (m, 2H), 1.46 (m, 2H), 0.94 (t, 3H); MS: m/z 425 (M+1).

### Example 24

### Methyl 3-(5-(4-(2,4-dimethoxyphenylsulfonamido)phenyl)thiazol-2-yl)propanoate

The compound of example 5 (100 mg) was dissolved in methylene chloride (2 mL) to which pyridine (0.061 mL) was added and the reaction mixture was stirred for 5 min. To this reaction mixture, 2,4-dimethoxybenzene-1-sulfonyl chloride (0.135 g) was added and the reaction mixture was stirred for 16 h. Water was added into the reaction mixture and the reaction mixture was neutralized with dilute hydrochloric acid. The organic layer was washed with water and dried over anhydrous Na₂SO₄. The solvent was evaporated to obtain an oil, which was purified by column chromatography (silicagel, EtOAc in chloroform) to obtain a solid, which was crystallized using EtOAc in petroleum ether to afford the title compound. Yield: 153 (86%); ¹H NMR (DMSO-d₆, 300 MHz): δ 10.07 (s, 1H), 7.88 (s, 1H), 7.71 (d, 1H), 7.44 (d, 2H), 7.12 (d, 2H), 6.63 (d, 1H), 6.57 (dd, 1H), 3.86 (s, 3H), 3.78 (s, 3H), 3.59 (s, 3H), 3.22 (t, 2H), 2.79 (t, 2H); MS: m/z 463 (M+1).

### Example 25

### 3-(5-(4-(2,4-Dimethoxyphenylsulfonamido)phenyl)thiazol-2-yl)propanoic acid

The compound of example 24 (100 mg) was dissolved in tetrahydrofuran (2 mL) to which 1 M aqueous solution of lithium hydroxide monohydrate (0.43 mL) was added and stirred at room temperature for 6 h. The reaction mixture was acidified with dilute hydrochloric acid and extracted with ethyl acetate. The organic layer was separated, dried over anhydrous Na₂SO₄ and concentrated to obtain a solid which was crystallized in ethyl acetate to afford the title compound. Yield: 92 mg (94%); ¹H NMR (DMSO-d₆ 300 MHz): δ 12.27 (bs, 1H), 10.08 (s, 1H), 7.88 (s, 1H), 7.71 (d, 1H), 7.44 (d, 2H), 7.12 (d, 2H), 6.63 (d, 1H), 6.57 (dd, 1H), 3.86 (s, 3H), 3.78 (s, 3H), 3.18 (t, 2H), 2.7 (t, 2H); MS: m/z 449 (M+1).

### Example 26

### Methyl 2,2-dimethyl-4-(2-(4-nitrophenyl)-2-oxoethylamino)-4-oxobutanoate

Commercially available 4-methoxy-3,3-dimethyl-4-oxobutanoic acid (8g) was dissolved in tetrahydrofuran (160 mL) and to this solution, N-methyl morpholine (5.5 mL) was added. The reaction mixture was stirred for 10 min at room temperature and cooled to −20° C. Isobutyl chloroformate (6.48 mL) was added and the reaction mixture was stirred for 15-20 min at −20 to −30° C. The compound of example 2 (12.97 g) was neutralized with triethylamine (8.35 mL) in tetrahydrofuran (80 mL) and added to the reaction mixture with stirring at −20 to −30° C. for 5 min. The reaction mixture is gradually warmed to room temperature over a period of 1 h. The solvent is evaporated to obtain a crude residue, which was purified by column chromatograpy (silicagel, 25% ethyl acetate in chloroform) to afford the title compound. Yield: 8.8 g (54%); ¹H NMR (DMSO-d₆, 300 MHz): δ 8.38 (d, 2H), 8.15 (d, 2H), 6.74 (t, 1H), 4.8 (d, 2H), 3.77 (s, 3H), 2.63 (s, 2H), 1.33 (s, 6H); MS: m/z 323 (M+1).

### Example 27

### Methyl 2,2-dimethyl-3-(5-(4-nitrophenyl)thiazol-2-yl)propanoate

The compound of example 27 was prepared analogous to the compound of example 4 by reaction of the compound of example 26 with Lawesson's reagent.

Yield: 79%; ¹H NMR (CDCl₃, 300 MHz): δ 8.28 (d, 2H), 8.0 (s, 1H), 7.7 (d, 2H), 3.77 (s, 3H), 3.33 (s, 2H), 1.33 (s, 6H); MS: m/z 321 (M+1).

### Example 28

### Methyl 3-(5-(4-aminophenyl)thiazol-2-yl)-2,2-dimethylpropanoate

The compound of example 28 was prepared analogous to the compound of example 5 by reduction of the compound of example 27. Yield: 81%; ¹H NMR (DMSO-d₆, 300 MHz): δ 7.76 (s, 1H), 7.27 (d, 2H), 6.59 (d, 2H), 5.38 (bs, 2H), 3.64 (s, 3H), 3.16 (s, 2H), 1.23 (s, 6H); MS: m/z 291 (M+1).

### Example 29

### Methyl 3-(5-(4-(3-(2-chlorophenyl)ureido)phenyl)thiazol-2-yl)-2,2-dimethyl propanoate

The compound of example 29 was prepared analogous to the compound of example 6 by reaction of the compound of example 28 with 1-chloro-2-isocyanato benzene.

Yield: 83%; ¹H NMR (DMSO-d₆, 300 MHz): δ 9.57 (s, 1H), 8.34 (s, 1H), 8.17 (dd, 1H), 7.98 (s, 1H), 7.58-7.53 (dd, 4H), 7.48 (dd, 1H), 7.31 (m, 1H), 7.06 (m, 1H), 3.65 (s, 3H), 3.21 (s, 2H), 1.22 (s, 6H); MS: m/z 444 (M+1).

### Example 30

### 3-(5-(4-(3-(2-Chlorophenyl)ureido)phenyl)thiazol-2-yl)-2,2-diethylpropanoic acid

The compound of example 30 was prepared analogous to the compound of example 7 by hydrolysis of the compound of example 29. Yield: 91%; ¹H NMR (DMSO-d₆, 300 MHz): δ 12.45 (bs, 1H), 9.57 (s, 1H), 8.34 (s, 1H), 8.18 (dd, 1H), 7.98 (s, 1H), 7.57-7.54 (dd, 4H), 7.48 (dd, 1H), 7.31 (m, 1H), 7.04 (m, 1H), 3.18 (s, 2H), 1.19 (s, 6H); MS: m/z 430 (M+1).

### Example 31

### Methyl 2,2-dimethyl-3-(5-(4-(3-(4-(trifluoromethyl)phenyl)ureido)phenyl)thiazol-2-yl)propanoate

The compound of example 31 was prepared analogous to the compound of example 6 by reaction of the compound of example 30 with 1-isocyanato-4-trifluoromethyl benzene. Yield: 81%; ¹H NMR (DMSO-d₆, 300 MHz): δ 9.14 (s, 1H), 8.98 (s, 1H), 7.98 (s, 1H), 7.65 (dd, 4H), 7.55 (dd, 4H), 3.65 (s, 3H), 3.21 (s, 2H), 1.22 (s, 6H); MS: m/z 478 (M+1).

### Example 32

### 2,2-Dimethyl-3-(5-(4-(3-(4-(trifluoromethyl)phenyl)ureido)phenyl)thiazol-2-yl)propanoic acid

The compound of example 32 was prepared analogous to the compound of example 7 by hydrolysis of the compound of example 31. Yield: 94%; ¹H NMR (DMSO-d₆, 300 MHz): δ 12.46 (bs, 1H), 9.21 (s, 1H), 9.04 (s, 1H), 7.97 (s, 1H), 7.66 (dd, 4H), 7.54 (dd, 4H), 3.18 (s, 2H), 1.19 (s, 6H); MS: m/z 464 (M+1).

### Example 33

### Methyl 3-(5-(4-(3-(4-fluorophenyl)ureido)phenyl)thiazol-2-yl)-2,2-dimethyl propanoate

The compound of example 33 was prepared analogous to the compound of example 6 by reaction of the compound of example 28 with 1-fluoro-4-isocyanato benzene.

Yield: 75%; ¹H NMR (DMSO-d₆, 300 MHz): δ 8.83 (s, 1H), 8.73 (s, 1H), 7.96 (s, 1H), 7.52 (dd, 4H), 7.46 (d, 2H), 7.12 (d, 2H), 3.65 (s, 3H), 3.21 (s, 2H), 1.21 (s, 6H); MS: m/z 428 (M+1).

### Example 34

### 3-(5-(4-(3-(4-Fluorophenyl)ureido)phenyl)thiazol-2-yl)-2,2-dimethylpropanoic acid

The compound of example 34 was prepared analogous to the compound of example 7 by hydrolysis of the compound of example 33. Yield: 70%; ¹H NMR (DMSO-d₆, 300 MHz): δ 12.41 (bs, 1H), 8.95 (s, 1H), 8.85 (s, 1H), 7.96 (s, 1H), 7.52 (dd, 4H), 7.46 (d, 2H), 7.12 (d, 2H), 3.17 (s, 2H), 1.19 (s, 6H); MS: m/z 414 (M+1).

### Example 35

### Methyl 3-(5-(4-(3-(4-methoxyphenyl)ureido)phenyl)thiazol-2-yl)-2,2-dimethyl propanoate

The compound of example 35 was prepared analogous to the compound of example 6 by reaction of the compound of example 28 with 1-isocyanato-4-methoxybenzene.

Yield: 79%; ¹H NMR (DMSO-d₆, 300 MHz): δ 8.75 (s, 1H), 8.5 (s, 1H), 7.96 (s, 1H), 7.51 (dd, 4H), 7.37 (d, 2H), 6.89 (d, 2H), 3.72 (s, 3H), 3.65 (s, 3H), 3.23 (s, 2H), 1.22 (s, 6H); MS: m/z 440 (M+1).

### Example 36

### 3-(5-(4-(3-(4-Methoxyphenyl)ureido)phenyl)thiazol-2-yl)-2,2-dimethyl propanoic acid

The compound of example 36 was prepared analogous to the compound of example 7 by hydrolysis of the compound of example 35. Yield: 60%; ¹H NMR (DMSO-d₆, 300 MHz): δ 12.46 (bs, 1H), 9.17 (s, 1H), 9.15 (s, 1H), 7.93 (s, 1H), 7.5 (dd, 4H), 7.39 (d, 2H), 6.88 (d, 2H), 3.71 (s, 3H), 3.17 (s, 2H), 1.18 (s, 6H); MS: m/z 426 (M+1).

### Example 37

### Methyl 3-(5-(4-(3-cyclohexylureido)phenyl)thiazol-2-yl)-2,2-dimethyl propanoate

The compound of example 37 was prepared analogous to the compound of example 6 by reaction of the compound of example 28 with isocyanato cyclohexane. Yield: 78%;

¹H NMR (DMSO-d₆, 300 MHz): δ 8.47 (s, 1H), 7.92 (s, 1H), 7.45 (dd, 4H), 6.12 (d, 1H), 3.64 (s, 3H), 3.46 (m, 1H), 3.2 (s, 2H), 1.81 (m, 2H), 1.63 (m, 2H), 1.52 (m, 1H), 1.33 (m, 2H), 1.21 (s, 6H), 1.14 (m, 3H); MS: m/z 430 (M+1).

### Example 38

### 3-(5-(4-(3-Cyclohexylureido)phenyl)thiazol-2-yl)-2,2-dimethylpropanoic acid

The compound of example 38 was prepared analogous to the compound of example 7 by hydrolysis of the compound of example 37. Yield: 94%; ¹H NMR (DMSO-d₆, 300 MHz): δ 8.57 (s, 1H), 7.92 (s, 1H), 7.44 (dd, 4H), 6.18 (d, 1H), 3.47 (m, 1H), 3.16 (s, 2H), 1.81 (m, 2H), 1.64 (m, 2H), 1.53 (m, 1H), 1.32 (m, 2H), 1.18 (m, 9H); MS: m/z 402 (M+1).

### Example 39

### Methyl 3-(5-(4-(3-(4-chloro-2-phenoxyphenyl)ureido)phenyl)thiazol-2-yl)-2,2-dimethylpropanoate

The compound of example 39 was prepared analogous to the compound of example 6 by reaction of the compound of example 28 with 4-chloro-1-isocyanato-2-phenoxy benzene. Yield: 90%; ¹H NMR (DMSO-d₆, 300 MHz): δ 9.51 (s, 1H), 8.69 (s, 1H), 8.39 (d, 1H), 7.98 (s, 1H), 7.57-7.51 (dd, 4H), 7.44 (dd, 2H), 7.2 (t, 1H), 7.1 (dd, 2H), 7.02-6.98 (dd, 1H), 6.85-6.82 (dd, 1H), 3.65 (s, 3H), 3.21 (s, 2H), 1.21 (s, 6H); MS: m/z 536 (M+1).

### Example 40

### 3-(5-(4-(3-(4-Chloro-2-phenoxyphenyl)ureido)phenyl)thiazol-2-yl)-2,2-dimethylpropanoic acid

The compound of example 40 was prepared analogous to the compound of example 7 by hydrolysis of the compound of example 39. Yield: 87%; ¹H NMR (DMSO-d₆, 300 MHz): δ 12.46 (bs, 1H), 9.55 (s, 1H), 8.77 (s, 1H), 8.39 (d, 1H), 7.97 (s, 1H), 7.56-7.51 (dd, 4H), 7.44 (d, 2H), 7.19 (t, 1H), 7.1 (dd, 2H), 6.99 (dd, 1H), 6.85 (dd, 1H), 3.17 (s, 2H), 1.19 (s, 6H); MS: m/z 522 (M+1).

### Example 41

### Methyl 3-(5-(4-(4-tert-butyl benzamido)phenyl)thiazol-2-yl)-2,2-dimethyl propanoate

The compound of example 41 was prepared analogous to the compound of example 14 by reaction of the compound of example 28 with 4-(t-butyl)benzoyl chloride. Yield: 70%; ¹H NMR (DMSO-d₆, 300 MHz): δ 10.32 (s, 1H), 8.03 (s, 1H), 7.91-7.84 (dd, 4H), 7.63-7.54 (dd, 4H), 3.65 (s, 3H), 3.22 (s, 2H), 1.32 (s, 9H), 1.22 (s, 6H); MS: m/z 451 (M+1).

### Example 42

### 3-(5-(4-(4-tert-Butylbenzamido)phenyl)thiazol-2-yl)-2,2-dimethylpropanoic acid

The compound of example 42 was prepared analogous to the compound of example 15 by hydrolysis of the compound of example 41. Yield: 77%; ¹H NMR (DMSO-d₆, 300 MHz): δ 12.31 (bs, 1H), 10.31 (s, 1H), 8.03 (s, 1H), 7.91-7.84 (dd, 4H), 7.62-7.54 (dd, 4H), 3.22 (s, 2H), 1.32 (s, 9H), 1.19 (s, 6H); MS: m/z 437 (M+1).

### Example 43

### Methyl 3-(5-(4-biphenyl-4-ylcarboxamidophenyl)thiazol-2-yl)-2,2-dimethyl propanoate

The compound of example 43 was prepared analogous to the compound of example 14 by reaction of the compound of example 28 with 4-phenyl-benzoyl chloride. Yield: 81%; ¹H NMR (DMSO-d₆, 300 MHz): δ 10.44 (s, 1H), 8.09 (d, 2H), 8.04 (s, 1H), 7.93-7.84 (dd, 4H), 7.78 (dd, 2H), 7.65 (dd, 2H), 7.52 (dd, 2H), 7.43 (dd, 1H), 3.66 (s, 3H), 3.23 (s, 2H), 1.23 (s, 6H); MS: m/z 471 (M+1).

### Example 44

### 3-(5-(4-Biphenyl-4-ylcarboxamidophenyl)thiazol-2-yl)-2,2-dimethylpropanoic acid

The compound of example 44 was prepared analogous to the compound of example 15 by hydrolysis of the compound of example 43. Yield: 62%; ¹H NMR (DMSO-d₆, 300 MHz): δ 12.46 (bs, 1H), 10.43 (s, 1H), 8.09 (d, 2H), 8.03 (s, 1H), 7.91-7.84 (d, 2H), 7.78 (dd, 2H), 7.64 (d, 2H), 7.52 (dd, 2H), 7.43 (dd, 1H), 3.19 (s, 2H), 1.2 (s, 6H); MS: m/z 457 (M+1).

### Example 45

### Methyl 5-(2-(4-nitrophenyl)-2-oxoethylamino)-5-oxopentanoate

The compound of example 45 is prepared analogous to the compound of example 3 by reaction of the compound of example 2 with methyl 5-chloro-5-oxopentanoate. Yield: 34%; ¹H NMR (DMSO-d₆, 300 MHz): δ 8.36 (t, 1H), 8.33 (d, 2H), 8.2 (d, 2H), 4.63 (d, 2H), 3.58 (s, 3H), 2.29 (t, 2H), 2.21 (t, 2H), 1.74 (m, 2H); MS: m/z 309 (M+1).

### Example 46

### Methyl 4-(5-(4-nitrophenyl)thiazol-2-yl)butanoate

The compound of example 46 is prepared analogous to the compound of example 4 by reaction of the compound of example 45 with Lawesson's reagent.

Yield: 82%; ¹H NMR (CDCl₃, 300 MHz): δ 8.29 (d, 2H), 8.0 (s, 1H), 7.71 (d, 2H), 3.71 (s, 3H), 3.13 (t, 2H), 2.49 (t, 2H), 2.20 (m, 2H); MS: m/z 307 (M+1).

### Example 47

### Methyl 4-(5-(4-aminophenyl)thiazol-2-yl)butanoate

The compound of example 47 was prepared analogous to the compound of example 5 by reduction of the compound of example 46. Yield: 89%; ¹H NMR (DMSO-d₆, 300 MHz): δ 7.74 (s, 1H), 7.27 (d, 2H), 6.59 (d, 2H), 5.38 (bs, 2H), 3.59 (s, 3H), 2.94 (t, 2H), 2.42 (t, 2H); 1.96 (m, 2H); MS: m/z 277 (M+1).

### Example 48

### Methyl 4-(5-(4-(3-(3-(trifluoromethyl)phenyl)ureido)phenyl)thiazol-2-yl)butanoate

The compound of example 48 was prepared analogous to the compound of example 6 by reaction of the compound of example 47 with 1-isocyanato-3-trifluoromethyl benzene. Yield: 73%; ¹H NMR (DMSO-d₆, 300 MHz): δ 9.09 (s, 1H), 8.97 (s, 1H), 8.02 (d, 1H), 7.91 (s, 1H), 7.6 (dd, 1H), 7.54 (d, 4H), 7.49 (m, 1H), 7.33 (dd, 1H), 3.6 (s, 3H), 2.99 (t, 2H), 2.44 (t, 2H), 1.98 (m, 2H); MS: m/z 464 (M+1).

### Example 49

### 4-(5-(4-(3-(3-(Trifluoromethyl)phenyl)ureido)phenyl)thiazol-2-yl)butanoic acid

The compound of example 49 was prepared analogous to the compound of example 7 by hydrolysis of the compound of example 48. Yield: 71%; ¹H NMR (DMSO-d₆, 300 MHz): δ 12.12 (bs, 1H), 9.11 (s, 1H), 8.99 (s, 1H), 8.02 (d, 1H), 7.97 (s, 1H), 7.6 (dd, 1H), 7.55 (d, 4H), 7.49 (m, 1H), 7.33 (dd, 1H), 2.99 (t, 2H), 2.35 (t, 2H), 1.95 (m, 2H); MS: m/z 450 (M+1).

### Example 50

### Methyl 4-(5-(4-(3-(2-chlorophenyl)ureido)phenyl)thiazol-2-yl)butanoate

The compound of example 50 was prepared analogous to the compound of example 6 by reaction of the compound of example 47 with 1-chloro-2-isocyanato benzene. Yield: 88%; ¹H NMR (DMSO-d₆, 300 MHz): δ 9.57 (s, 1H), 8.35 (s, 1H), 8.19 (dd, 1H), 7.97 (s, 1H), 7.55 (d, 4H), 7.45 (dd, 1H), 7.31 (m, 1H), 7.04 (m, 1H), 3.6 (s, 3H), 3.0 (t, 2H), 2.44 (t, 2H), 1.98 (m, 2H); MS: m/z 430 (M+1).

### Example 51

### 4-(5-(4-(3-(2-Chlorophenyl)ureido)phenyl)thiazol-2-yl)butanoic acid

The compound of example 51 was prepared analogous to the compound of example 7 by hydrolysis of the compound of example 50. Yield: 84%; ¹H NMR (DMSO-d₆, 300 MHz): δ 12.12 (bs, 1H), 9.64 (s, 1H), 8.39 (s, 1H), 8.17 (dd, 1H), 7.96 (s, 1H), 7.55 (d, 4H), 7.45 (dd, 1H), 7.31 (m, 1H), 7.04 (m, 1H), 2.99 (t, 2H), 2.34 (t, 2H), 1.95 (m, 2H); MS: m/z 416 (M+1).

### Example 52

### Methyl 4-(5-(4-(3-(3,4-dimethylphenyl)ureido)phenyl)thiazol-2-yl)butanoate

The compound of example 52 was prepared analogous to the compound of example 6 by reaction of the compound of example 47 with 4-isocyanato-1,2-dimethyl benzene.

Yield: 82%; ¹H NMR (DMSO-d₆, 300 MHz): δ 8.78 (s, 1H), 8.52 (s, 1H), 7.95 (s, 1H), 7.51 (d, 4H), 7.23 (d, 1H), 7.15 (dd, 1H), 7.04 (d, 1H), 3.6 (s, 3H), 2.99 (t, 2H), 2.44 (t, 2H), 2.19 (s, 3H), 2.15 (s, 3H), 1.98 (m, 2H); MS: m/z 424 (M+1).

### Example 53

### 4-(5-(4-(3-(3,4-Dimethylphenyl)ureido)phenyl)thiazol-2-yl)butanoic acid

The compound of example 53 was prepared analogous to the compound of example 7 by hydrolysis of the compound of example 52. Yield: 91%; ¹H NMR (DMSO-d₆, 300 MHz): δ 12.14 (bs, 1H), 8.82 (s, 1H), 8.55 (s, 1H), 7.95 (s, 1H), 7.52 (d, 4H), 7.23 (d, 1H), 7.16 (dd, 1H), 7.04 (d, 1H), 2.99 (t, 2H), 2.37 (t, 2H), 2.19 (s, 3H), 2.15 (s, 3H), 1.95 (m, 2H); MS: m/z 410 (M+1).

### Example 54

### Methyl 4-(5-(4-(3-(4-chloro-2-phenoxyphenyl)ureido)phenyl)thiazol-2-yl)butanoate

The compound of example 54 was prepared analogous to the compound of example 6 by reaction of the compound of example 47 with 4-chloro-1-isocyanato-2-phenoxy benzene. Yield: 96%; ¹H NMR (DMSO-d₆, 300 MHz): δ 9.51 (s, 1H), 8.7 (s, 1H), 8.4 (d, 1H), 7.96 (s, 1H), 7.54-7.51 (dd, 4H), 7.44-7.41 (dd, 2H), 7.22 (t, 1H), 7.1-7.08 (dd, 2H), 7.02-6.98 (dd, 1H), 6.85-6.82 (dd, 1H), 3.6 (s, 3H), 2.99 (t, 2H), 2.44 (t, 2H), 1.98 (m, 2H); MS: m/z 522 (M+1).

### Example 55

### 4-(5-(4-(3-(4-Chloro-2-phenoxyphenyl)ureido)phenyl)thiazol-2-yl)butanoic acid

The compound of example 55 was prepared analogous to the compound of example 7 by hydrolysis of the compound of example 54. Yield: 89%; ¹H NMR (DMSO-d₆, 300 MHz): δ 12.15 (bs, 1H), 9.51 (s, 1H), 8.7 (s, 1H), 8.39 (d, 1H), 7.96 (s, 1H), 7.55 (d, 2H), 7.49 (d, 2H), 7.44 (dd, 2H), 7.21 (t, 1H), 7.1 (dd, 2H), 7.01-6.99 (dd, 1H), 6.85-6.83 (dd, 1H), 2.99 (t, 2H), 2.34 (t, 2H), 1.95 (m, 2H); MS: m/z 508 (M+1).

### Example 56

### Methyl 4-(5-(4-(4-tert-butylbenzamido)phenyl)thiazol-2-yl)butanoate

The compound of example 56 was prepared analogous to the compound of example 14 by reaction of the compound of example 47 with 4-(t-butyl)benzoyl chloride. Yield:

85%; ¹H NMR (DMSO-d₆, 300 MHz): δ 10.33 (s, 1H), 8.03 (s, 1H), 7.92-7.85 (dd, 4H), 7.63-7.54 (dd, 4H), 3.6 (s, 3H), 3.01 (t, 2H), 2.45 (t, 2H), 1.99 (m, 2H), 1.32 (s, 9H); MS: m/z 437 (M+1).

### Example 57

### 4-(5-(4-(4-Tert-butylbenzamido)phenyl)thiazol-2-yl)butanoic acid

The compound of example 57 was prepared analogous to the compound of example 15 by hydrolysis of the compound of example 56. Yield: 62%; ¹H NMR (DMSO-d₆, 300 MHz): δ 12.15 (bs, 1H), 10.3 (s, 1H), 8.01 (s, 1H), 7.91-7.84 (dd, 4H), 7.63-7.54 (dd, 4H), 3.0 (t, 2H), 2.35 (t, 2H), 1.96 (m, 2H), 1.32 (s, 9H); MS: m/z 423 (M+1).

### Example 58

### Methyl 4-(5-(4-(4-pentylbenzamido)phenyl)thiazol-2-yl)butanoate

The compound of example 58 was prepared analogous to the compound of example 14 by reaction of the compound of example 47 with 4-pentylbenzoyl chloride. Yield: 90%; ¹H NMR (DMSO-d₆, 300 MHz): δ 10.31 (s, 1H), 8.01 (s, 1H), 7.9-7.84 (dd, 4H), 7.63 (d, 2H), 7.37 (d, 2H), 3.6 (s, 3H), 3.03 (t, 2H), 2.63 (t, 2H), 2.45 (t, 2H), 2.01 (m, 2H), 1.61 (m, 2H), 1.29 (m, 4H), 0.86 (t, 3H); MS: m/z 451 (M+1).

### Example 59

### 4-(5-(4-(4-Pentylbenzamido)phenyl)thiazol-2-yl)butanoic acid

The compound of example 59 was prepared analogous to the compound of example 15 by hydrolysis of the compound of example 58. Yield: 81%; ¹H NMR (DMSO-d₆, 300 MHz): δ 12.14 (bs, 1H), 10.3 (s, 1H), 8.01 (s, 1H), 7.9-7.84 (dd, 4H), 7.63 (d, 2H), 7.37 (d, 2H), 3.0 (t, 2H), 2.65 (t, 2H), 2.35 (t, 2H), 1.96 (m, 2H), 1.60 (m, 2H), 1.29 (m, 4H), 0.86 (t, 3H); MS: m/z 437 (M+1).

### Example 60

### Methyl 4-(5-(4-biphenyl-4-ylcarboxamidophenyl)thiazol-2-yl)butanoate

The compound of example 60 was prepared analogous to the compound of example 14 by reaction of the compound of example 47 with 4-phenylbenzoyl chloride. Yield: 35%; ¹H NMR (DMSO-d₆, 300 MHz): δ 10.44 (s, 1H), 8.09 (d, 2H), 8.03 (s, 1H), 7.9-7.84 (dd, 4H), 7.78 (dd, 2H), 7.65 (dd, 2H), 7.52 (dd, 2H), 7.43 (dd, 1H), 3.61 (s, 3H), 3.01 (t, 2H), 2.45 (t, 2H), 1.99 (m, 2H); MS: m/z 457 (M+1).

### Example 61

### 4-(5-(4-Biphenyl-4-ylcarboxamidophenyl)thiazol-2-yl)butanoic acid

The compound of example 61 was prepared analogous to the compound of example 15 by hydrolysis of the compound of example 60. Yield: 75%; ¹H NMR (DMSO-d₆, 300 MHz): δ 10.44 (s, 1H), 8.12 (s, 1H), 8.09 (d, 2H), 7.93 (d, 2H), 7.85 (d, 2H), 7.76 (dd, 2H), 7.66 (d, 2H), 7.5 (dd, 2H), 7.43 (dd, 1H), 3.06 (t, 2H), 2.36 (t, 2H), 1.98 (m, 2H); MS: m/z 443 (M+1).

### Example 62

### Methyl 4-(5-(4-(2,4-dimethoxyphenylsulfonamido)phenyl)thiazol-2-yl)butanoate

The compound of example 62 was prepared analogous to the compound of example 24 by reaction of the compound of example 47 with 2,4-dimethoxybenzene-1-sulfonyl chloride. Yield: 85%; ¹H NMR (DMSO-d₆, 300 MHz): δ 10.08 (s, 1H), 7.89 (s, 1H), 7.71 (d, 1H), 7.45 (d, 2H), 7.12 (d, 2H), 6.63 (d, 1H), 6.57 (dd, 1H), 3.86 (s, 3H), 3.78 (s, 3H), 3.58 (s, 3H), 2.96 (t, 2H), 2.41 (t, 2H), 1.94 (m, 2H); MS: m/z 477 (M+1).

### Example 63

### 4-(5-(4-(2,4-Dimethoxyphenylsulfonamido)phenyl)thiazol-2-yl)butanoic acid

The compound of example 63 was prepared analogous to the compound of example 25 by hydrolysis of the compound of example 62. Yield: 69%; ¹H NMR (DMSO-D6, 300 MHz): δ 12.07 (bs, 1H), 10.08 (s, 1H), 7.9 (s, 1H), 7.71 (d, 1H), 7.45 (d, 2H), 7.12 (d, 2H), 6.63 (d, 1H), 6.57 (dd, 1H), 3.86 (s, 3H), 2.95 (t, 2H), 2.31 (t, 2H), 1.94 (m, 2H); MS: m/z 463 (M+1).

### Example 64

### 5-Methoxy-3,3-dimethyl-5-oxopentanoic acid

Sodium metal (1.29 g) was dissolved in dry methanol (80 mL). To this solution, 4,4-dimethyldihydro-2H-pyran-2,6(3H)-dione (4g) was added and refluxed for 3 h. The reaction mixture was cooled and poured into ice-water. Diethyl ether was added and 2N HCl was added to adjust the pH to 2 with 2N HCl. The layers were separated and the aqueous layer was extracted with diethyl ether. The organic layer was dried over anhydrous sodium sulphate, filtered and concentrated to afford the title compound.

Yield: 4.7 g (95%); ¹H NMR (DMSO-d₆, 300 MHz): δ 12.03 (bs, 1H), 3.57 (s, 3H), 2.11 (s, 2H), 2.25 (s, 2H), 1.04 (s, 6H); MS: m/z 173 (M−1).

### Example 65

### Methyl 3,3-dimethyl-5-(2-(4-nitrophenyl)-2-oxoethylamino)-5-oxopentanoate

The compound of example 65 was prepared analogous to the compound of example 26 by reaction of the compound of example 2 with the compound of example 64. Yield 6.5 g (73%); ¹H NMR (DMSO-d₆, 300 MHz): δ 8.33 (d, 2H), 8.27 (t, 1H), 8.18 (d, 2H), 4.63 (d, 2H), 3.57 (s, 3H), 2.37 (s, 2H), 2.22 (s, 2H), 1.03 (s, 6H); MS: m/z 337 (M+1).

### Example 66

### Methyl 3,3-dimethyl-4-(5-(4-nitrophenyl)thiazol-2-yl)butanoate

The compound of example 66 is prepared analogous to the compound of example 4 by reaction of the compound of example 65 with Lawesson's reagent. Yield: 57%; ¹H NMR (CDCl₃, 300 MHz): δ 8.29 (d, 2H), 8.0 (s, 1H), 7.72 (d, 2H), 3.72 (s, 3H), 3.16 (s, 2H), 2.4 (s, 2H), 1.1 (s, 6H); MS: m/z 335 (M+1).

### Example 67

### Methyl 4-(5-(4-aminophenyl)thiazol-2-yl-3,3-dimethylbutanoate

The compound of example 67 is prepared analogous to the compound of example 5 by reduction of the compound of example 66. Yield: 91%; ¹H NMR (DMSO-d₆, 300 MHz): δ 7.8 (s, 1H), 7.28 (d, 2H), 6.59 (d, 2H), 5.38 (bs, 2H), 3.59 (s, 3H), 2.97 (s, 2H), 2.35 (s, 2H); 1.03 (s, 6H); MS: m/z 305 (M+1).

### Example 68

### Methyl 3,3-dimethyl-4-(5-(4-(3-(3-(trifluoromethyl)phenyl)ureido)phenyl)thiazol-2-yl)butanoate

The compound of example 68 was prepared analogous to the compound of example 6 by reaction of the compound of example 67 with 1-isocyanato-3-trifluoromethyl benzene. Yield: 193 mg (79%); ¹H NMR (DMSO-d₆, 300 MHz): δ 9.09 (s, 1H), 8.97 (s, 1H), 8.02 (d, 2H), 7.58 (s, 1H), 7.54 (d, 4H), 7.52 (dd, 1H), 7.33 (m, 1H), 3.6 (s, 3H), 3.02 (s, 2H), 2.37 (s, 2H), 1.05 (s, 6H); MS: m/z 490 (M+1).

### Example 69

### 3,3-Dimethyl-4-(5-(4-(3-(3-(trifluoromethyl)phenyl)ureido)phenyl)thiazol-2-yl)butanoic acid

The compound of example 69 was prepared analogous to the compound of example 7 by hydrolysis of the compound of example 68. Yield: 93%; ¹H NMR (DMSO-d₆, 300 MHz): δ 12.11 (bs, 1H), 9.12 (s, 1H), 9.01 (s, 1H), 8.02 (d, 2H), 7.6-7.49 (m, 6H), 7.33 (dd, 1H), 3.04 (s, 2H), 2.26 (s, 2H), 1.06 (s, 6H); MS: m/z 478 (M+1).

### Example 70

### Methyl 4-(5-(4-(3-(2-chlorophenyl)ureido)phenyl)thiazol-2-yl)-3,3-dimethyl butanoate

The compound of example 70 was prepared analogous to the compound of example 6 by reaction of the compound of example 67 with 1-chloro-2-isocyanato benzene. Yield: 84%; ¹H NMR (DMSO-d₆, 300 MHz): δ 9.57 (s, 1H), 8.35 (s, 1H), 8.18 (dd, 1H), 8.02 (s, 1H), 7.59-7.51 (d, 4H), 7.45 (dd, 1H), 7.31 (m, 1H), 7.04 (m, 1H), 3.6 (s, 3H), 3.02 (s, 2H), 2.37 (s, 2H), 1.05 (s, 6H); MS: m/z 458 (M+1).

### Example 71

### 4-(5-(4-(3-(2-Chlorophenyl)ureido)phenyl)thiazol-2-yl)-3,3-dimethyl butanoic acid

The compound of example 71 was prepared analogous to the compound of example 7 by hydrolysis of the compound of example 70. Yield: 55%; ¹H NMR (DMSO-d₆, 300 MHz): δ 12.10 (bs, 1H), 9.57 (s, 1H), 8.34 (s, 1H), 8.17 (dd, 1H), 8.02 (s, 1H), 7.59-7.51 (d, 4H), 7.48 (dd, 1H), 7.31 (m, 1H), 7.04 (m, 1H), 3.04 (s, 2H), 2.26 (s, 2H), 1.06 (s, 6H); MS: m/z 444 (M+1).

### Example 72

### Methyl 4-(5-(4-(3-(4-chloro-2-phenoxyphenyl)ureido)phenyl)thiazol-2-yl)-3,3-dimethylbutanoate

The compound of example 72 was prepared analogous to the compound of example 6 by reaction of the compound of example 67 with 4-chloro-1-isocyanato-2-phenoxy benzene. Yield: 83%; ¹H NMR (DMSO-d₆, 300 MHz): 9.51 (s, 1H), 8.7 (s, 1H), 8.4 (d, 1H), 8.02 (s, 1H), 7.58-7.51 (dd, 4H), 7.48-7.41 (dd, 2H), 7.2 (t, 1H), 7.1 (dd, 2H), 6.99 (dd, 1H), 6.85 (dd, 1H), 3.6 (s, 3H), 3.02 (s, 2H), 2.37 (s, 2H), 1.05 (s, 6H); MS: m/z 550 (M+1).

### Example 73

### 4-(5-(4-(3-(4-Chloro-2-phenoxyphenyl)ureido)phenyl)thiazol-2-yl)-3,3-dimethyl butanoic acid

The compound of example 73 was prepared analogous to the compound of example 7 by hydrolysis of the compound of example 72. Yield: 65%; ¹H NMR (DMSO-d₆, 300 MHz): 12.1 (bs, 1H), 9.51 (s, 1H), 8.69 (s, 1H), 8.39 (d, 1H), 8.02 (s, 1H), 7.58-7.41 (ddd, 6H), 7.19 (t, 1H), 7.1 (dd, 2H), 6.99 (dd, 1H), 6.85 (dd, 1H), 3.04 (s, 2H), 2.26 (s, 2H), 1.06 (s, 6H); MS: m/z 536 (M+1).

### Example 74

### Methyl 4-(5-(4-(4-tert-butylbenzamido)phenyl)thiazol-2-yl)-3,3-dimethyl butanoate

The compound of example 74 was prepared analogous to the compound of example 14 by reaction of the compound of example 67 with 4-(t-butyl)benzoyl chloride. Yield: 85%; ¹H NMR (DMSO-d₆, 300 MHz): δ 10.32 (s, 1H), 8.07 (s, 1H), 7.91-7.85 (dd, 4H), 7.64-7.54 (dd, 4H), 3.6 (s, 3H), 3.04 (s, 2H), 2.37 (s, 2H), 1.32 (s, 9H), 1.06 (s, 6H); MS: m/z 465 (M+1).

### Example 75

### 4-(5-(4-(4-tert-Butylbenzamido)phenyl)thiazol-2-yl)-3,3-dimethylbutanoic acid

The compound of example 75 was prepared analogous to the compound of example 15 by hydrolysis of the compound of example 74. Yield: 71%; ¹H NMR (DMSO-d₆, 300 MHz): δ 12.11 (bs, 1H), 10.3 (s, 1H), 8.06 (s, 1H), 7.91-7.84 (dd, 4H), 7.64-7.54 (dd, 4H), 3.05 (s, 2H), 2.27 (s, 2H), 1.32 (s, 9H), 1.06 (s, 6H); MS: m/z 451 (M+1).

### Example 76

### Methyl 4-(5-(4-bi phenyl-4-ylcarboxamidophenyl)thiazol-2-yl)-3,3-dimethyl butanoate

The compound of example 76 was prepared analogous to the compound of example 14 by reaction of the compound of example 67 with 4-phenylbenzoyl chloride. Yield: 58%; ¹H NMR (DMSO-d₆, 300 MHz): δ 10.43 (s, 1H), 8.09 (d, 2H), 8.07 (s, 1H), 7.9-7.85 (dd, 4H), 7.78 (dd, 2H), 7.66 (dd, 2H), 7.52 (dd, 2H), 7.43 (dd, 1H), 3.61 (s, 3H), 3.04 (s, 2H), 2.38 (s, 2H), 1.06 (s, 6H); MS: m/z 485 (M+1).

### Example 77

### 4-(5-(4-Biphenyl-4-ylcarboxamidophenyl)thiazol-2-yl)-3,3-dimethylbutanoic acid

The compound of example 77 was prepared analogous to the compound of example 15 by hydrolysis of the compound of example 76. Yield: 68%; ¹H NMR (DMSO-d₆, 300 MHz): δ 12.11 (bs, 1H), 10.43 (s, 1H), 8.09 (d, 2H), 8.06 (s, 1H), 7.91-7.84 (dd, 4H), 7.78 (dd, 2H), 7.66 (dd, 2H), 7.52 (dd, 2H), 7.43 (dd, 1H), 3.06 (s, 2H), 2.27 (s, 2H), 1.07 (s, 6H); MS: m/z 471 (M+1).

### Example 78

### Methyl 3,3-dimethyl-4-(5-(4-(4-pentyl benzamido)phenyl)thiazol-2-yl)butanoate

The compound of example 78 was prepared analogous to the compound of example 14 by reaction of the compound of example 67 with 4-pentylbenzoyl chloride. Yield: 89%; ¹H NMR (DMSO-d₆, 300 MHz): δ 10.31 (s, 1H), 8.09 (s, 1H), 7.91-7.86 (dd, 4H), 7.64 (d, 2H), 7.36 (d, 2H), 3.6 (s, 3H), 3.04 (s, 2H), 2.65 (t, 2H), 2.37 (s, 2H), 1.6 (m, 2H), 1.29 (m, 4H), 1.06 (s, 6H), 0.926 (t, 3H); MS: m/z 479 (M+1).

### Example 79

3,3-Dimethyl-4-(5-(4-(4-pentylbenzamido)phenyl)thiazol-2-yl)butanoic acid

The compound of example 79 was prepared analogous to the compound of example 15 by hydrolysis of the compound of example 78. Yield: 64%; ¹H NMR (DMSO-d₆, 300 MHz): δ 12.11 (bs, 1H), 10.3 (s, 1H), 8.06 (s, 1H), 7.9-7.84 (dd, 4H), 7.63 (d, 2H), 7.36 (d, 2H), 3.05 (s, 2H), 2.65 (t, 2H), 2.27 (s, 2H), 1.6 (m, 2H), 1.3 (m, 4H), 1.06 (s, 6H), 0.86 (t, 3H); MS: m/z 465 (M+1).

### Example 80

### Methyl 4-(5-(4-(2,4-dimethoxyphenylsulfonamido)phenyl)thiazol-2-yl)-3,3-dimethylbutanoate

The compound of example 80 was prepared analogous to the compound of example 24 by reaction of the compound of example 67 with 2,4-dimethoxybenzenesulfonyl chloride. Yield: 84%; ¹H NMR (DMSO-d₆, 300 MHz): δ 10.76 (s, 1H), 7.95 (s, 1H), 7.71 (d, 1H), 7.46 (d, 2H), 7.12 (d, 2H), 6.63 (d, 1H), 6.57 (dd, 1H), 3.86 (s, 3H), 3.78 (s, 3H), 3.58 (s, 3H), 2.99 (s, 2H), 2.27 (s, 2H), 1.02 (s, 6H); MS: m/z 505 (M+1).

### Example 81

### 4-(5-(4-(2,4-Dimethoxyphenylsulfonamido)phenyl)thiazol-2-yl)-3,3-dimethylbutanoic acid

The compound of example 81 was prepared analogous to the compound of example 25 by hydrolysis of the compound of example 80. Yield: 72%; ¹H NMR (DMSO-d₆, 300 MHz): δ 12.07 (bs, 1H), 10.07 (s, 1H), 7.95 (s, 1H), 7.71 (d, 1H), 7.46 (d, 2H), 7.12 (d, 2H), 6.62 (d, 1H), 6.57 (dd, 1H), 3.86 (s, 3H), 3.78 (s, 3H), 3.0 (s, 2H), 2.27 (s, 2H), 1.02 (s, 6H); MS: m/z 491 (M+1).

### Example 82

### Dimethyl 2,2-dimethylpentanedioate

3,3-dimethyldihydro-2H-pyran-2,6(3H)-dione (1.0 g) was dissolved in dry methanol (20 mL). To this solution, 1 drop of concentrated sulfuric acid was added and the reaction mixture was heated at 55° C. for 24 h. The reaction mixture was cooled, the solvent was removed and the residue was purified by column chromatography (silicagel, 20% ethyl acetate in petroleum ether) to afford the title compound. Yield: 1.12 (84%); ¹H NMR (DMSO-d₆, 300 MHz): δ 3.58 (s, 3H), 3.57 (s, 3H), 2.23 (m, 2H), 1.76 (m, 2H), 1.2 (s, 6H); MS: m/z 189 (M+1).

### Example 83

### 5-Methoxy-4,4-dimethyl-5-oxopentanoic acid

A mixture of the compound of example 83 (1.1 g), potassium carbonate (1.61 g), methanol (11 mL), tetrahydrofuran (6.6 mL) and water (6.6 mL) was stirred at room temperature for 48 h. The organic solvent was removed to obtain a residue, which was poured into water and extracted with ethyl acetate. The aqueous layer was acidified with 3N HCl and extracted with ethyl acetate. The organic layer obtained was washed with brine, dried over anhydrous sodium sulphate and evaporated to afford the title compound. Yield: 850 mg (83%); ¹H NMR (DMSO-d₆, 300 MHz): δ 12.1 (bs, 1H), 3.59 (s, 3H), 2.13 (m, 2H), 1.73 (m, 2H), 1.1 (s, 6H); MS: m/z 173 (M−1).

### Example 84

### Methyl 2,2-dimethyl-5-(2-(4-nitrophenyl)-2-oxoethylamino)-5-oxopentanoate

The compound of example 84 was prepared analogous to the compound of example 26 by reaction of the compound of example 2 with the compound of example 83. Yield 12.7 g (77%); ¹H NMR (DMSO-d₆, 300 MHz): δ 8.36 (t, 1H), 8.31 (d, 2H), 8.21 (d, 2H), 4.64 (d, 2H), 3.61 (s, 3H), 2.12 (m, 2H), 1.72 (m, 2H), 1.11 (s, 6H); MS: m/z 335 (M−1).

### Example 85

### Methyl 2,2-dimethyl-4-(5-(4-nitrophenyl)thiazol-2-yl)butanoate

The compound of example 85 was prepared analogous to the compound of example 4 by reaction of the compound of example 84 with Lawesson's reagent. Yield: 77%; ¹H NMR (CDCl₃, 300 MHz): δ 8.29 (d, 2H), 7.99 (s, 1H), 7.67 (d, 2H), 3.72 (s, 3H), 3.04 (m, 2H), 2.12 (m, 2H), 1.30 (s, 6H); MS: m/z 335 (M+1).

### Example 86

### Methyl 4-(5-(4-aminophenyl)thiazol-2-yl)-2,2-dimethylbutanoate

The compound of example 86 was prepared analogous to the compound of example 5 by reduction of the compound of example 85. Yield: 82%; ¹H NMR (DMSO-d₆, 300 MHz): δ 7.72 (s, 1H), 7.27 (d, 2H), 6.59 (d, 2H), 5.38 (bs, 2H), 3.62 (s, 3H), 2.85 (m, 2H), 1.95 (m, 2H), 1.19 (s, 6H); MS: m/z 305 (M+1).

### Example 87

### Methyl 2,2-dimethyl-4-(5-(4-(3-(3-(trifluoromethyl)phenyl)ureido)phenyl)thiazol-2-yl)butanoate

The compound of example 87 was prepared analogous to the compound of example 6 by reaction of the compound of example 86 with 1-isocyanato-3-trifluoromethyl benzene. Yield: 71%; ¹H NMR (DMSO-d₆, 300 MHz): δ 9.08 (s, 1H), 8.96 (s, 1H), 8.02 (d, 1H), 7.95 (s, 1H), 7.6-7.49 (dd, 6H), 7.33 (dd, 1H), 3.62 (s, 3H), 2.90 (m, 2H), 1.98 (m, 2H), 1.2 (s, 6H); MS: m/z 492 (M+1).

### Example 88

### 2,2-Dimethyl-4-(5-(4-(3-(3-(trifluoromethyl)phenyl)ureido)phenyl)thiazol-2-yl)butanoic acid

The compound of example 88 was prepared analogous to the compound of example 7 by hydrolysis of the compound of example 87. Yield: 63%; ¹H NMR (DMSO-d₆, 300 MHz): δ 12.29 (bs, 1H), 9.22 (s, 1H), 9.11 (s, 1H), 8.03 (d, 1H), 7.95 (s, 1H), 7.61-7.49 (dd, 6H), 7.33 (dd, 1H), 2.92 (m, 2H), 1.94 (m, 2H), 1.17 (s, 6H); MS: m/z 478 (M+1).

### Example 89

### Methyl 4-(5-(4-(3-(2-chlorophenyl)ureido)phenyl)thiazol-2-yl)-2,2-dimethyl butanoate

The compound of example 89 was prepared analogous to the compound of example 6 by reaction of the compound of example 86 with 1-chloro-2-isocyanato benzene. Yield: 80%; ¹H NMR (DMSO-d₆, 300 MHz): δ 9.57 (s, 1H), 8.34 (s, 1H), 8.18 (dd, 1H), 7.95 (s, 1H), 7.58-7.54 (dd, 4H), 7.48 (dd, 1H), 7.31 (m, 1H), 7.04 (m, 1H), 3.62 (s, 3H), 2.9 (m, 2H), 1.97 (m, 2H), 1.2 (s, 6H); MS: m/z 458 (M+1).

### Example 90

### 4-(5-(4-(3-(2-Chlorophenyl)ureido)phenyl)thiazol-2-yl)-2,2-dimethylbutanoic acid

The compound of example 90 was prepared analogous to the compound of example 7 by hydrolysis of the compound of example 89. Yield: 86%; ¹H NMR (DMSO-d₆, 300 MHz): δ 12.3 (bs, 1H), 9.58 (s, 1H), 8.35 (s, 1H), 8.18 (dd, 1H), 7.95 (s, 1H), 7.58-7.54 (d, 4H), 7.48 (dd, 1H), 7.31 (m, 1H), 7.04 (m, 1H), 2.92 (m, 2H), 1.95 (m, 2H), 1.17 (s, 6H); MS: m/z 444 (M+1).

### Example 90A

### Sodium salt of 4-(5-(4-(3-(2-chlorophenyl)ureido)phenyl)thiazol-2-yl)-2,2-dimethylbutanoic acid

To a solution of the compound of example 90 (100 mg) in THF (5 mL), 1N aqueous NaOH solution (9.01 mg, 0.224 mL) was added and reaction mixture was stirred for 1 h at room temperature. The solvent was removed and the residue obtained was triturated with ether, filtered and dried to afford the title compound.

Yield: 85 mg (80%); ¹H NMR (DMSO-d₆, 300 MHz): δ 12.38 (s, 1H), 10.88 (s, 1H), 7.88 (s, 1H), 7.78 (d, 2H), 7.71 (d, 1H), 7.53 (d, 2H), 7.43 (dd, 1H), 7.28 (m, 1H), 7.08 (m, 1H), 2.94 (m, 2H), 1.87 (m, 2H), 1.08 (s, 6H); MS (ES+): m/z 444.1 (M+1).

### Example 90B

### Potassium salt of 4-(5-(4-(3-(2-chlorophenyl)ureido)phenyl)thiazol-2-yl)-2,2-dimethylbutanoic acid

The compound of example 90B was prepared analogous to the compound of example 90A by reaction of the compound of example 90 with 1N KOH solution.

Yield: 94%; ¹H NMR (DMSO-d₆, 300 MHz): δ 12.73 (s, 1H), 11.21 (s, 1H), 7.88 (s, 1H), 7.81 (d, 2H), 7.68 (d, 1H), 7.53 (d, 2H), 7.43 (dd, 1H), 7.27 (m, 1H), 7.08 (m, 1H), 2.94 (m, 2H), 1.88 (m, 2H), 1.08 (s, 6H); MS (ES+): m/z 444.1 (M+1).

### Example 91

### Methyl 4-(5-(4-(3-(4-chloro-2-phenoxyphenyl)ureido)phenyl)thiazol-2-yl)-2,2-dimethylbutanoate

The compound of example 91 was prepared analogous to the compound of example 6 by reaction of the compound of example 86 with 4-chloro-1-isocyanato-2-phenoxy benzene. Yield: 80%; ¹H NMR (DMSO-d₆, 300 MHz): δ 9.5 (s, 1H), 8.69 (s, 1H), 8.4 (d, 1H), 7.94 (s, 1H), 7.54-7.51 (dd, 4H), 7.44 (dd, 2H), 7.22 (t, 1H), 7.1-7.08 (dd, 2H), 7.02-6.98 (dd, 1H), 6.85-6.82 (dd, 1H), 3.62 (s, 3H), 2.90 (m, 2H), 1.94 (m, 2H), 1.23 (s, 6H); MS: m/z 550 (M+1).

### Example 92

### 4-(5-(4-(3-(4-Chloro-2-phenoxyphenyl)ureido)phenyl)thiazol-2-yl)-2,2-dimethylbutanoic acid

The compound of example 92 was prepared analogous to the compound of example 7 by hydrolysis of the compound of example 91. Yield: 78%; ¹H NMR (DMSO-d₆, 300 MHz): δ 12.38 (bs, 1H), 9.52 (s, 1H), 8.7 (s, 1H), 8.4 (d, 1H), 7.95 (s, 1H), 7.57-7.51 (dd, 4H), 7.47 (d, 2H), 7.2 (t, 1H), 7.11 (dd, 2H), 7.02 (dd, 1H), 6.85 (dd, 1H), 2.92 (m, 2H), 1.93 (m, 2H), 1.17 (s, 6H); MS: m/z 536 (M+1).

### Example 93

### Methyl 4-(5-(4-(3-cyclohexylureido)phenyl)thiazol-2-yl)-2,2-dimethyl butanoate

The compound of example 93 was prepared analogous to the compound of example 6 by reaction of the compound of example 86 with isocyanato cyclohexane.

Yield: 63%; ¹H NMR (DMSO-d₆, 300 MHz): δ 8.45 (s, 1H), 7.88 (s, 1H), 7.45 (dd, 4H), 6.12 (d, 1H), 3.61 (s, 3H), 3.45 (m, 1H), 2.88 (m, 2H), 1.96 (m, 2H), 1.81 (m, 3H), 1.64 (m, 3H), 1.55 (m, 1H), 1.32 (m, 3H), 1.19 (s, 6H); MS: m/z 430 (M+1).

### Example 94

### 4-(5-(4-(3-Cyclohexylureido)phenyl)thiazol-2-yl)-2,2-dimethyl butanoic acid

The compound of example 94 was prepared analogous to the compound of example 7 by hydrolysis of the compound of example 93. Yield: 79%; ¹H NMR (DMSO-d₆, 300 MHz): δ 12.3 (bs, 1H), δ 8.48 (s, 1H), 7.8 (s, 1H), 7.48 (dd, 4H), 6.14 (d, 1H), 3.45 (m, 1H), 2.9 (m, 2H), 1.92 (m, 2H), 1.81 (m, 3H), 1.64 (m, 3H), 1.55 (m, 1H), 1.33 (m, 3H), 1.16 (s, 6H); MS: m/z 416 (M+1).

### Example 95

### Methyl 4-(5-(4-(3-(4-fluorophenyl)ureido)phenyl)thiazol-2-yl-2,2-dimethyl butanoate

The compound of example 95 was prepared analogous to the compound of example 6 by reaction of the compound of example 86 with 1-fluoro-4-isocyanato benzene.

Yield: 69%; ¹H NMR (DMSO-d₆, 300 MHz): δ 8.83 (s, 1H), 8.74 (s, 1H), 7.93 (s, 1H), 7.55-7.51 (dd, 4H), 7.46 (d, 2H), 7.15 (t, 2H), 3.62 (s, 3H), 2.89 (m, 2H), 1.98 (m, 2H), 1.2 (s, 6H); MS: m/z 442 (M+1).

### Example 96

### Methyl 4-(5-(4-(3-(4-fluorophenyl)ureido)phenyl)thiazol-2-yl-2,2-dimethyl butanoate

The compound of example 96 was prepared analogous to the compound of example 7 by hydrolysis of the compound of example 95. Yield: 66%; ¹H NMR (DMSO-d₆, 300 MHz): δ 12.3 (bs, 1H), 8.84 (s, 1H), 8.75 (s, 1H), 7.93 (s, 1H), 7.55-7.51 (dd, 4H), 7.46 (d, 2H), 7.12 (t, 2H), 2.91 (m, 2H), 1.94 (m, 2H), 1.17 (s, 6H); MS: m/z 428 (M+1).

### Example 97

### Methyl 4-(5-(4-(3-(4-methoxyphenyl)ureido)phenyl)thiazol-2-yl)-2,2-dimethyl butanoate

The compound of example 97 was prepared analogous to the compound of example 6 by reaction of the compound of example 86 with 1-isocyanato-4-methoxy benzene.

Yield: 75%; ¹H NMR (DMSO-d₆, 300 MHz): δ 8.75 (s, 1H), 8.51 (s, 1H), 7.92 (s, 1H), 7.54-7.47 (dd, 4H), 7.37 (d, 2H), 6.88 (d, 2H), 3.71 (s, 3H), 3.62 (s, 3H), 2.89 (m, 2H), 1.97 (m, 2H), 1.2 (s, 6H); MS: m/z 454 (M+1).

### Example 98

### 4-(5-(4-(3-(4-Methoxyphenyl)ureido)phenyl)thiazol-2-yl)-2,2-dimethyl butanoic acid

The compound of example 98 was prepared analogous to the compound of example 7 by hydrolysis of the compound of example 97. Yield: 93%; ¹H NMR (DMSO-d₆, 300 MHz): δ 12.3 (bs, 1H), 8.77 (s, 1H), 8.53 (s, 1H), 7.93 (s, 1H), 7.54-7.48 (dd, 4H), 7.37 (d, 2H), 6.88 (d, 2H), 3.71 (s, 3H), 2.91 (m, 2H), 1.93 (m, 2H), 1.17 (s, 6H); MS: m/z 440 (M+1).

### Example 99

### Methyl 4-(5-(4-(3-(4-isopropylphenyl)ureido)phenyl)thiazol-2-yl)-2,2-dimethyl butanoate

The compound of example 99 was prepared analogous to the compound of example 6 by reaction of the compound of example 86 with 1-isocyanato-4-isopropyl benzene.

Yield: 73%; ¹H NMR (DMSO-d₆, 300 MHz): δ 8.78 (s, 1H), 8.6 (s, 1H), 7.93 (s, 1H), 7.51 (dd, 4H), 7.37 (d, 2H), 7.16 (d, 2H), 3.62 (s, 3H), 2.89 (m, 2H), 2.86 (m, 1H), 1.98 (m, 2H), 1.19 (s, 6H), 1.17 (d, 6H); MS: m/z 466 (M+1).

### Example 100

### 4-(5-(4-(3-(4-Isopropylphenyl)ureido)phenyl)thiazol-2-yl)-2,2-dimethyl butanoic acid

The compound of example 100 was prepared analogous to the compound of example 7 by hydrolysis of the compound of example 99. Yield: 65%; ¹H NMR (DMSO-d₆, 300 MHz): δ 8.93 (s, 1H), 8.73 (s, 1H), 7.94 (s, 1H), 7.52 (dd, 4H), 7.37 (d, 2H), 7.16 (d, 2H), 2.92 (m, 2H), 2.83 (m, 1H), 1.93 (m, 2H), 1.19 (s, 6H), 1.17, (d, 6H); MS: m/z 452 (M+1).

### Example 101

### Methyl 4-(5-(4-(3-(2,4-difluorophenyl)ureido)phenyl)thiazol-2-yl)-2,2-dimethylbutanoate

The compound of example 101 was prepared analogous to the compound of example 6 by reaction of the compound of example 86 with 2,4-difluoro-1-isocyanato benzene.

Yield: 79%; ¹H NMR (DMSO-d₆, 300 MHz): δ 9.17 (s, 1H), 8.53 (s, 1H), 8.12-8.03 (m, 1H), 7.94 (s, 1H), 7.56-7.52 (dd, 4H), 7.36-7.28 (m, 1H), 7.08-7.03 (m, 1H), 3.62 (s, 3H), 2.9 (m, 2H), 1.93 (m, 2H), 1.2 (s, 6H); MS: m/z 459 (M+1).

### Example 102

### 4-(5-(4-(3-(2,4-Difluorophenyl)ureido)phenyl)thiazol-2-yl)-2,2-dimethyl butanoic acid

The compound of example 102 was prepared analogous to the compound of example 7 by hydrolysis of the compound of example 101. Yield: 97%; ¹H NMR (DMSO-d₆, 300 MHz): δ 9.36 (s, 1H), 8.63 (s, 1H), 8.11-8.03 (m, 1H), 7.96 (s, 1H), 7.57-7.5 (dd, 4H), 7.36-7.28 (m, 1H), 7.09-7.03 (m, 1H), 2.93 (m, 2H), 1.94 (m, 2H), 1.17 (s, 6H); MS: m/z 446 (M+1).

### Example 102A

### Sodium salt of 4-(5-(4-(3-(2,4-difluorophenyl)ureido)phenyl)thiazol-2-yl)-2,2-dimethyl butanoate

The compound of example 102A is prepared analogous to the compound of example 90A by reaction of the compound of example 102 with 1N NaOH solution. Yield: 74%;

¹H NMR (DMSO-d₆, 300 MHz): δ 12.68 (s, 1H), 11.55 (s, 1H), 7.87 (s, 1H), 7.81-7.78 (d, 2H), 7.68-7.60 (m, 1H), 7.53-7.51 (d, 2H), 7.25-7.19 (m, 1H), 7.04-6.98 (m, 1H), 2.94 (m, 2H), 1.89 (m, 2H), 1.09 (s, 6H); MS: m/z 446 (M+1).

### Example 102B

### Potassium salt of 4-(5-(4-(3-(2,4-difluorophenyl)ureido)phenyl)thiazol-2-yl)-2,2-dimethylbutanoate

The compound of example 102B is prepared analogous to the compound of example 90A by reaction of the compound of example 102 with 1N KOH solution. Yield: 69%;

¹H NMR (DMSO-d₆, 300 MHz): δ 12.84 (s, 1H), 11.69 (s, 1H), 7.87 (s, 1H), 7.82-7.79 (d, 2H), 7.66-7.58 (m, 1H), 7.53-7.51 (d, 2H), 7.24-7.18 (m, 1H), 7.03-6.98 (m, 1H), 2.94 (m, 2H), 1.89 (m, 2H), 1.09 (s, 6H); MS: m/z 446 (M+1).

### Example 103

### Methyl 4-(5-(4-(3-(2-fluorophenyl)ureido)phenyl)thiazol-2-yl)-2,2-dimethyl butanoate

Methyl 4-(5-(4-aminophenyl)thiazol-2-yl)-2,2-dimethylbutanoate (200 mg) was dissolved in tetrahydrofuran (8 mL) to which was added 2-fluoroaniline (146 mg) and carbonyl diimidazole (266 mg) and the reaction mixture was stirred at room temperature for 24 h. The solvent was removed to obtain a residue, which was purified by column chromatography (silicagel, ethyl acetate in chloroform) to yield a solid, which was crystallized in methylene chloride in petroleum ether to afford the title compound. Yield: 155 mg (53%); ¹H NMR (DMSO-D6, 300 MHz) δ 9.22 (s, 1H), 8.57 (s, 1H), 8.14 (dd, 1H), 7.94 (s, 1H), 7.57-7.49 (dd, 4H), 7.27-7.21 (dd, 1H), 7.17-7.12 (m, 1H), 7.03 (m, 1H), 3.62 (s, 3H), 2.9 (m, 2H), 1.97 (m, 2H), 1.2 (s, 6H); MS: m/z 442 (M+1).

### Example 104

### 4-(5-(4-(3-(2-Fluorophenyl)ureido)phenyl)thiazol-2-yl)-2,2-dimethylbutanoic acid

The compound of example 104 was prepared analogous to the compound of example 7 by hydrolysis of the compound of example 103. Yield: 71%; ¹H NMR (DMSO-d₆, 300 MHz): δ 12.37 (bs, 1H), 9.24 (s, 1H), 8.59 (s, 1H), 8.15 (dd, 1H), 7.95 (s, 1H), 7.57-7.5 (dd, 4H), 7.28-7.21 (dd, 1H), 7.18-7.13 (m, 1H), 7.03 (m, 1H), 2.92 (m, 2H), 1.94 (m, 2H), 1.17 (s, 6H); MS: m/z 428 (M+1).

### Example 104A

### Sodium salt of 4-(5-(4-(3-(2-fluorophenyl)ureido)phenyl)thiazol-2-yl-2,2-dimethylbutanoate

The compound of example 104A is prepared analogous to the compound of example 90A by reaction of the compound of example 104 with 1N NaOH solution. Yield: 66%;

¹H NMR (DMSO-d₆, 300 MHz): δ 11.49 (s, 1H), 10.40 (s, 1H), 7.89 (s, 1H), 7.87-7.83 (m, 1H), 7.71-7.68 (d, 2H), 7.54-7.51 (d, 2H), 7.19-7.10 (m, 2H), 7.04-7.02 (m, 1H), 2.93 (m, 2H), 1.90 (m, 2H), 1.12 (s, 6H); MS: m/z 428.1 (M+1).

### Example 104B

### Potassium 4-(5-(4-(3-(2-fluorophenyl)ureido)phenyl)thiazol-2-yl)-2,2-dimethyl butanoate

The compound of example 104B is prepared analogous to the compound of example 90A by reaction of the compound of example 104 with 1N KOH solution. Yield: 76%;

¹H NMR (DMSO-d₆, 300 MHz): δ 12.41 (s, 1H), 11.23 (s, 1H), 7.88 (s, 1H), 7.79-7.77 (d, 2H), 7.74-7.72 (m, 1H), 7.53-7.51 (d, 2H), 7.20-7.12 (m, 2H), 7.09-7.05 (m, 1H), 2.94 (m, 2H), 1.90 (m, 2H), 1.10 (s, 6H); MS: m/z 428.1 (M+1).

### Example 105

### Methyl 4-(5-(4-(4-tert-butylbenzamido)phenyl)thiazol-2-yl)-2,2-dimethyl butanoate

The compound of example 105 was prepared analogous to the compound of example 14 by reaction of the compound of example 86 with 4-(t-butyl)benzoyl chloride. Yield: 65%; ¹H NMR (DMSO-d₆, 300 MHz): δ 10.31 (s, 1H), 8.0 (s, 1H), 7.91-7.84 (dd, 4H), 7.62-7.54 (dd, 4H), 3.62 (s, 3H), 2.91 (m, 2H), 1.98 (m, 2H), 1.32 (s, 9H), 1.2 (s, 6H); MS: m/z 465 (M+1).

### Example 106

### 4-(5-(4-(4-tert-Butyl benzamido)phenyl)thiazol-2-yl)-2,2-dimethylbutanoic acid

The compound of example 106 was prepared analogous to the compound of example 15 by hydrolysis of the compound of example 105. Yield: 36%; ¹H NMR (DMSO-d₆, 300 MHz): δ 12.31 (bs, 1H), 10.31 (s, 1H), 8.0 (s, 1H), 7.91-7.84 (dd, 4H), 7.63-7.54 (dd, 4H), 2.93 (m, 2H), 1.94 (m, 2H), 1.32 (s, 9H), 1.17 (s, 6H); MS: m/z 451 (M+1).

### Example 107

### Methyl 4-(5-(4-biphenyl-4-ylcarboxamidophenyl)thiazol-2-yl)-2,2-dimethyl butanoate

The compound of example 107 was prepared analogous to the compound of example 14 by reaction of the compound of example 86 with 4-phenyl benzoyl chloride. Yield: 31%; ¹H NMR (DMSO-d₆, 300 MHz): δ 10.43 (s, 1H), 8.09 (d, 2H), 8.0 (s, 1H), 7.9-7.84 (dd, 4H), 7.78 (dd, 2H), 7.64 (dd, 2H), 7.52 (dd, 2H), 7.45 (dd, 1H), 3.63 (s, 3H), 2.91 (m, 2H), 1.98 (m, 2H), 1.2 (s, 6H); MS: m/z 485 (M+1).

### Example 108

### 4-(5-(4-Biphenyl-4-ylcarboxamidophenyl)thiazol-2-yl)-2,2-dimethylbutanoic acid

The compound of example 108 was prepared analogous to the compound of example 15 by hydrolysis of the compound of example 107. Yield: 95%; ¹H NMR (DMSO-d₆, 300 MHz): δ 10.67 (s, 1H), 8.13 (d, 2H), 7.96 (s, 1H), 7.91 (d, 2H), 7.85 (d, 2H), 7.77 (dd, 2H), 7.61 (d, 2H), 7.51 (dd, 2H), 7.45 (dd, 1H), 2.92 (m, 2H), 1.82 (m, 2H), 1.05 (s, 6H); MS: m/z 471 (M+1).

### Example 109

### Methyl 2,2-dimethyl-4-(5-(4-(4-(oxazol-5-yl)benzamido)phenyl)thiazol-2-yl)butanoate

To a solution of the compound of example 86 (150 mg) and methyl 4-(oxazol-5-yl)benzoate (120 mg) in toluene (12 mL) was added a solution of trimethyl aluminium (0.38 mL, 2M solution in toluene). The mixture was sealed and heated at 80° C. for 4 h. The reaction mixture was cooled to room temperature, water was added and the reaction mixture was neutralized with saturated aqueous solution of sodium carbonate. The reaction mixture was extracted with ethyl acetate and the layers were separated. The organic layer was washed with brine solution, dried over anhydrous sodium sulphate and the solvent was evaporated to obtain a residue, which was purified by column chromatography (silicagel, ethyl acetate in petroleum ether) to yield a solid. The solid was crystallized in chloroform in petroleum ether to afford the title compound.

Yield: 184 mg (78%); ¹H NMR (DMSO-d₆, 300 MHz): δ 10.44 (s, 1H), 8.5 (s, 1H), 8.1 (d, 2H), 8.0 (s, 1H), 7.91-7.85 (ddd, 5H), 7.64 (d, 2H), 3.62 (s, 3H), 2.92 (m, 2H), 1.98 (m, 2H), 1.2 (s, 6H); MS: m/z 476 (M+1).

### Example 110

### 2,2-Dimethyl-4-(5-(4-(4-(oxazol-5-yl)benzamido)phenyl)thiazol-2-yl)butanoic acid

The compound of example 110 was prepared analogous to the compound of example 15 by hydrolysis of the compound of example 109. Yield: 75%; ¹H NMR (DMSO-d₆, 300 MHz): δ 10.69 (s, 1H), 8.53 (s, 1H), 8.14 (d, 2H), 7.96 (s, 1H), 7.89-7.58 (ddd, 5H), 7.61 (d, 2H), 2.91 (m, 2H), 1.82 (m, 2H), 1.05 (s, 6H); MS: m/z 462 (M+1).

### Example 111

### Methyl 2,2-dimethyl-4-(5-(4-(4-phenylthiazole-2-carboxamido)phenyl)thiazol-2-yl) butanoate

The compound of example 111 was prepared analogous to the compound of example 109 by reaction of the compound of example 86 with 4-phenyl-thiazole-2-carbonyl chloride. Yield: 55%; ¹H NMR (DMSO-d₆, 300 MHz): δ 10.75 (s, 1H), 8.52 (s, 1H), 8.19 (d, 2H), 8.03 (s, 1H), 7.97 (d, 2H), 7.68 (d, 2H), 7.52 (dd, 2H), 7.42 (dd, 1H), 3.62 (s, 3H), 2.92 (m, 2H), 1.98 (m, 2H), 1.2 (s, 6H); MS: m/z 492 (M+1).

### Example 112

### 2,2-Dimethyl-4-(5-(4-(4-phenylthiazole-2-carboxamido)phenyl)thiazol-2-yl)butanoic acid

The compound of example 112 was prepared analogous to the compound of example 15 by hydrolysis of the compound of example 111. Yield: 62%; ¹H NMR (DMSO-d₆, 300 MHz): δ 12.31 (bs, 1H), 10.75 (s, 1H), 8.52 (s, 1H), 8.47 (s, 1H), 8.19 (d, 1H), 8.0-7.94 (dd, 2H), 7.68 (d, 1H), 7.54-7.37 (dd, 4H), 7.27 (d, 1H), 2.91 (m, 2H), 1.95 (m, 2H), 1.17 (s, 6H); MS: m/z 478 (M+1).

### Example 113

### Methyl 2,2-dimethyl-3-(5-(4-nitrophenyl)oxazol-2-yl)propanoate

A solution of the compound of example 26 (4.2 g) in phosophorous oxychloride (21 mL) was refluxed at 106 to 108° C. for 6 h. The reaction mixture was quenched in ice, neutralized with sodium carbonate and extracted with methylene chloride. The organic layer was separated, dried over anhydrous sodium sulphate and concentrated to obtain a residue. The residue was purified by column chromatography (silicagel, 30% ethyl acetate in petroleum ether) to obtain a solid, which was crystallized in ethyl acetate in petroleum ether to afford the title compound. Yield: 56%; ¹H NMR (CDCl₃, 300 MHz): δ 8.31 (d, 2H), 7.75 (d, 2H), 7.45 (s, 1H), 3.75 (s, 3H), 3.16 (s, 2H), 1.35 (s, 6H); MS: m/z 305 (M+1).

### Example 114

### Methyl 3-(5-(4-aminophenyl)oxazol-2-yl)-2,2-dimethylpropanoate

The compound of example 114 was prepared analogous to the compound of example 5 by reduction of the compound of example 113. Yield: 78%; ¹H NMR (DMSO-d₆, 300 MHz): δ 7.29 (d, 2H), 7.15 (s, 1H), 6.61 (d, 2H), 5.41 (bs, 2H), 3.62 (s, 3H), 2.99 (s, 2H), 1.21 (s, 6H); MS: m/z 275 (M+1).

### Example 115

### Methyl 3-(5-(4-(3-(2-chlorophenyl)ureido)phenyl)oxazol-2-yl)-2,2-dimethyl propanoate

The compound of example 115 was prepared analogous to the compound of example 6 by reaction of the compound of example 114 with 1-chloro-2-isocyanato benzene.

Yield: 64%; ¹H NMR (DMSO-d₆, 300 MHz): δ 9.58 (s, 1H), 8.35 (s, 1H), 8.17 (dd, 1H), 7.56 (dd, 4H), 7.48 (dd, 1H), 7.42 (s, 1H) 7.31 (m, 1H), 7.04 (m, 1H), 3.64 (s, 3H), 3.05 (s, 2H), 1.24 (s, 6H); MS: m/z 428 (M+1).

### Example 116

### 3-(5-(4-(3-(2-Chlorophenyl)ureido)phenyl)oxazol-2-yl)-2,2-dimethyl propanoic acid

The compound of example 116 was prepared analogous to the compound of example 7 by hydrolysis of the compound of example 115. Yield: 86%; ¹H NMR (DMSO-d₆, 300 MHz): δ 12.41 (bs, 1H), 9.6 (s, 1H), 8.35 (s, 1H), 8.17 (dd, 1H), 7.57 (dd, 4H), 7.48 (dd, 1H), 7.42 (s, 1H), 7.31 (m, 1H), 7.04 (m, 1H), 3.01 (s, 2H), 1.21 (s, 6H); MS: m/z 414 (M+1).

### Example 117

### Methyl 2,2-dimethyl-3-(5-(4-(3-(4-(trifluoromethyl)phenyl)ureido)phenyl)oxazol-2-yl)propanoate

The compound of example 117 was prepared analogous to the compound of example 6 by reaction of the compound of example 114 with 1-isocyanato-4-(trifluoromethyl)benzene.

Yield: 89%; ¹H NMR (DMSO-d₆, 300 MHz): δ 9.14 (s, 1H), 8.99 (s, 1H), 7.66 (dd, 4H), 7.56 (dd, 4H), 7.42 (s, 1H), 3.64 (s, 3H), 3.05 (s, 2H), 1.23 (s, 6H); MS: m/z 462 (M+1).

### Example 118

### 2,2-Dimethyl-3-(5-(4-(3-(4-(trifluoromethyl)phenyl)ureido)phenyl)oxazol-2-yl)propanoic acid

The compound of example 118 was prepared analogous to the compound of example 7 by hydrolysis of the compound of example 117. Yield: 94%; ¹H NMR (DMSO-d₆, 300 MHz): δ 12.39 (bs, 1H), 9.44 (s, 1H), 9.27 (s, 1H), 7.66 (dd, 4H), 7.6 (dd, 4H), 7.41 (s, 1H), 3.01 (s, 2H), 1.21 (s, 6H); MS: m/z 448 (M+1).

### Example 119

### Methyl 3-(5-(4-(3-(4-fluorophenyl)ureido)phenyl)oxazol-2-yl)-2,2-dimethyl propanoate

The compound of example 119 was prepared analogous to the compound of example 6 by reaction of the compound of example 114 with 1-isocyanato-4-fluoro benzene.

Yield: 68%; ¹H NMR (DMSO-d₆, 300 MHz): δ 8.85 (s, 1H), 8.74 (s, 1H), 7.54 (dd, 4H), 7.46 (d, 2H), 7.4 (s, 1H), 7.12 (d, 2H), 3.64 (s, 3H), 3.04 (s, 2H), 1.23 (s, 6H); MS: m/z 412 (M+1).

### Example 120

### 3-(5-(4-(3-(4-Fluorophenyl)ureido)phenyl)oxazol-2-yl)-2,2-dimethylpropanoic acid

The compound of example 120 was prepared analogous to the compound of example 7 by hydrolysis of the compound of example 119. Yield: 77%; ¹H NMR (DMSO-d₆, 300 MHz): δ 12.41 (bs, 1H), 8.87 (s, 1H), 8.77 (s, 1H), 7.54 (dd, 4H), 7.46 (d, 2H), 7.4 (s, 1H), 7.12 (d, 2H), 3.0 (s, 2H), 1.21 (s, 6H); MS: m/z 398 (M+1).

### Example 121

### Methyl 3-(5-(4-(3-(4-methoxyphenyl)ureido)phenyl)oxazol-2-yl)-2,2-dimethyl propanoate

The compound of example 121 was prepared analogous to the compound of example 6 by reaction of the compound of example 114 with 1-isocyanato-4-methoxy benzene.

Yield: 64%; ¹H NMR (DMSO-d₆, 300 MHz): δ 8.76 (s, 1H), 8.51 (s, 1H), 7.53 (dd, 4H), 7.39 (s, 1H), 7.37 (d, 2H), 6.88 (d, 2H), 3.71 (s, 3H), 3.63 (s, 3H), 3.04 (s, 2H), 1.23 (s, 6H); MS: m/z 424 (M+1).

### Example 122

### 3-(5-(4-(3-(4-Methoxyphenyl)ureido)phenyl)oxazol-2-yl)-2,2-dimethyl propanoic acid

The compound of example 122 was prepared analogous to the compound of example 7 by hydrolysis of the compound of example 121. Yield: 93%; ¹H NMR (DMSO-d₆, 300 MHz): δ 12.37 (bs, 1H), 8.88 (s, 1H), 8.62 (s, 1H), 7.54 (dd, 4H), 7.39 (s, 1H), 7.37 (d, 2H), 6.88 (d, 2H), 3.71 (s, 3H), 3.0 (s, 2H), 1.21 (s, 6H); MS: m/z 410 (M+1).

### Example 123

### Methyl 3-(5-(4-(3-(4-chloro-2-phenoxyphenyl)ureido)phenyl)oxazol-2-yl)-2,2-dimethylpropanoate

The compound of example 123 was prepared analogous to the compound of example 6 by reaction of the compound of example 114 with 4-chloro-1-isocyanato-2-phenoxy benzene. Yield: 81%; ¹H NMR (DMSO-d₆, 300 MHz): δ 9.52 (s, 1H), 8.71 (s, 1H), 8.39 (d, 1H), 7.58-7.54 (dd, 4H), 7.44 (dd, 2H), 7.41 (s, 1H), 7.2 (t, 1H), 7.1 (dd, 2H), 7.02-6.98 (dd, 1H), 6.85-6.82 (dd, 1H), 3.63 (s, 3H), 3.04 (s, 2H), 1.23 (s, 6H); MS: m/z 520 (M+1).

### Example 124

### 3-(5-(4-(3-(4-Chloro-2-phenoxyphenyl)ureido)phenyl)oxazol-2-yl)-2,2-dimethyl propanoic acid

The compound of example 124 was prepared analogous to the compound of example 7 by hydrolysis of the compound of example 123. Yield: 86%; ¹H NMR (DMSO-d₆, 300 MHz): δ 9.56 (s, 1H), 8.72 (s, 1H), 8.39 (d, 1H), 7.59-7.51 (dd, 4H), 7.44 (dd, 2H), 7.42 (s, 1H), 7.19 (t, 1H), 7.1 (dd, 2H), 7.02-6.98 (dd, 1H), 6.85-6.82 (dd, 1H), 3.0 (s, 2H), 1.21 (s, 6H); MS: m/z 506 (M+1).

### Example 125

### Methyl 3-(5-(4-(4-tert-butylbenzamido)phenyl)oxazol-2-yl)-2,2-dimethyl propanoate

The compound of example 125 was prepared analogous to the compound of example 14 by reaction of the compound of example 114 with 4-(t-butyl)benzoyl chloride. Yield: 94%; ¹H NMR (DMSO-d₆, 300 MHz): δ 10.34 (s, 1H), 8.01-7.96 (dd, 4H), 7.92-7.88 (dd, 4H), 7.47 (s, 1H), 3.64 (s, 3H), 3.05 (s, 2H), 1.32 (s, 9H), 1.24 (s, 6H); MS: m/z 435 (M+1).

### Example 126

### 3-(5-(4-(4-Tert-butyl benzamido)phenyl)oxazol-2-yl)-2,2-dimethylpropanoic acid

The compound of example 126 was prepared analogous to the compound of example 15 by hydrolysis of the compound of example 125. Yield: 85%; ¹H NMR (DMSO-d₆, 300 MHz): δ 12.43 (bs, 1H), 10.32 (s, 1H), 7.91-7.87 (dd, 4H), 7.65 (d, 2H), 7.57 (d, 2H), 7.47 (s, 1H), 3.02 (s, 2H), 1.32 (s, 9H), 1.22 (s, 6H); MS: m/z 437 (M+1).

### Example 127

### Methyl 3-(5-(4-biphenyl-4-ylcarboxamidophenyl)oxazol-2-yl)-2,2-dimethyl propanoate

The compound of example 127 was prepared analogous to the compound of example 14 by reaction of the compound of example 114 with 4-phenyl benzoyl chloride. Yield: 91%; ¹H NMR (DMSO-d₆, 300 MHz): δ 10.45 (s, 1H), 8.09 (d, 2H), 7.97-7.91 (dd, 2H), 7.86 (dd, 2H), 7.78 (dd, 2H), 7.65 (dd, 2H), 7.52 (dd, 2H), 7.48 (s, 1H), 7.43 (dd, 1H), 3.74 (s, 3H), 3.06 (s, 2H), 1.25 (s, 6H); MS: m/z 455 (M+1).

### Example 128

### 3-(5-(4-Biphenyl-4-ylcarboxamidophenyl)oxazol-2-yl)-2,2-dimethylpropanoic acid

The compound of example 128 was prepared analogous to the compound of example 15 by hydrolysis of the compound of example 127. Yield: 88%; ¹H NMR (DMSO-d₆, 300 MHz): δ 12.41 (bs, 1H), 10.45 (s, 1H), 8.07 (d, 2H), 7.94 (d, 2H), 7.87 (dd, 2H), 7.78 (d, 2H), 7.67 (d, 2H), 7.52 (dd, 2H), 7.48 (s, 1H), 7.43 (dd, 1H), 3.03 (s, 2H), 1.22 (s, 6H); MS: m/z 441 (M+1).

### Example 129

### trans-4-(Methoxycarbonyl)cyclohexanecarboxylic acid

The compound of example 129 was prepared according to the procedure described in Journal of Medicinal Chemistry, Eng, 2004, 47, 9, 2318-25.

Dimethyl trans-1,4-cyclohexanedicarboxylate (1g) was dissolved in methanol (12 mL) and heated to reflux for 10-15 min. KOH (0.329 g) in methanol (5 mL) was added dropwise and the reaction mixture was stirred under reflux for 5 h. The reaction mixture was cooled to room temperature and concentrated to dryness. Water was added and dilute HCl solution was added till a solid was precipitated. The solid was filtered and washed with water. The solid was dried to afford the title compound. Yield: 0.550 g (58%); ¹H NMR (DMSO-d₆, 300 MHz): δ 12.07 (bs, 1H), 3.58 (s, 3H), 2.30 (m, 1H), 2.16 (m, 1H), 1.9 (m, 4H), 1.37 (m, 4H); MS: m/z 185 (M−1).

### Example 130

### Methyl 4-(2-(4-nitrophenyl)-2-oxoethylcarbamoyl)cyclohexanecarboxylate

To the compound of example 129 (15 g) in DMF (120 mL) was added the compound of example 2 (20.95 g), BOP reagent (39 g) and triethylamine (22.4 mL) and the reaction mixture was stirred at 60° C. for about 16 h. The reaction mixture was cooled to room temperature, water and ethyl acetate was added and the reaction mixture was stirred. The organic layer was separated and washed with dilute HCl, sodium bicarbonate solution and water. The organic solvent was removed to obtain a residue, which was purified by column chromatography (silicagel, EtOAc in chloroform) to afford the title compound. Yield: 12 g (42%); ¹H NMR (DMSO-d₆, 300 MHz): δ 8.36 (d, 2H), 8.22 (t, 1H), 8.20 (d, 2H), 4.61 (d, 2H), 3.59 (s, 3H), 2.28 (m, 2H), 1.94 (m, 2H), 1.80 (m, 2H), 1.40 (m, 4H); MS: m/z 349 (M+1), 371 (M+Na).

### Example 131

### Methyl 4-(5-(4-nitrophenyl)thiazol-2-yl)cyclohexanecarboxylate

The compound of example 131 was prepared analogous to the compound of example 4 by reaction of the compound of example 130 with Lawesson's reagent at 60° C. for about 5 h. Yield: 52%; ¹H NMR (DMSO-d₆, 300 MHz): δ 8.35 (s, 1H), 8.28 (d, 2H), 7.93 (d, 2H), 3.61 (s, 3H), 3.10 (m, 1H), 2.45 (m, 1H), 2.18 (m, 2H), 2.04 (m, 2H), 1.61 (m, 4H); MS: m/z 347.1 (M+1).

### Example 132

### Methyl 4-(5-(4-aminophenyl)thiazol-2-yl)cyclohexanecarboxylate

The compound of example 132 was prepared analogous to the compound of example 5 by reduction of the compound of example 131. Yield: 71%; ¹H NMR (DMSO-d₆, 300 MHz): δ 7.73 (5, 1H), 7.27 (d, 2H), 6.59 (d, 2H), 5.37 (s, 2H), 3.61 (s, 3H), 2.96 (m, 1H), 2.43 (m, 1H), 2.13 (m, 2H), 2.01 (m, 2H), 1.55 (m, 4H); MS: m/z 317.1 (M+1).

### Example 133

### Methyl 4-(5-(4-(3-(3-(trifluoromethyl)phenyl)ureido)phenyl)thiazol-2-yl)cyclohexanecarboxylate

The compound of example 133 was prepared analogous to the compound of example by reaction of the compound of example 132 with 1-isocyanato-3-(trifluoromethyl)benzene. The solvent was removed to obtain a solid, which was crystallized using acetone in petroleum ether to afford the title compound.

Yield: 87%; ¹H NMR (DMSO-d₆, 300 MHz): δ 9.09 (s, 1H), 8.97 (5, 1H), 8.01 (5, 1H), 7.96 (s, 1H), 7.60 (m, 6H), 7.33 (d, 1H), 3.61 (5, 3H), 2.97 (m, 1H), 2.41 (m, 1H), 2.16 (m, 2H), 2.03 (m, 2H), 1.58 (m, 4H); MS: m/z 504.1 (M+1).

### Example 134

### 4-(5-(4-(3-(3-(Trifluoromethyl)phenyl)ureido)phenyl)thiazol-2-yl)cyclohexane carboxylic acid

The compound of example 134 was prepared analogous to the compound of example 7 by hydrolysis of the compound of example 133. The crude product obtained was crystallized using acetone and petroleum ether to afford the title compound. Yield: 64%; ¹H NMR (DMSO-d₆, 300 MHz): δ 9.10 (s, 1H), 8.98 (s, 1H), 8.01 (s, 1H), 7.95 (s, 1H), 7.57 (m, 6H), 7.33 (d, 1H), 2.95 (m, 1H), 2.22 (m, 1H), 2.15 (m, 2H), 2.02 (m, 2H), 1.56 (m, 4H); MS: m/z 490.2 (M+1).

### Example 135

### Methyl 4-(5-(4-(3-p-tolylureido)phenyl)thiazol-2-yl)cyclohexanecarboxylate

The compound of example 135 was prepared analogous to the compound of example 6 by reaction of the compound of example 134 with 1-isocyanato-4-methylbenzene.

Yield: 42%; ¹H NMR (DMSO-d₆, 300 MHz): δ 8.78 (s, 1H), 8.58 (s, 1H), 7.94 (s, 1H), 7.55 (m, 4H), 7.35 (d, 2H), 7.10 (d, 2H), 3.61 (s, 3H), 2.97 (m, 1H), 2.42 (m, 1H), 2.24 (s, 3H), 2.16 (m, 2H), 2.03 (m, 2H), 1.58 (m, 4H); MS: m/z 448 (M−1).

### Example 136

### 4-(5-(4-(3-p-Tolylureido)phenyl)thiazol-2-yl)cyclohexanecarboxylic acid

The compound of example 136 was prepared analogous to the compound of example 7 by hydrolysis of the compound of example 135. Yield: 21%; ¹H NMR (DMSO-d₆, 300 MHz): δ 9.01 (s, 1H), 8.80 (s, 1H), 7.96 (s, 1H), 7.52 (m, 4H), 7.35 (d, 2H), 7.10 (d, 2H), 2.96 (m, 1H), 2.39 (m, 1H), 2.24 (s, 3H), 2.12 (m, 2H), 2.03 (m, 2H), 1.61 (m, 4H); MS: m/z 436 (M+1).

### Example 137

### Methyl 4-(5-(4-(3-(2,4-difluorophenyl)ureido)phenyl)thiazol-2-yl)cyclohexane carboxylate

The compound of example 137 was prepared analogous to the compound of example 6 by reaction of the compound of example 132 with 1-isocyanato-2,4-difluorobenzene.

Yield: 41%; ¹H NMR (DMSO-d₆, 300 MHz): δ 9.16 (s, 1H), 8.53 (s, 1H), 8.12 (m, 1H), 7.95 (s, 1H), 7.55 (m, 4H), 7.35 (t, 1H), 7.08 (t, 1H), 3.61 (s, 3H), 2.99 (m, 1H), 2.42 (m, 1H), 2.15 (m, 2H), 2.03 (m, 2H), 1.58 (m, 4H); MS: m/z 472 (M+1); m/z 470 (M−1).

### Example 138

### 4-(5-(4-(3-(2,4-Difluorophenyl)ureido)phenyl)thiazol-2-yl)cyclohexane carboxylic acid

The compound of example 138 was prepared analogous to the compound of example 7 by hydrolysis of the compound of example 137. Yield: 70%; ¹H NMR (DMSO-d₆, 300 MHz): δ 12.13 (s, 1H), 9.21 (s, 1H), 8.55 (s, 1H), 8.12 (m, 1H), 7.96 (s, 1H), 7.57 (m, 4H), 7.36 (t, 1H), 7.09 (t, 1H), 2.98 (m, 1H), 2.28 (m, 1H), 2.16 (m, 2H), 2.03 (m, 2H), 1.61 (m, 4H); MS: m/z 458 (M+1).

### Example 139

### Methyl 4-(5-(4-(3-(2-fluorophenyl)ureido)phenyl)thiazol-2-yl)cyclohexane carboxylate

The compound of example 139 was prepared analogous to the compound of example 6 by reaction of the compound of example 132 with 1-isocyanato-2-fluorobenzene.

Yield: 62%; ¹H NMR (DMSO-d₆, 300 MHz): δ 9.22 (s, 1H), 8.57 (s, 1H), 8.17 (t, 1H), 7.958 (s, 1H), 7.57 (m, 4H), 7.27 (t, 1H), 7.17 (t, 1H), 7.05 (t, 1H), 3.61 (s, 3H), 2.99 (m, 1H), 2.42 (m, 1H), 2.16 (m, 2H), 2.03 (m, 2H), 1.58 (m, 4H); MS: m/z 454 (M+1); m/z 452 (M−1).

### Example 140

### 4-(5-(4-(3-(2-Fluorophenyl)ureido)phenyl)thiazol-2-yl)cyclohexanecarboxylic acid

The compound of example 140 was prepared analogous to the compound of example 7 by hydrolysis of the compound of example 139. Yield: 90%; ¹H NMR (DMSO-d₆, 300 MHz): δ 12.08 (s, 1H), 9.25 (s, 1H), 8.60 (s, 1H), 8.18 (t, 1H), 7.96 (s, 1H), 7.57 (m, 4H), 7.28 (t, 1H), 7.17 (t, 1H), 7.05 (t, 1H), 2.98 (m, 1H), 2.32 (m, 1H), 2.16 (m, 2H), 2.08 (m, 2H), 1.61 (m, 4H); MS: m/z 439 (M−1).

### Example 141

### Methyl 4-(5-(4-(3-cyclohexylureido)phenyl)thiazol-2-yl)cyclohexane carboxylate

The compound of example 141 was prepared analogous to the compound of example 6 by reaction of the compound of example 132 with 1-isocyanato cyclohexane. Yield: 80%; ¹H NMR (DMSO-d₆, 300 MHz): δ 8.45 (s, 1H), 7.90 (s, 1H), 7.48 (m, 4H), 6.12 (d, 1H), 3.61 (s, 3H), 3.48 (m, 1H), 2.98 (m, 1H), 2.40 (m, 1H), 2.15 (m, 2H), 2.08 (m, 2H), 1.82 (m, 2H), 1.65 (m, 2H), 1.57 (m, 4H), 1.36 (m, 2H), 1.33 (m, 4H); MS: m/z 442 (M+1); m/z 440 (M−1).

### Example 142

### 4-(5-(4-(3-Cyclohexylureido)phenyl)thiazol-2-yl)cyclohexanecarboxylic acid

The compound of example 142 was prepared analogous to the compound of example 7 by hydrolysis of the compound of example 141. Yield: 70%; ¹H NMR (DMSO-d₆, 300 MHz): δ 12.00 (s, 1H), 8.50 (s, 1H), 7.90 (s, 1H), 7.48 (m, 4H), 6.16 (d, 1H), 3.48 (m, 1H), 2.98 (m, 1H), 2.27 (m, 1H), 2.07 (m, 2H), 2.00 (m, 2H), 1.78 (m, 2H), 1.67 (m, 2H), 1.56 (m, 5H), 1.25 (m, 1H), 1.22 (m, 4H); MS: m/z 428 (M+1).

### Example 143

### Methyl 4-(5-(4-(3-(3-chlorophenyl)ureido)phenyl)thiazol-2-yl)cyclohexane carboxylate

The compound of example 143 was prepared analogous to the compound of example 6 by reaction of the compound of example 132 with 3-chloro-1-isocyanato benzene.

Yield: 45%; ¹H NMR (DMSO-d₆, 300 MHz): δ 8.35 (s, 1H), 8.32 (s, 1H), 7.76 (s, 1H), 7.56 (s, 1H), 7.50 (d, 2H), 7.41 (d, 2H), 7.30 (s, 1H), 7.20 (t, 1H), 6.96 (d, 1H), 3.72 (s, 3H), 3.04 (m, 1H), 2.29 (m, 2H), 2.14 (m, 2H), 1.68 (m, 4H), 1.26 (m, 1H); MS: m/z 470 (M+1); m/z 468 (M−1).

### Example 144

### 4-(5-(4-(3-(3-Chlorophenyl)ureido)phenyl)thiazol-2-yl)cyclohexane carboxylic acid

The compound of example 144 was prepared analogous to the compound of example 7 by hydrolysis of the compound of example 143. Yield: 43%; ¹H NMR (DMSO-d₆, 300 MHz): δ 9.06 (s, 1H), 9.04 (s, 1H), 7.96 (s, 1H), 7.71 (s, 1H), 7.57 (d, 4H), 7.31 (m, 2H), 7.04 (m, 1H), 2.99 (m, 1H), 2.28 (m, 1H), 2.16 (m, 2H), 2.03 (m, 2H), 1.57 (m, 4H); MS: m/z 456 (M+1); m/z 454 (M−1).

### Example 145

### Methyl 4-(5-(4-(3-(4-chlorophenyl)ureido)phenyl)thiazol-2-yl)cyclohexane carboxylate

The compound of example 145 was prepared analogous to the compound of example 6 by reaction of the compound of example 132 with 4-chloro-1-isocyanato benzene.

Yield: 64%; ¹H NMR (DMSO-d₆, 300 MHz): δ 8.19 (s, 1H), 8.16 (s, 1H), 7.72 (s, 1H), 7.50 (s, 1H), 7.46 (d, 2H), 7.40 (d, 2H), 7.28 (s, 1H), 7.23 (d, 2H), 3.67 (s, 3H), 2.96 (m, 1H), 2.37 (m, 1H), 2.27 (m, 2H), 2.12 (m, 2H), 1.67 (m, 4H); MS: m/z 470 (M+1); m/z 468 (M−1).

### Example 146

### 4-(5-(4-(3-(4-Chlorophenyl)ureido)phenyl)thiazol-2-yl)cyclohexane carboxylic acid

The compound of example 146 was prepared analogous to the compound of example 7 by hydrolysis of the compound of example 145. Yield: 90%; ¹H NMR (DMSO-d₆, 300 MHz): δ 9.68 (s, 1H), 7.66 (s, 1H), 7.96 (s, 1H), 7.53 (m, 5H), 7.48 (s, 1H), 7.34 (s, 1H), 7.31 (s, 1H), 2.99 (m, 1H), 2.29 (m, 1H), 2.16 (m, 2H), 2.03 (m, 2H), 1.57 (m, 4H); MS: m/z 456 (M+1); m/z 454 (M−1).

### Example 147

### Methyl 4-(5-(4-(3-(2-chloro-4-(trifluoromethyl)phenyl)ureido)phenyl)thiazol-2-yl)cyclohexanecarboxylate

The compound of example 147 was prepared analogous to the compound of example 6 by reaction of the compound of example 132 with 2-chloro-1-isocyanato-4-(trifluoromethyl)benzene. Yield: 59%; ¹H NMR (DMSO-d₆, 300 MHz): δ 9.29 (s, 1H), 8.45 (d, 1H), 8.25 (s, 1H), 7.76 (s, 1H), 7.69 (s, 1H), 7.66 (d, 2H), 7.95 (t, 3H), 3.64 (s, 3H), 3.04 (m, 1H), 2.36 (m, 1H), 2.27 (m, 2H), 2.17 (m, 2H), 1.65 (m, 4H); MS: m/z 538 (M+1); m/z 536 (M−1).

### Example 148

### 4-(5-(4-(3-(2-Chloro-4-(trifluoromethyl)phenyl)ureido)phenyl)thiazol-2-yl)cyclohexanecarboxylic acid

The compound of example 148 was prepared analogous to the compound of example 7 by hydrolysis of the compound of example 147. Yield: 83%; ¹H NMR (DMSO-d₆, 300 MHz): δ 12.12 (s, 1H), 9.77 (s, 1H), 8.66 (s, 1H), 8.49 (d, 1H), 7.98 (s, 1H), 7.88 (s, 1H), 7.71 (d, 1H), 7.60 (m, 4H), 3.00 (m, 1H), 2.28 (m, 1H), 2.16 (m, 2H), 2.03 (m, 2H), 1.61 (m, 4H); MS: m/z 524 (M+1).

### Example 149

### Methyl 4-(5-(4-(3-(2-chloro-5-methylphenyl)ureido)phenyl)thiazol-2-yl)cyclo hexanecarboxylate

The compound of example 149 was prepared analogous to the compound of example 6 by reaction of the compound of example 132 with 2-chloro-1-isocyanato-5-methyl benzene. Yield: 71%; ¹H NMR (DMSO-d₆, 300 MHz): δ 9.14 (s, 1H), 8.08 (s, 1H), 7.92 (s, 1H), 7.79 (s, 1H), 7.54 (d, 2H), 7.41 (d, 2H), 7.18 (d, 1H), 6.75 (d, 1H), 3.65 (s, 3H), 3.12 (m, 1H), 2.85 (m, 1H), 2.66 (m, 2H), 2.29 (s, 3H), 2.14 (m, 2H), 1.61 (m, 4H); MS: m/z 484 (M+1); m/z 482 (M−1).

### Example 150

### 4-(5-(4-(3-(2-Chloro-5-methylphenyl)ureido)phenyl)thiazol-2-yl)cyclohexane carboxylic acid

The compound of example 150 was prepared analogous to the compound of example 7 by hydrolysis of the compound of example 149. Yield: 63%; ¹H NMR (DMSO-d₆, 300 MHz): δ 9.72 (s, 1H), 8.34 (s, 1H), 8.00 (s, 1H), 7.98 (s, 1H), 7.58 (m, 4H), 7.34 (d, 1H), 6.88 (dd, 1H), 2.99 (m, 1H), 2.29 (bs, 4H), 2.21 (m, 2H), 2.13 (m, 2H), 1.50 (m, 4H); MS: m/z 470 (M+1); m/z 468 (M−1).

### Example 151

### Methyl 4-(5-(4-(3-(3-chloro-2-fluorophenyl)ureido)phenyl)thiazol-2-yl)cyclo hexanecarboxylate

The compound of example 151 was prepared analogous to the compound of example 6 by reaction of the compound of example 132 with 3-chloro-1-isocyanato-2-fluoro benzene. Yield: 63%; ¹H NMR (DMSO-d₆, 300 MHz): δ 9.27 (s, 1H), 8.74 (s, 1H), 8.12 (m, 1H), 7.96 (s, 1H), 7.58 (m, 4H), 7.19 (d, 2H), 3.61 (s, 3H), 3.01 (m, 1H), 2.40 (m, 1H), 2.16 (m, 2H), 2.03 (m, 2H), 1.58 (m, 4H); MS: m/z 488 (M+1).

### Example 152

### 4-(5-(4-(3-(3-Chloro-2-fluorophenyl)ureido)phenyl)thiazol-2-yl)cyclohexane carboxylic acid

The compound of example 152 was prepared analogous to the compound of example 7 by hydrolysis of the compound of example 151. Yield: 80%; ¹H NMR (DMSO-d₆, 300 MHz): δ 9.58 (s, 1H), 8.91 (s, 1H), 8.14 (m, 1H), 7.98 (s, 1H), 7.58 (m, 4H), 7.18 (d, 2H), 2.97 (m, 1H), 2.28 (m, 1H), 2.16 (m, 2H), 2.03 (m, 2H), 1.57 (m, 4H); MS: m/z 474.1 (M+1); m/z 472.1 (M−1).

### Example 153

### Methyl 4-(5-(4-(3-(4-methoxy-2-methylphenyl)ureido)phenyl)thiazol-2-yl)cyclohexanecarboxylate

The compound of example 153 was prepared analogous to the compound of example 6 by reaction of the compound of example 132 with 1-isocyanato-4-methoxy-2-methyl benzene. Yield: 66%; ¹H NMR (DMSO-d₆, 300 MHz): δ 8.99 (s, 1H), 7.94 (s, 1H), 7.82 (s, 1H), 7.54 (s, 1H), 7.51 (s, 4H), 6.79 (m, 2H), 3.72 (s, 3H), 3.61 (m, 3H), 3.00 (m, 1H), 2.40 (m, 1H), 2.15 (m, 2H), 2.00 (m, 2H), 1.55 (m, 4H); MS: m/z 480 (M+1); m/z 478 (M−1).

### Example 154

### 4-(5-(4-(3-(4-Methoxy-2-methylphenyl)ureido)phenyl)thiazol-2-yl)cyclo hexanecarboxylic acid

The compound of example 154 was prepared analogous to the compound of example 7 by hydrolysis of the compound of example 153. Yield: 42%; ¹H NMR (DMSO-d₆, 300 MHz): δ 9.12 (s, 1H), 7.94 (s, 1H), 7.91 (s, 1H), 7.55 (s, 1H), 7.52 (s, 4H), 6.78 (s, 1H), 6.75 (d, 1H), 3.72 (s, 3H), 2.96 (s, 1H), 2.28 (m, 1H), 2.22 (s, 3H), 2.15 (m, 2H), 2.03 (m, 2H), 1.57 (m, 4H); MS: m/z 466.2 (M+1); m/z 474.1 (M−1).

### Example 155

### Methyl 4-(5-(4-(3-benzo[d][1,3]dioxol-5-ylureido)phenyl)thiazol-2-yl)cyclo hexanecarboxylate

The compound of example 155 was prepared analogous to the compound of example 6 by reaction of the compound of example 132 with 5-isocyanato-benzo[1,3]dioxole.

Yield: 66%; ¹H NMR (DMSO-d₆, 300 MHz): δ 8.76 (s, 1H), 8.59 (s, 1H), 7.94 (s, 1H), 7.52 (m, 4H), 7.20 (s, 1H), 6.82 (m, 2H), 5.97 (s, 2H), 3.62 (s, 3H), 3.00 (m, 1H), 2.50 (m, 1H), 2.20 (m, 2H), 2.00 (m, 2H), 1.55 (m, 4H); MS: m/z 480 (M+1); m/z 478 (M−1).

### Example 156

### 4-(5-(4-(3-Benzo[d][1,3]-dioxol-5-ylureido)phenyl)thiazol-2-yl)cyclohexane carboxylic acid

The compound of example 156 was prepared analogous to the compound of example 7 by hydrolysis of the compound of example 155. Yield: 83%; ¹H NMR (DMSO-d₆, 300 MHz): δ 9.32 (s, 1H), 9.15 (s, 1H), 7.96 (s, 1H), 7.55 (m, 4H), 7.22 (d, 1H), 6.84 (d, 2H), 6.78 (dd, 1H), 5.97 (s, 2H), 2.99 (m, 1H), 2.28 (m, 1H), 2.16 (m, 2H), 2.12 (m, 2H), 1.57 (m, 4H); MS: m/z 466 (M+1); m/z 463 (M−1).

### Example 157

### Methyl 4-(5-(4-(3-(2-chloro-6-(trifluoromethyl)phenyl)ureido)phenyl)thiazol-2-yl)cyclohexanecarboxylate

The compound of example 157 was prepared analogous to the compound of example 6 by reaction of the compound of example 132 with 2-chloro-1-isocyanato-6-(trifluoromethyl)benzene. Yield: 59%; ¹H NMR (DMSO-d₆, 300 MHz): δ 9.12 (s, 1H), 8.22 (s, 1H), 7.95 (s, 1H), 7.91 (d, 1H), 7.78 (d, 1H), 7.58 (m, 5H), 3.61 (s, 3H), 2.97 (m, 1H), 2.38 (m, 1H), 2.16 (m, 2H), 2.03 (m, 2H), 1.58 (m, 4H); MS: m/z 538 (M+1); m/z 536 (M−1)

### Example 158

### 4-(5-(4-(3-(2-Chloro-6-(trifluoromethyl)phenyl)ureido)phenyl)thiazol-2-yl)cyclohexanecarboxylic acid

The compound of example 158 was prepared analogous to the compound of example 7 by hydrolysis of the compound of example 157. Yield: 77%; ¹H NMR (DMSO-d₆, 300 MHz): δ 12.15 (s, 1H), 9.16 (s, 1H), 8.24 (s, 1H), 7.95 (s, 1H), 7.91 (d, 1H), 7.78 (d, 1H), 7.58 (m, 5H), 2.98 (m, 1H), 2.28 (m, 1H), 2.15 (m, 2H), 2.03 (m, 2H), 1.57 (m, 4H); MS: m/z 524 (M+1); m/z 522 (M−1)

### Example 159

### Methyl 4-(5-(4-(3-(4-chloro-2-(trifluoromethyl)phenyl)ureido)phenyl)thiazol-2-yl)cyclohexanecarboxylate

The compound of example 159 was prepared analogous to the compound of example 6 by reaction of the compound of example 132 with 4-chloro-1-isocyanato-2-(trifluoromethyl)benzene. Yield: 59%; ¹H NMR (DMSO-d₆, 300 MHz): δ 9.56 (s, 1H), 8.19 (s, 1H), 8.02 (d, 1H), 7.99 (s, 1H), 7.75 (s, 1H), 7.66 (d, 1H), 7.55 (m, 4H), 3.61 (s, 3H), 2.99 (m, 1H), 2.38 (m, 1H), 2.16 (m, 2H), 2.03 (m, 2H), 1.63 (m, 4H); MS: m/z 538 (M+1); m/z 536 (M−1).

### Example 160

### 4-(5-(4-(3-(4-Chloro-2-(trifluoromethyl)phenyl)ureido)phenyl)thiazol-2-yl)cyclohexanecarboxylic acid

The compound of example 160 was prepared analogous to the compound of example 7 by hydrolysis of the compound of example 159. Yield: 77%; ¹H NMR (DMSO-d₆, 300 MHz): δ 12.10 (s, 1H), 9.74 (s, 1H), 8.29 (s, 1H), 8.01 (d, 1H), 7.98 (d, 1H), 7.74 (s, 1H), 7.71 (s, 1H), 7.58 (m, 4H), 2.95 (m, 1H), 2.30 (m, 1H), 2.15 (m, 2H), 2.03 (m, 2H), 1.57 (m, 4H); MS: m/z 522 (M+1); m/z 524 (M−1).

### Example 161

### Methyl 4-(5-(4-(3-(2-chloro-6-methylphenyl)ureido)phenyl)thiazol-2-yl)cyclo hexanecarboxylate

The compound of example 161 was prepared analogous to the compound of example 6 by reaction of the compound of example 132 with 2-chloro-1-isocyanato-6-methyl benzene. Yield: 41%; ¹H NMR (DMSO-d₆, 300 MHz): δ 9.07 (s, 1H), 8.01 (s, 1H), 7.94 (s, 1H), 7.52 (s, 4H), 7.23 (m, 1H), 7.19 (m, 2H), 3.61 (s, 3H), 2.90 (m, 1H), 2.41 (m, 1H), 2.26 (s, 3H), 2.13 (bs, 2H), 2.02 (bs, 2H), 1.54 (m, 4H); MS: m/z 484 (M+1); m/z 482 (M−1).

### Example 162

### 4-(5-(4-(3-(2-Chloro-6-methylphenyl)ureido)phenyl)thiazol-2-yl)cyclohexane carboxylic acid

The compound of example 162 was prepared analogous to the compound of example 7 by hydrolysis of the compound of example 161. Yield: 52%; ¹H NMR (DMSO-d₆, 300 MHz): δ 12.11 (s, 1H), 9.10 (s, 1H), 8.03 (s, 1H), 7.94 (s, 1H), 7.52 (s, 4H), 7.37 (d, 1H), 7.26 (m, 2H), 2.98 (m, 1H), 2.26 (bs, 4H), 2.15 (m, 2H), 2.03 (m, 2H), 1.61 (m, 4H); MS: m/z 470 (M+1); m/z 467 (M−1).

### Example 163

### Methyl 4-(5-(4-(3-(5-chloro-2-methylphenyl)ureido)phenyl)thiazol-2-yl)cyclo hexanecarboxylate

The compound of example 163 was prepared analogous to the compound of example 6 by reaction of the compound of example 132 with 5-chloro-1-isocyanato-2-methyl benzene. Yield: 41%; ¹H NMR (DMSO-d₆, 300 MHz): δ 9.28 (s, 1H), 8.06 (s, 1H), 8.05 (s, 1H), 7.93 (s, 1H), 7.56 (m, 4H), 7.20 (d, 1H), 6.99 (m, 1H), 6.75 (d, 1H), 3.61 (s, 3H), 2.99 (m, 1H), 2.43 (m, 1H), 2.25 (m, 3H), 2.17 (m, 2H), 2.06 (m, 2H), 1.59 (m, 4H); MS: m/z 484 (M+1); m/z 482 (M−1).

### Example 164

### 4-(5-(4-(3-(5-Chloro-2-methylphenyl)ureido)phenyl)thiazol-2-yl(cyclohexane carboxylic acid

The compound of example 164 was prepared analogous to the compound of example 7 by hydrolysis of the compound of example 163. Yield: 82%; ¹H NMR (DMSO-d₆, 300 MHz): δ 12.09 (s, 1H), 9.54 (s, 1H), 8.22 (s, 1H), 8.06 (s, 1H), 7.96 (s, 1H), 7.57 (m, 4H), 7.21 (d, 1H), 6.99 (dd, 1H), 2.99 (m, 1H), 2.26 (bs, 4H), 2.16 (m, 2H), 2.03 (m, 2H), 1.57 (m, 4H); MS: m/z 470 (M+1); m/z 468 (M−1).

### Example 165

### Methyl 4-(5-(4-(3-(2-(trifluoromethyl)phenyl)ureido)phenyl)thiazol-2-yl)cyclo hexanecarboxylate

The compound of example 165 was prepared analogous to the compound of example 6 by reaction of the compound of example 132 with 1-isocyanato-2-(trifluoromethyl)benzene.

Yield: 47%; ¹H NMR (DMSO-d₆, 300 MHz): δ 9.53 (s, 1H), 8.12 (s, 1H), 7.97 (s, 1H), 7.93 (s, 1H), 7.71 (m, 2H), 7.58 (m, 4H), 7.32 (t, 1H), 3.61 (s, 3H), 2.97 (m, 1H), 2.41 (m, 1H), 2.16 (m, 2H), 2.03 (m, 2H), 1.58 (m, 4H); MS: m/z 504 (M+1); MS: m/z 402 (M−1).

### Example 166

### 4-(5-(4-(3-(2-(Trifluoromethyl)phenyl)ureido)phenyl)thiazol-2-yl)cyclohexane carboxylic acid

The compound of example 166 was prepared analogous to the compound of example 7 by hydrolysis of the compound of example 165. Yield: 64%; ¹H NMR (DMSO-d₆, 300 MHz): δ 9.64 (s, 1H), 8.18 (s, 1H), 7.97 (s, 1H), 7.95 (d, 1H), 7.70 (m, 2H), 7.57 (m, 4H), 7.32 (t, 1H), 2.96 (m, 1H), 2.28 (m, 1H), 2.15 (m, 2H), 2.08 (m, 2H), 1.56 (m, 4H); MS: m/z 490 (M+1); MS: m/z 488 (M−1).

### Example 167

### Methyl 4-(5-(4-(3-(2-(trifluoromethoxy)phenyl)ureido)phenyl)thiazol-2-yl)cyclohexanecarboxylate

The compound of example 167 was prepared analogous to the compound of example 6 by reaction of the compound of example 132 with 1-isocyanato-2-(trifluoromethoxy)benzene.

Yield: 31%; ¹H NMR (DMSO-d₆, 300 MHz): δ 9.44 (s, 1H), 8.51 (s, 1H), 8.23 (d, 1H), 7.97 (s, 1H), 7.58 (m, 4H), 7.40 (m, 2H), 7.13 (t, 1H), 3.61 (s, 3H), 2.98 (m, 1H), 2.42 (m, 1H), 2.16 (m, 2H), 2.03 (m, 2H), 1.59 (m, 4H); MS: m/z 520 (M+1); m/z 518 (M−1).

### Example 168

### 4-(5-(4-(3-(2-(Trifluoromethoxy)phenyl)ureido)phenyl)thiazol-2-yl)cyclo hexanecarboxylic acid

The compound of example 168 was prepared analogous to the compound of example 7 by hydrolysis of the compound of example 167. Yield: 52%; ¹H NMR (DMSO-d₆, 300 MHz): δ 9.64 (s, 1H), 8.60 (s, 1H), 8.27 (d, 1H), 7.98 (s, 1H), 7.59 (m, 4H), 7.39 (m, 2H), 7.13 (t, 1H), 2.97 (m, 1H), 2.28 (m, 1H), 2.16 (m, 2H), 2.03 (m, 2H), 1.57 (m, 4H); MS: m/z 506 (M+1); m/z 504 (M−1).

### Example 169

### Methyl 4-(5-(4-(3-(4-phenoxyphenyl)ureido)phenyl)thiazol-2-yl)cyclohexane carboxylate

The compound of example 169 was prepared analogous to the compound of example 6 by reaction of the compound of example 132 with 1-isocyanato-4-phenoxy benzene.

Yield: 47%; ¹H NMR (DMSO-d₆, 300 MHz): δ 8.84 (s, 1H), 8.74 (s, 1H), 7.95 (s, 1H), 7.53 (m, 4H), 7.49 (s, 1H), 7.46 (s, 1H), 7.39 (t, 2H), 3.12 (t, 1H), 7.01 (m, 4H), 3.61 (s, 3H), 2.97 (m, 1H), 2.42 (m, 1H), 2.13 (m, 2H), 2.03 (m, 2H), 1.55 (m, 4H); MS: m/z 528 (M+1); m/z 526 (M−1).

### Example 170

### 4-(5-(4-(3-(4-Phenoxyphenyl)ureido)phenyl)thiazol-2-yl)cyclohexane carboxylic acid

The compound of example 170 was prepared analogous to the compound of example 7 by hydrolysis of the compound of example 169. Yield: 40%; ¹H NMR (DMSO-d₆, 300 MHz): δ 12.12 (s, 1H), 8.85 (s, 1H), 8.75 (s, 1H), 7.95 (s, 1H), 7.53 (bs, 4H), 7.49 (s, 1H), 7.47 (s, 1H), 7.39 (t, 2H), 3.11 (t, 1H), 7.00 (m, 4H), 2.98 (m, 1H), 2.27 (m, 1H), 2.12 (m, 2H), 2.03 (m, 2H), 1.55 (m, 4H); MS: m/z 514 (M+1); m/z 512 (M−1).

### Example 171

### Methyl 4-(5-(4-(3-(4-chloro-2-fluorophenyl)ureido)phenyl)thiazol-2-yl)cyclo hexanecarboxylate

The compound of example 171 was prepared analogous to the compound of example 6 by reaction of the compound of example 132 with 4-chloro-2-fluoro-1-isocyanato benzene. Yield: 81%; ¹H NMR (DMSO-d₆, 300 MHz): δ 9.21 (s, 1H), 8.66 (s, 1H), 8.18 (t, 1H), 7.94 (s, 1H), 7.55 (m, 5H), 7.23 (d, 1H), 3.59 (s, 3H), 2.95 (m, 1H), 2.38 (m, 1H), 2.10 (m, 2H), 2.00 (m, 2H), 1.56 (m, 4H); MS: m/z 488 (M+1); m/z 486 (M−1).

### Example 172

### 4-(5-(4-(3-(4-Chloro-2-fluorophenyl)ureido)phenyl)thiazol-2-yl)cyclohexane carboxylic acid

The compound of example 172 was prepared analogous to the compound of example 7 by hydrolysis of the compound of example 171. Yield: 87%; ¹H NMR (DMSO-d₆, 300 MHz): δ 9.56 (s, 1H), 8.83 (s, 1H), 8.20 (t, 1H), 7.98 (s, 1H), 7.57 (m, 3H), 7.45 (d, 2H), 7.25 (d, 1H), 2.97 (m, 1H), 2.28 (m, 1H), 2.12 (m, 2H), 2.03 (m, 2H), 1.57 (m, 4H); MS: m/z 474 (M+1); m/z 472 (M−1).

### Example 173

### Methyl 4-(5-(4-(3-(2-fluoro-5-methyl phenyl)ureido)phenyl)thiazol-2-yl)cyclo hexanecarboxylate

The compound of example 173 was prepared analogous to the compound of example 6 by reaction of the compound of example 132 with 1-isocyanato-2-fluoro-5-methyl benzene. Yield: 76%; ¹H NMR (DMSO-d₆, 300 MHz): δ 9.19 (s, 1H), 8.49 (s, 1H), 7.97 (s, 1H), 7.94 (s, 1H), 7.54 (m, 4H), 7.12 (m, 1H), 6.78 (m, 1H), 3.59 (s, 3H), 2.95 (m, 1H), 2.38 (m, 1H), 2.25 (s, 3H), 2.10 (m, 2H), 2.00 (m, 2H), 1.60 (m, 4H); MS: m/z 468 (M+1); m/z 466 (M−1).

### Example 174

### 4-(5-(4-(3-(2-Fluoro-5-methyl phenyl)ureido)phenyl)thiazol-2-yl)cyclohexane carboxylic acid

The compound of example 174 was prepared analogous to the compound of example 7 by hydrolysis of the compound of example 173. Yield: 50%; ¹H NMR (DMSO-d₆, 300 MHz): δ 9.41 (s, 1H), 8.60 (s, 1H), 7.98 (s, 1H), 7.97 (s, 1H), 7.57 (m, 4H), 7.14 (m, 1H), 6.81 (m, 1H), 2.99 (m, 1H), 2.51 (m, 1H), 2.27 (s, 3H), 2.17 (m, 2H), 2.03 (m, 2H), 1.51 (m, 4H); MS: m/z 454 (M+1); m/z 452 (M−1).

### Example 175

### Methyl 4-(5-(4-(3-(2-fluoro-6-(trifluoromethyl)phenyl)ureido)phenyl)thiazol-2-yl)cyclohexanecarboxylate

The compound of example 175 was prepared analogous to the compound of example 6 by reaction of the compound of example 132 with 1-isocyanato-2-fluoro-6-(trifluoromethyl)benzene. Yield: 68%; ¹H NMR (DMSO-d₆, 300 MHz): δ 9.16 (s, 1H), 8.08 (s, 1H), 7.93 (s, 1H), 7.66 (m, 2H), 7.55 (m, 5H), 3.59 (s, 3H), 2.94 (m, 1H), 2.40 (m, 1H), 2.10 (m, 2H), 2.00 (m, 2H), 1.56 (m, 4H); MS: m/z 522 (M+1); m/z 520 (M−1).

### Example 176

### 4-(5-(4-(3-(2-Fluoro-6-(trifluoromethyl)phenyl)ureido)phenyl)thiazol-2-yl)cyclohexanecarboxylic acid

The compound of example 176 was prepared analogous to the compound of example 7 by hydrolysis of the compound of example 175. Yield: 80%; ¹H NMR (DMSO-d₆, 300 MHz): δ 12.15 (s, 1H), 9.40 (s, 1H), 8.24 (s, 1H), 7.95 (s, 1H), 7.68 (m, 2H), 7.57 (m, 5H), 2.99 (m, 1H), 2.32 (m, 1H), 2.15 (m, 2H), 2.02 (m, 2H), 1.63 (m, 4H); MS: m/z 508 (M+1); m/z 506 (M−1).

### Example 177

### Methyl 4-(5-(4-(3-(3-fluorophenyl)ureido)phenyl)thiazol-2-yl)cyclohexane carboxylate

The compound of example 177 was prepared analogous to the compound of example 6 by reaction of the compound of example 132 with 1-isocyanato-3-fluoro benzene.

Yield: 96%; ¹H NMR (DMSO-d₆, 300 MHz): δ 8.92 (s, 1H), 8.88 (s, 1H), 7.93 (s, 1H), 7.54 (m, 5H), 7.32 (m, 1H), 7.12 (d, 1H), 6.79 (t, 1H), 3.59 (s, 3H), 2.95 (m, 1H), 2.38 (m, 1H), 2.10 (m, 2H), 2.00 (m, 2H), 1.60 (m, 4H); MS: m/z 454 (M+1); m/z 452 (M−1).

### Example 178

### 4-(5-(4-(3-(3-Fluorophenyl)ureido)phenyl)thiazol-2-yl)cyclohexanecarboxylic acid

The compound of example 178 was prepared analogous to the compound of example 7 by hydrolysis of the compound of example 177. Yield: 87%; ¹H NMR (DMSO-d₆, 300 MHz): δ 9.35 (s, 1H), 9.27 (s, 1H), 7.98 (s, 1H), 7.57 (m, 5H), 7.32 (m, 1H), 7.14 (d, 1H), 6.79 (t, 1H), 3.01 (m, 1H), 2.32 (m, 1H), 2.13 (m, 2H), 2.03 (m, 2H), 1.62 (m, 4H); MS: m/z 438 (M−1).

### Example 179

### Methyl 4-(5-(4-(3-(3,4-difluorophenyl)ureido)phenyl)thiazol-2-yl)cyclohexane carboxylate

The compound of example 179 was prepared analogous to the compound of example 6 by reaction of the compound of example 132 with 1-isocyanato-3,4-difluoro benzene.

Yield: 67%; ¹H NMR (DMSO-d₆, 300 MHz): δ 8.90 (bs, 2H), 7.93 (s, 1H), 7.64 (s, 1H), 7.49 (m, 4H), 7.34 (m, 1H), 7.12 (m, 1H), 3.59 (s, 3H), 2.95 (m, 1H), 2.48 (m, 1H), 2.10 (m, 2H), 1.99 (m, 2H), 1.52 (m, 4H); MS: m/z 472 (M+1); m/z 470 (M−1).

### Example 180

### 4-(5-(4-(3-(3,4-Difluorophenyl)ureido)phenyl)thiazol-2-yl)cyclohexane carboxylic acid

The compound of example 180 was prepared analogous to the compound of example 7 by hydrolysis of the compound of example 179. Yield: 52%; ¹H NMR (DMSO-d₆, 300 MHz): δ 9.17 (bs, 1H), 9.11 (bs, 1H), 7.96 (s, 1H), 7.66 (m, 1H), 7.53 (m, 4H), 7.37 (m, 1H), 7.14 (m, 1H), 2.96 (m, 1H), 2.28 (m, 1H), 2.12 (m, 2H), 2.03 (m, 2H), 1.56 (m, 4H); MS: m/z 458 (M+1).

### Example 181

### Methyl 4-(5-(4-(3-(3,5-difluorophenyl)ureido)phenyl)thiazol-2-yl)cyclohexane carboxylate

The compound of example 181 was prepared analogous to the compound of example 6 by reaction of the compound of example 132 with 1-isocyanato-3,5-difluoro benzene.

Yield: 75%; ¹H NMR (DMSO-d₆, 300 MHz): δ 9.10 (bs, 1H), 8.99 (bs, 1H), 7.94 (s, 1H), 7.55 (m, 4H), 7.18 (d, 1H), 7.16 (d, 1H), 6.81 (m, 1H), 3.59 (s, 3H), 2.95 (m, 1H), 2.38 (m, 1H), 2.10 (m, 2H), 2.00 (m, 2H), 1.56 (m, 4H); MS: m/z 472 (M+1); m/z 470 (M−1).

### Example 182

### 4-(5-(4-(3-(3,5-Difluorophenyl)ureido)phenyl)thiazol-2-yl)cyclohexane carboxylic acid

The compound of example 182 was prepared analogous to the compound of example 7 by hydrolysis of the compound of example 181. Yield: 61%; ¹H NMR (DMSO-d₆, 300 MHz): δ 9.39 (bs, 1H), 9.21 (bs, 1H), 7.97 (s, 1H), 7.57 (m, 4H), 7.20 (d, 1H), 7.18 (d, 1H), 6.83 (m, 1H), 2.96 (m, 1H), 2.28 (m, 1H), 2.12 (m, 2H), 2.03 (m, 2H), 1.57 (m, 4H); MS: m/z 458 (M+1); m/z 456 (M−1).

### Example 183

### Methyl 4-(5-(4-(3-(2,6-difluorophenyl)ureido)phenyl)thiazol-2-yl)cyclohexane carboxylate

The compound of example 183 was prepared analogous to the compound of example 6 by reaction of the compound of example 132 with 1-isocyanato-2,6-difluoro benzene.

¹H NMR (DMSO-d₆, 300 MHz): δ 9.11 (s, 1H), 8.16 (s, 1H), 7.95 (s, 1H), 7.55 (m, 4H), 7.32 (m, 1H), 7.19 (t, 2H), 3.61 (s, 3H), 2.97 (m, 1H), 2.44 (m, 1H), 2.15 (m, 2H), 2.03 (m, 2H), 1.58 (m, 4H); MS: m/z 472 (M+1); m/z 470 (M−1).

### Example 184

### 4-(5-(4-(3-(2,6-Difluorophenyl)ureido)phenyl)thiazol-2-yl)cyclohexane carboxylic acid

The compound of example 184 was prepared analogous to the compound of example 7 by hydrolysis of the compound of example 183. ¹H NMR (DMSO-d₆, 300 MHz): δ 9.38 (s, 1H), 8.35 (s, 1H), 7.98 (s, 1H), 7.56 (m, 4H), 7.35 (m, 1H), 7.19 (t, 2H), 2.99 (m, 1H), 2.28 (m, 1H), 2.16 (m, 2H), 2.03 (m, 2H), 1.57 (m, 4H); MS: m/z 458.1 (M+1); m/z 456.1 (M−1).

### Example 185

### Methyl 4-(5-(4-(3-(2,3,4-trifluorophenyl)ureido)phenyl)thiazol-2-yl)cyclo hexanecarboxylate

The compound of example 185 was prepared analogous to the compound of example 6 by reaction of the compound of example 132 with 1-isocyanato-2,3,4-trifluoro benzene. ¹H NMR (DMSO-d₆, 300 MHz): δ 9.21 (s, 1H), 8.72 (s, 1H), 7.96 (s, 1H), 7.88 (m, 1H), 7.57 (m, 4H), 7.29 (m, 1H), 3.61 (s, 3H), 2.97 (m, 1H), 2.44 (m, 1H), 2.15 (m, 2H), 2.03 (m, 2H), 1.62 (m, 4H); MS: m/z 490 (M+1); m/z 488 (M−1).

### Example 186

### 4-(5-(4-(3-(2,3,4-Trifluorophenyl)ureido)phenyl)thiazol-2-yl)cyclohexane carboxylic acid

The compound of example 186 was prepared analogous to the compound of example 7 by hydrolysis of the compound of example 185. ¹H NMR (DMSO-d₆, 300 MHz): δ 12.13 (s, 1H), 9.31 (s, 1H), 8.77 (s, 1H), 7.96 (s, 1H), 7.91 (m, 1H), 7.57 (m, 4H), 7.32 (m, 1H), 2.96 (m, 1H), 2.36 (m, 1H), 2.16 (m, 2H), 2.03 (m, 2H), 1.62 (m, 4H); MS: m/z 476.1 (M+1); m/z 474.1 (M−1).

### Example 187

### Methyl 4-(5-(4-(3-(2-chlorophenyl)ureido)Phenyl)thiazol-2-yl)cyclohexane carboxylate

The compound of example 187 was prepared analogous to the compound of example 6 by reaction of the compound of example 132 with 2-chloro-1-isocyanato benzene.

Yield: 83%; ¹H NMR (DMSO-d₆, 300 MHz): δ 9.56 (s, 1H), 8.34 (s, 1H), 8.18 (dd, 1H), 7.96 (s, 1H), 7.58 (m, 4H), 7.48 (dd, 1H), 7.30 (m, 1H), 7.07 (m, 1H), 3.61 (s, 3H), 2.97 (m, 1H), 2.41 (m, 1H), 2.16 (m, 2H), 2.03 (m, 2H), 1.58 (m, 4H); MS: m/z 470.1 (M+1).

### Example 188

### 4-(5-(4-(3-(2-Chlorophenyl)ureido)phenyl)thiazol-2-yl)cyclohexane carboxylic acid

The compound of example 188 was prepared analogous to the compound of example 7 by hydrolysis of the compound of example 187. Yield: 75%; ¹H NMR (DMSO-d₆, 300 MHz): δ 12.00 (bs, 1H), 9.58 (s, 1H), 8.36 (s, 1H), 8.17 (dd, 1H), 7.96 (s, 1H), 7.58 (m, 4H), 7.48 (dd, 1H), 7.33 (m, 1H), 7.07 (m, 1H), 2.96 (m, 1H), 2.31 (m, 1H), 2.16 (m, 2H), 2.03 (m, 2H), 1.61 (m, 4H); MS: m/z 456.1 (M+1).

### Example 189

### Methyl 4-(5-(4-(3-(4-chloro-2-phenoxyphenyl)ureido)phenyl)thiazol-2-yl)cyclohexanecarboxylate

The compound of example 189 was prepared analogous to the compound of example 6 by reaction of the compound of example 132 with 4-chloro-1-isocyanato-2-phenoxy benzene. Yield: 76%; ¹H NMR (DMSO-d₆, 300 MHz): δ 9.50 (s, 1H), 8.69 (s, 1H), 8.39 (d, 1H), 7.95 (s, 1H), 7.56 (m, 4H), 7.44 (d, 2H), 7.19 (t, 1H), 7.10 (d, 2H), 7.01 (dd, 1H), 6.85 (d, 1H), 3.61 (s, 3H), 3.00 (m, 1H), 2.41 (m, 1H), 2.12 (m, 2H), 2.02 (m, 2H), 1.55 (m, 4H); MS: m/z 562.2 (M+1).

### Example 190

### 4-(5-(4-(3-(4-Chloro-2-phenoxyphenyl)ureido)phenyl)thiazol-2-yl)cyclohexane carboxylic acid

The compound of example 190 was prepared analogous to the compound of example 7 by hydrolysis of the compound of example 189. Yield: 96%; ¹H NMR (DMSO-d₆, 300 MHz): δ 12.00 (bs, 1H), 9.52 (s, 1H), 8.70 (s, 1H), 8.40 (d, 1H), 7.96 (s, 1H), 7.57 (m, 4H), 7.44 (d, 2H), 7.22 (t, 1H), 7.10 (d, 2H), 7.02 (dd, 1H), 6.85 (d, 1H), 2.98 (m, 1H), 2.27 (m, 1H), 2.15 (m, 2H), 2.03 (m, 2H), 1.56 (m, 4H); MS: m/z 548.2 (M+1).

### Example 191

### Methyl 4-(5-(4-(3-phenylureido)phenyl)thiazol-2-yl)cyclohexanecarboxylate

The compound of example 191 was prepared analogous to the compound of example 6 by reaction of the compound of example 132 with isocyanato benzene.

Yield: 71%; ¹H NMR (DMSO-d₆, 300 MHz): δ 8.83 (s, 1H), 8.64 (s, 1H), 7.95 (s, 1H), 7.52 (m, 4H), 7.47 (d, 2H), 7.31 (t, 2H), 7.00 (t, 1H), 3.61 (s, 3H), 2.89 (m, 1H), 2.40 (m, 1H), 2.15 (m, 2H), 2.03 (m, 2H), 1.58 (m, 4H); MS: m/z 436.2 (M+1).

### Example 192

### 4-(5-(4-(3-Phenylureido)phenyl)thiazol-2-yl)cyclohexanecarboxylic acid

The compound of example 192 was prepared analogous to the compound of example 7 by hydrolysis of the compound of example 191. Yield: 85%; ¹H NMR (DMSO-d₆, 300 MHz): δ 11.60 (s, 1H), 11.38 (s, 1H), 7.91 (s, 1H), 7.67 (m, 4H), 7.51 (d, 2H), 7.23 (m, 2H), 6.89 (m, 1H), 2.92 (m, 1H), 2.13 (m, 5H), 1.51 (m, 4H); MS: m/z 422.2 (M+1).

### Example 193

### Methyl 4-(5-(4-(4-tert-butylbenzamido)phenyl)thiazol-2-yl)cyclohexane carboxylate

The compound of example 193 was prepared analogous to the compound of example 14 by reaction of the compound of example 132 with 4-(t-butyl)benzoyl chloride. Yield: 73%; ¹H NMR (DMSO-d₆, 300 MHz): δ 10.31 (s, 1H), 8.01 (s, 1H), 7.91 (d, 2H), 7.87 (d, 2H), 7.62 (d, 2H), 7.56 (d, 2H), 3.61 (s, 3H), 2.98 (m, 1H), 2.40 (m, 1H), 2.16 (m, 2H), 2.03 (m, 2H), 1.59 (m, 4H), 1.32 (s, 9H); MS: m/z 477.2 (M+1).

### Example 194

### 4-(5-(4-(4-t-Butylbenzamido)phenyl)thiazol-2-yl)cyclohexanecarboxylic acid

The compound of example 194 was prepared analogous to the compound of example 15 by hydrolysis of the compound of example 193. Yield: 84%; ¹H NMR (DMSO-d₆, 300 MHz): δ 12.15 (bs, 1H), 10.30 (s, 1H), 8.00 (s, 1H), 7.91 (d, 2H), 7.86 (d, 2H), 7.62 (d, 2H), 7.56 (d, 2H), 2.99 (m, 1H), 2.31 (m, 1H), 2.16 (m, 2H), 2.03 (m, 2H), 1.61 (m, 4H), 1.32 (s, 9H); MS: m/z 463.2 (M+1).

### Example 195

### Methyl 4-(5-(4-(2-chlorobenzamido)phenyl)thiazol-2-yl)cyclohexane carboxylate

The compound of example 195 was prepared analogous to the compound of example 14 by reaction of the compound of example 132 with 2-chloro benzoyl chloride. Yield: 69%; ¹H NMR (DMSO-d₆, 300 MHz): δ 10.64 (s, 1H), 8.88 (d, 1H), 8.46 (t, 1H), 8.01 (s, 1H), 7.98 (t, 1H), 7.79 (d, 2H), 7.63 (d, 2H), 7.54 (m, 1H), 3.61 (s, 3H), 3.01 (m, 1H), 2.42 (m, 1H), 2.16 (m, 2H), 2.03 (m, 2H), 1.59 (m, 4H); MS: m/z 455.1 (M+1).

### Example 196

### 4-(5-(4-(2-Chlorobenzamido)phenyl)thiazol-2-yl)cyclohexanecarboxylic acid

The compound of example 196 was prepared analogous to the compound of example 15 by hydrolysis of the compound of example 195. Yield: 95%; ¹H NMR (DMSO-d₆, 300 MHz): δ 12.00 (bs, 1H), 10.64 (s, 1H), 8.00 (s, 1H), 7.79 (d, 2H), 7.63 (d, 2H), 7.59 (m, 2H), 7.52 (m, 2H), 2.96 (m, 1H), 2.26 (m, 1H), 2.16 (m, 2H), 2.03 (m, 2H), 1.57 (m, 4H); MS: m/z 441.1 (M+1).

### Example 197

### Methyl 4-(5-(4-(5-phenyloxazole-2-carboxamido)phenyl)thiazol-2-yl)cyclo hexane carboxylate

The compound of example 197 was prepared analogous to the compound of example 14 by reaction of the compound of example 132 with 5-phenyl-oxazole-2-carbonyl chloride. Yield: 31%; ¹H NMR (DMSO-d₆, 300 MHz): δ 11.00 (s, 1H), 8.08 (s, 2H), 7.93 (t, 4H), 7.66 (d, 2H), 7.59 (t, 2H), 7.49 (m, 1H), 3.61 (s, 3H), 2.99 (m, 1H), 2.43 (m, 1H), 2.17 (m, 2H), 2.03 (m, 2H), 1.59 (m, 4H); MS: m/z 488.2 (M+1).

### Example 198

### 4-(5-(4-(5-Phenyloxazole-2-carboxamido)phenyl)thiazol-2-yl)cyclohexane carboxylic acid

The compound of example 198 was prepared analogous to the compound of example 15 by hydrolysis of the compound of example 197. Yield: 94%; ¹H NMR (DMSO-d₆, 300 MHz): δ 12.09 (bs, 1H), 10.98 (s, 1H), 8.03 (s, 2H), 7.93 (t, 4H), 7.66 (d, 2H), 7.57 (t, 2H), 7.49 (m, 1H), 2.99 (m, 1H), 2.27 (m, 1H), 2.16 (m, 2H), 2.03 (m, 2H), 1.56 (m, 4H); MS: m/z 474.1 (M+1).

### Example 199

### Methyl 4-(5-(4-(3-(4-methoxyphenyl)thioureido)phenyl)thiazol-2-yl)cyclo hexanecarboxylate

The compound of example 199 was prepared analogous to the compound of example 6 by reaction of the compound of example 132 with 1-isothiocyanato-4-methoxy benzene. Yield: 83%; ¹H NMR (DMSO-d₆, 300 MHz): δ 9.75 (s, 1H), 9.70 (s, 1H), 8.00 (s, 1H), 7.55 (s, 4H), 7.35 (d, 2H), 6.93 (d, 2H), 3.75 (s, 3H), 3.61 (s, 3H), 2.98 (m, 1H), 2.42 (m, 1H), 2.16 (m, 2H), 2.03 (m, 2H), 1.58 (m, 4H); MS: m/z 482 (M+1); m/z 480 (M−1).

### Example 200

### Methyl 4-(5-(4-(3-(4-chlorophenyl)thioureido)phenyl)thiazol-2-yl)cyclo hexanecarboxylate

The compound of example 200 was prepared analogous to the compound of example 6 by reaction of the compound of example 132 with 1-chloro-4-isothiocyanato benzene.

Yield: 57%; ¹H NMR (DMSO-d₆, 300 MHz): δ 9.99 (s, 1H); 9.95 (s, 1H), 8.02 (s, 1H), 7.61 (s, 6H), 7.40 (s, 1H), 6.38 (s, 1H), 3.62 (s, 3H), 3.02 (m, 1H), 2.40 (m, 1H), 2.14 (m, 2H), 2.03 (m, 2H), 1.64 (m, 4H); MS: m/z 486 (M+1); 484 (M−1).

### Example 201

### Methyl 4-(5-(4-nitrophenyl)oxazol-2-yl)cyclohexanecarboxylate

To the compound of example 130 (0.150 g) in acetonitrile (8 mL), was added POCl₃ (0.108 mL), and the reaction mixture was refluxed for 5 h. The reaction mixture was cooled to room temperature, ice was added and aqueous NaHCO₃ solution was added to obtain neutral pH. The reaction mixture was extracted with ethyl acetate. The organic solvent was concentrated and the crude residue obtained was purified by crystallization in methanol to afford the title compound. Yield: 85 mg (54%); ¹H NMR (CDCl₃, 300 MHz): δ 8.30 (d, 2H), 7.78 (d, 2H), 7.45 (s, 1H), 3.27 (s, 3H), 2.90 (m, 1H), 2.42 (m, 1H), 2.32 (m, 2H), 2.20 (m, 2H), 1.76 (m, 4H); MS: m/z 331.1 (M+1).

### Example 202

### Methyl 4-(5-(4-aminophenyl)oxazol-2-yl)cyclohexane carboxylate

The compound of example 202 was prepared analogous to the compound of example 5 by reduction of the compound of example 201. Yield: 84%; ¹H NMR (DMSO-d₆, 300 MHz): δ 7.32 (d, 2H); 7.13 (s, 1H), 6.60 (d, 2H), 5.39 (s, 2H), 3.60 (s, 3H), 2.80 (m, 1H), 2.41 (m, 1H), 2.12 (m, 2H), 2.00 (m, 2H), 1.56 (m, 4H); MS: m/z 300.8 (M+1).

### Example 203

### Methyl 4-(5-(4-(3-(2-chlorophenyl)ureido)phenyl)oxazol-2-yl)cyclohexane carboxylate

The compound of example 203 was prepared analogous to the compound of example 6 by reaction of the compound of example 202 with 1-chloro-2-isocyanato benzene.

Yield: 57%; ¹H NMR (DMSO-d₆, 300 MHz): δ 9.58 (s, 1H), 8.34 (s, 1H), 8.18 (d, 1H), 7.62 (d, 2H), 7.56 (d, 2H), 7.48 (d, 1H), 7.41 (s, 1H), 7.33 (t, 1H), 7.07 (t, 1H), 3.61 (s, 3H), 2.84 (m, 1H), 2.40 (m, 1H), 2.15 (m, 2H), 2.02 (m, 2H), 1.59 (m, 4H); MS: m/z 452.2 (M+1).

### Example 204

### 4-(5-(4-(3-(2-Chlorophenyl)ureido)phenyl)oxazol-2-yl)cyclohexanecarboxylic acid

The compound of example 204 was prepared analogous to the compound of example 7 by hydrolysis of the compound of example 203. Yield: 73%; ¹H NMR (DMSO-d₆, 300 MHz): δ 9.58 (s, 1H), 8.34 (s, 1H), 8.18 (dd, 1H), 7.63 (d, 2H), 7.56 (d, 2H), 7.48 (dd, 1H), 7.40 (s, 1H), 7.31 (m, 1H), 7.04 (m, 1H), 2.84 (m, 1H), 2.30 (m, 1H), 2.15 (m, 2H), 2.01 (m, 2H), 1.58 (m, 4H); MS: m/z 438.2 (M−1).

### Example 205

### Methyl 4-(5-(4-(3-phenylureido)phenyl)oxazol-2-yl)cyclohexanecarboxylate

The compound of example 205 was prepared analogous to the compound of example 6 by reaction of the compound of example 202 with isocyanato benzene.

Yield: 81%; ¹H NMR (DMSO-d₆, 300 MHz): δ 8.84 (s, 1H), 8.69 (s, 1H), 7.60 (d, 2H), 7.55 (d, 2H), 7.47 (d, 2H), 7.39 (s, 1H), 7.31 (t, 2H), 7.00 (t, 1H), 3.61 (s, 3H), 2.84 (m, 1H), 2.39 (m, 1H), 2.15 (m, 2H), 2.02 (m, 2H), 1.59 (m, 4H); MS: m/z 420.2 (M+1).

### Example 206

### 4-(5-(4-(3-Phenylureido)phenyl)oxazol-2-yl)cyclohexanecarboxylic acid

The compound of example 206 was prepared analogous to the compound of example 7 by hydrolysis of the compound of example 205. Yield: 77%; ¹H NMR (DMSO-d₆; 300 MHz): δ 8.89 (s, 1H), 8.75 (s, 1H), 7.60 (d, 2H), 7.55 (d, 2H), 7.47 (d, 2H), 7.39 (s, 1H), 7.31 (t, 2H), 7.00 (t, 1H), 2.86 (m, 1H), 2.30 (m, 1H), 2.15 (m, 2H), 2.01 (m, 2H), 1.57 (m, 4H); MS: m/z 406.2 (M+1).

### Example 207

### Methyl 4-(5-(4-(3-(3-chlorophenyl)ureido)phenyl)oxazol-2-yl)cyclohexane carboxylate

The compound of example 207 was prepared analogous to the compound of example 6 by reaction of the compound of example 202 with 1-chloro-3-isocyanato benzene.

Yield: 86%; ¹H NMR (DMSO-d₆, 300 MHz): δ 8.93 (s, 1H), 8.92 (s, 1H), 7.72 (s, 1H), 7.61 (d, 2H), 7.55 (d, 2H), 7.40 (s, 1H), 7.33 (m, 2H), 7.04 (d, 1H), 3.61 (s, 3H), 2.86 (m, 1H), 2.40 (m, 1H), 2.15 (m, 2H), 2.02 (m, 2H), 1.59 (m, 4H); MS: m/z 454.1 (M+1).

### Example 208

### 4-(5-(4-(3-(3-Chlorophenyl)ureido)phenyl)oxazol-2-yl)cyclohexanecarboxylic acid

The compound of example 208 was prepared analogous to the compound of example 7 by hydrolysis of the compound of example 207. Yield: 92%; ¹H NMR (DMSO-d₆, 300 MHz): δ 9.26 (s, 1H), 9.23 (s, 1H), 7.71 (s, 1H), 7.61 (d, 2H), 7.55 (d, 2H), 7.40 (s, 1H), 7.33 (m, 2H), 7.0 m (d, 1H), 2.82 (m, 1H), 2.28 (m, 1H), 2.15 (m, 2H), 2.01 (m, 2H), 1.57 (m, 4H); MS: m/z 440.1 (M+1).

### Example 209

### Methyl 4-(5-(4-(3-(2-methoxyphenyl)ureido)phenyl)oxazol-2-yl)cyclohexane carboxylate

The compound of example 209 was prepared analogous to the compound of example 6 by reaction of the compound of example 202 with 1-isocyanato-2-methoxy benzene.

Yield: 40%; ¹H NMR (DMSO-d₆, 300 MHz): δ 9.48 (s, 1H), 8.26 (s, 1H), 8.14 (d, 1H), 7.60 (d, 2H), 7.54 (d, 2H), 7.39 (s, 1H), 7.04 (m, 3H), 3.88 (s, 3H), 3.61 (s, 3H), 2.84 (m, 1H), 2.40 (m, 1H), 2.15 (m, 2H), 2.02 (m, 2H), 1.59 (m, 4H); MS: m/z 448.2 (M−1).

### Example 210

### 4-(5-(4-(3-(2-Methoxyphenyl)ureido)phenyl)oxazol-2-yl)cyclohexane carboxylic acid

The compound of example 210 was prepared analogous to the compound of example 7 by hydrolysis of the compound of example 209. Yield: 76%; ¹H NMR (DMSO-d₆; 300 MHz): δ 12.12 (s, 1H), 9.48 (s, 1H), 8.26 (s, 1H), 8.13 (d, 1H), 7.57 (d, 4H), 7.38 (s, 1H), 7.01 (m, 3H), 3.88 (s, 3H), 2.85 (m, 1H), 2.26 (m, 1H), 2.11 (m, 2H), 2.01 (m, 2H), 1.57 (m, 4H); MS: m/z 436.2 (M+1).

### Example 211

### Methyl 4-(5-(4-(2-chlorobenzamido)phenyl)oxazol-2-yl)cyclohexane carboxylate

The compound of example 211 was prepared analogous to the compound of example 14 by reaction of the compound of example 202 with 2-chloro benzoyl chloride. Yield: 77%; ¹H NMR (DMSO-d₆, 300 MHz): δ 10.60 (s, 1H), 7.92 (m, 1H), 7.82 (d, 2H), 7.67 (d, 2H), 7.62 (m, 2H), 7.54 (m, 1H), 7.44 (s, 1H), 3.61 (s, 3H), 2.89 (m, 1H), 2.40 (m, 1H), 2.16 (m, 2H), 2.02 (m, 2H), 1.64 (m, 4H); MS: m/z 437.2 (M−1).

### Example 212

### 4-(5-(4-(2-Chlorobenzamido)phenyl)oxazol-2-yl)cyclohexanecarboxylic acid

The compound of example 212 was prepared analogous to the compound of example 15 by hydrolysis of the compound of example 211. Yield: 75%; ¹H NMR (DMSO-d₆, 300 MHz): δ 12.12 (bs, 1H), 10.65 (s, 1H), 7.82 (d, 2H), 7.67 (d, 2H), 7.62 (m, 2H), 7.55 (m, 2H), 7.46 (s, 1H), 2.84 (m, 1H), 2.27 (m, 1H), 2.16 (m, 2H), 2.02 (m, 2H), 1.58 (m, 4H); MS: m/z 425.1 (M+1).

### Example 213

### Methyl 4-(5-(4-(4-tert-butylbenzamido)phenyl)oxazol-2-yl)cyclohexane carboxylate

The compound of example 213 was prepared analogous to the compound of example 14 by reaction of the compound of example 202 with 4-(t-butyl)benzoyl chloride. Yield: 60%; ¹H NMR (DMSO-d₆, 300 MHz): δ 10.33 (s, 1H), 8.02 (m, 2H), 7.92 (m, 2H), 7.67 (d, 2H), 7.56 (d, 2H), 7.45 (s, 1H), 3.61 (s, 3H), 2.85 (m, 1H), 2.41 (m, 1H), 2.16 (m, 2H), 2.02 (m, 2H), 1.60 (m, 4H), 1.31 (s, 9H); MS: m/z 461.2 (M+1).

### Example 214

### 4-(5-(4-(4-Tert-butylbenzamido)phenyl)oxazol-2-yl)cyclohexanecarboxylic acid

The compound of example 214 was prepared analogous to the compound of example 15 by hydrolysis of the compound of example 213. Yield: 77%; ¹H NMR (DMSO-d₆, 300 MHz): δ 12.12 (s, 1H), 10.30 (s, 1H), 7.91 (d, 2H), 7.88 (d, 2H), 7.67 (d, 2H), 7.57 (d, 2H), 7.45 (s, 1H), 2.87 (m, 1H), 2.31 (m, 1H), 2.16 (m, 2H), 2.02 (m, 2H), 1.63 (m, 4H), 1.32 (s, 9H); MS: m/z 447.2 (M+1).

### Example 215

### (Z)—N′-hydroxy-4-nitrobenzimidamide

To a solution of 4-nitro benzonitrile (25 g, 0.168 mol) in EtOH (250 mL) was added hydroxylamine hydrochloride (17.60 g, 0.253 mol) and potassium carbonate (34.95 g, 0.253 mol) and refluxed for 8-9 h. The solvent was removed and the residue obtained was dissolved in ethyl acetate, washed with water and brine, dried over anhydrous sodium sulphate and concentrated to afford the title compound.

Yield: 29 g (95%); ¹H NMR (DMSO-d₆, 300 MHz): δ 10.13 (s, 1H), 8.25 (d, 2H), 7.95 (d, 2H), 6.09 (s, 2H), 3.20 (m, 1H), 2.45 (m, 1H), 2.22 (m, 2H), 2.05 (m, 2H), 1.69 (m, 4H); MS: m/z 181 (M+1).

### Example 216

### (1r,4r-Methyl 4-(3-(4-nitrophenyl)-1,2,4-oxadiazol-5-yl)cyclohexane carboxylate

To a suspension of the compound of example 129 (500 mg, 2.688 mmol) in dichloromethane (7.5 mL) was added N,N′-carbonyldiimidazole (655 mg, 4.032 mmol) at room temperature. The reaction mixture was stirred at room temperature for 1 h and the compound of example 215 (866 mg, 4.78 mmol) was added, followed by stirring at room temperature for 8 h. The mixture was concentrated, diluted with toluene (7.5 mL) and refluxed for 16 h. The reaction mixture was cooled to room temperature and diluted with ethyl acetate. The organic layer was washed with water and brine, dried over anhydrous sodium sulphate and concentrated to obtain a crude residue, which was purified by column chromatography (silicagel, ethyl acetate in petroleum ether) to afford the title compound. Yield: 700 mg (50%); ¹H NMR (DMSO-d₆, 300 MHz): δ 8.42 (d, 2H), 8.27 (d, 2H), 3.62 (s, 3H), 3.20 (m, 1H), 2.45 (m, 1H), 2.22 (m, 2H), 2.05 (m, 2H), 1.69 (m, 4H); MS: m/z 332 (M+1).

### Example 217

### (1r,4r)-Methyl 4-(3-(4-aminophenyl)-1,2,4-oxadiazol-5-yl)cyclohexane carboxylate

The compound of example 217 was prepared analogous to the compound of example 5 by reduction of the compound of example 216. Yield: 73%; ¹H NMR (DMSO-d₆, 300 MHz): δ 7.65 (d, 2H), 6.64 (d, 2H), 5.74 (s, 2H), 3.61 (s, 3H), 3.02 (m, 1H), 2.43 (m, 1H), 2.15 (m, 2H), 2.03 (m, 2H), 1.63 (m, 4H); MS: m/z 301 (M+1).

### Example 218

### (1r,4r)-Methyl 4-(3-(4-(3-(2-chlorophenyl)ureido)phenyl)-1,2,4-oxadiazol-5-yl)cyclohexanecarboxylate

The compound of example 218 was prepared analogous to the compound of example 6 by reaction of the compound of example 217 with 1-chloro-2-isocyanato benzene.

Yield: 96%; ¹H NMR (DMSO-d₆, 300 MHz): δ 9.74 (s, 1H), 8.41 (s, 1H), 8.18 (d, 1H), 7.95 (d, 2H), 7.66 (d, 2H), 7.49 (d, 1H), 7.32 (m, 1H), 7.08 (m, 1H), 3.61 (s, 3H), 3.09 (m, 1H), 2.44 (m, 1H), 2.19 (m, 2H), 2.03 (m, 2H), 1.67 (m, 4H); MS: m/z 455 (M+1).

### Example 219

### (1r,4r)-4-(3-(4-(3-(2-Chlorophenyl)ureido)phenyl)-1,2,4-oxadiazol-5-yl)cyclo hexanecarboxylic acid

The compound of example 219 was prepared analogous to the compound of example 7 by hydrolysis of the compound of example 218. Yield: 83%; ¹H NMR (DMSO-d₆, 300 MHz): δ 12.17 (s, 1H), 9.74 (s, 1H), 8.41 (s, 1H), 8.17 (d, 1H), 7.95 (d, 2H), 7.66 (d, 2H), 7.49 (d, 1H), 7.34 (t, 1H), 7.08 (t, 1H), 3.11 (m, 1H), 2.34 (m, 1H), 2.18 (m, 2H), 2.03 (m, 2H), 1.65 (m, 4H); MS: m/z 441 (M+1).

### Example 220

### (1r,4r)-Methyl 4-(3-(4-(3-(2,4-difluorophenyl)ureido)phenyl)-1,2,4-oxadiazol-5-yl)cyclohexanecarboxylate

The compound of example 220 was prepared analogous to the compound of example 6 by reaction of the compound of example 217 with 2,4-difluoro-1-isocyanato benzene.

Yield: 93%; ¹H NMR (DMSO-d₆, 300 MHz): δ 9.35 (s, 1H), 8.60 (s, 1H), 8.12 (m, 1H), 7.93 (d, 2H), 7.64 (d, 2H), 7.37 (m, 1H), 7.09 (m, 1H), 3.61 (s, 3H), 3.12 (m, 1H), 2.43 (m, 1H), 2.15 (m, 2H), 2.00 (m, 2H), 1.66 (m, 4H); MS: m/z 457 (M+1).

### Example 221

### (1r,4r)-4-(3-(4-(3-(2,4-Difluorophenyl)ureido)phenyl)-1,2,4-oxadiazol-5-yl)cyclohexanecarboxylic acid

The compound of example 221 was prepared analogous to the compound of example 7 by hydrolysis of the compound of example 220. Yield: 90%; ¹H NMR (DMSO-d₆, 300 MHz): δ 12.25 (s, 1H), 9.36 (s, 1H), 8.61 (s, 1H), 8.12 (m, 1H), 7.93 (d, 2H), 7.64 (d, 2H), 7.37 (m, 1H), 7.09 (m, 1H), 3.11 (m, 1H), 2.34 (m, 1H), 2.18 (m, 2H), 2.04 (m, 2H), 1.69 (m, 4H); MS: m/z 442 (M+1).

### Example 222

### (1r,4r)-Methyl 4-(3-(4-(3-p-tolylureido)phenyl)-1,2,4-oxadiazol-5-yl)cyclo hexane carboxylate

The compound of example 222 was prepared analogous to the compound of example 6 by reaction of the compound of example 217 with 1-isocyanato-4-methyl benzene.

Yield: 93%; ¹H NMR (DMSO-d₆, 300 MHz): δ 8.98 (s, 1H), 8.66 (s, 1H), 7.91 (d, 2H), 7.63 (d, 2H), 7.36 (d, 2H), 7.11 (d, 2H), 3.61 (s, 3H), 3.19 (m, 1H), 2.43 (m, 1H), 2.19 (m, 2H), 2.04 (m, 2H), 1.66 (m, 4H); MS: m/z 434 (M+1).

### Example 223

### (1r,4r)-4-(3-(4-(3-p-Tolyl ureido)Phenyl)-1,2,4-oxadiazol-5-yl)cyclohexane carboxylic acid

The compound of example 223 was prepared analogous to the compound of example 7 by hydrolysis of the compound of example 222. Yield: 78%; ¹H NMR (DMSO-d₆, 300 MHz): δ 12.18 (s, 1H), 8.98 (s, 1H), 8.66 (s, 1H), 7.91 (d, 2H), 7.63 (d, 2H), 7.36 (d, 2H), 7.11 (d, 2H), 3.07 (m, 1H), 2.31 (m, 1H), 2.1 (m, 2H), 2.04 (m, 2H), 1.65 (m, 4H); MS: m/z 420 (M+1).

### Example 224

### (1r,4r)-Methyl 4-(3-(4-(3-(3-chlorophenyl)ureido)phenyl)-1,2,4-oxadiazol-5-yl)cyclohexanecarboxylate

The compound of example 224 was prepared analogous to the compound of example 6 by reaction of the compound of example 217 with 1-chloro-3-isocyanato benzene.

Yield: 88%; ¹H NMR (DMSO-d₆, 300 MHz): δ 9.11 (s, 1H), 8.99 (s, 1H), 7.93 (d, 1H), 7.72 (s, 1H), 7.65 (d, 2H), 7.32 (m, 2H), 7.05 (d, 1H), 3.61 (s, 3H), 3.12 (m, 1H), 2.44 (m, 1H), 2.19 (m, 2H), 2.04 (m, 2H), 1.71 (m, 4H); MS: m/z 455 (M+1).

### Example 225

### (1r,4r)-4-(3-(4-(3-(3-Chlorophenyl)ureido)phenyl)-1,2,4-oxadiazol-5-yl)cyclo hexanecarboxylic acid

The compound of example 225 was prepared analogous to the compound of example 7 by hydrolysis of the compound of example 224. Yield: 83%; ¹H NMR (DMSO-d₆, 300 MHz): δ 12.17 (s, 1H), 9.41 (s, 1H), 9.29 (s, 1H), 7.93 (d, 2H), 7.74 (s, 1H), 7.66 (d, 2H), 7.32 (d, 2H), 7.05 (m, 1H), 3.11 (m, 1H), 2.33 (m, 1H), 2.18 (m, 2H), 2.03 (m, 2H), 1.69 (m, 4H); MS: m/z 441 (M+1).

### Example 226

### (1r,4r)-Methyl 4-(3-(4-(3-(4-chloro-2-phenoxyphenyl)ureido)phenyl)-1,2,4-oxadiazol-5-yl)cyclohexanecarboxylate

The compound of example 226 was prepared analogous to the compound of example 6 by reaction of the compound of example 217 with 4-chloro-1-isocyanato-2-phenoxy benzene. Yield: 44%; ¹H NMR (DMSO-d₆, 300 MHz): δ 9.68 (s, 1H), 8.76 (s, 1H), 8.40 (d, 1H), 7.94 (s, 2H), 7.63 (d, 2H), 7.47 (t, 2H), 7.22 (t, 1H), 7.11 (d, 2H), 7.03 (dd, 1H), 6.85 (d, 1H), 3.61 (s, 3H), 3.13 (m, 1H), 2.18 (m, 2H), 2.03 (m, 2H), 1.71 (m, 4H); MS: m/z 547 (M+1).

### Example 227

### (1r,4r)-4-(3-(4-(3-(4-Chloro-2-phenoxyphenyl)ureido)phenyl)-1,2,4-oxadiazol-5-yl)cyclohexanecarboxylic acid

The compound of example 227 was prepared analogous to the compound of example 7 by hydrolysis of the compound of example 226. Yield: 90%; ¹H NMR (DMSO-d₆, 300 MHz): δ 12.19 (s, 1H), 9.75 (s, 1H), 8.78 (s, 1H), 8.39 (d, 1H), 7.93 (d, 2H), 7.63 (s, 2H), 7.46 (t, 2H), 7.22 (t, 2H), 7.11 (d, 2H), 7.03 (dd, 1H), 6.85 (d, 1H), 3.07 (m, 1H), 2.18 (m, 2H), 2.04 (m, 2H), 1.65 (m, 4H); MS: m/z 533 (M+1).

### Example 228

### (1r,4r)-Methyl 4-(3-(4-(4-tert-butylbenzamido)phenyl)-1,2,4-oxadiazol-5-yl)cyclohexanecarboxylate

The compound of example 228 was prepared analogous to the compound of example 14 by reaction of the compound of example 217 with 4-(t-butyl)benzoyl chloride. Yield: 86%; ¹H NMR (DMSO-d₆, 300 MHz): δ 10.45 (s, 1H), 7.98 (s, 4H), 7.92 (d, 2H), 7.58 (d, 2H), 3.62 (s, 3H), 3.10 (m, 1H), 2.45 (m, 1H), 2.19 (m, 2H), 2.04 (m, 2H), 1.67 (m, 4H), 1.33 (s, 9H); MS: m/z 462 (M+1).

### Example 229

### (1r,4r)-4-(3-(4-(4-tert-Butylbenzamido)phenyl)-1,2,4-oxadiazol-5-yl)cyclo hexane carboxylic acid

The compound of example 229 was prepared analogous to the compound of example 15 by hydrolysis of the compound of example 228. Yield: 83%; ¹H NMR (DMSO-d₆, 300 MHz): δ 12.17 (s, 1H), 10.45 (s, 1H), 7.98 (s, 4H), 7.92 (d, 2H), 7.58 (d, 2H), 3.12 (m, 1H), 2.35 (m, 1H), 2.20 (m, 2H), 2.05 (m, 2H), 1.70 (m, 4H), 1.33 (s, 9H); MS: m/z 448 (M+1).

### Example 230

### (1r,4r)-Methyl 4-(3-(4-biphenyl-4-ylcarboxamidophenyl)-1,2,4-oxadiazol-5-yl)cyclohexanecarboxylate

The compound of example 230 was prepared analogous to the compound of example 14 by reaction of the compound of example 217 with 4-phenyl benzoyl chloride. Yield: 88%; ¹H NMR (DMSO-d₆, 300 MHz): δ 10.58 (s, 1H), 8.10 (d, 2H), 8.04 (d, 4H), 7.87 (d, 2H), 7.78 (d, 2H), 7.54 (t, 2H), 7.45 (t, 1H), 3.62 (s, 3H), 2.45 (m, 1H), 2.21 (m, 2H), 2.05 (m, 2H), 1.68 (m, 4H); MS: m/z 482 (M+1).

### Example 231

### (1r,4r)-4-(3-(4-Biphenyl-4-ylcarboxamidophenyl)-1,2,4-oxadiazol-5-yl)cyclo hexanecarboxylic acid

The compound of example 231 was prepared analogous to the compound of example 15 by hydrolysis of the compound of example 230. Yield: 93%; ¹H NMR (DMSO-d₆, 300 MHz): δ 12.12 (s, 1H), 10.68 (s, 1H), 8.10 (d, 2H), 8.01 (d, 4H), 7.87 (d, 2H), 7.78 (d, 2H), 7.54 (t, 2H), 7.45 (t, 1H), 3.13 (s, 3H), 2.35 (m, 1H), 2.19 (m, 2H), 2.04 (m, 2H), 1.71 (m, 4H); MS: m/z 468 (M+1).

### Example 232

### (1r,4r)-Methyl 4-(3-(4-(4-(trifluoromethoxy)benzamido)phenyl)-1,2,4-oxadiazol-5-yl)cyclohexanecarboxylate

The compound of example 232 was prepared analogous to the compound of example 14 by reaction of the compound of example 217 with 4-trifluoromethyl benzoyl chloride.

Yield: 89%; ¹H NMR (DMSO-d₆, 300 MHz): δ 10.75 (s, 1H), 8.13 (d, 2H), 8.02 (d, 4H), 7.56 (d, 2H), 3.62 (s, 3H), 3.14 (m, 1H), 2.49 (m, 1H), 2.20 (m, 2H), 2.05 (m, 2H), 1.68 (m, 4H); MS: m/z 488 (M−1).

### Example 233

### (1r,4r)-4-(3-(4-(4-(Trifluoromethoxy)benzamido)phenyl)-1,2,4-oxadiazol-5-yl)cyclohexanecarboxylic acid

The compound of example 233 was prepared analogous to the compound of example 15 by hydrolysis of the compound of example 232. Yield: 94%; ¹H NMR (DMSO-d₆, 300 MHz): δ 12.35 (s, 1H), 10.62 (s, 1H), 8.11 (d, 2H), 8.02 (d, 4H), 7.57 (d, 2H), 3.16 (m, 1H), 2.34 (m, 1H), 2.20 (m, 2H), 2.05 (m, 2H), 1.66 (m, 4H); MS: m/z 474 (M−1).

### Example 234

### Methyl 4-(5-(4-(3-(3,5-difluorophenyl)ureido)phenyl)thiazol-2-yl)-2,2-dimethyl butanoate

The compound of example 234 was prepared analogous to the compound of example 6 by reaction of the compound of example 86 with 3,5-difluoro-1-isocyanato benzene.

Yield: 89%; ¹H NMR (DMSO-d₆, 300 MHz): δ 9.12 (s, 1H), 9.01 (s, 1H), 7.94 (s, 1H), 7.57-7.49 (dd, 4H), 7.21-7.17 (dd, 2H), 6.83-6.77 (m, 1H), 3.62 (s, 3H), 2.9 (m, 2H), 1.97 (m, 2H), 1.20 (s, 6H); MS: m/z 460.2 (M+1).

### Example 235

### 4-(5-(4-(3-(3,5-Difluorophenyl)ureido)phenyl)thiazol-2-yl)-2,2-dimethyl butanoic acid

The compound of example 235 was prepared analogous to the compound of example 7 by hydrolysis of the compound of example 234. Yield: 91%; ¹H NMR (DMSO-d₆, 300 MHz): δ 12.31 (bs, 1H), 9.19 (s, 1H), 8.55 (s, 1H), 8.11-8.03 (m, 1H), 7.94 (s, 1H), 7.56-7.49 (dd, 4H), 7.36-7.28 (m, 1H), 7.08-7.02 (m, 1H), 2.91 (m, 2H), 1.93 (m, 2H), 1.17 (s, 6H); MS: m/z 446 (M+1).

### Example 235A

### Sodium salt of 4-(5-(4-(3-(3,5-difluorophenyl)ureido)phenyl)thiazol-2-yl)-2,2-dimethylbutanoate

The compound of example 235A is prepared analogous to the compound of example 90A by reaction of the compound of example 235 with 1N NaOH solution. Yield: 76%;

¹H NMR (DMSO-d₆, 300 MHz): δ 12.95 (s, 1H), 12.66 (s, 1H), 7.88 (s, 1H), 7.83-7.81 (d, 2H), 7.55 (d, 2H), 7.38 (d, 2H), 6.64 (m, 1H), 2.96 (m, 2H), 1.91 (m, 2H), 1.14 (s, 6H); MS: m/z 446 (M+1).

### Example 236

### Methyl 2,2-dimethyl-4-(5-(4-(3-(2,4,5-trifluorophenyl)ureido)phenyl)thiazol-2-yl)butanoate

The compound of example 236 was prepared analogous to the compound of example 6 by reaction of the compound of example 86 with 2,4,5-trifluoro-1-isocyanato benzene. Yield: 81%; ¹H NMR (DMSO-d₆, 300 MHz): δ 9.22 (s, 1H), 8.74 (s, 1H), 8.24-8.14 (m, 1H), 7.94 (s, 1H), 7.67-7.64 (m, 1H), 7.60-7.48 (dd, 4H), 3.62 (s, 3H), 2.89 (m, 2H), 1.97 (m, 2H), 1.19 (s, 6H); MS: m/z 478 (M+1).

### Example 237

### 2,2-Dimethyl-4-(5-(4-(3-(2,4,5-trifluorophenyl)ureido)phenyl)thiazol-2-yl)butanoic acid

The compound of example 237 was prepared analogous to the compound of example 7 by hydrolysis of the compound of example 236. Yield: 82%; ¹H NMR (DMSO-d₆, 300 MHz): δ 12.27 (bs, 1H), 9.47 (s, 1H), 8.96 (s, 1H), 8.22-8.13 (m, 1H), 7.94 (s, 1H), 7.68-7.64 (m, 1H), 7.62-7.50 (dd, 4H), 2.92 (m, 2H), 1.93 (m, 2H), 1.16 (s, 6H); MS: m/z 464.1 (M+1).

### Example 237A

### Sodium salt of 2,2-dimethyl-4-(5-(4-(3-(2,4,5-trifluorophenyl)ureido)phenyl)thiazol-2-yl)butanoate

The compound of example 237A is prepared analogous to the compound of example 90A by reaction of the compound of example 237 with 1N NaOH solution.

Yield: 86%; ¹H NMR (DMSO-d₆, 300 MHz): δ 12.47 (s, 1H), 11.84 (s, 1H), 7.89 (m, 1H), 7.85 (s, 1H), 7.78-7.75 (d, 2H), 7.51-7.48 (d, 2H), 7.45 (m, 1H), 2.90 (m, 2H), 1.86 (m, 2H), 1.07 (s, 6H); MS: m/z 464.1 (M+1).

### Example 238

### Methyl 2,2-dimethyl-4-(5-(4-(piperidine-1-carboxamido)phenyl)thiazol-2-yl)butanoate

The compound of example 86 (1.2 g, 3.94 mmol) was dissolved in dichloromethane (24 mL). To this triphosgene (0.585 g, 1.971 mmol) was added followed by triethylamine (0.824 mL, 5.91 mmol) and stirred at room temperature for 30 min. To this piperidine (77 mg, 0.908 mmol) was added and stirred at room temperature for 24 h. The solvent was evaporated to obtain a residue which was purified by column chromatography (silica gel, 20% ethyl acetate in chloroform) to obtain a solid which was crystallised in chloroform-petroleum ether to afford the title compound. Yield: 185 mg (73%); ¹H NMR (DMSO-d₆, 300 MHz): δ 8.58 (s, 1H), 7.90 (s, 1H), 7.54-7.45 (dd, 4H), 3.62 (s, 3H), 3.48-3.41 (m, 4H), 2.88 (m, 2H), 1.96 (m, 2H), 1.56 (m, 2H), 1.49 (m, 4H), 1.19 (s, 6H); MS: m/z 416.2 (M+1).

### Example 239

### 2,2-Dimethyl-4-(5-(4-(piperidine-1-carboxamido)phenyl)thiazol-2-yl)butanoic acid

The compound of example 239 was prepared analogous to the compound of example 7 by hydrolysis of the compound of example 238. Yield: 61%; ¹H NMR (DMSO-D6, 300 MHz) δ 12.28 (bs, 1H), 8.58 (s, 1H), 7.93 (s, 1H), 7.54-7.45 (dd, 4H), 3.42 (m, 4H), 2.90 (m, 2H), 1.93 (m, 2H), 1.56 (m, 2H), 1.49 (m, 4H), 1.16 (s, 6H); MS: m/z 402 (M+1).

### Example 240

### Methyl 2,2-dimethyl-4-(5-(4-(morpholine-4-carboxamido)phenyl)thiazol-2-yl)butanoate

The compound of example 240 was prepared analogous to the compound of example 238 by reaction of the compound 86 with morpholine. Yield: 49%; ¹H NMR (DMSO-d₆, 300 MHz): δ 8.68 (s, 1H), 7.92 (s, 1H), 7.55-7.47 (dd, 4H), 3.62 (s, 3H), 3.59 (m, 4H), 3.44-3.43 (m, 4H), 2.89 (m, 2H), 1.96 (m, 2H), 1.19 (s, 6H); MS: m/z 418.2 (M+1).

### Example 241

### 2,2-Dimethyl-4-(5-(4-(morpholine-4-carboxamido)phenyl)thiazol-2-yl)butanoic acid

The compound of example 241 was prepared analogous to the compound of example 7 by hydrolysis of the compound of example 240. Yield: 85%; ¹H NMR (DMSO-d₆, 300 MHz): δ 12.31 (bs, 1H), 8.67 (s, 1H), 7.91 (s, 1H), 7.55-7.47 (dd, 4H), 3.62-3.59 (m, 4H), 3.44-3.41 (m, 4H), 2.91 (m, 2H), 1.93 (m, 2H), 1.16 (s, 6H); MS: m/z 404.1 (M+1).

### Example 242

### Methyl 2,2-dimethyl-4-(5-(4-(4-methylpiperazine-1-carboxamido)phenyl)thiazol-2-yl)butanoate

The compound of example 242 was prepared analogous to the compound of example 238 by reaction of the compound 86 with N-methylpiperazine. Yield: 69%;

¹H NMR (DMSO-d₆, 300 MHz): δ 8.66 (s, 1H), 7.91 (s, 1H), 7.54-7.46 (dd, 4H), 3.62 (s, 3H), 3.45 (m, 4H), 2.89 (m, 2H), 2.35 (m, 4H), 2.22 (s, 3H), 1.96 (m, 2H), 1.19 (s, 6H); MS: m/z 431.2 (M+1).

### Example 243

### 2,2-Dimethyl-4-(5-(4-(4-methylpiperazine-1-carboxamido)phenyl)thiazol-2-yl)butanoic acid hydrochloride

The compound of example 243 was prepared analogous to the compound of example 7 by hydrolysis of the compound of example 242. Yield: 85%; ¹H NMR (DMSO-d₆, 300 MHz): δ 12.3 (bs, 1H), 11.15 (bs, 1H), 9.07 (s, 1H), 7.92 (s, 1H), 7.55-7.52 (dd, 4H), 4.30-4.26 (m, 2H), 3.19 (m, 2H), 3.02 (m, 4H), 2.91 (m, 2H), 2.75 (s, 3H), 1.92 (m, 2H), 1.16 (s, 6H); MS: m/z 417 (M+1).

### Example 244

### Methyl 4-(5-(4-(3-(2,3-dihydrobenzo[b][1,4]dioxin-6-yl)ureido)phenyl)thiazol-2-yl)-2,2-dimethylbutanoate

The compound of example 244 was prepared analogous to the compound of example 238 by reaction of the compound 86 with 2,3-dihydrobenzo[b][1,4]dioxin-6-amine. Yield: 14%; ¹H NMR (DMSO-d₆, 300 MHz): δ 8.74 (s, 1H), 8.50 (s, 1H), 7.92 (s, 1H), 7.54-7.47 (dd, 4H), 7.09 (d, 1H), 6.82-6.74 (m, 2H), 4.21-4.19 (m, 4H), 3.62 (s, 3H), 2.89 (m, 2H), 1.97 (m, 2H), 1.20 (s, 6H); MS: m/z 482.2 (M+1).

### Example 245

### 4-(5-(4-(3-(2,3-Dihydrobenzo[b][1,4]dioxin-6-yl)ureido)phenyl)thiazol-2-yl)-2,2-dimethylbutanoic acid

The compound of example 245 was prepared analogous to the compound of example 7 by hydrolysis of the compound of example 244. Yield: 86%; ¹H NMR (DMSO-d₆, 300 MHz): δ 12.29 (bs, 1H), 8.74 (s, 1H), 8.50 (s, 1H), 7.92 (s, 1H), 7.54-7.46 (dd, 4H), 7.09 (d, 1H), 6.78-6.74 (m, 2H), 4.21-4.19 (m, 4H), 2.91 (m, 2H), 1.93 (m, 2H), 1.23 (s, 6H); MS: m/z 468 (M+1).

### Example 246

### Methyl 4-(5-(4-(3-(1H-tetrazol-5-yl)ureido)phenyl)thiazol-2-yl)-2,2-dimethyl butanoate

The compound of example 246 was prepared analogous to the compound of example 238 by reaction of the compound 86 with 1H-tetrazol-5-amine. Yield: 40%; ¹H NMR (DMSO-d₆, 300 MHz): δ 15.66 (bs, 1H), 10.57 (s, 1H), 9.17 (s, 1H), 7.97 (s, 1H), 7.65-7.53 (dd, 4H), 3.62 (s, 3H), 2.90 (m, 2H), 1.97 (m, 2H), 1.20 (s, 6H); MS: m/z 416.2 (M+1).

### Example 247

### 4-(5-(4-(3-(1H-tetrazol-5-yl)ureido)phenyl)thiazol-2-yl)-2,2-dimethylbutanoic acid

The compound of example 247 was prepared analogous to the compound of example 7 by hydrolysis of the compound of example 246. Yield: 91%; ¹H NMR (DMSO-d₆, 300 MHz): δ 15.67 (bs, 1H), 12.29 (bs, 1H), 10.57 (s, 1H), 9.20 (s, 1H), 7.97 (s, 1H), 7.65-7.53 (dd, 4H), 2.92 (m, 2H), 1.94 (m, 2H), 1.17 (s, 6H); MS: m/z 402 (M+1).

### Example 248

### Methyl 4-(5-(4-(3-(2-methoxyethyl)ureido)phenyl)thiazol-2-yl)-2,2-dimethyl butanoate

The compound of example 248 was prepared analogous to the compound of example 238 by reaction of the compound 86 with 2-methoxyethanamine. Yield: 66%; ¹H NMR (DMSO-d₆, 300 MHz) δ 8.69 (s, 1H), 7.89 (s, 1H), 7.48-7.41 (dd, 4H), 6.24-6.22 (t, 1H), 3.61 (s, 3H), 3.39-3.33 (m, 2H), 3.27 (s, 3H), 3.24-3.23 (m, 2H), 2.88 (m, 2H), 1.96 (m, 2H), 1.19 (s, 6H); MS: m/z 406.2 (M+1).

### Example 249

### 4-(5-(4-(3-(2-methoxyethyl)ureido)phenyl)thiazol-2-yl)-2,2-dimethylbutanoic acid

The compound of example 249 was prepared analogous to the compound of example 7 by hydrolysis of the compound of example 248. Yield: 76%; ¹H NMR (DMSO-d₆, 300 MHz): δ 12.29 (bs, 1H), 8.70 (s, 1H), 7.89 (s, 1H), 7.46-7.44 (dd, 4H), 6.24 (t, 1H), 3.37-3.33 (m, 4H), 3.27 (s, 3H), 2.90 (m, 2H), 1.92 (m, 2H), 1.16 (s, 6H); MS: m/z 392.2 (M+1).

### Example 250

### Methyl 4-(5-(4-(3-(2,3-dihydro-1H-inden-2-yl)ureido)phenyl)thiazol-2-yl)-2,2-dimethylbutanoate

The compound of example 250 was prepared analogous to the compound of example 238 by reaction of the compound 86 with 2,3-dihydro-1H-inden-2-amine hydrochloride.

Yield: 69%; ¹H NMR (DMSO-d₆, 300 MHz): δ 8.48 (s, 1H), 7.89 (s, 1H), 7.48-7.41 (dd, 4H), 7.27-7.24 (m, 2H), 7.17-7.14 (m, 2H), 6.51-6.49 (d, 1H), 4.44-4.42 (m, 1H), 3.62 (s, 3H), 3.23-3.15 (dd, 2H), 2.88 (m, 2H), 2.81-2.74 (dd, 2H), 1.96 (m, 2H), 1.19 (s, 6H); MS: m/z 464.2 (M+1).

### Example 251

### 4-(5-(4-(3-(2,3-Dihydro-1H-inden-2-yl)ureido)phenyl)thiazol-2-yl)-2,2-dimethyl butanoic acid

The compound of example 249 was prepared analogous to the compound of example 7 by hydrolysis of the compound of example 248. Yield: 90%; ¹H NMR (DMSO-d₆, 300 MHz): δ 12.30 (bs, 1H), 8.53 (s, 1H), 7.89 (s, 1H), 7.48-7.40 (dd, 4H), 7.26-7.23 (m, 2H), 7.17-7.14 (m, 2H), 6.55-6.52 (d, 1H), 4.45-4.39 (m, 1H), 3.23-3.15 (dd, 2H), 2.90 (m, 2H), 2.80-2.73 (dd, 2H), 1.92 (m, 2H), 1.16 (s, 6H); MS: m/z 450.2 (M+1).

### Example 252

### Methyl 4-(5-(4-(3-cyclohexyl-3-methyl ureido)phenyl)thiazol-2-yl)-2,2-dimethyl butanoate

The compound of example 252 was prepared analogous to the compound of example 238 by reaction of the compound 86 with N-methylcyclohexanamine. Yield: 62%; ¹H NMR (DMSO-d₆, 300 MHz) δ 8.33 (s, 1H), 7.91 (s, 1H), 7.55-7.45 (dd, 4H), 4.01 (m, 1H), 3.62 (s, 3H), 3.33-3.21 (m, 1H), 2.88 (m, 2H), 2.81 (s, 3H), 1.96 (m, 2H), 1.78-1.74 (m, 2H), 1.65-1.56 (m, 2H), 1.50-1.34 (m, 5H), 1.19 (s, 6H); MS: m/z 444.2 (M+1).

### Example 253

### 4-(5-(4-(3-cyclohexyl-3-methylureido)phenyl)thiazol-2-yl)-2,2-dimethyl butanoic acid

The compound of example 253 was prepared analogous to the compound of example 7 by hydrolysis of the compound of example 252. Yield: 87%; ¹H NMR (DMSO-d₆, 300 MHz): δ 12.30 (bs, 1H), 8.33 (s, 1H), 7.91 (s, 1H), 7.55-7.45 (dd, 4H), 4.00 (m, 1H), 3.34-3.31 (m, 1H), 2.90 (m, 2H), 2.81 (s, 3H), 1.95-1.90 (m, 2H), 1.78-1.74 (m, 2H), 1.62-1.50 (m, 2H), 1.46-1.29 (m, 5H), 1.16 (s, 6H); MS: m/z 430.2 (M+1).

### Example 254

### Methyl 2,2-dimethyl-4-(5-(4-(3-(3,4,5-trifluorophenyl)ureido)phenyl)thiazol-2-yl)butanoate

The compound of example 254 was prepared analogous to the compound of example 238 by reaction of the compound 86 with 3,4,5-trifluoroaniline. Yield: 64%; ¹H NMR (DMSO-d₆, 300 MHz) δ 9.07 (s, 1H), 8.04 (s, 1H), 7.94 (s, 1H), 7.56-7.49 (dd, 4H), 7.42-7.36 (dd, 2H), 3.62 (s, 3H), 2.90 (m, 2H), 1.97 (m, 2H), 1.20 (s, 6H); MS: m/z 478.1 (M+1).

### Example 255

### 2,2-dimethyl-4-(5-(4-(3-(3,4,5-trifluorophenyl)ureido)phenyl)thiazol-2-yl)butanoic acid

The compound of example 255 was prepared analogous to the compound of example 7 by hydrolysis of the compound of example 254. Yield: 90%; ¹H NMR (DMSO-d₆, 300 MHz) δ 12.30 (bs, 1H), 9.12 (s, 1H), 9.07 (s, 1H), 7.94 (s, 1H), 7.56-7.48 (dd, 4H), 7.41-7.36 (dd, 2H), 2.91 (m, 2H), 1.93 (m, 2H), 1.16 (s, 6H); MS: m/z 464.1 (M+1).

### Example 255A

### Sodium salt of 2,2-dimethyl-4-(5-(4-(3-(3,4,5-trifluorophenyl)ureido) phenyl)thiazol-2-yl)butanoate

The compound of example 255A is prepared analogous to the compound of example 90A by reaction of the compound of example 255 with 1N NaOH solution.

Yield: 80%; ¹H NMR (DMSO-d₆, 300 MHz): δ 11.49 (bs, 2H), 7.88 (s, 1H), 7.68-7.65 (d, 2H), 7.53-7.50 (d, 2H), 7.48-7.42 (m, 2H), 2.92 (m, 2H), 1.89 (m, 2H), 1.13 (s, 6H); MS: m/z 464.1 (M+1).

### Example 256

### Methyl 2,2-dimethyl-4-(5-(4-(3-(2-(piperidin-1-yl)ethyl)ureido)phenyl)thiazol-2-yl)butanoate

The compound of example 256 was prepared analogous to the compound of example 238 by reaction of the compound 86 with 2-(piperidin-1-yl)ethanamine. Yield: 41%; ¹H NMR (DMSO-d₆, 300 MHz): δ 9.93 (bs, 1H), 9.30 (s, 1H), 7.98 (s, 1H), 7.48 (m, 4H), 6.82-6.79 (m, 1H), 3.61 (s, 3H), 3.50-3.48 (m, 3H), 3.12-3.06 (m, 4H), 2.87 (m, 2H), 1.98 (m, 2H), 1.83-1.76 (m, 4H), 1.23-1.21 (m, 2H), 1.19 (s, 6H); MS: m/z 459.2 (M+1).

### Example 257

### 2,2-Dimethyl-4-(5-(4-(3-(2-(piperidin-1-yl)ethyl)ureido)phenyl)thiazol-2-yl)butanoic acid

The compound of example 257 was prepared analogous to the compound of example 7 by hydrolysis of the compound of example 256. Yield: 41%; ¹H NMR (DMSO-d₆, 300 MHz) δ 12.24 (bs, 1H), 9.73 (s, 1H), 9.20 (s, 1H), 7.90 (s, 1H), 7.48 (m, 4H), 6.70 (m, 1H), 3.48-3.46 (m, 3H), 3.09 (m, 2H), 2.90 (m, 4H), 1.95 (m, 2H), 1.75 (m, 4H), 1.37 (m, 2H), 1.16 (s, 6H); MS: m/z 445.2 (M+1).

### Example 258

### Methyl 4-(5-(4-(3-benzyl ureido)phenyl)thiazol-2-yl)-2,2-dimethyl butanoate

The compound of example 258 was prepared analogous to the compound of example 238 by reaction of the compound 86 with phenylmethanamine. Yield: 41%; ¹H NMR (DMSO-d₆, 300 MHz) δ 8.74 (s, 1H), 7.89 (s, 1H), 7.50-7.44 (dd, 4H), 7.36-7.26 (dd, 4H), 7.24-7.22 (m, 1H), 6.69-6.65 (t, 1H), 4.31-4.29 (d, 2H), 3.61 (s, 3H), 2.88 (m, 2H), 1.96 (m, 2H), 1.19 (s, 6H); MS: m/z 438.2 (M+1).

### Example 259

### 4-(5-(4-(3-Benzylureido)phenyl)thiazol-2-yl)-2,2-dimethylbutanoic acid

The compound of example 259 was prepared analogous to the compound of example 7 by hydrolysis of the compound of example 258. Yield: 52%; ¹H NMR (DMSO-d₆, 300 MHz) δ 12.29 (bs, 1H), 8.76 (s, 1H), 7.90 (s, 1H), 7.50-7.44 (dd, 4H), 7.36-7.29 (dd, 4H), 7.27-7.22 (m, 1H), 6.70-6.66 (t, 1H), 4.31-4.29 (d, 2H), 2.89 (m, 2H), 1.92 (m, 2H), 1.16 (s, 6H); MS: m/z 424.2 (M+1).

### Example 260

### Methyl 4-(5-(4-(4,4-difluoropiperidine-1-carboxamido)phenyl)thiazol-2-yl)-2,2-dimethylbutanoate

The compound of example 260 was prepared analogous to the compound of example 238 by reaction of the compound 86 with 4,4-difluoropiperidine hydrochloride. Yield: 52%; ¹H NMR (DMSO-d₆, 300 MHz) δ 8.83 (s, 1H), 7.92 (s, 1H), 7.54-7.47 (dd, 4H), 3.61 (s, 3H), 3.59-3.56 (m, 4H), 2.88 (m, 2H), 2.03-1.93 (m, 6H), 1.19 (s, 6H); MS: m/z 452.2 (M+1).

### Example 261

### 4-(5-(4-(4,4-Difluoropiperidine-1-carboxamido)phenyl)thiazol-2-yl)-2,2-dimethyl butanoic acid

The compound of example 261 was prepared analogous to the compound of example 7 by hydrolysis of the compound of example 260. Yield: 86%; ¹H NMR (DMSO-d₆, 300 MHz): δ 12.29 (bs, 1H), 8.83 (s, 1H), 7.92 (s, 1H), 7.51-7.48 (dd, 4H), 3.58 (m, 4H), 2.90 (m, 2H), 2.03-1.90 (m, 6H), 1.16 (s, 6H); MS: m/z 438.2 (M+1).

### Example 262

### Methyl 2,2-dimethyl-4-(5-(4-(4-phenylpiperidine-1-carboxamido)phenyl)thiazol-2-yl)butanoate

The compound of example 260 was prepared analogous to the compound of example 238 by reaction of the compound 86 with 4-phenylpiperidine. Yield: 37%; ¹H NMR (DMSO-d₆, 300 MHz): δ 8.68 (s, 1H), 7.91 (s, 1H), 7.57-7.54 (d, 2H), 7.50-7.47 (d, 2H), 7.33-7.25 (m, 4H), 7.21-7.19 (m, 1H), 4.30-4.25 (d, 2H), 3.62 (s, 3H), 2.91-2.86 (m, 4H), 2.74 (m, 1H), 1.96 (m, 2H), 1.82-1.79 (m, 2H), 1.58-1.55 (m, 2H), 1.19 (s, 6H); MS: m/z 492.2 (M+1).

### Example 263

### 2,2-Dimethyl-4-(5-(4-(4-phenylpiperidine-1-carboxamido)phenyl)thiazol-2-yl)butanoic acid

The compound of example 263 was prepared analogous to the compound of example 7 by hydrolysis of the compound of example 262. Yield: 93%; ¹H NMR (DMSO-d₆, 300 MHz): δ 8.71 (s, 1H), 7.90 (s, 1H), 7.57-7.54 (d, 2H), 7.49-7.46 (d, 2H), 7.30-7.27 (m, 4H), 7.22-7.19 (m, 1H), 4.30-4.26 (d, 2H), 2.98-2.84 (m, 4H), 2.73 (m, 1H), 1.88-1.78 (m, 4H), 1.59-1.55 (m, 2H), 1.12 (s, 6H); MS: m/z 478.2 (M+1).

### Example 264

### Methyl 2,2-dimethyl-4-(5-(4-(4-phenylpiperidine-1-carboxamido) phenyl)thiazol-2-yl)butanoate

The compound of example 264 was prepared analogous to the compound of example 238 by reaction of the compound of example 86 with 4-(aminomethyl)benzonitrile hydrochloride. Yield: 52%; ¹H NMR (DMSO-d₆, 300 MHz): δ 8.85 (s, 1H), 7.87 (s, 1H), 7.79-7.75 (d, 2H), 7.48-7.38 (m, 6H), 6.81-6.77 (t, 1H), 4.37-4.35 (d, 2H), 3.59 (s, 3H), 2.86 (m, 2H), 1.93 (m, 2H), 1.16 (s, 6H); MS: m/z 463.2 (M+1).

### Example 265

### 4-(5-(4-(3-(4-Cyanobenzyl)ureido)phenyl)thiazol-2-yl)-2,2-dimethylbutanoic acid

The compound of example 265 was prepared analogous to the compound of example 7 by hydrolysis of the compound of example 264. Yield: 77%; ¹H NMR (DMSO-d₆, 300 MHz) δ 12.21 (bs, 1H), 8.90 (s, 1H), 7.88 (s, 1H), 7.80-7.77 (d, 2H), 7.48-7.45 (m, 6H), 6.83 (t, 1H), 4.37-4.36 (d, 2H), 2.88 (m, 2H), 1.90 (m, 2H), 1.14 (s, 6H); MS: m/z 449.2 (M+1).

### Example 266

### Methyl 4-(5-(4-(3-(2-fluorophenyl)thioureido)phenyl)thiazol-2-yl)-2,2-dimethyl butanoate

To a solution of compound of example 86 (1 g, 3.29 mmol) in dichloromethane (10 mL) was added 1-fluoro-2-isothiocyanatobenzene (0.426 mL, 3.45 mmol) and stirred at room temperature for 24 h. The solvent was evaporated to obtain a residue which was purified by column chromatography (silica gel, 20% ethyl acetate in chloroform) to obtain a solid, which was crystallised in chloroform-petroleum ether to afford the title compound. Yield: 980 mg (65%); ¹H NMR (DMSO-d₆, 300 MHz): δ 10.08 (s, 1H), 9.56 (s, 1H), 8.00 (s, 1H), 7.63-7.58 (m, 5H), 7.28-7.22 (m, 2H), 7.20-7.16 (m, 1H), 3.62 (s, 3H), 2.91 (m, 2H), 1.98 (m, 2H), 1.20 (s, 6H); MS: m/z 458.1 (M+1).

### Example 267

### 4-(5-(4-(3-(2-Fluorophenyl)thioureido)phenyl)thiazol-2-yl)-2,2-dimethyl butanoic acid

The compound of example 267 was prepared analogous to the compound of example 7 by hydrolysis of the compound of example 266. Yield: 94%; ¹H NMR (DMSO-d₆, 300 MHz): δ 12.28 (bs, 1H), 10.12 (s, 1H), 9.59 (s, 1H), 8.01 (s, 1H), 7.63-7.53 (m, 5H), 7.29-7.22 (m, 2H), 7.20-7.16 (m, 1H), 2.93 (m, 2H), 1.95 (m, 2H), 1.17 (s, 6H); MS: m/z 444.1 (M+1).

### Example 268

### Methyl 4-(5-(4-(3-(2-fluorophenyl)guanidino)phenyl)thiazol-2-yl)-2,2-dimethyl butanoate

To a solution of the compound of example 266 (250 mg, 0.546 mmol) in 7N methanolic ammonia (7.80 mL, 54.6 mmol) was added mercuric oxide yellow (296 mg, 1.366 mmol) and the reaction mixture was stirred at room temperature for 2 h. After completion of the reaction, the solvent was removed and chloroform was added. The black residue was filtered through Celite® and the filtrate was concentrated. The residue obtained was purified by column chromatography (silica gel, 40-50% ethyl acetate in chloroform) to afford the title compound. Yield: 175 mg (72%); ¹H NMR (DMSO-d₆, 300 MHz): δ 8.39 (bs, 1H), 7.89 (s, 1H), 7.60 (bs, 1H), 7.49-7.46 (d, 4H), 7.15-7.03 (m, 3H), 6.95-6.87 (m, 2H), 3.62 (s, 3H), 2.88 (m, 2H), 1.96 (m, 2H), 1.19 (s, 6H); MS: m/z 441.2 (M+1).

### Example 269

### 4-(5-(4-(3-(2-Fluorophenyl)guanidino)phenyl)thiazol-2-yl)-2,2-dimethyl butanoic acid

The compound of example 269 was prepared analogous to the compound of example 7 by hydrolysis of the compound of example 268. Yield: 71%; ¹H NMR (DMSO-d₆, 300 MHz): δ 11.60 (bs, 1H), 9.78 (bs, 1H), 7.91 (s, 1H), 7.49 (dd, 4H), 7.18-7.05 (m, 3H), 6.97 (m, 1H), 5.58 (bs, 2H), 2.91 (m, 2H), 1.93 (m, 2H), 1.17 (s, 6H); MS: m/z 427.2 (M+1).

### Example 270

### Methyl 4-(5-(4-(3-(2-fluorophenyl)-2-methylguanidino)phenyl)thiazol-2-yl)-2,2-dimethylbutanoate

The compound of example 270 was prepared analogous to the compound of example 268 by reaction of the compound of example 266 with methanamine. Yield: 91%; ¹H NMR (DMSO-d₆, 300 MHz): δ 7.95 (s, 1H), 7.86 (s, 1H), 7.42-7.39 (d, 2H), 7.25-7.22 (d, 2H), 7.01-6.93 (m, 2H), 6.91-6.82 (m, 2H), 5.89 (s, 1H), 3.61 (s, 3H), 2.89 (m, 2H), 2.72 (s, 3H), 1.95 (m, 2H), 1.19 (s, 6H); MS: m/z 455.2 (M+1).

### Example 271

### 4-(5-(4-(3-(2-Fluorophenyl)-2-methylguanidino)phenyl)thiazol-2-yl)-2,2-dimethyl butanoic acid

The compound of example 271 was prepared analogous to the compound of example 7 by hydrolysis of the compound of example 270. Yield: 47%; ¹H NMR (DMSO-d₆, 300 MHz): δ 12.07 (bs, 1H), 7.86 (s, 1H), 7.42-7.39 (d, 2H), 7.20-7.17 (d, 2H), 7.05-6.94 (m, 2H), 6.90-6.81 (m, 2H), 5.95 (bs, 1H), 3.17 (s, 1H), 2.89 (m, 2H), 2.72 (s, 3H), 1.92 (m, 2H), 1.16 (s, 6H); MS: m/z 455.2 (M+1).

### Example 272

### Methyl 4-(5-(4-(2-cyano-3-(2-fluorophenyl)guanidino)phenyl)thiazol-2-yl)-2,2-dimethylbutanoate

The compound of example 272 was prepared analogous to the compound of example 268 by reaction of the compound of example 266 with cyanamide. Yield: 73%; ¹H NMR (DMSO-d₆, 300 MHz): δ 9.56 (s, 1H), 9.43 (s, 1H), 8.00 (s, 1H), 7.62-7.59 (d, 2H), 7.37-7.35 (d, 2H), 7.33-7.25 (m, 2H), 7.23-7.19 (m, 1H), 6.21 (s, 1H), 3.62 (s, 3H), 2.90 (m, 2H), 1.99 (m, 2H), 1.20 (s, 6H); MS: m/z 466.2 (M+1).

### Example 273

### 4-(5-(4-(2-Cyano-3-(2-fluorophenyl)guanidino)phenyl)thiazol-2-yl)-2,2-dimethyl butanoic acid

The compound of example 273 was prepared analogous to the compound of example 7 by hydrolysis of the compound of example 272. Yield: 91%; ¹H NMR (DMSO-d₆, 300 MHz): δ 12.30 (bs, 1H), 9.63 (s, 1H), 9.48 (s, 1H), 8.00 (s, 1H), 7.62-7.59 (d, 2H), 7.37-7.35 (d, 2H), 7.29-7.26 (m, 3H), 7.23-7.19 (m, 1H), 2.90 (m, 2H), 1.94 (m, 2H), 1.17 (s, 6H); MS: m/z 452.2 (M+1).

### Example 274

### Methyl 5-(2-(4-nitrobenzoyl)hydrazinyl)-5-oxopentanoate

To a cooled solution of commercially available 4-nitro benzohydrazide (10 g, 55.2 mmol) in dichloromethane (300 mL) was added methyl 5-chloro-5-oxopentanoate (10.9 g, 66.2 mmol). After 15 min of stirring at room temperature, the reaction mixture was diluted with dichloromethane and washed with water and brine, dried over sodium sulphate and concentrated. The crude material obtained was directly used for the next step without purification.

### Example 275

### Methyl 4-(5-(4-nitrophenyl)-1,3,4-thiadiazol-2-yl)butanoate

To a solution of the compound of example 274 (1.7 g, 5.5 mmol) in dioxane (35 mL) was added Lawesson's reagent (2.2 g, 5.5 mmol) and the reaction mixture was heated at 80° C. for 2-3 h. After completion of reaction, dioxane was removed and the material obtained was dissolved in water. The solution was made basic by adding aqueous sodium bicarbonate and extracted with ethyl acetate. The ethyl acetate extract was washed with water and brine, dried over sodium sulphate and concentated to obtain a crude residue, which was purified by column chromatography (silica gel, 30% ethyl acetate in petroleum ether). Yield: 83%; ¹H NMR (DMSO-d₆, 300 MHz): δ 8.39 (d, 2H), 8.25 (d, 2H), 3.60 (s, 3H), 3.24 (t, 2H), 2.48 (t, 2H), 2.07 (m, 2H); MS: m/z 308 (M+1).

### Example 276

### Methyl 4-(5-(4-aminophenyl)-1,3,4-thiadiazol-2-yl)butanoate

The compound of example 276 was prepared analogous to the compound of example 5 by reduction of the compound of example 275. Yield: 74%; ¹H NMR (DMSO-d₆, 300 MHz): δ 7.59 (d, 2H), 6.64 (d, 2H), 5.81 (s, 2H), 3.59 (s, 3H), 3.09 (t, 2H), 2.46 (t, 2H), 2.02 (m, 2H); MS: m/z 278 (M+1).

### Example 277

### Methyl 4-(5-(4-(3-(3-(trifluoromethyl)phenyl)ureido)phenyl)-1,3,4-thiadiazol-2-yl)butanoate

The compound of example 277 was prepared analogous to the compound of example by reaction of the compound of example 276 with 1-isocyanato-3-(trifluoromethyl)benzene. Yield: 81%; ¹H NMR (DMSO-d₆, 300 MHz): δ 9.18 (s, 1H), 9.16 (s, 1H), 8.03 (s, 1H), 7.89 (d, 2H), 7.65 (d, 2H), 7.58 (m, 2H), 7.35 (d, 1H), 3.60 (s, 3H), 3.16 (t, 2H), 2.46 (m, 2H), 2.04 (m, 2H); MS: m/z 465 (M+1).

### Example 278

### 4-(5-(4-(3-(3-(Trifluoromethyl)phenyl)ureido)phenyl)-1,3,4-thiadiazol-2-yl)butanoic acid

The compound of example 273 was prepared analogous to the compound of example 7 by hydrolysis of the compound of example 272. Yield: 89%; ¹H NMR (DMSO-d₆, 300 MHz): δ 12.14 (s, 1H), 9.18 (s, 1H), 9.16 (s, 1H), 8.03 (s, 1H), 7.89 (d, 2H), 7.62 (d, 2H), 7.59 (d, 1H), 7.53 (t, 1H), 7.35 (d, 1H), 3.16 (t, 2H), 2.42 (m, 2H), 2.03 (m, 2H); MS: m/z 449 (M−1).

### Example 279

### Methyl 4-(5-(4-(3-(2-chlorophenyl)ureido)phenyl)-1,3,4-thiadiazol-2-yl)butanoate

The compound of example 279 was prepared analogous to the compound of example 6 by reaction of the compound of example 276 with 2-chloro-1-isocyanatobenzene.

Yield: 80%; ¹H NMR (DMSO-d₆, 300 MHz): δ 9.75 (s, 1H), 8.41 (s, 1H), 8.17 (d, 1H), 7.90 (d, 2H), 7.65 (d, 2H), 7.49 (m, 1H), 7.34 (t, 1H), 7.08 (t, 1H), 3.60 (s, 3H), 3.16 (t, 2H), 2.46 (m, 2H), 2.06 (m, 2H); MS: m/z 431 (M+1).

### Example 280

### 4-(5-(4-(3-(2-Chlorophenyl)ureido)phenyl)-1,3,4-thiadiazol-2-yl)butanoic acid

The compound of example 280 was prepared analogous to the compound of example 7 by hydrolysis of the compound of example 279. Yield: 77%; ¹H NMR (DMSO-d₆, 300 MHz): δ 12.17 (s, 1H), 9.75 (s, 1H), 8.50 (s, 1H), 7.90 (s, 1H), 7.80 (d, 2H), 7.62 (d, 2H), 7.49 (d, 1H), 7.35 (t, 1H), 7.08 (t, 1H), 3.22 (t, 2H), 2.39 (m, 2H), 2.03 (m, 2H); MS: m/z 415 (M−1).

### Example 281

### Methyl 4-(5-(4-(3-(p-tolyl)ureido)phenyl)-1,3,4-thiadiazol-2-yl)butanoate

The compound of example 281 was prepared analogous to the compound of example 6 by reaction of the compound of example 276 with 1-isocyanato-4-methylbenzene.

Yield: 84%; ¹H NMR (DMSO-d₆, 300 MHz): δ 8.99 (s, 1H), 8.67 (s, 1H), 7.87 (d, 2H), 7.62 (d, 2H), 7.36 (d, 2H), 7.11 (m, 2H), 3.60 (s, 3H), 3.16 (t, 2H), 2.46 (m, 2H), 2.24 (s, 3H), 2.06 (m, 2H); MS: m/z 411 (M+1).

### Example 282

### 4-(5-(4-(3-(p-Tolyl)ureido)phenyl)-1,3,4-thiadiazol-2-yl)butanoic acid

The compound of example 282 was prepared analogous to the compound of example 7 by hydrolysis of the compound of example 281. Yield: 94%; ¹H NMR (DMSO-d₆, 300 MHz): δ 12.18 (s, 1H), 8.99 (s, 1H), 8.67 (s, 1H), 7.87 (d, 2H), 7.62 (d, 2H), 7.36 (d, 2H), 7.11 (m, 2H), 3.26 (t, 2H), 2.39 (m, 2H), 2.25 (s, 3H), 2.02 (m, 2H); MS: m/z 397 (M+1).

### Example 283

### Methyl 4-(5-(4-(3-(2,4-difluorophenyl)ureido)phenyl)-1,3,4-thiadiazol-2-yl)butanoate

The compound of example 283 was prepared analogous to the compound of example 6 by reaction of the compound of example 276 with 2,4-difluoro-1-isocyanatobenzene.

Yield: 84%; ¹H NMR (DMSO-d₆, 300 MHz): δ 9.36 (s, 1H), 8.61 (s, 1H), 8.20 (m, 1H), 7.89 (d, 2H), 7.63 (d, 2H), 7.37 (m, 1H), 7.10 (m, 1H), 3.60 (s, 3H), 3.16 (t, 2H), 2.49 (m, 2H), 2.06 (m, 2H); MS: m/z 431 (M−1).

### Example 284

### 4-(5-(4-(3-(2,4-Difluorophenyl)ureido)phenyl)-1,3,4-thiadiazol-2-yl)butanoic acid

The compound of example 284 was prepared analogous to the compound of example 7 by hydrolysis of the compound of example 283. Yield: 90%; ¹H NMR (DMSO-d₆, 300 MHz): δ 12.2 8 (s, 1H), 9.39 (s, 1H), 8.62 (s, 1H), 8.23 (m, 1H), 7.89 (d, 2H), 7.63 (d, 2H), 7.37 (d, 1H), 7.10 (m, 1H), 3.20 (t, 2H), 2.39 (m, 2H), 2.02 (m, 2H); MS: m/z 419 (M+1).

### Example 285

### Methyl 4-(5-(4-(3-(4-chloro-2-phenoxyphenyl)ureido)phenyl)-1,3,4-thiadiazol-2-yl)butanoate

The compound of example 285 was prepared analogous to the compound of example 6 by reaction of the compound of example 276 with 4-chloro-1-isocyanato-2-phenoxybenzene. Yield: 82%; ¹H NMR (DMSO-d₆, 300 MHz): δ 9.69 (s, 1H), 8.77 (s, 1H), 8.40 (m, 1H), 7.89 (d, 2H), 7.62 (d, 2H), 7.47 (m, 2H), 7.29 (t, 1H), 7.11 (d, 2H), 7.00 (dd, 1H), 6.85 (d, 1H), 3.60 (s, 3H), 3.16 (t, 2H), 2.46 (m, 2H), 2.06 (m, 2H); MS: m/z 521 (M−1).

### Example 286

### 4-(5-(4-(3-(4-Chloro-2-phenoxyphenyl)ureido)phenyl)-1,3,4-thiadiazol-2-yl)butanoic acid

The compound of example 286 was prepared analogous to the compound of example 7 by hydrolysis of the compound of example 285. Yield: 86%; ¹H NMR (DMSO-d₆, 300 MHz): δ 12.30 (s, 1H), 9.70 (s, 1H), 8.77 (s, 1H), 8.40 (d, 1H), 7.89 (d, 2H), 7.62 (d, 2H), 7.47 (t, 2H), 7.23 (t, 1H), 7.11 (d, 2H), 7.03 (m, 1H), 6.85 (d, 1H), 3.20 (t, 2H), 2.39 (m, 2H), 2.02 (m, 2H); MS: m/z 509 (M+1).

### Example 287

### Methyl 4-(5-(4-(4-(tert-butyl)benzamido)phenyl)-1)-1,3,4-thiadiazol-2-yl)butanoate

The compound of example 287 was prepared analogous to the compound of example 14 by reaction of the compound of example 276 with 4-t-butyl benzoyl chloride. Yield: 77%; ¹H NMR (DMSO-d₆, 300 MHz): δ 10.46 (s, 1H), 7.99 (m, 4H), 7.92 (d, 2H), 7.58 (d, 2H), 3.60 (s, 3H), 3.17 (t, 2H), 2.47 (m, 2H), 2.07 (m, 2H) 1.33 (s, 9H); MS: m/z 438 (M+1).

### Example 288

### 4-(5-(4-(4-(tert-Butyl)benzamido)phenyl)-1,3,4-thiadiazol-2-yl)butanoic acid

The compound of example 286 was prepared analogous to the compound of example 15 by hydrolysis of the compound of example 287. Yield: 77%; ¹H NMR (DMSO-d₆, 300 MHz): δ 12.21 (s, 1H), 10.46 (s, 1H), 7.99 (m, 4H), 7.92 (d, 2H), 7.58 (d, 2H), 3.17 (t, 2H), 2.38 (m, 2H), 2.03 (m, 2H) 1.33 (s, 9H); MS: m/z 424 (M+1).

### Example 289

### Methyl 4-(5-(4-([1,1′-biphenyl]-4-ylcarboxamido)phenyl)-1,3,4-thiadiazol-2-yl)butanoate

The compound of example 289 was prepared analogous to the compound of example 14 by reaction of the compound of example 276 with 4-phenyl benzoyl chloride. Yield: 91%; ¹H NMR (DMSO-d₆, 300 MHz): δ 10.59 (s, 1H), 8.83 (d, 2H), 8.39 (t, 1H), 8.11 (d, 1H), 8.03 (m, 2H), 7.88 (m, 3H), 7.81 (m, 2H), 7.52 (m, 1H), 7.52 (m, 1H), 3.61 (s, 3H), 3.16 (t, 2H), 2.49 (m, 2H), 2.02 (m, 2H); MS: m/z 458 (M+1).

### Example 290

4-(5-(4-([1,1′-Biphenyl]-4-ylcarboxamido)phenyl)-1,3,4-thiadiazol-2-yl)butanoic acid

The compound of example 289 was prepared analogous to the compound of example 15 by hydrolysis of the compound of example 288. Yield: 55%; ¹H NMR (DMSO-d₆, 300 MHz): δ 10.57 (s, 1H), 8.08 (d, 2H), 7.97 (t, 2H), 7.83 (m, 3H), 7.51 (m, 6H), 3.15 (t, 2H), 2.38 (m, 2H), 2.01 (m, 2H); MS: m/z 442 (M−1).

### Example 291

### Methyl 4-(5-(4-(4-trifluoromethoxy)benzamido)phenyl)-1,3,4-thiadiazol-2-yl)butanoate

The compound of example 291 was prepared analogous to the compound of example 14 by reaction of the compound of example 276 with 4-trifluoromethoxy benzoyl chloride. Yield: 91%; ¹H NMR (DMSO-d₆, 300 MHz): δ 10.64 (s, 1H), 8.12 (d, 2H), 7.96 (m, 4H), 7.57 (d, 2H), 3.60 (s, 3H), 3.18 (t, 2H), 2.49 (m, 2H), 2.07 (m, 2H); MS: m/z 466 (M+1).

### Example 292

4-(5-(4-(4-(Trifluoromethoxy)benzamido)phenyl)-1,3,4-thiadiazol-2-yl)butanoic acid

The compound of example 292 was prepared analogous to the compound of example 15 by hydrolysis of the compound of example 291. Yield: 89%; ¹H NMR (DMSO-d₆, 300 MHz): δ 10.63 (s, 1H), 8.10 (d, 2H), 7.94 (m, 4H), 7.51 (d, 2H), 3.16 (t, 2H), 2.38 (m, 2H), 2.01 (m, 2H); MS: m/z 450 (M−1).

### Example 293

### Methyl 4-(5-(4-nitrophenyl)-1,3,4-oxadiazol-2-yl)butanoate

To a solution of the compound of example 274 (6.2 g, 20.05 mmol) and phosphorus oxychloride (33.7 g, 220 mmol) in dry acetonitrile (150 mL) was heated at reflux temperature for 2-3 h. After completion of reaction, the solvent was removed and the material obtained was taken in ice water. The solution was made basic by addition of sodium bicarbonate and was then extracted with ethyl acetate. The ethyl acetate extract was washed with water and brine, dried over sodium sulphate and concentrated. The crude material obtained was purified by column chromatography (silica gel, 30% ethyl acetate in petroleum ether). Yield: 51%; ¹H NMR (DMSO-d₆, 300 MHz): δ 8.41 (d, 2H), 8.26 (d, 2H), 3.71 (s, 3H), 3.10 (t, 2H), 2.69 (t, 2H), 2.29 (m, 2H); MS: m/z 292 (M+1).

### Example 294

### Methyl 4-(5-(4-aminophenyl)-1,3,4-oxadiazol-2-yl)butanoate

The compound of example 294 was prepared analogous to the compound of example 5 by reduction of the compound of example 293. Yield: 84%; ¹H NMR (DMSO-d₆, 300 MHz): δ 7.62 (d, 2H), 6.67 (d, 2H), 5.88 (s, 2H), 3.59 (s, 3H), 2.92 (t, 2H), 2.46 (t, 2H), 2.03 (m, 2H); MS: m/z 262 (M+1).

### Example 295

### Methyl 4-(5-(4-(3-(2-chlorophenyl)ureido)phenyl)-1,3,4-oxadiazol-2-yl)butanoate

The compound of example 285 was prepared analogous to the compound of example 6 by reaction of the compound of example 294 with 2-chloro-1-isocyanatobenzene.

Yield: 79%; ¹H NMR (DMSO-d₆, 300 MHz): δ 9.79 (s, 1H), 8.16 (d, 1H), 7.92 (d, 2H), 7.69 (d, 2H), 7.49 (dd, 1H), 7.34 (m, 1H), 7.09 (m, 1H), 3.59 (s, 3H), 2.98 (t, 2H), 2.49 (m, 2H), 2.03 (m, 2H); MS: m/z 415 (M+1).

### Example 296

### 4-(5-(4-(3-(2-Chlorophenyl)ureido)phenyl)-1,3,4-oxadiazol-2-yl)butanoic acid

The compound of example 296 was prepared analogous to the compound of example 7 by hydrolysis of the compound of example 295. Yield: 83%; ¹H NMR (DMSO-d₆, 300 MHz): δ 12.24 (s, 1H), 9.79 (s, 1H), 8.43 (s, 1H), 8.17 (dd, 1H), 7.93 (d, 2H), 7.93 (d, 2H), 7.50 (dd, 1H), 7.35 (m, 1H), 7.09 (m, 1H), 2.98 (m, 2H), 2.42 (t, 2H), 2.03 (m, 2H); MS: m/z 401 (M+1).

### Example 297

### Methyl 4-(5-(4-(3-(m-tolyl)ureido)phenyl)-1,3,4-oxadiazol-2-yl)butanoate

The compound of example 297 was prepared analogous to the compound of example 6 by reaction of the compound of example 294 with 1-isocyanato-3-methylbenzene.

Yield: 89%; ¹H NMR (DMSO-d₆, 300 MHz): δ 9.07 (s, 1H), 7.91 (d, 2H), 7.68 (d, 2H), 7.32 (s, 1H), 7.26 (d, 1H), 7.20 (m, 2H), 6.83 (d, 1H), 3.60 (s, 3H), 2.98 (t, 2H), 2.49 (m, 2H), 2.29 (s, 3H), 2.04 (m, 2H); MS: m/z 395 (M+1).

### Example 298

### 4-(5-(4-(3-(m-Tolyl)ureido)phenyl)-1,3,4-oxadiazol-2-yl)butanoic acid

The compound of example 298 was prepared analogous to the compound of example 7 by hydrolysis of the compound of example 297. Yield: 91%; ¹H NMR (DMSO-d₆, 300 MHz): δ 12.18 (s, 1H), 9.07 (s, 1H), 8.75 (s, 1H), 7.91 (d, 2H), 7.67 (d, 2H), 7.32 (s, 1H), 7.26 (d, 1H), 7.20 (t, 1H), 6.83 (d, 1H), 2.93 (t, 2H), 2.42 (t, 2H), 2.29 (s, 3H), 2.03 (m, 2H); MS: m/z 381 (M+1).

### Example 299

### Methyl 4-(5-(4-(3-(2,4-difluorophenyl)ureido)phenyl)-1,3,4-oxadiazol-2-yl)butanoate

The compound of example 299 was prepared analogous to the compound of example 6 by reaction of the compound of example 294 with 2,4-difluoro-1-isocyanatobenzene.

Yield: 83%; ¹H NMR (DMSO-d₆, 300 MHz): δ 9.41 (s, 1H), 8.96 (s, 1H), 8.11 (m, 1H), 7.92 (d, 2H), 7.67 (d, 2H), 7.38 (m, 1H), 7.10 (m, 1H), 3.59 (s, 3H), 2.98 (t, 2H), 2.47 (m, 2H), 2.07 (m, 2H); MS: m/z 417 (M+1).

### Example 300

### 4-(5-(4-(3-(2,4-Difluorophenyl)ureido)phenyl)-1,3,4-oxadiazol-2-yl)butanoic acid

The compound of example 300 was prepared analogous to the compound of example 7 by hydrolysis of the compound of example 299. Yield: 90%; ¹H NMR (DMSO-d₆, 300 MHz): δ 12.18 (s, 1H), 9.41 (s, 1H), 8.63 (s, 1H), 8.09 (m, 1H), 7.92 (d, 2H), 7.67 (d, 2H), 7.34 (dd, 1H), 7.07 (m, 1H), 2.97 (m, 2H), 2.41 (t, 2H), 2.00 (m, 2H); MS: m/z 403 (M+1).

### Example 301

### Methyl 4-(5-(4-(3-(3-(trifluoromethyl)phenyl)ureido)phenyl)-1,3,4-oxadiazol-2-yl)butanoate

The compound of example 301 was prepared analogous to the compound of example 6 by reaction of the compound of example 294 with 1-isocyanato-3-trifluoromethyl benzene.

### Example 302

### 4-(5-(4-(3-(3-(Trifluoromethyl)phenyl)ureido)phenyl)-1,3,4-oxadiazol-2-yl)butanoic acid

The compound of example 302 was prepared analogous to the compound of example 7 by hydrolysis of the compound of example 301. Yield: 94%; ¹H NMR (DMSO-d₆, 300 MHz): δ 12.18 (s, 1H), 9.22 (s, 1H), 9.18 (s, 1H), 8.04 (s, 1H), 7.92 (d, 2H), 7.70 (d, 2H), 7.61 (m, 2H), 7.35 (d, 1H), 2.98 (m, 2H), 2.42 (t, 2H), 2.00 (m, 2H); MS: m/z 435 (M+1).

### Example 303

### (E)-3-(dimethylamino)-1-(4-nitrophenyl)prop-2-en-1-one

A mixture of commercially available 1-(4-nitrophenyl)ethanone (6 g, 36.3 mmol) and DMF-DMA (8.99 ml, 67.1 mmol) was refluxed for 17 h. After completion of reaction, reaction mixture was cooled and solid obtained was recrystallized from diethyl ether.

Yield: 82%; ¹H NMR (DMSO-d₆, 300 MHz): δ 8.28 (d, 2H), 8.04 (d, 2H), 7.90 (d, 1H), 5.71 (d, 1H), 3.22 (s, 3H), 2.99 (s, 3H); MS: m/z 221 (M+1).

### Example 304

### t-Butyl 2-((1r,4r)-4-(ethoxycarbonyl)cyclohexyl)hydrazinecarboxylate

To a solution of ethyl 4-oxocyclohexanecarboxylate (8 g, 47.0 mmol) and t-butyl hydrazinecarboxylate (6.21 g, 47.0 mmol) in dichloromethane (540 mL), acetic acid (5.4 mL) and sodium triacetoxyhydroborate (30 g, 142 mmol) were added at 0° C. The reaction mixture was gradually cooled to room temperature and stirred for 7-8 h. The reaction mixture was poured into saturated aqueous sodium hydrogen carbonate solution and the mixture was extracted with ethyl acetate. The organic layer was washed with water and brine, dried over anhydrous sodium sulfate and concentrated to yield a crude material. The crude material was purified by column chromatography (silica gel, 30% ethyl acetate in petroleum ether). Yield: 97%; ¹H NMR (DMSO-d₆, 300 MHz): δ 6.04 (s, 1H), 4.16 (q, 2H), 4.08 (s, 1H), 2.81 (m, 1H), 2.25 (m, 1H), 2.04 (m, 4H), 1.47 (m, 11H), 1.28 (t, 3H), 1.42 (m, 2H); MS: m/z 287 (M+1).

### Example 305

### (1r,4r)-Ethyl 4-hydrazinylcyclohexanecarboxylate

The compound of example 304 (15 g, 52.4 mmol) was dissolved in dioxane (165 mL) and to the reaction mixture, 5 mL of HCl in dioxane (50 mL) was added and the reaction mixture was stirred for 15-16 h at 40-45° C. After cooling, diethyl ether was added and the solid obtained was filtered and dried. Yield: 97%; ¹H NMR (DMSO-d₆, 300 MHz): δ 4.08 (q, 2H), 2.86 (m, 1H), 2.27 (m, 1H), 2.15 (m, 4H), 1.40 (m, 4H), 1.21 (t, 3H); MS: m/z 187 (M+1).

### Example 306

### Ethyl 4-(3-(4-nitrophenyl)-1H-pyrazol-1-yl)cyclohexanecarboxylate

To a solution of the compound of example 303 (300 mg, 1.362 mmol) and compound of example 305 (507 mg, 2.72 mmol) in ethanol (10 mL) was heated at 65° C. for 1 h. After completion of the reaction, the reaction mixture was cooled and crystallised solid material was filtered and dried. Yield: 53%; ¹H NMR (DMSO-d₆, 300 MHz): δ 8.35 (d, 2H), 7.74 (d, 2H), 7.58 (s, 1H), 6.50 (s, 1H), 4.18 (m, 1H), 4.07 (q, 2H), 2.39 (m, 1H), 1.98 (m, 6H), 1.50 (m, 2H), 1.18 (t, 3H); MS: m/z 344 (M+1).

### Example 307

### Ethyl 4-(3-(4-nitrophenyl)-1H-pyrazol-1-yl)cyclohexanecarboxylate

The compound of example 307 was prepared analogous to the compound of example 5 by reduction of the compound of example 306. Yield: 88%; ¹H NMR (DMSO-d₆, 300 MHz): δ 7.40 (d, 1H), 7.04 (d, 2H), 6.65 (d, 2H), 6.10 (d, 1H), 5.36 (s, 2H), 4.16 (m, 3H), 2.63 (m, 1H), 2.15 (m, 2H), 1.99 (m, 2H), 1.71 (m, 2H), 1.57 (m, 2H), 1.24 (t, 3H); MS: m/z 314 (M+1).

### Example 308

### Ethyl 4-(3-(4-(3-(2-chlorophenyl)ureido)phenyl)-1H-pyrazol-1-yl)cyclohexane carboxylate

The compound of example 308 was prepared analogous to the compound of example 6 by reaction of the compound of example 307 with 2-chloro-1-isocyanato benzene.

Yield: 83%; ¹H NMR (DMSO-d₆, 300 MHz): δ 9.60 (s, 1H), 8.38 (s, 1H), 8.18 (d, 1H), 7.61 (d, 2H), 7.48 (d, 2H), 7.36 (m, 3H), 7.07 (m, 1H), 6.24 (d, 1H), 4.16 (q, 2H), 2.64 (m, 1H), 2.15 (m, 2H), 1.99 (m, 2H), 1.74 (m, 2H), 1.59 (m, 3H), 1.24 (t, 3H); MS: m/z 467 (M+1).

### Example 309

### 4-(3-(4-(3-(2-Chlorophenyl)ureido)phenyl)-1H-pyrazol-1-yl)cyclohexane carboxylic acid

The compound of example 309 was prepared analogous to the compound of example 7 by hydrolysis of the compound of example 308. Yield: 89%; ¹H NMR (DMSO-d₆, 300 MHz): δ 12.25 (s, 1H), 9.65 (s, 1H), 8.42 (s, 1H), 8.18 (d, 1H), 7.61 (d, 2H), 7.48 (d, 2H), 7.35 (m, 3H), 7.07 (m, 1H), 6.24 (d, 1H), 4.14 (m, 1H); 2.72 (m, 1H), 2.26 (m, 2H), 2.02 (m, 2H), 1.73 (m, 2H), 1.50 (m, 2H); MS: m/z 439 (M+1).

### Example 310

### Ethyl 4-(3-(4-(3-(2-fluorophenyl)ureido)phenyl)-1H-pyrazol-1-yl)cyclohexane carboxylate

The compound of example 310 was prepared analogous to the compound of example 6 by reaction of the compound of example 307 with 2-fluoro-1-isocyanato benzene.

Yield: 83%; ¹H NMR (DMSO-d₆, 300 MHz): δ 9.26 (s, 1H), 8.62 (s, 1H), 8.18 (d, 1H), 7.59 (d, 2H), 7.47 (s, 1H), 7.35 (d, 2H), 7.28 (m, 1H), 7.18 (t, 1H), 7.05 (m, 1H), 6.24 (d, 1H), 4.16 (q, 2H), 2.64 (m, 1H), 2.15 (m, 2H), 1.99 (m, 2H), 1.74 (m, 2H), 1.59 (m, 3H), 1.23 (t, 3H); MS: m/z 449 (M−1).

### Example 311

### 4-(3-(4-(3-(2-Fluorophenyl)ureido)phenyl)-1H-pyrazol-1-yl)cyclohexane carboxylic acid

The compound of example 311 was prepared analogous to the compound of example 7 by hydrolysis of the compound of example 310. Yield: 92%; ¹H NMR (DMSO-d₆, 300 MHz): δ 12.23 (s, 1H), 9.26 (s, 1H), 8.62 (s, 1H), 8.19 (t, 1H), 7.60 (d, 2H), 7.49 (d, 1H), 7.35 (m, 2H), 7.28 (m, 1H), 7.18 (t, 1H), 7.06 (m, 1H), 6.26 (m, 1H), 4.14 (m, 1H), 2.56 (m, 1H), 2.14 (m, 2H), 2.02 (m, 2H), 1.73 (m, 2H), 1.55 (m, 2H); MS: m/z 423 (M+1).

### Example 312

### Ethyl 4-(3-(4-(3-(2,4-difluorophenyl)ureido)phenyl)-1H-pyrazol-1-yl)cyclohexane carboxylate

The compound of example 312 was prepared analogous to the compound of example 6 by reaction of the compound of example 307 with 2,4-difluoro-1-isocyanato benzene.

Yield: 90%; ¹H NMR (DMSO-d₆, 300 MHz): δ 9.20 (s, 1H), 8.57 (s, 1H), 8.13 (m, 1H), 7.59 (d, 2H), 7.47 (s, 1H), 7.34 (d, 3H), 7.09 (m, 1H), 6.24 (d, 1H), 4.16 (q, 2H), 2.63 (m, 1H), 2.15 (m, 2H), 2.02 (m, 2H), 1.74 (m, 2H), 1.58 (m, 3H), 1.23 (t, 3H); MS: m/z 469 (M+1).

### Example 313

### 4-(3-(4-(3-(2,4-Difluorophenyl)ureido)phenyl)-1H-pyrazol-1-yl)cyclohexane carboxylic acid

The compound of example 313 was prepared analogous to the compound of example 7 by hydrolysis of the compound of example 312. Yield: 91%; ¹H NMR (DMSO-d₆, 300 MHz): δ 12.50 (s, 1H), 9.21 (s, 1H), 8.57 (s, 1H), 8.10 (m, 1H), 7.59 (d, 2H), 7.45 (d, 1H), 7.34 (m, 3H), 7.06 (m, 1H), 6.24 (m, 1H), 4.14 (m, 1H), 2.55 (m, 1H), 2.14 (m, 2H), 2.01 (m, 2H), 1.72 (m, 2H), 1.54 (m, 2H); MS: m/z 441 (M+1).

### Example 314

### Ethyl 4-(3-(4-(3-(3-(trifluoromethyl)phenyl)ureido)phenyl)-1H-pyrazol-1-yl)cyclohexanecarboxylate

The compound of example 314 was prepared analogous to the compound of example 6 by reaction of the compound of example 307 with 1-isocyanato-3-trifluoromethyl benzene. Yield: 81%; ¹H NMR (DMSO-d₆, 300 MHz): δ 9.12 (s, 1H), 9.00 (s, 1H), 8.03 (s, 1H), 7.61 (d, 2H), 7.55 (m, 2H), 7.53 (d, 1H), 7.35 (d, 3H), 6.25 (s, 1H), 4.16 (q, 2H), 2.64 (m, 1H), 2.12 (m, 2H), 1.99 (m, 2H), 1.94 (m, 2H), 1.49 (m, 3H), 1.24 (t, 3H); MS: m/z 501 (M+1).

### Example 315

### 4-(3-(4-(3-(3-(Trifluoromethyl)phenyl)ureido)phenyl)-1H-pyrazol-1-yl)cyclohexanecarboxylic acid

The compound of example 315 was prepared analogous to the compound of example 7 by hydrolysis of the compound of example 314. Yield: 90%; ¹H NMR (DMSO-d₆, 300 MHz): δ 12.25 (s, 1H), 9.12 (s, 1H), 9.00 (s, 1H), 8.03 (m, 1H), 7.61 (d, 3H), 7.55 (m, 1H), 7.46 (d, 1H), 7.35 (d 3H), 6.24 (m, 1H), 4.14 (m, 1H), 2.56 (m, 1H), 2.15 (m, 2H), 2.02 (m, 2H), 1.73 (m, 2H), 1.54 (m, 2H); MS: m/z 473 (M+1).

### Example 316

### Ethyl 4-(3-(4-(3-(m-tolyl)ureido)phenyl)-1H-pyrazol-1-yl)cyclohexane carboxylate

The compound of example 316 was prepared analogous to the compound of example 6 by reaction of the compound of example 307 with 1-isocyanato-3-methyl benzene.

Yield: 95%; ¹H NMR (DMSO-d₆, 300 MHz): δ 8.84 (s, 1H), 8.65 (s, 1H), 7.59 (d, 2H), 7.46 (s, 1H), 7.33 (m, 3H), 7.26 (d, 1H), 7.195 (t, 1H), 6.81 (d, 1H), 6.23 (s, 1H), 4.16 (q, 2H), 2.64 (m, 1H), 2.28 (s, 3H), 2.16 (m, 2H), 1.99 (m, 2H), 1.74 (m, 2H), 1.58 (m, 3H), 1.23 (t, 3H); MS: m/z 447 (M+1).

### Example 317

### 4-(3-(4-(3-(m-Tolyl)ureido)phenyl)-1H-pyrazol-1-yl)cyclohexanecarboxylic acid

The compound of example 317 was prepared analogous to the compound of example 7 by hydrolysis of the compound of example 316. Yield: 92%; ¹H NMR (DMSO-d₆, 300 MHz): δ 12.24 (s, 1H), 8.85 (s, 1H), 8.65 (s, 1H), 7.59 (d, 2H), 7.48 (d, 1H), 7.33 (m, 3H), 7.26 (d, 1H), 7.16 (d, 1H), 6.81 (d, 1H), 6.25 (m, 1H), 4.14 (m, 1H), 2.56 (m, 1H), 2.28 (s, 3H), 2.15 (m, 2H), 2.02 (m, 2H), 1.73 (m, 2H), 1.54 (m, 2H); MS: m/z 419 (M+1).

### Example 318

### N′-hydroxy-4-nitrobenzimidamide

To a solution of commercially available 4-nitrobenzonitrile (15 g, 0.101 mol) in ethanol (100 mL), potassium carbonate (20.98 g, 0.152 mol) and hydroxylamine hydrochloride (10.56 g, 0.152 mol) were added. The reaction mixture was refluxed at 80° C. for 5 h. After completion of the reaction the solvent was removed and the crude obtained was dissolved in ethyl acetate. The ethyl acetate layer was washed with water and brine, dried over sodium sulfate and concentrated to yield a solid. The crude solid obtained was purified by column chromatography (silica gel, ethyl acetate in petroleum ether) and further crystallized from ethyl acetate in petroleum ether to afford the title compound. Yield: 12.4 g (68%); ¹H NMR (DMSO-d6, 300 MHz): δ 10.13 (s, 1H), 8.24 (d, 2H), 7.95 (d, 2H), 6.06 (s, 2H); MS: m/z 182 (M+1).

### Example 319

### Methyl 4-(3-(4-nitrophenyl)-1,2,4-oxadiazol-5-yl)butanoate

The compound of example 318 (2 g, 11.04 mmol) was taken in toluene (20 mL) and methyl 5-chloro-5-oxopentanoate (2.73 g, 16.56 mmol) was added dropwise. The reaction mixture was heated at 110° C. for 3-4 h. After completion of the reaction the reaction mixture was concentrated and the resulting mass was dissolved in ethyl acetate. The ethyl acetate layer was washed with water and brine, concentrated and dried to yield a crude residue, which was purified with column chromatography (silica gel, 30% ethyl acetate in petroleum ether) to afford the title compound. Yield: 2.83 g (88%); ¹H NMR (DMSO-d₆, 300 MHz): δ 8.42 (d, 2H), 8.28 (d, 2H), 3.60 (s, 3H), 3.12 (t, 2H), 2.36 (m, 2H), 2.12 (m, 2H); MS: m/z 313 (M+1).

### Example 320

### Methyl 4-(3-(4-aminophenyl)-1,2,4-oxadiazol-5-yl)butanoate

The compound of example 320 was prepared analogous to the compound of example 5 by reduction of the compound of example 319. Yield: 91%; ¹H NMR (DMSO-d₆, 300 MHz): δ 7.66 (d, 2H), 7.65 (d, 2H), 5.74 (s, 2H), 3.60 (s, 3H), 2.99 (t, 2H), 2.36 (m, 2H), MS: m/z 262 (M+1).

### Example 321

### Methyl 4-(3-(4-(3-(4-chloro-2-phenoxyphenyl)ureido)phenyl)-1,2,4-oxadiazol-5-yl)butanoate

The compound of example 321 was prepared analogous to the compound of example 6 by reaction of the compound of example 320 with 4-chloro-1-isocyanato-2-phenoxy benzene. Yield: 45% ¹H NMR (DMSO-d₆, 300 MHz): δ 8.42 (d, 1H), 8.11 (s, 1H), 8.00 (d, 2H), 7.85 (s, 1H), 7.64 (d, 2H), 7.44 (m, 2H), 7.20 (m, 1H), 7.10 (d, 2H), 7.00 (dd, 1H), 6.90 (d, 1H), 3.65 (s, 3H), 3.03 (t, 2H), 2.52 (t, 2H), 2.20 (t, 2H), MS: m/z 507 (M+1).

### Example 322

### 4-(3-(4-(3-(4-Chloro-2-phenoxyphenyl)ureido)phenyl)-1,2,4-oxadiazol-5-yl) butanoic acid

The compound of example 322 was prepared analogous to the compound of example 7 by hydrolysis of the compound of example 321. Yield: ¹H NMR (DMSO-d₆, 300 MHz): δ 12.22 (s, 1H), 9.69 (s, 1H), 8.77 (s, 1H), 8.40 (d, 1H), 8.95 (d, 2H), 7.64 (d, 2H), 7.45 (m, 2H), 7.20 (m, 1H), 7.11 (d, 2H), 7.01 (dd, 1H), 6.86 (d, 1H), 3.04 (t, 2H), 2.42 (t, 2H), 2.02 (t, 2H); MS: m/z 493 (M+1).

### Example 323

### Methyl 4-(3-(4-(3-(2,4-difluorophenyl)ureido)phenyl)-1,2,4-oxadiazol-5-yl)butanoate

The compound of example 323 was prepared analogous to the compound of example 6 by reaction of the compound of example 320 with 2,4-difluoro-1-isocyanatobenzene.

Yield: 26.40% ¹H NMR (DMSO-d₆, 300 MHz): δ 8.03 (m, 3H), 7.53 (d, 2H), 7.09 (s, 1H), 6.88 (m, 3H), 3.72 (s, 3H), 3.06 (t, 2H), 2.56 (t, 2H), 2.28 (m, 2H), MS: m/z 417 (M+1).

### Example 324

### 4-(3-(4-(3-(2,4-Difluorophenyl)ureido)phenyl)-1,2,4-oxadiazol-5-yl)butanoic acid

The compound of example 324 was prepared analogous to the compound of example 7 by hydrolysis of the compound of example 323. Yield: 78% ¹H NMR (DMSO-d₆, 300 HZ): δ 12.26 (s, 1H), 9.38 (s, 1H), 8.62 (s, 1H), 8.12 (m, 1H), 7.95 (d, 2H), 7.65 (d, 2H), 7.37 (m, 1H), 7.07 (m, 1H), 3.04 (t, 2H), 2.42 (t, 2H), 2.03 (m, 2H); MS: m/z 402 (M+1).

### Example 325

### Methyl 4-(3-(4-(3-(2-chlorophenyl)ureido)phenyl)-1,2,4-oxadiazol-5-yl)butanoate

The compound of example 325 was prepared analogous to the compound of example 6 by reaction of the compound of example 320 with 2-chloro-1-isocyanatobenzene.

Yield: 46%; ¹H NMR (DMSO-d₆, 300 MHz): δ 8.19 (d, 1H), 8.07 (d, 2H), 7.57 (d, 2H), 7.40 (d, 1H), 7.33 (m, 1H), 7.16 (s, 1H), 7.07 (m, 2H), 3.72 (s, 3H), 3.06 (t, 2H), 2.56 (t, 2H), 2.29 (m, 2H); MS: m/z 415 (M+1).

### Example 326

### 4-(3-(4-(3-(2-Chlorophenyl)ureido)phenyl)-1,2,4-oxadiazol-5-yl)butanoic acid

The compound of example 326 was prepared analogous to the compound of example 7 by hydrolysis of the compound of example 325. Yield: 86% ¹H NMR (DMSO-d₆, 300 MHz): δ 12.20 (s, 1H), 9.75 (s, 1H), 8.42 (s, 1H), 8.18 (dd, 1H), 7.96 (d, 2H), 7.67 (d, 2H), 7.50 (dd, 1H), 7.32 (m, 1H), 7.09 (m, 1H), 3.04 (t, 2H), 2.42 (t, 2H), 2.03 (m, 2H); MS: m/z 400 (M+1).

### Example 327

### Methyl 2,2-dimethyl-4-(3-(4-nitrophenyl)-1,2,4-oxadiazol-5-yl)butanoate

5-methoxy-4,4-dimethyl-5-oxopentanoic acid (1.82 g, 10.45 mmol) was taken in DCM (30 mL) and CDI (2.54 g, 15.67 mmol) was added at room temperature. This mixture was stirred for 1 h after which the compound of example 318 (3.41 g, 18.81 mmol) was added. The reaction mixture was further stirred for 8 h at room temperature. After 8 h, the reaction mixture was concentrated and toluene (25 mL) was added. This was further refluxed at 100° C. for 16 h. After complition of the reaction, the reaction mixture was cooled to room temperature, diluted with ethyl acetate, washed with water and brine and was dried using sodium sulphate. The organic layer was concentrated to yield a crude residue, which was purified by use of column chromatography (silica gel, 20% ethyl acetate in chloroform) to afford the title compound. Yield: 2.3 g (68.9%); ¹H NMR (DMSO-d₆, 300 MHz): δ 8.42 (d, 2H), 8.28 (d, 2H), 3.59 (s, 3H), 3.04 (t, 2H), 2.09 (t, 2H), 1.21 (s, 6H); MS: m/z 320 (M+1).

### Example 328

### Methyl 4-(3-(4-aminophenyl)-1,2,4-oxadiazol-5-yl)-2,2-dimethylbutanoate

The compound of example 328 was prepared analogous to the compound of example 5 by reduction of the compound of example 327. Yield: 78% ¹H NMR (DMSO-d₆, 300 MHz): δ 7.66 (d, 2H), 6.65 (d, 2H), 5.74 (s, 2H); 3.61 (s, 3H), 2.91 (t, 2H), 2.14 (t, 2H), 1.19 (s, 6H); MS: m/z 290 (M+1).

### Example 329

### Methyl 4-(3-(4-(3-(4-chloro-2-phenoxyphenyl)ureido)phenyl)-1,2,4-oxadiazol-5-yl)butanoate

The compound of example 329 was prepared analogous to the compound of example 6 by reaction of the compound of example 328 with 4-chloro-1-isocyanato-2-phenoxybenzene. Yield: 45.4% ¹H NMR (DMSO-d₆, 300 MHz): δ 9.68 (s, 1H), 8.76 (s, 1H), 8.40 (d, 1H), 7.94 (d, 2H), 7.63 (d, 2H), 7.47 (t, 2H), 7.20 (t, 1H), 7.11 (d, 2H), 7.03 (dd, 1H), 6.85 (d, 1H), 3.60 (s, 3H), 2.97 (t, 2H), 2.07 (t, 2H), 1.20 (s, 6H); MS: m/z 535 (M+1).

### Example 330

### 4-(3-(4-(3-(4-Chloro-2-phenoxyphenyl)ureido)phenyl-1)-1,2,4-oxadiazol-5-yl)butanoic acid

The compound of example 330 was prepared analogous to the compound of example 7 by hydrolysis of the compound of example 329. Yield: 48.6%, ¹H NMR (DMSO-d₆, 300 MHz): δ 9.80 (s, 1H), 8.86 (s, 1H), 8.39 (d, 1H), 7.94 (d, 2H), 7.64 (d, 2H), 7.46 (m, 2H), 7.22 (m, 1H), 7.11 (d, 2H), 7.03 (m, 1H), 6.85 (d, 2H), 2.35 (m, 2H), 2.01 (m, 2H), 1.16 (s, 6H); MS: m/z 520 (M+1).

### Example 331

### Methyl 4-(3-(4-(3-(2,4-difluorophenyl)ureido)phenyl)-1,2,4-oxadiazol-5-yl)butanoate

The compound of example 331 was prepared analogous to the compound of example 6 by reaction of the compound of example 328 with 2,4-difluoro-1-isocyanatobenzene.

Yield: 77% ¹H NMR (DMSO-d₆, 300 MHz): δ 9.35 (s, 1H), 8.60 (s, 1H), 8.09 (m, 1H), 7.94 (d, 2H), 7.64 (d, 2H), 7.33 (m, 1H), 7.07 (m, 1H), 3.60 (s, 3H), 2.97 (t, 2H), 2.07 (t, 2H), 1.20 (s, 6H), MS: m/z 445 (M+1).

### Example 332

### 4-(3-(4-(3-(2,4-Difluorophenyl)ureido)phenyl)-1,2,4-oxadiazol-5-yl)butanoic acid

The compound of example 332 was prepared analogous to the compound of example 7 by hydrolysis of the compound of example 331. Yield: 93% ¹H NMR (DMSO-d₆, 300 MHz): δ 12.37 (s, 1H), 9.48 (s, 1H) 8.67 (s, 1H), 8.11 (m, 1H), 7.94 (d, 2H), 7.64 (d, 2H), 7.37 (m, 1H), 7.09 (m, 1H), 2.97 (m, 2H), 2.02 (m, 2H), 1.17 (s, 6H); MS: m/z 430 (M+1).

### Example 333

### Methyl 4-(3-(4-(3-(2-chlorophenyl)ureido)phenyl)-1,2,4-oxadiazol-5-yl)butanoate

The compound of example 333 was prepared analogous to the compound of example 6 by reaction of the compound of example 328 with 2-chloro-1-isocyanatobenzene.

Yield: 51.3% ¹H NMR (DMSO-d₆, 300 MHz): δ 9.74 (s, 1H), 8.41 (s, 1H), 8.18 (d, 1H), 7.95 (d, 2H), 7.66 (d, 2H), 7.49 (d, 1H), 7.34 (m, 1H), 7.08 (m, 1H), 3.59 (s, 3H), 2.96 (m, 2H), 2.06 (m, 2H), 1.2 (s, 6H); MS: m/z 443 (M+1).

### Example 334

### 4-(3-(4-(3-(2-Chlorophenyl)ureido)phenyl)-1,2,4-oxadiazol-5-yl)butanoic acid

The compound of example 334 was prepared analogous to the compound of example 7 by hydrolysis of the compound of example 333. Yield: 51% ¹H NMR (DMSO-d₆, 300 MHz): δ 12.37 (s, 1H), 9.74 (s, 1H), 8.41 (s, 1H), 8.18 (d, 1H), 7.95 (d, 2H), 7.66 (d, 2H), 7.49 (d, 1H), 7.34 (m, 1H), 7.08 (m, 1H), 2.97 (m, 2H), 2.03 (m, 2H), 1.18 (s, 6H); MS: m/z 429 (M+1).

### Example 335

### Methyl 4-(3-(4-(4-fluorobenzamido)phenyl)-1,2,4-oxadiazol-5-yl)-2,2-dimethyl butanoate

The compound of example 335 was prepared analogous to the compound of example 14 by reaction of the compound of example 328 with 4-fluorobenzoyl chloride. Yield: 45.7% ¹H NMR (DMSO-d₆, 300 MHz): δ 10.59 (s, 1H), 8.92 (d, 2H), 8.10 (m, 5H), 7.42 (m, 1H), 3.60 (s, 3H), 2.97 (m, 2H), 2.08 (m, 2H), 1.21 (s, 6H); MS: m/z 412 (M+1).

### Example 336

### 4-(3-(4-(4-Fluorobenzamido)phenyl)-1,2,4-oxadiazol-5-yl)-2,2-dimethyl butanoic acid

The compound of example 336 was prepared analogous to the compound of example 7 by hydrolysis of the compound of example 335. Yield: 59.4% ¹H NMR (DMSO-d₆, 300 MHz): δ 12.37 (s, 1H), 10.54 (s, 1H), 8.09 (m, 2H), 8.03 (m, 4H), 7.43 (m, 2H), 2.98 (m, 2H), 2.04 (m, 2H), 1.18 (s, 6H); MS: m/z 417 (M+1).

### Example 337

### Methyl 4-(3-(4-([1,1′-biphenyl]-4-ylcarboxamido)phenyl)-1,2,4-oxadiazol-5-yl)-2,2-dimethyl butanoate

The compound of example 337 was prepared analogous to the compound of example 14 by reaction of the compound of example 328 with 4-phenyl benzoyl chloride. Yield: 92%; ¹H NMR (DMSO-d₆, 300 MHz): δ 10.61 (s, 1H), 8.90 (d, 2H), 8.53 (m, 1H), 8.11 (d, 1H), 8.02 (m, 2H), 7.88 (d, 1H), 7.82 (m, 2H), 7.55 (m, 2H), 7.46 (m, 2H), 3.69 (s, 3H), 2.99 (m, 2H), 2.09 (m, 2H), 1.21 (s, 6H); MS: m/z 470 (M+1).

### Example 338

### 4-(3-(4-([1,1′-biphenyl]-4-ylcarboxamido)phenyl)-1,2,4-oxadiazol-5-yl)-2,2-dimethylbutanoic acid

The compound of example 338 was prepared analogous to the compound of example 7 by hydrolysis of the compound of example 337. Yield: 54%; ¹H NMR (DMSO-d₆, 300 MHz): δ 12.41 (s, 1H), 10.59 (s, 1H), 8.11 (d, 2H), 8.02 (s, 4H), 7.88 (d, 2H), 7.91 (m, 2H), 7.55 (m, 2H), 7.46 (m, 1H), 2.99 (m, 2H), 2.04 (m, 2H), 1.18 (s, 6H); MS: m/z 456 (M+1).

### Example 339

### Ethyl 4-(2-(tert-butoxy)-2-oxoethylidene)cyclohexanecarboxylate

NaH (282 mg, 1.2 eq) was washed with petroleum ether, suspended in THF (10 mL), cooled to 0° C. and t-butyl diethyl phosphonoacetate (2.22 g, 1.5 eq) in THF (5 mL) was added dropwise. The resulting solution was stirred for 1 h at 0° C. followed by addition of a solution of ethyl-4-oxocyclohexane carboxylate (1 g, 1.0 eq) in THF (5 mL) dropwise. The temperature was raised slowly to room temperature and stirred for 16 h. After completion of the reaction, the solvent was removed, water was added and the resulting mixture was extracted with ethylacetate. The organic layer was washed with water and concentrated to yield a residue, which was purified by column chromatography (silica gel, 1-5% ethyl acetate in petrolium ether) to afford the title compound. Yield: 1.25 g (79%); ¹H NMR (CDCl₃; 300 MHz): δ 5.58 (s, 1H), 4.18 (q, 2H), 3.65 (m, 1H), 2.61 (m, 1H), 2.35 (m, 1H), 2.22 (m, 2H), 2.10 (m, 2H), 1.78 (m, 2H), 1.61 (m, 1H), 1.50 (s, 9H), 1.20 (t, 3H); MS: m/z 290.7 (M+Na).

### Example 340

### Ethyl 4-(2-(tert-butoxy)-2-oxoethyl)cyclohexanecarboxylate

In a Parr shaker apparatus, ethyl 4-(2-tert-butoxy-2-oxoethylidene)cyclohexane carboxylate (1.25 g) was dissolved in ethyl acetate (50 mL), palladium on charcoal (0.125 g) was added and the reaction mixture was stirred at room temperature, at 50 psi pressure of hydrogen for 3 h. After completion of the reaction, the reaction mixture was filtered through Celite® and concentrated to afford the title compound. Yield: 1.1 g (87%); ¹H NMR (CDCl₃, 300 MHz): δ 4.07 (q, 2H), 2.16 (m, 1H), 2.05 (d, 2H), 1.86 (m, 2H), 1.70 (m, 1H), 1.48 (m, 2H), 1.36 (s, 9H), 1.30 (m, 2H), 1.15 (m, 4H), 1.01 (m, 1H); MS: m/z 271.2 (M+1), 293.2 (M+Na).

### Example 341

### 4-(2-(tert-Butoxy)-2-oxoethyl)cyclohexanecarboxylic acid

The compound of example 340 (10 g, 1.0 eq) was dissolved in a mixture of MeOH:H₂O (400 mL:100 mL) and to this solution, 2.5 M KOH (26.9 mL, 2.0 eq) was added and the reaction mixture was stirred at room temperature for 16 h. After completion of the reaction, the reaction mixture was acidified to pH of 1 by addition of dilute HCl, methanol was removed and then extracted with ethyl acetate. The organic layer was washed with water, dried over sodium sulfate and concentrated to obtain an oily compound, which was solidified by stirring it with petroleum ether at 20° C. The solid obtained was filtered and dried to afford the title compound. Yield: 1.8 g (20%); ¹H NMR (CDCl₃; 300 MHz): δ 12.02 (s, 1H), 2.12 (m, 1H), 2.07 (d, 2H), 1.88 (m, 2H), 1.72 (m, 2H), 1.60 (m, 1H), 1.39 (s, 9H), 1.35 (m, 2H), 1.03 (m, 2H); MS: m/z 265.2 (M+Na).

### Example 342

### t-Butyl 2-(4-((2-(4-nitrophenyl)-2-oxoethyl)carbamoyl)cyclohexyl)acetate

To a solution of the compound of example 341 (1.97 g) in DMF (200 mL), were added compound of example 2 (2.114 g, 1.2 eq) and BOP (3.6 g, 1.0 eq). The reaction mixture was stirred for 5 min at room temperature and triethylamine (2.26 mL, 2.0 eq) was added. The reaction mixture was heated at 60° C. for 16 h. After completion of the reaction, the reaction mixture was cooled to room temperature, water was added and extracted with ethyl acetate. The organic layer was washed with water and concentrated to yield an oil, which was purified by column chromatography (silica gel, 1 ethyl acetate in CHCl₃) to yield an oil, which was stirred with diethyl ether to afford the title compound. Yield: 900 mg (27%); ¹H NMR (DMSO-d₆, 300 MHz): δ 8.36 (s, 2H), 8.23 (s, 1H), 8.20 (d, 2H), 4.59 (d, 2H), 2.17 (m, 1H), 2.08 (d, 2H), 1.76 (m, 4H), 1.60 (m, 1H), 1.39 (s, 9H), 1.32 (m, 2H), 1.02 (m, 2H); MS: m/z 405.2 (M+1), 427.2 (M+Na).

### Example 343

### t-Butyl 2-(4-(5-(4-nitrophenyl)thiazol-2-yl)cyclohexyl)acetate

To a solution of the compound of example 342 (2.0 g, 1.0 eq) in 1,4-dioxane (200 mL) was added Lawesson's reagent (2.60 g, 1.3 eq) and the reaction mixture was stirred at 60° C. for 3 h. After completion of the reaction, the solvent was removed and the crude residue was purified by column chromatography (silica gel, 3% ethyl acetate in CHCl₃) to afford the title compound. Yield: 1.25 g (63%); ¹H NMR (DMSO-d₆, 300 MHz): δ 8.34 (s, 1H), 8.28 (d, 2H), 7.93 (d, 2H), 3.00 (m, 1H), 2.50 (m, 1H), 2.14 (d, 2H), 2.12 (m, 1H), 1.80 (m, 2H), 1.75 (m, 1H), 1.60 (m, 2H), 1.41 (s, 9H), 1.20 (m, 2H); MS: m/z 403.2 (M+1), 425.2 (M+Na).

### Example 344

### t-Butyl 2-(4-(5-(4-aminophenyl)thiazol-2-yl)cyclohexyl)acetate

The compound of example 344 was prepared analogous to the compound of example 5 by reduction of the compound of example 343. Yield: 502 mg (72%); ¹H NMR (DMSO-d₆; 300 MHz): δ 7.74 (s, 1H), 7.27 (d, 2H), 6.56 (d, 2H), 5.27 (s, 2H), 2.89 (m, 1H), 2.15 (d, 2H), 2.06 (m, 2H), 1.81 (m, 2H), 1.73 (m, 1H), 1.55 (m, 2H), 1.41 (s, 9H), 1.23 (m, 2H); MS: m/z 373.2 (M+1).

### Example 345

### t-Butyl 2-(4-(5-(4-(3-(2-chlorophenyl)ureido)phenyl)thiazol-2-yl)cyclohexyl)acetate

The compound of example 345 was prepared analogous to the compound of example 6 by reaction of the compound of example 344 with 2-chloro-1-isocyanatobenzene.

Yield: 143 mg (81%); ¹H NMR (DMSO-d₆, 300 MHz): δ 9.56 (s, 1H), 8.34 (s, 1H), 8.17 (d, 1H), 7.95 (s, 1H), 7.57 (d, 2H), 7.53 (d, 2H), 7.48 (d, 1H), 7.33 (t, 1H), 7.06 (t, 1H), 2.94 (m, 1H), 2.13 (d, 2H), 2.08 (m, 2H), 1.82 (m, 2H), 1.74 (m, 1H), 1.57 (m, 2H), 1.41 (s, 9H), 1.20 (m, 2H); MS: m/z 526.2 (M+1).

### Example 346

### 2-(4-(5-(4-(3-(2-Chlorophenyl)ureido)phenyl)thiazol-2-yl)cyclohexyl)acetic acid

To a solution of the compound of example 345 (90 mg, 1.0 eq) in THF (5 mL) and MeOH (2.5 mL), was added 1N NaOH solution (0.85 mL, 5.0 eq) and the reaction mixture was stirred at 60° C. for 16 h. The solvent was removed, water was added and the reaction mixture was acidified with dilute HCl to obtain a solid, which was filtered, washed with acetone and dried to afford the title compound. Yield: 15 mg (18%); ¹H NMR (DMSO-d₆, 300 MHz): δ 9.67 (s, 1H), 8.15 (d, 1H), 7.96 (d, 1H), 7.57 (m, 5H), 7.47 (d, 1H), 7.33 (t, 1H), 7.16 (t, 1H), 2.94 (m, 1H), 2.12 (d, 2H), 2.08 (m, 2H), 1.86 (m, 2H), 1.74 (m, 1H), 1.56 (m, 2H), 1.19 (m, 2H); MS: m/z 470.1 (M+1).

### Example 347

### t-Butyl 2-(4-(5-(4-(3-(2-fluorophenyl)ureido)phenyl)thiazol-2-yl)cyclohexyl)acetate

The compound of example 347 was prepared analogous to the compound of example 6 by reaction of the compound of example 344 with 2-fluoro-1-isocyanatobenzene.

Yield: 77%; ¹H NMR (DMSO-d₆; 300 MHz): δ 9.22 (s, 1H), 8.15 (t, 1H), 7.94 (d, 1H), 7.53 (m, 5H), 7.24 (t, 1H), 7.14 (t, 1H), 7.02 (m, 1H), 2.90 (m, 1H), 2.13 (d, 2H), 2.08 (m, 2H), 1.82 (m, 2H), 1.71 (m, 1H), 1.53 (m, 2H), 1.41 (5, 9H), 1.20 (m, 2H); MS: m/z 510.1 (M+1).

### Example 348

### 2-(4-(5-(4-(3-(2-Fluorophenyl)ureido)phenyl)thiazol-2-yl)cyclohexyl)acetic acid

To a solution of the compound of formula 347 (90 mg, 1.0 eq) in dichloromethane (5 mL) was added, trifluoroacetic acid (0.1 mL, 5.0 eq) and the reaction mixture was stirred at room temperature for 16 h. After completion of the reaction, dichloromethane was removed and the reaction mixture was stirred in ether and the solid was filtered, washed with acetone and dried to afford the title compound. Yield: 55 mg (65%); ¹H NMR (DMSO-d₆; 300 MHz): δ 12.03 (bs, 1H), 9.20 (s, 1H), 8.14 (s, 2H), 7.49 (bs, 4H), 7.13 (m, 4H), 2.91 (m, 1H), 2.12 (d, 2H), 2.10 (m, 4H), 1.81 (m, 1H), 1.47 (m, 2H), 1.13 (m, 2H); MS: m/z 454.2 (M+1).

### Example 349

### 4-Oxocyclohexanecarboxylic acid

Ethyl 4-oxocyclohexanecarboxylate (5.0 g, 29.4 mmol) was heated to reflux in ethanol (30 mL) with 10% NaOH (10 mL) for 2 h. The reaction mixture was cooled and concentrated to obtain a residue, which was washed with ethyl acetate, acidified with concentrated HCl and extracted with ethyl acetate. The organic layer was dried over anhydrous sodium sulfate and the solvent was evaporated to afford the title compound.

Yield: 3.35 g (80%); ¹H NMR (DMSO-d₆; 300 MHz): δ 12.32 (bs, 1H), 2.73 (m, 1H), 2.41 (m, 2H), 2.24 (m, 2H), 2.09 (m, 2H), 1.82 (m, 2H); MS: m/z 141.0 (M−1).

### Example 350

### 4-(2-Ethoxy-2-oxoethylidene)cyclohexanecarboxylic acid

4-oxocyclohexanecarboxylic acid (2 g, 14.07 mmol) was dissolved in 20 mL of anhydrous ethanol and 21 wt. percent sodium ethoxide in ethanol (5.4 mL, 1.15 g, 17 mmol, 1.2 eq) was added followed by ethyl 2-(diethoxyphosphoryl)acetate (3.47 g, 15.5 mmol) under an atmosphere of nitrogen. The reaction mixture was cooled in an ice bath to 4° C. and 21 wt. percent sodium ethoxide in ethanol (5.0 mL, 1.05 g, 15.4 mmol, 1.1 eq) was added at such a rate that the temperature remained between 4-5° C. After the addition, the ice bath was removed, and the reaction was stirred for 1 h. The reaction pH was adjusted to pH of 5 with glacial acetic acid (1.94 g, 2.3 eq), solvent was removed by evaporation and the remaining oil was partitioned between isopropyl ether (35 mL) and 1 M hydrochloric acid (35 mL). The organic phase was separated, washed with water (35 mL), brine (35 mL), dried with sodium sulfate and solvent was evaporated to afford the title compound. Yield: 2.3 g (77%): ¹H NMR (DMSO-d₆, 300 MHz): δ 12.17 (bs, 1H), 5.62 (s, 1H), 4.10 (q, 2H), 3.45 (m, 1H), 2.51 (m, 1H), 2.30 (m, 3H), 1.97 (m, 2H), 1.54 (m, 2H), 1.20 (t, 3H); MS: m/z 211.1 (M−1).

### Example 351

### trans-4-(2-Ethoxy-2-oxoethyl)cyclohexanecarboxylic acid

To a solution of the compound of example 350 (5 g, 23.56 mmol) in ethanol (50 mL), 500 mg Pd/C (10% by wt) was added and the reaction mixture was heated to 30° C. To the reaction mixture, ammonium formate (3.7 g) was added while continuing to heat to 50° C. The mixture was stirred at 50° C. for 45 min, cooled to 10° C. to 15° C. and filtered over Celite®. The resultant filtrate was concentrated to a low volume to remove ethanol, diluted with isopropylether (50 mL) and 1 N HCl (50 mL). The mixture was stirred, allowed to settle, and the organic layer was separated. The organic layer was washed with water (5 volumes) and brine (10 volumes) and dried over sodium sulfate. The organic layers were concentrated to afford the title compound as a mixture of cis and trans isomers. Yield: 4.7 g (93%)

The oil obtained as mixture of isomers (5 g, 23.34 mmol) was taken in n-hexane (22 mL) and refluxed for 1 h and slowly cooled to room temperature, then further cooled to 15° C. when a solid precipitated out. The reaction mixture was stirred at room temperature for 1 h and the solid obtained was filtered and dried at 40° C. to afford the title compound as trans isomer. Yield: 2.2 g (44%); ¹H NMR (DMSO-d₆; 300 MHz): δ 11.99 (bs, 1H), 4.02 (q, 2H), 2.14 (d, 2H), 2.10 (m, 1H), 1.87 (m, 2H), 1.70 (m, 2H), 1.60 (m, 1H), 1.28 (m, 2H), 1.16 (t, 3H), 0.97 (m, 2H); MS: m/z 215.1 (M+1), 237.1 (M+Na).

### Example 352

### Ethyl 2-(4-(2-(4-nitrophenyl)-2-oxoethylcarbamoyl)cyclohexyl)acetate

To a solution of the compound of example 351 (11 g, 51.3 mmol) in DMF (110 mL) was added HATU (21.47 g, 56.5 mmol), 2-amino-1-(4-nitrophenyl)ethanone hydrochloride (13.35 g, 61.6 mmol) and DIPEA (26.9 mL, 154 mmol) and the reaction mixture was stirred at room temperature for 3-4 h. After completion of the reaction, water was added and extracted with ethyl acetate. The organic layer was washed with water and concentrated. The resulting solid was stirred in methanol and filtered to afford the title compound. Yield: 10.8 g (56%); ¹H NMR (DMSO-d₆, 300 MHz): δ 8.33 (d, 2H), 8.17 (d, 2H), 4.58 (d, 2H), 4.05 (q, 2H), 2.16 (d, 2H), 2.15 (m, 1H), 1.68 (m, 4H), 1.60 (m, 1H), 1.32 (m, 2H), 1.17 (t, 3H), 0.97 (m, 2H); MS: m/z 377.2 (M+1), 399.2 (M+Na).

### Example 353

### Ethyl 2-(4-(5-(4-nitrophenyl)thiazol-2-yl)cyclohexyl)acetate

To a solution of the compound of example 352 (10.5 g, 27.9 mmol) in 1,4 dioxane (210 mL) was added Lawesson's Reagent (12.41 g, 30.7 mmol) and the reaction mixture was stirred at 55° C. for 3 h. After completion of the reaction, the reaction mixture was cooled to room temperature, basified with saturated solution of NaHCO₃ and extracted with ethyl acetate (50 mL×3). The combined organic layer was washed with water and brine and the solvent was removed to yield a solid. The resulting solid compound was stirred in methanol (30 mL), filtered and dried to afford the title compound. Yield: 8.5 g (77%); ¹H NMR (DMSO-d₆, 300 MHz): δ 8.31 (s, 1H), 8.25 (d, 2H), 7.90 (d, 2H), 4.07 (q, 2H), 2.98 (m, 1H), 2.21 (d, 2H), 2.11 (m, 2H), 1.81 (m, 2H), 1.73 (m, 1H), 1.52 (m, 2H), 1.81 (t, 3H), 1.11 (m, 2H); MS m/z 375.1 (M+1).

### Example 354

### Ethyl 2-(4-(5-(4-aminophenyl)thiazol-2-yl)cyclohexyl)acetate

The compound of example 354 was prepared analogous to the compound of example 5 by reduction of the compound of example 353. Yield: 6.3 g (82%); ¹H NMR (DMSO-d₆, 300 MHz): δ 7.69 (s, 1H), 7.24 (d, 2H), 6.56 (d, 2H), 5.33 (s, 2H), 4.05 (q, 2H), 2.87 (m, 1H), 2.20 (d, 2H), 2.07 (m, 2H), 1.79 (m, 2H), 1.71 (m, 1H), 1.51 (m, 2H), 1.18 (t, 3H), 1.13 (m, 2H); MS: m/z 345.2 (M+1).

### Example 355

### Ethyl 2-(4-(5-(4-(3-(3,5-difluorophenyl)ureido)phenyl)thiazol-2-yl)cyclohexyl)acetate

The compound of example 355 was prepared analogous to the compound of example 6 by reaction of the compound of example 354 with 3,5-difluoro-1-isocyanatobenzene.

Yield: 86%; ¹H NMR (DMSO-d₆, 300 MHz): δ 9.06 (bs, 2H), 7.92 (m, 1H), 7.54 (d, 2H), 7.49 (d, 2H), 7.18 (d, 2H), 6.80 (t, 1H), 4.07 (q, 2H), 2.92 (m, 1H), 2.21 (d, 2H), 2.09 (m, 2H), 1.80 (m, 2H), 1.71 (m, 1H), 1.54 (m, 2H), 1.18 (t, 3H), 1.14 (m, 2H); MS: m/z 500 (M+1).

### Example 356

### 2-(4-(5-(4-(3-(3,5-Difluorophenyl)ureido)phenyl)thiazol-2-yl)cyclohexyl)acetic acid

The compound of example 356 was prepared analogous to the compound of example 346 by hydrolysis of the compound of example 355. Yield: 750 mg (63%); ¹H NMR (DMSO-d₆, 300 MHz): δ 9.51 (s, 1H), 9.29 (s, 1H), 7.95 (s, 1H), 7.55 (d, 2H), 7.49 (d, 2H), 7.17 (d, 1H), 6.80 (m, 1H), 2.94 (m, 1H), 2.13 (m, 4H), 1.82 (m, 2H), 1.73 (m, 2H), 1.54 (m, 2H), 1.17 (m, 2H); MS: m/z 472 (M+1).

### Example 357

### Ethyl 2-(4-(5-(4-(3-(2,4,5-trifluorophenyl)ureido)phenyl)thiazol-2-yl)cyclohexyl)acetate

The compound of example 357 was prepared analogous to the compound of example 6 by reaction of the compound of example 354 with 2,4,5-trifluoro-1-isocyanatobenzene. Yield: 74%; ¹H NMR (DMSO-d₆, 300 MHz): δ 9.20 (s, 1H), 8.73 (s, 1H), 8.22 (m, 1H), 7.93 (s, 1H), 7.66 (m, 1H), 7.54 (d, 2H), 7.49 (d, 2H), 4.07 (q, 2H), 2.92 (m, 1H), 2.21 (d, 2H), 2.09 (m, 2H), 1.80 (m, 2H), 1.69 (m, 1H), 1.54 (m, 2H), 1.18 (t, 3H), 1.11 (m, 2H); MS: m/z 518 (M+1).

### Example 358

### 2-(4-(5-(4-(3-(2,4,5-Trifluorophenyl)ureido)phenyl)thiazol-2-yl)cyclohexyl)acetic acid

The compound of example 358 was prepared analogous to the compound of example 346 by hydrolysis of the compound of example 357. Yield: 89%; ¹H NMR (DMSO-d₆, 300 MHz): δ 9.47 (s, 1H), 8.85 (s, 1H), 8.19 (m, 1H), 7.95 (s, 1H), 7.66 (m, 1H), 7.55 (d, 2H), 7.50 (d, 2H), 2.89 (m, 1H), 2.13 (d, 2H), 2.06 (m, 2H), 1.83 (m, 2H), 1.69 (m, 1H), 1.51 (m, 2H), 1.18 (m, 2H); MS: m/z 490 (M+1).

### Example 359

### Ethyl 2-(4-(5-(4-(3-(2,4,6-trifluorophenyl)ureido)phenyl)thiazol-2-yl)cyclohexyl)acetate

The compound of example 359 was prepared analogous to the compound of example by reaction of the compound of example 354 with 2,4,6-trifluoro-1-isocyanatobenzene. Yield: 73%; ¹H NMR (CDCl₃, 300 MHz): δ 7.71 (s, 1H), 7.41 (d, 2H), 7.32 (d, 2H), 7.22 (s, 1H), 6.70 (t, 2H), 6.49 (s, 1H), 4.17 (q, 2H), 2.91 (m, 1H), 2.25 (d, 2H), 2.21 (m, 2H), 1.93 (m, 2H), 1.85 (m, 1H), 1.58 (m, 2H), 1.28 (t, 3H), 1.19 (m, 2H); MS: m/z 518 (M+1).

### Example 360

### 2-(4-(5-(4-(3-(2,4,6-Trifluorophenyl)ureido)phenyl)thiazol-2-yl)cyclohexyl)acetic acid

The compound of example 360 was prepared analogous to the compound of example 346 by hydrolysis of the compound of example 359. Yield: 73%; ¹H NMR (CDCl₃, 300 MHz): δ 12.03 (s, 1H), 9.13 (s, 1H), 8.06 (d, 1H), 7.91 (s, 1H), 7.52 (d, 2H), 7.48 (d, 2H), 7.27 (t, 2H), 2.91 (m, 1H), 2.13 (d, 2H), 2.05 (m, 2H), 1.82 (m, 2H), 1.69 (m, 1H), 1.53 (m, 2H), 1.17 (m, 2H); MS: m/z 490 (M+1).

### Example 361

### Ethyl 2-(4-(5-(4-(3-(2,4-difluorophenyl)ureido)phenyl)thiazol-2-yl)cyclohexyl)acetate

The compound of example 361 was prepared analogous to the compound of example 6 by reaction of the compound of example 354 with 2,4-difluoro-1-isocyanatobenzene.

Yield: 82%; ¹H NMR (CDCl₃, 300 MHz): δ 8.04 (m, 1H), 7.75 (s, 1H), 7.48 (d, 2H), 7.40 (d, 2H), 7.12 (s, 1H), 6.93 (m, 3H), 4.18 (q, 2H), 2.97 (m, 1H), 2.26 (d, 2H), 2.18 (m, 2H), 1.94 (m, 2H), 1.85 (m, 1H), 1.57 (m, 2H), 1.29 (t, 3H), 1.20 (m, 2H); MS: m/z 500.2 (M+1).

### Example 362

### 2-(4-(5-(4-(3-(2,4-Difluorophenyl)ureido)phenyl)thiazol-2-yl)cyclohexyl)acetic acid

The compound of example 362 was prepared analogous to the compound of example 346 by hydrolysis of the compound of example 361. Yield: 77%; ¹H NMR (CDCl₃, 300 MHz): δ 9.38 (s, 1H), 8.63 (s, 1H), 8.09 (m, 1H), 7.95 (s, 1H), 7.55 (d, 2H), 7.50 (d, 2H), 7.33 (m, 1H), 7.06 (m, 1H), 2.94 (m, 1H), 2.14 (d, 2H), 2.07 (m, 2H), 1.83 (m, 2H), 1.73 (m, 1H), 1.55 (m, 2H), 1.19 (m, 2H); MS: m/z 472.2 (M+1).

### Example 363

### Ethyl 2-(4-(5-(4-(2,4-dichlorobenzamido)phenyl)thiazol-2-yl)cyclohexyl)acetate

The compound of example 363 was prepared analogous to the compound of example 14 by reaction of the compound of example 354 with 2,4-dichlorobenzoyl chloride.

Yield: 80%; ¹H NMR (CDCl₃, 300 MHz): δ 7.97 (s, 1H), 7.79 (s, 1H), 7.76 (d, 1H), 7.68 (d, 2H), 7.55 (d, 2H), 7.49 (d, 1H), 7.40 (dd, 1H), 4.17 (q, 2H), 2.98 (m, 1H), 2.25 (d, 2H), 2.19 (m, 2H), 1.95 (m, 2H), 1.85 (m, 1H), 1.67 (m, 2H), 1.29 (t, 3H), 1.21 (m, 2H); MS: m/z 517 (M+1).

### Example 364

### 2-(4-(5-(4-(2,4-Dichlorobenzamido)phenyl)thiazol-2-yl)cyclohexyl)acetic acid

The compound of example 362 was prepared analogous to the compound of example 346 by hydrolysis of the compound of example 361. Yield: 83%; ¹H NMR (DMSO-d₆, 300 MHz): δ 12.06 (s, 1H), 10.66 (s, 1H), 7.98 (s, 1H), 7.76 (d, 1H), 7.75 (d, 2H), 7.65 (d, 1H), 7.61 (d, 2H), 7.56 (dd, 1H), 2.98 (m, 1H), 2.14 (d, 2H), 2.07 (m, 2H), 1.83 (m, 2H), 1.71 (m, 1H), 1.55 (m, 2H), 1.18 (m, 2H); MS: m/z 489.1 (M+1).

### Example 365

### Ethyl 2-(4-(5-(4-(2-fluoro-6-(trifluoromethyl)benzamido)phenyl)thiazol-2-yl)cyclohexyl)acetate

The compound of example 365 was prepared analogous to the compound of example 14 by reaction of the compound of example 354 with 2-fluoro-6-trifluoromethylbenzoyl chloride. Yield: 58%; ¹H NMR (CDCl₃, 300 MHz): δ 7.78 (s, 1H), 7.65 (d, 2H), 7.60 (m, 1H), 7.57 (m, 2H), 7.54 (d, 2H), 7.42 (m, 1H), 4.17 (q, 2H), 2.98 (m, 1H), 2.25 (d, 2H), 2.19 (m, 2H), 1.94 (m, 2H), 1.85 (m, 1H), 1.68 (m, 2H), 1.29 (t, 3H), 1.20 (m, 2H); MS: m/z 535 (M+1).

### Example 366

### 2-(4-(5-(4-(2-Fluoro-6-(trifluoromethyl)benzamido)phenyl)thiazol-2-yl)cyclohexyl)acetic acid

The compound of example 366 was prepared analogous to the compound of example 346 by hydrolysis of the compound of example 365. Yield: 63%; ¹H NMR (CDCl₃, 300 MHz): δ 12.05 (s, 1H), 10.93 (s, 1H), 7.98 (s, 1H), 7.77 (m, 3H), 7.70 (d, 2H), 7.62 (d, 2H), 2.94 (m, 1H), 2.14 (d, 2H), 2.11 (m, 2H), 1.83 (m, 2H), 1.70 (m, 1H), 1.55 (m, 2H), 1.18 (m, 2H); MS: m/z 507.1 (M+1).

### Example 367

### Ethyl 2-(4-(5-(4-(3-(3,4,5-trifluorophenyl)ureido)phenyl)thiazol-2-yl)cyclohexyl)acetate

To a solution of the compound of example 354 (1.5 g, 4.35 mmol) in dichloromethane (60 mL) were added triphosgene (0.775 g, 2.61 mmol) and triethylamine (1.214 mL, 8.71 mmol) and the reaction mixture was stirred for 2 h at room temperature. 3,4,5-trifluoroaniline (0.641 g, 4.35 mmol) was added and stirred for 16 h at room temperature. After completion of the reaction, water was added and the reaction mixture was extracted with dichloromethane (60 mL×2). The organic layer was washed with water and concentrated to yield a residue, which was further purified by column chromatography (silica gel, 10% ethyl acetate in petroleum ether) to afford the title compound. Yield: 350 mg (15%); ¹H NMR (DMSO-d₆, 300 MHz): δ 9.03 (s, 1H), 9.01 (s, 1H), 7.92 (s, 1H), 7.54 (d, 2H), 7.49 (d, 2H), 7.39 (m, 2H), 4.07 (q, 2H), 2.88 (m, 1H), 2.21 (d, 2H), 2.09 (m, 2H), 1.80 (m, 2H), 1.72 (m, 1H), 1.55 (m, 2H), 1.18 (t, 3H), 1.11 (m, 2H); MS: m/z 515.5 (M−1).

### Example 368

### 2-(4-(5-(4-(3-(3,4,5-Trifluorophenyl)ureido)phenyl)thiazol-2-yl)cyclohexyl)acetic acid

The compound of example 368 was prepared analogous to the compound of example 346 by hydrolysis of the compound of example 367. Yield: 67%; ¹H NMR (DMSO-d₆, 300 MHz): δ 9.55 (s, 1H), 9.36 (s, 1H), 7.97 (s, 1H), 7.54 (d, 2H), 7.49 (d, 2H), 7.37 (m, 2H), 2.94 (m, 1H), 2.13 (d, 2H), 2.06 (m, 2H), 1.82 (m, 2H), 1.69 (m, 1H), 1.54 (m, 2H), 1.17 (m, 2H); MS: m/z 490 (M+1).

### Example 369

### Ethyl 2-(4-(5-(4-(2-phenyl-5-(trifluoromethyl)oxazole-4-carboxamido)phenyl)thiazol-2-yl)cyclohexyl)acetate

To a solution of commercially available 2-phenyl-5-(trifluoromethyl)oxazole-4-carboxylic acid (179 mg, 0.697 mmol) in DMF (10 ml) was added HATU (243 mg, 0.639 mmol) and the reaction mixture was stirred for 10 min. The compound of example 354 (200 mg, 0.581 mmol) and DIPEA (0.203 mL, 1.161 mmol) were added and the reaction mixture was stirred for 5 h. After completion of the reaction, water was added and the reaction mixture was extracted with ethyl acetate. The organic layer was washed with water and brine and concentrated to yield a crude residue, which was purified by column chromatography (silica gel, 20% ethylacetate in chloroform) to afford the title compound. Yield: 205 mg (60%); ¹H NMR (CDCl₃, 300 MHz): δ 9.01 (s, 1H), 8.17 (dd, 2H), 7.82 (s, 1H), 7.81 (d, 2H), 7.63 (m, 3H), 7.57 (d, 2H), 4.20 (q, 2H), 3.01 (m, 1H), 2.28 (d, 2H), 2.22 (m, 2H), 1.97 (m, 2H), 1.90 (m, 1H), 1.70 (m, 2H), 1.31 (t, 3H), 1.23 (m, 2H); MS: m/z 584.2 (M+1).

### Example 370

### 2-(4-(5-(4-(2-Phenyl-5-(trifluoromethyl)oxazole-4-carboxamido) phenyl)thiazol-2-VI) cyclohexyl)acetic acid

The compound of example 370 was prepared analogous to the compound of example 346 by hydrolysis of the compound of example 369. Yield: 65%; ¹H NMR (DMSO-d₆, 300 MHz): δ 12.08 (s, 1H), 10.71 (s, 1H), 8.17 (dd, 2H), 8.03 (s, 1H), 7.91 (d, 2H), 7.69 (m, 3H), 7.65 (d, 2H), 2.96 (m, 1H), 2.16 (d, 2H), 2.08 (m, 2H), 1.85 (m, 2H), 1.72 (m, 1H), 1.57 (m, 2H), 1.51 (m, 2H); MS: m/z 556.2 (M+1).

### Example 371

### Ethyl 2-(4-(5-(4-(5-methyl-2-phenyloxazole-4-carboxamido)phenyl)thiazol-2-yl)cyclohexyl)acetate

The compound of example 371 was prepared analogous to the compound of example 369 by reaction of the compound of example 354 with 5-methyl-2-phenyloxazole-4-carboxylic acid. Yield: 88%; ¹H NMR (DMSO-d₆, 300 MHz): δ 10.12 (s, 1H), 8.01 (m, 2H), 8.02 (s, 1H), 7.94 (d, 2H), 7.63 (d, 2H), 7.59 (m, 3H), 4.11 (q, 2H), 2.96 (m, 1H), 2.73 (s, 3H), 2.24 (d, 2H), 2.13 (m, 2H), 1.84 (m, 2H), 1.72 (m, 1H), 1.58 (m, 2H), 1.22 (t, 3H), 1.17 (m, 2H); MS: m/z 530.2 (M+1).

### Example 372

### 2-(4-(5-(4-(5-Methyl-2-phenyloxazole-4-carboxamido)phenyl)thiazol-2-yl)cyclohexyl)acetic acid

The compound of example 372 was prepared analogous to the compound of example 346 by hydrolysis of the compound of example 371. Yield: 82%; ¹H NMR (DMSO-d₆, 300 MHz): δ 10.12 (s, 1H), 8.01 (m, 2H), 8.02 (s, 1H), 7.94 (d, 2H), 7.63 (d, 2H), 7.59 (m, 3H), 2.96 (m, 1H), 2.73 (s, 3H), 2.17 (d, 2H), 2.10 (m, 2H), 1.86 (m, 2H), 1.76 (m, 1H), 1.58 (m, 2H), 1.21 (m, 2H); MS: m/z 502.2 (M+1).

### Example 373

### Ethyl 2-(4-(5-(4-(3-(2-fluorophenyl)thioureido)phenyl)thiazol-2-yl)cyclohexyl)acetate

The compound of example 373 was prepared analogous to the compound of example 6 by reaction of the compound of example 354 with 2-fluoro-1-isothiocyanato benzene.

Yield: 82%; ¹H NMR (DMSO-d₆, 300 MHz): δ 10.08 (s, 1H), 9.57 (s, 1H), 8.01 (s, 1H), 7.58 (m, 5H), 7.28 (d, 2H), 7.21 (m, 1H), 4.10 (q, 2H), 2.96 (m, 1H), 2.24 (d, 2H), 2.13 (m, 2H), 1.83 (m, 2H), 1.76 (m, 1H), 1.58 (m, 2H), 1.21 (t, 3H), 1.14 (m, 2H); MS: m/z 498.2 (M+1).

### Example 374

### 2-(4-(5-(4-(3-(2-Fluorophenyl)thioureido)phenyl)thiazol-2-yl)cyclohexyl)acetic acid

The compound of example 374 was prepared analogous to the compound of example 346 by hydrolysis of the compound of example 373. Yield: 59%; ¹H NMR (DMSO-d₆, 300 MHz): δ 12.06 (s, 1H), 10.08 (s, 1H), 9.57 (s, 1H), 8.01 (s, 1H), 7.59 (m, 5H), 7.28 (m, 3H), 2.96 (m, 1H), 2.16 (d, 2H), 2.09 (m, 2H), 1.85 (m, 2H), 1.75 (m, 1H), 1.58 (m, 2H), 1.23 (m, 2H); MS: m/z 470.1 (M+1).

### Example 375

### Ethyl 2-(4-(5-(4-(3-(2-fluorophenyl)guanidino)phenyl)thiazol-2-yl)cyclohexyl)acetate

The compound of example 375 was prepared analogous to the compound of example 268 by reaction of the compound of example 373 with methanolic ammonia and mercuric oxide yellow. Yield: 53%; ¹H NMR (DMSO-d₆, 300 MHz): δ 8.38 (s, 1H), 7.89 (s, 1H), 7.61 (s, 2H), 7.48 (d, 2H), 7.11 (m, 3H), 6.95 (m, 2H), 5.27 (s, 1H), 4.10 (q, 2H), 2.93 (m, 1H), 2.26 (d, 2H), 2.12 (m, 2H), 1.82 (m, 2H), 1.76 (m, 1H), 1.56 (m, 2H), 1.21 (t, 3H), 1.14 (m, 2H); MS: m/z 481.3 (M+1).

### Example 376

### 2-(4-(5-(4-Nitrophenyl)thiazol-2-yl)cyclohexyl)acetohydrazide

A mixture of the compound of example 353 (3.2 g, 8.55 mmol) and hydrazine hydrate (42.8 g, 855 mmol) was stirred at 80° C. for 15 min followed by addition of ethanol (25 mL). This reaction mixture was then stirred at 80° C. for an additional 4-5 h. After completion of the reaction, mixture was cooled to room temperature. The precipitated solid was filtered and dried to afford the title compound. Yield 2.3 g (72%); ¹H NMR (DMSO-d₆, 300 MHz): δ 8.94 (s, 1H), 8.32 (s, 1H), 8.26 (d, 2H), 7.91 (d, 2H), 4.15 (s, 2H), 3.00 (m, 1H), 2.12 (m, 2H), 1.94 (d, 2H), 1.78 (m, 3H), 1.50 (m, 2H), 1.11 (m, 2H); MS: m/z 361.1 (M+1).

### Example 377

### 2-Methyl-5-((4-(5-(4-nitrophenyl)thiazol-2-yl)cyclohexyl)methyl)-1,3,4-oxadiazole

To a solution of the compound of example 376 (800 mg, 2.220 mmol) in POCl₃ (10 mL) was added acetic acid (0.190 mL, 3.33 mmol) and the reaction mixture was stirred for 3 h at 80-85° C. Following its completion, the reaction mass was cooled to room temperature, quenched in ice, stirred with a saturated solution of NaHCO₃ to neutralize POCl₃. The reaction mixture was then extracted with ethyl acetate and the combined organic layers were washed with water and concentrated to yield a yellow solid. This solid was further purified using flash column chromatography to afford the title compound. Yield: 400 mg (46° A)); ¹H NMR (CDCl₃; 300 MHz): δ 8.26 (d, 2H), 7.97 (s, 1H), 7.68 (d, 2H), 3.05 (m, 1H), 2.79 (d, 2H), 2.52 (s, 3H), 2.27 (m, 2H), 1.99 (m, 3H), 1.69 (m, 2H), 1.40 (m, 2H); MS: m/z 385.1 (M+1).

### Example 378

### 4-(2-(4-((5-Methyl-1,3,4-oxadiazol-2-yl)methyl)cyclohexyl)thiazol-5-yl)aniline

To a solution of the compound of example 377 (320 mg, 0.832 mmol) in ethanol (10 mL), water (5 mL) and THF (5 mL) were added iron (372 mg, 6.66 mmol) and ammonium chloride (356 mg, 6.66 mmol). The reaction mixture was stirred at 75° C. for 3 h. Following its completion the reaction mass was cooled to room temperature, filtered through Celite® and concentrated. Saturated NaHCO₃ solution was added to this reaction mixture and the compound was extracted with ethyl acetate. The organic layers were washed with water and concentrated. The compound was separated by flash column chromatography using 15% ethyl acetate in chloroform to afford the title compound. Yield: 180 mg (15° A)); ¹H NMR (DMSO-d6; 300 MHz): δ 7.70 (5, 1H), 7.24 (d, 2H), 6.57 (d, 2H), 5.34 (5, 2H), 2.90 (m, 1H), 2.74 (d, 2H), 2.44 (5, 3H), 2.09 (m, 2H), 1.81 (m, 3H), 1.53 (m, 2H), 1.25 (m, 2H); MS: m/z 355.2 (M+1).

### Example 379

### 1-(2,4-Difluorophenyl)-3-(4-(2-(4-((5-methyl-1,3,4-oxadiazol-2-yl)methyl)cyclohexyl)thiazol-5-yl)phenyl)urea

The compound of example 379 was prepared analogous to the compound of example 6 by reaction of the compound of example 378 with 2,4-di-fluorophenylisocyanate.

Yield: 69%; ¹H NMR (DMSO-d₆, 300 MHz): δ 9.15 (s, 1H), 8.52 (s, 1H), 8.09 (m, 1H), 7.93 (s, 1H), 7.54 (d, 2H), 7.49 (d, 2H), 7.34 (m, 1H), 7.06 (m, 1H), 2.95 (m, 1H), 2.75 (d, 2H), 2.44 (s, 3H), 2.11 (m, 2H), 1.83 (m, 3H), 1.56 (m, 2H), 1.26 (m, 2H); MS: m/z 510.2 (M+1).

### Example 380

### 1-(2-Chlorophenyl)-3-(4-(2-(4-((5-methyl-1,3,4-oxadiazol-2-yl)methyl)cyclohexyl)thiazol-5-yl)phenyl)urea

The compound of example 380 was prepared analogous to the compound of example 6 by reaction of the compound of example 378 with 2-chloro phenylisocyanate. Yield: 88%; ¹H NMR (DMSO-d₆, 300 MHz): δ 9.55 (s, 1H), 8.32 (s, 1H), 8.16 (d, 1H), 7.93 (s, 1H), 7.56 (d, 2H), 7.51 (d, 2H), 7.46 (dd, 1H), 7.29 (t, 1H), 7.05 (m, 1H), 2.95 (m, 1H), 2.75 (d, 2H), 2.44 (s, 3H), 2.12 (m, 2H), 1.83 (m, 3H), 1.56 (m, 2H), 1.27 (m, 2H); MS: m/z 508.2 (M+1).

### Example 381

### 1-(3,5-Difluorophenyl)-3-(4-(2-(4-((5-methyl-1,3,4-oxadiazol-2-yl)methyl)cyclohexyl)thiazol-5-yl)phenyl)urea

The compound of example 381 was prepared analogous to the compound of example 6 by reaction of the compound of example 378 with 3,5-difluorophenyl-1-isocyanatobenzene. Yield: 76%; ¹H NMR (DMSO-d₆; 300 MHz): δ 9.13 (s, 1H), 9.02 (s, 1H), 7.95 (s, 1H), 7.56 (d, 2H), 7.51 (d, 2H), 7.21 (d, 2H), 6.82 (m, 1H), 2.95 (m, 1H), 2.76 (d, 2H), 2.46 (s, 3H), 2.13 (m, 2H), 1.84 (m, 3H), 1.55 (m, 2H), 1.26 (m, 2H); MS: m/z 510.2 (M+1).

### Example 382

### 1-(4-(2-(4-((5-Methyl-1,3,4-oxadiazol-2-yl)methyl)cyclohexyl)thiazol-5-yl)phenyl)-3-(2,4,5-trifluorophenyl)urea

The compound of example 382 was prepared analogous to the compound of example 6 by reaction of the compound of example 378 with 2,4,5-trifluorophenylisocyanate.

Yield: 78%; ¹H NMR (DMSO-d₆, 300 MHz): δ 9.24 (s, 1H), 8.76 (s, 1H), 8.25 (m, 1H), 7.96 (s, 1H), 7.69 (m, 1H), 7.57 (d, 2H), 7.51 (d, 2H), 2.97 (m, 1H), 2.77 (d, 2H), 2.47 (s, 3H), 2.14 (m, 2H), 1.85 (m, 3H), 1.58 (m, 2H), 1.29 (m, 2H); MS: m/z 528.2 (M+1).

### Example 383

### 1-(4-(2-(4-((5-Methyl-1,3,4-oxadiazol-2-yl)methyl)cyclohexyl)thiazol-5-yl) phenyl)-3-(2,4,6-trifluorophenyl)urea

The compound of example 383 was prepared analogous to the compound of example 6 by reaction of the compound of example 378 with 2,4,6-trifluorophenylisocyanate.

Yield: 94%; ¹H NMR (DMSO-d₆, 300 MHz): δ 9.15 (s, 1H), 8.07 (s, 1H), 7.94 (s, 1H), 7.55 (m, 4H), 7.31 (t, 2H), 2.96 (m, 1H), 2.77 (d, 2H), 2.47 (s, 3H), 2.13 (m, 2H), 1.85 (m, 3H), 1.57 (m, 2H), 1.28 (m, 2H); MS: m/z 528.2 (M+1).

### Example 384

### 1-(4-(2-(4-((5-Methyl-1,3,4-oxadiazol-2-yl)methyl)cyclohexyl)thiazol-5-yl)phenyl)-3-phenylurea

The compound of example 384 was prepared analogous to the compound of example 6 by reaction of the compound of example 378 with phenylisocyanate. Yield: 53%; ¹H NMR (DMSO-d₆, 300 MHz): δ 8.83 (s, 1H), 8.70 (s, 1H), 7.94 (s, 1H), 7.55 (m, 4H), 7.47 (d, 2H), 7.31 (t, 2H), 7.00 (t, 1H), 2.93 (m, 1H), 2.77 (d, 2H), 2.46 (s, 3H), 2.14 (m, 2H), 1.85 (m, 3H), 1.54 (m, 2H), 1.24 (m, 2H); MS: m/z 474.2 (M+1).

### Example 385

### 2,6-Difluoro-N-(4-(2-(4-((5-methyl-1,3,4-oxadiazol-2-yl)methyl)cyclohexyl)thiazol-5-yl)phenyl)benzamide

The compound of example 385 was prepared analogous to the compound of example 14 by reaction of the compound of example 378 with 2,6-difluoro benzoyl chloride. ¹H NMR (DMSO-d₆, 300 MHz): δ 10.91 (s, 1H), 7.99 (s, 1H), 7.74 (d, 2H), 7.62 (d, 2H), 7.59 (m, 1H), 7.27 (m, 2H), 2.96 (m, 1H), 2.75 (d, 2H), 2.45 (s, 3H), 2.12 (m, 2H), 1.83 (m, 3H), 1.56 (m, 2H), 1.27 (m, 2H); MS: m/z 495.2 (M+1).

### Example 386

### 2-(4-(5-(4-Nitrophenyl)thiazol-2-yl)cyclohexyl)acetic acid

To a solution of the compound of example 353 (1.8 g, 4.81 mmol) in methanol (10 mL) and THF (10 mL) was added sodium hydroxide (0.961 g, 24.03 mmol) and the reaction mixture was stirred for 16 h at room temperature. After completion of the reaction, the reaction mixture was acidified with dilute HCl to obtain a solid, which was filtered, washed with water and dried to afford the title compound. Yield: 1.25 g (67%); ¹H NMR (DMSO-d₆, 300 MHz): δ 12.04 (s, 1H), 8.32 (s, 1H), 8.26 (d, 2H), 7.91 (d, 2H), 3.00 (m, 1H), 2.14 (d, 2H), 2.09 (m, 2H), 1.84 (m, 2H), 1.72 (m, 1H), 1.58 (m, 2H), 1.21 (m, 2H); MS: m/z 347.1 (M+1).

### Example 387

### (E)-N-(1-(Hydroxyimino)ethyl)-2-(4-(5-(4-nitrophenyl)thiazol-2-yl)cyclohexyl)acetamide

To a solution of the compound of example 386 (1.30 g, 3.75 mmol) in dichloroethane (10 mL) was added oxalyl chloride (8.21 mL, 94 mmol) and the reaction mixture was stirred for 16 h at room temperature. The solvent was removed, toluene was added and evaporated to remove the unreacted oxalyl chloride. The resulting solid was taken in dioxane, N-hydroxyacetamidine (1.668 g, 22.52 mmol) was added and the reaction mixture was stirred for 16 h at room temperature. After completion of the reaction, the compound was adsorbed on silica and purified using flash column chromatography (silica gel, 20% ethyl acetate in chloroform) to afford the title compound. Yield: 850 mg (56° A)); ¹H NMR (CDCl₃, 300 MHz): δ 8.26 (d, 2H), 7.97 (s, 1H), 7.69 (d, 2H), 4.73 (bs, 2H), 3.02 (m, 1H), 2.36 (d, 2H), 2.26 (m, 2H), 1.99 (m, 6H), 1.70 (m, 2H), 1.29 (m, 2H); MS: m/z 403.1 (M+1).

### Example 388

### 3-Methyl-5-((4-(5-(4-nitrophenyl)thiazol-2-yl)cyclohexyl)methyl)-1,2,4-oxadiazole

The compound of example 387 (800 mg, 1.988 mmol) was dissolved in DMF (20 mL) and stirred at 120° C. under microwave irradiation for 3 h. After completion of the reaction, the resulting mixture was adsorbed onto silica and purified using flash column chromatography (silica gel, 20-30° A) ethyl acetate in chloroform) to afford the title compound. Yield: 700 mg (91%); ¹H NMR (DMSO-d₆, 300 MHz): δ 8.31 (s, 1H), 8.26 (d, 2H), 7.91 (d, 2H), 3.02 (m, 1H), 2.84 (d, 2H), 2.30 (s, 3H), 2.14 (m, 2H), 1.83 (m, 3H), 1.55 (m, 2H), 1.25 (m, 2H); MS: m/z 385.1 (M+1).

### Example 389

### 4-(2-(4-((3-Methyl-1,2,4-oxadiazol-5-yl)methyl)cyclohexyl)thiazol-5-yl)aniline

To a solution of the compound of example 388 (750 mg, 1.951 mmol) in dioxane (5 mL) at 80° C. was added a hot solution of sodium sulfide (381 mg, 4.88 mmol) in water (5 mL) and the reaction mixture was stirred for 1 h at 80-85° C. After completion of the reaction, water was added and the product was extracted using ethyl acetate. This crude product was further purified by flash column chromatography (silica gel, 23-35° A) ethyl acetate in chloroform) to afford the title compound. Yield: 680 mg (98° A)); ¹H NMR (DMSO-d₆, 300 MHz): δ 7.70 (s, 1H), 7.24 (d, 2H), 6.56 (d, 2H), 5.34 (s, 2H), 2.89 (m, 1H), 2.82 (d, 2H), 2.29 (s, 3H), 2.08 (m, 2H), 1.81 (m, 3H), 1.54 (m, 2H), 1.26 (m, 2H); MS: m/z 355.2 (M+1).

### Example 390

### 1-(2-Chlorophenyl)-3-(4-(2-(4-((3-methyl-1,2,4-oxadiazol-5-yl)methyl)cyclohexyl)thiazol-5-yl)phenyl)urea

The compound of example 390 was prepared analogous to the compound of example 6 by reaction of the compound of example 389 with 2-chloro-1-isocyanatobenzene.

Yield: 82%; ¹H NMR (DMSO-d₆, 300 MHz): δ 9.56 (s, 1H), 8.34 (s, 1H), 8.18 (dd, 1H), 7.95 (s, 1H), 7.57 (m, 4H), 7.48 (dd, 1H), 7.33 (m, 1H), 7.07 (m, 1H), 2.96 (m, 1H), 2.85 (d, 2H), 2.32 (s, 3H), 2.13 (m, 2H), 1.84 (m, 3H), 1.59 (m, 2H), 1.30 (m, 2H); MS: m/z 508.1 (M+1).

### Example 391

### 1-(2-Fluorophenyl)-3-(4-(2-(4-((3-methyl-1,2,4-oxadiazol-5-yl)methyl)cyclohexyl)thiazol-5-yl)phenyl)urea

The compound of example 391 was prepared analogous to the compound of example 6 by reaction of the compound of example 389 with 2-fluoro-1-isocyanatobenzene.

Yield: 81%; ¹H NMR (DMSO-d₆, 300 MHz): δ 9.22 (s, 1H), 8.58 (s, 1H), 8.17 (m, 1H), 7.95 (s, 1H), 7.56 (m, 4H), 7.27 (m, 1H), 7.17 (t, 1H), 7.05 (m, 1H), 2.96 (m, 1H), 2.85 (d, 2H), 2.32 (s, 3H), 2.13 (m, 2H), 1.88 (m, 3H), 1.58 (m, 2H), 1.29 (m, 2H); MS: m/z 492.1 (M+1).

### Example 392

### 1-(3,5-Difluorophenyl)-3-(4-(2-(4-((3-methyl-1,2,4-oxadiazol-5-yl)methyl)cyclohexyl)thiazol-5-yl)phenyl)urea

The compound of example 392 was prepared analogous to the compound of example 6 by reaction of the compound of example 389 with 3,5-difluoro-1-isocyanatobenzene.

Yield: 80%; ¹H NMR (DMSO-d₆, 300 MHz): δ 9.12 (s, 1H), 9.01 (s, 1H), 7.95 (s, 1H), 7.56 (d, 2H), 7.52 (d, 2H), 7.23 (m, 2H), 6.84 (m, 1H), 2.96 (m, 1H), 2.85 (d, 2H), 2.32 (s, 3H), 2.13 (m, 2H), 1.84 (m, 3H), 1.57 (m, 2H), 1.29 (m, 2H); MS: m/z 510.1 (M+1).

### Example 393

### 1-(4-(2-(4-((3-Methyl-1,2,4-oxadiazol-5-yl)methyl)cyclohexyl)thiazol-5-yl)phenyl)-3-(2,4,5-trifluorophenyl)urea

The compound of example 393 was prepared analogous to the compound of example by reaction of the compound of example 389 with 2,4,5-trifluoro-1-isocyanatobenzene. Yield: 64%; ¹H NMR (DMSO-d₆, 300 MHz): δ 9.23 (s, 1H), 8.75 (s, 1H), 8.24 (m, 1H), 7.95 (s, 1H), 7.69 (m, 1H), 7.57 (d, 2H), 7.51 (d, 2H), 2.96 (m, 1H), 2.85 (d, 2H), 2.32 (s, 3H), 2.13 (m, 2H), 1.88 (m, 3H), 1.58 (m, 2H), 1.29 (m, 2H); MS: m/z 528.1 (M+1).

### Example 394

### 1-(2,4-Difluorophenyl)-3-(4-(2-(4-((3-methyl-1,2,4-oxadiazol-5-yl)methyl)cyclohexyl)thiazol-5-yl)phenyl)urea

The compound of example 394 was prepared analogous to the compound of example 6 by reaction of the compound of example 389 with 2,4-difluoro-1-isocyanatobenzene.

Yield: 88%; ¹H NMR (DMSO-d₆, 300 MHz): δ 9.17 (s, 1H), 8.53 (s, 1H), 8.10 (m, 1H), 7.95 (s, 1H), 7.55 (d, 2H), 7.51 (d, 2H), 7.34 (m, 1H), 7.07 (m, 1H), 2.92 (m, 1H), 2.85 (d, 2H), 2.32 (s, 3H), 2.13 (m, 2H), 1.86 (m, 3H), 1.55 (m, 2H), 1.27 (m, 2H); MS: m/z 510.2 (M+1).

### Example 395

### 1-(4-(2-(4-((3-Methyl-1,2,4-oxadiazol-5-yl)methyl)cyclohexyl)thiazol-5-yl)phenyl)-3-phenylurea

The compound of example 395 was prepared analogous to the compound of example 6 by reaction of the compound of example 389 with phenylisocyanate. Yield: 58%; ¹H NMR (DMSO-d₆, 300 MHz): δ 8.85 (s, 1H), 8.72 (s, 1H), 7.94 (s, 1H), 7.55 (m, 4H), 7.47 (d, 2H), 7.31 (t, 2H), 7.00 (t, 1H), 2.92 (m, 1H), 2.85 (d, 2H), 2.32 (s, 3H), 2.13 (m, 2H), 1.84 (m, 3H), 1.54 (m, 2H), 1.26 (m, 2H); MS: m/z 474.2 (M+1).

### Example 396

### 2,6-Difluoro-N-(4-(2-(4-((3-methyl-1,2,4-oxadiazol-5-yl)methyl)cyclohexyl)thiazol-5-yl)phenyl)benzamide

The compound of example 396 was prepared analogous to the compound of example 14 by reaction of the compound of example 389 with 2,6-difluorobenzoyl chloride. Yield: 70%; ¹H NMR (DMSO-d₆, 300 MHz): δ 10.93 (s, 1H), 8.01 (s, 1H), 7.75 (d, 2H), 7.64 (d, 2H), 7.60 (m, 1H), 7.28 (t, 2H), 2.96 (m, 1H), 2.85 (d, 2H), 2.32 (s, 3H), 2.13 (m, 2H), 1.86 (m, 3H), 1.56 (m, 2H), 1.28 (m, 2H); MS: m/z 495.1 (M+1).

### Example 397

### 2-Chloro-N-(4-(2-(4-((3-methyl-1,2,4-oxadiazol-5-yl)methyl)cyclohexyl)thiazol-5-yl)phenyl)benzamide

The compound of example 397 was prepared analogous to the compound of example 14 by reaction of the compound of example 389 with 2-chlorobenzoyl chloride. Yield: 58%; ¹H NMR (DMSO-d₆, 300 MHz): δ 10.64 (s, 1H), 8.01 (s, 1H), 7.79 (d, 2H), 7.62 (d, 2H), 7.59 (m, 2H), 7.50 (m, 2H), 2.98 (m, 1H), 2.85 (d, 2H), 2.32 (s, 3H), 2.14 (m, 2H), 1.84 (m, 3H), 1.58 (m, 2H), 1.30 (m, 2H); MS: m/z 493.1 (M+1).

### Example 398

### 3,5-Difluoro-N-(4-(2-(4-((3-methyl-1,2,4-oxadiazol-5-yl)methyl)cyclohexyl)thiazol-5-yl)phenyl)benzamide

The compound of example 398 was prepared analogous to the compound of example 14 by reaction of the compound of example 389 with 3,5-difluorobenzoyl chloride.

Yield: 62%; ¹H NMR (DMSO-d₆, 300 MHz): δ 10.47 (s, 1H), 8.01 (s, 1H), 7.84 (d, 2H), 7.70 (m, 2H), 7.65 (d, 2H), 7.58 (m, 1H), 2.94 (m, 1H), 2.85 (d, 2H), 2.32 (s, 3H), 2.14 (m, 2H), 1.84 (m, 3H), 1.55 (m, 2H), 1.26 (m, 2H); MS: m/z 495.2 (M+1).

### Example 399

### N-Acetyl-2-(4-(5-(4-aminophenyl)thiazol-2-yl)cyclohexyl)acetamide

To a solution of the compound of example 388 (800 mg, 2.081 mmol) in ethanol (10 mL), water (5 mL) and THF (5 mL), were added iron (581 mg, 10.40 mmol) and ammonium chloride (557 mg, 10.40 mmol) and the reaction mixture was stirred at 85° C. for 3 h. After completion of the reaction, the reaction mixture was cooled to room temperature and the solid obtained was filtered through Celite® followed by concentration of the organic solvent. Saturated NaHCO₃ solution was added and the compound was extracted using ethyl acetate. The organic layer was concentrated to obtain the crude compound. The crude compound was purified using flash column chromatography (silica gel, 15% ethyl acetate in chloroform) to afford the title compound. Yield: 235 mg (31%); ¹H NMR (DMSO-d₆, 300 MHz): δ 10.58 (s, 1H), 7.71 (s, 1H), 7.25 (d, 2H), 6.57 (d, 2H), 5.35 (s, 2H), 2.89 (m, 1H), 2.34 (d, 2H), 2.15 (s, 3H), 2.08 (m, 2H), 1.80 (m, 3H), 1.51 (m, 2H), 1.81 (m, 2H); MS: m/z 358.2 (M+1).

### Example 400

### N-Acetyl-2-(4-(5-(4-(3-(2-chlorophenyl)ureido)phenyl)thiazol-2-yl)cyclohexyl)acetamide

The compound of example 400 was prepared analogous to the compound of example 6 by reaction of the compound of example 399 with 2-chlorophenyl isocyanate. Yield: 59%; ¹H NMR (DMSO-d₆, 300 MHz): δ 10.59 (s, 1H), 9.55 (s, 1H), 8.32 (s, 1H), 8.16 (d, 1H), 7.93 (s, 1H), 7.56 (d, 2H), 7.51 (d, 2H), 7.46 (dd, 1H), 7.32 (t, 1H), 7.05 (m, 1H), 2.94 (m, 1H), 2.35 (d, 2H), 2.16 (s, 3H), 2.11 (m, 2H), 1.82 (m, 3H), 1.55 (m, 2H), 1.18 (m, 2H); MS: m/z 511.2 (M+1).

### Example 401

### N-Acetyl-2-(4-(5-(4-(3-(2,4-difluorophenyl)ureido)phenyl)thiazol-2-yl)cyclohexyl)acetamide

The compound of example 401 was prepared analogous to the compound of example 6 by reaction of the compound of example 399 with 2,4-difluoro phenyl isocyanate.

Yield: 44%; ¹H NMR (DMSO-d₆, 300 MHz): δ 10.59 (s, 1H), 9.15 (s, 1H), 8.52 (s, 1H), 8.10 (m, 1H), 7.93 (s, 1H), 7.54 (d, 2H), 7.49 (d, 2H), 7.34 (m, 1H), 7.07 (m, 1H), 2.94 (m, 1H), 2.35 (d, 2H), 2.16 (s, 3H), 2.10 (m, 2H), 1.81 (m, 3H), 1.54 (m, 2H), 1.18 (m, 2H); MS: m/z 513.2 (M+1).

### Example 402

### N-Acetyl-2-(4-(5-(4-(3-(2,4,5-trifluorophenyl)ureido)phenyl)thiazol-2-yl)cyclohexyl)acetamide

The compound of example 402 was prepared analogous to the compound of example 6 by reaction of the compound of example 399 with 2,4,5-trifluorophenyl isocyanate.

Yield: 44%; ¹H NMR (DMSO-d₆, 300 MHz): δ 10.59 (s, 1H), 9.21 (s, 1H), 8.73 (s, 1H), 8.22 (m, 1H), 7.93 (s, 1H), 7.67 (m, 1H), 7.55 (d, 2H), 7.49 (d, 2H), 2.94 (m, 1H), 2.35 (d, 2H), 2.15 (s, 3H), 2.10 (m, 2H), 1.81 (m, 3H), 1.54 (m, 2H), 1.18 (m, 2H); MS: m/z 531.2 (M+1).

### Example 403

### N-(4-(2-(4-(2-Acetamido-2-oxoethyl)cyclohexyl)thiazol-5-yl)phenyl)-2,6-difluoro benzamide

The compound of example 403 was prepared analogous to the compound of example 14 by reaction of the compound of example 399 with 2,6-difluorobenzoyl chloride. Yield: 47%; ¹H NMR (DMSO-d₆, 300 MHz): δ 10.91 (s, 1H), 10.59 (s, 1H), 7.99 (s, 1H), 7.74 (d, 2H), 7.63 (d, 2H), 7.59 (m, 1H), 7.27 (t, 2H), 2.96 (m, 1H), 2.35 (d, 2H), 2.16 (s, 3H), 2.11 (m, 2H), 1.82 (m, 3H), 1.55 (m, 2H), 1.19 (m, 2H); MS: m/z 498.2 (M+1).

### Example 404

### 1-(2-Chlorophenyl)-3-(4-(2-(4-(2-hydroxypropan-2-yl)cyclohexyl)thiazol-5-yl)phenyl)urea

To a solution of the compound of example 187 (200 mg, 0.426 mmol) in toluene (10 mL) was added methyl magnesium bromide (507 mg, 4.26 mmol) at 5° C. The reaction mixture was stirred at room temperature for 16 h. After completion of the reaction, water was added to the reaction mixture followed by an extraction with ethyl acetate. The organic layer was washed with water and concentrated. The crude compound was purified using flash column chromatography (silica gel, 25% ethyl acetate in chloroform) to afford the title compound. Yield: 87 mg (47%); ¹H NMR (DMSO-d₆, 300 MHz): δ 9.55 (s, 1H), 8.32 (s, 1H), 8.16 (dd, 1H), 7.93 (s, 1H), 7.56 (d, 2H), 7.31 (d, 2H), 7.46 (dd, 1H), 7.32 (m, 1H), 7.05 (m, 1H), 4.07 (s, 1H), 2.90 (m, 1H), 2.16 (m, 2H), 1.91 (m, 2H), 1.49 (m, 2H), 1.25 (m, 3H), 1.04 (s, 6H); MS: m/z 470.2 (M+1).

### Example 405

### 1-(3,5-Difluorophenyl)-3-(4-(2-(4-(2-hydroxypropan-2-yl)cyclohexyl)thiazol-5-yl) phenyl)urea

The compound of example 405 was prepared analogous to the compound of example 404 by reaction of compound of example 182 with methyl magnesium bromide. Yield: 34%; ¹H NMR (DMSO-d₆, 300 MHz): δ 9.10 (s, 1H), 8.99 (s, 1H), 7.92 (s, 1H), 7.55 (d, 2H), 7.50 (d, 2H), 7.21 (m, 2H), 6.82 (m, 1H), 4.07 (s, 1H), 2.89 (m, 1H), 2.16 (m, 2H), 1.91 (m, 2H), 1.49 (m, 2H), 1.25 (m, 3H), 1.04 (s, 6H); MS: m/z 472.2 (M+1).

### Example 406

### 1-(2,4-Difluorophenyl)-3-(4-(2-(4-(2-hydroxypropan-2-yl)cyclohexyl)thiazol-5-yl)phenyl)urea

The compound of example 406 was prepared analogous to the compound of example 404 by reaction of compound of example 137 with methyl magnesium bromide. Yield: 34%; 1H NMR (DMSO-d₆, 300 MHz): δ 9.24 (s, 1H), 8.60 (s, 1H), 8.08 (m, 1H), 7.94 (s, 1H), 7.56 (d, 2H), 7.49 (d, 2H), 7.35 (m, 1H), 7.05 (m, 1H), 4.08 (s, 1H), 2.92 (m, 1H), 2.17 (m, 2H), 1.93 (m, 2H), 1.50 (m, 2H), 1.26 (m, 3H), 1.05 (s, 6H); MS: m/z 472.2 (M+1).

### Example 407

### 1-(2,4-Difluorophenyl)-3-(4-(2-(4-(2-hydroxy-2-methylpropyl)cyclohexyl)thiazol-5-yl)phenyl)urea

The compound of example 407 was prepared analogous to the compound of example 404 by reaction of compound of example 361 with methyl magnesium bromide. Yield: 34%; ¹H NMR (DMSO-d₆, 300 MHz): δ 9.15 (s, 1H), 8.52 (s, 1H), 8.07 (m, 1H), 7.92 (s, 1H), 7.54 (d, 2H), 7.49 (d, 2H), 7.34 (m, 1H), 7.03 (m, 1H), 4.04 (s, 1H), 2.88 (m, 1H), 2.07 (m, 2H), 1.92 (m, 2H), 1.54 (m, 3H), 1.29 (d, 2H), 1.15 (m, 2H), 1.09 (s, 6H); MS: m/z 486.2 (M+1).

### Example 408

### 1-(3,5-Difluorophenyl)-3-(4-(2-(4-(2-hydroxy-2-methylpropyl)cyclohexyl)thiazol-5-yl)phenyl)urea

The compound of example 408 was prepared analogous to the compound of example 404 by reaction of compound of example 355 with methyl magnesium bromide. Yield: 34%; ¹H NMR (DMSO-d₆, 300 MHz): δ 9.20 (s, 1H), 8.72 (s, 1H), 8.22 (m, 1H), 7.92 (s, 1H), 7.67 (m, 2H), 7.55 (d, 2H), 7.49 (d, 2H), 4.04 (s, 1H), 2.90 (m, 1H), 2.07 (m, 2H), 1.92 (m, 2H), 1.54 (m, 3H), 1.29 (d, 2H), 1.14 (m, 2H), 1.09 (s, 6H); MS: m/z 486.2 (M+1).

### Example 409

### 1-(4-(2-(4-(2-Hydroxy-2-methylpropyl)cyclohexyl)thiazol-5-yl-phenyl)-3-(2,4,5-trifluorophenyl)urea

The compound of example 409 was prepared analogous to the compound of example 404 by reaction of compound of example 357 with methyl magnesium bromide. Yield: 34%; ¹H NMR (DMSO-d₆, 300 MHz): δ 9.22 (s, 1H), 8.75 (s, 1H), 8.21 (m, 1H), 7.94 (s, 1H), 7.66 (m, 1H), 7.57 (d, 2H), 7.51 (d, 2H), 4.06 (s, 1H), 2.92 (m, 1H), 2.09 (m, 2H), 1.94 (m, 2H), 1.56 (m, 3H), 1.30 (d, 2H), 1.16 (m, 2H), 1.01 (s, 6H); MS: m/z 504.2 (M+1).

### Example 410

### 1-(3,5-Difluorophenyl)-3-(4-(2-(4-(2-hydrazinyl-2-oxoethyl)cyclohexyl) thiazol-5-yl)phenyl)urea

A mixture of the compound of example 355 (200 mg, 0.400 mmol) and hydrazine hydrate (1.257 mL, 40.0 mmol) was stirred at 80° C. for 15 min followed by addition of ethanol (5 mL). This reaction mixture was then stirred at 80° C. for an additional 4-5 h. After completion of the reaction, the reaction mixture was cooled to room temperature. The precipitated solid was filtered and dried to afford the title compound. Yield: 122 mg (61%); ¹H NMR (DMSO-d₆, 300 MHz): δ 9.91 (d, 1H), 9.11 (s, 1H), 9.00 (s, 1H), 7.93 (s, 1H), 7.54 (d, 2H), 7.50 (d, 2H), 7.18 (d, 2H), 6.78 (m, 1H), 2.89 (m, 1H), 2.13 (m, 2H), 1.89 (d, 2H), 1.82 (m, 5H), 1.50 (m, 2H), 1.15 (m, 2H); MS: m/z 486.6 (M+1).

### Example 411

### N′-Acetyl-2-(4-(5-(4-nitrophenyl)thiazol-2-yl)cyclohexyl)acetohydrazide

To a solution of the compound of example 386 (300 mg, 0.866 mmol) in dichloroethane (10 mL) was added oxalyl chloride (2.7 g, 21.65 mmol) and the reaction mixture was stirred for 32 h at room temperature. The solvent was removed, toluene was added and the reaction mixture was concentrated to remove the unreacted oxalyl chloride. The resulting solid was taken in dioxane (10 mL), acetic hydrazide (64.2 mg, 0.866 mmol) was added and reaction mixture was stirred at room temperature for 16 h. Following the completion of the reaction, the compound was adsorbed onto silica and purified using flash column chromatography (silica gel, 5% methanol in chloroform) to afford the title compound. Yield: 180 mg (48%); ¹H NMR (DMSO-d₆, 300 MHz): δ 9.71 (s, 1H), 9.69 (s, 1H), 8.32 (s, 1H), 8.26 (d, 2H), 7.91 (d, 2H), 3.01 (m, 2H), 2.13 (m, 1H), 2.04 (d, 2H), 1.85 (m, 6H), 1.85 (m, 2H), 1.18 (m, 2H); MS: m/z 403.1 (M+1).

### Example 412

### 2-Methyl-5-((4-(5-(4-nitrophenyl)thiazol-2-yl)cyclohexyl)methyl)-1,3,4-thiadiazole

To a solution of the compound of example 411 (500 mg, 1.242 mmol) in xylene (10 mL) was added Lawesson's Reagent (502 mg, 1.242 mmol) and the reaction mixture was stirred at 130° C. for 3 h. After completion of the reaction, water was added and the reaction mixture was extracted with ethyl acetate. The combined organic layers were washed with water, concentrated and purified using flash column chromatography (silica gel, 20% ethyl acetate in chloroform) to afford the title compound. Yield: 350 mg (43%); ¹H NMR (DMSO-d₆, 300 MHz): δ 8.34 (s, 1H), 8.28 (d, 2H), 7.93 (d, 2H), 3.00 (m, 2H), 2.77 (m, 1H), 2.69 (s, 3H), 2.46 (m, 1H), 2.15 (m, 2H), 1.85 (m, 2H), 1.55 (m, 2H), 1.30 (m, 2H); MS: m/z 401.1 (M+1).

### Example 413

### 4-(2-(4-((5-Methyl-1,3,4-thiadiazol-2-yl)methyl)cyclohexyl)thiazol-5-yl)aniline

The compound of example 413 was prepared analogous to the compound of example 378 by reduction of compound of example 412. Yield: 150 mg (35%); ¹H NMR (DMSO-d₆, 300 MHz): δ 7.72 (s, 1H), 7.26 (d, 2H), 6.58 (d, 2H), 5.36 (s, 2H), 2.98 (d, 2H), 2.92 (m, 1H), 2.68 (s, 3H), 2.11 (m, 2H), 1.83 (m, 3H), 1.49 (m, 2H), 1.22 (m, 2H); MS: m/z 371.1 (M+1).

### Example 414

### 1-(4-(2-(4-((5-Methyl-1,3,4-thiadiazol-2-yl)methyl)cyclohexyl)thiazol-5-yl)phenyl)-3-(2,4,5-trifluorophenyl)urea

The compound of example 414 was prepared analogous to the compound of example by reaction of the compound of example 413 with 2,4,5-trifluoro-1-isocyanatobenzene. Yield: 47%; ¹H NMR (DMSO-d₆, 300 MHz): δ 9.23 (s, 1H), 8.75 (s, 1H), 8.24 (m, 1H), 7.95 (s, 1H), 7.69 (m, 1H), 7.57 (d, 2H), 7.51 (d, 2H), 2.99 (d, 2H), 2.93 (m, 1H), 2.69 (s, 3H), 2.13 (m, 2H), 1.84 (m, 3H), 1.52 (m, 2H), 1.27 (m, 2H); MS: m/z 544.1 (M+1).

### Example 415

### t-Butyl 4-(4-(4-nitrophenyl)thiazol-2-yl)piperidine-1-carboxylate

A solution of 2-bromo-1-(4-nitrophenyl)ethanone (0.5 g, 2.049 mmol) and tert-butyl 4-carbamothioylpiperidine-1-carboxylate (0.601 g, 2.459 mmol) in EtOH (10 mL) was refluxed for 4 h under stirring. After completion of reaction, solvent was removed and the crude material obtained was purified by column chromatography (silica gel, 30% ethyl acetate in petroleum ether). Yield: 69%; ¹H NMR (DMSO-d₆, 300 MHz): δ 8.37 (s, 1H), 8.32 (d, 2H), 8.23 (d, 2H), 4.05 (m, 1H), 3.29 (m, 2H), 2.92 (m, 2H), 2.01 (m, 2H), 1.66 (m, 2H), 1.41 (s, 9H); MS: m/z 391 (M+1).

### Example 416

### 4-(4-Nitrophenyl)-2-(piperidin-4-yl)thiazole hydrochloride

To a solution of the compound of example 415 (0.8 g, 2.054 mmol) in ethyl acetate was added followed by HCl in ethyl acetate and the reaction mixture was stirred at room temperature for 16 h. After completion of reaction, solvent was removed and the residue obtained was triturated with diethyl ether. The solid obtained was filtered and dried to afford the title compound. Yield: 75%; ¹H NMR (DMSO-d₆, 300 MHz): δ 9.10 (s, 1H), 8.40 (s, 1H), 8.31 (d, 2H), 8.21 (d, 2H), 3.46 (m, 3H), 3.08 (m, 2H), 2.25 (m, 2H), 2.03 (m, 2H); MS: m/z 290 (M+1).

### Example 417

### Ethyl 2-(4-(4-(4-nitrophenyl)thiazol-2-yl)piperidin-1-yl)acetate

To a solution of the compound of example 416 (0.8 g, 2.161 mmol) in toluene (5 mL) was added triethylamine (0.903 mL, 6.48 mmol) and ethyl 2-chloroacetate (0.397 g, 3.24 mmol) and the reaction mixture was stirred at 112° C. for 16 h. After completion of reaction, ethyl acetate was added to it and the resulting mixture was washed with water and brine, dried over sodium sulfate and concentrated. The material obtained was purified by column chromatography (silica gel, 30% ethyl acetate in petroleum ether);

Yield: 62%; ¹H NMR (DMSO-d₆, 300 MHz): δ 8.34 (s, 1H), 8.29 (d, 2H), 8.20 (d, 2H), 4.10 (q, 2H), 3.29 (s, 2H), 3.02 (m, 1H), 2.92 (m, 2H), 2.37 (m, 2H), 2.06 (m, 2H), 1.76 (m, 2H), 1.19 (t, 3H); MS: m/z 376 (M+1).

### Example 418

### Ethyl 2-(4-(4-(4-aminophenyl)thiazol-2-yl)piperidin-1-yl)acetate

The compound of example 418 was prepared analogous to the compound of example 378 by reduction of compound of example 417. Yield: 82%; ¹H NMR (DMSO-d₆, 300 MHz): δ 7.58 (d, 2H), 7.50 (s, 1H), 6.56 (d, 2H), 5.24 (s, 2H), 4.10 (q, 2H), 3.21 (s, 2H), 2.92 (m, 3H), 2.34 (m, 2H), 2.02 (m, 2H), 1.71 (m, 2H), 1.17 (t, 3H); MS: m/z 346 (M+1).

### Example 419

### Ethyl 2-(4-(4-(4-(3-(2-fluorophenyl)ureido)phenyl)thiazol-2-yl)piperidin-1-yl)acetate

The compound of example 419 was prepared analogous to the compound of example 6 by reaction of the compound of example 418 with 2-fluoro-1-isocyanatobenzene.

Yield: 86%; ¹H NMR (DMSO-d₆, 300 MHz): δ 9.17 (s, 1H), 8.55 (s, 1H), 8.14 (t, 1H), 7.86 (d, 2H), 7.81 (s, 1H), 7.50 (d, 2H), 7.22 (m, 1H), 7.12 (t, 1H), 6.99 (m, 1H), 4.10 (q, 2H), 3.29 (s, 2H), 2.97 (m, 3H), 2.36 (m, 2H), 2.05 (m, 2H), 1.73 (m, 2H), 1.20 (m, 3H); MS: m/z 483 (M+1).

### Example 420

### 2-(4-(4-(4-(3-(2-Fluorophenyl)ureido)phenyl)thiazol-2-yl)piperidin-1-yl)acetic acid

The compound of example 420 was prepared analogous to the compound of example 7 by hydrolysis of the compound of example 419. Yield: 89%; ¹H NMR (DMSO-d₆, 300 MHz): δ 9.37 (s, 1H), 8.72 (s, 1H), 8.14 (t, 1H), 7.86 (d, 2H), 7.84 (s, 1H), 7.52 (d, 2H), 7.25 (m, 1H), 7.14 (t, 1H), 7.02 (m, 1H), 3.33 (s, 2H), 3.25 (m, 2H), 3.14 (m, 1H), 2.74 (m, 2H), 2.16 (m, 2H), 1.96 (m, 2H); MS: m/z 455 (M+1).

### Example 421

### Ethyl 2-(4-(4-(4-(3-(2-chlorophenyl)ureido)phenyl)thiazol-2-yl)piperidin-1-yl)acetate

The compound of example 421 was prepared analogous to the compound of example 6 by reaction of the compound of example 418 with 2-chloro-1-isocyanatobenzene.

Yield: 87%; ¹H NMR (DMSO-d₆, 300 MHz): δ 9.51 (s, 1H), 8.31 (s, 1H), 8.16 (dd, 1H), 7.87 (d, 2H), 7.81 (s, 1H), 7.51 (d, 2H), 7.45 (dd, 1H), 7.28 (t, 1H), 7.01 (m, 1H), 4.10 (q, 2H), 3.22 (s, 2H), 2.92 (m, 1H), 2.88 (m, 2H), 2.36 (m, 2H), 2.05 (m, 2H), 1.74 (m, 2H), 1.20 (m, 3H); MS: m/z 499 (M+1).

### Example 422

### 2-(4-(4-(4-(3-(2-Chlorophenyl)ureido)phenyl)thiazol-2-yl)piperidin-1-yl)acetic acid

The compound of example 422 was prepared analogous to the compound of example 7 by hydrolysis of the compound of example 421. Yield: 81%; ¹H NMR (DMSO-d₆, 300 MHz): δ 9.80 (s, 1H), 8.45 (s, 1H), 8.14 (d, 1H), 7.88 (d, 3H), 7.54 (d, 2H), 7.45 (d, 1H), 7.30 (t, 1H), 7.04 (t, 1H), 3.99 (s, 2H), 3.52 (m, 2H), 3.18 (m, 2H), 3.14 (m, 1H), 2.29 (m, 2H), 2.10 (m, 2H); MS: m/z 471 (M−1).

### Example 423

### t-Butyl 4-((2-(4-nitrophenyl)-2-oxoethyl)carbamoyl)piperidine-1-carboxylate

To 1-(tert-butoxycarbonyl)piperidine-4-carboxylic acid (2 g, 8.72 mmol) in DMF (20 mL) were added HATU (3.65 g, 9.60 mmol) and the reaction mixture was stirred for 15 min at room temperature. 2-amino-1-(4-nitrophenyl)ethanone hydrochloride (2.268 g, 10.47 mmol) was added to the reaction mixture at room temperature. After 10 min of stirring, DIPEA (4.57 mL, 26.2 mmol) was added slowly. After completion of the reaction, the reaction mixture was cooled to room temperature, water was added and the resulting mixture was extracted with ethyl acetate. The organic layer was passed through Celite® to remove insoluble solid and washed with 3N HCl, NaHCO₃ and water. The solvent was removed to yield a solid, which was purified by column chromatography (silica gel, 30% ethyl acetate in petroleum ether) to afford the title compound. Yield: 60%; ¹H NMR (DMSO-d₆, 300 MHz): δ 8.33 (d, 2H), 8.17 (d, 2H), 4.60 (d, 1H), 3.91 (m, 2H), 2.70 (m, 3H), 2.41 (m, 3H), 1.67 (m, 2H), 1.41 (m, 9H); MS: m/z 392 (M+1).

### Example 424

### t-Butyl 4-(5-(4-nitrophenyl)thiazol-2-yl)piperidine-1-carboxylate

To a solution of the compound of example 423 (1 g, 2.55 mmol) in dioxane (20 mL) was added Lawesson's reagent (1.137 g, 2.81 mmol) and the reaction mixture was stirred at 55° C. for 3 h. After completion of the reaction, the reaction mixture was cooled to room temperature and basified with aq. NaHCO₃ followed by extraction with ethyl acetate. The organic layer was washed with water and brine solution and the solvent was evaporated to yield a solid, which was purified by column chromatography (silica gel, 30% ethyl acetate in petroleum ether) to afford the title compound. Yield: 56%; ¹H NMR (DMSO-d₆, 300 MHz): δ 8.35 (s, 1H), 8.26 (d, 2H), 7.92 (d, 2H), 4.01 (d, 2H), 3.26 (m, 1H), 2.86 (m, 2H), 2.06 (m, 2H), 1.59 (m, 2H), 1.39 (m, 9H); MS: m/z 390 (M+1).

### Example 425

### 5-(4-Nitrophenyl)-2-(piperidin-4-yl)thiazole hydrochloride

To a solution of the compound of example 424 (0.6 g, 1.541 mmol) in THF (25 mL) and ethyl acetate (25 mL), was added hydrochloric acid in ethyl acetate (10 mL) and stirred at room temperature for 16 h. After completion of reaction, the reaction mixture was concentrated to yield a solid, which was triturated with diethyl ether and the solid obtained was filtered and dried to afford the title compound. Yield: 90%; ¹H NMR

(DMSO-d₆, 300 MHz): δ 8.90 (s, 1H), 8.38 (s, 1H), 8.27 (d, 2H), 7.93 (d, 2H), 3.44 (m, 3H), 3.07 (m, 2H), 2.22 (m, 2H), 2.00 (m, 2H); MS: m/z 290 (M+1).

### Example 426

### Ethyl 2-(4-(5-(4-nitrophenyl)thiazol-2-yl)piperidin-1-yl)acetate

The compound of example 426 was prepared analogous to the compound of example 417 by reaction of the compound of example 425 with ethyl 2-chloroacetate. Yield: 52%; ¹H NMR (DMSO-d₆, 300 MHz): δ 8.33 (s, 1H), 8.25 (d, 2H), 7.19 (d, 2H), 4.01 (d, 2H), 3.22 (s, 2H), 3.02 (m, 1H), 2.91 (m, 2H), 2.36 (m, 2H), 2.04 (m, 2H), 1.77 (m, 2H), 1.19 (t, 3H); MS: m/z 376 (M+1).

### Example 427

### Ethyl 2-(4-(5-(4-aminophenyl)thiazol-2-yl)piperidin-1-yl)acetate

The compound of example 427 was prepared analogous to the compound of example 378 by reduction of compound of example 426. Yield: 68%; ¹H NMR (DMSO-d₆, 300 MHz): δ 7.72 (s, 1H), 7.25 (d, 2H), 6.56 (d, 2H), 5.35 (s, 2H), 4.09 (q, 2H), 3.21 (s, 2H), 2.89 (m, 3H), 2.33 (m, 2H), 1.98 (m, 2H), 1.69 (m, 2H), 1.19 (t, 3H); MS: m/z 346 (M+1).

### Example 428

### Ethyl 2-(4-(5-(4-(3-(2-chlorophenyl)ureido)phenyl)thiazol-2-yl)piperidin-1-yl)acetate

The compound of example 428 was prepared analogous to the compound of example 6 by reaction of the compound of example 427 with 2-chloro-1-isocyanatobenzene.

Yield: 87%; ¹H NMR (DMSO-d₆, 300 MHz): δ 9.21 (s, 1H), 8.56 (d, 1H), 8.15 (t, 1H), 7.94 (s, 1H), 7.55 (d, 2H), 7.50 (d, 2H), 7.25 (dd, 1H), 7.14 (t, 1H), 7.02 (m, 1H), 4.09 (q, 2H), 3.21 (s, 2H), 2.95 (m, 3H), 2.35 (m, 2H), 2.01 (m, 2H), 1.75 (m, 2H), 1.19 (t, 3H); MS: m/z 499 (M+1).

### Example 429

### 2-(4-(5-(4-(3-(2-Chlorophenyl)ureido)phenyl)thiazol-2-yl)piperidin-1-yl)acetic acid

The compound of example 429 was prepared analogous to the compound of example 7 by hydrolysis of the compound of example 428. Yield: 69%; ¹H NMR (DMSO-d₆, 300 MHz): δ 9.35 (s, 1H), 8.68 (s, 1H), 8.13 (t, 1H), 7.97 (s, 1H), 7.56 (d, 2H), 7.52 (d, 2H), 7.25 (t, 1H), 7.15 (t, 1H), 7.03 (m, 1H), 3.24 (s, 2H), 3.15 (m, 2H), 3.06 (m, 1H), 2.66 (m, 2H), 2.11 (m, 2H), 1.95 (m, 2H); MS: m/z 471 (M+1).

### Example 430

### Ethyl 2-(4-(5-(4-(3-(2-fluorophenyl)ureido)phenyl)thiazol-2-yl)piperidin-1-yl)acetate

The compound of example 430 was prepared analogous to the compound of example 6 by reaction of the compound of example 427 with 2-fluoro-1-isocyanatobenzene.

Yield: 92%; ¹H NMR (DMSO-d₆, 300 MHz): δ 9.55 (s, 1H), 8.32 (s, 1H), 8.15 (d, 1H), 7.95 (s, 1H), 7.56 (d, 2H), 7.51 (d, 2H), 7.43 (dd, 1H), 7.30 (t, 1H), 7.01 (m, 1H), 4.09 (q, 2H), 3.21 (s, 2H), 2.95 (m, 3H), 2.35 (m, 2H), 2.01 (m, 2H), 1.74 (m, 2H), 1.19 (t, 3H); MS: m/z 483 (M+1).

### Example 431

### 2-(4-(5-(4-(3-(2-Fluorophenyl)ureido)phenyl)thiazol-2-yl)piperidin-1-yl)acetic acid

The compound of example 431 was prepared analogous to the compound of example 7 by hydrolysis of the compound of example 430. Yield: 76%; ¹H NMR (DMSO-d₆, 300 MHz): δ 9.64 (s, 1H), 8.39 (s, 1H), 8.14 (dd, 1H), 7.95 (s, 1H), 7.57 (d, 2H), 7.52 (d, 2H), 7.45 (t, 1H), 7.31 (t, 1H), 7.05 (m, 1H), 3.26 (s, 2H), 3.19 (m, 2H), 3.07 (m, 1H), 2.67 (m, 2H), 2.11 (m, 2H), 1.91 (m, 2H); MS: m/z 455 (M+1).

### Example 432

### Ethyl 2-(4-(5-(4-(3-(2,4-difluorophenyl)ureido)phenyl)thiazol-2-yl)piperidin-1-yl)acetate

The compound of example 432 was prepared analogous to the compound of example 6 by reaction of the compound of example 427 with 2,4-difluoro-1-isocyanatobenzene.

Yield: 85%; ¹H NMR (DMSO-d₆, 300 MHz): δ 9.15 (s, 1H), 8.52 (s, 1H), 8.09 (m, 1H), 7.99 (s, 1H), 7.54 (d, 2H), 7.49 (d, 2H), 7.33 (m, 1H), 7.06 (m, 1H), 4.09 (q, 2H), 3.21 (s, 2H), 2.95 (m, 3H), 2.35 (m, 2H), 2.01 (m, 2H), 1.74 (m, 2H), 1.19 (t, 3H); MS: m/z 501 (M+1).

### Example 433

### 2-(4-(5-(4-(3-(2,4-Difluorophenyl)ureido)phenyl)thiazol-2-yl)piperidin-1-yl)acetic acid

The compound of example 433 was prepared analogous to the compound of example 7 by hydrolysis of the compound of example 432. Yield: 73%; ¹H NMR (DMSO-d₆, 300 MHz): δ 9.35 (s, 1H), 8.65 (s, 1H), 8.05 (m, 1H), 7.97 (s, 1H), 7.56 (d, 2H), 7.51 (d, 2H), 7.33 (t, 1H), 7.03 (t, 1H), 3.43 (s, 2H), 3.27 (m, 2H), 3.11 (m, 1H), 2.79 (m, 2H), 2.14 (m, 2H), 1.96 (m, 2H); MS: m/z 473 (M+1).

### Example 434

### Ethyl 2-(4-(5-(4-(3-(2,4,5-trifluorophenyl)ureido)phenyl)thiazol-2-yl)piperidin-1-yl)acetate

The compound of example 434 was prepared analogous to the compound of example by reaction of the compound of example 427 with 2,4,5-trifluoro-1-isocyanatobenzene. Yield: 87%; ¹H NMR (DMSO-d₆, 300 MHz): δ 9.21 (s, 1H), 8.73 (s, 1H), 8.22 (m, 1H), 7.95 (s, 1H), 7.67 (m, 1H), 7.55 (d, 2H), 7.49 (d, 2H), 4.09 (q, 2H), 3.21 (s, 2H), 2.95 (m, 3H), 2.35 (m, 2H), 2.01 (m, 2H), 1.75 (m, 2H), 1.19 (t, 3H); MS: m/z 519 (M+1).

### Example 435

### 2-(4-(5-(4-(3-(2,4,5-Trifluorophenyl)ureido)phenyl)thiazol-2-yl)piperidin-1-yl)acetic acid

The compound of example 435 was prepared analogous to the compound of example 7 by hydrolysis of the compound of example 434. Yield: 73%; ¹H NMR (DMSO-d₆, 300 MHz): δ 9.39 (s, 1H), 8.87 (s, 1H), 8.18 (m, 1H), 7.98 (s, 1H), 7.64 (m, 1H), 7.57 (d, 2H), 7.51 (d, 2H), 3.40 (s, 2H), 3.23 (m, 2H), 3.09 (m, 1H), 2.73 (m, 2H), 2.13 (m, 2H), 1.93 (m, 2H); MS: m/z 491 (M+1).

### Example 436

### Ethyl 2-(4-(5-(4-(3-(2-(trifluoromethyl)phenyl)ureido)phenyl)thiazol-2-yl)piperidin-1-yl)acetate

The compound of example 436 was prepared analogous to the compound of example by reaction of the compound of example 427 with 1-isocyanato-2-trifluoromethylbenzene. Yield: 80%; ¹H NMR (DMSO-d₆, 300 MHz): δ 9.50 (s, 1H), 8.09 (s, 1H), 7.95 (s, 1H), 7.93 (d, 1H), 7.67 (m, 2H), 7.55 (d, 2H), 7.50 (d, 2H), 7.29 (t, 1H), 4.09 (q, 2H), 3.21 (s, 2H), 2.91 (m, 3H), 2.35 (m, 2H), 2.01 (m, 2H), 1.71 (m, 2H), 1.19 (t, 3H); MS: m/z 533 (M+1).

### Example 437

### 2-(4-(5-(4-(3-(2-(Trifluoromethyl)phenyl)ureido)phenyl)thiazol-2-yl)piperidin-1-yl)acetic acid

The compound of example 437 was prepared analogous to the compound of example 7 by hydrolysis of the compound of example 436. Yield: 79%; ¹H NMR (DMSO-d₆, 300 MHz): δ 9.96 (s, 1H), 8.57 (s, 1H), 7.95 (s, 1H), 7.873 (d, 1H), 7.67 (m, 2H), 7.58 (d, 2H), 7.53 (d, 2H), 7.30 (t, 1H), 3.09 (s, 2H), 3.04 (m, 3H), 2.39 (m, 2H), 2.06 (m, 2H), 1.86 (m, 2H); MS: m/z 505 (M+1).

### Example 438

### Ethyl 2-(4-(5-(4-(3-(2,3,4-trifluorophenyl)ureido)phenyl)thiazol-2-yl)piperidin-1-yl)acetate

The compound of example 438 was prepared analogous to the compound of example by reaction of the compound of example 427 with 2,3,4-trifluoro-1-isocyanatobenzene. Yield: 66%; ¹H NMR (DMSO-d₆, 300 MHz): δ 9.19 (s, 1H), 8.69 (s, 1H), 7.95 (s, 1H), 7.89 (m, 1H), 7.56 (d, 2H), 7.50 (d, 2H), 7.28 (m, 1H), 4.11 (q, 2H), 3.22 (s, 2H), 2.92 (m, 3H), 2.36 (m, 2H), 2.03 (m, 2H), 1.73 (m, 2H), 1.21 (t, 3H); MS: m/z 519 (M+1).

### Example 439

### 2-(4-(5-(4-(3-(2,3,4-Trifluorophenyl)ureido)phenyl)thiazol-2-yl)piperidin-1-yl)acetic acid

The compound of example 439 was prepared analogous to the compound of example 7 by hydrolysis of the compound of example 438. Yield: 87%; ¹H NMR (DMSO-d₆, 300 MHz): δ 9.63 (s, 1H), 8.59 (s, 1H), 7.97 (s, 1H), 7.52 (m, 4H), 7.28 (m, 2H), 3.35 (s, 2H), 3.28 (m, 2H), 3.11 (m, 1H), 2.78 (m, 2H), 2.14 (m, 2H), 1.92 (m, 2H); MS: m/z 491 (M+1).

### Example 440

### Ethyl 2-(4-(5-(4-(3-(2,4,6-trifluorophenyl)ureido)phenyl)thiazol-2-yl)piperidin-1-yl)acetate

The compound of example 440 was prepared analogous to the compound of example by reaction of the compound of example 427 with 2,4,6-trifluoro-1-isocyanatobenzene. Yield: 74%; ¹H NMR (DMSO-d₆, 300 MHz): δ 9.13 (s, 1H), 8.05 (s, 1H), 7.94 (s, 1H), 7.54 (m, 4H), 7.28 (m, 2H), 4.11 (q, 2H), 3.22 (s, 2H), 2.92 (m, 3H), 2.36 (m, 2H), 2.02 (m, 2H), 1.72 (m, 2H), 1.20 (t, 3H); MS: m/z 519 (M+1).

### Example 441

### 2-(4-(5-(4-(3-(2,4,6-Trifluorophenyl)ureido)phenyl)thiazol-2-yl)piperidin-1-yl)acetic acid

The compound of example 441 was prepared analogous to the compound of example 7 by hydrolysis of the compound of example 440. Yield: 92%; ¹H NMR (DMSO-d₆, 300 MHz): δ 9.40 (s, 1H), 8.89 (s, 1H), 7.97 (s, 1H), 7.83 (m, 1H), 7.57 (d, 2H), 7.52 (d, 2H), 7.28 (m, 1H), 3.26 (s, 2H), 3.21 (m, 2H), 3.08 (m, 1H), 2.69 (m, 2H), 2.12 (m, 2H), 1.92 (m, 2H); MS: m/z 491 (M+1).

### Example 442

### Ethyl 2-methyl-2-(4-(5-(4-nitrophenyl)thiazol-2-yl)piperidin-1-yl)propanoate

To a solution of the compound of example 425 (2.50 g, 7.67 mmol) in DMF (35 ml) was added ethyl 2-bromo-2-methylpropanoate (1.706 mL, 11.51 mmol) and potassium carbonate (3.18 g, 23.02 mmol) and the reaction mixture was stirred at 50° C. for 16 h. After completion of the reaction, water was added and the reaction mixture was extracted with ethyl acetate. The ethyl acetate extract was washed with water and brine and dried over sodium sulfate. The solvent was removed to yield a solid, which was purified by column chromatography (silica gel, 30% ethyl acetate in chloroform) to afford the title compound. Yield: 49%; ¹H NMR (DMSO-d₆, 300 MHz): δ 8.35 (s, 1H), 8.27 (d, 2H), 7.92 (d, 2H), 4.11 (q, 2H), 3.00 (m, 3H), 2.28 (m, 2H), 2.02 (m, 2H), 1.69 (m, 2H), 1.25 (s, 6H), 1.22 (t, 3H); MS: m/z 404 (M+1).

### Example 443

### Ethyl 2-(4-(5-(4-aminophenyl)thiazol-2-yl)piperidin-1-yl)-2-methylpropanoate

The compound of example 443 was prepared analogous to the compound of example 378 by reduction of compound of example 442. Yield: 55%; ¹H NMR (DMSO-d₆, 300 MHz): δ 7.74 (s, 1H), 7.26 (d, 2H), 6.58 (d, 2H), 5.37 (s, 2H), 4.12 (q, 2H), 2.98 (m, 2H), 2.90 (m, 1H), 2.27 (m, 2H), 2.02 (m, 2H), 1.67 (m, 2H), 1.24 (s, 6H), 1.22 (t, 3H); MS: m/z 374 (M+1).

### Example 444

### Ethyl 2-methyl-2-(4-(5-(4-(3-(2,4,5-trifluorophenyl)ureido)phenyl)thiazol-2-yl)piperidin-1-yl)propanoate

The compound of example 444 was prepared analogous to the compound of example by reaction of the compound of example 443 with 2,4,6-trifluoro-1-isocyanatobenzene. Yield: 82%; ¹H NMR (DMSO-d₆, 300 MHz): δ 9.24 (s, 1H), 8.75 (s, 1H), 8.21 (m, 1H), 7.97 (s, 1H), 7.67 (m, 1H), 7.57 (d, 2H), 7.51 (d, 2H), 4.12 (q, 2H), 3.00 (m, 2H), 2.93 (m, 1H), 2.29 (m, 2H), 2.05 (m, 2H), 1.70 (m, 2H), 1.25 (s, 6H), 1.22 (t, 3H); MS: m/z 547 (M+1).

### Example 445

### Ethyl 2-(4-(5-(4-(3-(2-fluorophenyl)ureido)phenyl)thiazol-2-yl)piperidin-1-yl)-2-methylpropanoate

The compound of example 445 was prepared analogous to the compound of example 6 by reaction of the compound of example 443 with 2-fluoro-1-isocyanatobenzene.

Yield: 88%; ¹H NMR (DMSO-d₆, 300 MHz): δ 9.23 (s, 1H), 8.58 (s, 1H), 8.16 (m, 1H), 7.96 (s, 1H), 7.56 (d, 2H), 7.52 (d, 2H), 7.26 (dd, 1H), 7.16 (t, 1H), 7.03 (m, 1H), 4.12 (q, 2H), 3.00 (m, 2H), 2.93 (m, 1H), 2.29 (m, 2H), 2.05 (m, 2H), 1.70 (m, 2H), 1.25 (s, 6H), 1.22 (t, 3H); MS: m/z 511 (M+1).

### Example 446

### Ethyl 2-(4-(5-(4-(3-(2-chlorophenyl)ureido)phenyl)thiazol-2-yl)piperidin-1-yl)-2-methylpropanoate

The compound of example 446 was prepared analogous to the compound of example 6 by reaction of the compound of example 443 with 2-chloro-1-isocyanatobenzene.

Yield: 85%; ¹H NMR (DMSO-d₆, 300 MHz): δ 9.57 (s, 1H), 8.35 (s, 1H), 8.17 (d, 1H), 7.97 (s, 1H), 7.57 (d, 2H), 7.53 (d, 2H), 7.47 (d, 1H), 7.31 (t, 1H), 7.04 (m, 1H), 4.13 (q, 2H), 3.00 (m, 2H), 2.92 (m, 1H), 2.29 (m, 2H), 2.05 (m, 2H), 1.68 (m, 2H), 1.25 (s, 6H), 1.22 (t, 3H); MS: m/z 527 (M+1).

### Example 447

### Ethyl 2-(4-(5-(4-(3-(2,4-difluorophenyl)ureido)phenyl)thiazol-2-yl)piperidin-1-yl)-2-methylpropanoate

The compound of example 447 was prepared analogous to the compound of example 6 by reaction of the compound of example 443 with 2,4-difluoro-1-isocyanatobenzene.

Yield: 93%; ¹H NMR (DMSO-d₆, 300 MHz): δ 9.17 (s, 1H), 8.54 (s, 1H), 8.10 (m, 1H), 7.96 (s, 1H), 7.56 (d, 2H), 7.51 (d, 2H), 7.34 (t, 1H), 7.07 (t, 1H), 4.11 (q, 2H), 3.10 (m, 2H), 2.98 (m, 1H), 2.26 (m, 2H), 2.09 (m, 2H), 1.65 (m, 2H), 1.25 (s, 6H), 1.22 (t, 3H); MS: m/z 529 (M+1).

### Example 448

### t-Butyl 2-(4-(5-(4-nitrophenyl)thiazol-2-yl)piperidin-1-yl)propanoate

To a solution of the compound of example 425 (2.50 g, 7.67 mmol) in DMF (35 ml) was added t-butyl 2-bromopropanoate (2.4 g, 11.48 mmol) and potassium carbonate (3.18 g, 23.02 mmol) and the reaction mixture was stirred at 50° C. for 16 h. After completion of the reaction, water was added and the reaction mixture was extracted with ethyl acetate. The ethyl acetate extract was washed with water and brine and dried over sodium sulfate. The solvent was removed to yield a solid, which was purified by column chromatography. Yield: 72%; ¹H NMR (DMSO-d₆, 300 MHz): δ 8.36 (s, 1H), 8.28 (d, 2H), 7.93 (d, 2H), 3.24 (m, 1H), 3.03 (m, 3H), 2.58 (m, 1H), 2.39 (m, 1H), 2.08 (m, 2H), 1.79 (m, 2H), 1.43 (s, 9H), 1.17 (d, 3H); MS: m/z 418 (M+1).

### Example 449

### t-Butyl 2-(4-(5-(4-aminophenyl)thiazol-2-yl)piperidin-1-yl)propanoate

The compound of example 449 was prepared analogous to the compound of example 378 by reduction of compound of example 448. Yield: 86%; ¹H NMR (DMSO-d₆, 300 MHz): δ 7.74 (s, 1H), 7.27 (d, 2H), 6.59 (d, 2H), 5.37 (s, 2H), 3.24 (m, 1H), 2.94 (m, 4H), 2.36 (m, 1H), 2.03 (m, 2H), 1.74 (m, 2H), 1.42 (s, 9H), 1.16 (d, 3H); MS: m/z 388 (M+1).

### Example 450

### t-Butyl 2-(4-(5-(4-(3-(2,4,5-trifluorophenyl)ureido)phenyl)thiazol-2-yl)piperidin-1-yl)propanoate

The compound of example 450 was prepared analogous to the compound of example by reaction of the compound of example 443 with 2,4,5-trifluoro-1-isocyanatobenzene. Yield: 87%; ¹H NMR (DMSO-d₆, 300 MHz): δ 9.23 (s, 1H), 8.75 (s, 1H), 8.24 (s, 1H), 7.97 (s, 1H), 7.69 (m, 1H), 7.57 (d, 2H), 7.52 (d, 2H), 3.25 (m, 1H), 2.99 (m, 3H), 2.56 (m, 1H), 2.37 (m, 1H), 2.01 (m, 2H), 1.75 (m, 2H), 1.43 (s, 9H), 1.16 (d, 3H); MS: m/z 561 (M+1).

### Example 451

### 2-(4-(5-(4-(3-(2,4,5-Trifluorophenyl)ureido)phenyl)thiazol-2-yl)piperidin-1-yl)propanoic acid

The compound of example 451 was prepared analogous to the compound of example 348 by reaction of the compound of example 450 with trifluoroacetic acid. Yield: 87%;

¹H NMR (DMSO-d₆, 300 MHz): δ 9.40 (s, 1H), 8.90 (s, 1H), 8.23 (m, 1H), 7.99 (s, 1H), 7.69 (m, 1H), 7.59 (d, 2H), 7.54 (d, 2H), 3.39 (m, 1H), 3.13 (m, 3H), 2.74 (m, 2H), 2.15 (m, 2H), 1.88 (m, 2H), 1.276 (d, 3H); MS: m/z 505 (M+1).

### Example 452

### t-Butyl 2-(4-(5-(4-(3-(2-fluorophenyl)ureido)phenyl)thiazol-2-yl)piperidin-1-yl)propanoate

The compound of example 452 was prepared analogous to the compound of example 6 by reaction of the compound of example 443 with 2-fluoro-1-isocyanatobenzene.

Yield: 89%; ¹H NMR (DMSO-d₆, 300 MHz): δ 9.23 (s, 1H), 8.59 (s, 1H), 8.15 (m, 1H), 7.96 (s, 1H), 7.57 (d, 2H), 7.52 (d, 2H), 7.25 (m, 1H), 7.15 (m, 1H), 7.03 (m, 1H), 3.23 (m, 1H), 2.96 (m, 3H), 2.53 (m, 1H), 2.38 (m, 1H), 2.01 (m, 2H), 1.75 (m, 2H), 1.43 (s, 9H), 1.16 (d, 3H); MS: m/z 525 (M+1).

### Example 453

### 2-(4-(5-(4-(3-(2-Fluorophenyl)ureido)phenyl)thiazol-2-yl)piperidin-1-yl) propanoic acid

The compound of example 453 was prepared analogous to the compound of example 348 by reaction of the compound of example 452 with trifluoroacetic acid. Yield: 78%;

¹H NMR (DMSO-d₆, 300 MHz): δ 9.37 (s, 1H), 8.66 (s, 1H), 8.16 (t, 1H), 8.02 (s, 1H), 7.59 (d, 2H), 7.55 (d, 2H), 7.27 (d, 1H), 7.17 (m, 1H), 7.05 (m, 1H), 4.11 (m, 1H), 3.39 (m, 3H), 3.25 (m, 2H), 2.27 (m, 2H), 2.12 (m, 2H), 1.49 (d, 3H); MS: m/z 469 (M+1).

### Example 454

### t-Butyl 2-(4-(5-(4-(3-(2-chlorophenyl)ureido)phenyl)thiazol-2-yl)piperidin-1-yl)propanoate

The compound of example 454 was prepared analogous to the compound of example 6 by reaction of the compound of example 443 with 2-chloro-1-isocyanatobenzene.

Yield: 91%; ¹H NMR (DMSO-d₆, 300 MHz): δ 9.57 (s, 1H), 8.34 (s, 1H), 8.18 (m, 1H), 7.97 (s, 1H), 7.58 (d, 2H), 7.54 (d, 2H), 7.48 (m, 1H), 7.34 (m, 1H), 7.07 (m, 1H), 3.23 (m, 1H), 2.96 (m, 3H), 2.57 (m, 1H), 2.38 (m, 1H), 2.01 (m, 2H), 1.72 (m, 2H), 1.43 (s, 9H), 1.17 (d, 3H); MS: m/z 541 (M+1).

### Example 455

### 2-(4-(5-(4-(3-(2-Chlorophenyl)ureido)phenyl)thiazol-2-yl)piperidin-1-yl)propanoic acid

The compound of example 455 was prepared analogous to the compound of example 348 by reaction of the compound of example 454 with trifluoroacetic acid. Yield: 39%;

¹H NMR (DMSO-d₆, 300 MHz): δ 9.65 (s, 1H), 8.39 (s, 1H), 8.16 (m, 1H), 8.03 (s, 1H), 7.60 (d, 2H), 7.56 (d, 2H), 7.48 (m, 1H), 7.33 (m, 1H), 7.07 (m, 1H), 4.18 (m, 1H), 3.43 (m, 3H), 3.35 (m, 2H), 2.28 (m, 2H), 2.13 (m, 2H), 1.51 (d, 3H); MS: m/z 485 (M+1).

### Example 456

### t-Butyl 2-(4-(5-(4-(3-(2,4-difluorophenyl)ureido)phenyl)thiazol-2-yl)piperidin-1-yl)propanoate

The compound of example 456 was prepared analogous to the compound of example 6 by reaction of the compound of example 443 with 2,4-difluoro-1-isocyanatobenzene.

Yield: 93%; ¹H NMR (DMSO-d₆, 300 MHz): δ 9.17 (s, 1H), 8.53 (s, 1H), 8.12 (m, 1H), 7.96 (s, 1H), 7.57 (d, 2H), 7.52 (d, 2H), 7.36 (m, 1H), 7.09 (m, 1H), 3.23 (m, 1H), 2.99 (m, 3H), 2.51 (m, 1H), 2.37 (m, 1H), 2.01 (m, 2H), 1.75 (m, 2H), 1.43 (s, 9H), 1.16 (d, 3H); MS: m/z 543 (M+1).

### Example 457

### 2-(4-(5-(4-(3-(2,4-Difluorophenyl)ureido)phenyl)thiazol-2-yl)piperidin-1-yl)propanoic acid

The compound of example 457 was prepared analogous to the compound of example 348 by reaction of the compound of example 456 with trifluoroacetic acid. Yield: 84%;

¹H NMR (DMSO-d₆, 300 MHz): δ 9.30 (s, 1H), 8.65 (s, 1H), 8.10 (m, 1H), 7.98 (s, 1H), 7.55 (d, 2H), 7.50 (d, 2H), 7.34 (m, 1H), 7.08 (m, 1H), 3.38 (m, 1H), 3.13 (m, 3H), 2.73 (m, 2H), 2.11 (m, 2H), 1.87 (m, 2H), 1.27 (d, 3H); MS: m/z 487 (M+1).

### Example 458

### t-Butyl 2-(4-(5-(4-(3-(2,4,6-trifluorophenyl)ureido)phenyl)thiazol-2-yl)Piperidin-1-yl)propanoate

The compound of example 458 was prepared analogous to the compound of example by reaction of the compound of example 443 with 2,4,6-trifluoro-1-isocyanatobenzene. Yield: 92%; ¹H NMR (DMSO-d₆, 300 MHz): δ 9.15 (s, 1H), 8.07 (s, 1H), 7.95 (s, 1H), 7.55 (d, 2H), 7.51 (d, 2H), 7.31 (m, 2H) 3.22 (m, 1H), 2.95 (m, 3H), 2.56 (m, 1H), 2.37 (m, 1H), 2.01 (m, 2H), 1.75 (m, 2H), 1.43 (s, 9H), 1.16 (d, 3H); MS: m/z 561 (M+1).

### Example 459

### 2-(4-(5-(4-(3-(2,4,6-Trifluorophenyl)ureido)phenyl)thiazol-2-yl)piperidin-1-yl)propanoic acid

The compound of example 459 was prepared analogous to the compound of example 348 by reaction of the compound of example 458 with trifluoroacetic acid. Yield: 94%;

¹H NMR (DMSO-d₆, 300 MHz): δ 9.56 (s, 1H), 8.51 (s, 1H), 7.99 (s, 1H), 7.54 (m, 4H), 7.30 (m, 2H), 3.62 (m, 1H), 3.29 (m, 3H), 2.92 (m, 2H), 2.21 (m, 2H), 1.96 (m, 2H), 1.35 (d, 3H); MS: m/z 505 (M+1).

### Example 460

### t-Butyl 2-methyl-2-(4-(5-(4-nitrophenyl)thiazol-2-yl)piperidin-1-yl)propanoate

To a solution of the compound of example 425 (2.50 g, 7.67 mmol) in DMF (30 mL) was added tert-butyl 2-bromo-2-methylpropanoate (2.410 ml, 12.96 mmol) and potassium carbonate (3.58 g, 25.9 mmol) and the reaction mixture was stirred at 50° C. for 16 h. After completion of the reaction, water was added and the reaction mixture was extracted with ethyl acetate. The ethyl acetate extract was washed with water and brine and dried over sodium sulfate. The solvent was removed to yield a solid, which was purified by column chromatography. Yield: 94%; ¹H NMR (DMSO-d₆, 300 MHz): δ 8.35 (s, 1H), 8.28 (d, 2H), 7.93 (d, 2H), 3.04 (m, 3H), 2.37 (m, 2H), 2.08 (m, 2H), 1.73 (m, 2H), 1.42 (s, 9H), 1.21 (s, 6H); MS: m/z 432 (M+1).

### Example 461

### t-Butyl 2-(4-(5-(4-aminophenyl)thiazol-2-yl)piperidin-1-yl)-2-methyl propanoate

The compound of example 461 was prepared analogous to the compound of example 378 by reduction of compound of example 460. Yield: 62%; ¹H NMR (DMSO-d₆, 300 MHz): δ 7.73 (s, 1H), 7.27 (d, 2H), 6.59 (d, 2H), 5.36 (s, 2H), 3.01 (m, 2H), 2.93 (m, 1H), 2.34 (m, 2H), 2.03 (m, 2H), 1.69 (m, 2H), 1.42 (s, 9H), 1.20 (s, 6H); MS: m/z 402 (M+1).

### Example 462

### t-Butyl 2-methyl-2-(4-(5-(4-(3-(2,4,5-trifluorophenyl)ureido)phenyl)thiazol-2-yl) piperidin-1-yl)propanoate

The compound of example 462 was prepared analogous to the compound of example by reaction of the compound of example 461 with 2,4,5-trifluoro-1-isocyanatobenzene. Yield: 87%; ¹H NMR (DMSO-d₆, 300 MHz): δ 9.23 (s, 1H), 8.75 (s, 1H), 8.24 (m, 1H), 7.97 (s, 1H), 7.69 (m, 1H), 7.57 (d, 2H), 7.52 (d, 2H), 3.02 (m, 2H), 2.36 (m, 1H), 2.06 (m, 2H), 1.71 (m, 2H), 1.69 (m, 2H), 1.42 (s, 9H), 1.21 (s, 6H); MS: m/z 575 (M+1).

### Example 463

### 2-Methyl-2-(4-(5-(4-(3-(2,4,5-trifluorophenyl)ureido)phenyl)thiazol-2-yl)piperidin-1-yl)propanoic acid

To a solution of the compound of example 462 (30 mg, 0.052 mmol) in MeOH (3 mL) was added HCl in isopropanol (0.016 mL, 0.522 mmol) and the reaction mixture was stirred for 16 h. After completion of the reaction, solvent was removed and the solid obtained was triturated with diethyl ether. The solid obtained was filtered and dried to afford title compound. Yield: 78%; ¹H NMR (DMSO-d₆, 300 MHz): δ 9.89 (s, 1H), 9.84 (s, 1H), 9.04 (s, 1H), 8.21 (m, 1H), 8.03 (s, 1H), 7.66 (m, 1H), 7.60 (d, 2H), 7.55 (d, 2H), 3.53 (m, 2H), 3.40 (m, 1H), 3.28 (m, 2H), 2.33 (m, 4H), 1.57 (s, 6H); MS: m/z 519 (M+1).

### Example 464

### t-Butyl 2-(4-(5-(4-(3-(2-fluorophenyl)ureido)phenyl)thiazol-2-yl)piperidin-1-yl)-2-methylpropanoate

The compound of example 464 was prepared analogous to the compound of example 6 by reaction of the compound of example 461 with 2-fluoro-1-isocyanatobenzene.

Yield: 86%; ¹H NMR (DMSO-d₆, 300 MHz): δ 9.22 (s, 1H), 8.58 (s, 1H), 8.18 (m, 1H), 7.96 (s, 1H), 7.57 (d, 2H), 7.52 (d, 2H), 7.28 (m, 1H), 7.15 (m, 1H), 7.03 (m, 1H), 3.03 (m, 3H), 2.36 (m, 2H), 2.06 (m, 2H), 1.68 (m, 2H), 1.42 (s, 9H), 1.21 (s, 6H); MS: m/z 539 (M+1).

### Example 465

### 2-(4-(5-(4-(3-(2-Fluorophenyl)ureido)phenyl)thiazol-2-yl)piperidin-1-yl)-2-methylpropanoic acid

The compound of example 465 was prepared analogous to the compound of example 463 by reaction of the compound of example 464 with HCl in isopropanol. Yield: 80%;

¹H NMR (DMSO-d₆, 300 MHz): δ 9.93 (s, 1H), 9.74 (s, 1H), 8.81 (s, 1H), 8.15 (m, 1H), 8.03 (s, 1H), 7.60 (d, 2H), 7.56 (d, 2H), 7.27 (m, 1H), 7.14 (m, 1H), 7.04 (m, 1H), 3.54 (m, 2H), 3.40 (m, 1H), 3.28 (m, 2H), 2.28 (m, 4H), 1.57 (s, 6H); MS: m/z 483 (M+1).

### Example 466

### t-Butyl 2-(4-(5-(4-(3-(2-chlorophenyl)ureido)phenyl)thiazol-2-yl)piperidin-1-yl)-2-methylpropanoate

The compound of example 466 was prepared analogous to the compound of example 6 by reaction of the compound of example 461 with 2-chloro-1-isocyanatobenzene.

Yield: 80%; ¹H NMR (DMSO-d₆, 300 MHz): δ 9.57 (s, 1H), 8.34 (s, 1H), 8.18 (m, 1H), 7.96 (s, 1H), 7.58 (d, 2H), 7.53 (d, 2H), 7.48 (m, 1H), 7.33 (m, 1H), 7.06 (m, 1H), 3.03 (m, 3H), 2.36 (m, 2H), 2.06 (m, 2H), 1.68 (m, 2H), 1.42 (s, 9H), 1.21 (s, 6H); MS: m/z 555 (M+1).

### Example 467

### 2-(4-(5-(4-(3-(2-Chlorophenyl)ureido)phenyl)thiazol-2-yl)piperidin-1-yl)-2-methyl propanoic acid

The compound of example 467 was prepared analogous to the compound of example 463 by reaction of the compound of example 466 with HCl in isopropanol. Yield: 79%;

¹H NMR (DMSO-d₆, 300 MHz): δ 10.05 (s, 1H), 9.70 (s, 1H), 8.55 (s, 1H), 8.15 (d, 1H), 8.03 (s, 1H), 7.58 (m, 4H), 7.47 (d, 1H), 7.32 (m, 1H), 7.06 (m, 1H), 3.54 (m, 2H), 3.40 (m, 1H), 3.28 (m, 2H), 2.28 (m, 4H), 1.57 (s, 6H); MS: m/z 500 (M+1).

### Example 468

### t-Butyl 2-(4-(5-(4-(3-(2,4-difluorophenyl)ureido)phenyl)thiazol-2-yl)piperidin-1-yl)-2-methylpropanoate

The compound of example 468 was prepared analogous to the compound of example 6 by reaction of the compound of example 461 with 2,4-difluoro-1-isocyanatobenzene.

Yield: 90%; ¹H NMR (DMSO-d₆, 300 MHz): δ 9.17 (s, 1H), 8.54 (s, 1H), 8.09 (m, 1H), 7.96 (s, 1H), 7.56 (d, 2H), 7.52 (d, 2H), 7.32 (m, 1H), 7.06 (m, 1H), 3.03 (m, 3H), 2.36 (m, 2H), 2.06 (m, 2H), 1.68 (m, 2H), 1.42 (s, 9H), 1.21 (s, 6H); MS: m/z 557 (M+1).

### Example 469

### 2-(4-(5-(4-(3-(2,4-Difluorophenyl)ureido)phenyl)thiazol-2-yl)piperidin-1-yl)-2-methylpropanoic acid

The compound of example 469 was prepared analogous to the compound of example 463 by reaction of the compound of example 468 with HCl in isopropanol. Yield: 79%;

¹H NMR (DMSO-d₆, 300 MHz): δ 9.86 (s, 1H), 9.64 (s, 1H), 8.76 (s, 1H), 8.10 (m, 1H), 8.02 (s, 1H), 7.59 (d, 2H), 7.55 (d, 2H), 7.34 (m, 1H), 7.08 (m, 1H), 3.50 (m, 2H), 3.40 (m, 1H), 3.27 (m, 2H), 2.27 (m, 4H), 1.57 (s, 6H); MS: m/z 501 (M+1).

### Example 470

### t-Butyl 2-methyl-2-(4-(5-(4-(3-(2,4,6-trifluorophenyl)ureido)phenyl)thiazol-2-yl)Piperidin-1-yl)propanoate

The compound of example 470 was prepared analogous to the compound of example 6 by reaction of the compound of example 461 with 2,4,6-trifluoro-1-isocyanatobenzene. Yield: 87%; ¹H NMR (DMSO-d₆, 300 MHz): δ 9.15 (s, 1H), 8.07 (s, 1H), 7.96 (s, 1H), 7.55 (d, 2H), 7.51 (d, 2H), 7.30 (m, 2H), 3.03 (m, 3H), 2.36 (m, 2H), 2.05 (m, 2H), 1.67 (m, 2H), 1.42 (s, 9H), 1.21 (s, 6H); MS: m/z 575 (M+1).

### Example 471

### 2-Methyl-2-(4-(5-(4-(3-(2,4,6-trifluorophenyl)ureido)phenyl)thiazol-2-yl)piperidin-1-yl)propanoic acid

The compound of example 471 was prepared analogous to the compound of example 463 by reaction of the compound of example 470 with HCl in isopropanol.

Yield: 87%; ¹H NMR (DMSO-d₆, 300 MHz): δ 9.93 (s, 1H), 9.67 (s, 1H), 8.45 (s, 1H), 8.02 (m, 1H), 7.58 (d, 2H), 7.53 (d, 2H), 7.30 (m, 1H), 3.53 (m, 2H), 3.41 (m, 1H), 3.28 (m, 2H), 2.27 (m, 4H), 1.57 (s, 6H); MS: m/z 519 (M+1).

### Example 472

### t-Butyl 4-(5-(4-aminophenyl)thiazol-2-yl)piperidine-1-carboxylate

The compound of example 472 was prepared analogous to the compound of example 378 by reduction of compound of example 424. Yield: 87%; ¹H NMR (DMSO-d₆, 300 MHz): δ 7.75 (s, 1H), 7.27 (d, 2H), 6.59 (d, 2H), 5.38 (s, 2H), 4.01 (m, 2H), 3.17 (m, 1H), 2.88 (m, 2H), 2.02 (m, 2H), 1.60 (m, 2H), 1.04 (s, 9H); MS: m/z 360 (M+1).

### Example 473

### t-Butyl 4-(5-(4-(3-(2-chlorophenyl)ureido)phenyl)thiazol-2-yl)piperidine-1-carboxylate

The compound of example 473 was prepared analogous to the compound of example 6 by reaction of the compound of example 472 with 2-chloro-1-isocyanatobenzene.

Yield: 88%; ¹H NMR (DMSO-d₆, 300 MHz): δ 9.58 (s, 1H), 8.35 (s, 1H), 8.18 (dd, 1H), 7.99 (s, 1H), 7.59 (d, 2H), 7.54 (d, 2H), 7.48 (s, 1H), 7.33 (m, 1H), 7.07 (m, 1H), 4.02 (m, 2H), 3.23 (m, 1H), 2.91 (m, 2H), 2.09 (m, 2H), 1.62 (m, 2H), 1.04 (s, 9H); MS: m/z 513 (M+1).

### Example 474

### 1-(2-Chlorophenyl)-3-(4-(2-(piperidin-4-yl)thiazol-5-yl)phenyl)urea hydrochloride

To a solution of the compound of example 473 (50 mg, 0.097 mmol) was added HCl in dioxane (1 mL, 0.097 mmol) and the reaction mixture was stirred at room temperature for 3-4 h. After completion of the reaction, solvent was removed and the material obtained was triturated with diethyl ether to obtain a solid, which was filtered and dried to afford the title compound. Yield: 80%; ¹H NMR (DMSO-d₆, 300 MHz): δ 9.03 (s, 1H), 8.84 (s, 1H), 8.51 (s, 1H), 8.16 (d, 1H), 8.02 (s, 1H), 7.60 (d, 2H), 7.53 (d, 2H), 7.47 (s, 1H), 7.33 (m, 1H), 7.06 (m, 1H), 3.39 (m, 3H), 3.09 (m, 2H), 2.22 (m, 2H), 2.00 (m, 2H); MS: m/z 413 (M+1).

### Example 475

### t-Butyl 4-(5-(4-(3-(2-fluorophenyl)ureido)phenyl)thiazol-2-yl)piperidine-1-carboxylate

The compound of example 475 was prepared analogous to the compound of example 6 by reaction of the compound of example 472 with 2-fluoro-1-isocyanatobenzene.

Yield: 80%; ¹H NMR (DMSO-d₆, 300 MHz): δ 9.23 (s, 1H), 8.59 (s, 1H), 8.17 (m, 1H), 7.98 (s, 1H), 7.58 (d, 2H), 7.53 (d, 2H), 7.28 (s, 1H), 7.17 (m, 1H), 7.05 (m, 1H), 4.03 (m, 2H), 3.23 (m, 1H), 2.91 (m, 2H), 2.09 (m, 2H), 1.62 (m, 2H), 1.41 (s, 9H); MS: m/z 497 (M+1).

### Example 476

### 1-(2-Fluorophenyl)-3-(4-(2-(piperidin-4-yl)thiazol-5-yl)phenyl)urea hydrochloride

The compound of example 476 was prepared analogous to the compound of example 474 by reaction of the compound of example 475 with HCl in dioxane. Yield: 74%; ¹H NMR (DMSO-d₆, 300 MHz): δ 9.66 (s, 1H), 8.94 (s, 1H), 8.77 (s, 1H), 8.15 (s, 1H), 8.02 (s, 1H), 7.59 (d, 2H), 7.55 (d, 2H), 7.27 (m, 1H), 7.17 (m, 1H), 7.04 (m, 1H), 3.39 (m, 3H), 3.07 (m, 2H), 2.22 (m, 2H), 2.00 (m, 2H); MS: m/z 397 (M+1).

### Example 477

### t-Butyl 4-(5-(4-(3-(2,4-difluorophenyl)ureido)phenyl)thiazol-2-yl)piperidine-1-carboxylate

The compound of example 477 was prepared analogous to the compound of example 6 by reaction of the compound of example 472 with 2,4-difluoro-1-isocyanatobenzene.

Yield: 87%; ¹H NMR (DMSO-d₆, 300 MHz): δ 9.18 (s, 1H), 8.54 (s, 1H), 8.12 (m, 1H), 7.98 (s, 1H), 7.57 (d, 2H), 7.52 (d, 2H), 7.36 (s, 1H), 7.09 (m, 1H), 4.03 (m, 2H), 3.18 (m, 1H), 2.91 (m, 2H), 2.09 (m, 2H), 1.62 (m, 2H), 1.41 (s, 9H); MS: m/z 515 (M+1).

### Example 478

### 1-(2,4-Difluorophenyl)-3-(4-(2-(piperidin-4-yl)thiazol-5-yl)phenyl)urea hydrochloride

The compound of example 478 was prepared analogous to the compound of example 474 by reaction of the compound of example 477 with HCl in dioxane. Yield: 87%; ¹H NMR (DMSO-d₆, 300 MHz): δ 9.61 (s, 1H), 8.95 (s, 1H), 8.77 (s, 1H), 8.08 (s, 1H), 8.01 (s, 1H), 7.58 (d, 2H), 7.54 (d, 2H), 7.34 (m, 1H), 7.05 (m, 1H), 3.39 (m, 3H), 3.07 (m, 2H), 2.22 (m, 2H), 1.96 (m, 2H); MS: m/z 415 (M+1).

### Example 479

### t-Butyl 4-(5-(4-(3-(2,4,6-trifluorophenyl)ureido)phenyl)thiazol-2-yl)piperidine-1-carboxylate

The compound of example 479 was prepared analogous to the compound of example by reaction of the compound of example 472 with 2,4,5-trifluoro-1-isocyanatobenzene. Yield: 84%; ¹H NMR (DMSO-d₆, 300 MHz): δ 9.16 (s, 1H), 8.08 (s, 1H), 7.97 (s, 1H), 7.56 (d, 2H), 7.51 (d, 2H), 7.31 (m, 2H), 4.02 (m, 2H), 3.20 (m, 1H), 2.91 (m, 2H), 2.05 (m, 2H), 1.58 (m, 2H), 1.41 (s, 9H); MS: m/z 533 (M+1).

### Example 480

### 1-(4-(2-(Piperidin-4-yl)thiazol-5-yl)phenyl)-3-(2,4,5-trifluorophenyl)urea hydrochloride

The compound of example 480 was prepared analogous to the compound of example 474 by reaction of the compound of example 479 with HCl in dioxane. Yield: 89%; ¹H NMR (DMSO-d₆, 300 MHz): δ 9.54 (s, 1H), 8.97 (s, 1H), 8.35 (s, 1H), 8.01 (s, 1H), 7.57 (d, 2H), 7.52 (d, 2H), 7.30 (m, 2H), 3.38 (m, 3H), 3.09 (m, 2H), 2.22 (m, 2H), 1.99 (m, 2H); MS: m/z 433 (M+1).

### Example 481

### 5-(4-Nitrophenyl)-2-(1-((trifluoromethyl)sulfonyl)piperidin-4-yl)thiazole

To a solution of the compound of example 425 (1 g, 3.07 mmol) in dichloromethane (15 mL) was added triethylamine (1.283 mL, 9.21 mmol) and stirred for 5 min at room temperature. To the reaction mixture, triflic anhydride (0.622 mL, 3.68 mmol) was added slowly and stirred at room temperature for 16 h. After completion of the reaction, the solvent was removed and the material obtained was purified by column chromatography (silica gel, 30% ethyl acetate in petroleum ether) to afford the title compound. Yield: 62%; ¹H NMR (DMSO-d₆, 300 MHz): δ 8.40 (s, 1H), 8.29 (d, 2H), 7.95 (d, 2H), 3.93 (m, 2H), 3.45 (m, 3H), 2.27 (m, 2H), 1.79 (m, 2H); MS: m/z 422 (M+1).

### Example 482

### 4-(2-(1-((Trifluoromethyl)sulfonyl)piperidin-4-yl)thiazol-5-yl)aniline

The compound of example 482 was prepared analogous to the compound of example 378 by reduction of compound of example 481. Yield: 81%; ¹H NMR (DMSO-d₆, 300 MHz): δ 7.79 (s, 1H), 7.28 (d, 2H), 6.59 (d, 2H), 5.40 (s, 2H), 3.90 (m, 2H), 3.42 (m, 3H), 2.20 (m, 2H), 1.74 (m, 2H); MS: m/z 392 (M+1).

### Example 483

### 1-(2-Fluorophenyl)-3-(4-(2-(1-((trifluoromethyl)sulfonyl)piperidin-4-yl)thiazol-5-yl)phenyl)urea

The compound of example 483 was prepared analogous to the compound of example 6 by reaction of the compound of example 482 with 2-fluoro-1-isocyanatobenzene.

Yield: 90%; ¹H NMR (DMSO-d₆, 300 MHz): δ 9.23 (s, 1H), 8.58 (s, 1H), 8.16 (t, 1H), 8.01 (s, 1H), 7.58 (d, 2H), 7.53 (d, 2H), 7.27 (m, 1H), 7.20 (m, 1H), 7.02 (m, 1H), 3.91 (m, 2H), 3.43 (m, 3H), 2.23 (m, 2H), 1.76 (m, 2H); MS: m/z 529 (M+1).

### Example 484

### 1-(2-Chlorophenyl)-3-(4-(2-(1-((trifluoromethyl)sulfonyl)piperidin-4-yl)thiazol-5-yl)phenyl)urea

The compound of example 484 was prepared analogous to the compound of example 6 by reaction of the compound of example 482 with 2-chloro-1-isocyanatobenzene. Yield: 93%; ¹H NMR (DMSO-d₆, 300 MHz): δ 9.59 (s, 1H), 8.35 (s, 1H), 8.17 (d, 1H), 8.01 (s, 1H), 7.59 (d, 2H), 7.54 (d, 2H), 7.47 (d, 1H), 7.33 (t, 1H), 7.06 (t, 1H), 3.91 (m, 2H), 3.43 (m, 3H), 2.23 (m, 2H), 1.76 (m, 2H); MS: m/z 546 (M+1).

### Example 485

### 1-(2,4-Difluorophenyl)-3-(4-(2-(1-((trifluoromethyl)sulfonyl)piperidin-4-yl)thiazol-5-yl)phenyl)urea

The compound of example 485 was prepared analogous to the compound of example 6 by reaction of the compound of example 482 with 2,4-difluoro-1-isocyanatobenzene.

Yield: 92%; ¹H NMR (DMSO-d₆, 300 MHz): δ 9.18 (s, 1H), 8.53 (s, 1H), 8.11 (m, 1H), 8.00 (s, 1H), 7.58 (d, 2H), 7.52 (d, 2H), 7.35 (m, 1H), 7.05 (m, 1H), 3.91 (m, 2H), 3.43 (m, 3H), 2.23 (m, 2H), 1.76 (m, 2H); MS: m/z 547 (M+1).

### Example 486

### 1-(4-(2-(1-((Trifluoromethyl)sulfonyl)piperidin-4-yl)thiazol-5-yl)phenyl)-3-(2,4,6-trifluorophenyl)urea

The compound of example 486 was prepared analogous to the compound of example by reaction of the compound of example 482 with 2,4,6-trifluoro-1-isocyanatobenzene. Yield: 87%; ¹H NMR (DMSO-d₆, 300 MHz): δ 9.17 (s, 1H), 8.08 (s, 1H), 8.00 (s, 1H), 7.56 (d, 2H), 7.51 (d, 2H), 7.30 (m, 2H), 3.91 (m, 2H), 3.43 (m, 3H), 2.23 (m, 2H), 1.76 (m, 2H); MS: m/z 565 (M+1).

### Example 487

### 1-(4-(2-(1-((Trifluoromethyl)sulfonyl)piperidin-4-yl)thiazol-5-yl)phenyl)-3-(2,4,5-trifluorophenyl)urea

The compound of example 487 was prepared analogous to the compound of example 6 by reaction of the compound of example 482 with 2,4,5-trifluoro-1-isocyanatobenzene. Yield: 87%; ¹H NMR (DMSO-d₆, 300 MHz): δ 9.24 (s, 1H), 8.75 (s, 1H), 8.24 (m, 1H), 8.01 (s, 1H), 7.68 (m, 1H), 7.59 (d, 2H), 7.52 (d, 2H), 3.91 (m, 2H), 3.43 (m, 3H), 2.23 (m, 2H), 1.80 (m, 2H); MS: m/z 565 (M+1).

### Example 488

### 2-(1-(Methylsulfonyl)piperidin-4-yl)-5-(4-nitrophenyl)thiazole

To a solution of the compound of example 425 (1 g, 3.07 mmol) in DCM (15 mL) was added triethylamine (0.279 mL, 2 mmol) and the reaction mixture was stirred for 5 min at room temperature. To the reaction mixture, methanesulfonyl chloride (0.287 mL, 3.68 mmol) was added slowly and stirred at room temperature for 16 h. After completion of the reaction, the solvent was removed and the material obtained was purified by column chromatography (silica gel, 30% ethyl acetate in chloroform) to afford the title compound. Yield: 83%; ¹H NMR (DMSO-d₆, 300 MHz): δ 8.39 (s, 1H), 8.29 (d, 2H), 7.94 (d, 2H), 3.67 (m, 2H), 3.27 (m, 1H), 2.95 (m, 2H), 2.90 (s, 3H), 2.21 (m, 2H), 1.85 (m, 2H); MS: m/z 368 (M+1).

### Example 489

### 4-(2-(1-(Methylsulfonyl)piperidin-4-yl)thiazol-5-yl)aniline

The compound of example 489 was prepared analogous to the compound of example 378 by reduction of compound of example 488. Yield: 82%; ¹H NMR (DMSO-d₆, 300 MHz): δ 7.78 (s, 1H), 7.28 (d, 2H), 6.60 (d, 2H), 5.39 (s, 2H), 3.64 (m, 2H), 3.10 (m, 1H), 2.93 (m, 2H), 2.89 (s, 3H), 2.16 (m, 2H), 1.75 (m, 2H); MS: m/z 338 (M+1).

### Example 490

### 1-(2-Chlorophenyl)-3-(4-(2-(1-(methylsulfonyl)piperidin-4-yl)thiazol-5-yl)phenyl)urea

The compound of example 490 was prepared analogous to the compound of example 6 by reaction of the compound of example 489 with 2-chloro-1-isocyanatobenzene.

Yield: 78%; ¹H NMR (DMSO-d₆, 300 MHz): δ 9.58 (s, 1H), 8.35 (s, 1H), 8.18 (d, 1H), 8.00 (s, 1H), 7.59 (d, 2H), 7.54 (d, 2H), 7.48 (d, 1H), 7.31 (m, 1H), 7.07 (m, 1H), 3.65 (m, 2H), 3.20 (m, 1H), 2.95 (m, 2H), 2.90 (s, 3H), 2.20 (m, 2H), 1.83 (m, 2H); MS: m/z 492 (M+1).

### Example 491

### 1-(2-Fluorophenyl)-3-(4-(2-(1-(methylsulfonyl)piperidin-4-yl)thiazol-5-yl) phenyl)urea

The compound of example 491 was prepared analogous to the compound of example 6 by reaction of the compound of example 489 with 2-fluoro-1-isocyanatobenzene.

Yield: 85%; ¹H NMR (DMSO-d₆, 300 MHz): δ 9.24 (s, 1H), 8.95 (s, 1H), 8.18 (m, 1H), 8.00 (s, 1H), 7.58 (d, 2H), 7.53 (d, 2H), 7.27 (d, 1H), 7.17 (m, 1H), 7.05 (m, 1H), 3.65 (m, 2H), 3.19 (m, 1H), 2.94 (m, 2H), 2.90 (s, 3H), 2.19 (m, 2H), 1.83 (m, 2H); MS: m/z 475 (M+1).

### Example 492

### 1-(2,4-Difluorophenyl)-3-(4-(2-(1-(methylsulfonyl)piperidin-4-yl)thiazol-5-yl) phenyl)urea

The compound of example 492 was prepared analogous to the compound of example 6 by reaction of the compound of example 489 with 2,4-difluoro-1-isocyanatobenzene.

Yield: 75%; ¹H NMR (DMSO-d₆, 300 MHz): δ 9.19 (s, 1H), 8.54 (s, 1H), 8.12 (m, 1H), 8.00 (s, 1H), 7.58 (d, 2H), 7.52 (d, 2H), 7.36 (m, 1H), 7.08 (m, 1H), 3.65 (m, 2H), 3.19 (m, 1H), 2.94 (m, 2H), 2.90 (s, 3H), 2.19 (m, 2H), 1.81 (m, 2H); MS: m/z 493 (M+1).

### Example 493

### 1-(4-(2-(1-(Methylsulfonyl)piperidin-4-yl)thiazol-5-yl)phenyl)-3-(2,4,6-trifluoro phenyl)urea

The compound of example 493 was prepared analogous to the compound of example by reaction of the compound of example 489 with 2,4,6-trifluoro-1-isocyanatobenzene. Yield: 78%; ¹H NMR (DMSO-d₆, 300 MHz): δ 9.25 (s, 1H), 8.76 (s, 1H), 8.24 (m, 1H), 8.01 (s, 1H), 7.69 (m, 1H), 7.59 (d, 2H), 7.52 (d, 2H), 3.65 (m, 2H), 3.19 (m, 1H), 2.94 (m, 2H), 2.90 (s, 3H), 2.19 (m, 2H), 1.81 (m, 2H); MS: m/z 511 (M+1).

### Example 494

### 1-(4-(2-(1-(Methylsulfonyl)piperidin-4-yl)thiazol-5-yl)phenyl)-3-(2,4,5-trifluoro phenyl)urea

The compound of example 494 was prepared analogous to the compound of example by reaction of the compound of example 489 with 2,4,5-trifluoro-1-isocyanatobenzene. Yield: 98%; ¹H NMR (DMSO-d₆, 300 MHz): δ 9.24 (s, 1H), 8.75 (s, 1H), 8.22 (m, 1H), 8.00 (s, 1H), 7.66 (m, 1H), 7.59 (d, 2H), 7.52 (d, 2H), 3.65 (m, 2H), 3.16 (m, 1H), 2.94 (m, 2H), 2.90 (s, 3H), 2.19 (m, 2H), 1.82 (m, 2H); MS: m/z 511 (M+1).

### Example 495

### 3-(Methoxycarbonyl)adamantane-1-carboxylic acid

Commercially available dimethyl adamantane-1,3-dicarboxylate (25 g, 99 mmol) and potassium hydroxide (5.56 g, 99 mmol) were taken in methanol (300 mL) and stirred at 65° C. for 16 h. After completion of the reaction, the solvent was removed and the material obtained was poured into water and this solution was extracted with diethyl ether to remove starting material. The aqueous layer was acidified with dilute HCl and extracted with dichloromethane. The organic layer was washed with water and brine, dried over sodium sulfate and concentrated to afford the title compound. Yield: 90%;

¹H NMR (DMSO-d₆, 300 MHz): δ 12.15 (s, 1H), 3.56 (s, 3H), 2.04 (m, 2H), 1.84 (m, 2H), 1.81 (m, 8H), 1.59 (m, 2H); MS: m/z 239 (M+1).

### Example 496

### Methyl 3-((2-(4-nitrophenyl)-2-oxoethyl)carbamoyl)adamantane-1-carboxylate

To the compound of example 495 (5.00 g, 20.98 mmol) in DMF (40 mL) was added HATU (8.78 g, 23.08 mmol) and the reaction mixture was stirred for 15 min at room temperature. The compound of example 2 (5.45 g, 25.2 mmol) was added to it at room temperature and after 10 min of stirring, DIPEA (8.14 g, 63.0 mmol) was added slowly. After completion of the reaction, it was cooled to room temperature, water (85 mL) was added and the reaction mixture was extracted with ethyl acetate (30 mL×3). The organic layer was passed through Celite® to removed insoluble solid and the organic layer was washed with 3N HCl, aqueous NaHCO₃, concentrated to yield a solid, which was purified by column chromatography (silica gel, 30% ethyl acetate in chloroform) to afford the title compound. Yield: 64%; ¹H NMR (DMSO-d₆, 300 MHz): δ 8.37 (d, 2H), 8.16 (d, 2H), 7.99 (t, 1H), 4.52 (d, 2H), 3.57 (s, 3H), 2.06 (m, 2H), 1.94 (s, 2H), 1.79 (m, 8H), 1.59 (m, 2H); MS: m/z 401 (M+1).

### Example 497

### Methyl 3-(5-(4-nitrophenyl)thiazol-2-yl)adamantane-1-carboxylate

To a solution of the compound of example 496 (1.8 g, 4.83 mmol) in dioxane (20 mL) was added Lawesson's reagent (2.150 g, 5.32 mmol) and the reaction mixture was stirred at 55° C. for 3 h. After completion of the reaction, the reaction mixture was cooled to room temperature, basified with aqueous NaHCO₃ and extracted with ethyl acetate. The organic layer was washed with water and brine solution, dried over sodium sulfate, and concentrated to yield a solid, which was purified by column chromatography (silica gel, 30% ethyl acetate in chloroform) to afford the title compound. Yield: 75%; ¹H NMR (DMSO-d₆, 300 MHz): δ 8.35 (s, 1H), 8.26 (d, 2H), 7.92 (d, 2H), 3.59 (s, 3H), 2.17 (m, 2H), 2.09 (m, 2H), 1.96 (m, 4H), 1.84 (m, 4H), 1.69 (m, 2H); MS: m/z 399 (M+1).

### Example 498

### Methyl 3-(5-(4-aminophenyl)thiazol-2-yl)adamantane-1-carboxylate

The compound of example 498 was prepared analogous to the compound of example 378 by reduction of compound of example 497. Yield: 75%; ¹H NMR (DMSO-d₆, 300 MHz): δ 7.72 (s, 1H), 7.25 (d, 2H), 6.56 (d, 2H), 5.35 (s, 2H), 3.58 (s, 3H), 2.14 (m, 2H), 2.04 (m, 2H), 1.96 (m, 4H), 1.87 (m, 4H), 1.67 (m, 2H); MS: m/z 369 (M+1).

### Example 499

### Methyl 3-(5-(4-(3-(2-chlorophenyl)ureido)phenyl)thiazol-2-yl)adamantane-1-carboxylate

The compound of example 462 was prepared analogous to the compound of example 6 by reaction of the compound of example 498 with 2-chloro-1-isocyanatobenzene.

Yield: 89%; ¹H NMR (DMSO-d₆, 300 MHz): δ 9.54 (s, 1H), 8.32 (s, 1H), 8.15 (dd, 1H), 7.95 (s, 1H), 7.56 (m, 4H), 7.45 (dd, 1H), 7.31 (t, 1H), 7.04 (t, 1H), 3.59 (s, 3H), 2.16 (s, 2H), 2.07 (s, 2H), 1.94 (s, 4H), 1.88 (s, 4H), 1.69 (s, 1H), 1.20 (s, 1H); MS: m/z 523 (M+1).

### Example 500

### 3-(5-(4-(3-(2-Chlorophenyl)ureido)phenyl)thiazol-2-yl)adamantane-1-carboxylic acid

The compound of example 500 was prepared analogous to the compound of example 7 by hydrolysis of the compound of example 499. Yield: 87%; ¹H NMR (DMSO-d₆, 300 MHz): δ 12.21 (s, 1H), 9.66 (s, 1H), 8.42 (s, 1H), 8.14 (dd, 1H), 7.95 (s, 1H), 7.56 (m, 4H), 7.45 (dd, 1H), 7.30 (t, 1H), 7.04 (t, 1H), 2.49 (s, 2H), 2.04 (s, 2H), 1.97 (s, 4H), 1.85 (s, 4H), 1.68 (s, 1H), 1.20 (s, 1H); MS: m/z 508 (M+1).

### Example 501

### Methyl 3-(5-(4-(3-(2-fluorophenyl)ureido)phenyl)thiazol-2-yl)adamantane-1-carboxylate

The compound of example 501 was prepared analogous to the compound of example 6 by reaction of the compound of example 498 with 2-fluoro-1-isocyanatobenzene.

Yield: 89%; ¹H NMR (DMSO-d₆, 300 MHz): δ 9.20 (s, 1H), 8.57 (s, 1H), 8.15 (t, 1H), 7.95 (s, 1H), 7.55 (m, 4H), 7.25 (dd, 1H), 7.15 (t, 1H), 7.02 (m, 1H), 3.59 (s, 3H), 2.16 (s, 2H), 2.07 (s, 2H), 1.94 (s, 4H), 1.83 (s, 4H), 1.69 (s, 2H); MS: m/z 506 (M+1).

### Example 502

### 3-(5-(4-(3-(2-Fluorophenyl)ureido)phenyl)thiazol-2-yl)adamantane-1-carboxylic acid

The compound of example 502 was prepared analogous to the compound of example 7 by hydrolysis of the compound of example 501. Yield: 82%; ¹H NMR (DMSO-d₆, 300 MHz): δ 12.19 (s, 1H), 9.37 (s, 1H), 8.71 (s, 1H), 8.13 (t, 1H), 7.94 (s, 1H), 7.55 (m, 4H), 7.24 (t, 1H), 7.14 (t, 1H), 7.02 (t, 1H), 2.14 (s, 2H), 2.04 (s, 2H), 1.93 (s, 4H), 1.81 (s, 4H), 1.68 (s, 1H), 1.20 (s, 1H); MS: m/z 492 (M+1).

### Example 503

### Methyl 3-(5-(4-(3-(2,4-difluorophenyl)ureido)phenyl)thiazol-2-yl)adamantine-1-carboxylate

The compound of example 503 was prepared analogous to the compound of example 6 by reaction of the compound of example 498 with 2,4-difluoro-1-isocyanatobenzene.

Yield: 94%; ¹H NMR (DMSO-d₆, 300 MHz): δ 9.15 (s, 1H), 8.52 (s, 1H), 8.06 (t, 1H), 7.94 (s, 1H), 7.55 (m, 4H), 7.29 (m, 1H), 7.03 (m, 1H), 3.59 (s, 3H), 2.16 (s, 2H), 2.07 (s, 2H), 1.94 (s, 4H), 1.83 (s, 4H), 1.69 (s, 2H); MS: m/z 524 (M+1).

### Example 504

### 3-(5-(4-(3-(2,4-Difluorophenyl)ureido)phenyl)thiazol-2-yl)adamantane-1-carboxylic acid

The compound of example 504 was prepared analogous to the compound of example 7 by hydrolysis of the compound of example 503. Yield: 82%; ¹H NMR (DMSO-d₆, 300 MHz): δ 12.19 (s, 1H), 9.19 (s, 1H), 8.54 (s, 1H), 8.06 (m, 1H), 7.94 (s, 1H), 7.55 (m, 4H), 7.32 (m 1H), 7.05 (t, 1H), 2.14 (s, 2H), 2.04 (s, 2H), 1.93 (s, 4H), 1.81 (s, 4H), 1.68 (s, 1H), 1.20 (s, 1H); MS: m/z 510 (M+1).

### Example 505

### Methyl 3-(5-(4-(3-(2,6-difluorophenyl)ureido)phenyl)thiazol-2-yl)adamantine-1-carboxylate

The compound of example 505 was prepared analogous to the compound of example 6 by reaction of the compound of example 498 with 2,6-difluoro-1-isocyanatobenzene.

Yield: 96%; ¹H NMR (DMSO-d₆, 300 MHz): δ 9.09 (s, 1H), 8.15 (s, 1H), 7.94 (s, 1H), 7.54 (m, 4H), 7.29 (m, 1H), 7.16 (m, 2H), 3.59 (s, 3H), 2.15 (s, 2H), 2.07 (s, 2H), 1.94 (s, 4H), 1.83 (s, 4H), 1.68 (s, 2H); MS: m/z 522 (M−1).

### Example 506

### 3-(5-(4-(3-(2,6-Difluorophenyl)ureido)phenyl)thiazol-2-yl)adamantane-1-carboxylic acid

The compound of example 506 was prepared analogous to the compound of example 7 by hydrolysis of the compound of example 505. Yield: 94%; ¹H NMR (DMSO-d₆, 300 MHz): δ 12.19 (s, 1H), 9.13 (s, 1H), 8.17 (s, 1H), 7.94 (s, 1H), 7.54 (m, 4H), 7.32 (m, 1H), 7.16 (m 1H), 2.14 (s, 2H), 2.04 (s, 2H), 1.93 (s, 4H), 1.81 (s, 4H), 1.68 (s, 2H); MS: m/z 510 (M+1).

### Example 507

### Methyl 3-(5-(4-(3-(2,4,5-trifluorophenyl)ureido)phenyl)thiazol-2-yl)adamantane-1-carboxylate

The compound of example 507 was prepared analogous to the compound of example by reaction of the compound of example 498 with 2,4,5-trifluoro-1-isocyanatobenzene. Yield: 84%; ¹H NMR (DMSO-d₆, 300 MHz): δ 9.21 (s, 1H), 8.73 (s, 1H), 8.20 (m, 1H), 7.96 (s, 1H), 7.63 (m, 1H), 7.57 (d, 2H), 7.50 (d, 2H), 3.60 (s, 3H), 2.16 (s, 2H), 2.08 (s, 2H), 1.97 (s, 4H), 1.84 (s, 4H), 1.69 (s, 2H); MS: m/z 542 (M+1).

### Example 508

### 3-(5-(4-(3-(2,4,5-Trifluorophenyl)ureido)phenyl)thiazol-2-yl)adamantane-1-carboxylic acid

The compound of example 508 was prepared analogous to the compound of example 7 by hydrolysis of the compound of example 507. Yield: 89%; ¹H NMR (DMSO-d₆, 300 MHz): δ 12.18 (s, 1H), 9.25 (s, 1H), 8.75 (s, 1H), 8.23 (m, 1H), 7.96 (s, 1H), 7.67 (m, 1H), 7.57 (d, 2H), 7.50 (d, 2H), 2.16 (s, 2H), 2.05 (s, 2H), 1.94 (s, 4H), 1.82 (s, 4H), 1.69 (s, 2H); MS: m/z 528 (M+1).

### Example 509

### Methyl 3-(5-(4-(3-(2,3,4-trifluorophenyl)ureido)phenyl)thiazol-2-yl)adamantane-1-carboxylate

The compound of example 509 was prepared analogous to the compound of example by reaction of the compound of example 498 with 2,3,4-trifluoro-1-isocyanatobenzene. Yield: 90%; ¹H NMR (DMSO-d₆, 300 MHz): δ 9.18 (s, 1H), 8.70 (s, 1H), 7.96 (s, 1H), 7.86 (m, 1H), 7.56 (m, 4H), 7.28 (m, 1H), 3.59 (s, 3H), 2.16 (s, 2H), 2.08 (s, 2H), 1.89 (s, 4H), 1.80 (s, 4H), 1.69 (s, 2H); MS: m/z 542 (M+1).

### Example 510

### 3-(5-(4-(3-(2,3,4-Trifluorophenyl)ureido)phenyl)thiazol-2-yl)adamantane-1-carboxylic acid

The compound of example 510 was prepared analogous to the compound of example 7 by hydrolysis of the compound of example 509. Yield: 89%; ¹H NMR (DMSO-d₆, 300 MHz): δ 12.18 (s, 1H), 9.25 (s, 1H), 8.75 (s, 1H), 8.23 (m, 1H), 7.96 (s, 1H), 7.67 (m, 1H), 7.57 (d, 2H), 7.50 (d, 2H), 2.16 (s, 2H), 2.05 (s, 2H), 1.94 (s, 4H), 1.82 (s, 4H), 1.69 (s, 2H); MS: m/z 528 (M+1).

### Example 511

### Methyl 3-(5-(4-(3-(3,5-difluorophenyl)ureido)phenyl)thiazol-2-yl)adamantane-1-carboxylate

The compound of example 511 was prepared analogous to the compound of example 6 by reaction of the compound of example 498 with 3,5-difluoro-1-isocyanatobenzene.

Yield: 90%; ¹H NMR (DMSO-d₆, 300 MHz): δ 9.11 (s, 1H), 8.90 (s, 1H), 7.96 (s, 1H), 7.56 (m, 4H), 7.20 (m, 2H), 6.79 (m, 1H), 3.59 (s, 3H), 2.16 (s, 2H), 2.08 (s, 2H), 1.95 (s, 4H), 1.84 (s, 4H), 1.69 (s, 2H); MS: m/z 524 (M+1).

### Example 512

### 3-(5-(4-(3-(3,5-Difluorophenyl)ureido)phenyl)thiazol-2-yl)adamantane-1-carboxylic acid

The compound of example 512 was prepared analogous to the compound of example 7 by hydrolysis of the compound of example 511. Yield: 89%; ¹H NMR (DMSO-d₆, 300 MHz): δ 12.20 (s, 1H), 9.34 (s, 1H), 9.16 (s, 1H), 7.96 (s, 1H), 7.56 (d, 2H), 7.51 (d, 2H), 7.19 (d, 2H), 6.78 (m, 1H), 2.16 (s, 2H), 2.05 (s, 2H), 1.94 (s, 4H), 1.82 (s, 4H), 1.69 (s, 2H); MS: m/z 510 (M+1).

### Example 513

### Methyl 3-(5-(4-(3-(3-(trifluoromethyl)phenyl)ureido)phenyl)thiazol-2-yl)adamantane-1-carboxylate

The compound of example 513 was prepared analogous to the compound of example by reaction of the compound of example 498 with 1-isocyanato-3-trifluoromethylbenzene. Yield: 93%; ¹H NMR (DMSO-d₆, 300 MHz): δ 9.07 (s, 1H), 8.94 (s, 1H), 8.00 (s, 1H), 7.96 (s, 1H), 7.58 (m, 5H), 7.31 (m, 1H), 3.60 (s, 3H), 2.16 (s, 2H), 2.08 (s, 2H), 1.95 (s, 4H), 1.84 (s, 4H), 1.69 (s, 2H); MS: m/z 556 (M+1).

### Example 514

### 3-(5-(4-(3-(3-(Trifluoromethyl)phenyl)ureido)phenyl)thiazol-2-yl)adamantane-1-carboxylic acid

The compound of example 514 was prepared analogous to the compound of example 7 by hydrolysis of the compound of example 513. Yield: 90%; ¹H NMR (DMSO-d₆, 300 MHz): δ 12.17 (s, 1H), 9.10 (s, 1H), 8.97 (s, 1H), 8.00 (s, 1H), 7.96 (s, 1H), 7.58 (m, 6H), 7.31 (d, 1H), 2.16 (s, 2H), 2.05 (s, 2H), 1.90 (s, 4H), 1.78 (s, 4H), 1.69 (s, 2H); MS: m/z 542 (M+1).

### Example 515

### 3-(t-Butoxycarbonyl)amino)propanoic acid

To a suspension of 3-aminopropanoic acid (10 g, 112 mmol) in acetonitrile (100 mL) and water (150 mL) was added sodium bicarbonate (20.74 g, 247 mmol) and cooled to 0° C. To this reaction mixture, a solution of BOC-anhydride (28.7 mL, 123 mmol) in acetonitrile (50 mL) was added dropwise over 20 min and stirred for 16 h. Ethyl acetate (200 mL) was added and pH was adjusted to 4-5 by addition of NaH₂PO₄.2H₂O. The product was extracted with ethyl acetate (3×500 mL), dried over sodium sulfate and evaporated to dryness to afford the title compound. Yield: 17.7 g (83%); ¹H NMR (DMSO-d₆, 300 MHz): δ 12.15 (bs, 1H), 6.78 (s, 1H), 3.12-3.06 (t, 2H), 3.34-3.29 (t, 2H), 1.34 (s, 9H); MS: m/z 188.1 (M−1).

### Example 516

### t-Butyl (3-((2-(4-nitrophenyl)-2-oxoethyl)amino)-3-oxopropyl)carbamate

To a solution of the compound of example 515 (17.47 g, 92 mmol) in DMF (400 mL) was added HATU (38.6 g, 102 mmol), compound of example 2 (20 g, 92 mmol) and TEA (25.7 mL, 185 mmol). The mixture was stirred at room temperature for 4 h. The organic solvent was removed to obtain a residue which was purified by column chromatography (silica gel, 20% acetone in chloroform) to obtain a solid, which was crystallized in chloroform:petroleum ether to afford the title compound. Yield: 21.3 g (66%) ¹H NMR (DMSO-d₆, 300 MHz): δ 8.34-8.31 (m, 3H), 8.19-8.16 (d, 2H), 6.74-6.70 (t, 1H), 4.63-4.61 (d, 2H), 3.12-3.07 (m, 2H), 2.35-2.30 (t, 2H), 1.35 (s, 9H); MS: m/z 352.1 (M+1).

### Example 517

### t-Butyl (2-(5-(4-nitrophenyl)thiazol-2-yl)ethyl)carbamate

To a solution of the compound of example 516 (48 g, 137 mmol) in ethyl acetate (960 mL) was added Lawesson's reagent (44.2 g, 109 mmol) and heated to reflux for 30 min. The reaction mass was adsorbed onto silica and purified by flash column chromatography (silica gel, 40% ethyl acetate in petroleum ether) to obtain a solid, which was stirred in ethanol to afford the title compound. Yield: 19.1 g (40%); ¹H NMR (DMSO-d₆, 300 MHz): δ 8.34 (s, 1H), 8.27-8.24 (d, 2H), 7.90-7.88 (d, 2H), 7.03-7.00 (t, 1H), 3.34-3.28 (m, 2H), 3.13-3.09 (m, 2H), 1.34 (s, 9H); MS: m/z 350.1 (M+1).

### Example 518

### 2-(5-(4-Nitrophenyl)thiazol-2-yl)ethanamine hydrochloride

To the compound of example 517 (18 g, 51.5 mmol) in methanol (360 mL) was added 4M HCl in 1,4-dioxane (129 mL, 515 mmol) and stirred for 16 h at room temperature. The solvent was removed to obtain a solid, which was stirred in diethyl ether, filtered, and dried to afford the title compound. Yield: 14 g (95%); ¹H NMR (DMSO-d₆, 300 MHz): δ 8.41 (s, 1H), 8.30-8.27 (d, 2H), 8.22 (bs, 2H), 7.96-7.93 (d, 2H), 3.40-3.38 (m, 2H), 3.27-3.25 (m, 2H); MS: m/z 250 (M+1).

### Example 519

### 1,1,1-Trifluoro-N-(2-(5-(4-nitrophenyl)thiazol-2-yl)ethyl)methanesulfonamide

To a suspension of the compound of example 518 (1.5 g, 5.25 mmol) in dichloromethane (30 mL) was added triflic anhydride (1.064 mL, 6.30 mmol) followed by triethylamine (2.195 mL, 15.75 mmol) and stirred at room temperature for 24 h. The solvent was evaporated to obtain a residue, which was purified by column chromatography (silica gel, 40% ethyl acetate in chloroform) to obtain a solid, which was crystallized in chloroform:petroleum ether to afford the title compound. Yield: 1.37 g (68%); ¹H NMR (DMSO-d₆, 300 MHz): δ 9.68 (bs, 1H), 8.42 (s, 1H), 8.30-8.27 (d, 2H), 7.96-7.93 (d, 2H), 3.62-3.58 (m, 2H), 3.30-3.26 (m, 2H); MS: m/z 382 (M+1).

### Example 520

### N-(2-(5-(4-Aminophenyl)thiazol-2-yl)ethyl)-1,1,1-trifluoromethane sulfonamide

The compound of example 520 was prepared analogous to the compound of example 378 by reduction of compound of example 519. Yield: 63%; ¹H NMR (DMSO-d₆, 300 MHz): δ 9.65 (bs, 1H), 7.81 (s, 1H), 7.29-8.26 (d, 2H), 6.61-6.58 (d, 2H), 5.41 (bs, 2H), 3.57-3.52 (m, 2H), 3.19-3.14 (m, 2H); MS: m/z 352 (M+1).

### Example 521

### N-(2-(5-(4-(3-(2-Chlorophenyl)ureido)phenyl)thiazol-2-yl)ethyl)-1,1,1-trifluoro methanesulfonamide

The compound of example 521 was prepared analogous to the compound of example 6 by reaction of the compound of example 520 with 2-chloro-1-isocyanatobenzene.

Yield: 85%; ¹H NMR (DMSO-d₆, 300 MHz): δ 9.66 (bs, 1H), 9.59 (s, 1H), 8.35 (s, 1H), 8.18-8.15 (dd, 1H), 8.04 (s, 1H), 7.60-7.52 (dd, 4H), 7.49-7.42 (dd, 1H), 7.34-7.28 (m, 1H), 7.07-7.02 (m, 1H), 3.60-3.55 (t, 2H), 3.24-3.19 (t, 2H); MS: m/z 505 (M+1).

### Example 522

### 1,1,1-Trifluoro-N-(2-(5-(4-(3-(2-fluorophenyl)ureido)phenyl)thiazol-2-yl)ethyl)methanesulfonamide

The compound of example 522 was prepared analogous to the compound of example 6 by reaction of the compound of example 520 with 2-fluoro-1-isocyanatobenzene.

Yield: 79%; ¹H NMR (DMSO-d₆, 300 MHz): δ 9.66 (s, 1H), 9.25 (s, 1H), 8.59 (d, 1H), 8.18-8.13 (dd, 1H), 8.03 (s, 1H), 7.59-7.51 (dd, 4H), 7.28-7.24 (m, 1H), 7.22-7.13 (m, 1H), 7.06-7.02 (m, 1H), 3.60-3.55 (t, 2H), 3.24-3.19 (t, 2H); MS: m/z 489.1 (M+1).

### Example 523

### N-(2-(5-(4-(3-(3,5-Difluorophenyl)ureido)phenyl)thiazol-2-yl)ethyl)-1,1,1-trifluoromethanesulfonamide

The compound of example 523 was prepared analogous to the compound of example 6 by reaction of the compound of example 520 with 3,5-difluoro-1-isocyanatobenzene.

Yield: 83%; ¹H NMR (DMSO-d₆, 300 MHz): δ 9.66 (bs, 1H), 9.13 (s, 1H), 9.03 (s, 1H), 8.03 (s, 1H), 7.59-7.51 (dd, 4H), 7.22-7.19 (m, 2H), 6.84-6.77 (m, 1H), 3.60-3.55 (t, 2H), 3.24-3.19 (t, 2H); MS: m/z 507.1 (M+1).

### Example 524

### 1,1,1-Trifluoro-N-(2-(5-(4-(3-(2,4,5-trifluorophenyl)ureido)phenyl)thiazol-2-yl)ethyl)methanesulfonamide

The compound of example 524 was prepared analogous to the compound of example by reaction of the compound of example 520 with 2,4,5-trifluoro-1-isocyanatobenzene. Yield: 92%; ¹H NMR (DMSO-d₆, 300 MHz) δ 9.66 (bs, 1H), 9.25 (s, 1H), 8.75 (s, 1H), 8.25-8.15 (m, 1H), 7.39 (s, 1H), 7.69-7.65 (m, 1H), 7.63-7.51 (dd, 4H), 3.60-3.55 (t, 2H), 3.24-3.20 (t, 2H); MS: m/z 525.1 (M+1).

### Example 525

### 1,1,1-Trifluoro-N-(2-(5-(4-(3-(2,4,6-trifluorophenyl)ureido)phenyl)thiazol-2-yl) ethyl)methanesulfonamide

The compound of example 525 was prepared analogous to the compound of example by reaction of the compound of example 520 with 2,4,6-trifluoro-1-isocyanatobenzene. Yield: 82%; ¹H NMR (DMSO-d₆, 300 MHz): δ 9.66 (bs, 1H), 9.17 (s, 1H), 8.08 (s, 1H), 8.02 (s, 1H), 7.57-7.50 (dd, 4H), 7.31-7.23 (m, 3H), 3.59-3.55 (t, 2H), 3.24-3.19 (t, 2H); MS: m/z 525.1 (M+1).

### Example 526

### 1,1,1-Trifluoro-N-(2-(5-(4-(3-(4-(trifluoromethyl)phenyl)ureido)phenyl)thiazol-2-yl)ethylmethanesulfonamide

The compound of example 526 was prepared analogous to the compound of example 6 by reaction of the compound of example 520 with 1-isocyanato-4-trifluoromethyl benzene. Yield: 75%; ¹H NMR (DMSO-d₆, 300 MHz): δ 9.66 (bs, 1H), 9.14 (s, 1H), 8.99 (s, 1H), 8.03 (s, 1H), 7.66-7.65 (dd, 4H), 7.56-7.55 (dd, 4H), 3.62-3.53 (t, 2H), 3.24-3.19 (t, 2H); MS: m/z 539 (M+1).

### Example 527

### 1,1,1-Trifluoro-N-(2-(5-(4-(3-phenyl ureido)phenyl)thiazol-2-yl)ethyl)methane sulfonamide

The compound of example 527 was prepared analogous to the compound of example 6 by reaction of the compound of example 520 with isocyanato benzene. Yield: 51%;

¹H NMR (DMSO-d₆, 300 MHz): δ 9.65 (bs, 1H), 8.85 (s, 1H), 8.70 (s, 1H), 8.02 (s, 1H), 7.54-7.53 (dd, 4H), 7.47-7.44 (m, 2H), 7.31-7.26 (m, 2H), 6.98 (m, 1H), 3.57-3.54 (t, 2H), 3.23-3.19 (t, 2H); MS: m/z 471.1 (M+1).

### Example 528

### N-(2-(5-(4-(3-Cyclohexylureido)phenyl)thiazol-2-yl)ethyl)-1,1,1-trifluoro methanesulfonamide

The compound of example 528 was prepared analogous to the compound of example 6 by reaction of the compound of example 520 with cyclohexyl isocyanate. Yield: 73%;

¹H NMR (DMSO-d₆, 300 MHz): δ 9.66 (bs, 1H), 8.48 (s, 1H), 7.97 (s, 1H), 7.50-7.41 (dd, 4H), 6.13-6.11 (d, 1H), 3.58-3.53 (t, 2H), 3.46-3.43 (m, 1H), 3.24-3.17 (t, 2H), 1.85-1.78 (m, 2H), 1.72-1.68 (m, 2H), 1.58-1.52 (m, 1H), 1.33-1.14 (m, 5H); MS: m/z 477.1 (M+1).

### Example 529

### 2-Chloro-N-(4-(2-(2-(trifluoromethylsulfonamido)ethyl)thiazol-5-yl)phenyl)benzamide

To a solution of the compound of example 520 (70 mg, 0.199 mmol) in dichloromethane (2.8 mL) was added triethylamine (0.069 mL, 0.498 mmol) followed by 2-chlorobenzoyl chloride (0.030 mL, 0.239 mmol) and stirred at room temperature for 24 h. The solvent was evaporated to obtain a residue, which was crystallized in ethyl acetate:petroleum ether and filtered to afford the title compound. Yield: 74 mg (76%);

¹H NMR (DMSO-d₆, 300 MHz) δ 10.66 (s, 1H), 8.07 (s, 1H), 7.81-7.78 (d, 2H), 7.71-7.68 (m, 1H), 7.67-7.57 (m, 4H), 7.55-7.46 (m, 2H), 4.30-4.25 (t, 2H), 3.42-3.38 (t, 2H); MS: m/z 490 (M+1).

### Example 530

### N-(4-(2-(2-(Trifluoromethylsulfonamido)ethyl)thiazol-5-yl)phenyl)cyclohexane carboxamide

The compound of example 530 was prepared analogous to the compound of example 529 by reaction of the compound of example 520 with cyclohexanecarbonyl chloride.

Yield: 27%; ¹H NMR (DMSO-d₆, 300 MHz) δ 9.97 (s, 1H), 8.12 (s, 1H), 7.70-7.58 (dd, 4H), 6.98-6.89 (m, 1H), 4.30-4.25 (t, 2H), 3.40-3.36 (t, 2H) 3.44-3.40 (m, 1H), 2.33 (t, 1H), 1.88-1.62 (m, 5H), 1.48-1.15 (m, 4H); MS: m/z 462 (M+1).

### Example 531

### 4-(Trifluoromethyl)-N-(4-(2-(2-(trifluoromethylsulfonamido)ethyl)thiazol-5-yl) phenyl)benzamide

The compound of example 531 was prepared analogous to the compound of example 529 by reaction of the compound of example 520 with 4-trifluoromethylbenzoyl chloride.

Yield: 42%; ¹H NMR (DMSO-d₆, 300 MHz) δ 10.61 (s, 1H), 9.67 (bs, 1H), 8.17-8.15 (d, 2H), 8.09 (s, 1H), 7.98-7.86 (m, 4H), 7.67-7.61 (m, 2H), 3.57-3.55 (t, 2H), 3.25-3.22 (t, 2H); MS: m/z 524 (M+1).

### Example 532

### N-(4-(2-(2-(Trifluoromethylsulfonamido)ethyl)thiazol-5-yl)phenyl)benzamide

The compound of example 532 was prepared analogous to the compound of example 529 by reaction of the compound of example 520 with benzoyl chloride. Yield: 28%; ¹H NMR (DMSO-d₆, 300 MHz) δ 10.39 (s, 1H), 8.02 (s, 1H), 7.98-7.95 (d, 2H), 7.88-7.85 (d, 2H), 7.77-7.74 (d, 2H), 7.69-7.54 (m, 4H), 4.41-4.32 (t, 2H), 3.44-3.39 (t, 2H); MS: m/z 456.1 (M+1).

### Example 533

### 2-Phenyl-5-(trifluoromethyl)-N-(4-(2-(2-(trifluoromethylsulfonamido)ethyl)thiazol-5-yl)phenyl)oxazole-4-carboxamide

The compound of example 533 was prepared analogous to the compound of example 529 by reaction of the compound of example 520 with 2-phenyl-5-(trifluoromethyl)oxazole-4-carbonyl chloride. Yield: 59%; ¹H NMR (DMSO-d₆, 300 MHz): δ 10.73 (s, 1H), 9.68 (bs, 1H), 8.18-8.15 (m, 2H), 8.12 (s, 1H), 7.94-7.91 (d, 2H), 7.69-7.66 (m, 5H), 3.60-3.56 (t, 2H), 3.25-3.21 (t, 2H); MS: m/z 591 (M+1).

### Example 534

### 1,1,1-Trifluoro-N-(2-(5-(4-(3-(2-fluorophenyl)thioureido)phenyl)thiazol-2-yl) ethyl)methanesulfonamide

The compound of example 534 was prepared analogous to the compound of example 6 by reaction of the compound of example 520 with 2-fluoro-1-isothiocyanatobenzene.

Yield: 84%; ¹H NMR (DMSO-d₆, 300 MHz) δ 10.10 (s, 1H), 9.67 (bs, 1H), 9.57 (s, 1H), 8.09 (s, 1H), 7.64-7.57 (m, 5H), 7.29-7.25 (m, 2H), 7.22-7.16 (m, 1H), 3.59-3.55 (t, 2H), 3.24-3.20 (t, 2H); MS: m/z 505.1 (M+1).

### Example 535

### 1,1,1-Trifluoro-N-(2-(5-(4-(3-(2-fluorophenyl)guanidino)phenyl)thiazol-2-yl)ethyl)methanesulfonamide

To a solution of the compound of example 534 (150 mg, 0.297 mmol) in 7N methanolic ammonia (4.25 mL, 29.7 mmol), was added mercuric oxide yellow (161 mg, 0.743 mmol) and the reaction mixture was stirred at room temperature for 2 h. After completion of the reaction, the solvent was removed and chloroform was added. The residue was filtered through Celite®, filtrate was concentrated and purified by flash chromatography (silica gel, 60% ethyl acetate in chloroform) to afford the title compound. Yield: 85 mg (57%); ¹H NMR (DMSO-d₆, 300 MHz): δ 9.01 (bs, 2H), 7.97 (s, 1H), 7.54-7.48 (m, 5H), 7.19-7.00 (m, 3H), 5.78 (bs, 2H), 3.61-3.55 (t, 2H), 3.21-3.17 (t, 2H); MS: m/z 488.1 (M+1).

### Example 536

### 1,1,1-Trifluoro-N-(2-(5-(4-(3-(2-fluorophenyl)-2-methylguanidino)phenyl)thiazol-2-yl)ethyl)methanesulfonamide

The compound of example 536 was prepared analogous to the compound of example 535 by reaction of the compound of example 534 with methanamine. Yield: 67%; ¹H NMR (DMSO-d₆, 300 MHz): δ 10.62 (bs, 1H), 9.30 (bs, 1H), 7.96 (s, 1H), 7.51-7.48 (d, 2H), 7.23-7.20 (d, 2H), 7.15-6.98 (m, 4H), 6.63 (bs, 1H), 3.48-3.44 (t, 2H), 3.15-3.10 (t, 2H), 2.78 (s, 3H); MS: m/z 502.1 (M+1).

### Example 537

### N-(2-(5-(4-(2-Cyano-3-(2-fluorophenyl)guanidino)phenyl)thiazol-2-yl)ethyl)-1,1,1-trifluoromethanesulfonamide

The compound of example 536 was prepared analogous to the compound of example 535 by reaction of the compound of example 534 with cyanamide. Yield: 75%; ¹H NMR (DMSO-d₆, 300 MHz): δ 9.66 (bs, 1H), 9.58 (s, 1H), 9.43 (s, 1H), 8.08 (s, 1H), 7.63-7.61 (d, 2H), 7.38-7.35 (d, 2H), 7.33-7.25 (m, 3H), 7.23-7.17 (m, 1H), 3.57-3.55 (t, 2H), 3.24-3.20 (t, 2H); MS: m/z 513.1 (M+1).

### Example 538

### t-Butyl (2-((2-(4-nitrophenyl)-2-oxoethyl)amino)-2-oxoethyl)carbamate

The compound of example 538 was prepared analogous to the compound of example 516 by reaction of the compound of example 2 with 2-(tert-butoxycarbonylamino)acetic acid. Yield: 79%; ¹H NMR (DMSO-d₆, 300 MHz): δ 8.36-8.32 (d, 2H), 8.22-8.19 (m, 3H), 7.09-7.05 (t, 1H), 4.69-4.67 (d, 2H), 3.63-3.61 (m, 2H), 1.38 (s, 9H); MS: m/z 338.3 (M+1).

### Example 539

### t-Butyl((5-(4-nitrophenyl)thiazol-2-yl)methyl)carbamate

The compound of example 539 was prepared analogous to the compound of example 517 by reaction of the compound of example 538 with Lawesson's reagent. Yield: 61%; ¹H NMR (DMSO-d₆, 300 MHz): δ 8.36 (s, 1H), 8.31-8.25 (d, 2H), 7.95-7.89 (d, 2H), 7.87-7.85 (t, 1H), 4.43-4.41 (d, 2H), 1.42 (s, 9H); MS: m/z 336.1 (M+1).

### Example 540

### (5-(4-Nitrophenyl)thiazol-2-yl)methanamine hydrochloride

The compound of example 540 was prepared analogous to the compound of example 518 by reaction of the compound of example 539 with HCl. Yield: 77%; ¹H NMR (DMSO-d₆, 300 MHz): δ 8.33 (s, 1H), 8.27-8.24 (d, 2H), 7.94-7.91 (d, 2H), 4.02 (d, 2H), 2.42 (bs, 2H); MS: m/z 236.1 (M+1).

### Example 541

### 1,1,1-Trifluoro-N-((5-(4-nitrophenyl)thiazol-2-yl)methyl)methanesulfonamide

The compound of example 541 was prepared analogous to the compound of example 519 by reaction of the compound of example 540 with triflic anhydride. Yield: 21%; ¹H NMR (DMSO-d₆, 300 MHz): δ 9.45 (bs, 1H), 8.39 (s, 1H), 8.34-8.31 (d, 2H), 7.92-7.89 (d, 2H), 4.42-4.40 (d, 2H); MS: m/z 368.1 (M+1).

### Example 542

### N-((5-(4-Aminophenyl)thiazol-2-yl)methyl)-1,1,1-trifluoromethane sulfonamide

The compound of example 542 was prepared analogous to the compound of example 378 by reduction of compound of example 541. Yield: 51%; ¹H NMR (DMSO-d₆, 300 MHz): δ 10.16 (bs, 1H), 7.86 (s, 1H), 7.33-7.30 (d, 2H), 6.61-6.58 (d, 2H), 5.61 (bs, 2H), 4.63 (d, 2H); MS: m/z 338 (M+1).

### Example 543

### N-((5-(4-(3-(2-Chlorophenyl)ureido)phenyl)thiazol-2-yl)methyl)-1,1,1-trifluoro methanesulfonamide

The compound of example 543 was prepared analogous to the compound of example 6 by reaction of the compound of example 542 with 2-chloro-1-isocyanatobenzene.

Yield: 80%; ¹H NMR (DMSO-d₆, 300 MHz): δ 10.49 (bs, 1H), 9.60 (s, 1H), 8.35 (s, 1H), 8.18-8.15 (dd, 1H), 8.08 (s, 1H), 7.64-7.53 (dd, 4H), 7.48-7.46 (dd, 1H), 7.34-7.29 (m, 1H), 7.07-7.02 (m, 1H), 4.75 (s, 2H); MS: m/z 491 (M+1).

### Example 544

### 1,1,1-Trifluoro-N-((5-(4-(3-(2-fluorophenyl)ureido)phenyl)thiazol-2-yl)methyl)methanesulfonamide

The compound of example 543 was prepared analogous to the compound of example 6 by reaction of the compound of example 542 with 2-fluoro-1-isocyanatobenzene.

Yield: 64%; ¹H NMR (DMSO-d₆, 300 MHz): δ 10.48 (bs, 1H), 9.26 (s, 1H), 8.59 (s, 1H), 8.17-8.12 (m, 1H), 8.08 (s, 1H), 7.63-7.52 (dd, 4H), 7.28-7.21 (m, 1H), 7.18-7.13 (m, 1H), 7.05-7.01 (m, 1H), 4.75 (s, 2H); MS: m/z 475 (M+1).

### Example 545

### N-((5-(4-(3-(3,5-Difluorophenyl)ureido)phenyl)thiazol-2-yl)methyl)-1,1,1-trifluoromethanesulfonamide

The compound of example 545 was prepared analogous to the compound of example 6 by reaction of the compound of example 542 with 3,5-difluoro-1-isocyanatobenzene.

Yield: 70%; ¹H NMR (DMSO-d₆, 300 MHz): δ 10.49 (bs, 1H), 9.14 (s, 1H), 9.06 (s, 1H), 8.08 (s, 1H), 7.63-7.52 (dd, 4H), 7.21-7.19 (m, 2H), 6.84-6.78 (m, 1H), 4.75 (s, 2H); MS: m/z 493 (M+1).

### Example 546

### 1,1,1-Trifluoro-N-((5-(4-(3-(2,4,5-trifluorophenyl)ureido)phenyl)thiazol-2-yl) methyl)methanesulfonamide

The compound of example 546 was prepared analogous to the compound of example by reaction of the compound of example 542 with 2,4,5-trifluoro-1-isocyanatobenzene. Yield: 72%; ¹H NMR (DMSO-d₆, 300 MHz): δ 10.49 (bs, 1H), 9.27 (s, 1H), 8.76 (s, 1H), 8.24-8.15 (m, 1H), 8.09 (s, 1H), 7.69-7.67 (m, 1H), 7.63-7.61 (d, 2H), 7.54-7.51 (m, 2H), 4.69 (s, 2H); MS: m/z 511 (M+1).

### Example 547

### 1,1,1-Trifluoro-N-((5-(4-(3-(2,4,6-trifluorophenyl)ureido)phenyl)thiazol-2-yl) methyl)methanesulfonamide

The compound of example 547 was prepared analogous to the compound of example 6 by reaction of the compound of example 542 with 2,4,6-trifluoro-1-isocyanatobenzene. Yield: 93%; ¹H NMR (DMSO-d₆, 300 MHz): δ 10.49 (bs, 1H), 9.19 (s, 1H), 8.32 (s, 1H), 8.07 (s, 1H), 7.61-7.51 (dd, 4H), 7.31-7.23 (m, 2H), 4.75 (s, 2H); MS: m/z 511 (M+1).

### Example 548

### N-((5-(4-(3-Cyclohexylureido)phenyl)thiazol-2-yl)methyl)-1,1,1-trifluoro methanesulfonamide

The compound of example 548 was prepared analogous to the compound of example 6 by reaction of the compound of example 542 with cyclohexyl isocyanate. Yield: 36%;

¹H NMR (DMSO-d₆, 300 MHz): δ 10.47 (bs, 1H), 8.50 (s, 1H), 8.03 (s, 1H), 7.54-7.43 (dd, 4H), 6.14-6.11 (m, 1H), 4.67 (s, 2H), 3.46 (m, 1H), 1.79 (m, 2H), 1.64 (m, 2H), 1.52 (m, 1H), 1.33-1.15 (m, 5H); MS: m/z 463.1 (M+1).

### Example 549

### 1,1,1-Trifluoro-N-((5-(4-(3-(4-(trifluoromethyl)phenyl)ureido)phenyl)thiazol-2-yl)methyl)methanesulfonamide

The compound of example 549 was prepared analogous to the compound of example by reaction of the compound of example 542 with 1-isocyanato-4-trifluoromethylbenzene. Yield: 59%; ¹H NMR (DMSO-d₆, 300 MHz): δ 10.49 (bs, 1H), 9.15 (s, 1H), 9.01 (s, 1H), 8.08 (s, 1H), 7.69-7.63 (m, 4H), 7.60-7.53 (m, 4H), 4.75 (s, 2H); MS: m/z 525 (M+1).

### Example 550

### 1,1,1-Trifluoro-N-((5-(4-(3-phenylureido)phenyl)thiazol-2-yl)methyl)methane sulfonamide

The compound of example 550 was prepared analogous to the compound of example 6 by reaction of the compound of example 542 with isocyanatobenzene. Yield: 76%;

¹H NMR (DMSO-d₆, 300 MHz): δ 10.48 (bs, 1H), 8.87 (s, 1H), 8.71 (s, 1H), 8.07 (s, 1H), 7.61-7.52 (m, 4H), 7.47-7.42 (d, 2H), 7.31-7.26 (m, 2H), 7.00-6.95 (m, 1H), 4.69 (s, 2H); MS: m/z 457 (M+1).

### Example 551

### 2-Chloro-N-(4-(2-((trifluoromethylsulfonamido)methyl)thiazol-5-yl-phenyl)benzamide

The compound of example 551 was prepared analogous to the compound of example 529 by reaction of the compound of example 542 with 2-chlorobenzoyl chloride. Yield: 85%; ¹H NMR (DMSO-d₆, 300 MHz): δ 10.68 (s, 1H), 8.15 (s, 1H), 7.81-7.78 (d, 2H), 7.66-7.63 (d, 2H), 7.61-7.57 (m, 4H), 7.55-7.46 (m, 1H), 5.40 (s, 2H); MS: m/z 476 (M+1).

### Example 552

### 4-(Trifluoromethyl)-N-(4-(2-((trifluoromethylsulfonamido)methyl)thiazol-5-yl) phenyl)benzamide

The compound of example 552 was prepared analogous to the compound of example 529 by reaction of the compound of example 542 with 4-trifluoromethylbenzoyl chloride.

Yield: 59%; ¹H NMR (DMSO-d₆, 300 MHz): δ 10.62 (s, 1H), 10.50 (bs, 1H), 8.18-8.14 (m, 3H), 7.95-7.87 (dd, 4H), 7.71-7.69 (d, 2H), 4.70 (s, 2H); MS: m/z 510 (M+1).

### Example 553

### N-(4-(2-((Trifluoromethylsulfonamido)methyl)thiazol-5-yl)phenyl)benzene sulfonamide

To a solution of the compound of example 542 (70 mg, 0.208 mmol) in dichloromethane (2.8 mL) was added triethylamine (0.072 mL, 0.519 mmol) followed by benzenesulfonyl chloride (0.029 mL, 0.228 mmol) and stirred at room temperature for 24 h. The solvent was evaporated to obtain a residue, which was crystallized in ethyl acetate: petroleum ether and filtered to afford the title compound. Yield: 50 mg (50%);

¹H NMR (DMSO-d₆, 300 MHz): δ 10.53 (s, 1H), 10.47 (bs, 1H), 8.04 (s, 1H), 7.80-7.74 (d, 2H), 7.65-7.53 (m, 5H), 7.17-7.14 (d, 2H), 4.67 (s, 2H); MS: m/z 476 (M−1).

### Example 554

### 4-(Trifluoromethyl)-N-(4-(2-((trifluoromethylsulfonamido)methyl)thiazol-5-yl) phenyl)benzenesulfonamide

The compound of example 554 was prepared analogous to the compound of example 553 by reaction of the compound of example 542 with 4-trifluoromethyl benzenesulfonyl chloride. Yield: 46%; ¹H NMR (DMSO-d₆, 300 MHz): δ 10.75 (s, 1H), 10.48 (bs, 1H), 8.06 (s, 1H), 7.98 (m, 4H), 7.59-7.56 (d, 2H), 7.18-7.15 (d, 2H), 4.67 (s, 2H); MS: m/z 546 (M+1).

### Example 555

### N-(4-(2-((Trifluoromethylsulfonamido)methyl)thiazol-5-yl)phenyl)cyclohexane sulfonamide

The compound of example 555 was prepared analogous to the compound of example 553 by reaction of the compound of example 542 with cyclohexanesulfonyl chloride.

Yield: 30%; ¹H NMR (DMSO-d₆, 300 MHz): δ 10.49 (bs, 1H), 9.98 (s, 1H), 8.08 (s, 1H), 7.63-7.60 (d, 2H), 7.29-7.26 (d, 2H), 4.69 (s, 2H), 3.03 (t, 1H), 2.03-2.00 (m, 2H), 1.69-1.79 (m, 2H), 1.59 (m, 1H), 1.43-1.29 (m, 2H), 1.23-1.15 (m, 3H); MS: m/z 484 (M+1).

### Example 556

### 2,4-Difluoro-N-(4-(2-((trifluoromethylsulfonamido)methyl)thiazol-5-yl)phenyl)benzenesulfonamide

The compound of example 556 was prepared analogous to the compound of example 553 by reaction of the compound of example 542 with 2,4-difluorobenzenesulfonyl chloride. Yield: 60%; ¹H NMR (DMSO-d₆, 300 MHz): δ 10.90 (s, 1H), 10.48 (bs, 1H), 8.05 (s, 1H), 7.98-7.90 (m, 1H), 7.58-7.55 (d, 2H), 7.52-7.51 (m, 1H), 7.31-7.25 (m, 1H), 7.18-7.15 (d, 2H), 4.67 (s, 2H); MS: m/z 514 (M+1).

### Example 557

### t-Butyl (2-methyl-1-((2-(4-nitrophenyl)-2-oxoethyl)amino)-1-oxopropan-2-yl)carbamate

The compound of example 557 was prepared analogous to the compound of example 516 by reaction of the compound of example 2 with 2-(tert-butoxycarbonylamino)-2-methylpropanoic acid. Yield: 72%; ¹H NMR (DMSO-d₆, 300 MHz): δ 8.35-8.32 (d, 2H), 8.20-8.17 (m, 3H), 7.96-7.93 (t, 1H), 6.95 (bs, 1H), 4.58-4.56 (d, 2H), 3.63-3.61 (m, 2H), 1.36 (s, 9H), 1.30 (s, 6H); MS: m/z 364.2 (M−1).

### Example 558

### t-Butyl (2-(5-(4-nitrophenyl)thiazol-2-yl)propan-2-yl)carbamate

The compound of example 558 was prepared analogous to the compound of example 517 by reaction of the compound of example 557 with Lawesson's reagent. Yield: 61%; ¹H NMR (DMSO-d₆, 300 MHz): δ 8.28 (s, 1H), 8.27-8.25 (d, 2H), 7.92-7.89 (d, 2H), 7.72 (t, 1H), 1.60 (s, 6H), 1.36 (s, 9H); MS: m/z 364.1 (M+1).

### Example 559

### 2-(5-(4-Nitrophenyl)thiazol-2-yl)propan-2-amine hydrochloride

The compound of example 559 was prepared analogous to the compound of example 518 by reaction of the compound of example 558 with HCl. Yield: 77%;

¹H NMR (DMSO-d₆, 300 MHz): δ 8.30 (s, 1H), 8.27-8.24 (d, 2H), 7.93-7.90 (d, 2H), 2.44 (bs, 2H), 1.47 (s, 6H); MS: m/z 262.1 (M−1).

### Example 560

### 1,1,1-Trifluoro-N-(2-(5-(4-nitrophenyl)thiazol-2-yl)propan-2-yl)methane sulfonamide

The compound of example 560 was prepared analogous to the compound of example 519 by reaction of the compound of example 559 with triflic anhydride. Yield: 89%; ¹H NMR (DMSO-d₆, 300 MHz): δ 10.26 (s, 1H), 8.41 (s, 1H), 8.30-8.27 (d, 2H), 7.99-7.96 (d, 2H), 3.47 (s, 6H); MS: m/z 396 (M+1).

### Example 561

### N-(2-(5-(4-Aminophenyl)thiazol-2-yl)propan-2-yl)-1,1,1-trifluoromethane sulfonamide

The compound of example 561 was prepared analogous to the compound of example 378 by reduction of compound of example 560. Yield: 61%; ¹H NMR (DMSO-d₆, 300 MHz): δ 10.05 (bs, 1H), 7.80 (s, 1H), 7.31-7.29 (d, 2H), 6.61-6.58 (d, 2H), 5.49 (bs, 2H), 1.73 (s, 6H); MS: m/z 366 (M+1).

### Example 562

### N-(2-(5-(4-(3-(2-Chlorophenyl)ureido)phenyl)thiazol-2-yl)propan-2-yl)-1,1,1-trifluoromethanesulfonamide

The compound of example 562 was prepared analogous to the compound of example 6 by reaction of the compound of example 561 with 2-chloro-1-isocyanatobenzene.

Yield: 65%; ¹H NMR (DMSO-d₆, 300 MHz): δ 10.15 (s, 1H), 9.60 (s, 1H), 8.35 (s, 1H), 8.18-8.15 (dd, 1H), 8.03 (s, 1H), 7.63-7.53 (dd, 4H), 7.48-7.45 (dd, 1H), 7.34-7.29 (m, 1H), 7.07-7.02 (m, 1H), 1.76 (s, 6H); MS: m/z 519.1 (M+1).

### Example 563

### 1,1,1-Trifluoro-N-(2-(5-(4-(3-(2-fluorophenyl)ureido)phenyl)thiazol-2-yl) propan-2-yl)methanesulfonamide

The compound of example 563 was prepared analogous to the compound of example 6 by reaction of the compound of example 561 with 2-fluoro-1-isocyanatobenzene.

Yield: 75%; ¹H NMR (DMSO-d₆, 300 MHz): δ 10.14 (s, 1H), 9.26 (s, 1H), 8.59 (s, 1H), 8.18-8.12 (dd, 1H), 8.02 (s, 1H), 7.62-7.52 (dd, 4H), 7.28-7.22 (m, 1H), 7.18-7.13 (m, 1H), 7.06-7.01 (m, 1H), 1.75 (s, 6H); MS: m/z 503.1 (M+1).

### Example 564

### N-(2-(5-(4-(3-(3,5-Difluorophenyl)ureido)phenyl)thiazol-2-yl)propan-2-yl)-1,1,1-trifluoromethanesulfonamide

The compound of example 564 was prepared analogous to the compound of example 6 by reaction of the compound of example 561 with 3,5-difluoro-1-isocyanatobenzene.

Yield: 83%; ¹H NMR (DMSO-d₆, 300 MHz): δ 10.15 (s, 1H), 9.14 (s, 1H), 9.05 (s, 1H), 8.02 (s, 1H), 7.62-7.52 (dd, 4H), 7.21-7.18 (m, 2H), 6.84-6.81 (m, 1H), 1.75 (s, 6H); MS: m/z 521.1 (M+1).

### Example 565

### 1,1,1-Trifluoro-N-(2-(5-(4-(3-(2,4,5-trifluorophenyl)ureido)phenyl)thiazol-2-yl)propan-2-yl)methanesulfonamide

The compound of example 565 was prepared analogous to the compound of example by reaction of the compound of example 561 with 2,4,5-trifluoro-1-isocyanatobenzene. Yield: 75%; ¹H NMR (DMSO-d₆, 300 MHz): δ 10.15 (s, 1H), 9.27 (s, 1H), 8.76 (s, 1H), 8.22-8.18 (m, 1H), 8.03 (s, 1H), 7.67-7.59 (m, 3H), 7.54-7.51 (m, 2H), 1.75 (s, 6H); MS: m/z 539.1 (M+1).

### Example 566

### 1,1,1-Trifluoro-N-(2-(5-(4-(3-(2,4,6-trifluorophenyl)ureido)phenyl)thiazol-2-yl) propan-2-yl)methanesulfonamide

The compound of example 566 was prepared analogous to the compound of example by reaction of the compound of example 561 with 2,4,6-trifluoro-1-isocyanatobenzene. Yield: 72%; ¹H NMR (DMSO-d₆, 300 MHz): δ 10.14 (s, 1H), 9.19 (s, 1H), 8.08 (s, 1H), 8.01 (s, 1H), 7.60-7.51 (dd, 4H), 7.31-7.25 (m, 2H), 1.75 (s, 6H); MS: m/z 539.1 (M+1).

### Example 567

### N-(2-(5-(4-(3-Cyclohexylureido)phenyl)thiazol-2-yl)propan-2-yl)-1,1,1-trifluoromethanesulfonamide

The compound of example 567 was prepared analogous to the compound of example 6 by reaction of the compound of example 561 with cyclohexyl isocyanate. Yield: 69%;

¹H NMR (DMSO-d₆, 300 MHz): δ 10.13 (s, 1H), 8.50 (s, 1H), 7.97 (s, 1H), 7.53-7.50 (d, 2H), 7.46-7.43 (d, 2H), 6.14-6.11 (d, 1H), 3.46-3.42 (m, 1H), 1.82-1.60 (m, 10H), 1.59-1.49 (m, 1H), 1.36-1.15 (m, 5H); MS: m/z 491.1 (M+1).

### Example 568

### N-(4-(2-(2-(Trifluoromethylsulfonamido)propan-2-yl)thiazol-5-yl)phenyl)benzenesulfonamide

The compound of example 568 was prepared analogous to the compound of example 553 by reaction of the compound of example 561 with benzenesulfonyl chloride. Yield: 74%; ¹H NMR (DMSO-d₆, 300 MHz): δ 10.51 (s, 1H), 10.13 (s, 1H), 7.98 (s, 1H), 7.80-7.77 (d, 2H), 7.62-7.60 (m, 2H), 7.58-7.52 (m, 3H), 7.17-7.14 (d, 2H), 1.72 (s, 6H); MS: m/z 506.1 (M+1).

### Example 569

### t-Butyl (2-(5-(4-aminophenyl)thiazol-2-yl)ethyl)carbamate

The compound of example 569 was prepared analogous to the compound of example 378 by reduction of compound of example 517. Yield: 70%; ¹H NMR (DMSO-d₆, 300 MHz): δ 7.57 (s, 1H), 7.26-7.24 (d, 2H), 6.98 (t, 1H), 6.59-6.56 (d, 2H), 5.38 (bs, 2H), 3.32 (m, 2H), 3.02 (m, 2H), 1.37 (s, 9H); MS: m/z 320.1 (M+1).

### Example 570

### t-Butyl (2-(5-(4-(3-(2-chlorophenyl)ureido)phenyl)thiazol-2-yl)ethyl)carbamate

The compound of example 570 was prepared analogous to the compound of example 6 by reaction of the compound of example 569 with 2-chloro-1-isocyanatobenzene.

Yield: 80%; ¹H NMR (DMSO-d₆, 300 MHz): δ 9.58 (s, 1H), 8.35 (s, 1H), 8.18-8.15 (dd, 1H), 7.98 (s, 1H), 7.58-7.51 (dd, 4H), 7.34-7.28 (dd, 1H), 7.07-7.01 (m, 2H), 3.31-3.27 (m, 2H), 3.09-3.05 (m, 2H), 1.37 (s, 9H); MS: m/z 505 (M+1).

### Example 571

### t-Butyl (2-(5-(4-(3-(3,5-difluorophenyl)ureido)phenyl)thiazol-2-yl)ethyl)carbamate

The compound of example 571 was prepared analogous to the compound of example 6 by reaction of the compound of example 569 with 3,5-difluoro-1-isocyanatobenzene.

Yield: 81%; ¹H NMR (DMSO-d₆, 300 MHz): δ 9.13 (s, 1H), 9.03 (s, 1H), 7.98 (s, 1H), 7.57-7.50 (dd, 4H), 7.21-7.18 (m, 2H), 7.02 (t, 1H), 6.84-6.77 (m, 1H), 3.31-3.27 (m, 2H), 3.09-3.05 (m, 2H), 1.37 (s, 9H); MS: m/z 475.2 (M+1).

### Example 572

### t-Butyl (2-(5-(4-(3-(2,4,5-trifluorophenyl)ureido)phenyl)thiazol-2-yl)ethyl)carbamate

The compound of example 572 was prepared analogous to the compound of example 6 by reaction of the compound of example 569 with 2,4,5-trifluoro-1-isocyanatobenzene. Yield: 91%; ¹H NMR (DMSO-d₆, 300 MHz): δ 9.24 (s, 1H), 8.76 (s, 1H), 8.24-8.14 (m, 1H), 7.98 (s, 1H), 7.69-7.63 (m, 1H), 7.62-7.49 (dd, 4H), 7.01 (t, 1H), 6.84-6.77 (m, 1H), 3.29-3.25 (m, 2H), 3.09-3.05 (m, 2H), 1.37 (s, 9H); MS: m/z 493.2 (M+1).

### Example 573

### 1-(4-(2-(2-Aminoethyl)thiazol-5-yl)phenyl)-3-(2-chlorophenyl)urea hydrochloride

The compound of example 573 was prepared analogous to the compound of example 518 by reaction of the compound of example 570 with HCl. Yield: 95%;

¹H NMR (DMSO-d₆, 300 MHz): δ 9.24 (s, 1H), 8.55 (s, 1H), 8.14-8.12 (m, 1H), 8.04 (s, 1H), 7.57 (dd, 4H), 7.46-7.43 (m, 1H), 7.32-7.27 (m, 1H),7.07-7.00 (m, 1H), 4.60 (bs, 2H), 3.34-3.30 (m, 2H), 3.26-3.22 (m, 2H); MS: m/z 373.1 (M+1).

### Example 574

### 1-(4-(2-(2-Aminoethyl)thiazol-5-yl)phenyl)-3-(3,5-difluorophenyl)urea hydrochloride

The compound of example 574 was prepared analogous to the compound of example 518 by reaction of the compound of example 571 with HCl. Yield: 89%; ¹H NMR (DMSO-d₆, 300 MHz): δ 9.93 (s, 1H), 9.63 (s, 1H), 8.09 (bs, 1H), 8.04 (s, 1H), 7.59-7.50 (dd, 4H), 7.18-7.15 (m, 2H), 6.81-6.74 (m, 1H), 4.44 (bs, 2H), 3.30-3.26 (m, 2H), 3.25-3.22 (m, 2H); MS: m/z 375.1 (M+1).

### Example 575

### 1-(4-(2-(2-Aminoethyl)thiazol-5-yl)phenyl)-3-(2,4,5-trifluorophenyl)urea hydrochloride

The compound of example 575 was prepared analogous to the compound of example 518 by reaction of the compound of example 572 with HCl. Yield: 72%;

¹H NMR (DMSO-d₆, 300 MHz): δ 9.90 (s, 1H), 9.07 (s, 1H), 8.23-8.19 (m, 1H), 8.18-8.11 (bs, 1H), 8.05 (s, 1H), 7.68-7.64 (m, 1H), 7.62-7.52 (dd, 4H), 4.40 (bs, 2H), 3.34-3.30 (m, 2H), 3.26-3.23 (m, 2H); MS: m/z 393.1 (M+1).

### Example 576

### 2,2-Dimethyl-4-(5-(4-nitrophenyl)thiazol-2-yl)butanoic acid

To a solution of the compound of example 85 (11 g, 32.9 mmol) in methanol (110 mL) and THF (110 mL) was added 1N NaOH solution (164 mL, 164 mmol) and stirred at room temperature for 24 h. The organic solvent was removed and the reaction mixture was poured into water, acidified to pH 2-3 with dilute aqueous hydrochloric acid, and extracted with ethyl acetate. The combined organic layers were dried over sodium sulfate and evaporated to dryness to obtain a solid, which was crystallized in ethyl acetate-petroleum ether to afford the title compound. Yield: 9.6 g (91%); ¹H NMR (DMSO-d₆, 300 MHz): δ 12.31 (bs, 1H), 8.34 (s, 1H), 8.28-8.25 (d, 2H), 7.93-7.90 (d, 2H), 2.99 (m, 2H), 1.96 (m, 2H), 1.18 (s, 6H); MS: m/z 321.1 (M+1).

### Example 577

### 2,2-Dimethyl-4-(5-(4-nitrophenyl)thiazol-2-yl)-N-((trifluoromethyl)sulfonyl)butanamide

The compound of example 576 (500 mg, 1.561 mmol) was dissolved in THF (15 mL) to which N-methylmorpholine (0.172 mL, 1.561 mmol) was added and the mixture was cooled to −20° C. to −30° C. To this reaction mixture, isobutyl chloroformate (0.205 mL, 1.561 mmol) was added and stirred for an additional 30 min at the same temperature. Trifluoromethanesulfonamide (256 mg, 1.717 mmol) in THF (5 mL) and 2,3,4,6,7,8,9,10-octahydropyrimido[1,2-a]azepine (261 mg, 1.717 mmol) were added to the above reaction mixture and stirred at −20° C. to −30° C. for 10 min and the reaction mixture was warmed to room temperature gradually over an hour. The reaction mixture was refluxed for 16 h. The reaction was quenched by addition of water and extracted with ethyl acetate. The organic layer was dried over sodium sulfate and evaporated under vacuum to obtain a residue, which was purified by flash column chromatography (silica gel, 30% acetone in chloroform) to afford the title compound. Yield: 352 mg (50%); ¹H NMR (DMSO-d₆, 300 MHz): δ 8.31 (s, 1H), 8.27-8.24 (d, 2H), 7.93-7.90 (d, 2H), 2.92 (m, 2H), 1.88 (m, 2H), 1.06 (s, 6H); MS: m/z 452 (M+1).

### Example 578

### 4-(5-(4-Aminophenyl)thiazol-2-yl)-2,2-dimethyl-N-((trifluoromethyl)sulfonyl)butanamide

The compound of example 578 was prepared analogous to the compound of example 378 by reduction of compound of example 577. Yield: 62%; ¹H NMR (DMSO-d₆, 300 MHz): δ 7.69 (s, 1H), 7.26-7.23 (d, 2H), 6.58-6.55 (d, 2H), 5.35 (bs, 2H), 2.83 (m, 2H), 1.85 (m, 2H), 1.06 (s, 6H); MS: m/z 422 (M+1).

### Example 579

### 4-(5-(4-(3-(2-Chlorophenyl)ureido)phenyl)thiazol-2-yl)-2,2-dimethyl-N-((trifluoromethyl)sulfonyl)butanamide

The compound of example 579 was prepared analogous to the compound of example 6 by reaction of the compound of example 578 with 2-chloro-1-isocyanatobenzene.

Yield: 48%; ¹H NMR (DMSO-d₆, 300 MHz): δ 9.56 (s, 1H), 8.34 (s, 1H), 8.18-8.15 (d, 1H), 7.92 (s, 1H), 7.67-7.49 (dd, 4H), 7.47-7.44 (m, 1H), 7.33-7.28 (m, 1H), 7.05-7.00 (m, 1H), 2.85 (m, 2H), 1.85 (m, 2H), 1.18 (s, 6H); MS: m/z 575.1 (M+1).

### Example 580

### 4-(5-(4-(3-(2-Fluorophenyl)ureido)phenyl)thiazol-2-yl)-2,2-dimethyl-N-((trifluoromethyl)sulfonyl)butanamide

The compound of example 580 was prepared analogous to the compound of example 6 by reaction of the compound of example 578 with 2-fluoro-1-isocyanatobenzene.

Yield: 57%; ¹H NMR (DMSO-d₆, 300 MHz): δ 9.22 (s, 1H), 8.58 (s, 1H), 8.18-8.12 (m, 1H), 7.92 (s, 1H), 7.56-7.48 (dd, 4H), 7.27-7.21 (m, 1H), 7.17-7.12 (m, 1H), 7.05-6.98 (m, 1H), 2.86 (m, 2H), 1.86 (m, 2H), 1.06 (s, 6H); MS: m/z 559.1 (M+1).

### Example 581

### 4-(5-(4-(3-(3,5-Difluorophenyl)ureido)phenyl)thiazol-2-yl)-2,2-dimethyl-N-((trifluoromethyl)sulfonyl)butanamide

The compound of example 581 was prepared analogous to the compound of example 6 by reaction of the compound of example 578 with 3,5-difluoro-1-isocyanatobenzene. Yield: 64%; ¹H NMR (DMSO-d₆, 300 MHz): δ 9.13 (s, 1H), 9.02 (s, 1H), 7.92 (s, 1H), 7.56-7.49 (dd, 4H), 7.21-7.18 (m, 2H), 6.83-6.71 (m, 1H), 2.86 (m, 2H), 1.85 (m, 2H), 1.06 (s, 6H); MS: m/z 577.1 (M+1).

### Example 582

### 2,2-Dimethyl-N-((trifluoromethyl)sulfonyl)-4-(5-(4-(3-(2,4,5-trifluorophenyl)ureido)phenyl)thiazol-2-yl)butanamide

The compound of example 582 was prepared analogous to the compound of example 6 by reaction of the compound of example 578 with 2,4,5-trifluoro-1-isocyanatobenzene. Yield: 44%; ¹H NMR (DMSO-d₆, 300 MHz): δ 9.24 (s, 1H), 8.76 (s, 1H), 8.24-8.15 (m, 1H), 7.92 (s, 1H), 7.68-7.62 (m, 1H), 7.56-7.48 (dd, 4H), 2.86 (m, 2H), 1.86 (m, 2H), 1.06 (s, 6H); MS: m/z 595.1 (M+1).

### Example 583

### Methyl 4-(5-(4-(3-(2,4,5-trifluorophenyl)ureido)phenyl)thiazol-2-yl)cyclohexane carboxylate

The compound of example 583 was prepared analogous to the compound of example by reaction of the compound of example 132 with 2,4,5-trifluoro-1-isocyanatobenzene. Yield: 97%; ¹H NMR (DMSO-d₆, 300 MHz): δ 9.23 (s, 1H), 8.75 (s, 1H), 8.24 (m, 1H), 7.96 (s, 1H), 7.69 (m, 1H), 7.57 (d, 2H), 7.51 (d, 2H), 3.61 (m, 3H), 2.97 (m, 1H), 2.41 (m, 1H), 2.12 (m, 2H), 2.02 (m, 2H), 1.57 (m, 4H); MS: m/z 490.1 (M+1).

### Example 584

### 4-(5-(4-(3-(2,4,5-Trifluorophenyl)ureido)phenyl)thiazol-2-yl)cyclohexane carboxylic acid

The compound of example 584 was prepared analogous to the compound of example 7 by hydrolysis of the compound of example 583. Yield: 85%; ¹H NMR (DMSO-d₆, 300 MHz): δ 9.52 (s, 1H), 8.89 (s, 1H), 8.21 (m, 1H), 7.98 (s, 1H), 7.68 (m, 1H), 7.58 (d, 2H), 7.52 (d, 2H), 2.96 (m, 1H), 2.27 (m, 1H), 2.15 (m, 2H), 2.02 (m, 2H), 1.57 (m, 4H); MS: m/z 476 (M+1).

### Example 585

### 1-(4-(2-(4-(2-Hydroxypropan-2-yl)cyclohexyl)thiazol-5-yl)phenyl)-3-(2,4,5-trifluorophenyl)urea

The compound of example 585 was prepared analogous to the compound of example 404 by reaction of compound of example 583 with methyl magnesium bromide. Yield: 34%; ¹H NMR (DMSO-d₆, 300 MHz): δ 9.21 (s, 1H), 8.73 (5, 1H), 8.22 (m, 1H), 7.93 (s, 1H), 7.67 (m, 1H), 7.55 (d, 2H), 7.49 (d, 2H), 4.07 (5, 1H), 2.89 (m, 1H), 2.16 (m, 2H), 1.91 (m, 2H), 1.49 (m, 2H), 1.25 (m, 3H), 1.04 (5, 6H); MS: m/z 490.2 (M+1).

### Example 586

### 2-Chloro-N-(2-(4-(5-(4-(3-(2,4,5-trifluorophenyl)ureido)phenyl)thiazol-2-yl)cyclohexyl)propan-2-yl)acetamide

To a solution of the compound of example 585 (125 mg, 0.255 mmol) in acetic acid (2 mL) was added 2-chloroacetonitrile (38.6 mg, 0.511 mmol) and this reaction mixture was cooled to 0-5° C. Sulfuric acid (0.027 mL, 0.511 mmol) was slowly added while keeping the temperature of this reaction mixture below 10° C. Following the addition of sulfuric acid, the reaction mixture was stirred at room temperature for 16 h. After completion of the reaction, water was added and the precipitated solid was extracted using ethyl acetate. The organic layer was washed with a saturated solution of sodium bicarbonate, concentrated and the resulting solid was stirred in dichloromethane and petroleum ether, filtered, and dried to afford the title compound. Yield: 125 mg (86%);

¹H NMR (DMSO-d₆, 300 MHz): δ 9.24 (s, 1H), 8.76 (s, 1H), 8.25 (m, 1H), 7.96 (s, 1H), 7.69 (m, 2H), 7.57 (d, 2H), 7.52 (d, 2H), 4.00 (s, 2H), 2.94 (m, 1H), 2.19 (m, 2H), 2.02 (m, 1H), 1.82 (m, 2H), 1.51 (m, 2H), 1.22 (m, 2H), 1.18 (s, 6H); MS: m/z 565.2 (M+1).

### Example 587

### 1-(4-(2-(4-(2-Aminopropan-2-yl)cyclohexyl)thiazol-5-yl)phenyl)-3-(2,4,5-trifluoro phenyl)urea

A solution of the compound of example 586 (125 mg, 0.221 mmol) and thiourea (25.3 mg, 0.332 mmol) in ethanol (5 mL) and acetic acid (0.5 mL) was stirred for 3 h at 85° C. After completion of the reaction, dilute NaOH solution was added to maintain the pH neutral followed by addition of water. The resulting solution was extracted using ethyl acetate. The organic layer was washed with water and brine, dried over sodium sulfate and concentrated to afford the title compound. Yield: 85 mg (76%); ¹H NMR (DMSO-d₆, 300 MHz): δ 9.31 (s, 1H), 8.83 (s, 1H), 8.23 (m, 1H), 7.94 (s, 1H), 7.68 (m, 1H), 7.57 (d, 2H), 7.52 (d, 2H), 2.91 (m, 1H), 2.19 (m, 2H), 1.91 (m, 1H), 1.52 (m, 2H), 1.19 (m, 2H), 0.98 (m, 8H); MS: m/z 489.2 (M+1).

### Example 588

### 2-Chloro-N-(2-(4-(5-(4-(3-(2,4-difluorophenyl)ureido)phenyl)thiazol-2-yl)cyclohexyl)propan-2-yl)acetamide

The compound of example 588 was prepared analogous to the compound of example 586 by reaction of compound of example 406 with 2-chloroacetonitrile. Yield: 62%; ¹H NMR (DMSO-d₆, 300 MHz): δ 9.32 (s, 1H), 8.69 (s, 1H), 8.09 (m, 1H), 7.94 (s, 1H), 7.66 (s, 1H), 7.56 (m, 4H), 7.35 (m, 1H), 7.06 (m, 1H), 3.99 (s, 2H), 2.90 (m, 1H), 2.18 (m, 2H), 1.99 (m, 1H), 1.81 (m, 2H), 1.50 (m, 2H), 1.25 (m, 2H), 1.22 (s, 6H); MS: m/z 547.2 (M+1).

### Example 589

### 1-(4-(2-(4-(2-Aminopropan-2-yl)cyclohexyl)thiazol-5-yl)phenyl)-3-(2,4-difluoro phenyl)urea

The compound of example 589 was prepared analogous to the compound of example 587 by reaction of compound of example 588 with thiourea and acetic acid. Yield: 65%; ¹H NMR (DMSO-d₆, 300 MHz): δ 9.41 (s, 1H), 8.73 (s, 1H), 8.09 (m, 1H), 7.94 (s, 1H), 7.52 (m, 4H), 7.34 (m, 1H), 7.07 (m, 1H), 2.89 (m, 1H), 2.19 (m, 2H), 1.91 (m, 2H), 1.48 (m, 2H), 1.24 (m, 3H), 1.05 (s, 6H); MS: m/z 471.2 (M+1).

### Example 590

### 2-Chloro-N-(2-methyl-1-(4-(5-(4-(3-(2,4,5-trifluorophenyl)ureido)phenyl)thiazol-2-yl)cyclohexyl)propan-2-yl)acetamide

The compound of example 590 was prepared analogous to the compound of example 586 by reaction of compound of example 409 with 2-chloroacetonitrile. Yield: 69%; ¹H NMR (DMSO-d₆, 300 MHz): δ 9.26 (s, 1H), 8.76 (s, 1H), 8.21 (m, 1H), 7.94 (s, 1H), 7.72 (s, 1H), 7.69 (m, 1H), 7.57 (d, 2H), 7.51 (d, 2H), 3.96 (s, 2H), 2.94 (m, 1H), 2.08 (m, 2H), 1.91 (m, 2H), 1.63 (d, 2H), 1.54 (m, 2H), 1.40 (m, 1H), 1.25 (s, 6H), 1.17 (m, 2H); MS: m/z 579.2 (M+1).

### Example 591

### 1-(4-(2-(4-(2-Amino-2-methylpropyl)cyclohexyl)thiazol-5-yl)phenyl)-3-(2,4,5-trifluorophenyl)urea

The compound of example 591 was prepared analogous to the compound of example 587 by reaction of compound of example 590 with thiourea and acetic acid. Yield: 57%; ¹H NMR (DMSO-d₆, 300 MHz): δ 9.30 (s, 1H), 8.83 (s, 1H), 8.23 (m, 1H), 7.94 (s, 1H), 7.68 (s, 1H), 7.56 (d, 2H), 7.51 (d, 2H), 2.92 (m, 1H), 2.08 (m, 2H), 1.90 (m, 2H), 1.57 (d, 2H), 1.46 (m, 1H), 1.23 (d, 2H), 1.17 (m, 2H), 1.03 (s, 6H); MS: m/z 503.2 (M+1).

### Pharmacology Data

The efficacy of the compounds of the present invention can be determined by a number of pharmacological assays well known in the art, such as described below. The exemplified pharmacological assays, which follow herein below, have been carried out with the compounds of the present invention.

### Materials:

Bovine serum albumin (BSA), (Sigma)

### Bradford (Sigma)

### ¹⁴C Oleoyl CoA (GE Healthcare)

### Cellfectin (Invitrogen)

Fetal bovine serum (FBS), (Hyclone)

### Heptane (Qualigens)

2-propanol (Qualigens)
Sf9 cells were obtained from American Type Culture Collection (ATCC)
sn-1,2-dioleoylglycerol (Sigma)

### Sucrose (Sigma)

Tissue culture materials, (Nunc)
Tissue culture media, (Gibco)

### Abbreviations or Terms Used:

AESSM: Alkaline Ethanol Stop Solution Mix
ALT: Alanine aminotransferase
AST: Aspartate aminotransferase
BSA: Bovine serum albumin
CMC: Carboxy methyl cellulose
DAB: DGAT Assay Buffer
DNA: Deoxyribonucleic acid
EDTA: Ethylene Diamine Tetraacetic Acid
FBS: Fetal Bovine serum
HFD: High fat diet
hDGAT1: Human diacylglycerol acyltransferase
hDGAT10RF: Human diacylglycerol acyltransferase Open Reading frame
IC₅₀: Half maximal inhibitory concentration
IVC: Individually ventilated cages
IU/L: International units per liter
KCl: Potassium chloride
KH₂PO₄: Potassium Dihydrogen Phosphate
Kcal/g: Kilocalory per gram
LB: Luria Bertani
LFD: Low fat diet
mL/kg: Milliliter per kilogram
μg/mL: Microgram per milliliter
μg: Microgram
μm: Micrometer
Mm: Millimolar
nM: Nanomolar
NPD: Normal pellet diet
ORF: Open Reading Frame
p.o: oral administration
p.o., b.i.d: Oral administration twice a day
POPOP: 1,4-bis(5-phenyloxazol-2-yl)benzene
PPO: 2,5-Diphenyloxazole
S.E.M: Standard error of the mean
units/mL: Units per milliliter

### Example 592

### In-vitro Protocol for DGAT1 assay

### Sf9 Culture and Treatment

Sf9 cells were grown in T25 flasks containing Graces's Insect media with 10% FBS with antibiotic (100 units/mL penicillin, 100 μg/mL streptomycin sulphate, 0.25 μg/mL Amptotericin B as Fungizone) grown in a 27° C. incubator.

### Viral Stock Preparation

hDGAT10RF expression clone (RZPDo839C09146 in pDEST vector) was obtained from RZPD, German Science Center for Genomes research, Germany. hDGAT1 bacmid DNA was obtained by transformation of the hDGAT1 expression clone into DH10Bac E. coli competent cells. Approximately 1 μg of hDGAT1 bacmid DNA was infected into Sf9 cells with Cellfectin (Invitrogen) reagent. Following infection, Sf9 cells were incubated at 27° C. for 30 min. Five hours after infection, the media was replaced with growth media containing antibiotics (100 units/mL penicillin, 100 μg/mL streptomycin sulphate, 0.25 μg/mL Amptotericin B as Fungizone) and incubated at 27° C. for 72 h. The supernatant containing the virus was centrifuged at 1500×g for 5 min, passed through 0.22 μm filter, and subsequently stored at 4° C. The virus was further amplified three more times by re-infection of Sf9 cells and the viral titer was determined by plaque assay.

Preparation of hDGAT1 Microsomes from Sf9 Cells

Sf9 cells were seeded in spinner flasks on day 0 at a cell density of 1×10⁶ and infected on day 1 with hDGAT1 baculovirus at a multiplicity of infection (MOI) of 5 and a cell density of 2×10⁶. On day 3 (or 66-72 h), cells were harvested and centrifuged at 2500×g for 10 min. Pellet was resuspended in lysis buffer (100 mM sucrose, 50 mM KCl, 40 mM KH₂PO₄, 30 mM EDTA, pH 7.2) and passed through 21-gauge needle approximately 10 times. The mixture was centrifuged at 12,000 rpm in a Sigma 12158-H rotor at 4° C. for 30 min. The supernatant was subjected to centrifugation at 35,000 rpm in a Beckman Ti-45 rotor at 4° C. for 1 h. The resultant pellet containing the microsomes wasere resuspended overnight in 1 mL of lysis buffer and total protein concentration was estimated using Bradford Reagent. Microsomes were aliquoted and stored at −80° C.

### Measurement of DGAT1 Activity

Frozen aliquots of hDGAT1 containing microsomes were thawed (5-10 mg/mL total protein) on ice and diluted to a working stock of 1 mg/mL with DGAT Assay Buffer (DAB). The DGAT reaction assay was performed by following the procedure described in U.S. Pat. No. 6,607,893 with some modifications that are described below.

### Preparation of DGAT1 Substrate Mixture:

1 mL stock solution of DGAT1 substrate mixture contains 5.6 μL of ¹⁴C oleoyl CoA (16.8 nCi) and 105 μL of 1,2-dioleoyl-sn-glycerol (1228.5 μM) 1,2-dioleoyl-sn-glycerol stock (19.5 mM) was prepared by dissolving 25 mg of 1,2-dioleoyl-sn-glycerol (Sigma, US) in 2060 μL of acetone.

The assay was performed in duplicates in a reaction volume of 100 μL. The reaction volume consisted of:
(i) 27.5 μL of DGAT assay buffer (0.25 M Sucrose, 1 mM EDTA (pH 8.0), 150 mM Tris-HCl, pH 7.4, 1.25 mg/mL fatty acid free BSA),
(ii) 10 μL of compound of present invention or standard (2-(4′-(6-fluorobenzo[d]thiazol-2-ylamino)biphenyl-4-ylcarboxamido)-3-methylbutanoic acid) (dissolved in DMSO and diluted to 10× with DAB and screened at 10 μM, 5 μM and 1 μM),
(iii) 60 μL DGAT1 substrate mixture taken from a 1 mL stock (16.8 nCi of ¹⁴C oleoyl CoA and 1228.5 μM of 1,2-dioleyl-sn-glycerol),
(iv) 2.5 μL of 1 mg/mL of microsomes (the amount of assay buffer was varied depending upon the concentration of microsome to make up the volume to 100 μL).

### Procedure:

The reaction was started by the addition of 2.5 μL of 1 mg/mL of microsomes (iv) to the mixture of (i), (ii) and (iii), and incubated at 37° C. for 10 min. The reaction was stopped by the addition of 300 μL of Alkaline Ethanol Stop Solution Mix [AESSM; 12.5% of 100% non-denatured ethanol, 10% deionized water, 2.5% 1N NaOH, 75% stop solution (78.4% isopropanol, 19.6% n-heptane, 2% deionized water)] followed by addition of 600 μL of n-heptane. The mixture was vortexed and the triglycerides formed were extracted into the organic heptane phase. 250 μL of the heptane phase was added into 4 mL scintillation cocktail (66.72% toluene, 33.3% TritonX-100, 0.5% PPO, 0.02% POPOP) and counted on a liquid scintillation counter for 1 min. Data was collected and plotted as a function of concentration in nM versus percentage inhibition of hDGAT1 by the compounds of present invention. Inhibitor concentration at 50% (IC₅₀) was determined using 8-point concentration values (0.1 nM, 0.3 nM, 10 nM, 30 nM, 100 nM, 300 nM, 1000 nM and 3000 nM). The IC₅₀ values of representative examples of the present invention were found to be in the range of 1-1000 nM. The % inhibition of hDGAT1 at 1 μM is displayed in Table 1 for representative examples of the present invention.

TABLE 1
% Inhibition of hDGAT1
(Scoring Details)

| Example | Percentage |
|---|---|
| No. of | inhibition of |
| compound | hDGAT1 |
| 7 | ++ |
| 9 | ++ |
| 11 | ++ |
| 13 | ++ |
| 15 | ++ |
| 17 | ++ |
| 19 | ++ |
| 21 | ++ |
| 23 | ++ |
| 25 | + |
| 30 | ++ |
| 32 | ++ |
| 34 | + |
| 36 | ++ |
| 38 | + |
| 40 | ++ |
| 42 | + |
| 44 | + |
| 49 | ++ |
| 51 | ++ |
| 53 | ++ |
| 55 | ++ |
| 57 | + |
| 59 | ++ |
| 61 | ++ |
| 63 | + |
| 69 | ++ |
| 71 | ++ |
| 73 | ++ |
| 75 | ++ |
| 77 | ++ |
| 79 | ++ |
| 81 | + |
| 88 | ++ |
| 90 | ++ |
| 92 | ++ |
| 94 | ++ |
| 96 | ++ |
| 98 | ++ |
| 106 | ++ |
| 108 | ++ |
| 124 | ++ |
| 126 | + |
| 128 | + |
| 134 | ++ |
| 136 | ++ |
| 138 | ++ |
| 140 | ++ |
| 142 | ++ |
| 144 | ++ |
| 146 | ++ |
| 148 | ++ |
| 150 | ++ |
| 152 | ++ |
| 154 | ++ |
| 156 | ++ |
| 158 | ++ |
| 160 | ++ |
| 162 | ++ |
| 164 | ++ |
| 166 | ++ |
| 168 | ++ |
| 170 | ++ |
| 172 | ++ |
| 174 | ++ |
| 176 | + |
| 178 | ++ |
| 180 | ++ |
| 182 | ++ |
| 184 | ++ |
| 186 | ++ |
| 188 | ++ |
| 190 | ++ |
| 192 | ++ |
| 194 | ++ |
| 196 | + |
| 198 | ++ |
| 204 | + |
| 206 | + |
| 208 | ++ |
| 210 | + |
| 214 | + |
| 219 | + |
| 221 | + |
| 223 | + |
| 225 | ++ |
| 227 | ++ |
| 229 | + |
| 231 | + |
| 233 | + |
| 235 | ++ |
| 235A | ++ |
| 237 | ++ |
| 237A | ++ |
| 239 | + |
| 241 | + |
| 243 | + |
| 245 | ++ |
| 247 | + |
| 249 | + |
| 251 | ++ |
| 255 | ++ |
| 255A | ++ |
| 257 | + |
| 259 | ++ |
| 261 | + |
| 263 | ++ |
| 265 | + |
| 271 | + |
| 278 | ++ |
| 280 | ++ |
| 282 | ++ |
| 284 | ++ |
| 286 | ++ |
| 288 | + |
| 290 | + |
| 292 | + |
| 296 | + |
| 300 | + |
| 302 | + |
| 315 | + |
| 322 | ++ |
| 332 | + |
| 346 | ++ |
| 348 | ++ |
| 356 | ++ |
| 358 | ++ |
| 360 | ++ |
| 362 | ++ |
| 364 | ++ |
| 366 | ++ |
| 368 | ++ |
| 370 | ++ |
| 372 | ++ |
| 374 | ++ |
| 379 | ++ |
| 380 | + |
| 381 | ++ |
| 382 | ++ |
| 383 | ++ |
| 385 | + |
| 390 | ++ |
| 391 | ++ |
| 392 | ++ |
| 393 | ++ |
| 394 | ++ |
| 396 | + |
| 400 | ++ |
| 401 | ++ |
| 402 | ++ |
| 403 | + |
| 404 | ++ |
| 405 | ++ |
| 406 | ++ |
| 407 | ++ |
| 408 | ++ |
| 409 | ++ |
| 410 | ++ |
| 413 | ++ |
| 414 | ++ |
| 429 | ++ |
| 431 | ++ |
| 433 | ++ |
| 435 | ++ |
| 437 | ++ |
| 439 | + |
| 451 | ++ |
| 453 | ++ |
| 455 | ++ |
| 459 | ++ |
| 474 | + |
| 475 | + |
| 476 | + |
| 477 | + |
| 478 | + |
| 479 | + |
| 480 | + |
| 483 | + |
| 484 | + |
| 485 | + |
| 486 | + |
| 487 | + |
| 490 | + |
| 491 | + |
| 493 | + |
| 494 | ++ |
| 502 | ++ |
| 506 | ++ |
| 508 | ++ |
| 510 | ++ |
| 512 | ++ |
| 514 | ++ |
| 526 | ++ |
| 527 | ++ |
| 528 | ++ |
| 529 | + |
| 533 | ++ |
| 537 | ++ |
| 543 | ++ |
| 544 | ++ |
| 545 | ++ |
| 546 | ++ |
| 547 | ++ |
| 548 | ++ |
| 549 | ++ |
| 550 | ++ |
| 551 | + |
| 552 | ++ |
| 553 | + |
| 562 | ++ |
| 563 | ++ |
| 564 | ++ |
| 565 | ++ |
| 566 | + |
| 567 | ++ |
| 568 | + |
| 574 | + |
| 575 | + |
| 579 | ++ |
| 580 | ++ |
| 581 | ++ |
| 582 | ++ |
| 584 | ++ |
| 585 | ++ |
| 587 | ++ |
| 589 | ++ |
| 591 | ++ |

+ 20-50% Inhibition;
++ >50 % Inhibition

### Example 593

### In-Vivo Screening

Animals were housed and cared for in accordance with the Guidelines in force published by CPCSEA (Committee for the Purpose of Control and Supervision of Experiments on Animals), Tamil Nadu, India. Procedures using laboratory animals were approved by the IAEC (Institutional Animal Ethics Committee) of the Research Centre of Piramal Life Sciences Limited, Mumbai, India.

Study Protocol for Screening of Compounds for Fat Tolerance Test (ftt) in Mice

Swiss mice of age 4-5 weeks and body weight between 25-30 g were selected for study. After fasting for about 16 h, the animals were divided into three groups based on plasma triglyceride level with same mean and variation. Animals were administered with either vehicle [(1% tween 80 in 0.5% carboxy methylcellulose (CMC)]) or with representative compounds of the present invention (3 mg/kg, p.o.). Compounds of the present invention were prepared as suspension in 0.5% CMC with 1% tween 80. Olive oil (fat) load (10 mL/kg, p.o.) was given, 30 min after the treatment. Blood samples were collected at 1, 2, 3 and 4 h after the fat (olive oil) load. Plasma was separated and triglyceride level was measured using commercially available kits (diasys, Germany). Percentage reduction in area under curve (AUC ₀₋₄ₕ) of the test compound was calculated by taking AUC₀₋₄ₕ of the vehicle group as 100%. Certain examples of the present invention were screened for determining reduction in levels of plasma triglyceride. The examples screened showed more than 50% reduction in levels of plasma triglyceride.

References for In-Vivo Screening of Compounds:
1. Koji Ueshima, Hitomi Akihisa-Umeno, Akira Nagayoshi, Shoji Takakura, Masahiko Matsuo, Seitaro Mutoh. A gastrointestinal lipase inhibitor reduces progression of atherosclerosis in mice fed a western-type diet. European Journal of Pharmacology (2004), 501, 137-142.
2. L-K Han et al. “Anti-obesity effects in rodents of dietary teasaponin, a lipase inhibitor” International Journal of Obesity (2001), 25, 1459-1464.
3. Katherine J. D. Ashbourne Excoffon et al. “Correction of Hypertriglyceridemia and Impaired Fat Tolerance in Lipoprotein Lipase-Deficient Mice by Adenovirus-Mediated Expression of Human Lipoprotein Lipase” Arteriosclerosis, Thrombosis, and Vascular Biology (1997), 17, 2532-2539.

Additionally, one or more compounds of the present invention may be tested in any of the below-mentioned assays to determine their efficacy in obtaining a reduction in body weight, cumulative feed intake and/or biochemical parameters such as plasma glucose (mg/dL), plasma triglyceride (mg/dL), plasma cholesterol (mg/dL), plasma AST (IU/L), plasma ALT (IU/L) and liver weight (g).

### Example 594

### Chronic Study 1

### Effect of the Test Compound on High Fat Diet Induced Weight Gain in Ob/Ob Mice

### Meal-Fed Protocol

Male ob/ob mice aged 4-5 weeks with body weight range of 30-40 g are procured from the Jackson Laboratory, USA and kept in the central animal facility, Piramal Life Sciences Limited, Mumbai, India. Animals are housed in individually ventilated cages (IVC's) at a room temperature of 22±2° C., humidity 55±5% with a 12:12 h light-dark cycle and have access to water ad libitum. Mice (one/cage) are allowed to acclimatize on standard diet (normal pellet diet, NPD; Amrut Laboratory Animal Feed, India) for one week. Then animals are grouped based on body weight and plasma glucose with similar mean±S.E.M. with 10 animals per group.

### Acclimatization Period

All the mice are housed individually in IVC's cages and subjected to 9 days acclimatization period. In brief, animals are provided with either low fat diet (LFD) or high fat diet (HFD). LFD provides 10% of the total calories obtained from lard (D12450B; Research Diets Inc., NJ, USA) with total energy provided as 3.85 Kcal/g of feed whereas HFD provides 60% of the total calories obtained from lard (D12492; Research Diets Inc., NJ, USA) with total energy provided as 5.24 Kcal/g of feed. Animals are provided with ad libitum feed from day 1 to day 3. From day 4 to day 6, food is restricted for 12 h. From day 7 to day 9, food is provided for three h in the morning and three h in the evening. During acclimatization period, mice are administered with vehicle (1% Tween 80 in 0.5% CMC; 10 mL/kg) twice daily, to acclimatize them to oral dosing and handling procedures.

### Treatment Regimen

On day 10, high fat fed animals are regrouped to three groups based on body weight with similar mean±S.E.M. with 10 animals per group. The test compound is prepared as suspension with 1% Tween 80 in 0.5% CMC. Vehicle (0.5% CMC with 1% Tween 80; 10 ml/kg) or the test compound is administered twice daily in the morning and evening. The concentration of test compounds used is in the range of 0.1 to 1 mg/kg (p.o., b.i.d.). This dosing regimen is continued for 14 days. Daily body weight is recorded just before administration of test compound.

### Food Intake Measurement

Food intake is measured twice daily. In the morning, random amount of LFD or HFD is kept in the metallic lid. It is weighed with food and is considered as food provided. At noon, lid weight with food is measured as food remaining. Food intake in morning is calculated as difference between food provided and food remaining. Mice are devoid of food for six hours. In the evening, again food is provided and food intake is measured at 9 pm as per the above procedure during morning session. Followed by this, food is removed from the cages for 12 h. Sum of the food intake in the morning and in the evening gives total food intake during the corresponding day.

### Biochemical Parameters Estimation and Necropsy

Blood (˜80 μL) is collected from the retro-orbital plexus of mice on day 15, 1 h after administration of the test compound. The plasma is separated by centrifugation at 8000×g for 7 min at 4° C. and plasma glucose, triglyceride, cholesterol, liver enzymes [alanine aminotransferase (ALT) and aspartate aminotransferase (AST)], LDL-C and HDL-C are estimated immediately using a biochemistry autoanalyser (Hitachi Science Systems Limited, Ibaraki, Japan). Plasma insulin (Linco Research, USA) is estimated as per manufacturer's protocol.

Observations are recorded for percent change in body weight gain and cumulative feed intake during 14 days of treatment. The biochemical parameters such as plasma glucose (mg/dL), plasma triglyceride (mg/dL), plasma cholesterol (mg/dL), plasma AST (IU/L), plasma ALT (IU/L) and liver weight (g) may be recorded at the end of 14 days.

### Example 595

### Chronic Study 2

### Effect of the Test Compound on High Fat Diet Induced Weight Gain in Wistar Rats

### Meal-Fed Protocol

Male Wistar rat mice aged 4 weeks with body weight range of 150-180 g are procured from the central animal house facility, Piramal Life Sciences Limited, Mumbai, India. Animals are housed in individually ventilated cages (IVC's) at a room temperature of 22±2° C., humidity 55±5% with a 12:12 h light-dark cycle and have access to water ad libitum. Rats (two/cage) are allowed to acclimatize on Standard diet (Normal Pellet Diet; NPD; Amrut Laboratory Animal Feed, India) for one week. Then, animals are grouped based on body weight and plasma glucose with similar mean±S.E.M. with 10 animals per group.

### Acclimatization Period

All the rats are housed individually in IVC's cages and subjected to 9 days acclimatization period. In brief, animals are provided with either NPD or high fat diet (HFD, D12492; Research Diets Inc., NJ, USA). Animals are provided with ad libitum feed form day 1 to day 3. From day 4 to day 6, food is restricted for 12 hours. From day 7 to day 9, food is provided for three hours in the morning and three hours in the evening. During acclimatization period, rats are administered with vehicle (1% Tween 80 in 0.5% CMC; 10 ml/kg) twice daily, to acclimatize them to oral dosing and handling procedures.

### Treatment Regimen

On day 10, high fat fed animals are regrouped to three groups based on body weight with similar mean±S.E.M. with 10 animals per group. The test compound is prepared as suspension with 1% Tween 80 in 0.5% CMC. Vehicle (0.5% CMC with 1% Tween 80; 10 mL/kg) or the test compound is administered twice daily in the morning and evening. The concentration of test compounds used is in the range of 1 to 10 mg/kg (p.o., b.i.d.). This dosing regimen is continued for 14 days. Daily body weight is recorded just before test compound administration.

### Food Intake Measurement

Food intake is measured twice daily. In the morning, random amount of LFD or HFD is kept in the metallic lid. It is weighed with food and is considered as food provided. At noon, lid weight with food is measured as food remaining. Food intake in morning is calculated as difference between food provided and food remaining. Mice are devoid of food for six hours. In the evening, again food is provided and food intake is measured at 9 pm as per the above procedure during morning session. Followed by this, food is removed from the cages for twelve hours. Sum of the food intake in the morning and in the evening gives total food intake during the corresponding day.

### Biochemical Parameters Estimation and Necropsy

Blood (˜80 μL) is collected from the retro-orbital plexus of rats on day 15, 1 h after administration of the test compound. The plasma is separated by centrifugation at 8000×g for 7 min at 4° C. and plasma glucose, triglyceride, cholesterol, liver enzymes (ALT and AST), LDL-C and HDL-C are estimated immediately using a biochemistry autoanalyser (Hitachi Science Systems Limited, Ibaraki, Japan). Plasma insulin (Linco Research, USA) is estimated as per manufacturer's protocol.

Observations are recorded for percent change in body weight gain and cumulative feed intake during 14 days of treatment. The biochemical parameters such as plasma glucose (mg/dL), plasma triglyceride (mg/dL), plasma cholesterol (mg/dL), plasma AST (IU/L), plasma ALT (IU/L) and liver weight (g) may be recorded at the end of 14 days.

### Example 596

### Chronic Study 3

### Effect of Test Compound on High Fat Diet Induced Hyperlipidemia in Hamster

### Protocol

Male hamsters aged 9-10 weeks with body weight range of 90-110 g are procured from the central animal house facility, Piramal Life Sciences Limited, Mumbai, India. Animals are housed in individually ventilated cages (IVC's) at a room temperature of 22±2° C., humidity 55±5% with a 12:12 h light-dark cycle and have access to water ad libitum. Hamsters (two/cage) are allowed to acclimatize on standard diet (normal pellet diet, NPD; Amrut Laboratory Animal Feed, India) for one week. Animals are then grouped based on plasma triglyceride and cholesterol with similar mean±S.E.M. with 10 animals per group.

### Diet

Animals are provided with high cholesterol high fat diet (HCHF). HCHF is prepared in-house (cholesterol 1%, fructose 10%, coconut oil 25%, corn starch 5% and made to 100% by NPD) and is provided ad libitum for all the 14 days.

### Treatment Regimen

The test compound is prepared as suspension with 1% Tween 80 in 0.5% CMC. Vehicle (0.5% CMC with 1% Tween 80; 10 mL/kg) or test compound are administered twice daily in the morning and evening. The concentration of test compounds used is in the range of 1 to 10 mg/kg (p.o., b.i.d.). This dosing regimen is continued for 14 days. Daily body weight is recorded just before test compound administration.

### Biochemical Parameters Estimation and Necropsy

Blood (˜80 μL) is collected from the retro-orbital plexus of hamster on day 15. Plasma is separated by centrifugation at 8000×g for 7 min at 4° C. and plasma glucose, triglyceride, cholesterol, liver enzymes (ALT and AST), LDL-C and HDL-C are estimated immediately using a biochemistry autoanalyser (Hitachi Science Systems Limited, Ibaraki, Japan). Plasma insulin (Linco Research, USA) is estimated as per manufacturer's protocol.

Observations are recorded for percent change in body weight gain and cumulative feed intake during 14 days of treatment. The biochemical parameters such as plasma glucose (mg/dL), plasma triglyceride (mg/dL), plasma cholesterol (mg/dL), plasma AST (IU/L), plasma ALT (IU/L) and liver weight (g) may be recorded at the end of 14 days.

### Example 597

### Acute Study 1

### Effect of Test Compound on Feed Intake in Sprague Dawley Rats Fed on High Fat Diet

### Protocol

Male Sprague Dawley rat aged 5-6 weeks with body weight range of 200-220 g are procured from the central animal house facility, Piramal Life Sciences Limited, Mumbai, India. Animals are housed in individually ventilated cages (IVC's) at a room temperature of 22±2° C., humidity 55±5% with a 12:12 h light-dark cycle and have access to water ad libitum. After a 12 h fasting period, animals are grouped based on body weight with similar mean±S.E.M. with 9 animals per group.

### Treatment

The test compound is prepared as suspension with 1% Tween 80 in 0.5% CMC. Vehicle (0.5% CMC with 1% Tween 80; 10 mL/kg) or test compound are administered in the morning (9 am). The concentration of test compounds used is in the range of 1 to 10 mg/kg (p.o.). High Fat diet (HFD) is immediately provided to the animals after dosing. Food intake is measured at 1, 2, 4, 6 and 8 h post dose.

### Food Intake Measurement

Random amount of HFD is kept in the metallic lid. It is weighed with food and is considered as food provided. At 1, 2, 4, 6 and 8 h lid weight with food is measured as food remaining. Food intake is calculated as difference between food provided and food remaining.

### Percent Inhibition of Food Intake

Percentage inhibition is calculated separately for 1, 2, 4, 6 and 8 h. It is calculated with respect to HFD fed vehicle group using the formula % inhibition=(Mean feed intake of vehicle group of respective hour−feed intake of each animal in treatment group of respective hour)/Mean feed intake of vehicle group of respective hour×100.

It should be noted that, as used in this specification and the appended claims, the singular forms “a”, “an”, and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

All publications and patent applications in this specification are indicative of the level of ordinary skill in the art to which this invention pertains.

The invention has been described with reference to various specific and preferred aspects and techniques. However, it should be understood that many variations and modifications may be made while remaining within the spirit and scope of the invention.

1-67. (canceled)

68. A compound of formula 1: 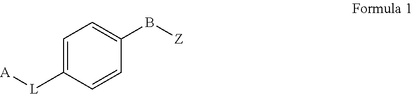
or a stereoisomer, a tautomer, a pharmaceutically acceptable salt, or N-oxide thereof;
wherein,
Z is selected from: 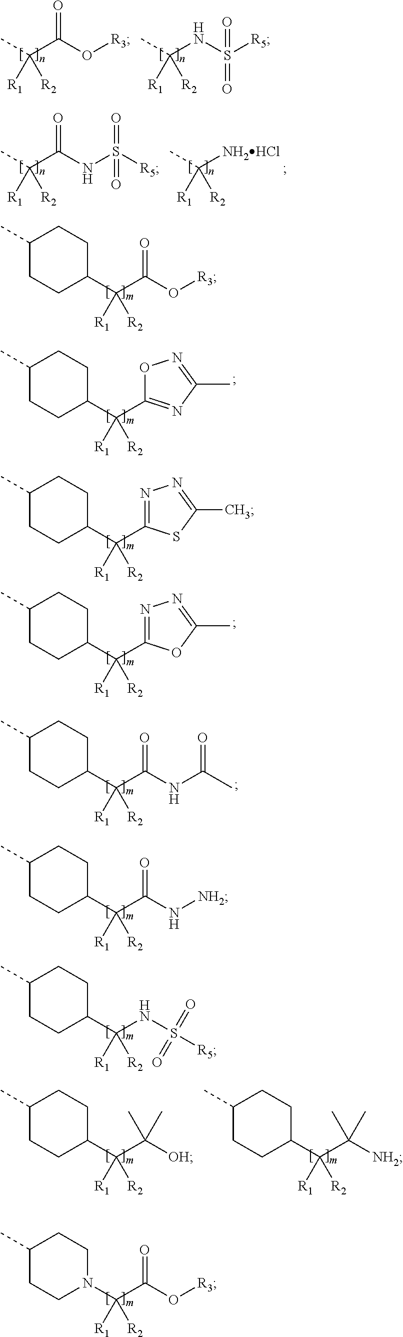 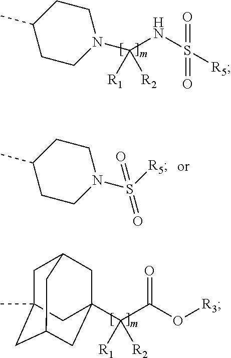 [symbol]
indicates the point of attachment;
n is an integer selected from 1-5;
m is 0 or 1;
R₁ and R₂ are independently selected from hydrogen or (C₁-C₁₂)-alkyl, or R₁ and R₂ can optionally form an unsubstituted or substituted (C₃-C₇) cycloalkyl ring;
R₃ is hydrogen or (C₁-C₁₂)-alkyl;
R₅ is selected from hydrogen, (C₁-C₁₂)-alkyl, CF₃, (C₃-C₇)-cycloalkyl, aryl, or heterocyclyl,
B is a 5-membered heteroaryl ring represented by any one of the general structures (i) to (x); 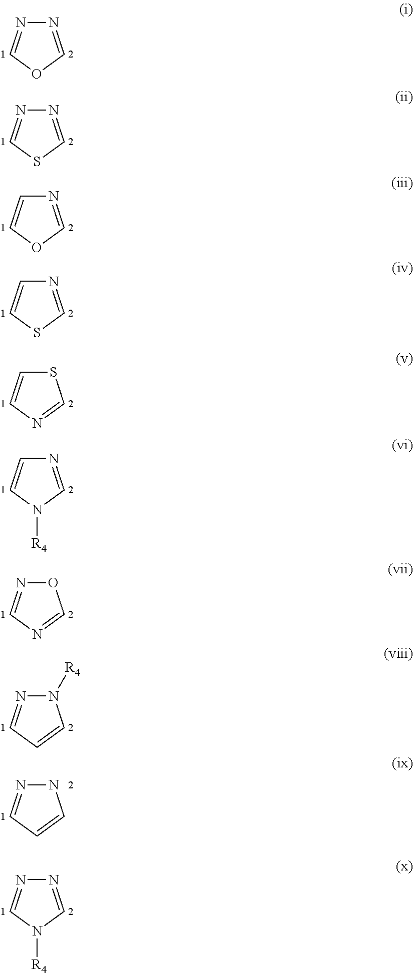
wherein 1 and 2 are the points of attachment of B to phenyl and to Z respectively and R₄ is selected from hydrogen, (C₁-C₁₂)-alkyl or aryl; or
B is a 6-membered heteroaryl ring containing 1 or 2 N-atoms, wherein the 6-membered heteroaryl ring may be unsubstituted or substituted with one or more groups selected from halogen, hydroxy, (C₁-C₁₂)-alkoxy, cyano, nitro, (C₁-C₁₂)-alkyl, (C₂-C₁₂)-alkenyl, (C₂-C₁₂)-alkynyl, (C₃-C₁₂)-cycloalkyl, aryl, aryloxy, heterocyclyl or O-heterocyclyl;
L is selected from *NHC(O)NH, *N(CH₃)C(O)NH *NHC(S)NH, *SO₂NH, *CONH or *NH(C═NR₆)NH, wherein * indicates the point of attachment of L to A, and R₆ is selected from hydrogen, methyl, cyano or nitro;
A is selected from (C₁-C₁₂)-alkyl, (C₃-C₁₂)-cycloalkyl, aryl or heterocyclyl;
wherein,
(C₁-C₁₂)-alkyl is unsubstituted or substituted with one or more groups selected from halogen, hydroxy, (C₁-C₁₂)-alkoxy, cyano, nitro, (C₃-C₁₂)-cycloalkyl, aryl, heterocyclyl, C(O)Rₚ, C(O)ORₚ, NRₚR_{q}, C(O)NRₚR_{q}, SRₚ, S(O)Rₚ or SO₂Rₚ;
(C₃-C₁₂)-cycloalkyl is unsubstituted or substituted with one or more groups selected from halogen, hydroxy, (C₁-C₁₂)-alkoxy, cyano, nitro, aryl, heterocyclyl, C(O)Rₚ C(O)ORₚ, NRₚR_{q}, C(O)NRₚR_{q}, SRₚ, S(O)Rₚ or SO₂Rₚ;
aryl is unsubstituted or substituted with one or more groups selected from halogen, hydroxy, (C₁-C₁₂)-alkoxy, cyano, nitro, (C₁-C₁₂)-alkyl, OCF₃, CF₃, (C₂-C₁₂)-alkenyl, (C₂-C₁₂)-alkynyl, (C₃-C₁₂)-cycloalkyl, aryl, aryloxy, heterocyclyl, O-heterocyclyl, C(O)Rₚ, C(O)ORₚ, NRₚR_{q}, C(O)NRₚR_{q}, SRₚ, S(O)Rₚ or SO₂Rₚ; or aryl may be fused with an unsubstituted or substituted 5- or 6-membered cycloalkyl ring optionally containing one or more heteroatoms selected from O, N or S;
heterocyclyl is unsubstituted or substituted with one or more groups selected from halogen, hydroxy, (C₁-C₁₂)-alkoxy, cyano, nitro, (C₁-C₁₂)-alkyl, (C₂-C₁₂)-alkenyl, (C₂-C₁₂)-alkynyl, (C₃-C₁₂)-cycloalkyl, aryl, aryloxy, heterocyclyl, O-heterocyclyl, C(O)Rₚ, C(O)ORₚ, NRₚR_{q}, C(O)NRₚR_{q}, SRₚ, S(O)Rₚ or SO₂Rₚ;
Rₚ and R_{q} are independently selected from hydrogen, (C₁-C₁₂)-alkyl, aryl, aralkyl or heterocyclyl, or Rₚ and R_{q} together with the N to which they are attached optionally form a 3- to 7-membered ring;
with a proviso that A is not a methyl group.

69. The compound according to claim 68, represented by a compound of formula 1a; 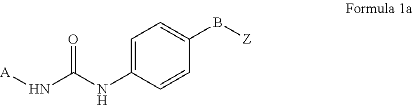
or a stereoisomer, a tautomer, a pharmaceutically acceptable salt, or N-oxide thereof;
wherein,
Z, B and A are as defined in claim 68.

70. The compound according to claim 69;
or a stereoisomer, a tautomer, a pharmaceutically acceptable salt, or N-oxide thereof;
wherein,
B is 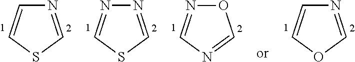
wherein 1 and 2 are the points of attachment of B to phenyl and to Z respectively;
Z and A are as defined in claim 68;
wherein,
(C₁-C₁₂)-alkyl is unsubstituted or substituted with one or more groups selected from halogen, hydroxy, (C₁-C₁₂)-alkoxy, cyano, (C₃-C₁₂)-cycloalkyl, aryl or heterocyclyl;
(C₃-C₁₂)-cycloalkyl is unsubstituted or substituted with one or more groups selected from halogen, hydroxy, (C₁-C₁₂)-alkoxy, cyano, aryl and heterocyclyl;
aryl is unsubstituted or substituted with one or more groups selected from halogen, hydroxy, (C₁-C₁₂)-alkoxy, cyano, (C₁-C₁₂)-alkyl, OCF₃, CF₃, (C₃-C₁₂)-cycloalkyl, aryl, aryloxy, heterocyclyl or O-heterocyclyl, or aryl may be fused with an unsubstituted or substituted 5 or 6-membered cycloalkyl ring optionally containing one or more heteroatoms selected from O, N or S;
heterocyclyl is unsubstituted or substituted with one or more groups selected from halogen, hydroxy, (C₁-C₁₂)-alkoxy, cyano, (C₁-C₁₂)-alkyl, (C₃-C₁₂)-cycloalkyl, aryl, aryloxy, heterocyclyl or O-heterocyclyl;
with the proviso that A is not a methyl group.

71. The compound according to claim 70;
or a stereoisomer, a tautomer, a pharmaceutically acceptable salt, or N-oxide thereof;
wherein,
B is 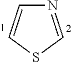
wherein 1 and 2 are the points of attachment of B to phenyl and to Z respectively.

72. The compound according to claim 70;
wherein,
B is 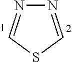
wherein 1 and 2 are the points of attachment of B to phenyl and to Z respectively.

73. The compound according to claim 70;
or a stereoisomer, a tautomer, a pharmaceutically acceptable salt, or N-oxide thereof;
wherein,
B is 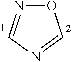
wherein 1 and 2 are the points of attachment of B to phenyl and to Z respectively.

74. The compound according to claim 70,
or a stereoisomer, a tautomer, a pharmaceutically acceptable salt, or N-oxide thereof;
wherein,
B is 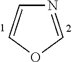
wherein 1 and 2 are the points of attachment of B to phenyl and to Z respectively.

75. The compound according to claim 68, represented by a compound of formula 1b, 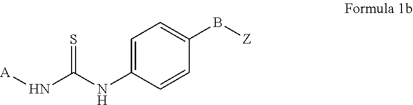
or a stereoisomer, tautomer, pharmaceutically acceptable salt, or N-oxide thereof;
wherein,
Z, B and A are as defined in claim 68.

76. The compound according to claim 75;
or a stereoisomer, a tautomer, a pharmaceutically acceptable salt, or N-oxide thereof;
wherein,
B is 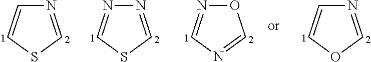
wherein 1 and 2 are the points of attachment of B to phenyl and to Z respectively;
Z and A are as defined in claim 68;
wherein,
(C₁-C₁₂)-alkyl is unsubstituted or substituted with one or more groups selected from halogen, hydroxy, (C₁-C₁₂)-alkoxy, cyano, (C₃-C₁₂)-cycloalkyl, aryl or heterocyclyl;
(C₃-C₁₂)-cycloalkyl is unsubstituted or substituted with one or more groups selected from halogen, hydroxy, (C₁-C₁₂)-alkoxy, cyano, aryl and heterocyclyl;
aryl is unsubstituted or substituted with one or more groups selected from halogen, hydroxy, (C₁-C₁₂)-alkoxy, cyano, (C₁-C₁₂)-alkyl, OCF₃, CF₃, (C₃-C₁₂)-cycloalkyl, aryl, aryloxy, heterocyclyl or O-heterocyclyl, or aryl may be fused with an unsubstituted or substituted 5 or 6-membered cycloalkyl ring optionally containing one or more heteroatoms selected from O, N or S;
heterocyclyl is unsubstituted or substituted with one or more groups selected from halogen, hydroxy, (C₁-C₁₂)-alkoxy, cyano, (C₁-C₁₂)-alkyl, (C₃-C₁₂)-cycloalkyl, aryl, aryloxy, heterocyclyl or O-heterocyclyl;
with the proviso that A is not a methyl group.

77. The compound according to claim 76;
or a stereoisomer, a tautomer, a pharmaceutically acceptable salt, or N-oxide thereof;
wherein,
B is 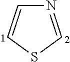
wherein 1 and 2 are the points of attachment of B to phenyl and to Z respectively.

78. The compound according to claim 76,
or a stereoisomer, a tautomer, a pharmaceutically acceptable salt, or N-oxide thereof;
wherein,
B is 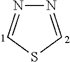
wherein 1 and 2 are the points of attachment of B to phenyl and to Z respectively.

79. The compound according to claim 76,
or a stereoisomer, a tautomer, a pharmaceutically acceptable salt, or N-oxide thereof;
wherein,
B is 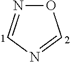
wherein 1 and 2 are the points of attachment of B to phenyl and to Z respectively.

80. The compound according to claim 76,
or a stereoisomer, a tautomer, a pharmaceutically acceptable salt, or N-oxide thereof;
wherein,
B is 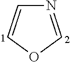
wherein 1 and 2 are the points of attachment of B to phenyl and to Z respectively.

81. The compound according to claim 68 represented by a compound of formula 1c, 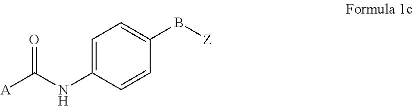
or a stereoisomer, tautomer, pharmaceutically acceptable salt or N-oxide thereof;
wherein,
Z, B and A are as defined in claim 68.

82. The compound according to claim 81,
or a stereoisomer, a tautomer, a pharmaceutically acceptable salt, or N-oxide thereof;
wherein,
B is 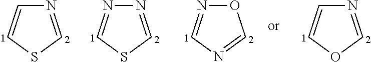
wherein 1 and 2 are the points of attachment of B to phenyl and to Z respectively;
Z and A are as defined in claim 68;
wherein,
(C₁-C₁₂)-alkyl is unsubstituted or substituted with one or more groups selected from halogen, hydroxy, (C₁-C₁₂)-alkoxy, cyano, (C₃-C₁₂)-cycloalkyl, aryl or heterocyclyl;
(C₃-C₁₂)-cycloalkyl is unsubstituted or substituted with one or more groups selected from halogen, hydroxy, (C₁-C₁₂)-alkoxy, cyano, aryl and heterocyclyl;
aryl is unsubstituted or substituted with one or more groups selected from halogen, hydroxy, (C₁-C₂)-alkoxy, cyano, (C₁-C₁₂)-alkyl, OCF₃, CF₃, (C₃-C₁₂)-cycloalkyl, aryl, aryloxy, heterocyclyl or O-heterocyclyl, or aryl may be fused with an unsubstituted or substituted 5 or 6-membered cycloalkyl ring optionally containing one or more heteroatoms selected from O, N or S;
heterocyclyl is unsubstituted or substituted with one or more groups selected from halogen, hydroxy, (C₁-C₁₂)-alkoxy, cyano, (C₁-C₁₂)-alkyl, (C₃-C₁₂)-cycloalkyl, aryl, aryloxy, heterocyclyl or O-heterocyclyl;
with the proviso that A is not a methyl group.

83. The compound according to claim 82,
or a stereoisomer, a tautomer, a pharmaceutically acceptable salt, or N-oxide thereof;
wherein,
B is 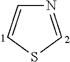
wherein 1 and 2 are the points of attachment of B to phenyl and to Z respectively.

84. The compound according to claim 82,
or a stereoisomer, a tautomer, a pharmaceutically acceptable salt, or N-oxide thereof;
wherein,
B is 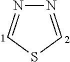
wherein 1 and 2 are the points of attachment of B to phenyl and to Z respectively.

85. The compound according to claim 82,
or a stereoisomer, a tautomer, a pharmaceutically acceptable salt, or N-oxide thereof;
wherein,
B is 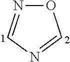
wherein 1 and 2 are the points of attachment of B to phenyl and to Z respectively.

86. The compound according to claim 82,
or a stereoisomer, a tautomer, a pharmaceutically acceptable salt, or N-oxide thereof;
wherein.
B is 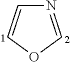
wherein 1 and 2 are the points of attachment of B to phenyl and to Z respectively.

87. The compound according to claim 68 represented by a compound of formula 1d, 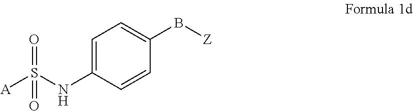
or a stereoisomer, tautomer, pharmaceutically acceptable salt or N-oxide thereof;
wherein,
Z, B and A are as defined in claim 68;
wherein,
(C₁-C₁₂)-alkyl is unsubstituted or substituted with one or more groups selected from halogen, hydroxy, (C₁-C₁₂)-alkoxy, cyano, (C₃-C₁₂)-cycloalkyl, aryl or heterocyclyl;
(C₃-C₁₂)-cycloalkyl is unsubstituted or substituted with one or more groups selected from halogen, hydroxy, (C₁-C₁₂)-alkoxy, cyano, aryl and heterocyclyl;
aryl is unsubstituted or substituted with one or more groups selected from halogen, hydroxy, (C₁-C₁₂)-alkoxy, cyano, (C₁-C₁₂)-alkyl, OCF₃, CF₃, (C₃-C₁₂)-cycloalkyl, aryl, aryloxy, heterocyclyl or O-heterocyclyl, or aryl may be fused with an unsubstituted or substituted 5 or 6-membered cycloalkyl ring optionally containing one or more heteroatoms selected from O, N or S;
heterocyclyl is unsubstituted or substituted with one or more groups selected from halogen, hydroxy, (C₁-C₁₂)-alkoxy, cyano, (C₁-C₁₂)-alkyl, (C₃-C₁₂)-cycloalkyl, aryl, aryloxy, heterocyclyl or O-heterocyclyl;
with the proviso that A is not a methyl group.

88. The compound according to claim 68, represented by a compound of formula 1e, 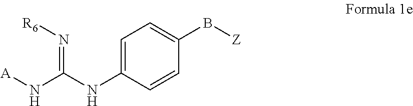
or a stereoisomer, tautomer, pharmaceutically acceptable salt or N-oxide thereof;
wherein,
Z, B and A are as defined in claim 68;
wherein,
(C₁-C₁₂)-alkyl is unsubstituted or substituted with one or more groups selected from halogen, hydroxy, (C₁-C₁₂)-alkoxy, cyano, (C₃-C₁₂)-cycloalkyl, aryl or heterocyclyl;
(C₃-C₁₂)-cycloalkyl is unsubstituted or substituted with one or more groups selected from halogen, hydroxy, (C₁-C₁₂)-alkoxy, cyano, aryl and heterocyclyl;
aryl is unsubstituted or substituted with one or more groups selected from halogen, hydroxy, (C₁-C₁₂)-alkoxy, cyano, (C₁-C₁₂)-alkyl, OCF₃, CF₃, (C₃-C₁₂)-cycloalkyl, aryl, aryloxy, heterocyclyl or O-heterocyclyl, or aryl may be fused with an unsubstituted or substituted 5 or 6-membered cycloalkyl ring optionally containing one or more heteroatoms selected from O, N or S;
heterocyclyl is unsubstituted or substituted with one or more groups selected from halogen, hydroxy, (C₁-C₁₂)-alkoxy, cyano, (C₁-C₁₂)-alkyl, (C₃-C₁₂)-cycloalkyl, aryl, aryloxy, heterocyclyl or O-heterocyclyl;
with the proviso that A is not a methyl group.

89. A compound according to claim 68, wherein A is an aryl and said aryl is unsubstituted or substituted with one or more groups selected from halogen, hydroxy, (C₁-C₁₂)-alkoxy, cyano, (C₁-C₁₂)-alkyl, OCF₃, CF₃, (C₃-C₁₂)-cycloalkyl, aryl, aryloxy, heterocyclyl, or O-heterocyclyl.

90. A compound according to claim 68, wherein A is an aryl group and said aryl group may be fused with an unsubstituted or substituted 5 or 6-membered cycloalkyl ring optionally containing one or more heteroatoms selected from O, N or S.

91. A compound according to claim 68, wherein A is a heterocyclyl and said heterocyclyl is unsubstituted or substituted with one or more groups selected from halogen, hydroxy, (C₁-C₁₂)-alkoxy, cyano, (C₁-C₁₂)-alkyl, (C₃-C₁₂)-cycloalkyl, aryl, aryloxy, heterocyclyl or O-heterocyclyl.

92. A compound according to claim 68, wherein A is a (C₃-C₁₂)-cycloalkyl and said (C₃-C₁₂)-cycloalkyl is unsubstituted or substituted with one or more groups selected from halogen, hydroxy, (C₁-C₁₂)-alkyl, (C₁-C₁₂)-alkoxy, cyano, nitro, aryl or heterocyclyl.

93. A compound according to claim 68, wherein A is an (C₁-C₁₂)-alkyl and said (C₁-C₁₂)-alkyl is unsubstituted or substituted with one or more groups selected from halogen, hydroxy, (C₁-C₁₂)-alkoxy, cyano, (C₃-C₁₂)-cycloalkyl, aryl or heterocyclyl; with the proviso that A is not a methyl group.

94. The compound according to claim 68 selected from:
Methyl 3-(5-(4-(3-(3-(trifluoromethyl)phenyl)ureido)phenyl)thiazol-2-yl)propanoate;
3-(5-(4-(3-(3-(Trifluoromethyl)phenyl)ureido)phenyl)thiazol-2-yl)propanoic acid;
Methyl 3-(5-(4-(3-(2-chlorophenyl)ureido)phenyl)thiazol-2-yl)propanoate;
3-(5-(4-(3-(2-Chlorophenyl)ureido)phenyl)thiazol-2-yl)propanoic acid;
Methyl 3-(5-(4-(3-cyclohexylureido)phenyl)thiazol-2-yl)propanoate;
3-(5-(4-(3-Cyclohexylureido)phenyl)thiazol-2-yl)propanoic acid;
Methyl 3-(5-(4-(3-(4-chloro-2-phenoxyphenyl)ureido)phenyl)thiazol-2-yl)propanoate;
3-(5-(4-(3-(4-Chloro-2-phenoxyphenyl)ureido)phenyl)thiazol-2-yl)propanoic acid;
Methyl 3-(5-(4-(4-t-butylbenzamido)phenyl)thiazol-2-yl)propanoate;
3-(5-(4-(4-t-Butylbenzamido)phenyl)thiazol-2-yl)propanoic acid;
Methyl 3-(5-(4-(4-pentylbenzamido)phenyl)thiazol-2-yl)propanoate;
3-(5-(4-(4-Pentylbenzamido)phenyl)thiazol-2-yl)propanoic acid;
Methyl 3-(5-(4-(3-ethoxy-5-(methoxymethyl)benzamido)phenyl)thiazol-2-yl)propanoate;
3-(5-(4-(3-Ethoxy-5-(methoxymethyl)benzamido)phenyl)thiazol-2-yl)propanoic acid;
Methyl 3-(5-(4-(4-pentylbenzamido)phenyl)thiazol-2-yl)propanoate;
3-(5-(4-(2-Naphthamido)phenyl)thiazol-2-yl)propanoic acid;
Methyl 3-(5-(4-(4-butoxybenzamido)phenyl)thiazol-2-yl)propanoate;
3-(5-(4-(4-Butoxybenzamido)phenyl)thiazol-2-yl)propanoic acid;
Methyl 3-(5-(4-(2,4-dimethoxyphenylsulfonamido)phenyl)thiazol-2-yl)propanoate;
3-(5-(4-(2,4-Dimethoxyphenylsulfonamido)phenyl)thiazol-2-yl)propanoic acid;
Methyl 3-(5-(4-(3-(2-chlorophenyl)ureido)phenyl)thiazol-2-yl)-2,2-dimethyl propanoate;
3-(5-(4-(3-(2-Chlorophenyl)ureido)phenyl)thiazol-2-yl)-2,2-dimethylpropanoic acid;
Methyl 2,2-dimethyl-3-(5-(4-(3-(4-(trifluoromethyl)phenyl)ureido)phenyl)thiazol-2-yl)propanoate;
2,2-Dimethyl-3-(5-(4-(3-(4-(trifluoromethyl)phenyl)ureido)phenyl)thiazol-2-yl)propanoic acid;
Methyl 2,2-dimethyl-3-(5-(4-(3-(4-(trifluoromethyl)phenyl)ureido)phenyl)thiazol-2-yl)propanoate;
3-(5-(4-(3-(4-Fluorophenyl)ureido)phenyl)thiazol-2-yl)-2,2-dimethylpropanoic acid;
Methyl 3-(5-(4-(3-(4-methoxyphenyl)ureido)phenyl)thiazol-2-yl)-2,2-dimethylpropanoate;
3-(5-(4-(3-(4-Methoxyphenyl)ureido)phenyl)thiazol-2-yl)-2,2-dimethylpropanoic acid;
Methyl 3-(5-(4-(3-cyclohexylureido)phenyl)thiazol-2-yl)-2,2-dimethyl propanoate;
3-(5-(4-(3-Cyclohexylureido)phenyl)thiazol-2-yl)-2,2-dimethyl propanoic acid;
Methyl 3-(5-(4-(3-(4-chloro-2-phenoxyphenyl)ureido)phenyl)thiazol-2-yl)-2,2-dimethylpropanoate;
3-(5-(4-(3-(4-Chloro-2-phenoxyphenyl)ureido)phenyl)thiazol-2-yl)-2,2-dimethylpropanoic acid;
Methyl 3-(5-(4-(4-tert-butylbenzamido)phenyl)thiazol-2-yl)-2,2-dimethylpropanoate;
3-(5-(4-(4-t-Butylbenzamido)phenyl)thiazol-2-yl)-2,2-dimethylpropanoic acid;
Methyl 3-(5-(4-biphenyl-4-ylcarboxamidophenyl)thiazol-2-yl)-2,2-dimethylpropanoate;
3-(5-(4-Biphenyl-4-ylcarboxamidophenyl)thiazol-2-yl)-2,2-dimethylpropanoic acid;
Methyl 4-(5-(4-(3-(3-(trifluoromethyl)phenyl)ureido)phenyl)thiazol-2-yl)butanoate;
4-(5-(4-(3-(3-(Trifluoromethyl)phenyl)ureido)phenyl)thiazol-2-yl)butanoic acid;
Methyl 4-(5-(4-(3-(2-chlorophenyl)ureido)phenyl)thiazol-2-yl)butanoate;
4-(5-(4-(3-(2-Chlorophenyl)ureido)phenyl)thiazol-2-yl)butanoic acid;
Methyl 4-(5-(4-(3-(3,4-dimethylphenyl)ureido)phenyl)thiazol-2-yl)butanoate;
4-(5-(4-(3-(3,4-Dimethylphenyl)ureido)phenyl)thiazol-2-yl)butanoic acid;
Methyl 4-(5-(4-(3-(4-chloro-2-phenoxyphenyl)ureido)phenyl)thiazol-2-yl)butanoate;
4-(5-(4-(3-(4-Chloro-2-phenoxyphenyl)ureido)phenyl)thiazol-2-yl)butanoic acid;
Methyl 4-(5-(4-(4-t-butylbenzamido)phenyl)thiazol-2-yl)butanoate;
4-(5-(4-(4-t-Butylbenzamido)phenyl)thiazol-2-yl)butanoic acid;
Methyl 4-(5-(4-(4-pentylbenzamido)phenyl)thiazol-2-yl)butanoate;
4-(5-(4-(4-Pentylbenzamido)phenyl)thiazol-2-yl)butanoic acid;
Methyl 4-(5-(4-biphenyl-4-ylcarboxamidophenyl)thiazol-2-yl)butanoate;
4-(5-(4-Biphenyl-4-ylcarboxamidophenyl)thiazol-2-yl)butanoic acid;
Methyl 4-(5-(4-(2,4-Dimethoxyphenylsulfonamido)phenyl)thiazol-2-yl)butanoate;
4-(5-(4-(2,4-Dimethoxyphenylsulfonamido)phenyl)thiazol-2-yl)butanoic acid;
Methyl 3,3-dimethyl-4-(5-(4-(3-(3-(trifluoromethyl)phenyl)ureido)phenyl)thiazol-2-yl)butanoate;
3,3-Dimethyl-4-(5-(4-(3-(3-(trifluoromethyl)phenyl)ureido)phenyl)thiazol-2-yl)butanoic acid;
Methyl 4-(5-(4-(3-(2-chlorophenyl)ureido)phenyl)thiazol-2-yl)-3,3-dimethyl butanoate;
4-(5-(4-(3-(2-Chlorophenyl)ureido)phenyl)thiazol-2-yl)-3,3-dimethylbutanoic acid;
Methyl 4-(5-(4-(3-(4-chloro-2-phenoxyphenyl)ureido)phenyl)thiazol-2-yl)-3,3-dimethylbutanoate;
4-(5-(4-(3-(4-Chloro-2-phenoxyphenyl)ureido)phenyl)thiazol-2-yl)-3,3-dimethyl butanoic acid;
Methyl 4-(5-(4-(4-tert-butylbenzamido)phenyl)thiazol-2-yl)-3,3-dimethyl butanoate;
4-(5-(4-(4-t-Butylbenzamido)phenyl)thiazol-2-yl)-3,3-dimethylbutanoic acid;
Methyl 4-(5-(4-biphenyl-4-ylcarboxamidophenyl)thiazol-2-yl)-3,3-dimethyl butanoate;
4-(5-(4-Biphenyl-4-ylcarboxamidophenyl)thiazol-2-yl)-3,3-dimethylbutanoic acid;
Methyl 3,3-dimethyl-4-(5-(4-(4-pentylbenzamido)phenyl)thiazol-2-yl)butanoate;
3,3-Dimethyl-4-(5-(4-(4-pentylbenzamido)phenyl)thiazol-2-yl)butanoic acid;
Methyl 4-(5-(4-(2,4-dimethoxyphenylsulfonamido)phenyl)thiazol-2-yl)-3,3-dimethylbutanoate;
4-(5-(4-(2,4-Dimethoxyphenylsulfonamido)phenyl)thiazol-2-yl)-3,3-dimethylbutanoic acid;
Methyl 2,2-dimethyl-4-(5-(4-(3-(3-(trifluoromethyl)phenyl)ureido)phenyl)thiazol-2-yl)butanoate;
2,2-Dimethyl-4-(5-(4-(3-(3-(trifluoromethyl)phenyl)ureido)phenyl)thiazol-2-yl)butanoic acid;
Methyl 4-(5-(4-(3-(2-chlorophenyl)ureido)phenyl)thiazol-2-yl)-2,2-dimethyl butanoate;
4-(5-(4-(3-(2-Chlorophenyl)ureido)phenyl)thiazol-2-yl)-2,2-dimethylbutanoic acid;
Methyl 4-(5-(4-(3-(4-chloro-2-phenoxyphenyl)ureido)phenyl)thiazol-2-yl)-2,2-dimethylbutanoate;
4-(5-(4-(3-(4-Chloro-2-phenoxyphenyl)ureido)phenyl)thiazol-2-yl)-2,2-dimethylbutanoic acid;
Methyl 4-(5-(4-(3-cyclohexylureido)phenyl)thiazol-2-yl)-2,2-dimethyl butanoate;
4-(5-(4-(3-Cyclohexylureido)phenyl)thiazol-2-yl)-2,2-dimethylbutanoic acid;
Methyl 4-(5-(4-(3-(4-fluorophenyl)ureido)phenyl)thiazol-2-yl)y-2,2-dimethyl butanoate;
4-(5-(4-(3-(2-Chlorophenyl)ureido)phenyl)thiazol-2-yl)-2,2-dimethylbutanoic acid;
Methyl 4-(5-(4-(3-(4-methoxyphenyl)ureido)phenyl)thiazol-2-yl)-2,2-dimethyl butanoate;
4-(5-(4-(3-(4-Methoxyphenyl)ureido)phenyl)thiazol-2-yl)-2,2-dimethyl butanoic acid;
Methyl 4-(5-(4-(3-(4-isopropylphenyl)ureido)phenyl)thiazol-2-yl)-2,2-dimethyl butanoate;
4-(5-(4-(3-(4-Isopropylphenyl)ureido)phenyl)thiazol-2-yl)-2,2-dimethyl butanoic acid;
Methyl 4-(5-(4-(3-(2,4-difluorophenyl)ureido)phenyl)thiazol-2-yl)-2,2-dimethylbutanoate;
4-(5-(4-(3-(2,4-Difluorophenyl)ureido)phenyl)thiazol-2-yl)-2,2-dimethyl butanoic acid;
Methyl 4-(5-(4-(3-(2-fluorophenyl)ureido)phenyl)thiazol-2-yl)-2,2-dimethyl butanoate;
4-(4-(4-(3-(2-Fluorophenyl)ureido)phenyl)-3H-pyrol-2-yl)-2,2-dimethyl butanoic acid;
Methyl 4-(5-(4-(4-t-butylbenzamido)phenyl)thiazol-2-yl)-2,2-dimethyl butanoate;
4-(5-(4-(4-t-Butylbenzamido)phenyl)thiazol-2-yl)-2,2-dimethylbutanoic acid;
Methyl 4-(5-(4-biphenyl-4-ylcarboxamidophenyl)thiazol-2-yl)-2,2-dimethyl butanoate;
4-(5-(4-Biphenyl-4-ylcarboxamidophenyl)thiazol-2-yl)-2,2-dimethylbutanoic acid;
Methyl 2,2-dimethyl-4-(5-(4-(4-(oxazol-5-yl)benzamido)phenyl)thiazol-2-yl)butanoate;
2,2-Dimethyl-4-(5-(4-(4-(oxazol-5-yl)benzamido)phenyl)thiazol-2-yl)butanoic acid;
Methyl 2,2-dimethyl-4-(5-(4-(4-phenylthiazole-2-carboxamido)phenyl)thiazol-2-yl)butanoate;
2,2-Dimethyl-4-(5-(4-(4-phenylthiazole-2-carboxamido)phenyl)thiazol-2-yl)butanoic acid;
Methyl 3-(5-(4-(3-(2-chlorophenyl)ureido)phenyl)oxazol-2-yl)-2,2-dimethyl propanoate;
3-(5-(4-(3-(2-Chlorophenyl)ureido)phenyl)oxazol-2-yl)-2,2-dimethylpropanoic acid;
Methyl 2,2-dimethyl-3-(5-(4-(3-(4-(trifluoromethyl)phenyl)ureido)phenyl)oxazol-2-yl)propanoate;
2,2-Dimethyl-3-(5-(4-(3-(4-(trifluoromethyl)phenyl)ureido)phenyl)oxazol-2-yl)propanoic acid;
Methyl 3-(5-(4-(3-(4-fluorophenyl)ureido)phenyl)oxazol-2-yl)-2,2-dimethyl propanoate;
3-(5-(4-(3-(4-Fluorophenyl)ureido)phenyl)oxazol-2-yl)-2,2-dimethylpropanoic acid;
Methyl 3-(5-(4-(3-(4-methoxyphenyl)ureido)phenyl)oxazol-2-yl)-2,2-dimethyl propanoate;
3-(5-(4-(3-(4-Methoxyphenyl)ureido)phenyl)oxazol-2-yl)-2,2-dimethyl propanoic acid;
Methyl 3-(5-(4-(3-(4-chloro-2-phenoxyphenyl)ureido)phenyl)oxazol-2-yl)-2,2-dimethylpropanoate;
3-(5-(4-(3-(4-Chloro-2-phenoxyphenyl)ureido)phenyl)oxazol-2-yl)-2,2-dimethyl propanoic acid;
Methyl 3-(5-(4-(4-t-butylbenzamido)phenyl)oxazol-2-yl)-2,2-dimethyl propanoate;
3-(5-(4-(4-t-Butylbenzamido)phenyl)oxazol-2-yl)-2,2-dimethylpropanoic acid;
Methyl 3-(5-(4-biphenyl-4-ylcarboxamidophenyl)oxazol-2-yl)-2,2-dimethyl propanoate;
3-(5-(4-Biphenyl-4-ylcarboxamidophenyl)oxazol-2-yl)-2,2-dimethylpropanoic acid;
Methyl 4-(5-(4-(3-(3-(trifluoromethyl)phenyl)ureido)phenyl)thiazol-2-yl)cyclohexanecarboxylate;
4-(5-(4-(3-(3-(Trifluoromethyl)phenyl)ureido)phenyl)thiazol-2-yl)cyclohexane carboxylic acid;
Methyl 4-(5-(4-(3-p-tolylureido)phenyl)thiazol-2-yl)cyclohexanecarboxylate;
4-(5-(4-(3-p-Tolylureido)phenyl)thiazol-2-yl)cyclohexanecarboxylic acid;
Methyl 4-(5-(4-(3-(2,4-difluorophenyl)ureido)phenyl)thiazol-2-yl)cyclohexane carboxylate;
4-(5-(4-(3-(2,4-Difluorophenyl)ureido)phenyl)thiazol-2-yl)cyclohexane carboxylic acid;
Methyl 4-(5-(4-(3-(2-fluorophenyl)ureido)phenyl)thiazol-2-yl)cyclohexane carboxylate;
4-(5-(4-(3-(2-Fluorophenyl)ureido)phenyl)thiazol-2-yl)cyclohexanecarboxylic acid;
Methyl 4-(5-(4-(3-cyclohexylureido)phenyl)thiazol-2-yl)cyclohexane carboxylate;
4-(5-(4-(3-Cyclohexylureido)phenyl)thiazol-2-yl)cyclohexanecarboxylic acid;
Methyl 4-(5-(4-(3-(3-chlorophenyl)ureido)phenyl)thiazol-2-yl)cyclohexane carboxylate;
4-(5-(4-(3-(3-Chlorophenyl)ureido)phenyl)thiazol-2-yl)cyclohexanecarboxylic acid;
Methyl 4-(5-(4-(3-(4-chlorophenyl)ureido)phenyl)thiazol-2-yl)cyclohexane carboxylate;
4-(5-(4-(3-(4-Chlorophenyl)ureido)phenyl)thiazol-2-yl)cyclohexanecarboxylic acid;
Methyl 4-(5-(4-(3-(2-chloro-4-(trifluoromethyl)phenyl)ureido)phenyl)thiazol-2-yl)cyclohexanecarboxylate;
4-(5-(4-(3-(2-Chloro-4-(trifluoromethyl)phenyl)ureido)phenyl)thiazol-2-yl)cyclohexanecarboxylic acid;
Methyl 4-(5-(4-(3-(2-chloro-5-methylphenyl)ureido)phenyl)thiazol-2-yl)cyclo hexanecarboxylate;
4-(5-(4-(3-(2-Chloro-5-methylphenyl)ureido)phenyl)thiazol-2-yl)cyclohexane carboxylic acid;
Methyl 4-(5-(4-(3-(3-chloro-2-fluorophenyl)ureido)phenyl)thiazol-2-yl)cyclo hexanecarboxylate;
4-(5-(4-(3-(3-Chloro-2-fluorophenyl)ureido)phenyl)thiazol-2-yl)cyclohexane carboxylic acid;
Methyl 4-(5-(4-(3-(4-methoxy-2-methylphenyl)ureido)phenyl)thiazol-2-yl)cyclohexanecarboxylate;
4-(5-(4-(3-(4-Methoxy-2-methylphenyl)ureido)phenyl)thiazol-2-yl)cyclo hexanecarboxylic acid;
Methyl 4-(5-(4-(3-benzo[d][1,3]dioxol-5-ylureido)phenyl)thiazol-2-yl)cyclo hexanecarboxylate;
4-(5-(4-(3-Benzo[d][1,3]dioxol-5-ylureido)phenyl)thiazol-2-yl)cyclohexane carboxylic acid;
Methyl 4-(5-(4-(3-(2-chloro-6-(trifluoromethyl)phenyl)ureido)phenyl)thiazol-2-yl)cyclohexanecarboxylate;
4-(5-(4-(3-(2-Chloro-6-(trifluoromethyl)phenyl)ureido)phenyl)thiazol-2-yl)cyclohexanecarboxylic acid;
Methyl 4-(5-(4-(3-(4-chloro-2-(trifluoromethyl)phenyl)ureido)phenyl)thiazol-2-yl)cyclohexanecarboxylate;
4-(5-(4-(3-(4-Chloro-2-(trifluoromethyl)phenyl)ureido)phenyl)thiazol-2-yl)cyclohexane carboxylic acid;
Methyl 4-(5-(4-(3-(2-chloro-6-methylphenyl)ureido)phenyl)thiazol-2-yl)cyclohexane carboxylate;
4-(5-(4-(3-(2-Chloro-6-methylphenyl)ureido)phenyl)thiazol-2-yl)cyclohexane carboxylic acid;
Methyl 4-(5-(4-(3-(5-chloro-2-methylphenyl)ureido)phenyl)thiazol-2-yl)cyclohexane carboxylate;
4-(5-(4-(3-(5-Chloro-2-methylphenyl)ureido)phenyl)thiazol-2-yl)cyclohexane carboxylic acid;
Methyl 4-(5-(4-(3-(2-(trifluoromethyl)phenyl)ureido)phenyl)thiazol-2-yl)cyclohexane carboxylate;
4-(5-(4-(3-(2-(Trifluoromethyl)phenyl)ureido)phenyl)thiazol-2-yl)cyclohexane carboxylic acid;
Methyl 4-(5-(4-(3-(2-(trifluoromethoxy)phenyl)ureido)phenyl)thiazol-2-yl)cyclohexane carboxylate;
4-(5-(4-(3-(2-(Trifluoromethoxy)phenyl)ureido)phenyl)thiazol-2-yl)cyclohexane carboxylic acid;
Methyl 4-(5-(4-(3-(4-phenoxyphenyl)ureido)phenyl)thiazol-2-yl)cyclohexane carboxylate;
4-(5-(4-(3-(4-Phenoxyphenyl)ureido)phenyl)thiazol-2-yl)cyclohexane carboxylic acid;
Methyl 4-(5-(4-(3-(4-chloro-2-fluorophenyl)ureido)phenyl)thiazol-2-yl)cyclo hexanecarboxylate;
4-(5-(4-(3-(4-Chloro-2-fluorophenyl)ureido)phenyl)thiazol-2-yl)cyclohexane carboxylic acid;
Methyl 4-(5-(4-(3-(2-fluoro-5-methylphenyl)ureido)phenyl)thiazol-2-yl)cyclo hexanecarboxylate;
4-(5-(4-(3-(2-Fluoro-5-methylphenyl)ureido)phenyl)thiazol-2-yl)cyclohexane carboxylic acid;
Methyl 4-(5-(4-(3-(2-fluoro-6-(trifluoromethyl)phenyl)ureido)phenyl)thiazol-2-yl)cyclohexanecarboxylate;
4-(5-(4-(3-(2-Fluoro-6-(trifluoromethyl)phenyl)ureido)phenyl)thiazol-2-yl)cyclohexanecarboxylic acid;
Methyl 4-(5-(4-(3-(3-fluorophenyl)ureido)phenyl)thiazol-2-yl)cyclohexane carboxylate;
4-(5-(4-(3-(3-Fluorophenyl)ureido)phenyl)thiazol-2-yl)cyclohexanecarboxylic acid;
Methyl 4-(5-(4-(3-(3,4-difluorophenyl)ureido)phenyl)thiazol-2-yl)cyclohexane carboxylate;
4-(5-(4-(3-(3,4-Difluorophenyl)ureido)phenyl)thiazol-2-yl)cyclohexane carboxylic acid;
Methyl 4-(5-(4-(3-(3,5-difluorophenyl)ureido)phenyl)thiazol-2-yl)cyclohexane carboxylate;
4-(5-(4-(3-(3,5-Difluorophenyl)ureido)phenyl)thiazol-2-yl)cyclohexane carboxylic acid;
Methyl 4-(5-(4-(3-(2,6-difluorophenyl)ureido)phenyl)thiazol-2-yl)cyclohexane carboxylate;
4-(5-(4-(3-(2,6-Difluorophenyl)ureido)phenyl)thiazol-2-yl)cyclohexane carboxylic acid;
Methyl 4-(5-(4-(3-(2,3,4-trifluorophenyl)ureido)phenyl)thiazol-2-yl)cyclo hexanecarboxylate;
4-(5-(4-(3-(2,3,4-Trifluorophenyl)ureido)phenyl)thiazol-2-yl)cyclohexane carboxylic acid;
Methyl 4-(5-(4-(3-(2-chlorophenyl)ureido)phenyl)thiazol-2-yl)cyclohexane carboxylate;
4-(5-(4-(3-(2-Chlorophenyl)ureido)phenyl)thiazol-2-yl)cyclohexanecarboxylic acid;
Methyl 4-(5-(4-(3-(4-chloro-2-phenoxyphenyl)ureido)phenyl)thiazol-2-yl)cyclohexanecarboxylate;
4-(5-(4-(3-(4-Chloro-2-phenoxyphenyl)ureido)phenyl)thiazol-2-yl)cyclohexane carboxylic acid;
Methyl 4-(5-(4-(3-phenylureido)phenyl)thiazol-2-yl)cyclohexanecarboxylate;
4-(5-(4-(3-Phenylureido)phenyl)thiazol-2-yl)cyclohexanecarboxylic acid;
Methyl 4-(5-(4-(4-t-butylbenzamido)phenyl)thiazol-2-yl)cyclohexane carboxylate;
4-(5-(4-(4-t-Butylbenzamido)phenyl)thiazol-2-yl)cyclohexanecarboxylic acid;
Methyl 4-(5-(4-(2-chlorobenzamido)phenyl)thiazol-2-yl)cyclohexane carboxylate;
4-(5-(4-(2-Chlorobenzamido)phenyl)thiazol-2-yl)cyclohexanecarboxylic acid;
Methyl 4-(5-(4-(5-phenyloxazole-2-carboxamido)phenyl)thiazol-2-yl)cyclohexane carboxylate;
4-(5-(4-(5-Phenyloxazole-2-carboxamido)phenyl)thiazol-2-yl)cyclohexane carboxylic acid;
Methyl 4-(5-(4-(3-(4-methoxyphenyl)thioureido)phenyl)thiazol-2-yl)cyclo hexanecarboxylate;
Methyl 4-(5-(4-(3-(4-chlorophenyl)thioureido)phenyl)thiazol-2-yl)cyclo hexanecarboxylate;
Methyl 4-(5-(4-(3-(2-chlorophenyl)ureido)phenyl)oxazol-2-yl)cyclohexane carboxylate;
4-(5-(4-(3-(2-Chlorophenyl)ureido)phenyl)oxazol-2-yl)cyclohexanecarboxylic acid;
Methyl 4-(5-(4-(3-phenylureido)phenyl)oxazol-2-yl)cyclohexanecarboxylate;
4-(5-(4-(3-Phenylureido)phenyl)oxazol-2-yl)cyclohexanecarboxylic acid;
Methyl 4-(5-(4-(3-(3-chlorophenyl)ureido)phenyl)oxazol-2-yl)cyclohexane carboxylate;
4-(5-(4-(3-(3-Chlorophenyl)ureido)phenyl)oxazol-2-yl)cyclohexanecarboxylic acid;
Methyl 4-(5-(4-(3-(2-methoxyphenyl)ureido)phenyl)oxazol-2-yl)cyclohexane carboxylate;
4-(5-(4-(3-(2-Methoxyphenyl)ureido)phenyl)oxazol-2-yl)cyclohexane carboxylic acid;
Methyl 4-(5-(4-(2-chlorobenzamido)phenyl)oxazol-2-yl)cyclohexane carboxylate;
4-(5-(4-(2-Chlorobenzamido)phenyl)oxazol-2-yl)cyclohexanecarboxylic acid;
Methyl 4-(5-(4-(4-t-butylbenzamido)phenyl)oxazol-2-yl)cyclohexane carboxylate;
4-(5-(4-(4-t-Butylbenzamido)phenyl)oxazol-2-yl)cyclohexanecarboxylic acid;
(1r,4r)-Methyl 4-(3-(4-(3-(2-chlorophenyl)ureido)phenyl)-1,2,4-oxadiazol-5-yl)cyclohexanecarboxylate;
(1r,4r)-4-(3-(4-(3-(2-Chlorophenyl)ureido)phenyl)-1,2,4-oxadiazol-5-yl)cyclo hexanecarboxylic acid;
(1r,4r)-Methyl 4-(3-(4-(3-(2,4-difluorophenyl)ureido)phenyl)-1,2,4-oxadiazol-5-yl)cyclohexanecarboxylate;
(1r,4r)-4-(3-(4-(3-(2,4-Difluorophenyl)ureido)phenyl)-1,2,4-oxadiazol-5-yl)cyclohexanecarboxylic acid;
(1r,4r)-Methyl 4-(3-(4-(3-p-tolylureido)phenyl)-1,2,4-oxadiazol-5-yl)cyclohexane carboxylate;
(1r,4r)-4-(3-(4-(3-p-Tolylureido)phenyl)-1,2,4-oxadiazol-5-yl)cyclohexane carboxylic acid;
(1r,4r)-Methyl 4-(3-(4-(3-(3-chlorophenyl)ureido)phenyl)-1,2,4-oxadiazol-5-yl)cyclo hexanecarboxylate;
(1r,4r)-4-(3-(4-(3-(3-Chlorophenyl)ureido)phenyl)-1,2,4-oxadiazol-5-yl)cyclo hexanecarboxylic acid;
(1r,4r)-Methyl 4-(3-(4-(3-(4-chloro-2-phenoxyphenyl)ureido)phenyl)-1,2,4-oxadiazol-5-yl)cyclohexanecarboxylate;
(1r,4r)-4-(3-(4-(3-(4-Chloro-2-phenoxyphenyl)ureido)phenyl)-1,2,4-oxadiazol-5-yl)cyclohexanecarboxylic acid;
(1r,4r)-Methyl 4-(3-(4-(4-tert-butylbenzamido)phenyl)-1,2,4-oxadiazol-5-yl)cyclohexanecarboxylate;
(1r,4r)-4-(3-(4-(4-t-Butylbenzamido)phenyl)-1,2,4-oxadiazol-5-yl)cyclohexane carboxylic acid;
(1r,4r)-Methyl 4-(3-(4-biphenyl-4-ylcarboxamidophenyl)-1,2,4-oxadiazol-5-yl)cyclohexanecarboxylate;
(1r,4r)-4-(3-(4-Biphenyl-4-ylcarboxamidophenyl)-1,2,4-oxadiazol-5-yl)cyclo hexanecarboxylic acid;
(1r,4r-Methyl 4-(3-(4-(4-(trifluoromethoxy)benzamido)phenyl)-1,2,4-oxadiazol-5-yl)cyclohexanecarboxylate;
(1r,4r)-4-(3-(4-(4-(Trifluoromethoxy)benzamido)phenyl)-1,2,4-oxadiazol-5-yl)cyclohexanecarboxylic acid;
Methyl 4-(5-(4-(3-(3,5-difluorophenyl)ureido)phenyl)thiazol-2-yl)-2,2-dimethyl butanoate;
4-(5-(4-(3-(3,5-Difluorophenyl)ureido)phenyl)thiazol-2-yl)-2,2-dimethylbutanoic acid;
Sodium salt of 4-(5-(4-(3-(3,5-difluorophenyl)ureido)phenyl)thiazol-2-yl)-2,2-dimethylbutanoate;
Methyl 2,2-dimethyl-4-(5-(4-(3-(2,4,5-trifluorophenyl)ureido)phenyl)thiazol-2-yl)butanoate;
2,2-Dimethyl-4-(5-(4-(3-(2,4,5-trifluorophenyl)ureido)phenyl)thiazol-2-yl)butanoic acid;
Sodium salt of 2,2-dimethyl-4-(5-(4-(3-(2,4,5-trifluorophenyl)ureido)phenyl)thiazol-2-yl)butanoate;
Methyl 2,2-dimethyl-4-(5-(4-(piperidine-1-carboxamido)phenyl)thiazol-2-yl)butanoate;
2,2-Dimethyl-4-(5-(4-(piperidine-1-carboxamido)phenyl)thiazol-2-yl)butanoic acid;
Methyl 2,2-dimethyl-4-(5-(4-(morpholine-4-carboxamido)phenyl)thiazol-2-yl)butanoate;
2,2-Dimethyl-4-(5-(4-(morpholine-4-carboxamido)phenyl)thiazol-2-yl)butanoic acid;
Methyl 2,2-dimethyl-4-(5-(4-(4-methylpiperazine-1-carboxamido)phenyl)thiazol-2-yl)butanoate;
2,2-Dimethyl-4-(5-(4-(4-methylpiperazine-1-carboxamido)phenyl)thiazol-2-yl)butanoic acid hydrochloride;
Methyl 4-(5-(4-(3-(2,3-dihydrobenzo[b][1,4]dioxin-6-yl)ureido)phenyl)thiazol-2-yl)-2,2-dimethylbutanoate;
4-(5-(4-(3-(2,3-Dihydrobenzo[b][1,4]dioxin-6-yl)ureido)phenyl)thiazol-2-yl)-2,2-dimethylbutanoic acid;
Methyl 4-(5-(4-(3-(1H-tetrazol-5-yl)ureido)phenyl)thiazol-2-yl)-2,2-dimethyl butanoate;
4-(5-(4-(3-(1H-Tetrazol-5-yl)ureido)phenyl)thiazol-2-yl)-2,2-dimethylbutanoic acid;
Methyl 4-(5-(4-(3-(2-methoxyethyl)ureido)phenyl)thiazol-2-yl)-2,2-dimethyl butanoate;
4-(5-(4-(3-(2-Methoxyethyl)ureido)phenyl)thiazol-2-yl)-2,2-dimethylbutanoic acid;
Methyl 4-(5-(4-(3-(2,3-dihydro-1H-inden-2-yl)ureido)phenyl)thiazol-2-yl)-2,2-dimethylbutanoate;
4-(5-(4-(3-(2,3-Dihydro-1H-inden-2-yl)ureido)phenyl)thiazol-2-yl)-2,2-dimethylbutanoic acid;
Methyl 4-(5-(4-(3-cyclohexyl-3-methylureido)phenyl)thiazol-2-yl)-2,2-dimethyl butanoate;
4-(5-(4-(3-Cyclohexyl-3-methylureido)phenyl)thiazol-2-yl)-2,2-dimethylbutanoic acid;
Methyl 2,2-dimethyl-4-(5-(4-(3-(3,4,5-trifluorophenyl)ureido)phenyl)thiazol-2-yl)butanoate;
2,2-Dimethyl-4-(5-(4-(3-(3,4,5-trifluorophenyl)ureido)phenyl)thiazol-2-yl)butanoic acid;
Sodium salt of 2,2-dimethyl-4-(5-(4-(3-(3,4,5-trifluorophenyl)ureido) phenyl)thiazol-2-yl)butanoate;
Methyl 2,2-dimethyl-4-(5-(4-(3-(2-(piperidin-1-yl)ethyl)ureido)phenyl)thiazol-2-yl)butanoate;
2,2-Dimethyl-4-(5-(4-(3-(2-(piperidin-1-yl)ethyl)ureido)phenyl)thiazol-2-yl)butanoic acid;
Methyl 4-(5-(4-(3-benzylureido)phenyl)thiazol-2-yl)-2,2-dimethylbutanoate;
4-(5-(4-(3-Benzylureido)phenyl)thiazol-2-yl)-2,2-dimethylbutanoic acid;
Methyl 4-(5-(4-(4,4-difluoropiperidine-1-carboxamido)phenyl)thiazol-2-yl)-2,2-dimethylbutanoate;
4-(5-(4-(4,4-Difluoropiperidine-1-carboxamido)phenyl)thiazol-2-yl)-2,2-dimethylbutanoic acid;
Methyl 2,2-dimethyl-4-(5-(4-(4-phenylpiperidine-1-carboxamido)phenyl)thiazol-2-yl)butanoate;
2,2-Dimethyl-4-(5-(4-(4-phenylpiperidine-1-carboxamido)phenyl)thiazol-2-yl)butanoic acid;
Methyl 2,2-dimethyl-4-(5-(4-(4-phenylpiperidine-1-carboxamido)phenyl)thiazol-2-yl)butanoate;
4-(5-(4-(3-(4-Cyanobenzyl)ureido)phenyl)thiazol-2-yl)-2,2-dimethylbutanoic acid;
Methyl 4-(5-(4-(3-(2-fluorophenyl)thioureido)phenyl)thiazol-2-yl)-2,2-dimethyl butanoate;
4-(5-(4-(3-(2-Fluorophenyl)thioureido)phenyl)thiazol-2-yl)-2,2-dimethylbutanoic acid;
Methyl 4-(5-(4-(3-(2-fluorophenyl)guanidino)phenyl)thiazol-2-yl)-2,2-dimethyl butanoate;
4-(5-(4-(3-(2-Fluorophenyl)guanidino)phenyl)thiazol-2-yl)-2,2-dimethylbutanoic acid;
Methyl 4-(5-(4-(3-(2-fluorophenyl)-2-methylguanidino)phenyl)thiazol-2-yl)-2,2-dimethylbutanoate;
4-(5-(4-(3-(2-Fluorophenyl)-2-methylguanidino)phenyl)thiazol-2-yl)-2,2-dimethylbutanoic acid;
Methyl 4-(5-(4-(2-cyano-3-(2-fluorophenyl)guanidino)phenyl)thiazol-2-yl)-2,2-dimethylbutanoate;
4-(5-(4-(2-Cyano-3-(2-fluorophenyl)guanidino)phenyl)thiazol-2-yl)-2,2-dimethyl butanoic acid;
Methyl 4-(5-(4-(3-(3-(trifluoromethyl)phenyl)ureido)phenyl)-1,3,4-thiadiazol-2-yl)butanoate;
4-(5-(4-(3-(3-(Trifluoromethyl)phenyl)ureido)phenyl)-1,3,4-thiadiazol-2-yl)butanoic acid;
Methyl 4-(5-(4-(3-(2-chlorophenyl)ureido)phenyl)-1,3,4-thiadiazol-2-yl)butanoate;
4-(5-(4-(3-(2-Chlorophenyl)ureido)phenyl)-1,3,4-thiadiazol-2-yl)butanoic acid;
Methyl 4-(5-(4-(3-(p-tolyl)ureido)phenyl)-1,3,4-thiadiazol-2-yl)butanoate;
4-(5-(4-(3-(p-Tolyl)ureido)phenyl)-1,3,4-thiadiazol-2-yl)butanoic acid;
Methyl 4-(5-(4-(3-(2,4-difluorophenyl)ureido)phenyl)-1,3,4-thiadiazol-2-yl)butanoate;
4-(5-(4-(3-(2,4-Difluorophenyl)ureido)phenyl)-1,3,4-thiadiazol-2-yl)butanoic acid;
Methyl 4-(5-(4-(3-(4-chloro-2-phenoxyphenyl)ureido)phenyl)-1,3,4-thiadiazol-2-yl)butanoate;
4-(5-(4-(3-(4-Chloro-2-phenoxyphenyl)ureido)phenyl)-1,3,4-thiadiazol-2-yl)butanoic acid;
Methyl 4-(5-(4-(4-(tert-butyl)benzamido)phenyl)-1,3,4-thiadiazol-2-yl)butanoate;
4-(5-(4-(4-(t-Butyl)benzamido)phenyl)-1,3,4-thiadiazol-2-yl)butanoic acid;
Methyl 4-(5-(4-([1,1′-biphenyl]-4-ylcarboxamido)phenyl)-1,3,4-thiadiazol-2-yl)butanoate;
4-(5-(4-([1,1′-Biphenyl]-4-ylcarboxamido)phenyl)-1,3,4-thiadiazol-2-yl)butanoic acid;
Methyl 4-(5-(4-(4-(trifluoromethoxy)benzamido)phenyl)-1,3,4-thiadiazol-2-yl)butanoate;
4-(5-(4-(4-(trifluoromethoxy)benzamido)phenyl)-1,3,4-thiadiazol-2-yl)butanoic acid;
Methyl 4-(5-(4-(3-(2-chlorophenyl)ureido)phenyl)-1,3,4-oxadiazol-2-yl)butanoate;
4-(5-(4-(3-(2-Chlorophenyl)ureido)phenyl)-1,3,4-oxadiazol-2-yl)butanoic acid;
Methyl 4-(5-(4-(3-(m-tolyl)ureido)phenyl)-1,3,4-oxadiazol-2-yl)butanoate;
4-(5-(4-(3-(m-Tolyl)ureido)phenyl)-1,3,4-oxadiazol-2-yl)butanoic acid;
Methyl 4-(5-(4-(3-(2,4-difluorophenyl)ureido)phenyl)-1,3,4-oxadiazol-2-yl)butanoate;
4-(5-(4-(3-(2,4-Difluorophenyl)ureido)phenyl)-1,3,4-oxadiazol-2-yl)butanoic acid;
Methyl 4-(5-(4-(3-(3-(trifluoromethyl)phenyl)ureido)phenyl)-1,3,4-oxadiazol-2-yl)butanoate;
4-(5-(4-(3-(3-(Trifluoromethyl)phenyl)ureido)phenyl)-1,3,4-oxadiazol-2-yl)butanoic acid;
Ethyl 4-(3-(4-(3-(2-chlorophenyl)ureido)phenyl)-1H-pyrazol-1-yl)cyclohexane carboxylate;
4-(3-(4-(3-(2-Chlorophenyl)ureido)phenyl)-1H-pyrazol-1-yl)cyclohexane carboxylic acid;
Ethyl 4-(3-(4-(3-(2-fluorophenyl)ureido)phenyl)-1H-pyrazol-1-yl)cyclohexane carboxylate;
4-(3-(4-(3-(2-Fluorophenyl)ureido)phenyl)-1H-pyrazol-1-yl)cyclohexane carboxylic acid;
Ethyl 4-(3-(4-(3-(2,4-difluorophenyl)ureido)phenyl)-1H-pyrazol-1-yl)cyclohexane carboxylate;
4-(3-(4-(3-(2,4-Difluorophenyl)ureido)phenyl)-1H-pyrazol-1-yl)cyclohexanecarboxylic acid;
Ethyl 4-(3-(4-(3-(3-(trifluoromethyl)phenyl)ureido)phenyl)-1H-pyrazol-1-yl)cyclohexanecarboxylate;
4-(3-(4-(3-(3-(Trifluoromethyl)phenyl)ureido)phenyl)-1H-pyrazol-1-yl)cyclohexanecarboxylic acid;
Ethyl 4-(3-(4-(3-(m-tolyl)ureido)phenyl)-1H-pyrazol-1-yl)cyclohexane carboxylate;
4-(3-(4-(3-(m-Tolyl)ureido)phenyl)-1H-pyrazol-1-yl)cyclohexanecarboxylic acid;
Methyl 4-(3-(4-(3-(4-chloro-2-phenoxyphenyl)ureido)phenyl)-1,2,4-oxadiazol-5-yl)butanoate;
4-(3-(4-(3-(4-Chloro-2-phenoxyphenyl)ureido)phenyl)-1,2,4-oxadiazol-5-yl)butanoic acid;
Methyl 4-(3-(4-(3-(2,4-difluorophenyl)ureido)phenyl)-1,2,4-oxadiazol-5-yl)butanoate;
4-(3-(4-(3-(2,4-Difluorophenyl)ureido)phenyl)-1,2,4-oxadiazol-5-yl)butanoic acid;
Methyl 4-(3-(4-(3-(2-chlorophenyl)ureido)phenyl)-1,2,4-oxadiazol-5-yl)butanoate;
4-(3-(4-(3-(2-Chlorophenyl)ureido)phenyl)-1,2,4-oxadiazol-5-yl)butanoic acid;
Methyl 4-(3-(4-(3-(4-chloro-2-phenoxyphenyl)ureido)phenyl)-1,2,4-oxadiazol-5-yl)butanoate;
4-(3-(4-(3-(4-Chloro-2-phenoxyphenyl)ureido)phenyl)-1,2,4-oxadiazol-5-yl)butanoic acid;
Methyl 4-(3-(4-(3-(2,4-difluorophenyl)ureido)phenyl)-1,2,4-oxadiazol-5-yl)butanoate;
4-(3-(4-(3-(2,4-Difluorophenyl)ureido)phenyl)-1,2,4-oxadiazol-5-yl)butanoic acid;
Methyl 4-(3-(4-(3-(2-chlorophenyl)ureido)phenyl)-1,2,4-oxadiazol-5-yl)butanoate;
4-(3-(4-(3-(2-Chlorophenyl)ureido)phenyl)-1,2,4-oxadiazol-5-yl)butanoic acid;
Methyl 4-(3-(4-(4-fluorobenzamido)phenyl)-1,2,4-oxadiazol-5-yl)-2,2-dimethyl butanoate;
4-(3-(4-(4-Fluorobenzamido)phenyl)-1,2,4-oxadiazol-5-yl)-2,2-dimethylbutanoic acid;
Methyl 4-(3-(4-([1,1′-biphenyl]-4-ylcarboxamido)phenyl)-1,2,4-oxadiazol-5-yl)-2,2-dimethylbutanoate;
4-(3-(4-([1,1′-Biphenyl]-4-ylcarboxamido)phenyl)-1,2,4-oxadiazol-5-yl)-2,2-dimethylbutanoic acid;
t-Butyl 2-(4-(5-(4-(3-(2-chlorophenyl)ureido)phenyl)thiazol-2-yl)cyclohexyl)acetate;
2-(4-(5-(4-(3-(2-Chlorophenyl)ureido)phenyl)thiazol-2-yl)cyclohexyl)acetic acid;
t-Butyl 2-(4-(5-(4-(3-(2-fluorophenyl)ureido)phenyl)thiazol-2-yl)cyclohexyl)acetate;
2-(4-(5-(4-(3-(2-Fluorophenyl)ureido)phenyl)thiazol-2-yl)cyclohexyl)acetic acid;
Ethyl 2-(4-(5-(4-(3-(3,5-difluorophenyl)ureido)phenyl)thiazol-2-yl)cyclohexyl)acetate;
2-(4-(5-(4-(3-(3,5-Difluorophenyl)ureido)phenyl)thiazol-2-yl)cyclohexyl)acetic acid;
Ethyl 2-(4-(5-(4-(3-(2,4,5-trifluorophenyl)ureido)phenyl)thiazol-2-yl)cyclohexyl)acetate;
2-(4-(5-(4-(3-(2,4,5-Trifluorophenyl)ureido)phenyl)thiazol-2-yl)cyclohexyl)acetic acid;
Ethyl 2-(4-(5-(4-(3-(2,4,6-trifluorophenyl)ureido)phenyl)thiazol-2-yl)cyclohexyl)acetate;
2-(4-(5-(4-(3-(2,4,6-Trifluorophenyl)ureido)phenyl)thiazol-2-yl)cyclohexyl)acetic acid;
Ethyl 2-(4-(5-(4-(3-(2,4-difluorophenyl)ureido)phenyl)thiazol-2-yl)cyclohexyl)acetate;
2-(4-(5-(4-(3-(2,4-Difluorophenyl)ureido)phenyl)thiazol-2-yl)cyclohexyl)acetic acid;
Ethyl 2-(4-(5-(4-(2,4-dichlorobenzamido)phenyl)thiazol-2-yl)cyclohexyl)acetate;
2-(4-(5-(4-(2,4-Dichlorobenzamido)phenyl)thiazol-2-yl)cyclohexyl)acetic acid;
Ethyl 2-(4-(5-(4-(2-fluoro-6-(trifluoromethyl)benzamido)phenyl)thiazol-2-yl)cyclohexyl)acetate;
2-(4-(5-(4-(2-Fluoro-6-(trifluoromethyl)benzamido)phenyl)thiazol-2-yl)cyclohexyl)acetic acid;
Ethyl 2-(4-(5-(4-(3-(3,4,5-trifluorophenyl)ureido)phenyl)thiazol-2-yl)cyclohexyl)acetate;
2-(4-(5-(4-(3-(3,4,5-Trifluorophenyl)ureido)phenyl)thiazol-2-yl)cyclohexyl)acetic acid;
Ethyl 2-(4-(5-(4-(2-phenyl-5-(trifluoromethyl)oxazole-4-carboxamido)phenyl)thiazol-2-yl)cyclohexyl)acetate;
2-(4-(5-(4-(2-Phenyl-5-(trifluoromethyl)oxazole-4-carboxamido)phenyl)thiazol-2-yl)cyclohexyl)acetic acid;
Ethyl 2-(4-(5-(4-(5-methyl-2-phenyloxazole-4-carboxamido)phenyl)thiazol-2-yl)cyclohexyl)acetate;
2-(4-(5-(4-(5-Methyl-2-phenyloxazole-4-carboxamido)phenyl)thiazol-2-yl)cyclohexyl)acetic acid;
Ethyl 2-(4-(5-(4-(3-(2-fluorophenyl)thioureido)phenyl)thiazol-2-yl)cyclohexyl)acetate;
2-(4-(5-(4-(3-(2-Fluorophenyl)thioureido)phenyl)thiazol-2-yl)cyclohexyl)acetic acid;
Ethyl 2-(4-(5-(4-(3-(2-fluorophenyl)guanidino)phenyl)thiazol-2-yl)cyclohexyl)acetate;
4-(2-(4-((5-Methyl-1,3,4-oxadiazol-2-yl)methyl)cyclohexyl)thiazol-5-yl)aniline;
1-(2,4-Difluorophenyl)-3-(4-(2-(4-((5-methyl-1,3,4-oxadiazol-2-yl)methyl)cyclohexyl)thiazol-5-yl)phenyl)urea;
1-(2-Chlorophenyl)-3-(4-(2-(4-((5-methyl-1,3,4-oxadiazol-2-yl)methyl)cyclohexyl)thiazol-5-yl)phenyl)urea;
1-(3,5-Difluorophenyl)-3-(4-(2-(4-((5-methyl-1,3,4-oxadiazol-2-yl)methyl)cyclohexyl)thiazol-5-yl)phenyl)urea;
1-(4-(2-(4-((5-Methyl-1,3,4-oxadiazol-2-yl)methyl)cyclohexyl)thiazol-5-yl)phenyl)-3-(2,4,5-trifluorophenyl)urea;
1-(4-(2-(4-((5-Methyl-1,3,4-oxadiazol-2-yl)methyl)cyclohexyl)thiazol-5-yl)phenyl)-3-(2,4,6-trifluorophenyl)urea;
1-(4-(2-(4-((5-Methyl-1,3,4-oxadiazol-2-yl)methyl)cyclohexyl)thiazol-5-yl)phenyl)-3-phenylurea;
2,6-Difluoro-N-(4-(2-(4-((5-methyl-1,3,4-oxadiazol-2-yl)methyl)cyclohexyl)thiazol-5-yl)phenyl)benzamide;
4-(2-(4-((3-Methyl-1,2,4-oxadiazol-5-yl)methyl)cyclohexyl)thiazol-5-yl)aniline;
1-(2-Chlorophenyl)-3-(4-(2-(4-((3-methyl-1,2,4-oxadiazol-5-yl)methyl)cyclohexyl)thiazol-5-yl)phenyl)urea;
1-(2-Fluorophenyl)-3-(4-(2-(4-((3-methyl-1,2,4-oxadiazol-5-yl)methyl)cyclohexyl)thiazol-5-yl)phenyl)urea;
1-(3,5-Difluorophenyl)-3-(4-(2-(4-((3-methyl-1,2,4-oxadiazol-5-yl)methyl)cyclohexyl)thiazol-5-yl)phenyl)urea;
1-(4-(2-(4-((3-Methyl-1,2,4-oxadiazol-5-yl)methyl)cyclohexyl)thiazol-5-yl)phenyl)-3-(2,4,5-trifluorophenyl)urea;
1-(2,4-Difluorophenyl)-3-(4-(2-(4-((3-methyl-1,2,4-oxadiazol-5-yl)methyl)cyclohexyl)thiazol-5-yl)phenyl)urea;
1-(4-(2-(4-((3-Methyl-1,2,4-oxadiazol-5-yl)methyl)cyclohexyl)thiazol-5-yl)phenyl)-3-phenylurea;
2,6-Difluoro-N-(4-(2-(4-((3-methyl-1,2,4-oxadiazol-5-yl)methyl)cyclohexyl)thiazol-5-yl)phenyl)benzamide;
2-Chloro-N-(4-(2-(4-((3-methyl-1,2,4-oxadiazol-5-yl)methyl)cyclohexyl)thiazol-5-yl)phenyl)benzamide;
3,5-Difluoro-N-(4-(2-(4-((3-methyl-1,2,4-oxadiazol-5-yl)methyl)cyclohexyl)thiazol-5-yl)phenyl)benzamide;
N-Acetyl-2-(4-(5-(4-aminophenyl)thiazol-2-yl)cyclohexyl)acetamide;
N-Acetyl-2-(4-(5-(4-(3-(2-chlorophenyl)ureido)phenyl)thiazol-2-yl)cyclohexyl)acetamide;
N-Acetyl-2-(4-(5-(4-(3-(2,4-difluorophenyl)ureido)phenyl)thiazol-2-yl)cyclohexyl)acetamide;
N-Acetyl-2-(4-(5-(4-(3-(2,4,5-trifluorophenyl)ureido)phenyl)thiazol-2-yl)cyclohexyl)acetamide;
N-(4-(2-(4-(2-Acetamido-2-oxoethyl)cyclohexyl)thiazol-5-yl)phenyl)-2,6-difluoro benzamide;
1-(2-Chlorophenyl)-3-(4-(2-(4-(2-hydroxypropan-2-yl)cyclohexyl)thiazol-5-yl)phenyl)urea;
1-(3,5-Difluorophenyl)-3-(4-(2-(4-(2-hydroxypropan-2-yl)cyclohexyl)thiazol-5-yl)phenyl)urea;
1-(2,4-Difluorophenyl)-3-(4-(2-(4-(2-hydroxypropan-2-yl)cyclohexyl)thiazol-5-yl) phenyl)urea;
1-(2,4-Difluorophenyl)-3-(4-(2-(4-(2-hydroxy-2-methylpropyl)cyclohexyl)thiazol-5-yl)phenyl)urea;
1-(3,5-Difluorophenyl)-3-(4-(2-(4-(2-hydroxy-2-methylpropyl)cyclohexyl)thiazol-5-yl)phenyl)urea;
1-(4-(2-(4-(2-Hydroxy-2-methylpropyl)cyclohexyl)thiazol-5-yl)phenyl)-3-(2,4,5-trifluorophenyl)urea;
1-(3,5-Difluorophenyl)-3-(4-(2-(4-(2-hydrazinyl-2-oxoethyl)cyclohexyl)thiazol-5-yl)phenyl)urea;
4-(2-(4-((5-Methyl-1,3,4-thiadiazol-2-yl)methyl)cyclohexyl)thiazol-5-yl)aniline;
1-(4-(2-(4-((5-Methyl-1,3,4-thiadiazol-2-yl)methyl)cyclohexyl)thiazol-5-yl)phenyl)-3-(2,4,5-trifluorophenyl)urea;
Ethyl 2-(4-(4-(4-(3-(2-fluorophenyl)ureido)phenyl)thiazol-2-yl)piperidin-1-yl)acetate;
2-(4-(4-(4-(3-(2-Fluorophenyl)ureido)phenyl)thiazol-2-yl)piperidin-1-yl)acetic acid;
Ethyl 2-(4-(4-(4-(3-(2-chlorophenyl)ureido)phenyl)thiazol-2-yl)piperidin-1-yl)acetate;
2-(4-(4-(4-(3-(2-Chlorophenyl)ureido)phenyl)thiazol-2-yl)piperidin-1-yl)acetic acid;
Ethyl 2-(4-(5-(4-(3-(2-chlorophenyl)ureido)phenyl)thiazol-2-yl)piperidin-1-yl)acetate;
2-(4-(5-(4-(3-(2-Chlorophenyl)ureido)phenyl)thiazol-2-yl)piperidin-1-yl)acetic acid;
Ethyl 2-(4-(5-(4-(3-(2-fluorophenyl)ureido)phenyl)thiazol-2-yl)piperidin-1-yl)acetate;
2-(4-(5-(4-(3-(2-Fluorophenyl)ureido)phenyl)thiazol-2-yl)piperidin-1-yl)acetic acid;
Ethyl 2-(4-(5-(4-(3-(2,4-difluorophenyl)ureido)phenyl)thiazol-2-yl)piperidin-1-yl)acetate;
2-(4-(5-(4-(3-(2,4-Difluorophenyl)ureido)phenyl)thiazol-2-yl)piperidin-1-yl)acetic acid;
Ethyl 2-(4-(5-(4-(3-(2,4,5-trifluorophenyl)ureido)phenyl)thiazol-2-yl)piperidin-1-yl)acetate;
2-(4-(5-(4-(3-(2,4,5-Trifluorophenyl)ureido)phenyl)thiazol-2-yl)piperidin-1-yl)acetic acid;
Ethyl 2-(4-(5-(4-(3-(2-(trifluoromethyl)phenyl)ureido)phenyl)thiazol-2-yl)piperidin-1-yl)acetate;
2-(4-(5-(4-(3-(2-(Trifluoromethyl)phenyl)ureido)phenyl)thiazol-2-yl)piperidin-1-yl)acetic acid;
Ethyl 2-(4-(5-(4-(3-(2,3,4-trifluorophenyl)ureido)phenyl)thiazol-2-yl)piperidin-1-yl)acetate;
2-(4-(5-(4-(3-(2,3,4-Trifluorophenyl)ureido)phenyl)thiazol-2-yl)piperidin-1-yl)acetic acid;
Ethyl 2-(4-(5-(4-(3-(2,4,6-trifluorophenyl)ureido)phenyl)thiazol-2-yl)piperidin-1-yl)acetate;
2-(4-(5-(4-(3-(2,4,6-Trifluorophenyl)ureido)phenyl)thiazol-2-yl)piperidin-1-yl)acetic acid;
Ethyl 2-methyl-2-(4-(5-(4-(3-(2,4,5-trifluorophenyl)ureido)phenyl)thiazol-2-yl)piperidin-1-yl)propanoate;
Ethyl 2-(4-(5-(4-(3-(2-fluorophenyl)ureido)phenyl)thiazol-2-yl)piperidin-1-yl)-2-methylpropanoate;
Ethyl 2-(4-(5-(4-(3-(2-chlorophenyl)ureido)phenyl)thiazol-2-yl)piperidin-1-yl)-2-methylpropanoate;
Ethyl 2-(4-(5-(4-(3-(2,4-difluorophenyl)ureido)phenyl)thiazol-2-yl)piperidin-1-yl)-2-methylpropanoate;
t-Butyl 2-(4-(5-(4-(3-(2,4,5-trifluorophenyl)ureido)phenyl)thiazol-2-yl)piperidin-1-yl)propanoate;
2-(4-(5-(4-(3-(2,4,5-Trifluorophenyl)ureido)phenyl)thiazol-2-yl)piperidin-1-yl)propanoic acid;
t-Butyl 2-(4-(5-(4-(3-(2-fluorophenyl)ureido)phenyl)thiazol-2-yl)piperidin-1-yl)propanoate;
2-(4-(5-(4-(3-(2-Fluorophenyl)ureido)phenyl)thiazol-2-yl)piperidin-1-yl)propanoic acid;
t-Butyl 2-(4-(5-(4-(3-(2-chlorophenyl)ureido)phenyl)thiazol-2-yl)piperidin-1-yl)propanoate;
2-(4-(5-(4-(3-(2-Chlorophenyl)ureido)phenyl)thiazol-2-yl)piperidin-1-yl)propanoic acid;
t-Butyl 2-(4-(5-(4-(3-(2,4-difluorophenyl)ureido)phenyl)thiazol-2-yl)piperidin-1-yl)propanoate;
2-(4-(5-(4-(3-(2,4-Difluorophenyl)ureido)phenyl)thiazol-2-yl)piperidin-1-yl)propanoic acid;
t-Butyl 2-(4-(5-(4-(3-(2,4,6-trifluorophenyl)ureido)phenyl)thiazol-2-yl)piperidin-1-yl)propanoate;
2-(4-(5-(4-(3-(2,4,6-Trifluorophenyl)ureido)phenyl)thiazol-2-yl)piperidin-1-yl) propanoic acid;
t-Butyl 2-methyl-2-(4-(5-(4-(3-(2,4,5-trifluorophenyl)ureido)phenyl)thiazol-2-yl)piperidin-1-yl)propanoate;
2-Methyl-2-(4-(5-(4-(3-(2,4,5-trifluorophenyl)ureido)phenyl)thiazol-2-yl)piperidin-1-yl)propanoic acid;
t-Butyl 2-(4-(5-(4-(3-(2-fluorophenyl)ureido)phenyl)thiazol-2-yl)piperidin-1-yl)-2-methylpropanoate;
2-(4-(5-(4-(3-(2-Fluorophenyl)ureido)phenyl)thiazol-2-yl)piperidin-1-yl)-2-methylpropanoic acid;
t-Butyl 2-(4-(5-(4-(3-(2-chlorophenyl)ureido)phenyl)thiazol-2-yl)piperidin-1-yl)-2-methylpropanoate;
2-(4-(5-(4-(3-(2-Chlorophenyl)ureido)phenyl)thiazol-2-yl)piperidin-1-yl)-2-methyl propanoic acid;
t-Butyl 2-(4-(5-(4-(3-(2,4-difluorophenyl)ureido)phenyl)thiazol-2-yl)piperidin-1-yl)-2-methylpropanoate;
2-(4-(5-(4-(3-(2,4-Difluorophenyl)ureido)phenyl)thiazol-2-yl)piperidin-1-yl)-2-methylpropanoic acid;
t-Butyl 2-methyl-2-(4-(5-(4-(3-(2,4,6-trifluorophenyl)ureido)phenyl)thiazol-2-yl)piperidin-1-yl)propanoate;
2-Methyl-2-(4-(5-(4-(3-(2,4,6-trifluorophenyl)ureido)phenyl)thiazol-2-yl)piperidin-1-yl)propanoic acid;
t-Butyl 4-(5-(4-(3-(2-chlorophenyl)ureido)phenyl)thiazol-2-yl)piperidine-1-carboxylate;
1-(2-Chlorophenyl)-3-(4-(2-(piperidin-4-yl)thiazol-5-yl)phenyl)urea hydrochloride;
t-Butyl 4-(5-(4-(3-(2-fluorophenyl)ureido)phenyl)thiazol-2-yl)piperidine-1-carboxylate;
1-(2-Fluorophenyl)-3-(4-(2-(piperidin-4-yl)thiazol-5-yl)phenyl)urea hydrochloride;
t-Butyl 4-(5-(4-(3-(2,4-difluorophenyl)ureido)phenyl)thiazol-2-yl)piperidine-1-carboxylate;
1-(2,4-Difluorophenyl)-3-(4-(2-(piperidin-4-yl)thiazol-5-yl)phenyl)urea hydrochloride;
t-Butyl 4-(5-(4-(3-(2,4,6-trifluorophenyl)ureido)phenyl)thiazol-2-yl)piperidine-1-carboxylate;
1-(4-(2-(Piperidin-4-yl)thiazol-5-yl)phenyl)-3-(2,4,5-trifluorophenyl)urea hydrochloride;
1-(2-Fluorophenyl)-3-(4-(2-(1-((trifluoromethyl)sulfonyl)piperidin-4-yl)thiazol-5-yl)phenyl)urea;
1-(2-Chlorophenyl)-3-(4-(2-(1-((trifluoromethyl)sulfonyl)piperidin-4-yl)thiazol-5-yl)phenyl)urea;
1-(2,4-Difluorophenyl)-3-(4-(2-(1-((trifluoromethyl)sulfonyl)piperidin-4-yl)thiazol-5-yl)phenyl)urea;
1-(4-(2-(1-(Trifluoromethyl)sulfonyl)piperidin-4-yl)thiazol-5-yl)phenyl)-3-(2,4,6-trifluorophenyl)urea;
1-(4-(2-(1-((Trifluoromethyl)sulfonyl)piperidin-4-yl)thiazol-5-yl)phenyl)-3-(2,4,5-trifluorophenyl)urea;
1-(2-Chlorophenyl)-3-(4-(2-(1-(methylsulfonyl)piperidin-4-yl)thiazol-5-yl)phenyl)urea;
1-(2-Fluorophenyl)-3-(4-(2-(1-(methylsulfonyl)piperidin-4-yl)thiazol-5-yl)phenyl)urea;
1-(2,4-Difluorophenyl)-3-(4-(2-(1-(methylsulfonyl)piperidin-4-yl)thiazol-5-yl)phenyl)urea;
1-(4-(2-(1-(Methylsulfonyl)piperidin-4-yl)thiazol-5-yl)phenyl)-3-(2,4,6-trifluoro phenyl)urea;
1-(4-(2-(1-(Methylsulfonyl)piperidin-4-yl)thiazol-5-yl)phenyl)-3-(2,4,5-trifluoro phenyl)urea;
Methyl 3-(5-(4-(3-(2-chlorophenyl)ureido)phenyl)thiazol-2-yl)adamantane-1-carboxylate;
3-(5-(4-(3-(2-Chlorophenyl)ureido)phenyl)thiazol-2-yl)adamantane-1-carboxylic acid;
Methyl 3-(5-(4-(3-(2-fluorophenyl)ureido)phenyl)thiazol-2-yl)adamantane-1-carboxylate;
3-(5-(4-(3-(2-Fluorophenyl)ureido)phenyl)thiazol-2-yl)adamantane-1-carboxylic acid;
Methyl 3-(5-(4-(3-(2,4-difluorophenyl)ureido)phenyl)thiazol-2-yl)adamantine-1-carboxylate;
3-(5-(4-(3-(2,4-Difluorophenyl)ureido)phenyl)thiazol-2-yl)adamantane-1-carboxylic acid;
Methyl 3-(5-(4-(3-(2,6-difluorophenyl)ureido)phenyl)thiazol-2-yl)adamantine-1-carboxylate;
3-(5-(4-(3-(2,6-Difluorophenyl)ureido)phenyl)thiazol-2-yl)adamantane-1-carboxylic acid;
Methyl 3-(5-(4-(3-(2,4,5-trifluorophenyl)ureido)phenyl)thiazol-2-yl)adamantane-1-carboxylate;
3-(5-(4-(3-(2,4,5-Trifluorophenyl)ureido)phenyl)thiazol-2-yl)adamantane-1-carboxylic acid;
Methyl 3-(5-(4-(3-(2,3,4-trifluorophenyl)ureido)phenyl)thiazol-2-yl)adamantane-1-carboxylate;
3-(5-(4-(3-(2,3,4-Trifluorophenyl)ureido)phenyl)thiazol-2-yl)adamantane-1-carboxylic acid;
Methyl 3-(5-(4-(3-(3,5-difluorophenyl)ureido)phenyl)thiazol-2-yl)adamantane-1-carboxylate;
3-(5-(4-(3-(3,5-Difluorophenyl)ureido)phenyl)thiazol-2-yl)adamantane-1-carboxylic acid;
Methyl 3-(5-(4-(3-(3-(trifluoromethyl)phenyl)ureido)phenyl)thiazol-2-yl)adamantane-1-carboxylate;
3-(5-(4-(3-(3-(Trifluoromethyl)phenyl)ureido)phenyl)thiazol-2-yl)adamantane-1-carboxylic acid;
N-(2-(5-(4-(3-(2-Chlorophenyl)ureido)phenyl)thiazol-2-yl)ethyl)-1,1,1-trifluoro methanesulfonamide;
1,1,1-Trifluoro-N-(2-(5-(4-(3-(2-fluorophenyl)ureido)phenyl)thiazol-2-yl)ethyl)methanesulfonamide;
N-(2-(5-(4-(3-(3,5-Difluorophenyl)ureido)phenyl)thiazol-2-yl)ethyl)-1,1,1-trifluoromethanesulfonamide;
1,1,1-Trifluoro-N-(2-(5-(4-(3-(2,4,5-trifluorophenyl)ureido)phenyl)thiazol-2-yl)ethyl)methanesulfonamide;
1,1,1-Trifluoro-N-(2-(5-(4-(3-(2,4,6-trifluorophenyl)ureido)phenyl)thiazol-2-yl)ethyl)methanesulfonamide;
1,1,1-Trifluoro-N-(2-(5-(4-(3-(4-(trifluoromethyl)phenyl)ureido)phenyl)thiazol-2-yl)ethyl)methanesulfonamide;
1,1,1-Trifluoro-N-(2-(5-(4-(3-phenylureido)phenyl)thiazol-2-yl)ethyl)methane sulfonamide;
N-(2-(5-(4-(3-Cyclohexylureido)phenyl)thiazol-2-yl)ethyl)-1,1,1-trifluoro methanesulfonamide;
2-Chloro-N-(4-(2-(2-(trifluoromethylsulfonamido)ethyl)thiazol-5-yl)phenyl)benzamide;
N-(4-(2-(2-(Trifluoromethylsulfonamido)ethyl)thiazol-5-yl)phenyl)cyclohexane carboxamide;
4-(Trifluoromethyl)-N-(4-(2-(2-(trifluoromethylsulfonamido)ethyl)thiazol-5-yl)phenyl)benzamide;
N-(4-(2-(2-(Trifluoromethylsulfonamido)ethyl)thiazol-5-yl)phenyl)benzamide;
2-Phenyl-5-(trifluoromethyl)-N-(4-(2-(2-(trifluoromethylsulfonamido)ethyl)thiazol-5-yl)phenyl)oxazole-4-carboxamide;
1,1,1-Trifluoro-N-(2-(5-(4-(3-(2-fluorophenyl)thioureido)phenyl)thiazol-2-yl)ethyl)methanesulfonamide;
1,1,1-Trifluoro-N-(2-(5-(4-(3-(2-fluorophenyl)guanidino)phenyl)thiazol-2-yl)ethyl)methanesulfonamide;
1,1,1-Trifluoro-N-(2-(5-(4-(3-(2-fluorophenyl)-2-methylguanidino)phenyl)thiazol-2-yl)ethyl)methanesulfonamide;
N-(2-(5-(4-(2-Cyano-3-(2-fluorophenyl)guanidino)phenyl)thiazol-2-yl)ethyl)-1,1,1-trifluoromethanesulfonamide;
N-((5-(4-(3-(2-Chlorophenyl)ureido)phenyl)thiazol-2-yl)methyl)-1,1,1-trifluoro methanesulfonamide;
1,1,1-Trifluoro-N-((5-(4-(3-(2-fluorophenyl)ureido)phenyl)thiazol-2-yl)methyl)methanesulfonamide;
N-((5-(4-(3-(3,5-Difluorophenyl)ureido)phenyl)thiazol-2-yl)methyl)-1,1,1-trifluoromethanesulfonamide;
1,1,1-Trifluoro-N-((5-(4-(3-(2,4,5-trifluorophenyl)ureido)phenyl)thiazol-2-yl)methyl)methanesulfonamide;
1,1,1-Trifluoro-N-((5-(4-(3-(2,4,6-trifluorophenyl)ureido)phenyl)thiazol-2-yl)methyl)methanesulfonamide;
N-((5-(4-(3-Cyclohexylureido)phenyl)thiazol-2-yl)methyl)-1,1,1-trifluoro methanesulfonamide;
1,1,1-Trifluoro-N-((5-(4-(3-(4-(trifluoromethyl)phenyl)ureido)phenyl)thiazol-2-yl)methyl)methanesulfonamide;
1,1,1-Trifluoro-N-((5-(4-(3-phenylureido)phenyl)thiazol-2-yl)methyl)methane sulfonamide;
2-Chloro-N-(4-(2-((trifluoromethylsulfonamido)methyl)thiazol-5-yl)phenyl)benzamide;
4-(Trifluoromethyl)-N-(4-(2-((trifluoromethylsulfonamido)methyl)thiazol-5-yl)phenyl)benzamide;
N-(4-(2-((Trifluoromethylsulfonamido)methyl)thiazol-5-yl)phenyl)benzene sulfonamide;
4-(Trifluoromethyl)-N-(4-(2-((trifluoromethylsulfonamido)methyl)thiazol-5-yl)phenyl)benzenesulfonamide;
N-(4-(2-((Trifluoromethylsulfonamido)methyl)thiazol-5-yl)phenyl)cyclohexane sulfonamide;
2,4-Difluoro-N-(4-(2-((trifluoromethylsulfonamido)methyl)thiazol-5-yl)phenyl)benzenesulfonamide;
N-(2-(5-(4-(3-(2-Chlorophenyl)ureido)phenyl)thiazol-2-yl)propan-2-yl)-1,1,1-trifluoromethanesulfonamide;
1,1,1-Trifluoro-N-(2-(5-(4-(3-(2-fluorophenyl)ureido)phenyl)thiazol-2-yl)propan-2-yl)methanesulfonamide;
N-(2-(5-(4-(3-(3,5-Difluorophenyl)ureido)phenyl)thiazol-2-yl)propan-2-yl)-1,1,1-trifluoromethanesulfonamide;
1,1,1-Trifluoro-N-(2-(5-(4-(3-(2,4,5-trifluorophenyl)ureido)phenyl)thiazol-2-yl)propan-2-yl)methanesulfonamide;
1,1,1-Trifluoro-N-(2-(5-(4-(3-(2,4,6-trifluorophenyl)ureido)phenyl)thiazol-2-yl)propan-2-yl)methanesulfonamide;
N-(2-(5-(4-(3-Cyclohexylureido)phenyl)thiazol-2-yl)propan-2-yl)-1,1,1-trifluoromethanesulfonamide;
N-(4-(2-(2-(Trifluoromethylsulfonamido)propan-2-yl)thiazol-5-yl)phenyl)benzenesulfonamide;
t-Butyl (2-(5-(4-(3-(2-chlorophenyl)ureido)phenyl)thiazol-2-yl)ethyl)carbamate;
t-Butyl (2-(5-(4-(3-(3,5-difluorophenyl)ureido)phenyl)thiazol-2-yl)ethyl)carbamate:
t-Butyl (2-(5-(4-(3-(2,4,5-trifluorophenyl)ureido)phenyl)thiazol-2-yl)ethyl)carbamate;
1-(4-(2-(2-Aminoethyl)thiazol-5-yl)phenyl)-3-(2-chlorophenyl)urea hydrochloride;
1-(4-(2-(2-Aminoethyl)thiazol-5-yl)phenyl)-3-(3,5-difluorophenyl)urea hydrochloride;
1-(4-(2-(2-Aminoethyl)thiazol-5-yl)phenyl)-3-(2,4,5-trifluorophenyl)urea hydrochloride;
4-(5-(4-(3-(2-Chlorophenyl)ureido)phenyl)thiazol-2-yl)-2,2-dimethyl-N-((trifluoromethyl)sulfonyl)butanamide;
4-(5-(4-(3-(2-Fluorophenyl)ureido)phenyl)thiazol-2-yl)-2,2-dimethyl-N-((trifluoromethyl)sulfonyl)butanamide;
4-(5-(4-(3-(3,5-Difluorophenyl)ureido)phenyl)thiazol-2-yl)-2,2-dimethyl-N-((trifluoromethyl)sulfonyl)butanamide;
2,2-Dimethyl-N-((trifluoromethyl)sulfonyl)-4-(5-(4-(3-(2,4,5-trifluorophenyl)ureido) phenyl)thiazol-2-yl)butanamide;
Methyl 4-(5-(4-(3-(2,4,5-trifluorophenyl)ureido)phenyl)thiazol-2-yl)cyclohexane carboxylate;
4-(5-(4-(3-(2,4,5-Trifluorophenyl)ureido)phenyl)thiazol-2-yl)cyclohexanecarboxylic acid;
1-(4-(2-(4-(2-Hydroxypropan-2-yl)cyclohexyl)thiazol-5-yl)phenyl)-3-(2,4,5-trifluorophenyl)urea;
1-(4-(2-(4-(2-Aminopropan-2-yl)cyclohexyl)thiazol-5-yl)phenyl)-3-(2,4,5-trifluoro phenyl)urea;
1-(4-(2-(4-(2-Aminopropan-2-yl)cyclohexyl)thiazol-5-yl)phenyl)-3-(2,4-difluoro phenyl)urea; and
1-(4-(2-(4-(2-Amino-2-methylpropyl)cyclohexyl)thiazol-5-yl)phenyl)-3-(2,4,5-trifluorophenyl)urea;
or a stereoisomer, tautomer, pharmaceutically acceptable salt or N-oxide thereof.

95. A pharmaceutical composition comprising a compound according to claim 68, or a stereoisomer, a tautomer, a pharmaceutically acceptable salt or N-oxide thereof, and a pharmaceutically acceptable excipient or a carrier.

96. A method of treatment of a diacylglycerol acyltransferase 1 (DGAT1) mediated disease or disorder comprising administering to a subject in need thereof, a therapeutically effective amount of a compound according to claim 68, or a stereoisomer, tautomer, pharmaceutically acceptable salt or N-oxide thereof.

97. The method according to claim 96, wherein the DGAT1 mediated disease or disorder is selected from obesity, diabetes, impaired glucose tolerance, diabetic neuropathy, diabetic nephropathy, diabetic retinopathy, anorexia nervosa, bulimia, cachexia, syndrome X, insulin resistance, hypoglycemia, hyperglycemia, hyperuricemia, hyperinsulinemia, hypercholesterolemia, hyperlipidemia, dyslipidemia, mixed dyslipidemia, hypertriglyceridemia, pancreatitis, metabolic acidosis, ketosis, steatosis, dysmetabolic syndrome and nonalcoholic fatty liver disease, skin disorders, acne, atherosclerosis, arteriosclerosis, acute heart failure, congestive heart failure, coronary artery disease, cardiomyopathy, myocardial ischaemia, myocardial infarction, angina pectoris, hypertension, hypotension, stroke, ischemia, ischemic reperfusion injury, aneurysm, restenosis, peripheral vascular disease and vascular stenosis, infertility, polycystic ovary syndrome or Hepatitis C infection.

98. The method according to claim 97, wherein the DGAT1 mediated disease or disorder is selected from impaired glucose tolerance, diabetes, insulin resistance, diabetic neuropathy, diabetic nephropathy, diabetic retinopathy, hypercholesterolemia, hypertriglyceridemia, hyperlipidemia or obesity.

99. The method according to claim 98, wherein the DGAT1 mediated disease or disorder is obesity.

100. A compound of formula D: 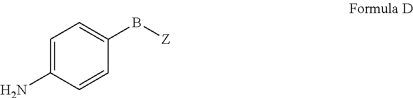
wherein B and Z are as defined in claim 68; for use as an intermediate in the preparation of the compound according to claim 68.

101. A process for the preparation of a compound of formula 1a as defined in claim 69: 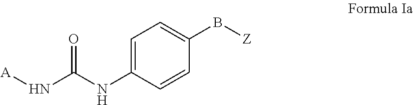
wherein A, B and Z are as defined in claim 68;
comprising the steps of:
step a) treating the compound of formula D: 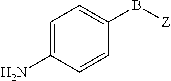
wherein B and Z are as defined in claim 68;
with a compound of formula 8 (1):
A-N═C═O 8 (i)
wherein A is as defined in claim 68;
in a solvent selected from tetrahydrofuran (THF) or dichloromethane at room temperature for 2-16 h;
or treating the compound of formula D: 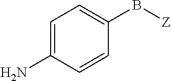
with the compound of formula 8 (ii):
A-NH₂ 8 (ii)
wherein A is as defined in claim 68;
in the presence of carbonyl diimidazole as the coupling agent in THF as the solvent at room temperature for 24 h; and
step b) hydrolysis of the compound of formula 1a;
wherein Z is: 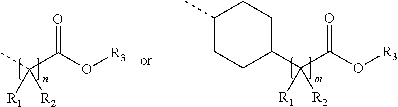
R₁, R₂ and n are as defined in claim 68; and
R₃ is (C₁-C₁₂)-alkyl;
by reaction with aqueous lithium hydroxide (LiOH) in a solvent selected from THF or methanol or a mixture thereof, at room temperature for 2-16 h into the corresponding carboxylic acid, the compound of formula 1a (wherein R₃ is H); and conversion of the carboxylic acid obtained into its corresponding pharmaceutically acceptable salt.

102. A process for the preparation of a compound of formula 1b as defined in claim 75: 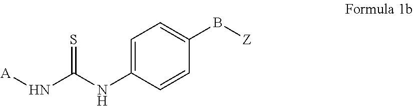
wherein A, B and Z are as defined in claim 68;
comprising the steps of:
step a) treating the compound of formula D: 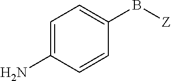
wherein B and Z are as defined in claim 68;
with a compound of formula 8 (iii):
A-N═C═S 8 (iii)
wherein A is as defined in claim 68;
in a solvent selected from THF or dichloromethane at room temperature for 2-16 h; and
step b) hydrolysis of the compound of formula 1b;
wherein Z is: 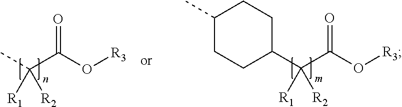
R₁, R₂ and n are as defined in claim 68; and
R₃ is (C₁-C₁₂)-alkyl;
by reaction with aqueous LiOH in a solvent selected from THF or methanol or a mixture thereof, at room temperature for 2-16 h into the corresponding carboxylic acid, the compound of formula 1b (wherein R₃ is H); and conversion of the said carboxylic acid obtained into its corresponding pharmaceutically acceptable salt.

103. A process for the preparation of a compound of formula 1c as defined in claim 81: 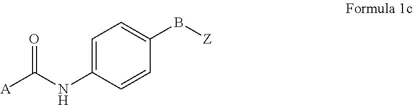
wherein A, B and Z are as defined in claim 68;
comprising the steps of:
step a) treating the compound of formula D: 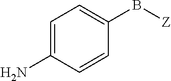
wherein B and Z are as defined in claim 68;
with a compound of formula 8 (iv):
A-C(O)—Cl 8 (iv)
wherein A is as defined in claim 68;
in a solvent selected from dichloromethane or chloroform in pyridine as the base at room temperature for 1-2 h;
or by reacting compound of formula D: 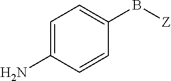
with a compound of formula 8 (v):
A-COOR₃ 8(v)
wherein A and R₃ are as defined in claim 68; in toluene as the solvent and
trimethylaluminium as the coupling agent; and
step b) hydrolysis of the compound of formula 1c;
wherein Z is: 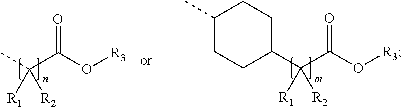
R₁, R₂ and n are as defined in claim 68; and
R₃ is (C₁-C₁₂)-alkyl;
by reaction with aqueous LiOH in a solvent selected from THF or methanol or a mixture thereof, at room temperature for 2-16 h into the corresponding carboxylic acid, the compound of formula 1c (wherein R₃ is H); and conversion of the said carboxylic acid obtained into its corresponding pharmaceutically acceptable salt.

104. A process for the preparation of a compound of formula 1d as defined in claim 87: 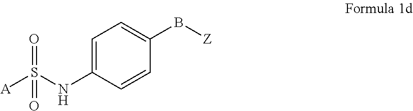
wherein A, B and Z are as defined in claim 68;
comprising the steps of:
step a) treating the compound of formula D: 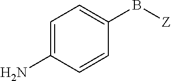
wherein B and Z are as defined in claim 68;
with compound of formula 8 (vi):
A-SO₂—Cl 8 (vi)
wherein A is as defined in claim 68;
in a solvent selected from dichloromethane or chloroform in pyridine as the base at room temperature for 1-2 h; and
step b) hydrolysis of the compound of formula 1d;
wherein Z is: 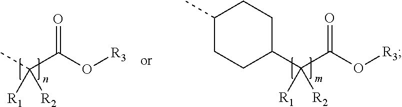
R₁, R₂ and n are as defined in claim 68; and
R₃ is (C₁-C₁₂)-alkyl;
by reaction with aqueous LiOH in a solvent selected from THF or methanol or a mixture thereof, at room temperature for 2-16 h into the corresponding carboxylic acid, the compound of formula 1d (wherein R₃ is H); and conversion of the said carboxylic acid obtained into its corresponding pharmaceutically acceptable salt.

105. A process for the preparation of a compound of formula 1e according to claim 88: 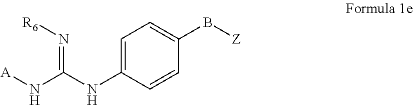
wherein A, B, Z and R₆ are as defined in claim 68;
comprising the steps of:
step a) reacting the compound of formula 1b: 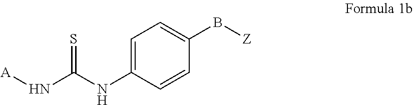
wherein A, B and Z are as defined in claim 68, with the compound of formula 8 (vii):
R₆—NH₂ 8 (vii)
wherein R₆ is as defined in claim 68;
in presence of mercuric oxide (HgO) in methanol as the solvent at room temperature for 1-3 h; and
step b) hydrolysis of the compound of formula 1e;
wherein Z is: 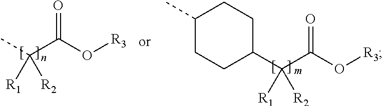
R₁, R₂ and n are as defined in claim 68; and
R₃ is (C₁-C₁₂)-alkyl;
by reaction with aqueous LiOH in a solvent selected from THF or methanol or a mixture thereof, at room temperature for 2-16 h into the corresponding carboxylic acid, the compound of formula 1e (R₃ is H); and conversion of the said carboxylic acid obtained into its corresponding pharmaceutically acceptable salt.

106. A process for the preparation of a compound of formula D as defined in claim 100, represented by the following formula 8: 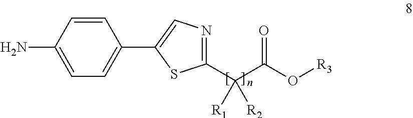
wherein R₃ is (C₁-C₁₂)-alkyl; R₁, R₂ and n are as defined in claim 68;
comprising the steps of:
step a) preparation of compound of formula 3: 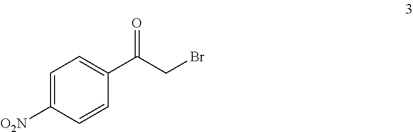 [SMILES: O=C(CBr)c1ccc([N+](=O)[O-])cc1]
by bromination of compound of formula 2: 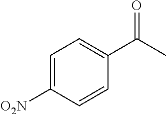
in presence of anhydrous aluminium chloride (AlCl₃) as the catalyst in dry ether at a temperature range of 0° C. to 35° C. for 4-8 h;
step b) reacting the compound of formula 3 with hexamethylene tetramine in a solvent selected from dichloromethane or chloroform at room temperature for 4-16 h, to yield the corresponding hexamine salt, which may be hydrolysed using hydrochloric acid (HCl) in a solvent selected from ethanol or methanol to yield compound of formula 4; 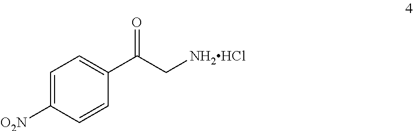
step c) preparing a compound of formula 5: 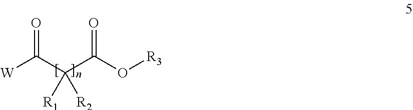
wherein W is OH; R₃ is (C₁-C₁₂)-alkyl; R₁, R₂ and n are as defined in claim 68;
by the partial hydrolysis of the corresponding diester by using methanolic potassium hydroxide (KOH) or by treatment of the corresponding anhydride with concentrated sulfuric acid (H₂SO₄) in methanol;
step d) reacting compound of formula 5 with isobutylchloroformate in presence of N-methylmorpholine as the base in a solvent selected from THF or N,N-dimethylformamide (DMF) at a temperature range of −20° C. to −30° C. to form a carbonate, which is further reacted with the compound of formula 4 in presence of triethylamine as the base in a solvent selected from THF or DMF at room temperature, to yield compound of formula 6; 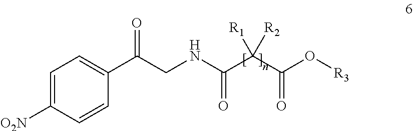
step e) refluxing the compound of formula 6 with Lawesson's reagent in a solvent selected from 1,4-dioxane or THF, at a temperature range of 60° C. to 110° C., to yield the compound of formula 7; and 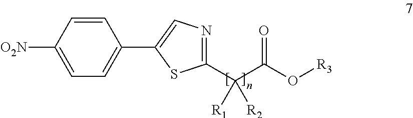
step f) reducing the compound of formula 7 with iron (Fe) and ammonium chloride (NH₄Cl) as the reducing agent in a solvent mixture of ethanol (EtOH), THF and water at a temperature range of 70° C. to 80° C. for 2-6 h to yield compound of formula 8.

107. A process for the preparation of a compound of formula D as defined in claim 100 represented by the following formula 18: 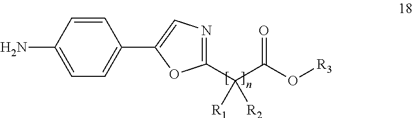
wherein R₃ is (C₁-C₁₂)-alkyl; R₁, R₂ and n are as defined in claim 68;
comprising the steps of:
step a) preparation of compound of formula 17: 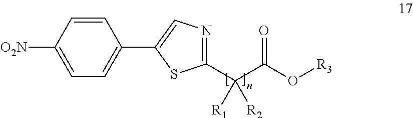
by refluxing compound of formula 6: 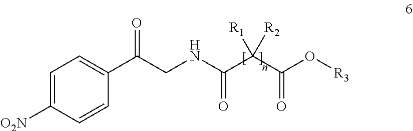
with phosphoryl chloride (POCl₃), optionally in presence of acetonitrile as the solvent, at a temperature range of 80° C. to 110° C. for 2-3 h; and
step b) reducing the compound of formula 17 with Fe and NH₄Cl as the reducing agent in a solvent mixture of EtOH, THF and water at a temperature range of 70° C. to 80° C. for 2-6 h to yield compound of formula 18.

108. A process for the preparation of a compound of formula D as defined in claim 100 represented by following formula 29: 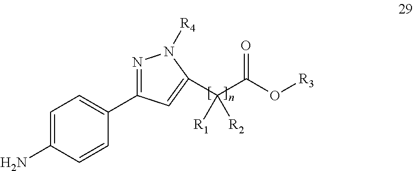
wherein R₃ is (C₁-C₁₂)-alkyl; R₁, R₂, R₄ and n are as defined in claim 68;
comprising the steps of:
step a) preparation of a compound of formula 27: 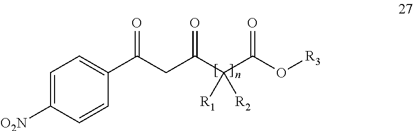
by reacting a compound of formula 2: 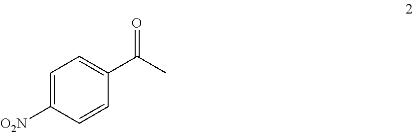
with a compound of formula 5: 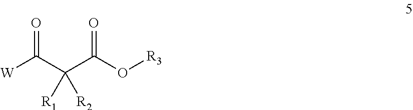
wherein W is OH; R₃ is (C₁-C₁₂)-alkyl; R₁, R₂ and n are as defined in claim 68;
in a solvent selected from toluene, ethanol or THF at a temperature range of 60° C. to 120° C., optionally in the presence of a base selected from sodium hydride, potassium carbonate or cesium carbonate;
step b) refluxing compound of formula 27 with a compound of formula 27 (i); 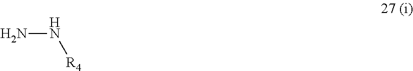
wherein R₄ is as defined in claim 68; in a solvent selected from ethanol or methanol at a temperature of 60° C. to 85° C. to yield the compound of formula 28; and 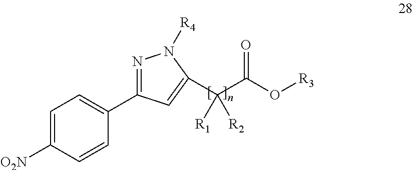
step c) reducing compound of formula 28 with Fe and NH₄Cl as the reducing agent in a solvent mixture of EtOH, THF and water at a temperature range of 70° C. to 80° C. for 2-6 h to yield compound of formula 29.

109. A process for the preparation of a compound of formula D as defined in claim 100 represented by the following formula 43: 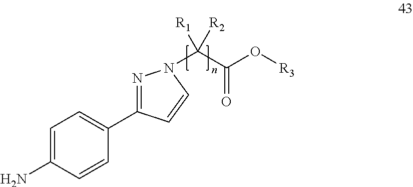
wherein R₃ is (C₁-C₁₂)-alkyl; R₁, R₂ and n are as defined in claim 68;
comprising the steps of:
step a) treating a compound of formula 39: 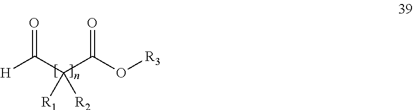
wherein R₃ is (C₁-C₁₂)-alkyl; R₁, R₂ and n are as defined in claim 68;
with tert-butyl carbazate followed by reaction with sodium triacetoxy borohydride or borane-THF complex at a temperature range of 0° C. to 35° C. for 7 h, to yield the compound of formula 40; 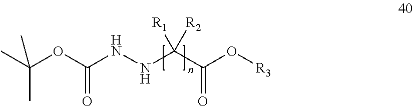
step b) reacting the compound of formula 40 with 4N HCl in dioxane at a temperature range of 25° C. to 50° C. for 10 h, to yield the compound of formula 41; 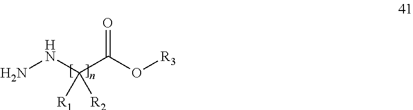
step c) reacting compound of formula 38: 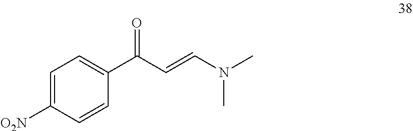 [SMILES: CN(C)/C=C/C(=O)c1ccc([N+](=O)[O-])cc1]
with the compound of formula 41 in a solvent selected from EtOH or methanol at a temperature range of 50-80° C. to yield the compound of formula 42; and 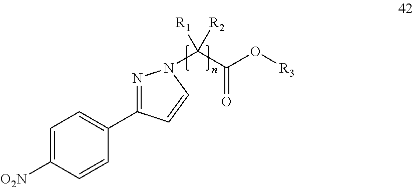
step d) reducing the compound of formula 42 with Fe and NH₄Cl as the reducing agent in a solvent mixture of EtOH, THF and water at a temperature range of 70° C. to 80° C. for 2-6 h to yield compound of formula 43.

110. A process for the preparation of a compound of formula D as defined in claim 100 represented by the following formula 56: 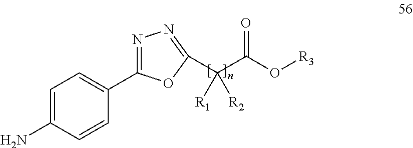
wherein R₃ is (C₁-C₁₂)-alkyl; R₁, R₂ and n are as defined in claim 68;
comprising the steps of:
step a) reacting a compound of formula 53: 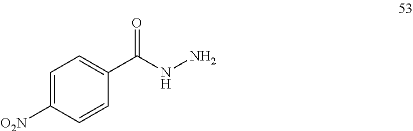 [SMILES: NNC(=O)c1ccc([N+](=O)[O-])cc1]
with a compound of formula 5: 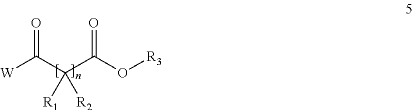
wherein W is OH; R₃ is (C₁-C₁₂)-alkyl; R₁, R₂ and n are as defined in claim 68;
in dichoromethane as the solvent in presence of triethylamine as the base at room temperature for 10 to 18 h, to yield the compound of formula 54; 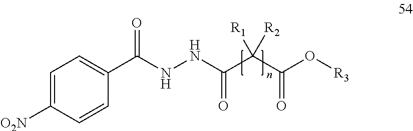
step b) refluxing compound of formula 54 with POCl₃, optionally in the presence acetonitrile as the solvent, at a temperature range of 80° C. to 110° C. for 2-3 h, to obtain the compound of formula 55; and 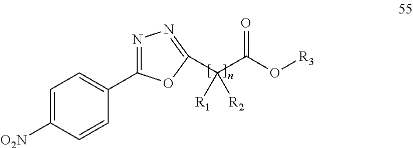
step c) reducing compound of formula 55 with Fe and NH₄Cl as the reducing agent in a solvent mixture of EtOH, THF and water at a temperature range of 70° C. to 80° C. for 2-6 h to yield compound of formula 56.

111. A process for the preparation of a compound of formula D as defined in claim 100 represented by the following formula 66: 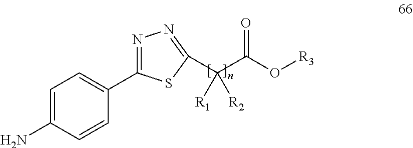
wherein R₃ is (C₁-C₁₂)-alkyl; R₁, R₂ and n are as defined in claim 68;
comprising the steps of:
step a) refluxing the compound of formula 54: 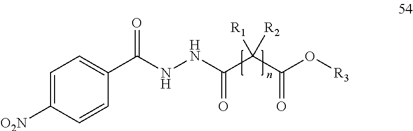
with Lawesson's reagent in a solvent selected from 1,4-dioxane or THF, at a temperature range of 80° C. to 110° C., to yield the compound of formula 65; and 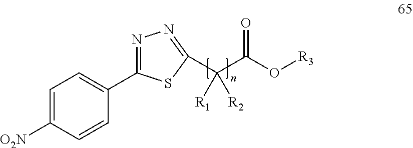
step b) reducing compound of formula 65 with Fe and NH₄Cl as the reducing agent in a solvent mixture of EtOH, THF and water at a temperature range of 70° C. to 80° C. for 2-6 h to yield compound of formula 66.

112. A process for the preparation of a compound of formula D as defined in claim 100 represented by the following formula 78: 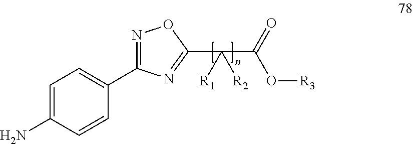
wherein R₃ is (C₁-C₁₂)-alkyl; R₁, R₂ and n are as defined in claim 68;
comprising the steps of:
step a) reacting compound of formula 75: 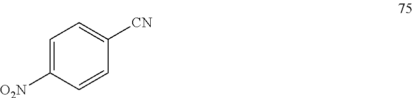 [SMILES: N#Cc1ccc([N+](=O)[O-])cc1]
with hydroxylamine hydrochloride in presence of potassium carbonate (K₂CO₃) as the base in a solvent selected from methanol (MeOH) or ethanol (EtOH) at a temperature range of 50° C. to 80° C. for 4-10 h, to yield the compound of formula 76; 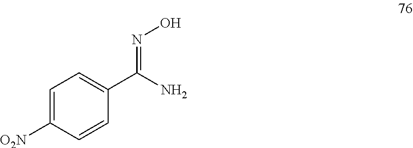 [SMILES: N/C(=N\O)c1ccc([N+](=O)[O-])cc1]
step b) reacting compound of formula 76 with compound of formula 5: 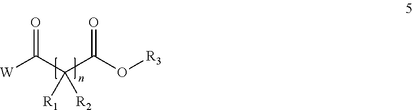
wherein W is OH; R₃ is (C₁-C₁₂)-alkyl; R₁, R₂ and n are as defined in claim 68;
in a solvent selected from dichloromethane or chloroform in presence of carbonylimidazole as the coupling reagent at room temperature for 8-10 h; followed by cyclisation by refluxing in toluene at a temperature range of 100° C. to 130° C. for 18 h, to yield the compound of formula 77; and 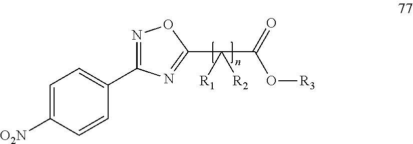
step c) reducing compound of formula 77 with Fe and NH₄Cl as the reducing agent in a solvent mixture of EtOH, THF and water at a temperature range of 70° C. to 80° C. for 2-6 h to yield compound of formula 78.

113. A process for the preparation of a compound of formula D as defined in claim 100 represented by the following formula 90: 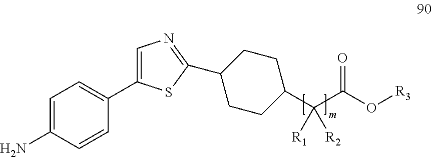
wherein R₃ is (C₁-C₁₂)-alkyl; R₁, R₂ and m are as defined in claim 68;
comprising the steps of:
step a) preparation of compound of formula 87: 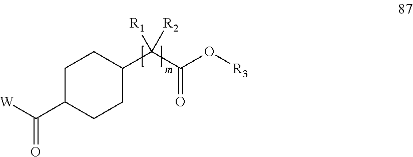
wherein W is OH; R₃ is (C₁-C₁₂)-alkyl; R₁, R₂ and m are as defined in claim 68;
comprising the steps of:
(i) reacting compound of formula A: 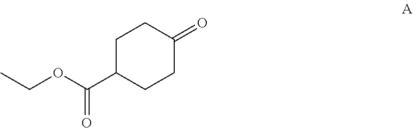
with tert-butyl-2-(diethoxy phosphoryl)acetate in presence of sodium hydride as the base in THF as the solvent at 0° C. for 1 h, followed by at room temperature for 16 h, to yield the compound of formula B; 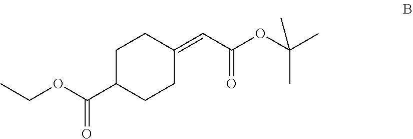 [SMILES: CCOC(=O)C1CCC(=CC(=O)OC(C)(C)C)CC1]
(ii) hydrogenation of compound of formula B in presence of palladium over carbon (Pd/C) as the catalyst in a solvent selected from ethyl acetate, ethanol or methanol at room temperature, to yield the compound of formula C; and 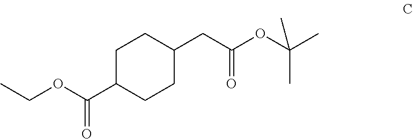 [SMILES: CCOC(=O)C1CCC(CC(=O)OC(C)(C)C)CC1]
(iii) partial hydrolysis of the compound of formula C in the presence of potassium hydroxide (KOH) as the base in a solvent mixture of methanol and water at room temperature for 2 h to yield the compound of formula 87 wherein m=1;
step b) reaction of compound of formula 4: 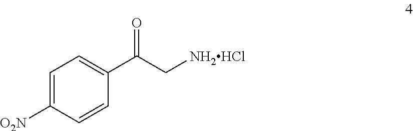
with compound of formula 87 in presence of benzotriazol-1-yloxy)tris(dimethylamino) phosphonium hexafluorophosphate (BOP) as the coupling agent and triethylamine as the base in a solvent selected from DMF or THF at a temperature range of 50° C. to 60° C. to yield the compound of formula 88; 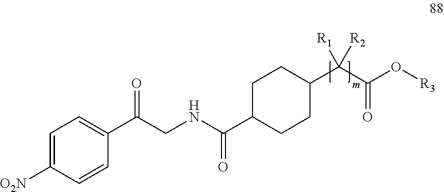
step c) refluxing compound of formula 88 with Lawesson's reagent in a solvent selected from 1,4-dioxane or THF, at a temperature range of 80° C. to 110° C., to yield the compound of formula 89; and 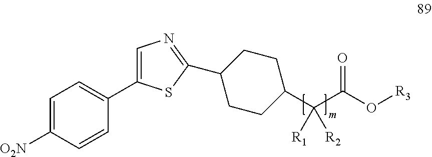
step d) reducing the compound of formula 89 with Fe and NH₄Cl as the reducing agent in a solvent mixture of EtOH, THF and water at a temperature range of 70° C. to 80° C. for 2-6 h to yield compound of formula 90.

114. A process for the preparation of a compound of formula D as defined in claim 100 represented by the following formula 100: 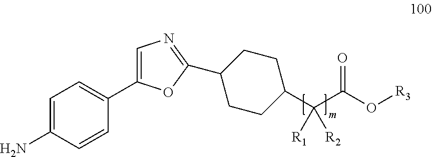
wherein R₃ is (C₁-C₁₂)-alkyl; R₁, R₂ and m are as defined in claim 68;
comprising the steps of:
step a) refluxing compound of formula 88; 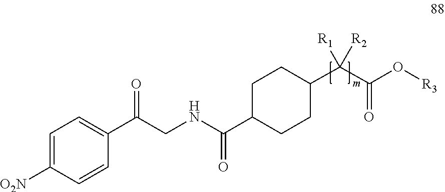
with POCl₃, optionally in presence of acetonitrile as the solvent, at a temperature range of 80° C. to 110° C. for 2-3 h, to yield the compound of formula 99; and 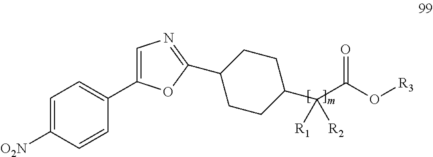
step b) reducing the compound of formula 99 with Fe and NH₄Cl as the reducing agent in a solvent mixture of EtOH, THF and water at a temperature range of 70° C. to 80° C. for 2-6 h to yield compound of formula 100.

115. A process for the preparation of a compound of formula D as defined in claim 100 represented by the following formula 110: 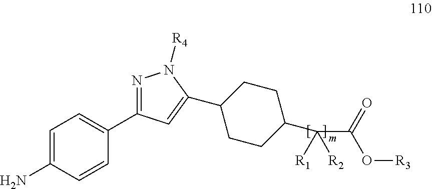
wherein R₃ is (C₁-C₁₂)-alkyl; R₁, R₂, R₄ and m are as defined in claim 68;
comprising the steps of:
step a) reacting compound of formula 2: 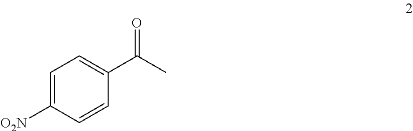
with compound of formula 87: 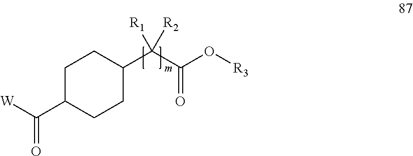
wherein W is OH; R₃ is (C₁-C₁₂)-alkyl; R₁, R₂ and m are as defined in claim 68;
in a solvent selected from toluene, ethanol or THF at a temperature range of 60° C. to 120° C., optionally in presence of a base selected from sodium hydride, potassium carbonate or cesium carbonate, to yield the compound of formula 87(1); 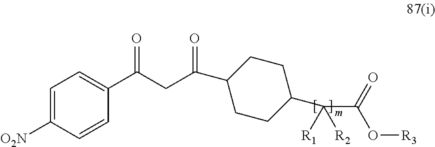
which may be refluxed with compound of formula 27 (i); 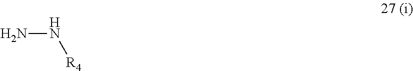
wherein R₄ is as defined in claim 68; in a solvent selected from ethanol or methanol at a temperature of 60° C. to 85° C., to yield the compound of formula 109; and 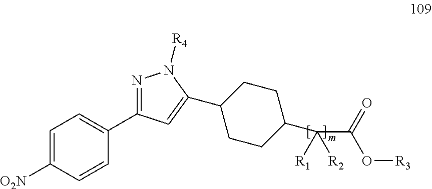
step b) reducing the compound of formula 109 with Fe and NH₄Cl as the reducing agent in a solvent mixture of EtOH, THF and water at a temperature range of 70° C. to 80° C. for 2-6 h to yield compound of formula 110.

116. A process for the preparation of a compound of formula D as defined in claim 100 represented by the following formula 123: 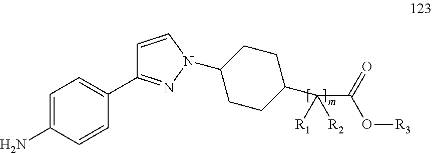
wherein R₃ is (C₁-C₁₂)-alkyl; R₁, R₂ and m are as defined in claim 68;
comprising the steps of:
step a) reacting compound of formula 119: 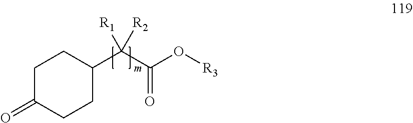
wherein R₃ is (C₁-C₁₂)-alkyl; R₁, R₂ and m are as defined in claim 68;
with tert-butyl carbazate followed by reaction with sodium triacetoxy borohydride or borane-THF complex at a temperature range of 0° C. to 35° C. for 7 h, to yield the compound of formula 120; 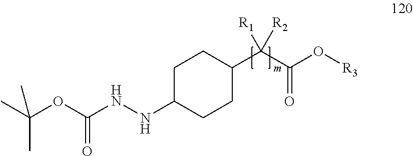
step b) reacting compound of formula 120 with 4N HCl in dioxane at a temperature range of 25° C. to 50° C. for 10 h, to yield the compound of formula 121; 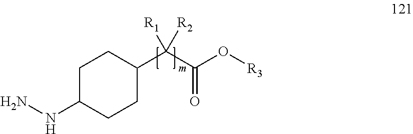
step c) reacting compound of formula 38: 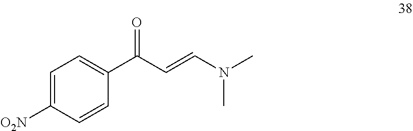 [SMILES: CN(C)/C=C/C(=O)c1ccc([N+](=O)[O-])cc1]
with the compound of formula 121 in a solvent selected from EtOH or methanol at a temperature range of 50° C. to 80° C., to yield the compound of formula 122; and 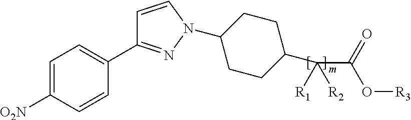
step d) reducing compound of formula 122 with Fe and NH₄Cl as the reducing agent in a solvent mixture of EtOH, THF and water at a temperature range of 70° C. to 80° C. for 2-6 h to yield compound of formula 123.

117. A process for the preparation of a compound of formula D as defined in claim 100 represented by the following formula 134: 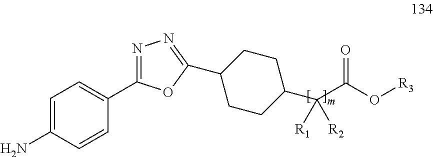
wherein R₃ is (C₁-C₁₂)-alkyl; R₁, R₂ and m are as defined in claim 68;
comprising the steps of:
step a) reaction of compound of formula 53: 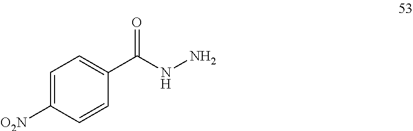 [SMILES: NNC(=O)c1ccc([N+](=O)[O-])cc1]
with compound of formula 87: 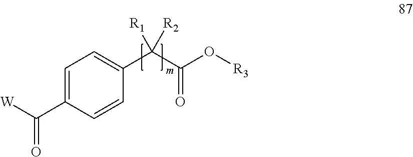
wherein W is OH; R₃ is (C₁-C₁₂)-alkyl; R₁, R₂ and m are as defined in claim 68;
in dichloromethane as the solvent in the presence of triethylamine as the base at room temperature for 10-18 h, to yield the compound of formula 132; 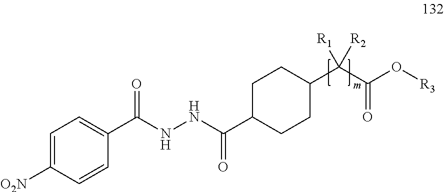
step b) refluxing compound of formula 132 with POCl₃, optionally in the presence of acetonitrile as the solvent, at a temperature range of 80° C. to 110° C. for 2-3 h, to obtain the compound of formula 133; and 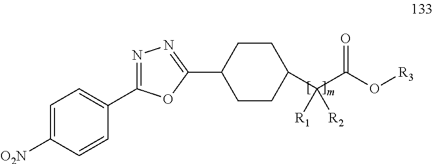
step c) reducing compound of formula 133 with Fe and NH₄Cl as the reducing agent in a solvent mixture of EtOH, THF and water at a temperature range of 70° C. to 80° C. for 2-6 h to yield compound of formula 134.

118. A process for the preparation of a compound of formula D as defined in claim 100 represented by the following formula 145: 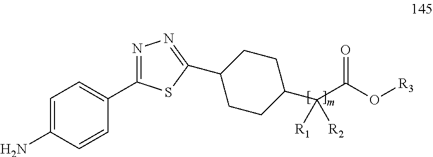
wherein R₃ is (C₁-C₁₂)-alkyl; R₁, R₂ and m are as defined in claim 68;
comprising the steps of:
step a) reacting compound of formula 53: 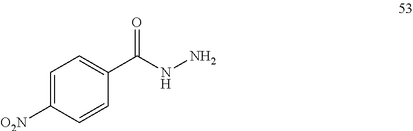 [SMILES: NNC(=O)c1ccc([N+](=O)[O-])cc1]
with compound of formula 87; 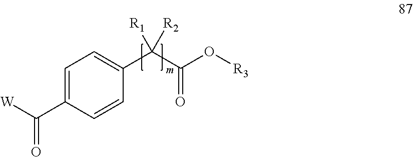
wherein W is OH; R₃ is (C₁-C₁₂)-alkyl; R₁, R₂ and m are as defined in claim 68;
in dichloromethane as the solvent in presence of triethylamine as the base at room temperature for 10-18 h, to yield the compound of formula 143; 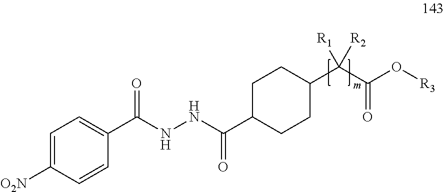
step b) refluxing compound of formula 143 with Lawesson's reagent in a solvent selected from 1,4-dioxane or THF, at a temperature range of 80° C. to 110° C., to yield the compound of formula 144; and 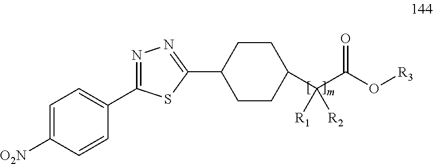
step c) reducing compound of formula 144 with Fe and NH₄Cl as the reducing agent in a solvent mixture of EtOH, THF and water at a temperature range of 70° C. to 80° C. for 2-6 h to yield compound of formula 145.

119. A process for the preparation of a compound of formula D as defined in claim 100 represented by the following formula 155: 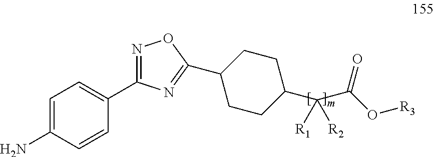
wherein R₃ is (C₁-C₁₂)-alkyl; R₁, R₂ and m are as defined in claim 68;
comprising the steps of:
step a) reacting compound of formula 76: 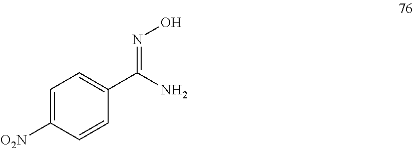 [SMILES: N/C(=N\O)c1ccc([N+](=O)[O-])cc1]
with compound of formula 87: 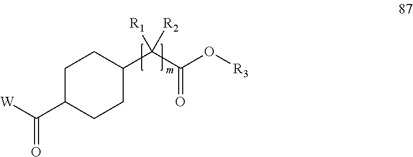
wherein W is OH; R₃ is (C₁-C₁₂)-alkyl; R₁, R₂ and m are as defined in claim 68;
in a solvent selected from dichloromethane or chloroform in presence of carbonylimidazole as the coupling agent at room temperature for 8-10 h, followed by cyclisation by refluxing in toluene at a temperature range of 100° C. to 130° C. for 18 h, to yield the compound of formula 154; and 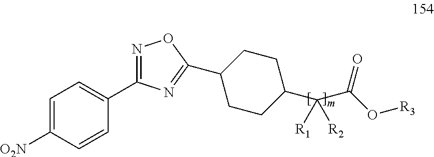
step b) reducing compound of formula 154 with Fe and NH₄Cl as the reducing agent in a solvent mixture of EtOH, THF and water at a temperature range of 70° C. to 80° C. for 2-6 h to yield compound of formula 155.

120. A process for the preparation of a compound of formula D as defined in claim 100 represented by the following formula 166: 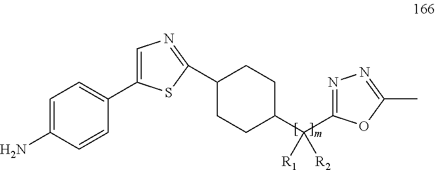
wherein R₁, R₂ and m are as defined in claim 68;
comprising the steps of:
step a) reacting compound of formula 89: 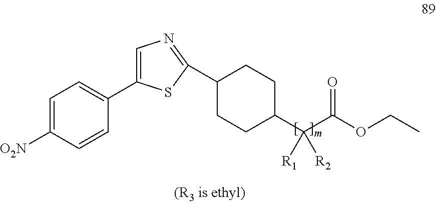
with hydrazine hydrate in ethanol as the solvent at 80° C. for 4-6 h to yield the compound of formula 164; 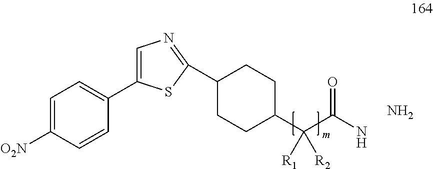
step b) reacting compound of formula 164 with acetic acid and POCl₃ at 80° C. for 2-4 h to yield the compound of formula 165; and 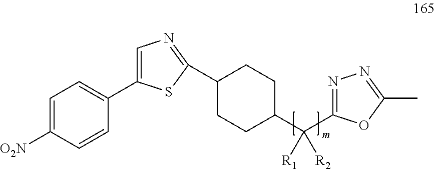
step c) reducing compound of formula 165 with Fe and NH₄Cl as the reducing agent in a solvent mixture of EtOH, THF and water at a temperature range of 70° C. to 80° C. for 2-6 h to yield compound of formula 166.

121. A process for the preparation of a compound of formula D as defined in claim 100 represented by the following formula 171: 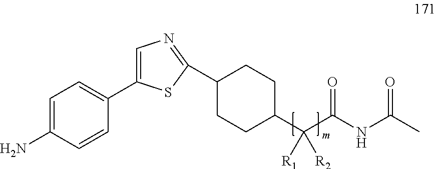
wherein R₁, R₂ and m are as defined in claim 68;
comprising the steps of:
step a) hydrolyzing compound of formula 89 (R₃=ethyl): 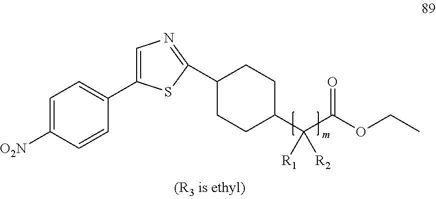
by reacting with sodium hydroxide (NaOH) in a solvent mixture of THF and methanol at room temperature for 16 h to yield compound of formula 89 (R₃═H): 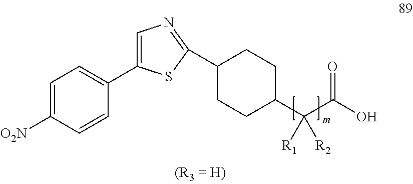
step b) reacting the compound of formula 89 (R₃═H) with oxalyl chloride and N-hydroxyacetamidine in a solvent selected from dichloroethane (DCE) or dioxane at room temperature for 32 h to yield compound of formula 169; 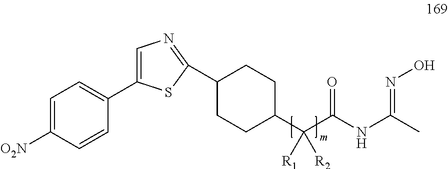
step c) heating the compound of formula 169 in DMF in a microwave at 120° C. for 2-4 h to yield compound of formula 170; and 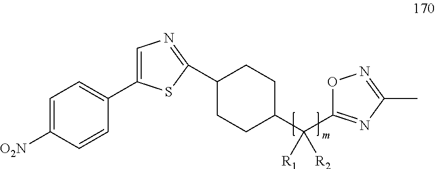
step d) reducing compound of formula 170 with Fe and NH₄Cl as the reducing agent in a solvent mixture of EtOH, THF and water at a temperature range of 70° C. to 80° C. for 2-6 h to yield compound of formula 171.

122. A process for the preparation of a compound of formula D as defined in claim 100 represented by the following formula 172: 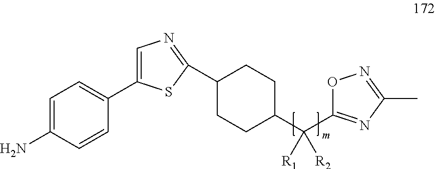
wherein R₁, R₂ and in are as defined in claim 68;
by reducing the compound of formula 170: 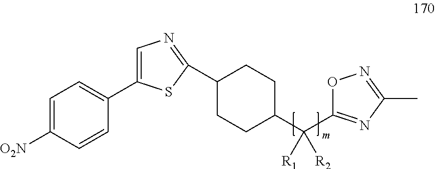
with sodium sulphide as the reducing agent in a solvent mixture of dioxane and water at a temperature range of 70° C. to 90° C. for 1 h.

123. A process for the preparation of a compound of formula D as defined in claim 100 represented by the following formula 179: 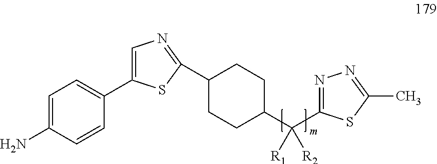
wherein R₁, R₂ and m are as defined in claim 68;
comprising the steps of:
step a) reacting compound of formula 89: 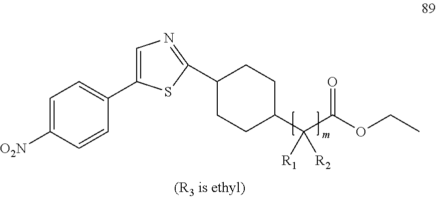
with oxalyl chloride and acetic hydrazide in a solvent selected from DCE or dioxane at room temperature for 32 h to yield compound of formula 177; 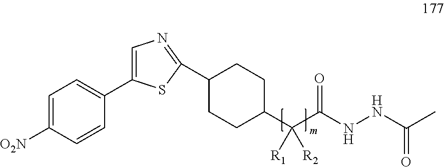
step b) reacting the compound of formula 177 with Lawesson's reagent in a solvent selected from 1,4-dioxane or xylene at a temperature range of 100° C. to 150° C., to yield compound of formula 178; and 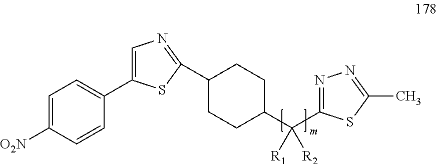
step c) reducing the compound of formula 178 with Fe and NH₄Cl as the reducing agent in a solvent mixture of EtOH, THF and water at a temperature range of 70° C. to 80° C. for 2-6 h to yield compound of formula 179.

124. A process for the preparation of a compound of formula D as defined in claim 100 represented by the following formula 192: 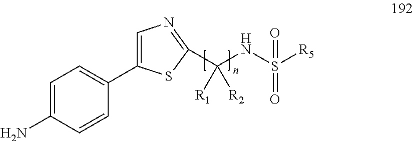
wherein R₁, R₂, R₅ and n are as defined in claim 68;
comprising the steps of:
step a) reacting compound of formula 186: 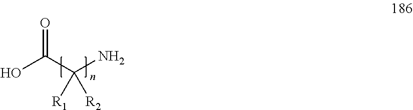
wherein R₁, R₂ and 11 are as defined in claim 68;
with ter-Butyloxycarbonyl anhydride (BOC-anhydride) in presence of sodium bicarbonate (NaHCO₃) as the base in a solvent mixture of acetonitrile and water at a temperature range of 0° C. to room temperature for 16 h to yield compound of formula 187; 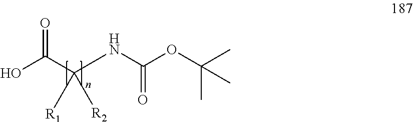
step b) reacting the compound of formula 187 with 2-amino-1-(4-nitrophenyl)ethanone hydrochloride in presence of a mixture of HATU and triethylamine as the base in DMF as the solvent at room temperature for 3-5 h to yield compound of formula 188; 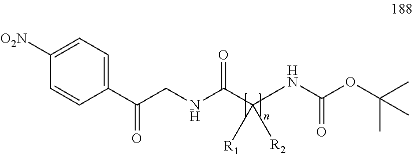
step c) reacting the compound of formula 188 with Lawesson's reagent by refluxing in a solvent selected from 1,4-dioxane or THF, at a temperature range of 60° C. to 110° C. for 1-3 h, to yield the compound of formula 189; 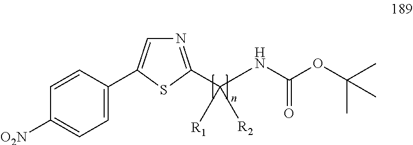
step d) reacting the compound of formula 189 with HCl in 1,4-dioxane at room temperature for 20 h to yield the compound of formula 190; 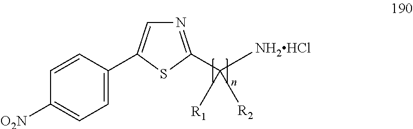
step e) reacting the compound of formula 190 with the reagent:
R₅SO₂Cl or (R₅SO₂)₂O,
wherein R₅ is as defined in claim 68;
in presence of triethylamine as the base in dichloromethane at room temperature for 1-3 h to yield compound of formula 191; and 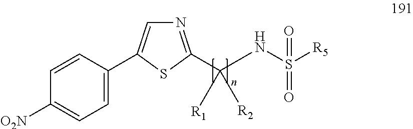
step 1) reducing the compound of formula 191 with Fe and NH₄C1 as the reducing agent in a solvent mixture of EtOH, THF and water at a temperature range of 70° C. to 80° C. for 2-6 h to yield compound of formula 192.

125. A process for the preparation of a compound of formula D as defined in claim 100 represented by the following formula 206: 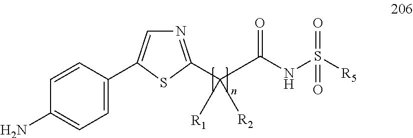
wherein R₁, R₂, R₅ and n are as defined in claim 68;
comprising the steps of:
step a) hydrolyzing compound of formula 7 (R₃ is methyl): 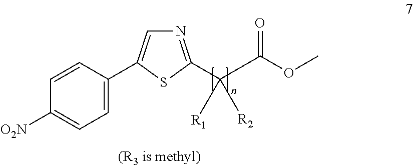
using 1N NaOH in a solvent mixture of THF and methanol at room temperature for 16-24 h to yield compound of formula 7 (R₃ is H); 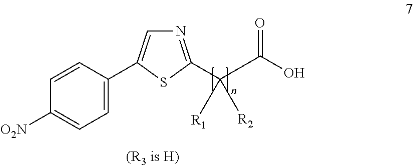
step b) refluxing the compound of formula 7 (R₃ is H) with the reagent:
R₅SO₂NH₂,
wherein R₅ is defined in claim 68;
in presence of isobutyl chloroformate in presence of a base selected from N-Methyl morpholine and 1,8-Diazabicyclo[5.4.0]undec-7-ene (DBU) in THF for 16 h to yield compound of formula 205; 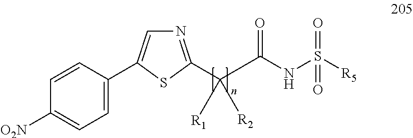
step c) reducing the compound of formula 205 with Fe and NH₄Cl as the reducing agent in a solvent mixture of EtOH, THF and water at a temperature range of 70° C. to 80° C. for 2-6 h to yield compound of formula 206.

126. A process for the preparation of a compound of formula D as defined in claim 100 represented by the following formula 215: 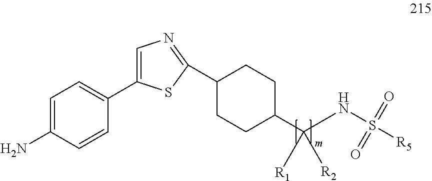
wherein R₁, R₂, R₅ and m are as defined in claim 68;
comprising the steps of:
step a) reacting a compound of formula 210: 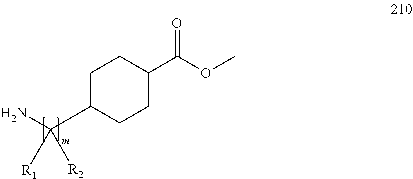
wherein R₁, R₂ and n are as defined in claim 68;
is reacted with triflic anhydride in presence of N,N-diisopropylethylamine (DIPEA) as the base in dichloromethane as the solvent at room temperature for 16 h to yield compound of formula 211; 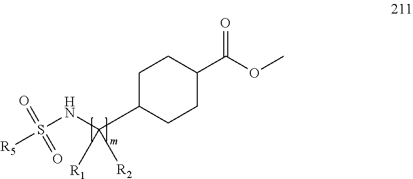
step b) hydrolyzing the compound of formula 211 using LiOH in as THF at room temperature for 16 h to yield the compound of formula 212; 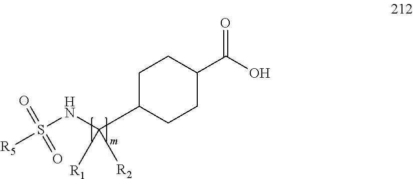
step c) reacting the compound of formula 212 with 2-amino-(4-nitro)acetophenone hydrochloride in the presence of 2-(7-Aza-1H-benzotriazole-1-yl)-1,1,3,3-tetramethyluronium hexafluorophosphate (HATU) as the coupling agent and triethyl amine as the base in DMF as the solvent at room temperature for 3-5 h to yield the compound of formula 213; 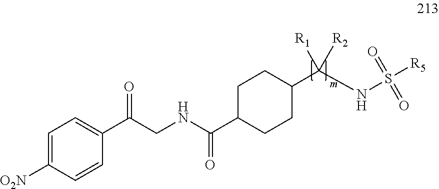
step d) refluxing the compound of formula 213 with Lawesson's reagent in a solvent selected from 1,4-dioxane or THF, at a temperature range of 60° C. to 110° C., to yield the compound of formula 214; and 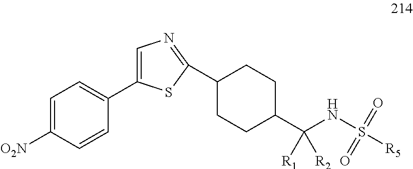
step c) reducing compound of formula 214 with Fe and NH₄Cl as the reducing agent in a solvent mixture of EtOH, THF and water at a temperature range of 70° C. to 80° C. for 2-6 h to yield compound of formula 215.

127. A process for the preparation of a compound of formula D as defined in claim 100 represented by the following formula 223: 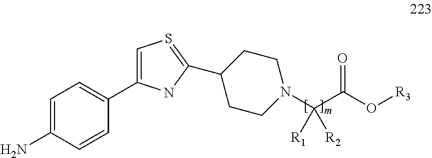
wherein R₁, R₂ and m are as defined in claim 68;
comprising the steps of:
step a) refluxing a compound of formula 3: 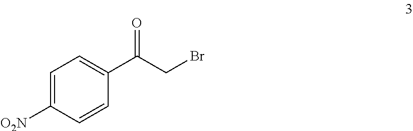 [SMILES: O=C(CBr)c1ccc([N+](=O)[O-])cc1]
with the compound of formula 219: 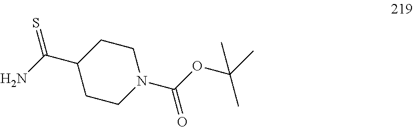 [SMILES: CC(C)(C)OC(=O)N1CCC(C(N)=S)CC1]
at a temperature range of 75° C. to 85° C. for 3-5 h to yield the compound of formula 220; 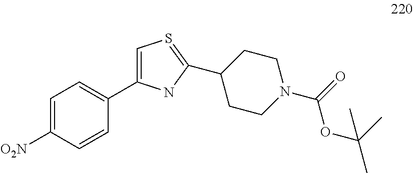
step b) reacting the compound of formula 220 with 1N HCl in ethyl acetate as the solvent at room temperature to yield the compound of formula 221; 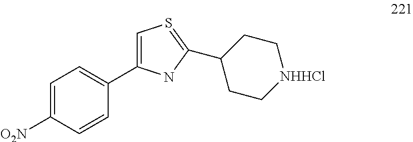
step c) reacting the compound of formula 221 with the reagent: 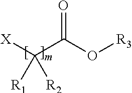
wherein X is halogen; m, R₁, R₂ and R₃ are as defined in claim 68;
in presence of a base such as triethylamine in toluene at a temperature range of 100° C. to 120° C. to yield the compound of formula 222; and 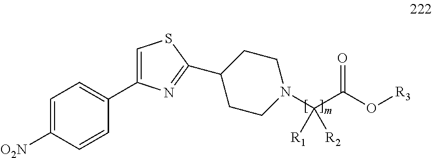
step d) reducing the compound of formula 222 with Fe and NH₄Cl as the reducing agent in a solvent mixture of EtOH, THF and water at a temperature range of 70° C. to 80° C. for 2-6 h, to yield compound of formula 223.

128. A process for the preparation of a compound of formula D as defined in claim 100 represented by the following formula 229: 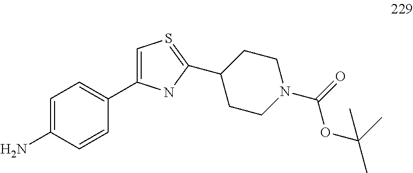
comprising the steps of:
step a) reacting compound of formula 4: 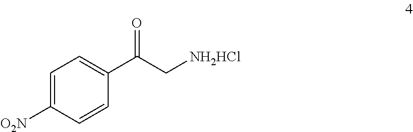
with a compound of formula 226: 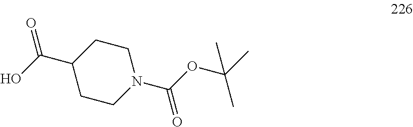 [SMILES: CC(C)(C)OC(=O)N1CCC(C(=O)O)CC1]
in presence of DIPEA as the base in DMF as the solvent in presence of HATU as the coupling agent at room temperature for 30 min to 1 h to yield the compound of formula 227; 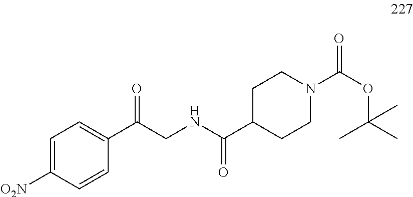 [SMILES: CC(C)(C)OC(=O)N1CCC(C(=O)NCC(=O)c2ccc([N+](=O)[O-])cc2)CC1]
step b) reacting the compound of formula 227 is reacted with Lawesson's reagent in dioxane at 50° C. to 70° C. for 2-4 h to yield the compound of formula 228; and 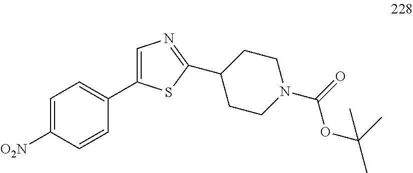 [SMILES: CC(C)(C)OC(=O)N1CCC(c2ncc(-c3ccc([N+](=O)[O-])cc3)s2)CC1]
step c) reducing the compound of formula 228 with Fe and NH₄Cl as the reducing agent in a solvent mixture of EtOH, THF and water at a temperature range of 70° C. to 80° C. for 2-6 h, to yield compound of formula 229.

129. A process for the preparation of a compound of formula D as defined in claim 100 represented by the following formula 234: 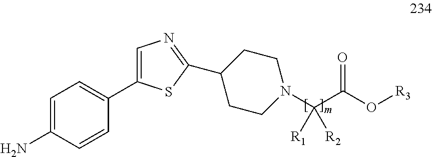
wherein R₁, R₂, R₃ and m are as defined in claim 68;
comprising the steps of:
step a) reacting compound of formula 228: 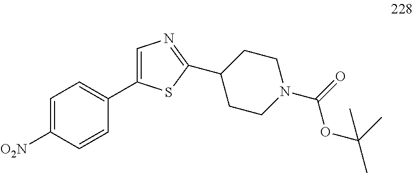 [SMILES: CC(C)(C)OC(=O)N1CCC(c2ncc(-c3ccc([N+](=O)[O-])cc3)s2)CC1]
with 1N HCl in ethyl acetate as the solvent at room temperature to yield the compound of formula 232; 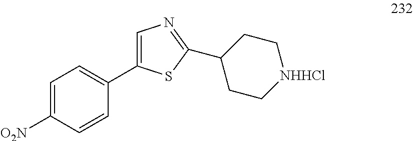
step b) reacting the compound of formula 232 is reacted with the reagent: 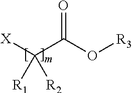
wherein X is halogen; m, R₁, R₂ and R₃ are as defined in claim 68;
in presence of triethylamine as the base in toluene at a temperature range of 100° C. to 120° C. to yield the compound of formula 233; and 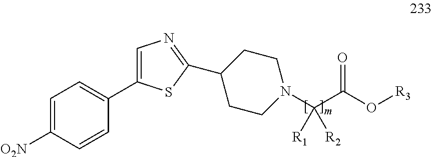
step c) reducing the compound of formula 233 with Fe and NH₄Cl as the reducing agent in a solvent mixture of EtOH, THF and water at a temperature range of 70° C. to 80° C. for 2-6 h, to yield compound of formula 234.

130. A process for the preparation of a compound of formula D as defined in claim 100 represented by compound of formula 240: 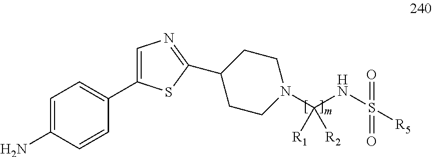
wherein R₁, R₂, R₅ and m are as defined in claim 68;
comprising the steps of:
step a) reacting compound of formula 232: 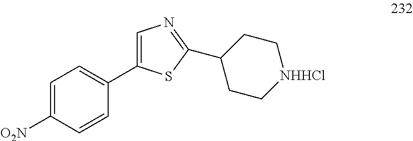
with t-butyl 2-bromoethylcarbamate in the presence of potassium carbonate (K₂CO₃) as the base in DMF as the solvent at a temperature range of 50° C. to 80° C. for 2-4 h to yield the compound of formula 237; 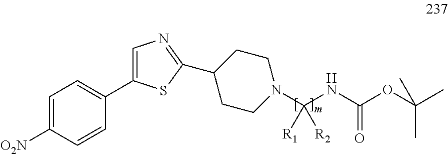
step b) reacting the compound of formula 237 with HCl in a solvent selected from isopropanol or methanol at room temperature for 12-15 h to yield the compound of formula 238; 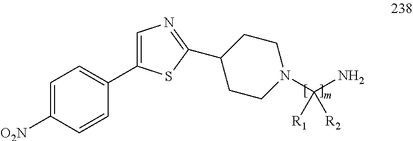
step c) reacting the compound of formula 238 with trifle anhydride in dichloromethane as the solvent and triethylamine as the base at room temperature for 10-16 h to yield the compound of formula 239; and 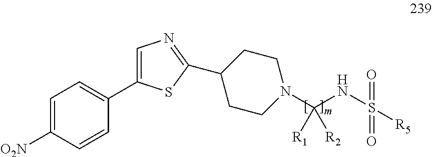
step d) reducing the compound of formula 239 with Fe and NH₄Cl as the reducing agent in a solvent mixture of EtOH, THF and water at a temperature range of 70° C. to 80° C. for 2-6 h, to yield compound of formula 240.

131. A process for the preparation of a compound of formula D as defined in claim 100 represented by the following formula 243: 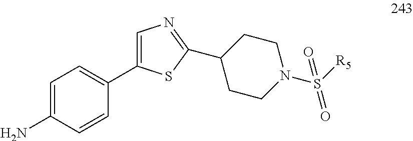
wherein R₅ is as defined in claim 68;
comprising the steps of:
step a) reacting compound of formula 232: 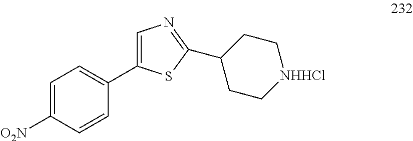
with a reagent:
R₅SO₂Cl or R₅(SO₂)₂O;
wherein R₅ is as defined in claim 68; in the presence of triethylamine as the base in dichloromethane as the solvent at room temperature for 16 h to yield the compound of formula 242; and 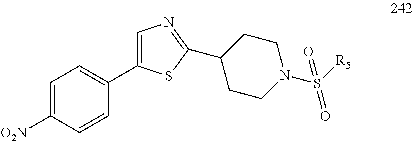
step b) reducing the compound of formula 242 with Fe and NH₄Cl as the reducing agent in a solvent mixture of EtOH, THF and water at a temperature range of 70° C. to 80° C. for 2-6 h. to yield compound of formula 243.

132. A process for the preparation of a compound of formula D as defined in claim 100 represented by the following formula 249: 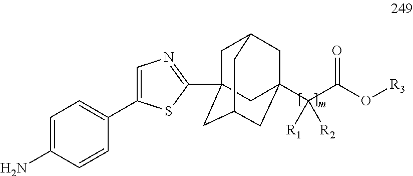
wherein R₁, R₂, R₃ and m are as defined in claim 68;
comprising the steps of:
step a) reacting the compound of formula 245: 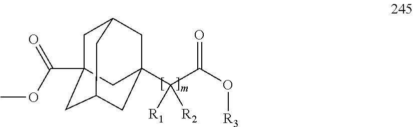
with KOH as the base in methanol at a temperature range of 60° C. to 80° C. for 16 h followed by acidification with dilute HCl to yield the compound of formula 246; 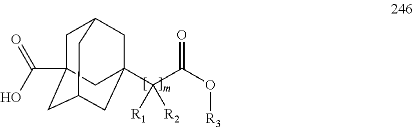
step b) reacting the compound of formula 246 with the compound of formula 4 in presence of HATU as the coupling agent and DIPEA as the base in DMF at room temperature for 30 min to 2 h to yield the compound of formula 247; 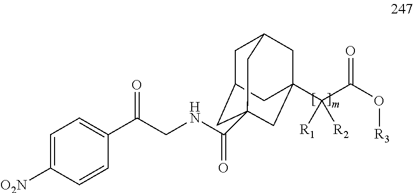
step c) reacting the compound of formula 247 with Lawesson's reagent dioxane as the solvent at 50° C. to 70° C. for 2-4 h to yield the compound of formula 248; and 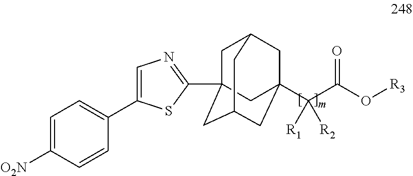
step d) reducing the compound of formula 248 with Fe and NH₄Cl as the reducing agent in a solvent mixture of EtOH, THF and water at a temperature range of 70° C. to 80° C. for 2-6 h, to yield compound of formula 249.
